(19) United States
(12) Patent Application Publication
(10) Pub. No.: US 2012/0178771 A1
(43) Pub. Date: Jul. 12, 2012

(54) Oral Pharmaceutical Compositions of Buprenorphine and Method of Use

(76) Inventors: Najib BABUL, Blue Bell, PA (US); Ashish Kumar Rehni, Paitala (IN)

(21) Appl. No.: 13/229,505

(22) Filed: Sep. 9, 2011

Related U.S. Application Data
(63) Continuation of application No. PCT/US2009/005394, filed on Sep. 28, 2009.
Application No. PCT/US2009/005394 is a continuation-in-part of application No. PCT/US2009/001502, filed on Mar. 9, 2009.
(60) Provisional application No. 61/064,505, filed on Mar. 8, 2008.

(51) Int. Cl. A61K 31/439

(52) U.S. Cl. 514/282

(57) ABSTRACT

The present invention is directed to oral, therapeutically effective pharmaceutical compositions of buprenorphine and it pharmaceutically acceptable salts and the use thereof, including delayed onset and controlled release dosage forms. The present invention is also directed delayed onset, rapid release dosage forms and delayed onset, extended release dosage forms of oral buprenorphine which provide robust efficacy and reduced potential for abuse and misuse.

This application is a Continuation-in-Part and claims the benefit of PCT Patent Application No. PCT/US2009/001502, filed on Mar. 9, 2009, which has a priority date of Mar. 8, 2008 and claims the benefit of U.S. Provisional patent Application No. 61/064,505, the contents of each being incorporated herein by reference it their entirety.

### FIELD OF THE INVENTION

The present invention is directed to oral, therapeutically effective pharmaceutical compositions of buprenorphine and it pharmaceutically acceptable salts and the use thereof, including delayed onset and controlled release dosage forms. The present invention is also directed delayed onset, rapid release dosage forms and delayed onset, extended release dosage forms of oral buprenorphine which provide robust efficacy and reduced potential for abuse and misuse.

### BACKGROUND TO THE INVENTION

While opioid analgesics are administered by a wide variety of routes and methods, including oral, oro-transmucosal, buccal, sublingual, via NG-tubes, rectal, intranasal, inhalational, topical, transdermal, intravenous, subcutaneous, intramuscular, epidural and intrathecal, the oral route is by far the most preferred route for most patients and medical settings.

The strong preference and universal acceptance of the oral route include simplicity of drug administration, patient convenience, lower drug costs, and reduced complexity of manufacturing, suitability for repeated and chronic administration and, in many cases, reliable therapeutic effect.

In some cases, drugs need to be administered by a non-oral route. Among the reasons for the use of non-oral routes are lack of therapeutic effect, lack of a consistent, reliable or robust effects, the need for rapid onset of effect, contraindications to oral drug administration, reduced safety by the oral route and the lack of availability of the oral route (for example due to GI obstruction, nausea, vomiting, GI obstruction, obtundation or coma).

Over the past several decades, there have been extensive research, development and commercialization efforts to advance alternatives to orally ingested drug administration, including oro-transmucosal, buccal, sublingual, intranasal, inhalational, topical, transdermal, intravenous, subcutaneous, intramuscular, epidural, intrathecal and transdermal routes. Although there have been remarkable advances in alternatives to orally ingested drugs, the oral route continues to be the preferred route of drug administration.

Unfortunately, when given orally, many drugs produce suboptimal clinical effects, including a slow onset, short duration, inconsistent or variable response, reduced therapeutic benefit, inefficient clinical response and reduced safety

In many cases, the prediction of absent or significantly poor therapeutic outcomes by the oral route has led to the abandonment of its evaluation through oral ingestion. Such drugs are frequently being used only by a non-oral route (e.g., parenteral route, intranasal or transdermal).

Currently, medical practitioners may choose from several well-accepted classes of pharmaceutical agents in their attempts to alleviate and prevent pain. Nonlimiting examples of agents used include nonsteroidal anti-inflammatory agents (NSAIDs), e.g., aspirin, ibuprofen, ketoprofen, diclofenac; opioids, e.g., morphine, hydromorphone, hydrocodone, oxycodone, tramadol, and codeine; cyclooxygenase-2 (COX-2) selective NSAIDs, e.g., celecoxib, valdecoxib, etoricoxib, lumiracoxib, and rofecoxib; acetaminophen; tricyclic antidepressants, e.g., amitriptyline, desipramine, nortriptyline; non-tricyclic antidepressants, e.g., doxepin, duloxetine, paroxetine, venlafaxine; antiepileptics, e.g., gabapentin, pregabalin, carbamazepine, oxcarbazepine, lamotrigine; voltage sensitive N-type calcium channel blockers, e.g., ziconotide and alpha adrenergic agonists, e.g., clonidine.

Of the many challenges that occur when pharmacologically treating any disease or pathological condition, including pain, alleviating the symptoms without causing counterproductive side effects is often the greatest. This challenge presents itself when medical practitioners use medicinal agents to treat pain. Although the aforementioned pharmacological classes are frequently effective for the treatment of certain types of pain, the chronic and acute use of these analgesic agents produces a number of significant, undesirable side effects.

Morphine is extensively utilized for the management of severe pain due to its global availability, extensive clinical experience, significant pharmacokinetic and pharmacodynamic data, low cost and the availability of various extended release formulations (Babul et. al, J Clin Pharmacol, 1998; 38:74-81). However, the incidence and severity of side effects limits the use of morphine in some patients (Hagen and Babul, Cancer 1997; 79:1428-1437). In patients with renal impairment, morphine's principal metabolites, morphine-3-glucuronide and morphine-6-glucuronide can accumulate. Morphine-3-glucuronide accumulation has been implicated in hyperalgesia, respiratory stimulation, and behavioral excitatory properties through nonopioid receptor mechanisms. Morphine-6-glucuronide accumulation has been implicated in increasing levels of nausea and sedation in patients with renal impairment (Babul and Darke, Clin Pharm Ther, 1993; 54:286-92). Similarly, the principal metabolite of hydromorphone, hydromorphone-3-glucuronide can accumulate in patients with renal impairment and has been found to be more neurotoxic than morphine-3-glucuronide (Babul and Darke, Pain, 1992; 51:260-61; Hagen et al., J Clin Pharmacol, 1995; 35:38-45; Babul et al., J Pain Symptom Manage, 1995; 10:184-86; Wright et al., Life Sci, 1998; 63:401-11; Wright et al., Life Sci, 2001; 69:409-20.).

An important goal of analgesic therapy in chronic pain is to achieve continuous relief of pain. Regular administration of an analgesic is generally required to ensure that the next dose is given before the effects of the previous dose have worn off (Principles of Analgesic Use in the Treatment of Acute Pain and Cancer Pain, Fifth Ed., American, Pain Society (2003); Evidence Based Report of the U.S. Agency for Healthcare Research and Quality (AHRQ) on the Management of Cancer Pain, Report No. 35, AHRQ Publication No. 02-E002, October 2001; Carr et al. J Nat Cancer Inst Monograph 2004; 32:23-31; Agency for Health Care Policy and Research Clinical Practice Guidelines for Cancer Pain Management, Guideline No. 9, AHCPR Publication No. 94-0592, March 1994; Agency for Health Care Policy and Research Clinical Practice Guideline for Acute Pain Management, Guideline No. 1, AHCPR Publication No. 92-0032, February, 1992; Guideline for the Management of Cancer Pain in Adults, American Pain Society, 2005; Guideline for the Management of Pain in Osteoarthritis, Rheumatoid Arthritis, and Juvenile Chronic Arthritis, 2^{nd} Ed., American Pain Society, 2002). Over the past decade, the paradigm of regular administration of an analgesic (sometimes referred to as around the clock, scheduled or time contingent analgesia) has expanded beyond chronic pain to also cover many acute pain states where the pain lasts longer than a few hours or a few days (e.g., pain after surgery, trauma, etc).

Conventional (so called “immediate-release”, “rapid release” or “short acting”) opioid analgesics have been demonstrated to provide short-lived plasma levels, thereby requiring dosing every 4-6 hours in chronic pain. In contrast, extended release oral opioids are designed to maintain effective plasma levels throughout a 12 or 24-hour dosing interval. Use of extended release opioids can result in fewer interruptions in sleep, reduced dependence on caregivers, improved compliance, enhanced quality of life outcomes, and increased control over the management of their pain. In addition, such formulations can provide more constant plasma concentrations and clinical effects, less frequent peak to trough fluctuations and fewer side effects, compared with short acting opioids (Babul et al. Journal of Pain and Symptom Management 2004; 28:59-71; Matsumoto et al., Pain Medicine 2005; 6:357-66; Dhaliwal et al., Journal of Pain Symptom Management 1995; 10:612-23; Hays et al., Cancer 1994; 74:1808-16; Arkinstall et al., Pain 1995; 62:169-78; Hagen et al., Journal of Clinical Pharmacology 1995; 35:38-45; Peloso et al., Journal of Rheumatology 2000; 27:764-71).

The introduction of extended release morphine (MS Contin®) revolutionized the management of cancer pain. MS Contin® gained widespread acceptance due to its global availability, significant pharmacokinetic and pharmacodynamic data, and the convenience of an extended-release formulation. However, the incidence and severity of side effects limits the use of morphine in some patients (Hagen and Babul, Cancer 1997; 79:1428-37).

Clinicians treating cancer pain with opioids have reported significant variability among patients in efficacy and side effects with available opioid analgesics. Patients with poor analgesic efficacy or safety outcomes on one opioid frequently tolerate another opioid well. This clinical observation led to the development of oxycodone ER (OxyContin®). Due to the limitations associated with extended release morphine noted above and the “stigma” associated with its use (i.e., association with addiction, advanced cancer, dying and death), extended release oxycodone gained rapid acceptance by patients with chronic non-cancer pain. However, its widespread use for the treatment of chronic non-malignant pain was also associated with its diversion into the non-medical supply for use both by addicts and recreational drug users

Among the many side effects of opioids are nausea, vomiting, constipation, sedation, fatigue, pruritus, blurred vision, urinary retention, respiratory depression, convulsions, mood changes and alterations of the endocrine and autonomic nervous systems. Many of these side effects are sufficiently bothersome as to require: i) use of additional medications to treat the iatrogenic symptoms; ii) more intensive patient management; iii) use of lower doses that leave patients in continued pain; or iv) in other cases, complete discontinuation of analgesic therapy. Opioids can also produce potentially fatal respiratory depression at high doses. (Evidence Based Report of the U.S. Agency for Healthcare Research and Quality (AHRQ) on the Management of Cancer Pain, Report No. 35, AHRQ Publication No. 02-E002, October 2001; Can et al. J Nat Cancer Inst Monograph 2004; 32:23-31; Agency for Health Care Policy and Research Clinical Practice Guidelines for Cancer Pain Management, Guideline No. 9, AHCPR Publication No. 94-0592, March 1994; Guideline for the Management of Cancer Pain in Adults, American Pain Society, 2005).

The Evidence Based Report of the U.S. Agency for Healthcare Research and Quality (AHRQ) on the Management of Cancer Pain (Report No. 35, AHRQ Publication No. 02-E002, October 2001; Carr et al. J Nat Cancer Inst Monograph 2004; 32:23-31) supports the three tier approach. “The first tier, for mild to moderate pain, consists of NSAIDs and acetaminophen with or without adjuvant medications. As pain escalates or persists, treatment progresses to the second tier, in which a weak opioid, such as codeine or hydrocodone, is added to the NSAID with or without an adjuvant drug.” “If pain still persists, treatment progresses to the third tier: substitution of the “weak” opioid for a “strong” opioid (i.e., one more readily titrated to doses with greater analgesic efficacy). The latter category includes morphine, hydromorphone, methadone, fentanyl, and levorphanol, all full opioid agonists at the morphine or mu receptor.” The AHRQ report further notes that “if pain relief is not achieved at the maximum recommended dose of a particular NSAID or opioid, it should be discontinued and another drug from the same class tried before abandoning that class. Case series indicate that on an individual basis, other drugs from the same class may prove more effective or be better tolerated.”

The Agency for Health Care Policy and Research Clinical Practice Guidelines for Cancer Pain Management (Guideline No. 9, AHCPR Publication No. 94-0592, March 1994) states: “It is usually advisable to observe the patient's response to several different opioids, sequentially, before switching routes of administration or trying an anesthetic, neurosurgical, or other invasive approach to relieve persistent pain. For example, patients who experience dose-limiting sedation, nausea, or mental clouding on oral morphine should be switched to an equianalgesic dose of hydromorphone or fentanyl. The dose of the second opioid should then be adjusted. Sequential analgesic trials should be based on regular assessments of pain, with continuous attention to antineoplastic and noninvasive nonpharmacologic therapies”.

According to the recently issued American Pain Society Guideline for the Management of Cancer Pain in Adults (2005, pp. 59-60), “For patients who experience inadequate pain relief, or an unacceptable level of side effects from a specific opioid that limits dose escalation, pain control can be achieved through opioid rotations. For example, patients who experience dose-limiting sedation, nausea, or mental clouding from oral morphine can be switched to an equianalgesic dose of hydromorphone or fentanyl. Dosage of the second opioid should then be adjusted so that relief is achieved with an acceptable level of side effects. It is usually advisable to observe a patient's response to several different opioids, administered sequentially, before switching routes of administration or trying an anesthetic, neurosurgical, or other invasive approach to relieve persistent cancer pain”.

A number of oral immediate release formulations of opioid analgesics have been described in the art, including codeine, hydrocodone, hydromorphone, isomethadone, levorphanol, meperidine, methadone, morphine, oxycodone, oxymorphone, pentazocine, propoxyphene, tapentadol, tramadol, or their pharmaceutically acceptable salts.

A number of oral extended release formulations of opioid analgesics have been developed or commercialized, including morphine, hydromorphone, oxycodone, hydrocodone and oxymorphone.

Buprenorphine or [5α,7α(S)]-17-(Cyclopropylmethyl)-α-(1,1-dimethylethyl)-4,5-epoxy-18,19-dihydro-3-hydroxy-6-methoxy-α-methyl-6,14-ethenomorphinan-7-methanol or it pharmaceutically acceptable salts have been used for the treatment of a variety of medical conditions, including pain and opioid addiction disorders. It is a semi-synthetic opioid first derived in 1966 from thebaine, an alkaloid from the poppy Papaver somniferum.

Currently, buprenorphine is a Schedule III narcotic. According to the Drug Enforcement Administration (DEA), which enforces the controlled substances laws and regulations of the United States, a Schedule III drug has a low potential for abuse relative to the drugs or other substances in Schedule II, II or I. For comparison purposes, morphine, fentanyl, meperidine, methadone, oxycodone and hydromorphone, the most commonly used opioids are all Schedule II opioids.

There are significant practical and marketing benefits to opioid analgesics with >Schedule II. Prescriptions for Schedule II controlled substances cannot be refilled. A new prescription must be issued. Prescriptions for Schedules III controlled substances may be refilled up to five times in six months. Additionally, prescriptions for Schedule II controlled substances must be written and be signed by the practitioner. Prescriptions for Schedules III may by written, oral or transmitted by fax.

Buprenorphine is a partial agonist at the mu (μ)-opioid receptor. There is greater resistance on the part of physicians and patients to the use of the more potent and full opioid analgesics for non-cancer pain. There is also a greater risk of drug abuse and drug diversion with the full opioid agonists, such as morphine, fentanyl, meperidine, methadone, oxycodone and hydromorphone. Because of the intrinsic activity profile of buprenorphine, it has been classified as a partial opioid agonist at the mu-opioid receptor. Similar to its affinity at the mu receptor, buprenorphine also has a high affinity for the kappa-opioid receptor. However, because of a lack of intrinsic activity at the kappa receptor, it has been classified as a kappa-receptor antagonist. The partial agonist properties of buprenorphine at the mu receptor, together with its high affinity and slow dissociation from the receptor, give rise to its unique pharmacological profile when compared with full mu-opioid agonists such as morphine, including: (i) the ceiling effect in buprenorphine's opioid receptor activity which provides a greater margin of safety (e.g., ceiling effect on respiratory depression) than full agonists such as morphine and methadone; (ii) the potential to block the effects of a full opioid agonist taken after administration of adequate doses of buprenorphine; (iii) the potential to antagonize the effects of a full mu opioid agonist and cause a precipitated withdrawal in opioid-dependent patients according to their level of dependence and the dose and time since the last administration of the full mu agonist; and (iv) the low intrinsic activity at the opioid receptor which provides significantly reduced reinforcing properties when compared to full opioid agonists used for treatment (e.g. morphine or methadone) or illicitly (e.g. heroin).

Thus, buprenorphine may provide a safer alternative to the full opioid agonists, both in terms of opioid effects and in terms of the risk of physical dependence, addiction and drug diversion.

There is a need for new oral pharmaceutical compositions of buprenorphine which are therapeutically effective and also have a reduced potential for abuse.

Although buprenorphine has lower risk of abuse, it does carry a real risk of drug addiction, drug diversion and drug abuse. Without being bound by theory, for an opioid to have appeal as an abusable drug among recreational drug users or addicts, it must produce rapid onset of euphoric or psychic effects. In other words, a temporal relationship between drug ingestion and the appearance of discernible euphoric or psychic effects is essential to its abuse and high “street value” among recreational drug users and addicts. Unfortunately, when given orally to recreational drug users or addicts, many opioid produce measurable euphoric or psychic effects soon after administration, usually within 15 to 120 minutes. The applicant has found a novel way to deter or minimize the abuse of oral buprenorphine among recreational drug users and drug addicts breaking the temporal relationship between drug ingestion and the rapid appearance of discernible euphoric or psychic effects by delaying the release of the buprenorphine.

To the applicant's knowledge, (i) the therapeutic benefit of orally ingested buprenorphine in immediate release, modified release or extended release form has heretofore not been not been tested or established for any medical condition, including pain, addiction disorders and other opioid responsive disorders; (ii) no orally ingested controlled release formulations of buprenorphine have been has heretofore been developed or commercialized; and (iii) no orally ingested modified release, duodenal release, jejunal release, ileal release, ileo-colonic release, or colonic release formulations of buprenorphine have been has heretofore been developed or commercialized.

Bullingham et al (British Journal of Clinical Pharmacology, 1981; 12:863-67) evaluated the efficacy of orally administered acetaminophen (paracetamol) alone, and a combination of paracetamol plus buprenorphine versus placebo. Buprenorphine was not administered alone. Pain was assessed at baseline and again at 0.5, 0.75, 1, 1.5, 2, 3, 4 and 6 hours after study medication. There were no significant differences between the groups in analgesia over the 6 hours.

The applicant has surprisingly discovered that oral buprenorphine administered in modified release form provides robust therapeutic effects.

The applicant has surprisingly discovered that oral buprenorphine provides is more tolerable and provides fewer side effects than sublingual buprenorphine.

The applicant has surprisingly discovered that that oral delivery of delayed onset, ileal delivery, and colonic delivery dosage buprenorphine provides significantly more robust therapeutic effects that oral immediate release buprenorphine dosage forms which are released into the stomach.

Buprenorphine has been commercially available as a parenteral formulation for intravenous and intramuscular use. According to the FDA's Orange Book, parenteral buprenorphine was approved prior to Jan. 1, 1982. Buprenorphine has been widely reported to have very poor oral bioavailability and is believed to be ineffective when given orally. For this reason, pharmaceutical companies have made elaborate efforts to develop alternative non-invasive methods of delivering buprenorphine into systemic circulation. Foremost among these methods is the sublingual delivery of buprenorphine for the treatment of pain (sublingual buprenorphine [Temgesic®] was approved in the U.K. in 1992). This approach has provided only modest efficacy and has been a commercially failure in a number of countries.

On Oct. 8, 2002, two new sublingual formulations of buprenorphine were approved by the FDA in the USA for the “treatment of opioid dependence” but not for pain. One formulation contains buprenorphine alone (Subutex®) and the other contains buprenorphine in combination with naloxone in an optimized ratio of 4 parts buprenorphine base to 1 part naloxone base (Suboxone®). According to the FDA approved U.S. prescribing information, “Suboxone® and Subutex® tablets should be placed under the tongue until they are dissolved. For doses requiring the use of more than two tablets, patients are advised to either place all the tablets at once or alternatively (if they cannot fit in more than two tablets comfortably) place two tablets at a time under the tongue. Either way, the patients should continue to hold the tablets under the tongue until they dissolve; swallowing the tablets reduces the bioavailability of the drug. To ensure consistency in bioavailability, patients should follow the same manner of dosing with continued use of the product.”

According to the manufacturer's patient instructions approved by the FDA on how to take Suboxone® or Subutex®, “(i) Put the tablets under your tongue and let them melt. This will take 2 to 10 minutes. Do not chew or swallow the tablets. The medicine will not work this way and you may get withdrawal symptoms; (ii) if your doctor tells you to take more than 1 tablet, you will be told to: (a) take all tablets at the same time together under your tongue, or take 2 tablets, put them under your tongue. After they melt, put the next tablet or tablets under your tongue right away; (b) hold the tablets under your tongue until they melt completely. The medicine will not work if swallowed and you may get withdrawal symptoms. (c) Do not change the way you are told to take your medicine or you may get too little or too much medicine.”

Major disadvantages with sublingual administration of buprenorphine include but are not limited to: (i) highly variable pharmacokinetics and pharmacodynamics; (ii) variability of patient's ability to adhere to the instructions about oral retention of drug; (iii) the development of a depot of buprenorphine on in the oral tissue; (iv) an unpleasant taste and after-taste; (v) a sensation of “gagging”; (vi) durability of a robust effect over the course of 24 hours; and (vii) increased risk of drug abuse through tampering of the dosage form and subsequent intravenous, intranasal and inhalational use.

The sublingual formulation has also been repeatedly been shown to be unstable and susceptible to degradation. Methods to deal with this degradation have included use of antioxidants (e.g., ascorbic acid, vitamin E, tocopherol, butylated hydroxytoluene, alpha-lipoic acid, sodium metabisulfite, butylated hydroxyanisole, ascorbyl palmitate, hypophosphorous acid, monothioglycerol, potassium metabisulfite, propyl gallate, sodium bisulfate, sodium formaldehyde sulfoxylate, sodium sulfite, sodium thiosulfate, sulfur dioxide, tocopherol, or tocopherols excipient), and use of chelating agents (e.g., edetic acid, edetate calcium disodium, edetate disodium or malic acid). Buprenorphine active pharmaceutical ingredient has also been shown to be subject to oxidative degradation under stress conditions, with degradation producing both known and unknown byproducts. In addition to the cost and complexity of manufacture, addition of antioxidants and chelating agents to the dosage form can adversely influence the stability, potency and use of other excipients, adversely influence safety of the patient through iatrogenic or idiosyncratic reactions, adversely influence the safety of workers handling the excipients, adversely influence the efficiency of co-ingested drugs, and interact with the GI environment in a manner that is adverse to the efficiency of the dosage form (see Rowe, Sheskey and Quinn, Handbook of Pharmaceutical Excipients, 6 edition, Pharmaceutical Press; APhA Publications; 2009).

It is widely recognized that the dose of opioids required to treat pain is highly variable. For example, some patients can be managed with a few mg of morphine while other patients require grams of morphine per day (Babul and Hagen, Proceedings of the American Society for Clinical Pharmacology and Therapeutics, Mar. 24-27, 2002, Atlanta, Ga.). For this reason, patients receiving opioids are titrated to clinical effect by increasing the dose. An increase in dose should provide a dose proportional increase in bioavailability. Unfortunately, sublingual buprenorphine fails to provide dose proportional bioavailability at particularly high doses, thereby limiting its clinical utility. For example, a 16 mg sublingual dose of buprenorphine provides only 70% dose normalized bioavailability of a 4 mg sublingual dose.

Another limitation with the sublingual route is the high peak concentration of buprenorphine. Both peak concentrations of opioids and the rate of rise in concentrations have variously been implicated in the causation of various opioid side effects such as nausea, drowsiness, dizziness and (acute) cognitive impairment. Orally administered buprenorphine can be formulated to provide significantly lower peak concentrations than sublingual buprenorphine. Such an effect is also desirable in opioid dependent individuals to minimize the “rush” or euphoric effect of high peak concentrations.

Oral formulations of buprenorphine, with their differential opioid receptor binding and clinical effects have the potential to be a drug of choice in opioid rotation regimens in patients with suboptimal efficacy and/or safety on other orally available opioids (e.g., oxycodone, morphine, oxymorphone, hydrocodone), thereby providing an effective strategic intervention for such patients (Hagen and Babul, Cancer 1997; 79:1428-37).

Other efforts to provide alternative non-invasive methods of delivering buprenorphine into systemic circulation have included intranasal (see, for example, Linhardt et al, Int J Pharm, 2000; 205 (1-2):159-63) and transdermal administration (see, for example, U.S. Pat. Nos. 7,270,830, 6,344,212, 6,004,969, 5,968,547, 5,240,711, and 5,069,909).

Two transdermal formulations of buprenorphine have been commercialized in some countries, including the U.K., for the treatment of pain. One formulation is designed for application to the skin about twice a week (Transtec®) and another formulation is designed for administration once-a-week (BuTrans®). Major disadvantages with transdermal administration of buprenorphine include but are not limited to: (i) cost of goods; (ii) wide inter- and intra-individual variability if rate and extent of absorption; (iii) delay with onset of clinically meaningful therapeutic effect; (iv) poor skin adhesion over sweaty and hairy skin and in tropical climatic zones; (v) cutaneous adverse reactions to buprenorphine and/or its adhesives and other excipients; (vi) reduce flexibility in dose titration in relation to changing clinical status; (vii) the potential for variable absorption in the presence of pronounced fever; (viii) formation of a skin depot of buprenorphine, despite removal of the patch; and (ix) poor adhesion during vigorous physical activity, bathing or water sports.

Other important disadvantages with transdermal administration of buprenorphine include but are not limited to: (i) a delayed and highly variable time to minimum effective plasma concentrations; (ii) a delayed and highly variable time to maximum plasma concentrations; (iii) a delayed time to steady state concentration; (iv) a wide peak to trough plasma concentration fluctuation; (v) a wide variability in peak concentration (Cₘₐₓ); (vi) a wide variability in extent of absorption (AUC_{0-τ}); and (vii) inconsistent delivery over the dosing interval.

Clinical studies of transdermal buprenorphine have shown local skin reactions in approximately one-third of patients. It is believed that a majority of these reactions are due to the patch material or adhesive, since such reactions have been seen in both active and placebo groups in clinical trials. Pruritus and erythema are the most common local reactions and they are usually of mile to moderate intensity.

The development of reliable transdermal formulations is challenging as such formulations must provide consistent delivery of drug over a prolonged period of time when applied under variable conditions (e.g., skin sites of varying adiposities, differing degrees of sweating, changes in temperature with physical activity and fever, and presence of moisture when bathing). Additionally, such formulations need to provide reliable skin adhesion or adhesivity while being painless to remove at the end of the dosing interval.

At the completion of treatment with a transdermal dosage form of buprenorphine, a considerable amount of residual buprenorphine remains in the dosage form. This can readily be extracted and diverted into the illicit drug market.

Addicts and recreational drug also abuse of transdermal opioids through tampering and extraction of drug for subsequent oral ingestion, snorting, inhalation or intravenous injection. Such tampering has been known to include extraction of the active substance from the transdermal reservoir or matrix by needle aspiration, oral ingestion of the active substance from the transdermal system, and solvent extraction of the active substance from the transdermal system.

Transdermal buprenorphine dosage forms are not yet available in the USA; therefore it is difficult to fully predict their likely patterns of abuse. In contrast, transdermal forms of the opioid fentanyl have been widely available in the USA for a considerable period of time. Transdermal fentanyl has been an important addition to the therapeutic armamentarium for the treatment of pain. However, it continues to be abused and misused for its addiction potential (Liapas et al., J Psychopharmacol, 2004; Tharp et al., Am J Forensic Med Pathol, 2004; Jost et al., Dtsch Med Wochenschr, 2004; Milligan et al., J Pain, 2001; Kuhlman et al., J Anal Toxicol, 2003; Arvanitis and Satonik, et al., Am J Emerg Med, 2002; Marquardt et al., Ann Pharmacother, 1995; Poklis, J Toxicol Clin Toxicol, 1995; De Sio et al., Anesthesiology, 1993; Lilleng et al., J Forensic Sci, 2004; Kuhlman et al, J Anal Toxicol, 2003; Reeves and Ginifer, Med J Aust, 2003; Pizon and Brooks, Vet Hum Toxicol, 2004; Nevin, Emerg Med Serv, 2004; Barrueto, Vet Hum Toxicol, 2004; Marquardt and Tharratt, Toxicol Clin Toxicol, 1994).

The abuse of fentanyl patch has involved a variety of methods including steeping the patch in hot water (“fentanyl tea bag”); inhalation of patch contents; solvent extraction, followed by intravenous use; needle aspiration, followed by intravenous use; mechanical extraction, followed by intravenous use; solvent extraction, followed by oral, mL; nasal and inhalation use; mechanical extraction, followed by oral, mL; nasal and inhalation use; transdermal application of the contents of the tampered patch; combustion of the patch, followed by inhalation; and a combination of the above methods.

Underscoring its concerns about drug abuse, recently, the U.S. Food and Drug Administration made a precedential decision rejecting the approval of a bioequivalent (“A/B generic”) patch of transdermal fentanyl, solely of the basis of the amount of fentanyl in the patch, rather than the amount of fentanyl absorbed into systemic circulation compared to the innovator (Duragesic®).

On Jul. 8, 2005, the FDA posted a Safety Alert and on Jul. 15, 2005, they issued a Public Health Advisory about the deaths resulting from use of the transdermal fentanyl patch. At that time, there were 120 deaths reported and the FDA was investigating the deaths to determine if they were due to inappropriate use or defective patches. According to the FDA, the deaths spanned the time period beginning in 1990 when the drug was introduced and stemmed from legitimate use rather than abuse of the medication. FDA noted that it was “investigating reports of death and other serious adverse events related to narcotic overdose in patients using the fentanyl transdermal patch for pain control.” FDA further noted that it “recently conducted a review of fatalities reported to the voluntary adverse event reporting system that were possibly due to unintentional overdose from the fentanyl transdermal patch. In many cases, establishing whether the overdose was unintentional was difficult, because the information provided in the report was incomplete and patients who were being treated with the fentanyl patch often had underlying diseases or conditions that could have contributed to their deaths (such as cancer). Factors identified as possibly related to unintentional overdose included: use of high doses of the fentanyl patch and/or multiple patches (sometimes in combination with other drugs), possible medication errors, accidental exposure (e.g., coming in contact with a discarded patch), application of a heat source to the patch possibly resulting in increased fentanyl absorption, injection or ingestion of the patch contents, and suspected transdermal patch malfunction (e.g., leaking patches). In addition, several patients reported poor adhesion of the patches to the skin.”

In 2005, the Los Angeles Times reported that the Los Angeles County Coroner's office has investigated more than 230 deaths involving fentanyl (FDA Slow to Sound Alarm on Pain Drug Fentanyl's overdose rate and side effects show holes in monitoring medicine on the market, Nov. 25, 2005, Page A1). Of those 230-plus cases, 127 were classified as “accidental deaths,” suggesting that the victims had inadvertently received too much of the drug through the pain patch. According to a report in the LA Times, 115 additional fentanyl deaths were reported from the state of Florida in the year 2004, as well as an alarming rise in emergency-room cases involving the drug. According to the Federal Substance Abuse and Mental Health Services Administration (SAMHSA), emergency-room admissions involving fentanyl rose from 28 in 1998 to 1506 in 2004.

According to an FDA report introduced at a judicial proceeding, between April 2003 and June 2004, a total of 2.5 million Duragesic® patches were recalled due to customer reports of leakage. There have been numerous additional recalls of transdermal fentanyl patches by multiple manufacturers.

Thus there is a need for optimized alternative opioid formulations for the treatment of acute and chronic pain. There is also a need for optimized opioid formulations for the treatment of cancer pain and neuropathic pain. There is also a need for alternative buprenorphine formulations for the treatment of acute pain, chronic pain, cough, dyspnea, addiction disorders and other buprenorphine responsive medical conditions.

There is a need for optimized formulations of non-transdermal opioids that provide reduced pharmacokinetic and pharmacodynamic variability, a rapid onset of effect, a sustained duration of effect, consistent effects over time, improved safety and tolerability. There is also a need for optimized pharmaceutical formulations of oral opioids that deliver the drug into systemic circulation more efficiently and with improved patient and prescriber acceptance.

There is a need for oral (i.e., orally ingested, as opposed to lingual, sublingual or buccal) formulations of buprenorphine that are therapeutically effective for the treatment of various medical conditions, including but not limited to pain and opioid addiction disorders.

The dosage form of the invention also provides pharmaceutical compositions and methods to improve treatment compliance and to deter episodic, occasional, or intermittent use in subjects requiring chronic buprenorphine therapy around the clock or on a time contingent basis by rendering the dosage form therapeutically ineffective or suboptimally effective when taken episodically, intermittently, or occasionally.

The dosage form of the invention also provides pharmaceutical compositions and methods to deter the abuse and misuse of the dosage form by recreational drug users of opioids and opioid addicts by rendering the dosage form devoid of or substantially devoid of euphoria, pleasurable, drug liking or other mood alerting effects when taken on an as needed basis.

The oral route of administration (i.e., oral ingestion) is the most widely used and most widely preferred method of drug administration. It is simple, reliable and readily accessible. Under most conditions of use, particularly outside the hospital setting, it is the recommended method of drug administration. Even in settings of skilled nursing care, where there are technical and human resources to initiate and manage parenteral therapy, the goal is to rapidly transition patients from parenteral medications to oral medications. Some generally cited exceptions to the use of the oral route include: (i) drugs with poor oral bioavailability; (ii) drugs requiring a rapid onset of effect; (iii) where venous access already exists (e.g., in the peri-operative or intensive care setting); (iii) where the oral route provides unreliable or inconsistent clinical effects. Therapeutic administration of buprenorphine alone in immediate release form, sustained release, modified release, delayed onset, duodenal release, jejunal release, ileal release, ileo-colonic release, and colonic release dosage forms has either not been practiced or has been dismissed as unreliable or clinically unacceptable for some of the reasons noted above.

Contrary to this view, the applicant asserts that orally administered buprenorphine can provide acceptable pharmacokinetics, pharmacodynamics, clinical efficacy and safety. Administration of buprenorphine by the oral route provides significantly greater flexibility in dosage form design, clinical utility and patient acceptability. In the USA, buprenorphine is classified as a Schedule III drug. According to the DEA and WHO, Schedule III drugs have lower potential for abuse than the drugs in Schedules I and II (e.g., fentanyl, codeine, hydrocodone, hydromorphone, methadone, meperidine, morphine, oxycodone, and oxymorphone). In addition, since sustained release drugs are the standard of care for the management of many chronic conditions and sustained release opioids are the standard of care for the management of chronic pain, an orally effective buprenorphine, with its reduced abuse potential compared with Schedule II opioids, has the potential to provide fewer interruptions in sleep, reduced dependence on caregivers, improved compliance, enhanced quality of life outcomes, and increased control over the management of their pain. In addition, such formulations can provide more constant plasma concentrations and clinical effects, less frequent peak to trough fluctuations and fewer side effects, compared with sublingual buprenorphine. Similarly, for the treatment of opioid addiction disorders (where sublingual buprenorphine is approved), oral sustained release buprenorphine can provide similar attributes as seen in patients with pain and has the potential to become the standard of care. When compared with sublingual administration, oral immediate and sustained release buprenorphine may be associated with reduced peak to trough fluctuation in concentrations and clinical effects, such as drug craving. Furthermore, in many cases, such dosage forms have a reduced potential for abuse and diversion than sublingual formulations of buprenorphine which are designed to rapidly dissolve in the oral cavity, thereby reducing subsequent intravenous, intranasal and inhalational abuse.

There is a need for oral formulations of buprenorphine that are therapeutically efficient and that can provide: (i) immediate release of the buprenorphine; (ii) modified release of the buprenorphine; (iii) sustained release of the buprenorphine; and (iv) delayed onset, duodenal delivery, jejunal delivery, ileal delivery, ileo-colonic delivery or colonic delivery.

For an oral dosage form, the stability of the active drug in the acidic media of the stomach is important. Furthermore, impurities produced through degradation of the drug substance can have adverse safety consequences. Since the safety (e.g. toxicology, mutagenicity and carcinogenicity) of almost all impurities are not evaluated during drug development, regulatory authorities put strict limits on individual and total impurities, on the assumption that lesser (and fewer) the impurities, the lower the potential risk to patients. Buprenorphine has been found to degrade upon stress degradation in acid and H₂O₂ (oxidation), each producing impurities which are known to the applicant (oxidative stress) and impurities which are unknown to the applicant (oxidative stress and acid stressed). Additionally, the applicant has found that the behavior of oral buprenorphine dosage forms incorporating certain widely used controlled release material shows unexpected deterioration in the dissolution rate of buprenorphine under acidic conditions (increase in release at pH 1.2), providing a rationale in some embodiments of the invention for protection of the dosage form from the acid media of the stomach and release from the dosage form at some point distal to the stomach.

There is a need for pharmaceutical compositions of oral buprenorphine which are stable for a prolonged period and under a variety of environmental conditions. There is also a need for pharmaceutical compositions of oral buprenorphine which do not require anti-oxidants or chelating agents to stabilize the buprenorphine.

The applicant has demonstrated for the first time that oral administration of buprenorphine can produce robust, dose dependent therapeutic effects.

The applicant has demonstrated for the first time that oral administration of buprenorphine in a dosage form that provides extended release of buprenorphine provides robust therapeutic effects.

The applicant has demonstrated for the first time that oral administration of buprenorphine in a dosage form that provides for delayed onset, ileal delivery, ileo-colonic delivery or colonic delivery of buprenorphine provides robust therapeutic effects.

The applicant has demonstrated for the first time that oral administration of buprenorphine that provides for delayed onset and ileal delivery, ileo-colonic delivery or colonic delivery of buprenorphine provides significantly more robust therapeutic effects that following oral immediate release buprenorphine dosage forms which is deposited unsequestered into the stomach.

The applicant has demonstrated that oral dosage forms of the invention can provide robust stability without the need for anti-oxidants or chelating agents to stabilize the buprenorphine.

There is therefore a need for new oral pharmaceutical compositions of buprenorphine and methods for the treatment of pain, opioid dependence, addiction disorders, cough, dyspnea and other buprenorphine responsive medical conditions through targeted gastrointestinal delivery, availability, release, disintegration, dissolution, metabolism and/or absorption

There is therefore a need for new oral pharmaceutical compositions of buprenorphine which are controlled release, modified release, delayed onset, duodenal release, jejunal release, ileal release, ileo-colonic release or colonic release, which provide improved pharmacokinetics, dose proportional absorption, clinical efficacy, safety, tolerability, and reproducibility of clinical response, and methods for the treatment of pain, opioid dependence, addiction disorders, cough, dyspnea and other buprenorphine responsive medical conditions.

There is therefore a need for new oral pharmaceutical compositions of buprenorphine which are controlled release, modified release, delayed onset, duodenal release, jejunal release, ileal release, ileo-colonic release or colonic release, which achieve their objectives at least in part through delivery of the drug into the lower segments of the gastrointestinal tract through time-controlled, pH-controlled, pressure-controlled, enzyme-controlled and hydration controlled.

The safety of sublingual buprenorphine has primarily been evaluated in opioid tolerant and opioid dependent subjects. In such subjects, the incidence of opioid related side effects is known to be much lower than in subjects who are opioid naïve or who are not opioid tolerant. Despite this, sublingual buprenorphine can produce side effects in opioid tolerant and opioid dependent patients, including nausea, vomiting, constipation, and cognitive impairment. In non-addicts receiving opioids under the care of physicians highly experienced in pain management in the controlled setting of an analgesic clinical trial (where treatment dropout rates should be low), approximately 20 to 40% of subjects discontinue treatment within a few days to a few weeks of initiation due to side effects. Therefore, opioid side effects are a substantial deterrent to initiating opioid therapy and they add to patient suffering and the cost of therapy (e.g., drugs to treat the side effects, additional physician visits, etc). There is a need for therapeutically effective formulations of buprenorphine which have lower side effects.

Surprisingly, oral administration of buprenorphine is more tolerable and produces fewer and less intense common opioid side effects compared with sublingual buprenorphine.

Buprenorphine has a long elimination half-life, particularly when measured using optimized venous sampling times and sensitive bioanalytical methods. According to the prescribing information for sublingual buprenorphine (Suboxone® and Subutex®), “buprenorphine has a mean elimination half-life from plasma of 37 h”. The elimination half-life of its principal metabolite, norbuprenorphine, is even longer, at approximately 57 hours (Kuhlman et al, Addiction 1998; 93:549-559). In addition, buprenorphine has a high affinity and slow dissociation from the μ-opioid receptor. These attributes provide sublingual buprenorphine with a long duration of therapeutic effect, allowing for good therapeutic outcomes when given once-a-day, once every two days and thrice weekly by the sublingual route (Kuhlman et al, Addiction 1998; 93:549-559; Amass et al, Drug and Alcohol Dependence, 2000; 58:143-52; Amass et al, Drug and Alcohol Dependence, 2001; 61:173-81; Marsch et al, Drug and Alcohol Dependence, 2005; 77:195-204; Fudala et al, Clin Pharmacol Ther 1990; 47:525-34; de los Cobos et al, Drug and Alcohol Dependence 200; 59:223-233). According to the manufacturer, “Suboxone works best when taken every 24 hours; however, it may last longer than 24 hours, so you may not get sick” (http://www.suboxone.com/pdfs/FAQsPatients.pdf). Contrary to this, it has now been discovered that when given by the oral route, the duration of therapeutic effect for the treatment of pain is considerably less than 24 hours, less than 12 hours, or less than 8 hours.

There is therefore a need for new pharmaceutical compositions and methods for the treatment of pain, opioid dependence, addiction disorders, cough, dyspnea and other buprenorphine responsive medical conditions which provide a prolonged duration of therapeutic effect (e.g., more than 8 hours, preferably more than 12 hours, most preferably at least about 14 hours, 16 hours, 18 hours, 20 hours or 24 hours) when given orally.

There is therefore also a need for new controlled release, delayed onset, duodenal release, jejunal release, ileal release, ileo-colonic release and colonic release pharmaceutical compositions and methods for the treatment of pain, opioid dependence, addiction disorders, cough, dyspnea and other buprenorphine responsive medical conditions which provide a prolonged duration of therapeutic effect (e.g., more than 8 hours, preferably more than 12 hours, most preferably at least about 14 hours, 16 hours, 18 hours, 20 hours or 24 hours) when given orally.

There is therefore a need for new pharmaceutical compositions and methods for the treatment of pain, opioid dependence, addiction disorders, cough, dyspnea and other buprenorphine responsive medical conditions which provide a prolonged duration of therapeutic effect when given orally, such that it may be administered orally about every two or three days.

A pharmaceutically acceptable dosage form of oral buprenorphine for the treatment of buprenorphine responsive conditions, including pain and opioid addiction disorders beyond its now discovered short duration of action at a controlled rate over an extended period of time appears to be lacking in the pharmaceutical and medical arts.

In view of the foregoing presentation, it is immediately apparent that a serious need exists for an improvement in the delivery of oral buprenorphine for its therapeutic effect. The need exists to provide a novel therapeutic composition comprising oral buprenorphine, the need exists to provide a novel dosage form comprising oral buprenorphine, and the need to provide a novel method of administering buprenorphine to a patient in need of buprenorphine therapy. The invention provides an oral, relatively easy mode and manner of buprenorphine administration in comparison with sublingual, transdermal, intranasal and parenteral administration.

A widely used commercially available sublingual formulation of contains buprenorphine in combination with naloxone in an optimized ratio of 4 parts buprenorphine base to 1 part naloxone base)(Suboxone®. Contrary to this ratio, in some embodiments, the orally administered buprenorphine pharmaceutical compositions of the invention are optimized with a significantly lower amount of naloxone base relative to buprenorphine base to provide an optimal balance of efficacy and safety and abuse deterrence.

To the applicants knowledge, no examples or working prototype formulations of any modified release oral buprenorphine have been described in the prior art. Buprenorphine is a challenging drug to develop orally due to its high intrinsic clearance and the potential for substantial pharmacokinetic variability.

To the applicant's knowledge, there are no data heretofore demonstrating the efficacy of any oral pharmaceutical compositions of buprenorphine for any buprenorphine responsive medical conditions.

To the applicant's knowledge, there are no data demonstrating the efficacy of any oral immediate release pharmaceutical compositions of buprenorphine for the treatment of any buprenorphine responsive medical conditions, including acute pain, chronic pain, cancer pain, neuropathic pain, opioid dependence, dyspnea, cough and addiction disorders.

To the applicant's knowledge, there are no data demonstrating the efficacy of any oral sustained release pharmaceutical compositions of buprenorphine for the treatment of any buprenorphine responsive medical conditions, including acute pain, chronic pain, cancer pain, neuropathic pain, opioid dependence, dyspnea, cough and addiction disorders.

To the applicant's knowledge, there are no data demonstrating the efficacy of any delayed onset, duodenal delivery, jejunal delivery, ileal delivery, ileo-colonic delivery or colonic delivery dosage forms of oral buprenorphine for the treatment of any buprenorphine responsive medical conditions, including acute pain, chronic pain, cancer pain, neuropathic pain, opioid dependence, dyspnea, cough and addiction disorders.

There is a need for new pharmaceutical compositions and methods for the treatment of pain, opioid dependence, addiction disorders and buprenorphine or opioid responsive medical conditions.

There is a need for new oral opioid pharmaceutical compositions and methods for the treatment of pain, opioid dependence, addiction disorders and buprenorphine or opioid responsive medical conditions have high efficacy, good tolerability, are an alternative to the transdermal and sublingual/buccal routes, and provide consistent pharmacokinetics and pharmacodynamics.

### DESCRIPTION OF THE INVENTION

The present invention is directed at oral buprenorphine pharmaceutical compositions and methods for the treatment of pain, addiction disorders, dyspnea and cough.

The present invention is also directed at oral pharmaceutical compositions of buprenorphine and their use for the treatment of other buprenorphine or opioid responsive medical conditions.

In some preferred embodiments, the oral pharmaceutical compositions of the invention are delayed onset dosage forms of buprenorphine.

In some preferred embodiments, the oral pharmaceutical compositions of the invention are modified release (e.g., controlled release, delayed onset, extended release, sustained release, slow release) dosage forms of buprenorphine.

In some preferred embodiments, the oral pharmaceutical compositions of the invention are delayed onset, rapid release dosage forms of buprenorphine.

In some preferred embodiments, the oral pharmaceutical compositions of the invention are delayed onset, extended release dosage forms of buprenorphine.

In some preferred embodiments, the oral pharmaceutical compositions of the invention are delayed onset, pulsatile release dosage forms of buprenorphine.

In some preferred embodiments, the oral pharmaceutical compositions of the invention are delayed onset, rapid release; delayed onset, extended release; or delayed onset, pulsatile release; or duodenal release; or jejunal release; or ileal release; or ileo-colonic release; or colonic release.

In some embodiments, the oral pharmaceutical compositions of the invention comprise immediate release buprenorphine.

The present invention also provides advantages in that equivalent, or higher doses of buprenorphine may be used with better efficacy and/or fewer side effects observed than with sublingual formulations. For example, oral buprenorphine formulations of the present invention may include the same amount or up to 5-fold higher daily maximum doses of sublingual buprenorphine. However, even with these higher doses, formulations of the present invention achieve better efficacy and fewer side effects.

The present invention is directed at oral pharmaceutical composition for the treatment of pain, opioid dependence, addiction disorders and other buprenorphine and opioid responsive medical conditions comprising a therapeutically effective amount of buprenorphine or a pharmaceutically acceptable salt of buprenorphine, or a mixture thereof, said dosage form not intended to provide significant oro-mucosal, lingual, sublingual or buccal absorption, said dosage form not intended for oro-mucosal, lingual, sublingual or buccal administration.

In some embodiments, the present invention is directed at oral pharmaceutical compositions of buprenorphine which are stable without the need to incorporate antioxidants or chelating agents into the dosage form to provide stability to the buprenorphine API.

In some embodiments, the present invention is directed at oral pharmaceutical compositions of buprenorphine which are stable without the need for antioxidants or chelating agents in direct or substantial contact with the buprenorphine API.

In some embodiments, the present invention is directed at pharmaceutical compositions of buprenorphine which are pharmaceutically and therapeutically robust when given orally and which release the buprenorphine distal to the stomach, duodenum, jejunum or ileum.

Surprisingly, oral administration of buprenorphine can produce robust, dose dependent therapeutic effects.

Surprisingly, oral administration of buprenorphine delivered to the ileum or colon produces a robust therapeutic effect; said effect greater that oral immediate release buprenorphine delivered to the stomach.

Surprisingly, oral administration of buprenorphine in a dosage form that provides controlled release; or extended release; or sustained release; or modified release; or delayed onset, rapid release; or delayed onset, extended release; or delayed onset, pulsatile release; or duodenal release; or jejunal release; or ileal release; or ileo-colonic release; or colonic release of buprenorphine provides a significantly more robust therapeutic effects than following oral immediate release dosage forms.

Surprisingly, oral dosage forms of the invention provide robust stability without the need for anti-oxidants or chelating agents to stabilize the buprenorphine.

Surprisingly, oral dosage forms of the invention that provide controlled release; extended release; sustained release; modified release; delayed onset, rapid release; delayed onset, extended release; delayed onset, pulsatile release; duodenal release; jejunal release; ileal release; ileo-colonic release; or colonic release of buprenorphine are highly stable without the need for anti-oxidants or chelating agents to stabilize the buprenorphine.

Buprenorphine has been found to degrade upon stress degradation in acid and H₂O₂ (oxidation), each producing impurities which are known to the applicant (oxidative stress) and impurities which are unknown to the applicant (oxidative stress and acid stressed). The, applicant has found that the behavior of oral buprenorphine dosage forms incorporating certain widely used controlled release material results in unexpected deterioration in the dissolution rate of buprenorphine under acidic conditions (increase in release at pH 1.2), providing a rationale in some embodiments of the invention for protection of the dosage form from the acid media of the stomach and release from the dosage form at some point distal to the stomach.

Surprisingly, oral administration of buprenorphine is more tolerable and produced fewer and less intense common opioid side effects compared with sublingual buprenorphine.

Although buprenorphine has a long elimination half-life, and high affinity and slow dissociation from the μ-opioid receptor which provide good therapeutic outcomes when given once-a-day, once every two days and thrice weekly in immediate release form by the sublingual route, oral administration of an immediate release form of buprenorphine unexpectedly provides a duration of therapeutic effect less than about 24 hours, or less than about 18 hours, or less than about 16 hours, or less than about 14 hours, or less than about 12 hours, or less than about 9 hours, or less than about 8 hours, or less than about 7 hours, or less than about 6 hours.

In some embodiments, such an unexpectedly short duration of provides a basis for a controlled release, delayed onset, extended release, or delayed onset, pulsatile release, buprenorphine oral dosage form of the invention and methods for the treatment of pain, opioid dependence, addiction disorders, cough, dyspnea and other buprenorphine responsive medical conditions which result in a prolonged duration (e.g., equal to or more than about 6, 7, 8, 9, 10, 12, 14, 16, 18 or 22, 23 or 24 hours) of therapeutic effect (e.g., pain relief).

In some embodiments, the controlled release, delayed onset, extended release, or delayed onset, pulsatile release duodenal release, jejunal release, ileal release, ileo-colonic release or colonic release buprenorphine oral dosage form of the invention provide a sufficiently prolonged duration of therapeutic effect so that it may be administered about every 36 hours, or about every 48 hours or about every 60 hours, or about every 72 hours or about every 84 hours.

In some preferred embodiments, the oral pharmaceutical compositions of the invention provide greater dose proportional bioavailability over a 2, 3, 4, 5, 6, 7, 8, 9, 10 and 15-fold dose range than sublingual or nasal formulations of buprenorphine.

Although the commercially available sublingual formulation of buprenorphine in combination with naloxone contain an optimized ratio of 4 parts buprenorphine base to 1 part naloxone base (Suboxone®), in some embodiments, the dosage form of the invention is optimized with a lower amount of naloxone base relative to buprenorphine base to provide an optimal balance of efficacy, safety and abuse deterrence (e.g., well tolerated when taken orally as prescribed, and aversive when abused without placing the illicit drug user at serious risk). In some embodiments, by increasing the ratio of buprenorphine to naloxone in the oral dosage form of the invention (i.e., lower amount of naloxone base relative to buprenorphine base), the: (i) tolerability and safety of the formulation can be improved in patients taking the drug as prescribed (e.g., reduced potential for diarrhea, nausea, symptoms of opioid withdrawal and abstinence); (ii) efficacy can be improved (e.g., reduced potential for pain and positive urine samples for illicit drugs); and (iii) pharmacokinetics and absorption profile of buprenorphine can be improved (e.g., reduced hypermotility and more reproducible gastrointestinal transit time), particularly for dosage forms of the invention which are controlled release; delayed onset, rapid release; delayed onset, extended release; or delayed onset, pulsatile release; or duodenal release; r jejunal release; or ileal release; or ileo-colonic release; or colonic release). In addition, by reducing the ratio of buprenorphine to naloxone in the oral dosage form of the invention, the cost of goods is also reduced.

In some embodiments, the ratio of buprenorphine base to naloxone base is more than about: 6:1, 7:1, 8:1, 9:1, 10:1, 12:1, 14:1, 16:1, 18:1, 20:1, 22:1, 24:1, 26:1, 28:1, or 30:1.

In some embodiments, the ratio of naloxone may be replaced with naltrexone, and the buprenorphine base to naltrexone base ratio is more than about: 6:1, 7:1, 8:1, 9:1, 10:1, 12:1, 14:1, 16:1, 18:1, 20:1, 22:1, 24:1, 26:1, 28:1, 30:1; 35:1; 40:1; 45:1 50:1; 55:1, 60:1, 65:1, 70:1, 75:1, 80:1, 85:1 or 90:1.

In some embodiments, the present invention is directed to oral pharmaceutical compositions of buprenorphine comprising naloxone, where the ratio of systemic exposure to naloxone as measured by the area under the plasma naloxone concentration-time curve from time 0 to 48 hours or 0 to infinity (AUC₀₋₄₈ or AUC_{0-inf}) after single dose administration of lingual, sublingual or buccal buprenorphine to the oral buprenorphine dosage form of the invention is at least about 2:1, 3:1, 4; 1, 5:1, 6:1, 7:1, 8:1, 9:1, 10:1, 12:1, 14:1, 16:1, 18:1, 20:1, 22:1, 24:1, 26:1, 28:1, or 30:1, after the same amount of naloxone is administered.

In some embodiments, the present invention is directed to oral pharmaceutical compositions of buprenorphine comprising naloxone, where the ratio of systemic exposure to naloxone as measured by the area under the plasma naloxone concentration-time curve from time 0 to 48 hours or 0 to infinity (AUC₀₋₄₈ or AUC_{0-inf}) after single dose administration of lingual, sublingual or buccal naltrexone to the oral buprenorphine dosage form of the invention comprising naloxone is at least about 2:1, 3:1, 4; 1, 5:1, 6:1, 7:1, 8:1, 9:1, 10:1, 12:1, 14:1, 16:1, 18:1, 20:1, 22:1, 24:1, 26:1, 28:1, or 30:1, after the same amount of naloxone is administered.

In some embodiments, the present invention is directed to oral pharmaceutical compositions of buprenorphine comprising naltrexone, where the ratio of systemic exposure to naltrexone as measured by the area under the plasma naloxone concentration-time curve from time 0 to 48 hours or 0 to infinity (AUC₀₋₄₈ or AUC_{0-inf}) after single dose administration of lingual, sublingual or buccal buprenorphine comprising naltrexone to the oral buprenorphine dosage form of the invention comprising naltrexone is at least about 2:1, 3:1, 4; 1, 5:1, 6:1, 7:1, 8:1, 9:1, 10:1, 12:1, 14:1, 16:1, 18:1, 20:1, 22:1, 24:1, 26:1, 28:1, or 30:1, after the same amount of naltrexone is administered.

In some embodiments, the present invention is directed to oral pharmaceutical compositions of buprenorphine comprising naltrexone, where the ratio of systemic exposure to naltrexone as measured by the area under the plasma naloxone concentration-time curve from time 0 to 48 hours or 0 to infinity (AUC₀₋₄₈ or AUC_{0-inf}) after single dose administration of lingual, sublingual or buccal naltrexone to the oral buprenorphine dosage form of the invention comprising naltrexone is at least about 2:1, 3:1, 4; 1, 5:1, 6:1, 7:1, 8:1, 9:1, 10:1, 12:1, 14:1, 16:1, 18:1, 20:1, 22:1, 24:1, 26:1, 28:1, or 30:1, after the same amount of naltrexone is administered.

In some embodiments, the present invention is directed to oral pharmaceutical compositions of buprenorphine comprising naloxone, where the ratio of peak plasma naloxone concentration (Cₘₐₓ) after single dose administration of lingual, sublingual or buccal buprenorphine to the oral buprenorphine dosage form of the invention is at least about 2:1, 3:1, 4; 1, 5:1, 6:1, 7:1, 8:1, 9:1, 10:1, 12:1, 14:1, 16:1, 18:1, 20:1, 22:1, 24:1, 26:1, 28:1, or 30:1, after the same amount of naloxone is administered.

In some embodiments, the present invention is directed to oral pharmaceutical compositions of buprenorphine comprising naloxone, where the ratio of peak plasma naloxone concentration (Cₘₐₓ) after single dose administration of lingual, sublingual or buccal naltrexone to the oral buprenorphine dosage form of the invention comprising naloxone is at least about 2:1, 3:1, 4; 1, 5:1, 6:1, 7:1, 8:1, 9:1, 10:1, 12:1, 14:1, 16:1, 18:1, 20:1, 22:1, 24:1, 26:1, 28:1, or 30:1, after the same amount of naloxone is administered.

In some embodiments, the present invention is directed to oral pharmaceutical compositions of buprenorphine comprising naltrexone, where the ratio of peak plasma naltrexone concentration (Cₘₐₓ) after single dose administration of lingual, sublingual or buccal buprenorphine comprising naltrexone to the oral buprenorphine dosage form of the invention comprising naltrexone is at least about 2:1, 3:1, 4; 1, 5:1, 6:1, 7:1, 8:1, 9:1, 10:1, 12:1, 14:1, 16:1, 18:1, 20:1, 22:1, 24:1, 26:1, 28:1, or 30:1, after the same amount of naltrexone is administered.

In some embodiments, the present invention is directed to oral pharmaceutical compositions of buprenorphine comprising naltrexone, where the ratio of peak plasma naltrexone concentration (Cₘₐₓ) after single dose administration of lingual, sublingual or buccal naltrexone to the oral buprenorphine dosage form of the invention comprising naltrexone is at least about 2:1, 3:1, 4; 1, 5:1, 6:1, 7:1, 8:1, 9:1, 10:1, 12:1, 14:1, 16:1, 18:1, 20:1, 22:1, 24:1, 26:1, 28:1, or 30:1, after the same amount of naltrexone is administered.

In some embodiments, the present invention is directed to oral pharmaceutical compositions of buprenorphine comprising naloxone, where the systemic exposure to naloxone as measured by the area under the plasma naloxone concentration-time curve from time 0 to 48 hours or 0 to infinity (AUC₀₋₄₈ or AUC_{0-inf}) after single dose administration, for each mg of naloxone in the oral dosage form, is less than about: 400 pg·hr/mL, 350 pg·hr/mL, 300 pg·hr/mL, 250 pg·hr/mL, 200 pg·hr/mL, or 150 pg·hr/mL, or 100 pg·hr/mL, 75 pg·hr/mL, or 50 pg·hr/mL, or 40 pg·hr/mL, or 30 pg·hr/mL, or 25 pg·hr/mL, or 20 pg·hr/mL, or 15 pg·hr/mL, or 10 pg·hr/mL, or 7 pg·hr/mL, or 5 pg·hr/mL, or 2.5 pg·hr/mL, or 1 pg·hr/mL,

In some embodiments, the present invention is directed to oral pharmaceutical compositions of buprenorphine comprising naltrexone, where the systemic exposure to naltrexone as measured by the area under the plasma naltrexone concentration-time curve from time 0 to 48 hours or 0 to infinity (AUC₀₋₄₈ or AUC_{0-inf}) after single dose administration, for each mg of naltrexone in the oral dosage form, is less than about: 400 pg·hr/mL, 350 pg·hr/mL, 300 pg·hr/mL, 250 pg·hr/mL, 200 pg·hr/mL, or 150 pg·hr/mL, or 100 pg·hr/mL, 75 pg·hr/mL, or 50 pg·hr/mL, or 40 pg·hr/mL, or 30 pg·hr/mL, or 25 pg·hr/mL, or 20 pg·hr/mL, or 15 pg·hr/mL, or 10 pg·hr/mL, or 7 pg·hr/mL, or 5 pg·hr/mL, or 2.5 pg·hr/mL, or 1 pg·hr/mL.

In some embodiments, the present invention is directed to oral pharmaceutical compositions of buprenorphine comprising naloxone, where the peak plasma naloxone concentration (Cₘₐₓ) after single dose administration, for each mg of naloxone in the oral dosage form, is less than about: 80 pg/mL, 70 pg/mL, 60 pg/mL, 50 pg/mL, 40 pg/mL, or 30 pg/mL, or 20 pg/mL, 10 pg/mL, or 8 pg/mL, or 6 pg/mL, or 5 pg/mL, or 3 pg/mL, or 2 pg/mL, or 1 pg/mL.

In some embodiments, the present invention is directed to oral pharmaceutical compositions of buprenorphine comprising naltrexone, where the peak plasma naltrexone concentration (Cₘₐₓ) after single dose administration, for each mg of naltrexone in the oral dosage form, is less than about: 80 pg/mL, 70 pg/mL, 60 pg/mL, 50 pg/mL, 40 pg/mL, or 30 pg/mL, or 20 pg/mL, 10 pg/mL, or 8 pg/mL, or 6 pg/mL, or 5 pg/mL, or 3 pg/mL, or 2 pg/mL, or 1 pg/mL.

In some embodiments, the oral buprenorphine dosage forms of the invention comprising naloxone or naltrexone in the amounts, ratios or exposure level in the specifications are therapeutically effective and without unacceptable side effects in most or substantially all subjects taking it as medically prescribed

In particularly preferred embodiments, the oral buprenorphine dosage form of the invention comprises naltrexone or naloxone, said dosage having a buprenorphine base to naltrexone base or naloxone base ratio of more than about: 6:1 to about 12:1.

In particularly preferred embodiments, the oral buprenorphine dosage form of the invention comprises naltrexone or naloxone, said dosage form providing an AUC₀₋₄₈ or AUC_{0-inf} after single dose administration, for each mg of naltrexone base or naloxone base in the oral dosage form of less than about 300 pg·hr/mL or less than about 100 pg·hr/mL.

In particularly preferred embodiments, the oral buprenorphine dosage form of the invention comprises naltrexone or naloxone, said dosage form providing a naltrexone or naloxone Cₘₐₓ after single dose administration for each mg of naltrexone base or naloxone base in the oral dosage of less than about 50 pg/mL or less than about 10 pg/mL.

In some embodiments, the oral buprenorphine dosage forms of the invention comprising naloxone or naltrexone in the amounts, ratios or exposure level in the specifications are abuse deterrent in some, most, substantially all or all recreational opioid users and opioid abusers when the dosage form is tampered with and the contents (buprenorphine plus naloxone or buprenorphine plus naltrexone) are injected.

In some embodiments, the invention provides an oral dosage form for the treatment of buprenorphine responsive conditions requiring chronic buprenorphine therapy which is designed to improve treatment compliance and deter episodic, occasional, intermittent, periodic, as needed, or PRN use of the dosage form by rendering the dosage form therapeutically ineffective or suboptimally effective when taken episodically, intermittently, occasionally, periodically, on an as needed basis, or PRN, said dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release when given orally.

In some embodiments, the invention provides an oral dosage form for the treatment of buprenorphine responsive conditions requiring around the clock or time contingent buprenorphine therapy which is designed to improve treatment compliance and deter episodic, occasional, intermittent, periodic, as needed, or PRN use of the dosage form by rendering the dosage form therapeutically ineffective or suboptimally effective when taken episodically, intermittently, occasionally, periodically, on an as needed basis, or PRN, said dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release when given orally.

In some embodiments, the invention provides an oral dosage form of buprenorphine which is therapeutically effective but which deters the abuse and misuse of the dosage form by rendering the dosage devoid of or substantially devoid of euphoria, pleasurable effects, drug liking and mood alerting effects when taken episodically, intermittently, occasionally, periodically, on an as needed basis, or PRN, said dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release when given orally.

In some embodiments, the invention provides an oral dosage form of buprenorphine which is therapeutically effective but which deters the abuse and misuse of the dosage form by delaying or substantially delaying euphoria, pleasurable effects, drug liking and mood alerting effects when taken regularly or when taken episodically, intermittently, occasionally, periodically, on an as needed basis, or PRN, said dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release when given orally.

In some embodiments, the invention provides a method of improving treatment compliance and deterring episodic, occasional, intermittent, periodic, as needed, or PRN use of the dosage form in subjects requiring around the clock, scheduled or time contingent oral buprenorphine therapy by rendering the dosage form therapeutically ineffective or suboptimally effective when taken episodically, intermittently, occasionally, periodically, on an as needed basis, or PRN, said dosage form therapeutically effective in a substantial number of subjects when taken around the clock, on scheduled basis or on time contingent basis, comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release when given orally.

In some embodiments, the invention provides a method of improving treatment compliance and deterring episodic, occasional, intermittent, periodic, as needed, or PRN use of the dosage form in subjects requiring chronic or prolonged oral buprenorphine therapy by rendering the dosage form therapeutically ineffective or suboptimally effective when taken episodically, intermittently, occasionally, periodically, on an as needed basis, or PRN, said dosage form therapeutically effective in a substantial number of subjects when taken around the clock, on scheduled basis or on time contingent basis, comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release when given orally.

In some embodiments, the invention provides a method of deterring abuse and misuse of oral buprenorphine by rendering the dosage form devoid of or substantially devoid of euphoria, pleasurable effects, drug liking and mood alerting effects when taken episodically, intermittently, occasionally, periodically, on an as needed basis, or PRN, said dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release when given orally.

In some embodiments, the invention provides a method of deterring abuse and misuse of oral buprenorphine by delaying or substantially delaying or reducing euphoria, pleasurable effects, drug liking and mood alerting effects when taken regularly or when taken episodically, intermittently, occasionally, periodically, on an as needed basis, or PRN, said dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release when given orally.

In some embodiments, the invention provides an oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release, said dosage form providing less side effects than sublingual buprenorphine, said side effects measured after single dose administration to opioid naïve subjects.

In some embodiments, the invention provides an oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release, said dosage form providing less side effects than oral immediate release buprenorphine, said side effects measured after single dose administration to opioid naïve subjects.

In some embodiments, the invention provides a method for reducing side effects, comprising administering an oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing less side effects than sublingual buprenorphine, said side effects measured after single dose administration to opioid naïve subjects.

In some embodiments, the invention provides a method for reducing side effects, comprising administering an oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing less side effects than oral immediate release buprenorphine, said side effects measured after single dose administration to opioid naïve subjects.

In some embodiments, the present invention is directed to oral pharmaceutical compositions of buprenorphine which provide one or more of the following (i) efficacy or improved efficacy by the oral route; (ii) improved safety by the oral route; (iii) improved efficiency of the dosage form; (iv) improved dose proportional extent of absorption; (v) reduced variability in absorption; and (vi) reduced potential for misuse, abuse, addiction and drug diversion. Without being bound by theory, in some embodiments, the pharmaceutical compositions and methods of the invention achieve their objectives at least in part through delivery of some, most, substantially all or all of the drug into the lower segments of the gastrointestinal tract (e.g., delayed onset, rapid release; or delayed onset, extended release; or delayed onset, pulsatile release; or duodenal release; or jejunal release; or ileal release; or ileo-colonic release; or colonic release) through time-controlled, pH-controlled, pressure-controlled, enzyme-controlled and hydration controlled.

In some embodiments, the oral pharmaceutical compositions of buprenorphine of the present invention improve the clinical efficacy, safety, tolerability, abuse resistance, improved dose proportional extent of absorption, predictability of clinical response, reproducibility of clinical response, efficiency, and/or pharmacokinetics, said effect achieved through targeted gastrointestinal delivery, availability, release, disintegration, dissolution, metabolism and/or absorption, said targeted delivery achieved at least in part through delivery of some, most, substantially all or all of the drug in lower segments of the gastrointestinal tract (e.g., delayed onset, rapid release; or delayed onset, extended release; or delayed onset, pulsatile release; or duodenal release; or jejunal release; or ileal release; or ileo-colonic release; or colonic release) by incorporation of materials into the dosage form to provide time-controlled, diffusion-controlled pH-controlled, pressure-controlled, osmotic pressure controlled and/or enzyme controlled delivery or release.

In some embodiments, the oral controlled release; or delayed onset, rapid release; or delayed onset, extended release; or delayed onset, pulsatile release; or duodenal release; or jejunal release; or ileal release; or ileo-colonic release; or colonic release pharmaceutical compositions of buprenorphine result in one or more or all of the following benefits or characteristics when given at equal doses compared with immediate release oral dosage forms: increased dose proportional extent of absorption; increased peak concentration (Cₘₐₓ); increased time to peak concentration (tₘₐₓ); reduced variability in extent of absorption (% coefficient of variation for AUC_{0-inf} or AUC₀₋₁); reduced apparent oral clearance; lower norbuprenorphine to buprenorphine AUC_{0-inf} ratio; improved clinical efficacy; improved safety; reduced side effects 0.5, 1, 2, 3, 4 5, 6, 7 and/or 8 hours after oral ingestion of the dose; reduced abuse potential in drug abusers and recreational drug users; reduced abuse potential in subjects using the drug in accordance with the instructions of the medical practitioner, manufacturer, approved prescribing information or package insert; reduced risk of drug diversion, addiction or iatrogenic addiction; more predictable or reproducible clinical response; more efficient clinical response; more predictable or reproducible analgesic; and more efficient analgesic response.

In some embodiments, the oral controlled release; or delayed onset, rapid release; or delayed onset, extended release; or delayed onset, pulsatile release; or duodenal release; or jejunal release; or ileal release; or ileo-colonic release; or colonic release pharmaceutical compositions of buprenorphine liberate or deliver some, a lot, most, substantially all or all of the buprenorphine in the dosage form upon reaching the duodenum, terminal ileum, ileo-cecal junction, ascending colon, transverse colon or descending colon.

In some embodiments, the oral controlled release; or delayed onset, rapid release; or delayed onset, extended release; or delayed onset, pulsatile release; or duodenal release; or jejunal release; or ileal release; or ileo-colonic release; or colonic release pharmaceutical compositions of buprenorphine start to liberate or deliver some, a lot, most, substantially all or all of the buprenorphine in the dosage form upon reaching the duodenum, terminal ileum, ileo-cecal junction, ascending colon, transverse colon or descending colon.

An important drawback with the use of buprenorphine is the risk of drug addiction, drug diversion and drug abuse. Although the use of opioids for non-medical purposes has existed throughout recorded human history, their abuse has increased significantly in the past two decades (Drug Abuse Warning Network, http://dawninfo.samhsa.gov/; Drug Enforcement Administration, http://www.deadiversion.usdoj.gov/; National Survey on Drug Use & Health, http://www.oas.samhsa.gov/nhsda.htm; American Association of Poison Control Centers Toxic Exposure Surveillance System, http://www.aapcc.org/annual.htm).

Our increased understanding of the clinical pharmacology of opioids and data from well controlled clinical trials in chronic non-cancer pain (Peloso et al., Journal of Rheumatology 2000; 27:764-71; Caldwell, et al., Journal of Pain and Symptom Management 2002; 23:278-91; Matsumoto et al., Pain Medicine 2005; 6:357-66; Arkinstall et al., Pain 1995; 62:169-78) and neuropathic pain (Watson and Babul, Neurology 1998; 50:1837-41) have resulted in more widespread use in patients with non-malignant pain (for a review, see Sloan and Babul, Expert Opinion on Drug Delivery 2006; 3:489-97). This in turn has led to concerns about the increased non-medical use of opioids through both licit and illicit channels. For instance, unsuspecting clinicians may prescribe buprenorphine for pain to individuals with an addiction disorder or individuals with pain who divert a portion of their prescribed dose to other individuals. Alternatively, clinicians may prescribe buprenorphine to treat individuals with an addiction disorder who divert a portion of their prescribed dose to other individuals. There have also been documented cases of inappropriate prescribing or dispensing of opioids by physicians and pharmacists, with its eventual diversion into the non-medical marketplace. Additionally, non-medical supplies of pharmaceutical grade buprenorphine may be obtained through prescription forgeries and break-ins into pharmacies.

Pharmaceutical dosage forms containing buprenorphine have been abused in a variety of settings, including: (i) patients with an addiction disorder who are prescribed buprenorphine for opioid maintenance therapy but who are using it in excess doses or by an unapproved route; (ii) patients with an addiction disorder who obtain it from illicit sources; (iii) patients with pain who have a pre-existing addiction disorder; (iv) patients with pain who have developed an addiction disorder following initiation of buprenorphine or opioid therapy; (v) recreational drug users.

Abused opioid analgesics may be ingested whole, crushed and ingested, crushed or vaporized and snorted or injected intravenously after attempted extraction of the active pharmaceutical ingredient. The manipulation of pharmaceutical dosage forms of opioids has been documented for many decades. For instance, pentazocine (Talwin®), a synthetic opioid was crushed, extracted and injected intravenously by drug addicts.

In a many cases, the abuse of pharmaceutical grade opioids in finished dosage forms is in its intact form (i.e., the dosage form has not been physically manipulated or tampered with to alter its absorption profile) and taken by the usual route of administration for that dosage form. For example, many recreational drug users and patients with an addiction disorder will not use an abusable drug intended to be taken by the oral route by any other route (e.g., intravenously after extraction and filtration, or by inhalation), nor will they physically manipulate or tamper the dosage form prior to oral ingestion, in order to “distinguish” themselves and their use from “junkies’ and “real addicts”.

In a many cases, opioid abuse by the oral route involves immediate release drugs (i.e., drugs that have not been designed with material to delay the liberation and absorption of the opioid from the dosage form) or drugs in which such material has been tampered. Such immediate release opioids generally provide meaningful plasma concentrations, an onset of therapeutic effect (e.g., pain relief or blunting of opioid withdrawal symptoms), and in the case of use by recreational drug users or addicts, an onset of euphoric or psychic within about 15 to about 180 minutes or within about 15 to about 120 minutes or within about 15 to about 90 minutes.

For an opioid agonist to have appeal as an abusable drug amongst recreational drug users or addicts for other misuse (e.g, used outside the usage parameters or conditions specified by the clinician or outside the approved prescribing information), it must produce rapid onset of euphoric or psychic effects.

Unfortunately, when given orally to recreational drug users or addicts, many opioid produce perceptible or meaningful euphoric or psychic effects soon after administration, usually within about 15 to about 180 minutes or within about 15 to about 120 minutes or within about 15 to about 90 minutes, even when taken in an untampered form.

A temporal relationship between drug ingestion and the appearance of discernible euphoric or psychic effects is essential to its abuse by recreational drug users or addicts and to its high “street value” amongst recreational drug users or addicts.

Although there has been considerable discussion in both the lay and medical the press about the “popularity” of extended release oxycodone among addicts and recreational drug users for non-medical use, its use in a tampered form (including crushing, pulverizing, solvent extraction, intravenous administration), and methods to prevent such tampering, a significant amount of abuse of the extended release dosage form of oxycodone and other extended release opioids is by oral administration of the intact dosage form alone or with other drugs of abuse or alcohol.

Extended release opioids generally provide peak plasma concentrations soon after ingestion, for example, approximately 1.5 hours after MS Contin® and approximately 2 to 3 hours after OxyContin®. Although their peak plasma concentration is generally somewhat later than oral immediate release opioid analgesics, the 12 or 24-hour supply of opioid contained in one tablet or capsule, means that their plasma concentration after ingestion is generally similar to that provided by a 4 to 6 hourly dose of an immediate release formulation. Other extended release opioids with delayed time to peak concentrations continue to provide meaningful plasma concentrations soon after ingestion, even if the maximum concentration is delayed. Therefore, when given orally in an untampered form, all commercially available extended release opioid analgesics are associated with the appearance of discernible euphoric or psychic effects within about 15 to about 180 minutes or within about 15 to about 120 minutes or within about 15 to about 90 minutes.

Unfortunately, when given orally in an untampered form, extended release opioids produce perceptible or even meaningful euphoric or psychic effects soon after administration, usually within about 15 to about 180 minutes or within about 15 to about 120 minutes or within about 15 to about 90 minutes.

By delaying the release and subsequent absorption of some, most, substantially all or all of the buprenorphine from the dosage form, in some embodiments, the dosage form of the invention will provide discernible euphoric or psychic effects at a later time by delaying the time to attainment of pharmacologically discernible or meaningful plasma concentrations or by providing a lower Cₘₐₓ and/or later tₘₐₓ than oral, immediate release dosage forms containing the same drug and oral, extended release dosage forms containing the same drug. Consequently, in some embodiments, the dosage forms of the invention will have a lower propensity for abuse and misuse.

In some embodiments, the buprenorphine dosage forms of the invention have a reduced potential for abuse and misuse, including: (a) drug abuse in individuals with a history of drug abuse or recreational drug use; (b) drug abuse in individuals with no prior history of drug abuse or recreational drug use; (c) iatrogenic addiction, euphoria, or pleasurable effects in individuals who take the dosage form in accordance with the instructions of the clinician, approved prescribing information or approved package insert; (d) drug diversion; (e) pharmaceutical company liability; (f) pharmacy break-ins, prescription forgeries, doctor shopping; (g) illicit (street) availability and price; and/or (g) mood altering effects, said reduced potential abuse and misuse resulting in some embodiments at least in part from introducing a substantial lag period between oral ingestion of the buprenorphine and the appearance of detectable plasma concentrations, or clinically meaningful plasma concentrations, or pharmacologically meaningful plasma concentrations; or pharmacologically discernible plasma concentrations, or plasma concentrations associated with CNS reinforcing effects, or plasma concentrations associated with mood altering effects.

In some embodiments, the present invention is directed to oral dosage forms of buprenorphine for the treatment of buprenorphine responsive conditions requiring chronic buprenorphine therapy where the dosage form is designed to improve treatment compliance and deter episodic, occasional, intermittent, periodic, as needed, or PRN use by rendering the dosage form therapeutically ineffective or suboptimally effective when taken episodically, intermittently, occasionally, periodically, on an as needed basis, or PRN, said dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release. In some embodiments, said dosage form improves compliance or deters episodic, occasional, intermittent, periodic, as needed, or PRN use by more than about 1%, or 2%, or 3%, or 5%, or 7%, or 10%, or 12%, or 15%, or 18%, or 20%, or 25%, or 30%, or 35%, or 40%, or 50%, or 60%, or 70%, or 100%.

In some embodiments, the present invention is directed to oral dosage forms of buprenorphine for the treatment of buprenorphine responsive conditions around the clock or time contingent buprenorphine therapy where the dosage form is designed to improve treatment compliance and deter episodic, occasional, intermittent, periodic, as needed, or PRN use by rendering the dosage form therapeutically ineffective or suboptimally effective when taken episodically, intermittently, occasionally, periodically, on an as needed basis, or PRN, said dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release. In some embodiments, said dosage form improves compliance or deters episodic, occasional, intermittent, periodic, as needed, or PRN use by more than about 1%, or 2%, or 3%, or 5%, or 7%, or 10%, or 12%, or 15%, or 18%, or 20%, or 25%, or 30%, or 35%, or 40%, or 50%, or 60%, or 70%, or 100%.

In some embodiments, the present invention is directed at oral buprenorphine dosage forms which are therapeutically effective but which deter the abuse and misuse of the dosage form by recreational drug users of opioids and opioid addicts by rendering the dosage devoid of or substantially devoid of euphoria, pleasurable, drug liking or other mood alerting effects when taken episodically, intermittently, occasionally, periodically, on an as needed basis, or PRN, said dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release. In some embodiments, said dosage form reduces euphoria, pleasurable, drug liking or other mood alerting effects by more than about 1%, or 2%, or 3%, or 5%, or 7%, or 10%, or 12%, or 15%, or 18%, or 20%, or 25%, or 30%, or 35%, or 40%, or 50%, or 60%, or 70%, or 100%.

In some embodiments, the present invention is directed at oral buprenorphine dosage forms which are therapeutically effective but which deter the abuse and misuse of the dosage form by recreational drug users of opioids and opioid addicts by rendering the dosage devoid of or substantially devoid of the instant gratification from euphoria, pleasurable, drug liking or other mood alerting effects sought by recreational drug users of opioids and opioid addicts, said dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release. In some embodiments, said dosage form reduces the instant gratification from euphoria, pleasurable, drug liking or other mood alerting effects by more than about 1%, or 2%, or 3%, or 5%, or 7%, or 10%, or 12%, or 15%, or 18%, or 20%, or 25%, or 30%, or 35%, or 40%, or 50%, or 60%, or 70%, or 100%.

In some embodiments, the orally administered buprenorphine dosage forms of the invention have a reduced potential for abuse and misuse, including frequency, duration or magnitude of euphoria, sedation, drug liking, fatigue, cognitive impairment, motor impairment and CNS impairment.

In some embodiments, the orally administered buprenorphine dosage forms of the invention have a reduced street value, or a reduced frequency, duration or magnitude of sedation, drug liking fatigue, cognitive impairment, motor impairment and CNS impairment when assessed 0.5, or 1, or 1.5, or 2, or 2.5 or 3, or 4, or 5 or 6, or 7 or 8 hours after ingestion of the initial dose, or a subsequent dose by recreational drug users, drug addicts, or opioid naïve healthy subjects.

In some embodiments, the orally administered buprenorphine dosage forms of the invention have a reduced street value, or a reduced frequency, duration or magnitude of nausea, vomiting, sedation, drug liking fatigue, cognitive impairment, motor impairment, CNS impairment and other side effects, when assessed 0.5, or 1, or 1.5, or 2, or 2.5 or 3, or 4, or 5 or 6, or 7 or 8 hours after ingestion of the initial dose, or a subsequent dose by recreational drug users, drug addicts, or opioid naïve healthy subjects, when compared with the an oral immediate release solid or solution dosage form of buprenorphine, or a sublingual (or buccal or mucoretentive) dosage form, or an oral extended release dosage form of buprenorphine. In some embodiments, said oral immediate release solid or solution dosage form or said sublingual (or buccal or mucoretentive) dosage form of buprenorphine is administered at about the same dose, or at ≦ about 90%, or ≦ about 80%, or ≦ about 75%, at ≦ about 70%, or ≦ about 60%, or ≦ about 50%, or ≦ about 45%, or ≦ about 40%, or ≦ about 35%, at ≦ about 30%, or ≦ about 25%, or ≦ about 20%, or ≦ about 15% of the dose of orally administered buprenorphine of the invention.

It is an object of certain embodiments of the invention to provide dosage forms of oral buprenorphine which are self-titrating following first administration.

It is an object of certain embodiments of the invention to provide extended release; or sustained release; or controlled release; or delayed onset, rapid release; or delayed onset, extended release; or delayed onset, pulsatile release; or duodenal release; or jejunal release; or ileal release; or ileo-colonic release; or colonic release dosage forms of oral buprenorphine which are self-titrating following first administration to opioid naïve subjects.

It is an object of certain embodiments of the invention to provide dosage forms of oral buprenorphine which are self-titrating following first administration to opioid naïve subjects.

It is an object of certain embodiments of the invention to provide dosage forms of oral buprenorphine which are self-titrating following first administration to subjects who are not opioid tolerant.

It is an object of certain embodiments of the invention to provide dosage forms of oral buprenorphine which are self-titrating following first administration to subjects who are not receiving continuous or around the clock opioid therapy.

It is an object of certain embodiments of the invention to provide dosage forms of oral buprenorphine which are self-titrating following first administration to subjects with pain. In some embodiments, said minimal dose increase is required in less than 1%, 2%, 3%, 4%, 5%, 6%, 7%, 8%, 9%, 10%, 12%, 15%, 18%, 20%, 25%, 30%, 35%, or 40%, or 50% of subjects. This is in sharp contrast to other opioids, including other controlled release or extended release opioids for the treatment of chronic condition which require lower initial doses to attain an optimal balance between therapeutic outcome (e.g., pain relief) and side effects, and gradual dose increases after the subject has developed improved tolerability to the opioid. As used herein, “self-titrating” means that following first administration (or following initiation of therapy) at the recommended or approved starting dose, or the recommended or approved usual dose, the oral dosage form of the invention requires minimal or no dose increases or upward dose adjustments over the first few days, first few weeks or first few months of treatment for a chronic condition.

In some embodiments, no dose increase is required during the first month of treatment in about 90% of the subjects. In other embodiments, less than 1 in 100, or less than 1 in 70, or less than 1 in 50, or less than 1 in 40, or less than 1 in 30, or less than 1 in 20, or less than 1 in 15, or less than 1 in 12 or, less than 1 in 10, or less than 1 in 9, or less than 1 in 8, or less than 1 in 7, or less than 1 in 6, or less than 1 in 5, or less than 1 in 4 subjects requires a dose increase during the first month of treatment.

It is an object of certain embodiments of the invention to provide dosage forms of oral buprenorphine which are self-titrating and which provide effective therapeutic outcomes to the subject (e.g., pain relief and tolerability of side effects) with the first dose and at steady state.

In some embodiments, the number of dose changes required to reach adequate or optimal therapeutic outcomes (e.g., pain relief and tolerability of side effects) with oral buprenorphine dosage forms of the invention (e.g., controlled release, extended release, sustained release, modified release, delayed onset, rapid release or delayed onset, extended release, or delayed onset, pulsatile release, duodenal release, jejunal release, ileal release, ileo-colonic release, or colonic release dosage forms) is at least about 5%, 7%, 10%, 12%, 15%, 17%, 20%, 25%, 30%, 35%, 40%, 45%, or 50% less than with oral morphine, oral oxycodone, oral hydromorphone or transdermal fentanyl

In some embodiments, the present invention discloses that the dose range required to control pain with oral buprenorphine dosage forms of the invention in about 90% of subjects is less than or substantially less than the 8-fold dose range required with morphine, thereby providing several potential benefits, including more efficient titration process (adjusting the subject's dosage to provide acceptable pain relief without unacceptable side effects), more therapeutically and cost efficient control of symptoms, faster control of symptoms, reduced cost of goods, reduced need for dose titration, reduced need for additional visits to the clinician, reduced need for a wider range of dosage strengths and reduced pharmacy inventory. In some embodiments, the dose range of oral buprenorphine dosage forms of the invention (e.g., controlled release, extended release, sustained release, modified release, delayed onset, rapid release or delayed onset, extended release, or delayed onset, pulsatile release, duodenal release, jejunal release, ileal release, ileo-colonic release, or colonic release dosage forms) is at least about 5%, 7%, 10%, 12%, 15%, 17%, 20%, 25%, 30%, 35%, 40%, 45%, 50%, 60%, 70% or 75% less than oral morphine.

In some embodiments, the present invention discloses that the dose range required to control pain with oral buprenorphine dosage forms of the invention in about 90% of subjects is less than or substantially less than the 4-fold dose range required with oxycodone, thereby providing several potential benefits, including more efficient titration process (adjusting the subject's dosage to provide acceptable pain relief without unacceptable side effects), more therapeutically and cost efficient control of symptoms, faster control of symptoms, reduced cost of goods, reduced need for dose titration, reduced need for additional visits to the clinician, reduced need for a wider range of dosage strengths and reduced pharmacy inventory. In some embodiments, the dose range of oral buprenorphine dosage forms of the invention (e.g., controlled release, extended release, sustained release, modified release, delayed onset, rapid release or delayed onset, extended release, or delayed onset, pulsatile release, pulsatile release, duodenal release, jejunal release, ileal release, ileo-colonic release, or colonic release dosage forms) is at least about 5%, 7%, 10%, 12%, 15%, 17%, 20%, 25%, 30%, 35%, 40%, 45%, or 50% less than oral oxycodone.

In some preferred embodiments, dose range required to control pain in about 90% of subjects with oral buprenorphine dosage forms of the invention is about 1.5 fold, 2 fold, 2.5 fold, 3 fold, 3.5 fold or 4 fold, or not more than about 1.5 fold, 2 fold, 2.5 fold, 3 fold, 3.5 fold or 4 fold.

In some preferred embodiments, dose range required to control pain in about 90% of subjects with controlled release; or extended release; or sustained release; or modified release; or delayed onset, rapid release; or delayed onset, extended release; or delayed onset, pulsatile release dosage forms of oral buprenorphine is about 1.5 fold, 2 fold, 2.5 fold, 3 fold, 3.5 fold or 4 fold, or not more than about 1.5 fold, 2 fold, 2.5 fold, 3 fold, 3.5 fold or 4 fold.

In some preferred embodiments, dose range required to control pain in about 90% of subjects with duodenal release, jejunal release, ileal release, ileo-colonic release, or colonic release dosage forms of oral buprenorphine is about 1.5 fold, 2 fold, 2.5 fold, 3 fold, 3.5 fold or 4 fold, or not more than about 1.5 fold, 2 fold, 2.5 fold, 3 fold, 3.5 fold or 4 fold.

In some preferred embodiments where the dosage form is a delayed onset oral buprenorphine which releases most, substantially all or all the active drug after an initial delay, said delayed onset dosage form is limited to compositions that deliver or release drug distal to the jejunum (i.e., ileal release, ileo-colonic release or colonic release).

In some preferred embodiments, oral buprenorphine daily dose range required to control pain in about 90% of subjects is 2.5 mg to 15 mg, or 2.5 mg to 12.5 mg, or 2.5 mg to 10 mg, or 5 mg to 30 mg, or 5 mg to 25 mg, or 5 mg to 20 mg, or 5 mg to 15 mg, or 7.5 mg to 25 mg, or 7.5 mg to 20 mg, or 7.5 mg to 15 mg, or 10 mg to 50 mg, or 10 mg to 40 mg, or 10 mg to 35 mg, or 10 mg to 30 mg, or 10 mg to 25 mg, or 10 mg to 20 mg. In certain particularly preferred embodiments, the oral buprenorphine daily dose range required to control pain in about 90% of subjects is 5 mg to 20 mg or 5 mg to 15 mg, or 10 mg to 40 mg or 10 mg to 30 mg.

It should be noted that while the oral buprenorphine daily dose range required to control pain in about 90% of subjects in some embodiments of the invention is less than or substantially less than the 8-fold dose range seen with morphine which is the prototype opioid agonist, some patients will require doses that exceed or far exceed the dose range required to control pain in a substantial majority of subjects (e.g., 5, 10, 15 or 20 fold higher than the dose range required to control pain in about 90% of subjects), due to a variety of pharmacokinetic and pharmacodynamic factors known in the art.

In some preferred embodiments, the dosage form provides a method for reducing the range in daily dosages required to control pain in substantially all human patients, comprising administering a modified release oral buprenorphine comprising from about 10 mg to about 40 mg of buprenorphine, which provides a mean a systemic exposure of buprenorphine as assessed by the mean buprenorphine area under the plasma concentration time curve over two consecutive dosing intervals (AUC₀₋₂₄) from about 3200 pg·hr/mL to about 190,000 pg·hr/mL, and a mean maximum plasma concentration of buprenorphine from the first of the two consecutive doses from about 60 pg/mL to about 1800 pg/ml after repeated oral administration every 12 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 10 mg to about 40 mg of buprenorphine, a pharmaceutically acceptable salt thereof, or a mixture thereof, which provides a mean maximum plasma concentration of buprenorphine from about 60 pg/mL to about 1800 pg/ml from a mean of about 2.5 hours to about 5 hours, and a mean minimum plasma concentration of buprenorphine of about 30 pg/mL to about 900 pg/ml measured from a mean of about 8 to about 15 hours after repeated oral administration every 12 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 10 mg to about 40 mg of buprenorphine, a pharmaceutically acceptable salt thereof, or a mixture thereof, which provides a mean a systemic exposure of buprenorphine as assessed by the mean buprenorphine area under the plasma concentration time curve over two consecutive dosing intervals (AUC₀₋₂₄) from about 3200 pg·hr/mL to about 190,000 pg·hr/mL, and a mean maximum plasma concentration of buprenorphine from the first of the two consecutive doses from about 60 pg/mL to about 1800 pg/ml from a mean of about 2.5 hours to about 5 hours after repeated oral administration every 12 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 10 mg to about 40 mg of buprenorphine, a pharmaceutically acceptable salt thereof, or a mixture thereof, which provides a mean a systemic exposure of buprenorphine as assessed by the mean buprenorphine area under the plasma concentration time curve over two consecutive dosing intervals (AUC₀₋₂₄) from about 3200 pg·hr/mL to about 190,000 pg·hr/mL, a mean maximum plasma concentration of buprenorphine from the first of the two consecutive doses from about 60 pg/mL to about 1800 pg/ml from a mean of about 2.5 hours to about 5 hours, and a mean minimum plasma concentration of buprenorphine of about 30 pg/mL to about 900 pg/ml measured from a mean of about 8 to about 15 hours after repeated oral administration every 12 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 10 mg to about 40 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 3200 pg·hr/mL to about 190,000 pg·hr/mL, and a mean maximum plasma concentration of buprenorphine from about 60 pg/mL to about 1800 pg/ml after repeated oral administration every 24 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 10 mg to about 40 mg of buprenorphine, a pharmaceutically acceptable salt thereof, or a mixture thereof, which provides a mean maximum plasma concentration of buprenorphine from about 60 pg/mL to about 1800 pg/ml from a mean of about 3.5 hours to about 18 hours, and a mean minimum plasma concentration of buprenorphine of about 30 pg/mL to about 900 pg/ml from a mean of about 20 to about 28 hours after repeated oral administration every 24 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 10 mg to about 40 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 3200 pg·hr/mL to about 190,000 pg·hr/mL, and a mean maximum plasma concentration of buprenorphine from about 60 pg/mL to about 1800 pg/ml from a mean of about 3.5 hours to about 18 hours after repeated oral administration every 24 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 10 mg to about 40 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 3200 pg·hr/mL to about 190,000 pg·hr/mL, a mean maximum plasma concentration of buprenorphine from about 60 pg/mL to about 1800 pg/ml from a mean of about 3.5 hours to about 18 hours, and a mean minimum plasma concentration of buprenorphine of about 30 pg/mL to about 900 pg/ml from a mean of about 20 to about 28 hours after repeated oral administration every 24 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over two consecutive dosing intervals (AUC₀₋₂₄) from about 1600 pg·hr/mL to about 285,000 pg·hr/mL, and a mean maximum plasma concentration of buprenorphine from the first of the two consecutive doses from about 30 pg/mL to about 2700 pg/ml after repeated oral administration every 12 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 5 mg to about 60 mg of buprenorphine, a pharmaceutically acceptable salt thereof, or a mixture thereof, which provides a mean maximum plasma concentration of buprenorphine from about 30 pg/mL to about 2700 pg/ml from a mean of about 2.5 hours to about 5 hours, and a mean minimum plasma concentration of buprenorphine of about 30 pg/mL to about 900 pg/ml measured from a mean of about 8 to about 15 hours after repeated oral administration every 12 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over two consecutive dosing intervals (AUC₀₋₂₄) from about 1600 pg·hr/mL to about 285,000 pg·hr/mL, and a mean maximum plasma concentration of buprenorphine from the first of the two consecutive doses from about 30 pg/mL to about 2700 pg/ml from a mean of about 2.5 hours to about 5 hours after repeated oral administration every 12 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over two consecutive dosing intervals (AUC₀₋₂₄) from about 1600 pg·hr/mL to about 285,000 pg·hr/mL, a mean maximum plasma concentration of buprenorphine from the first of the two consecutive doses from about 30 pg/mL to about 2700 pg/ml from a mean of about 2.5 hours to about 5 hours, and a mean minimum plasma concentration of buprenorphine of about 30 pg/mL to about 900 pg/ml measured from a mean of about 8 to about 15 hours after repeated oral administration every 12 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 1600 pg·hr/mL to about 285,000 pg·hr/mL, and a mean maximum plasma concentration of buprenorphine from about 30 pg/mL to about 2700 pg/ml after repeated oral administration every 24 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 5 mg to about 60 mg of buprenorphine, a pharmaceutically acceptable salt thereof, or a mixture thereof, which provides a mean maximum plasma concentration of buprenorphine from about 30 pg/mL to about 2700 pg/ml from a mean of about 3.5 hours to about 18 hours, and a mean minimum plasma concentration of buprenorphine of about 30 pg/mL to about 900 pg/ml from a mean of about 20 to about 28 hours after repeated oral administration every 24 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 1600 pg·hr/mL to about 285,000 pg·hr/mL, and a mean maximum plasma concentration of buprenorphine from about 30 pg/mL to about 2700 pg/ml from a mean of about 3.5 hours to about 18 hours after repeated oral administration every 24 hours to steady-state conditions, said dosage form providing extended release or delayed onset, extended release.

In some preferred embodiments, the dosage form provides a method for reducing the range in daily dosages required to control pain in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 1600 pg·hr/mL to about 285,000 pg·hr/mL, a mean maximum plasma concentration of buprenorphine from about 30 pg/mL to about 2700 pg/ml from a mean of about 3.5 hours to about 18 hours, and a mean minimum plasma concentration of buprenorphine of about 30 pg/mL to about 900 pg/ml from a mean of about 20 to about 28 hours after repeated oral administration every 24 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 10 mg to about 40 mg of buprenorphine, which provides a mean a systemic exposure of buprenorphine as assessed by the mean buprenorphine area under the plasma concentration time curve over two consecutive dosing intervals (AUC₀₋₂₄) from about 3200 pg·hr/mL to about 190,000 pg·hr/mL, and a mean maximum plasma concentration of buprenorphine from the first of the two consecutive doses from about 60 pg/mL to about 1800 pg/ml after repeated oral administration every 12 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 10 mg to about 40 mg of buprenorphine, a pharmaceutically acceptable salt thereof, or a mixture thereof, which provides a mean maximum plasma concentration of buprenorphine from about 60 pg/mL to about 1800 pg/ml from a mean of about 2.5 hours to about 5 hours, and a mean minimum plasma concentration of buprenorphine of about 30 pg/mL to about 900 pg/ml measured from a mean of about 8 to about 15 hours after repeated oral administration every 12 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 10 mg to about 40 mg of buprenorphine, a pharmaceutically acceptable salt thereof, or a mixture thereof, which provides a mean a systemic exposure of buprenorphine as assessed by the mean buprenorphine area under the plasma concentration time curve over two consecutive dosing intervals (AUC₀₋₂₄) from about 3200 pg·hr/mL to about 190,000 pg·hr/mL, and a mean maximum plasma concentration of buprenorphine from the first of the two consecutive doses from about 60 pg/mL to about 1800 pg/ml from a mean of about 2.5 hours to about 5 hours after repeated oral administration every 12 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 10 mg to about 40 mg of buprenorphine, a pharmaceutically acceptable salt thereof, or a mixture thereof, which provides a mean a systemic exposure of buprenorphine as assessed by the mean buprenorphine area under the plasma concentration time curve over two consecutive dosing intervals (AUC₀₋₂₄) from about 3200 pg·hr/mL to about 190,000 pg·hr/mL, a mean maximum plasma concentration of buprenorphine from the first of the two consecutive doses from about 60 pg/mL to about 1800 pg/ml from a mean of about 2.5 hours to about 5 hours, and a mean minimum plasma concentration of buprenorphine of about 30 pg/mL to about 900 pg/ml measured from a mean of about 8 to about 15 hours after repeated oral administration every 12 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 10 mg to about 40 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 3200 pg·hr/mL to about 190,000 pg·hr/mL, and a mean maximum plasma concentration of buprenorphine from about 60 pg/mL to about 1800 pg/ml after repeated oral administration every 24 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 10 mg to about 40 mg of buprenorphine, a pharmaceutically acceptable salt thereof, or a mixture thereof, which provides a mean maximum plasma concentration of buprenorphine from about 60 pg/mL to about 1800 pg/ml from a mean of about 3.5 hours to about 18 hours, and a mean minimum plasma concentration of buprenorphine of about 30 pg/mL to about 900 pg/ml from a mean of about 20 to about 28 hours after repeated oral administration every 24 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 10 mg to about 40 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 3200 pg·hr/mL to about 190,000 pg·hr/mL, and a mean maximum plasma concentration of buprenorphine from about 60 pg/mL to about 1800 pg/ml from a mean of about 3.5 hours to about 18 hours after repeated oral administration every 24 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 10 mg to about 40 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 3200 pg·hr/mL to about 190,000 pg·hr/mL, a mean maximum plasma concentration of buprenorphine from about 60 pg/mL to about 1800 pg/ml from a mean of about 3.5 hours to about 18 hours, and a mean minimum plasma concentration of buprenorphine of about 30 pg/mL to about 900 pg/ml from a mean of about 20 to about 28 hours after repeated oral administration every 24 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over two consecutive dosing intervals (AUC₀₋₂₄) from about 1600 pg·hr/mL to about 285,000 pg·hr/mL, and a mean maximum plasma concentration of buprenorphine from the first of the two consecutive doses from about 30 pg/mL to about 2700 pg/ml after repeated oral administration every 12 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 5 mg to about 60 mg of buprenorphine, a pharmaceutically acceptable salt thereof, or a mixture thereof, which provides a mean maximum plasma concentration of buprenorphine from about 30 pg/mL to about 2700 pg/ml from a mean of about 2.5 hours to about 5 hours, and a mean minimum plasma concentration of buprenorphine of about 30 pg/mL to about 900 pg/ml measured from a mean of about 8 to about 15 hours after repeated oral administration every 12 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over two consecutive dosing intervals (AUC₀₋₂₄) from about 1600 pg·hr/mL to about 285,000 pg·hr/mL, and a mean maximum plasma concentration of buprenorphine from the first of the two consecutive doses from about 30 pg/mL to about 2700 pg/ml from a mean of about 2.5 hours to about 5 hours after repeated oral administration every 12 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over two consecutive dosing intervals (AUC₀₋₂₄) from about 1600 pg·hr/mL to about 285,000 pg·hr/mL, a mean maximum plasma concentration of buprenorphine from the first of the two consecutive doses from about 30 pg/mL to about 2700 pg/ml from a mean of about 2.5 hours to about 5 hours, and a mean minimum plasma concentration of buprenorphine of about 30 pg/mL to about 900 pg/ml measured from a mean of about 8 to about 15 hours after repeated oral administration every 12 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 1600 pg·hr/mL to about 285,000 pg·hr/mL, and a mean maximum plasma concentration of buprenorphine from about 30 pg/mL to about 2700 pg/ml after repeated oral administration every 24 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 5 mg to about 60 mg of buprenorphine, a pharmaceutically acceptable salt thereof, or a mixture thereof, which provides a mean maximum plasma concentration of buprenorphine from about 30 pg/mL to about 2700 pg/ml from a mean of about 3.5 hours to about 18 hours, and a mean minimum plasma concentration of buprenorphine of about 30 pg/mL to about 900 pg/ml from a mean of about 20 to about 28 hours after repeated oral administration every 24 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 1600 pg·hr/mL to about 285,000 pg·hr/mL, and a mean maximum plasma concentration of buprenorphine from about 30 pg/mL to about 2700 pg/ml from a mean of about 3.5 hours to about 18 hours after repeated oral administration every 24 hours to steady-state conditions, said dosage form providing extended release or delayed onset, extended release.

In some preferred embodiments, the dosage form provides a method for reducing the number of dose adjustments or dose titrations required to control pain over the first month of treatment in substantially all human patients, comprising administering a modified release oral buprenorphine comprising from about 5 mg to about 60 mg of buprenorphine, a pharmaceutically acceptable salt thereof, or a mixture thereof, which provides a mean a systemic exposure of buprenorphine as assessed by the mean buprenorphine area under the plasma concentration time curve over a single dosing interval (AUC₀₋₂₄) from about 1600 pg·hr/mL to about 285,000 pg·hr/mL, a mean maximum plasma concentration of buprenorphine from about 30 pg/mL to about 2700 pg/ml from a mean of about 3.5 hours to about 18 hours, and a mean minimum plasma concentration of buprenorphine of about 30 pg/mL to about 900 pg/ml from a mean of about 20 to about 28 hours after repeated oral administration every 24 hours to steady-state conditions, said dosage form providing extended release or delayed onset, extended release.

In some embodiments, by increasing the desired therapeutic effect, a smaller amount of buprenorphine is required to provide a given therapeutic effect. In addition to reducing the cost of goods, the improved therapeutic effect will reduce the street supply of the drug for misuse, abuse and tampering (e.g., use of the oral dosage form by the intravenous, inhalational, intranasal after tampering the dosage form). Underscoring its concerns about drug abuse, recently, the U.S. Food and Drug Administration made a precedential decision rejecting the approval of a bioequivalent (“A/B generic”) patch of transdermal fentanyl, solely of the basis of the amount of fentanyl in the patch, rather than the amount of fentanyl absorbed into systemic circulation compared to the innovator (Duragesic®).

A major challenge with the administration of more than one drug concurrently or in close proximity to each other is the occurrence of increased adverse effects from the combined and simultaneous high plasma concentrations of more than one drug producing the same or similar side effects through additive, super-additive or synergistic effects. For example, buprenorphine may be used in conjunction with other drugs for the treatment of the same or different condition or side effect. Such other drugs can produce similar or the same side effects as buprenorphine, including sedation, nausea, vomiting, and fatigue. Unfortunately, in many cases, using an alternative concomitant drug is not a practical option. In some embodiments, the present invention provides oral pharmaceutical compositions and methods to reduce the frequency, duration or magnitude of side effects from such concurrent, allowing for the advantageous use of drugs with similar or the same side effects as buprenorphine.

Buprenorphine is widely believed by ineffective and inappropriate to give orally, resulting in the need to: (i) administer the opioid by the parenteral route, with all of its sterility, cost, route accessibility to patients, and technical challenges; (ii) administer the drug by the transdermal route, with all of its cost and technical challenges; (iii) administer a sublingual dosage form with its lack of dose proportional bioavailability, limited dose range and higher potential for tampering and abuse.

In some embodiments of the present invention the oral dosage from delivers most, substantially all or all of the buprenorphine into the lower segments of the gastrointestinal tract (e.g., delayed onset, rapid release; or delayed onset, extended release; or delayed onset, pulsatile release; or duodenal release; or jejunal release; or ileal release; or ileo-colonic release; or colonic release) and the dosage form is best suited under certain conditions for the treatment of signs and symptoms which do not require a rapid onset of effect from “one of”, intermittent or episodic use of the dosage form or which are not administered as a single dose, or an intermittent dose or for episodic use. For example, if an individual has a new onset “muscle tension headache or a new onset migraine, in some embodiments of the invention where the dosage form of the present invention delivers most, substantially all or all of the buprenorphine into the lower segments of the gastrointestinal tract, it would usually be impractical to use the dosage form as a meaningful pharmacologic response may take unacceptably long to manifest itself. In some embodiments, this delay in onset of effect with single-doses, intermittent doses or episodic use contributes to providing a buprenorphine dosage form with lower abuse potential.

In some embodiments, the present invention makes it medically and pharmacologically feasible for the first time to administer buprenorphine by the oral route.

Without being bound by theory, at first glance, a major potential concern with some embodiments of the present invention (e.g., delayed onset, rapid release; or delayed onset, extended release; or delayed onset, pulsatile release; or duodenal release; or jejunal release; or ileal release; or ileo-colonic release; or colonic release) would be whether such compositions would provide continuous or around the clock plasma concentration and therapeutic effect, in view of the delayed release of drug following ingestion. In some embodiments, the invention deals with this issue in the following way: (i) for oral buprenorphine formulations whose release is delayed but subsequently immediate upon arriving at the target site in the lower segments of the gastrointestinal tract, the current dose would provide a therapeutic effect until the next dose of the invention reaches the target site; (ii) for oral buprenorphine formulations whose release is delayed but when initiated it is subsequently further retarded at the target site(s) in the lower segments of the gastrointestinal tract (e.g., controlled release, extended release or sustained release), the current dose would provide a therapeutic effect until the next dose of the invention reaches the target site. Thus, in some embodiments of the invention, the only potential “therapeutic gap” of relates to the delay with therapeutic effect with the initial or first dose of the drug. This may be dealt with by using the dosage form of the drug in conventional or immediate release form on a one time basis, or by use of an alternate drug with the same or similar therapeutic effect or by limiting such dosage forms to individuals requiring repeated or multiple dose therapy, time contingent therapy, treatment lasting more than a few days, or chronic therapy, or by excluding individuals requiring single doses, rapid onset of effect with the initial dose, intermittent dosing, episodic dosing. This potential “therapeutic gap” also provides for some of the abuse deterrent properties of the invention.

In some embodiments, the dosage form of the invention is a modified release oral formulation which, after an initial in vivo lag period lasting at least about 1, 1.5, 2, 2.5, 3, 3.5, 4, 4.5, 5, 5.5, 6, 6.5, 7, 7.5, 8, 8.5, 9, 9.5, 10, 10.5, 11, 11.5 or 12 hours during which there is little or no release of buprenorphine, rapidly releases (e.g., over a period of less than about 5, 10, 15, 30, or 45 minutes, or 1, 1.5, 2, 2.5, 3, 3.5, 4, 4.5, 5, 5.5 or 6 hours) most, substantially all or all of the buprenorphine in vivo.

In some embodiments, the dosage form of the invention is a modified release oral formulation which, after an initial in vivo lag period lasting at least about 1, 1.5, 2, 2.5, 3, 3.5, 4, 4.5, 5, 5.5, 6, 6.5, 7, 7.5, 8, 8.5, 9, 9.5, 10, 10.5, 11, 11.5 or 12 hours during which there is little or no release of buprenorphine, gradually or slowly releases (e.g., over a period of about 2, 3, 4, 5, 6, 7, 8, 10, 12, 14, 16, 18, 20, 21, 22, 23, 24, 28, 30, 36 or 40 hours) most, substantially all or all of the buprenorphine in vivo, said product characteristics observed after administration of some, most or substantially all or all doses.

In some embodiments, the dosage form of the invention is a modified release oral formulation which, after ingestion is characterized by little or no release of the buprenorphine in the stomach, said dosage form releases most, substantially all or all of the buprenorphine from the dosage form in the duodenum, jejunum, ileum and/or colon, over a period of less than about 5, 10, 15, 30, or 45 minutes, or 1, 1.5, 2, 2.5, 3, 3.5, 4, 4.5, 5, 5.5 or 6 hours.

In some embodiments, the dosage form of the invention is a modified release oral formulation which, after ingestion is characterized by little or no release of the buprenorphine in the stomach or duodenum, said dosage form releases most, substantially all or all of the buprenorphine from the dosage form upon arrival in the jejunum, ileum and/or colon, over a period of less than about 5, 10, 15, 30, or 45 minutes, or 1, 1.5, 2, 2.5, 3, 3.5, 4, 4.5, 5, 5.5 or 6 hours.

In some embodiments, the dosage form of the invention is a modified release oral formulation which, after ingestion is characterized by little or no release of the buprenorphine in the stomach, duodenum or jejunum, said dosage form releases most, substantially all or all of the buprenorphine from the dosage form upon arrival in the ileum and/or colon, over a period of less than about 5, 10, 15, 30, or 45 minutes, or 1, 1.5, 2, 2.5, 3, 3.5, 4, 4.5, 5, 5.5 or 6 hours.

In some embodiments, the dosage form of the invention is a modified release oral formulation which, after ingestion is characterized by little or no release of the buprenorphine in the stomach, duodenum, jejunum or ileum, said dosage form releases most, substantially all or all of the buprenorphine from the dosage form upon arrival in the colon, over a period of less than about 5, 10, 15, 30, or 45 minutes, or 1, 1.5, 2, 2.5, 3, 3.5, 4, 4.5, 5, 5.5 or 6 hours.

In some embodiments, the dosage form of the invention is a modified release oral formulation which, after ingestion is characterized by little or no release of the buprenorphine in the stomach, said dosage form releases most, substantially all or all of the buprenorphine from the dosage form in the duodenum, jejunum, ileum and/or colon gradually over a period of not less than about 7, 8, 10, 12, 14, 16, 18, 20, 21, 22, 23, 24, 28, 30, 36 or 40 hours.

In some embodiments, the dosage form of the invention is a modified release oral formulation which, after ingestion is characterized by little or no release of the buprenorphine in the stomach or duodenum, said dosage form releases most, substantially all or all of the buprenorphine from the dosage form upon arrival in the jejunum, ileum and/or colon gradually over a period of not less than about 7, 8, 10, 12, 14, 16, 18, 20, 21, 22, 23, 24, 28, 30, 36 or 40 hours.

In some embodiments, the dosage form of the invention is a modified release oral formulation which, after ingestion is characterized by little or no release of the buprenorphine in the stomach, duodenum or jejunum, said dosage form releases most, substantially all or all of the buprenorphine from the dosage form upon arrival in the ileum and/or colon gradually over a period of not less than about 7, 8, 10, 12, 14, 16, 18, 20, 21, 22, 23, 24, 28, 30, 36 or 40 hours.

In some embodiments, the dosage form of the invention is a modified release oral formulation which, after ingestion is characterized by little or no release of the buprenorphine in the stomach, duodenum, jejunum or ileum, said dosage form releases most, substantially all or all of the buprenorphine from the dosage form upon arrival in the colon gradually over a period of not less than about 7, 8, 10, 12, 14, 16, 18, 20, 21, 22, 23, 24, 28, 30, 36 or 40 hours.

In some embodiments, the described in vitro or in vivo (including gastrointestinal tract and systemic) release characteristics, specifications and claims of the dosage forms of the invention are observed after some, or most, or substantially all, or all administered or ingested doses.

In some embodiments, the described pharmacokinetic and pharmacodynamic characteristics, specifications and claims of the dosage forms of the invention are observed after some, or most, or substantially all, or all administered or ingested doses.

In some embodiments, the described in vitro or in vivo (including gastrointestinal tract and systemic) release characteristics, specifications and claims of the dosage forms of the invention are observed after first administration.

In some embodiments, the described pharmacokinetic and pharmacodynamic characteristics, specifications and claims of the dosage forms of the invention are observed after first administration.

In some embodiments, the described in vitro or in vivo (including gastrointestinal tract and systemic) release characteristics, specifications and claims of the dosage forms of the invention are observed after repeated or multiple administration.

In some embodiments, the described pharmacokinetic and pharmacodynamic characteristics, specifications and claims of the dosage forms of the invention are observed after repeated or multiple administrations.

In some embodiments, the oral modified release buprenorphine dosage form is enteric coated, or sequestered so as to release little or no buprenorphine in the stomach, or stomach and duodenum, or stomach, duodenum and jejunum, or stomach, duodenum, jejunum and ileum, or stomach, duodenum, jejunum, ileum and ileo-cecal junction.

In some embodiments, the oral modified release buprenorphine dosage form provides an oral pharmaceutical composition and method of treating a buprenorphine responsive medical condition, said treatment (i) achieving the same therapeutic objectives with a reduced dose of buprenorphine; (ii) providing improved dose proportional extent of absorption; (iii) a more consistent clinical effect; (iv) a more consistent pharmacokinetic effect; (v) more consistent extent of oral absorption; (vi) greater efficacy; (vii) reduced frequency, duration and/or magnitude of side effects; (viii) reduced frequency, duration and/or magnitude of side effects at or for up to 0.5, 1, 2, 3, 4, 5, 6, 7 or 8 hours after the first dose; (ix) reduced frequency, duration and/or magnitude of nausea, vomiting and/or drowsiness; (x) reduced potential for drug abuse, drug diversion, drug liking, and mood altering effects; (xi) reduced potential for euphoria, drug liking, and mood altering effects; (xii) more efficient clinical management; (xiii) reduced metabolite accumulation (reduced metabolite to parent drug ratio), including in subjects with renal impairment; (xiv) reduced metabolite related side effects, e.g., neurotoxicity and myoclonus.

In some embodiments, the oral modified release buprenorphine dosage form demonstrate increased efficacy of at least about 1%, 2%, 3%, 4%, 5%, 6%, or 8%, 10%, 12%, 15%, 20%, 30%, or 40%, when compared with oral immediate release dosage forms.

In some embodiments, the oral modified release buprenorphine dosage form demonstrate reduced euphoria, drug liking, mood altering effects, nausea, vomiting, bluffed vision, fatigue and/or drowsiness of at least about 1%, 2%, 3%, 4%, 5%, 6%, or 8%, 10%, 12%, 15%, 20%, 30%, or 40%, when compared with oral immediate release dosage forms.

In some embodiments, the oral modified release buprenorphine dosage form demonstrate reduced euphoria, drug liking, mood altering effects, nausea, vomiting, bluffed vision, fatigue and/or drowsiness of at least about 1%, 2%, 3%, 4%, 5%, 6%, or 8%, 10%, 12%, 15%, 20%, 30%, or 40%, when compared with sublingual, buccal or mucoretentive dosage forms.

In some embodiments, the dosage form of the invention is an oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release.

In some embodiments, the dosage form of the invention is an oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release, said delayed release rendering said dosage form abuse resistant.

In some embodiments, the dosage form of the invention is an oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release, said dosage form abuse resistant compared with sublingual buprenorphine. In some embodiments, said modified release dose is at least about 20%, or 30%, or 40%, or 50%, or 60%, or 75%, or 100%, or 150%, or 200%, or 250%, or 300%, or 350%, or 400%, or 450%, or 500%, or 700%, or 1000% greater than said sublingual dose.

In some embodiments, the dosage form of the invention is an oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release, said dosage form abuse resistant compared with oral immediate release buprenorphine. In some embodiments, said modified release dose is at least about 20%, or 30%, or 40%, or 50%, or 60%, or 75%, or 100%, or 150%, or 200%, or 250%, or 300%, or 350%, or 400%, or 450%, or 500%, or 700%, or 1000% greater than said oral immediate release dose.

In some embodiments, the dosage form of the invention is an oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release, said dosage form abuse resistant compared with oral dosage forms of buprenorphine suitable for pulsatile release but without a delay in release of the initial pulse. In some embodiments, said modified release dose is at least about 20%, or 30%, or 40%, or 50%, or 60%, or 75%, or 100%, or 150%, or 200%, or 250%, or 300%, or 350%, or 400%, or 450%, or 500%, or 700%, or 1000% greater than said oral pulsatile dose.

In some embodiments, the dosage form of the invention is an oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release, said dosage form abuse resistant compared with oral dosage forms of buprenorphine suitable for modified release but without delayed release. In some embodiments, said modified release dose with delayed onset is at least about 10%, 20%, or 30%, or 40%, or 50%, or 60%, or 75%, or 100%, or 150%, or 200%, or 250%, or 300%, greater than said comparator dose of modified release without delayed onset.

In some preferred embodiments, the controlled release material of the oral dosage form of the invention further comprises material to render said composition resistant or substantially resistant to dissolution in the stomach, or in the duodenum, or in the jejunum, or in the ileum, or in the small intestine, or in the stomach and duodenum, or in the stomach, duodenum and jejunum, or in the stomach, duodenum, jejunum and terminal ileum, in the stomach and small intestine, or before it reaches the ileo-cecal junction, or until it crosses the ileo-cecal junction or until it reaches the colon.

In some preferred embodiments, the controlled release material of the oral dosage form of the invention further comprises an overcoat material or an embedded material to render said composition resistant or substantially resistant to dissolution in the stomach, or in the duodenum, or in the jejunum, or in the ileum, or in the small intestine, or in the stomach and duodenum, or in the stomach, duodenum and jejunum, or in the stomach, duodenum, jejunum and terminal ileum, in the stomach and small intestine, or before it reaches the ileo-cecal junction, or until it crosses the ileo-cecal junction or until it reaches the colon. In some preferred embodiments, said overcoat is additionally incorporated into or applied over immediate release dosage forms, and controlled release matrix dosage forms, including controlled porosity osmotic dosage forms, push pull osmotic dosage forms.

As used herein with respect to the buprenorphine dosage form of the invention, the term “oral”, “oral dosage form”, “oral pharmaceutical dosage form”, “oral administration”, “oral compositions” “oral pharmaceutical compositions”, “oral tablets”, “oral capsules”, “orally ingested”, “orally”, “oral route” and the like all refer to any method of administration through the mouth for rapid deposit of the dosage form into the stomach or alimentary canal. The oral dosage form of the invention is usually ingested intact, although it may be ingested un-intact or tampered (e.g., crushed) and usually with the aid of water or a beverage to hasten passage through the mouth. Specifically excluded from this invention and the forgoing definition of oral dosage forms are buprenorphine dosage forms and pharmaceutical compositions administered by the lingual, sublingual, oro-mucosal, transmucosal and buccal routes. Lingual, sublingual, oro-mucosal, transmucosal and buccal routes of administration are intended to provide absorption or substantial absorption of the drug in the oral cavity (i.e., the mouth) through rapid or slow dissolution in the oral cavity and/or through prolonged residence in the oral cavity (i.e., oral cavity residence time beyond the usual time associated with oral ingestion of drug intended to be deposited into the stomach or alimentary canal). Such formulations and their method of administration are well known in the art and may under certain conditions include lozenges, transmucosal films, buccal products, mucoretentive products, orally disintegrating tablets, fast dissolving tablets, fast dispersing tablets, fast disintegrating dosage forms, provided they are intended for absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach).

As used herein, “controlled release material”, “controlled release means”, and “material to provide controlled release” means an in vitro or in vivo release rate controlling excipient or material incorporated in the dosage form whose function or primary function is to modify release (e.g, onset of release, rate of release, duration of release) of an active drug (e.g., buprenorphine) from a dosage form or a portion (i.e., cause the dosage form to release in other than an immediate release fashion). In particularly preferred embodiments of the invention, the controlled release material functions to provide one or more of the following: (1) delay in the onset of release; (2) delay in the rate of release; (3) delay in the duration of release; (4) prolonged or extended duration of release; (5) pulsatile release; (6) delay in the onset of therapeutic effect; (7) delay in onset of side effects; (8) delay in the onset of psychic or mood altering effects; (9) reduced abuse liability; (10) prolonged or extended duration of therapeutic effect; or (11) more robust therapeutic effect; (12) a pharmacokinetic profile consistent with dosage forms which are controlled release, extended release, sustained release, delayed onset, rapid release or delayed onset, extended release, or delayed onset, pulsatile release, or modified release, or slow release or prolonged release; (13) a pharmacodynamic profile consistent with dosage forms which are controlled release, extended release, sustained release, delayed onset, rapid release or delayed onset, extended release, or delayed onset, pulsatile release, or modified release, or slow release or prolonged release (14) efficacy or improved efficacy by the oral route; (15) improved safety by the oral route; (16) improved efficiency of the dosage form; (17) improved dose proportional extent of absorption; (18) reduced variability in absorption; (19) provide pulsatile release of the active drug; (20) provide delayed onset, rapid release of the dosage form; (21) provide delayed onset, extended release of the dosage form; (22) improved efficacy through delayed onset, rapid release or delayed onset, extended release compared with immediate release oral dosage forms; and (23) reduced potential for misuse, abuse, addiction and drug diversion.

In some preferred nonlimiting examples, incorporation of controlled release material into the dosage form can provide for one or more of the following: (1) delay in release (for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), where the onset of first release or first substantial release is delayed but after start of said release, most, substantially all, or all of the active drug is rapidly released or liberated from the dosage form; (2) delay in release (for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), where the onset of first release or first substantial release is delayed but after start of said release, the active drug is rapidly released or liberated from the dosage form; (3) a delay in release, where, (a) the onset of first release or first substantial release is delayed until a specified or preferred time after oral ingestion (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said release, most, substantially all, or all of the active drug is rapidly released or liberated from the dosage form (e.g., over a period of a few minutes to a few hours, e.g., in less than about 0.05, 0.1, 0.2, 0.3, 0.4, 0.5, 0.75, 1, 1.5, 2, 2.5, 3, or 3.5 hours); (4) a delay in release, where, (a) the onset of first release or first substantial release is delayed until a specified or preferred time after oral ingestion (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said release, the active drug is rapidly released or liberated from the dosage form (e.g., over a period of a few minutes to a few hours, e.g., in less than about 0.05, 0.1, 0.2, 0.3, 0.4, 0.5, 0.75, 1, 1.5, 2, 2.5, 3, or 3.5 hours); (5) delay in release, where the onset of first release or first substantial release is delayed (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours) but after start of said release, most, substantially all, or all of the active drug is slowly released or liberated from the dosage form; (6) delay in release, where the onset of first release or first substantial release is delayed (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours) but after start of said release, the active drug is slowly released or liberated from the dosage form; (7) delayed onset, extended release, where, (a) the onset of first release or first substantial release is delayed until a specified or preferred time after oral ingestion (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said release, most, substantially all, or all of the active drug is slowly released or liberated from the dosage form (e.g, prolonged release, or extended release, or sustained release, or slow release, or release over a period of more than about 4, or 4.5, or 5, or 5.5, or 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (8) delayed onset, rapid release, where, (a) the onset of first release or first substantial release is delayed until a specified or preferred time after oral ingestion (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said release, the active drug is rapidly released or liberated from the dosage form over a period of less than about 0.125, 0.25, 0.5, 0.75, 1, 1.5, 2, 2.5, or 3, or 3.5; (8b) “delayed onset, pulsatile release”, where, (a) the onset of first release or first substantial release is delayed until a specified or preferred time after oral ingestion (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said release, rapidly release the active drug from the dosage form in “pulses” at their desired time intervals (e.g, about every 2, 3, 4, 5, 6, 8 or 12 hours), each pulse releasing a portion of the active drug rapidly (e.g., over about 0.125, 0.25, 0.5, 0.75, 1, 1.5, 2, 2.5, 3, 3.5 hours from the desired time); (9) rapid onset of first release or first substantial release, with continued slow release or liberation of the active drug from the dosage form; (10) rapid onset of first release or first substantial release (e.g., in less than about 0.05, 0.1, 0.2, 0.3, 0.4, 0.5, 0.75, 1, 1.5, 2, 2.5, or 3 hours, preferably less than 0.5, 1, 1.5, or 2 3 hours), with continued slow release or liberation of the active drug from the dosage form over a period of up to about 4, or 4.5, or 5, or 5.5, or 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (10a) slow onset of first release or first substantial release, with continued slow release or liberation of the active drug from the dosage form; (11) slow onset of first release or first substantial release (e.g., in less than about 0.05, 0.1, 0.2, 0.3, 0.4, 0.5, 0.75, 1, 1.5, 2, 2.5, or 3 hours, preferably less than 0.5, 1, 1.5, or 2 3 hours), with continued slow release or liberation of the active drug from the dosage form over a period of up to about 4, or 4.5, or 5, or 5.5, or 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (12) no release or substantially no release in the stomach, followed by first release or first substantial release of the active drug from the dosage form in the duodenum or distal to the duodenum, where, after start of said first release, the active drug is rapidly released or liberated from the dosage form; (13) no release or substantially no release in the stomach, followed by first release or first substantial release of the active drug from the dosage form in the duodenum or distal to the duodenum, where, after start of said first release, the active drug is rapidly released or liberated from the dosage form (e.g., over a period of a few minutes to a few hours, e.g., in less than about 0.05, 0.1, 0.2, 0.3, 0.4, 0.5, 0.75, 1, 1.5, 2, 2.5, 3, or 3.5 hours); (14) no release or substantially no release in the stomach, followed by first release or first substantial release of the active drug from the dosage form in the duodenum or distal to the duodenum, where, after start of said first release, the active drug is slowly released or liberated from the dosage form; (15) no release or substantially no release in the stomach, followed by first release or first substantial release of the active drug from the dosage form in the duodenum or distal to the duodenum, where, after start of said first release, the active drug is slowly released or liberated from the dosage form over a period of up to about 4, or 4.5, or 5, or 5.5, or 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (16) no release or substantially no release in the duodenum or proximal to the duodenum, followed by first release or first substantial release of the active drug from the dosage form in the jejunum or distal to the jejunum, where, after start of said first release, the active drug is rapidly released or liberated from the dosage form; (17) no release or substantially no release in the duodenum or proximal to the duodenum, followed by first release or first substantial release of the active drug from the dosage form in the jejunum or distal to the jejunum, where, after start of said first release, the active drug is rapidly released or liberated from the dosage form (e.g., over a period of a few minutes to a few hours, e.g., in less than about 0.05, 0.1, 0.2, 0.3, 0.4, 0.5, 0.75, 1, 1.5, 2, 2.5, 3, or 3.5 hours); (18) no release or substantially no release in the duodenum or proximal to the duodenum, followed by first release or first substantial release of the active drug from the dosage form in the jejunum or distal to the jejunum, where, after start of said first release, the active drug is slowly released or liberated from the dosage form; (19) no release or substantially no release in the duodenum or proximal to the duodenum, followed by first release or first substantial release of the active drug from the dosage form in the jejunum or distal to the jejunum, where, after start of said first release, the active drug is slowly released or liberated from the dosage form over a period of up to about 4, or 4.5, or 5, or 5.5, or 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (20) no release or substantially no release in the jejunum or proximal to the jejunum, followed by first release or first substantial release of the active drug from the dosage form in the ileum or distal to the ileum, where, after start of said first release, the active drug is rapidly released or liberated from the dosage form; (21) no release or substantially no release in the jejunum or proximal to the jejunum, followed by first release or first substantial release of the active drug from the dosage form in the ileum or distal to the ileum, where, after start of said first release, the active drug is rapidly released or liberated from the dosage form (e.g., over a period of a few minutes to a few hours, e.g., in less than about 0.05, 0.1, 0.2, 0.3, 0.4, 0.5, 0.75, 1, 1.5, 2, 2.5, 3, or 3.5 hours); (22) no release or substantially no release in the jejunum or proximal to the jejunum, followed by first release or first substantial release of the active drug from the dosage form in the ileum or distal to the ileum, where, after start of said first release, the active drug is slowly released or liberated from the dosage form; (23) no release or substantially no release in the jejunum or proximal to the jejunum, followed by first release or first substantial release of the active drug from the dosage form in the ileum or distal to the ileum, where, after start of said first release, the active drug is slowly released or liberated from the dosage form over a period of up to about 4, or 4.5, or 5, or 5.5, or 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (24) no release or substantially no release in the jejunum or proximal to the jejunum, followed by first release or first substantial release of the active drug from the dosage form in the ileo-colonic region, where, after start of said first release, the active drug is rapidly released or liberated from the dosage form; (25) no release or substantially no release in the jejunum or proximal to the jejunum, followed by first release or first substantial release of the active drug from the dosage form in the ileo-colonic region, where, after start of said first release, the active drug is rapidly released or liberated from the dosage form (e.g., over a period of a few minutes to a few hours, e.g., in less than about 0.05, 0.1, 0.2, 0.3, 0.4, 0.5, 0.75, 1, 1.5, 2, 2.5, 3, or 3.5 hours); (26) no release or substantially no release in the jejunum or proximal to the jejunum, followed by first release or first substantial release of the active drug from the dosage form in the ileo-colonic region, where, after start of said first release, the active drug is slowly released or liberated from the dosage form; (27) no release or substantially no release in the jejunum or proximal to the jejunum, followed by first release or first substantial release of the active drug from the dosage form in the ileo-colonic region, where, after start of said first release, the active drug is slowly released or liberated from the dosage form over a period of up to about 4, or 4.5, or 5, or 5.5, or 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (28) no release or substantially no release in the ileum or proximal to the ileum, followed by first release or first substantial release of the active drug from the dosage form in the colon, where, after start of said first release, the active drug is rapidly released or liberated from the dosage form; (29) no release or substantially no release in the ileum or proximal to the ileum, followed by first release or first substantial release of the active drug from the dosage form in the colon, where, after start of said first release, the active drug is rapidly released or liberated from the dosage form (e.g., over a period of a few minutes to a few hours, e.g., in less than about 0.05, 0.1, 0.2, 0.3, 0.4, 0.5, 0.75, 1, 1.5, 2, 2.5, 3, or 3.5 hours); (30) no release or substantially no release in the ileum or proximal to the ileum, followed by first release or first substantial release of the active drug from the dosage form in the colon, where, after start of said first release, the active drug is slowly released or liberated from the dosage form; (31) no release or substantially no release in the ileum or proximal to the ileum, followed by first release or first substantial release of the active drug from the dosage form in the colon, where, after start of said first release, the active drug is slowly released or liberated from the dosage form over a period of up to about 4, or 4.5, or 5, or 5.5, or 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (32) no release or substantially no release proximal to the ileo-cecal junction, followed by first release or first substantial release of the active drug from the dosage form in the colon, where, after start of said first release, the active drug is rapidly released or liberated from the dosage form; (33) no release or substantially no release proximal to the ileo-cecal junction, followed by first release or first substantial release of the active drug from the dosage form in the colon, where, after start of said first release, the active drug is rapidly released or liberated from the dosage form (e.g., over a period of a few minutes to a few hours, e.g., in less than about 0.05, 0.1, 0.2, 0.3, 0.4, 0.5, 0.75, 1, 1.5, 2, 2.5, 3, or 3.5 hours); (34) no release or substantially no release proximal to the ileo-cecal junction, followed by first release or first substantial release of the active drug from the dosage form in the colon, where, after start of said first release, the active drug is slowly released or liberated from the dosage form; (35) no release or substantially no release proximal to the ileo-cecal junction, followed by first release or first substantial release of the active drug from the dosage form in the colon, where, after start of said first release, the active drug is slowly released or liberated from the dosage form over a period of up to about 4, or 4.5, or 5, or 5.5, or 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (36) delay in release, where the onset of first release or first substantial release is delayed but after start of said release, most, substantially all, or all of the active drug is released or liberated from the dosage form in more than one pulse or burst (pulsatile release), each pulse separated from another pulse for an interval of minutes to hours; (37) delay in release (for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), where the onset of first release or first substantial release is delayed but after start of said release, most, substantially all, or all of the active drug is released or liberated from the dosage form in more than one pulse or burst (pulsatile release), each pulse separated from another pulse for an interval of minutes to hours (e.g., up to about 0.1, 0.2, 0.5, 1, 1.5, 2, 2.5, 3, 3.5, 4, 4.5, 5, 5.5, 6, 6.5, 7, 7.5, 8, 8.5, 9, 9.5, 10, 11 or 12 hours); (38) a delay in release, where, (a) the onset of first release or first substantial release is delayed until a specified or preferred time after oral ingestion, or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said release, most, substantially all, or all of the active drug is released or liberated from the dosage form in more than one pulse or burst (pulsatile release), each pulse separated from another pulse for an interval of minutes to hours; (39) a delay in release, where, (a) the onset of first release or first substantial release is delayed until a specified or preferred time after oral ingestion (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said release, most, substantially all, or all of the active drug is released or liberated from the dosage form in more than one pulse or burst (pulsatile release), each pulse separated from another pulse for an interval of minutes to hours (e.g., up to about 0.1, 0.2, 0.5, 1, 1.5, 2, 2.5, 3, 3.5, 4, 4.5, 5, 5.5, 6, 6.5, 7, 7.5, 8, 8.5, 9, 9.5, 10, 11 or 12 hours); (40) rapid onset of first release or first substantial release, with continued release or liberation of the active drug from the dosage form in more than one pulse or burst (pulsatile release), each pulse separated from another pulse for an interval of minutes to hours (e.g., up to about 0.1, 0.2, 0.5, 1, 1.5, 2, 2.5, 3, 3.5, 4, 4.5, 5, 5.5, 6, 6.5, 7, 7.5, 8, 8.5, 9, 9.5, 10, 11 or 12 hours); (41) delay in onset of therapeutic effect, where the onset of therapeutic effect or substantial therapeutic effect is delayed after first ingestion of the oral dosage form, but after start of said effect, most, substantially all, or all of the effect realized over a period of minutes to hours; (42) delay in onset of therapeutic effect, where the onset of therapeutic effect or substantial therapeutic effect is delayed after first ingestion of the oral dosage form, but after start of said effect, most, substantially all, or all of the effect is realized over a period of minutes to hours (e.g., in about 0.05, 0.1, 0.2, 0.3, 0.4, 0.5, 0.75, 1, 1.5, 2, 2.5, 3, or 3.5 hours; 4; 4.5, 5, 5.5 or 6 hours); (43) delay in onset of therapeutic effect (for up to about 0.1, 0.25, 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), where the onset of therapeutic effect or substantial therapeutic effect is delayed after the first ingestion of the oral dosage form, but after start of said effect, most, substantially all, or all of the effect realized in less than about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours; (44) a delay in onset of therapeutic effect, where (a) the onset of therapeutic effect or substantial therapeutic effect is delayed after first ingestion of the oral dosage form release is delayed until a specified or preferred time after oral ingestion, or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said effect, most, substantially all, or all of the effect is realized over a period of minutes to hours; (45) a delay in onset of therapeutic effect, where (a) the onset of therapeutic effect or substantial therapeutic effect is delayed after first ingestion of the oral dosage form release is delayed until a specified or preferred time after oral ingestion (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said effect, most, substantially all, or all of the effect is realized over a period of minutes to hours (e.g., in about 0.05, 0.1, 0.2, 0.3, 0.4, 0.5, 0.75, 1, 1.5, 2, 2.5, 3, or 3.5 hours; 4; 4.5, 5, 5.5 or 6 hours); (46) delay in onset of therapeutic effect, where the onset of therapeutic effect or substantial therapeutic effect is delayed after first ingestion of the oral dosage form but after start of said effect, most, substantially all, or all of the effect is realized over a prolonged or extended period of time; (47) delay in onset of therapeutic effect, where the onset of therapeutic effect or substantial therapeutic effect is delayed after first ingestion of the oral dosage form (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours) but after start of said effect, most, substantially all, or all of the effect is realized over a period of about 4, or 4.5, or 5, or 5.5, or 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (48) delay in onset of therapeutic effect, where the onset of therapeutic effect or substantial therapeutic effect is delayed after first ingestion of the oral dosage form (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours) but after start of said effect, the dosage form provides a therapeutic effect for a period of not less than about 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (49) delayed onset of therapeutic effect, where, (a where the onset of therapeutic effect or substantial therapeutic effect is delayed after first ingestion of the oral dosage form until a specified or preferred time after oral ingestion (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said effect, most, substantially all, or all of the effect is realized over a prolonged or extended period of time (e.g, over a period of more than about 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (50) delayed onset of therapeutic effect, where the onset of therapeutic effect or substantial therapeutic effect is delayed after first ingestion of the oral dosage form until a specified or preferred time after oral ingestion (e.g., for up to about 0.5, 1, 1.5, 2, 2.5, 3, 3.5, 4, 4.5, 5, 5.5, 6, 6.5, 7, 7.5, 8, 8.5, 9, 9.5, 10, 11 or 12 hours), or until the dosage form reaches a specified or preferred anatomic location in the GI lumen, or until the dosage form is in contact or prolonged contact with a particular in vitro or in vivo (GI) pH, or until the dosage form is in contact or prolonged contact with a preferred or specified in vitro or in vivo (GI) environment (such pH, GI luminal osmotic pressure, dosage form osmotic pressure, hydration, microbial environment, level of GI peristalsis or agitation), and (b) after the start of said effect, the dosage form provides a therapeutic effect for a period of not less than about 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (51) rapid onset of therapeutic effect, where the onset of therapeutic effect or substantial therapeutic effect is rapid after first ingestion of the oral dosage form (e.g., in less than about 0.05, 0.1, 0.2, 0.3, 0.4, 0.5, 0.75, 1, 1.5, 2, 2.5, or 3 hours, preferably less than 0.5, 1, 1.5, or 2 3 hours), with continued therapeutic effect over a period of up to about 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (52) slow onset of therapeutic effect, where the onset of therapeutic effect or substantial therapeutic effect is slow after first ingestion of the oral dosage form (e.g., more than about 2, 3, 4, 5, 6, 7, 8, 9, 10, 11 or 12 hours) but after the start of said effect, the dosage form provides a therapeutic effect for a period of not less than about 6, or 6.5, or 7, or 7.5, or 8, or 8.5 or 9, or 9.5, or 10, or 11, or 12, or 14, 16, or 18, or 20, or 22, or 24, or 26, or 28 or 30 hours; (53) no therapeutic effect or substantial therapeutic effect after first ingestion of the oral dosage form while the dosage form is in the stomach, followed by therapeutic effect or substantial therapeutic when the dosage form is in the duodenum or distal to the duodenum; (54) no therapeutic effect or substantial therapeutic effect after first ingestion of the oral dosage form while the dosage form is in the duodenum or proximal to the duodenum, followed by therapeutic effect or substantial therapeutic when the dosage form is in the jejunum or distal to the jejunum; (55) no therapeutic effect or substantial therapeutic effect after first ingestion of the oral dosage form while the dosage form is in the jejunum or proximal to the jejunum, followed by therapeutic effect or substantial therapeutic when the dosage form is in the ileum or distal to the ileum; (56) no therapeutic effect or substantial therapeutic effect after first ingestion of the oral dosage form while the dosage form is in the ileum or proximal to the ileum, followed by therapeutic effect or substantial therapeutic when the dosage form is in the colon; (58) no therapeutic effect or substantial therapeutic effect after first ingestion of the oral dosage form while the dosage form is in the jejunum or proximal to the jejunum, followed by therapeutic effect or substantial therapeutic when the dosage form is in the ileo-colonic regions; (59) slow onset of psychic or mood altering effects after first ingestion of the oral dosage form by recreational drug users (e.g., after more than about 0.25, 0.5, 0.75, 1, 1.5, 2, 3, 4, 5, 6, 7, 8, 9, 10, 11 or 12 hours); (60) slow onset of side effects after first ingestion of the oral dosage form (e.g., after more than about 0.25, 0.5, 0.75, 1, 1.5, 2, 3, 4, 5, 6, 7, 8, 9, 10, 11 or 12 hours); (61) reduced frequency or magnitude of side effects after first ingestion of the oral dosage form; (62) reduced frequency or magnitude of psychic or mood altering effects after first ingestion of the oral dosage form by recreational drug users; (63) no psychic or mood altering effects or side effects or substantial psychic or mood altering effects or side effects after first ingestion of the oral dosage form while the dosage form is in the stomach; (64) no psychic or mood altering effects or side effects or substantial psychic or mood altering effects or side effects after first ingestion of the oral dosage form while the dosage form is in the duodenum or proximal to the duodenum; (65) no psychic or mood altering effects or side effects or substantial psychic or mood altering effects or side effects after first ingestion of the oral dosage form while the dosage form is in the jejunum or proximal to the jejunum; (66) no psychic or mood altering effects or side effects or substantial psychic or mood altering effects or side effects after first ingestion of the oral dosage form while the dosage form is in the ileum or proximal to the ileum; (67) delay in time to peak plasma concentration (Tₘₐₓ) to more than about 2, 2.25, 2.5, 2.75, 3, 3.25, 3.5, 3.75, 4, 5.5, 5, 5.5, 6, 6.5, 7, 7.5, 8, 8.5, 9, 9.5, 10, 10.5, 11, 11.5, or 12 hours) after ingestion of the oral dosage form.

As used herein, the term “modified release” formulations, dosages, dosage forms, compositions, drugs, tablets, capsules or pharmaceutical compositions mean pharmaceutical preparations which release an active ingredient from a dosage form or a portion thereof in other than an immediate release fashion. Modified release pharmaceutical compositions are made by incorporating a controlled release material in the dosage form. Modified release dosage forms are sometimes designed to accomplish pharmaceutical, pharmacokinetic, pharmacodynamic, therapeutic or convenience objectives not offered by conventional dosage forms such as a solution or an immediate release dosage form. As used herein, the term “modified release” includes “delayed onset” (or “delayed release”) and “controlled release”. As used herein, “controlled release” formulations, dosages, dosage forms, compositions, drugs, tablets, capsules or pharmaceutical compositions (also referred to as “prolonged release”, “slow release”, “sustained release”, “extended release”, “retarded release”, and the like) mean pharmaceutical preparations which release an active ingredient from a dosage form or a portion thereof over an extended period of time (over a period of time greater than 4 or 6 hours, more preferably for periods of up to about 12 hours or up to about 24 hours, or longer.), either with an initial delay in release (e.g., a delay of 0.5, 1, 1.5, 2, 2.5, 3, 3.5, 4, 4.5, 5, 5.5, 6, 6.5, 7, 7.5 or 8 hours) or without an initial delay in release. As used herein, “delayed onset” and “delayed release” formulations, dosages, dosage forms, compositions, drugs, tablets, capsules or pharmaceutical compositions mean pharmaceutical preparations which release begin the first release of an active ingredient from a dosage form or a portion thereof (i) at time other than immediately following oral administration; and/or (ii) after a lag period lasting from minutes to hours (e.g., 0.5, 1, 1.5, 2, 2.5, 3, 3.5, 4, 4.5, 5, 5.5, 6, 6.5, 7, 7.5 or 8 hours); and/or (iii) upon reaching the desired or target GI anatomic location distal to the stomach (e.g., distal to the duodenum, jejunum, ileum, ileo-cecal junction or colon) or GI environment (e.g., pH at the point of release, osmotic pressure at the point of release, hydration, microbial flora). As used herein, “delayed onset, rapid release” means dosage forms which after a desired lag period post-ingestion (e.g., 0.5, 1, 1.5, 2, 2.5, 3, 3.5, 4, 4.5, 5, 5.5, 6, 6.5, 7, 7.5 or 8 hours), rapidly releases (i.e, over about 0.05, 0.1, 0.125, 0.25, 0.5, 0.75, 1, 1.5, 2, 2.5, 3, 3.5 hours, preferably over less than about 1 or 2 hours) substantially all or all the active drug from the dosage form. As used herein, “delayed onset, pulsatile release” means dosage forms which after a desired lag period post-ingestion (e.g., 0.5, 1, 1.5, 2, 2.5, 3, 3.5, 4, 4.5, 5, 5.5, 6, 6.5, 7, 7.5 or 8 hours), rapidly releases (i.e, over about 0.05, 0.1, 0.125, 0.25, 0.5, 0.75, 1, 1.5, 2, 2.5, 3, 3.5 hours, preferably over less than about 1 or 2 hours) some of the active drug from the dosage form in “pulses” at the desired time intervals (e.g, about every 0.5, 1, 1.5, 2, 3, 4, 5, 6, 8, 12 or 24 hours), each pulse releasing a portion of the active drug in the dosage form. As used herein, “delayed onset, extended release” means dosage forms which after a desired lag period post-ingestion (e.g., 0.5, 1, 1.5, 2, 2.5, 3, 3.5, 4, 4.5, 5, 5.5, 6, 6.5, 7, 7.5 or 8 hours), slowly release the active drug from the dosage form over an extended period of time (e.g., over about 4, 4.5, 5, 6, 7, 8, 9, 10, 12, 14, 16, 18, 20, 22, 24, 26, or 30 hours). As used herein, “delayed release” and “delayed onset” include dosage forms which are delayed onset, rapid release; delayed onset, pulsatile release; and delayed onset.

In some embodiments, one or more, or substantially all, or all of the embodiments and specifications showing benefit or a difference for modified release delayed onset dosage forms of oral buprenorphine (e.g., delayed onset, rapid release; delayed onset, pulsatile release; delayed onset, extended release) over immediate release oral buprenorphine, the phrase “immediate release” may be substituted with “controlled release”, “extended release” or “pulsatile release”, provided said controlled release, extended release or pulsatile release dosage forms are not delayed onset dosage forms.

In some embodiments, one or more, or substantially all, or all of the embodiments and specifications showing benefit or a difference for modified release delayed onset dosage forms of oral buprenorphine (e.g., delayed onset, rapid release; delayed onset, pulsatile release; delayed onset, extended release) over “sublingual”, “buccal”, “lingual” or “oromucosal” buprenorphine, the phrase “sublingual”, “buccal”, “lingual” or “oromucosal” may be substituted with “controlled release”, “extended release” or “pulsatile release”, provided said controlled release, extended release or pulsatile release dosage forms are not delayed onset dosage forms.

In some embodiments, controlled release dosage forms of the present invention releases buprenorphine from the oral dosage form at a slower rate than immediate release formulations. In some preferred embodiments, extended release dosage forms and delayed onset, extended release dosage forms release buprenorphine at such a rate that plasma concentrations and/or therapeutic effects are maintained within the therapeutic range (above the minimum effective therapeutic concentration) but below toxic levels for intended duration (e.g., over a period of about 1 to about 170 hours, preferably over a period of time indicative of a Q3H, Q4H, Q6H, Q8H, Q12H, Q16H, Q18H, Q24H, Q36H, Q48H or Q72H administration, more preferably over a period of time indicative of a Q12H, Q24H or Q48H administration). In some preferred embodiments, the extended release formulations of the present invention provide therapeutic effects for a duration that is longer or substantially longer than the duration of meaningful or detectable plasma concentrations of buprenorphine.

When applied to the present invention, present invention, the term “immediate release”, “immediate release dosage forms”, “immediate release composition”, “immediate release tablet”, “immediate release capsule”, “immediate release formulation”, immediate release forms” and the like is a dosage form which is formulated to release the active drug from the dosage form immediately (i.e., without an attempt to delay or prolong the release of the active drug from the dosage form as is the case, for example, with extended release dosage forms) or a dosage form which allows the drug to dissolve in the gastrointestinal contents, with no intention of delaying or prolonging the dissolution or absorption of the drug). Immediate release dosage forms may be in any form, including tablet, capsule, solution, suspension, powder, micronized, granulated etc. When applied to buprenorphine dosage forms of the invention, unless further modified to alter the meaning, “immediate release” refers to oral dosage forms. In the absence of a commercially available oral immediate release buprenorphine product, an available parenteral formulation of buprenorphine or a salt thereof may be used orally, or a solution of buprenorphine or a salt thereof may be prepared or an immediate release tablet may be prepared, or a sublingual or buccal formulation may be given orally for the purpose of in vivo testing requiring immediate release buprenorphine. Alternatively, an immediate release formulation of buprenorphine may be prepared by encapsulating liquid or uncompressed solid buprenorphine, or by compressing buprenorphine into tablet form without excipients or material that impart a delay or retardation to its release. Immediate release dosage forms generally disintegrate in ≦ about 0.5 hours or ≦ about 1 hour, and generally substantially or completely dissolve ii about 0.25, or ≦ about 0.5, or ≦ about 0.75, or ≦ about 1 or ≦ about 1.25, or ≦ about 1.5, or ≦ about 1.75, or ≦ about 2 hours, when measured by the recommended or appropriate USP compendial methods (for example some dosage forms may be tested by USP Basket Method or USP Paddle Method at 100 rpm in 900 mL of water at 37° C.).

In the absence of a commercially available immediate release sublingual formulation of buprenorphine listed in FDA's Orange Book, alternative sublingual formulations may be substituted (for example, commercially available sublingual formulations approved by the EMEA or available in the European Union, or sublingual formulations listed in Martindale: The Complete Drug Reference, 35th Edition (or more recent), Pharmaceutical Press).

For purposes of the invention, the oral controlled release formulations disclosed herein and the oral immediate release control formulations are dose proportional. In such formulations, the pharmacokinetic parameters (e.g., AUC and Cₘₐₓ) generally increase linearly from one dosage strength to another. Therefore the pharmacokinetic parameters of a particular dose can be inferred from the parameters of a different dose of the same formulation.

As used herein with respect to the buprenorphine dosage form of the invention, “duodenal release” and “duodenal delivery” are interchangeable and refer to in vivo release of all, substantially all or most buprenorphine from the dosage form into the portion of gastrointestinal tract distal to the stomach. In some embodiments, duodenal release or duodenal delivery dosage forms of the invention provide in vivo release of all, substantially all or most buprenorphine from the dosage form rapidly upon reaching the portion of gastrointestinal tract distal to the stomach. In other embodiments, duodenal release and duodenal delivery dosage forms of the invention provide in vivo release of all, substantially all or most buprenorphine from the dosage form slowly (e.g., sustained release or extended release) upon reaching the portion of gastrointestinal tract distal to the stomach.

As used herein with respect to the buprenorphine dosage form of the invention, “jejunal release” and “jejunal delivery” are interchangeable, and refer to in vivo release of all, substantially all or most buprenorphine from the dosage form into the portion of gastrointestinal tract distal to the duodenum. In some embodiments, jejunal release” or “jejunal delivery dosage forms of the invention provide in vivo release of all, substantially all or most buprenorphine from the dosage form rapidly upon reaching the portion of gastrointestinal tract distal to the duodenum. In other embodiments, duodenal release and duodenal delivery dosage forms of the invention provide in vivo release of all, substantially all or most buprenorphine from the dosage form slowly (e.g., sustained release or extended release) upon reaching the portion of gastrointestinal tract distal to the duodenum.

As used herein with respect to the buprenorphine dosage form of the invention, “ileal release” and “ileal delivery” are interchangeable and refer to in vivo release of all, substantially all or most buprenorphine from the dosage form into the portion of gastrointestinal tract distal to the jejunum. In some embodiments, ileal release or ileal delivery dosage forms of the invention provide in vivo release of all, substantially all or most buprenorphine from the dosage form rapidly upon reaching the portion of gastrointestinal tract distal to the jejunum. In other embodiments, ileal release and ileal delivery dosage forms of the invention provide in vivo release of all, substantially all or most buprenorphine from the dosage form slowly (e.g., sustained release or extended release) upon reaching the portion of gastrointestinal tract distal to the jejunum.

As used herein with respect to the buprenorphine dosage form of the invention, “ileo-colonic release” and “ileo-colonic delivery” are interchangeable and are interchangeable and refer to in vivo release of all, substantially all or most buprenorphine from the dosage form into the portion of gastrointestinal tract distal to the jejunum and/or distal to the ileum. In some embodiments, ileo-colonic release or ileo-colonic delivery dosage forms of the invention provide in vivo release of all, substantially all or most buprenorphine from the dosage form rapidly upon reaching the portion of gastrointestinal tract distal to the jejunum. In other embodiments, ileo-colonic release and ileo-colonic delivery dosage forms of the invention provide in vivo release of all, substantially all or most buprenorphine from the dosage form slowly (e.g., sustained release or extended release) upon reaching the portion of gastrointestinal tract distal to the jejunum.

As used herein with respect to the buprenorphine dosage form of the invention, “colonic release” and “colonic delivery” are interchangeable and refer to in vivo release of all, substantially all or most buprenorphine from the dosage form into the portion of gastrointestinal tract distal to the ileum. In some embodiments, colonic release and colonic delivery dosage forms of the invention provide in vivo release of all, substantially all or most buprenorphine from the dosage form rapidly upon reaching the portion of gastrointestinal tract distal to the ileum. In other embodiments, colonic release and colonic delivery dosage forms of the invention provide in vivo release of all, substantially all or most buprenorphine from the dosage form slowly (e.g., sustained release or extended release) upon reaching the portion of gastrointestinal tract distal to the ileum.

In some preferred embodiments, the dosage form provides an oral pharmaceutical composition for the treatment of a buprenorphine responsive medical condition comprising a therapeutically effective amount of buprenorphine or pharmaceutically acceptable salts thereof or mixture thereof; said dosage form providing an in-vitro buprenorphine release rate, when measured by the USP Basket or Paddle Method at 100 rpm in 900 mL of distilled water at 37° C. which is substantially pH dependent in that a difference, at 1, or 1.5, or 2, or 2.5, or 3 hours, between the amount of buprenorphine released at a pH of ≦0.5, or ≦1, or ≦1.5, or ≦2, or ≦2.5, or ≦3, or ≦3.5, or ≦4, or ≦4.5, or ≦5, or ≦5.5 and an amount released at a pH of or ≧5.8, or ≧6, or ≧6.2, or ≧6.4, or ≧6.6, or ≧6.8, or ≧7, or ≧7.1, or ≧7.2, or ≧7.3, or ≧7.4, or ≧7.5, or ≧7.6, or ≧7.7, or ≧7.8, or ≧7.9, or ≧8, is greater than about 20%, 25%, 35%, 50%, 60%, 75%, 80%, 90%, 100%, 125%, 150%, 175%, 200%, 225%, 250%, 275%, 300%, 350%, 400%, 450%, 500%, 600%, 700%, 800%, 900%, 1000%, or 1200%, 1500%, 2000%, 3000%, 4000%%, 5000%, or 6000%.

In some preferred embodiments, the dosage form provides an oral pharmaceutical composition for the treatment of a buprenorphine responsive medical condition comprising a therapeutically effective amount of buprenorphine or pharmaceutically acceptable salts thereof or mixture thereof; said dosage form providing duodenal delivery, jejunal delivery, ileal delivery, ileo-colonic delivery or colonic delivery; said dosage form providing an in-vitro buprenorphine release rate, when measured by the USP Basket or Paddle Method at 100 rpm in 900 mL of distilled water at 37° C. which is substantially pH dependent in that a difference, at 1, or 1.5, or 2, or 2.5, or 3 hours, between the amount of buprenorphine released at a pH of ≦0.5, or ≦1, or ≦1.5, or ≦2, or ≦2.5, or ≦3, or ≦3.5, or ≦4, or ≦4.5, or ≦5, or ≦5.5 and an amount released at a pH of ≧5.8, or ≧6, or ≧6.2, or ≧6.4, or ≧6.6, or ≧6.8, or ≧7, or ≧7.1, or ≧7.2, or ≧7.3, or ≧7.4, or ≧7.5, or ≧7.6, or ≧7.7, or ≧7.8, or ≧7.9, or ≧8, is greater than about 20%, 25%, 35%, 50%, 60%, 75%, 80%, 90%, 100%, 125%, 150%, 175%, 200%, 225%, 250%, 275%, 300%, 350%, 400%, 450%, 500%, 600%, 700%, 800%, 900%, 1000%, or 1200%, 1500%, 2000%, 3000%, 4000%%, 5000%, or 6000%.

In some preferred embodiments, the dosage form provides an oral pharmaceutical composition for the treatment of a buprenorphine responsive medical condition comprising a therapeutically effective amount of buprenorphine or pharmaceutically acceptable salts thereof or mixture thereof, and a controlled release material; said dosage form providing an in-vitro buprenorphine release rate, when measured by the USP Basket or Paddle Method at 100 rpm in 900 mL of distilled water at 37° C. which is substantially pH dependent in that a difference, at 1, or 1.5, or 2, or 2.5, or 3 hours, between the amount of buprenorphine released at a pH of ≦0.5, or ≦1, or ≦1.5, or ≦2, or ≦2.5, or ≦3, or ≦3.5, or ≦4, or ≦4.5, or ≦5, or ≦5.5 and an amount released at a pH of ≧5.8, or ≧6, or ≧6.2, or ≧6.4, or ≧6.6, or ≧6.8, or ≧7, or ≧7.1, or ≧7.2, or ≧7.3, or ≧7.4, or ≧7.5, or ≧7.6, or ≧7.7, or ≧7.8, or ≧7.9, or ≧8, is greater than about 25%, 35%, 50%, 60%, 75%, 80%, 90%, 100%, 125%, 150%, 175%, 200%, 225%, 250%, 275%, 300%, 350%, 400%, 450%, 500%, 600%, 700%, 800%, 900%, 1000%, or 1200%, 1500%, 2000%, 3000%, 4000%%, 5000%, or 6000%.

In some preferred embodiments, the dosage form provides an oral pharmaceutical composition for the treatment of a buprenorphine responsive medical condition comprising a therapeutically effective amount of buprenorphine or pharmaceutically acceptable salts thereof or mixture thereof, and a controlled release material; said dosage form providing duodenal delivery, jejunal delivery, ileal delivery, ileo-colonic delivery or colonic delivery; said dosage form providing an in-vitro buprenorphine release rate, when measured by the USP Basket or Paddle Method at 100 rpm in 900 mL of distilled water at 37° C. which is substantially pH dependent in that a difference, at 1, or 1.5, or 2, or 2.5, or 3 hours, between the amount of buprenorphine released at a pH of ≦0.5, or ≦1, or ≦1.5, or ≦2, or ≦2.5, or ≦3, or ≦3.5, or ≦4, or ≦4.5, or ≦5, or ≦5.5 and an amount released at a pH of ≧5.8, or ≧6, or ≧6.2, or ≧6.4, or ≧6.6, or ≧6.8, or ≧7, or ≧7.1, or ≧7.2, or ≧7.3, or ≧7.4, or ≧7.5, or ≧7.6, or ≧7.7, or ≧7.8, or ≧7.9, or ≧8, is greater than about 25%, 35%, 50%, 60%, 75%, 80%, 90%, 100%, 125%, 150%, 175%, 200%, 225%, 250%, 275%, 300%, 350%, 400%, 450%, 500%, 600%, 700%, 800%, 900%, 1000%, or 1200%, 1500%, 2000%, 3000%, 4000%%, 5000%, or 6000%.

In some preferred embodiments, the dosage form provides an oral pharmaceutical composition for the treatment of a buprenorphine responsive medical condition comprising a therapeutically effective amount of buprenorphine or pharmaceutically acceptable salts thereof or mixture thereof, and a controlled release material; said dosage form, following dissolution (pretreatment) with USP Basket or Paddle Method at 100 rpm in 900 mL of 0.1N HCl for two hours at 37° C., providing an in-vitro release rate of buprenorphine by weight, when measured at about 5, 10, 15, 20, 25, 30, 40, 50, or 60 minutes, 1, 1.5, 2, 2.5, 3, 4, 5 6, 7, 8, 10, 12, 14, 16, 18, 20, 24 or 30 hours by the USP Basket or Paddle Method at 100 rpm in 900 mL distilled water (time=0 hour begins here) at 37° C. at a pH between 4.5 and 8: (i) of less than about 1%, 2%, 3%, 4%, 5%, 10%, 15%, 20%, 25%, 30%, 40%, 50%, 60%, 70% 80%, 90% or 100%; or (ii) of less than about 1% to about 20% or less than about 2% to about 20% or less than about 5% to about 20% or less than about 8% to about 20% or less than about 10% to about 20% or less than about 12% to about 20% or less than about 15% to about 20% or less than about 1% to about 50% or less than about 2% to about 50% or less than about 5% to about 50% or less than about 10% to about 50% or less than about 15% to about 50% or less than about 20% to about 50% or less than about 30% to about 50% or less than about 40% to about 50% or less than about 1% to about 60% or less than about 2% to about 60% or less than about 5% to about 60% or less than about 10% to about 60% or less than about 15% to about 60% or less than about 20% to about 60% or less than about 30% to about 60% or less than about 40% to about 60% or less than about 1% to about 70% or less than about 2% to about 70% or less than about 5% to about 70% or less than about 10% to about 70% or less than about 15% to about 70% or less than about 20% to about 70% or less than about 30% to about 70% or less than about 50% to about 70% or less than about 1% to about 80% or less than about 2% to about 80% or less than about 5% to about 80% or less than about 10% to about 80% or less than about 15% to about 80% or less than about 20% to about 80% or less than about 30% to about 80% or less than about 40% to about 80% or less than about 1% to about 90% or less than about 2% to about 90% or less than about 5% to about 90% or less than about 10% to about 90% or less than about 15% to about 90% or less than about 20% to about 90% or less than about 30% to about 90% or less than about 40% to about 90% or about 60% to about 90% or less than about 70% to about 90% or less than about 80% to about 90%, or more than about 1%, or more than about 5%, or more than about 10%, or more than about 15%, or more than about 20%, or more than about 30%, or more than about 40%, or more than about 50%, or more than about 55%, or more than about 60%, or more than about 70%, or more than about 80%, or more than about 85%, or more than about 90%, or more than about 95%, or more than 99%; or (iii) of ≧0.1%, or ≧0.5%, or ≧1%, or ≧5%, ≧10%, or ≧20%, or ≧30%, or ≧40%, or ≧50%, or ≧60%, or ≧70%, or ≧80%, or ≧90%, or about 100%.

In some preferred embodiments, the dosage form provides an oral pharmaceutical composition for the treatment of a buprenorphine responsive medical condition comprising a therapeutically effective amount of buprenorphine or pharmaceutically acceptable salts thereof or mixture thereof, and a controlled release material; said dosage form providing duodenal release, jejunal release, ileal release, ileo-colonic release or colonic release; said dosage form, following dissolution (pretreatment) with USP Basket or Paddle Method at 100 rpm in 900 mL of 0.1N HCl for two hours at 37° C., providing an in-vitro release rate of buprenorphine by weight, when measured at about 5, 10, 15, 20, 25, 30, 40, 50, or 60 minutes, 1, 1.5, 2, 2.5, 3, 4, 5 6, 7, 8, 10, 12, 14, 16, 18, 20, 24 or 30 hours by the USP Basket or Paddle Method at 100 rpm in 900 mL distilled water (time=0 hour begins here) at 37° C. at a pH between 4.5 and 8: (i) of less than about 1%, 2%, 3%, 4%, 5%, 10%, 15%, 20%, 25%, 30%, 40%, 50%, 60%, 70% 80%, 90% or 100%; or (ii) of less than about 1% to about 20% or less than about 2% to about 20% or less than about 5% to about 20% or less than about 8% to about 20% or less than about 10% to about 20% or less than about 12% to about 20% or less than about 15% to about 20% or less than about 1% to about 50% or less than about 2% to about 50% or less than about 5% to about 50% or less than about 10% to about 50% or less than about 15% to about 50% or less than about 20% to about 50% or less than about 30% to about 50% or less than about 40% to about 50% or less than about 1% to about 60% or less than about 2% to about 60% or less than about 5% to about 60% or less than about 10% to about 60% or less than about 15% to about 60% or less than about 20% to about 60% or less than about 30% to about 60% or less than about 40% to about 60% or less than about 1% to about 70% or less than about 2% to about 70% or less than about 5% to about 70% or less than about 10% to about 70% or less than about 15% to about 70% or less than about 20% to about 70% or less than about 30% to about 70% or less than about 50% to about 70% or less than about 1% to about 80% or less than about 2% to about 80% or less than about 5% to about 80% or less than about 10% to about 80% or less than about 15% to about 80% or less than about 20% to about 80% or less than about 30% to about 80% or less than about 40% to about 80% or less than about 1% to about 90% or less than about 2% to about 90% or less than about 5% to about 90% or less than about 10% to about 90% or less than about 15% to about 90% or less than about 20% to about 90% or less than about 30% to about 90% or less than about 40% to about 90% or about 60% to about 90% or less than about 70% to about 90% or less than about 80% to about 90%, or more than about 1%, or more than about 5%, or more than about 10%, or more than about 15%, or more than about 20%, or more than about 30%, or more than about 40%, or more than about 50%, or more than about 55%, or more than about 60%, or more than about 70%, or more than about 80%, or more than about 85%, or more than about 90%, or more than about 95%, or more than 99%; or (iii) of ≧0.1%, or ≧0.5%, or ≧1%, or ≧5%, ≧10%, or ≧20%, or ≧30%, or ≧40%, or ≧50%, or ≧60%, or ≧70%, or ≧80%, or ≧90%, or about 100%.

In some embodiments of the invention, pH adjustments of the dissolution media may be achieved by adjustment as required with hydrochloric acid or sodium hydroxide. In some embodiments of the invention, pH adjustments of the dissolution media may be achieved with other pharmaceutical excipients, including acids, bases and buffers known in the art.

In some preferred embodiments, the dosage form provides an oral pharmaceutical composition for the treatment of a buprenorphine responsive medical condition comprising a therapeutically effective amount of buprenorphine or pharmaceutically acceptable salts thereof or mixture thereof, and a controlled release material; said dosage form, following dissolution (pretreatment) with USP Basket or Paddle Method at 100 rpm in 900 mL of 0.1N HCl for two hours at 37° C., and then following further dissolution (pretreatment) with USP Basket or Paddle Method at 100 rpm in 900 mL distilled water at any pH of between 2 and 4 for a further time of up to two hours at 37° C., providing an in-vitro release rate by weight of buprenorphine, when measured by the USP Basket or Paddle Method at 100 rpm in 900 mL distilled water (time=0 hour begins here) at 37° C. at any pH between 4.5 and 8 at 37° C. (measured at about 5, about 10, about 15, about 20, about 25, about 30, about 35, about 40, about 45, about 50, about 55 or about 60 minutes, or about 2 hours, or about 3 hours, or about 4 hours, or about 5 hours, or about 6 hours, or about 7 hours, or about 8 hours, or about 10 hours, or about 12 hours, or about 14 hours, or about 16 hours, or about 18 hours, or about 20 hours, or about 24 hours, or about 30 hours): (i) of less than about 1% to about 20% or less than about 2% to about 20% or less than about 5% to about 20% or less than about 8% to about 20% or less than about 10% to about 20% or less than about 12% to about 20% or less than about 15% to about 20% or less than about 1% to about 50% or less than about 2% to about 50% or less than about 5% to about 50% or less than about 10% to about 50% or less than about 15% to about 50% or less than about 20% to about 50% or less than about 30% to about 50% or less than about 40% to about 50% or less than about 1% to about 60% or less than about 2% to about 60% or less than about 5% to about 60% or less than about 10% to about 60% or less than about 15% to about 60% or less than about 20% to about 60% or less than about 30% to about 60% or less than about 40% to about 60% or less than about 1% to about 70% or less than about 2% to about 70% or less than about 5% to about 70% or less than about 10% to about 70% or less than about 15% to about 70% or less than about 20% to about 70% or less than about 30% to about 70% or less than about 50% to about 70% or less than about 1% to about 80% or less than about 2% to about 80% or less than about 5% to about 80% or less than about 10% to about 80% or less than about 15% to about 80% or less than about 20% to about 80% or less than about 30% to about 80% or less than about 40% to about 80% or less than about 1% to about 90% or less than about 2% to about 90% or less than about 5% to about 90% or less than about 10% to about 90% or less than about 15% to about 90% or less than about 20% to about 90% or less than about 30% to about 90% or less than about 40% to about 90% or about 60% to about 90% or less than about 70% to about 90% or less than about 80% to about 90%, or more than about 1%, or more than about 5%, or more than about 10%, or more than about 15%, or more than about 20%, or more than about 30%, or more than about 40%, or more than about 50%, or more than about 55%, or more than about 60%, or more than about 70%, or more than about 80%, or more than about 85%, or more than about 90%, or more than about 95%, or more than 99%, or about 100%; or (ii) of ≧0.1%, or ≧0.5%, or ≧1%, or ≧5%, ≧10%, or ≧20%, or ≧30%, or ≧40%, or ≧50%, or ≧60%, or ≧70%, or ≧80%, or ≧90%, or about 100. In some embodiments, the foregoing dosage form provides duodenal delivery, jejunal delivery, ileal delivery, ileo-colonic delivery or colonic delivery.

In some preferred embodiments, the dosage form provides an oral pharmaceutical composition for the treatment of a buprenorphine responsive medical condition comprising a therapeutically effective amount of buprenorphine or pharmaceutically acceptable salts thereof or mixture thereof, and a controlled release material; said dosage form, following dissolution (pretreatment) with USP Basket or Paddle Method at 100 rpm in 900 mL of 0.1N HCl for two hours at 37° C., and then following further dissolution (pretreatment) with USP Basket or Paddle Method at 100 rpm in 900 mL distilled water at any pH of between 2 and 4 for a further time of up to two hours at 37° C., providing an in-vitro release rate by weight of buprenorphine, when measured by the USP Basket or Paddle Method at 100 rpm in 900 mL distilled water (time=0 hour begins here) at 37° C. at any pH between 4.5 and 8 at 37° C.: (i) from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours; or (ii) from 0% to about 40% at 1 hour, from about 5% to about 55% at 2 hours, from about 10% to about 60% at 4 hours, from about 15% to about 70% at 6 hours, from about 25% to about 80% at 9 hours, and greater than about 50% at 12 hours. In some embodiments, the foregoing dosage form provides duodenal delivery, jejunal delivery, ileal delivery, ileo-colonic delivery or colonic delivery.

In some preferred embodiments, the dosage form provides an oral pharmaceutical composition for the treatment of a buprenorphine responsive medical condition comprising a therapeutically effective amount of buprenorphine or pharmaceutically acceptable salts thereof or mixture thereof; said dosage form, following dissolution (pretreatment) with USP Basket or Paddle Method at 100 rpm in 900 mL of 0.1N HCl for two hours at 37° C., providing an in-vitro release rate by weight of buprenorphine, when measured by the USP Basket or Paddle Method at 100 rpm in 900 mL distilled water (time=0 hour begins here) at 37° C. at any pH between 4.5 and 8 at 37° C.: (1) between 0% to about 47.5% at 1 hour, between about 10% to about 65% at 2 hours, between about 15% to about 70% at 4 hours, between about 25% to about 77.5% at 6 hours, between about 35% to about 87.5% at 9 hours, and greater than about 65% at 12 hours; or (2) between about 10% to about 65% at 4 hours, between about 20% to about 70% at 8 hours, between about 25% to about 80% at 12 hours, between about 35% to about 95% at 18 hours, and greater than about 65% at 24 hours; or (3) between 0% to about 60% at 1 hour, between about 0% and about 80% at 2 hours, between about 3% and about 95% at 4 hours and between about 10% and about 100% at 8 hours; or (4) between about 10% and about 65% at 1 hour, between about 20% and about 75% at 2 hours, between about 30% and about 95% at 4 hours and between about 40% and about 100% at 8 hours; or (5) between about 10% to about 65% at 2 hours, between about 15% to about 70% at 4 hours, between about 25% to about 77.5% at 6 hours, between about 35% to about 87.5% at 9 hours, and greater than about 65% at 12 hours; or (6) between about 5% and about 50% at 1 hour, between about 10% and about 75% at 2 hours, between about 20% and about 95% at 4 hours, between about 40% and about 100% at 8 hours, greater than about 50% at 12 hours, greater than about 70% at 18 hours, and greater than about 80% at 24 hours; or (7) between about 5% and about 50% at 1 hour, between about 10% and about 75% at 2 hours, between about 20% and about 95% at 4 hours, between about 40% and about 100% at 8 hours, greater than about 50% at 12 hours, greater than about 70% at 18 hours, and greater than about 80% at 24 hours; or (8) between 0% to about 30% at 1 hour, between about 10% to about 65% at 4 hours, between about 20% to about 70% at 8 hours, between about 25% to about 80% at 12 hours, between about 35% to about 95% at 18 hours, and greater than about 65% at 24 hours; or (9) between 0% to about 50% at 1 hour, between about 0% and about 75% at 2 hours, between about 3% and about 95% at 4 hours, between about 10% and about 100% at 8 hours, between about 25% and about 100% at 12 hours, between about 30% and about 100% at 16 hours, between about 50% and about 100% at 24 hours, and greater than about 80% at 36 hours; or (10) between about 20% and about 50% at 1 hour, between about 40% and about 75% at 2 hours, between about 60% and about 95% at 4 hours, between about 80% and about 100% at 8 hours and between about 90% and about 100% at 12 hours; or (11) between 0% to about 50% at 1 hour, between about 0% and about 75% at 2 hours, between about 10% and about 95% at 4 hours, between about 35% and about 100% at 8 hours, between about 55% and about 100% at 12 hours, between about 70% to about 100% at 16 hours, and greater than about 90% at 24 hours; or (12) between 0% to about 30% at 1 hour, between about 0% and about 45% at 2 hours, between about 3% and about 55% at 4 hours, between about 10% and about 65% at 8 hours, between about 20% and about 75% at 12 hours, between about 30% to about 88% at 16 hours, between about 50% and about 100% hours at 24 hours and greater than 80% at 36 hours; or (13) between 0% to about 50% at 1 hour, between about 0% and about 75% at 2 hours, between about 3% and about 95% at 4 hours, between about 10% and about 100% at 8 hours, between about 20% and about 100% at 12 hours, between about 30% to about 100% at 16 hours, between about 50% and about 100% hours at 24 hours and greater than 80% at 36 hours; or (14) between about 15% and about 25% at 1 hour, between about 25% and about 35% at 2 hours, between about 30% and about 45% at 4 hours, between about 40% and about 60% at 8 hours, between about 55% and about 70% at 12 hours and between about 60% to about 75% at 16 hours; or (15) between 0% to about 60% at 1 hour, between about 0% and about 80% at 2 hours, between about 3% and about 95% at 4 hours and between about 10% and about 100% at 8 hours; or (16) between 0% and about 10% at 1 hour, between about 0% and about 20% at 2 hours, between about 2% and about 80% at 4 hours and between about 5% and about 100% at 8 hours; or (17) between 0% and about 20% at 1 hour, between about 0% and about 40% at 2 hours, between about 0% and about 80% at 4 hours and between about 2% and about 100% at 8 hours; or (18) between 0% and about 40% at 1 hour, between about 0% and about 60% at 2 hours, between about 5% and about 85% at 4 hours and between about 5% and about 90% at 8 hours and greater than 20% at 12 hours; or (19) between 0% and about 50% at 1 hour, between about 0% and about 50% at 2 hours, between about 10% and about 90% at 4 hours and between about 15% and about 90% at 8 hours and greater than 30% at 12 hours; or (20) between 0% and about 70% at 1 hour, between about 0% and about 70% at 2 hours, between about 10% and about 75% at 4 hours and between about 15% and about 90% at 8 hours and greater than 30% at 12 hours; or (21) between about 10% and about 65% at 1 hour, between about 20% and about 75% at 2 hours, between about 30% and about 95% at 4 hours and between about 40% and about 100% at 8 hours; or (22) between 2% and about 70% at 1 hour, between about 5% and about 80% at 2 hours, between about 10% and about 90% at 4 hours and between about 20% and about 100% at 8 hours; or (23) between 5% and about 60% at 1 hour, between about 10% and about 75% at 2 hours, between about 15% and about 85% at 4 hours and between about 30% and about 100% at 8 hours; or (24) between 20% and about 70% at 1 hour, between about 20% and about 75% at 2 hours, between about 20% and about 90% at 4 hours and between about 40% and about 100% at 8 hours; or (25) between 30% and about 80% at 1 hour, between about 40% and about 85% at 2 hours, between about 40% and about 90% at 4 hours and between about 60% and about 100% at 8 hours; or (26) between 1% and about 20% at 1 hour, between about 5% and about 20% at 2 hours, between about 10% and about 40% at 4 hours and between about 20% and about 40% at 8 hours and greater than 40% at 12 hours; or (27) between 0% to about 47.5% at 1 hour, between about 10% to about 65% at 2 hours, between about 15% to about 70% at 4 hours, between about 25% to about 77.5% at 6 hours, between about 35% to about 87.5% at 9 hours, and greater than about 65% at 12 hours; or (28) between 0% to about 30% at 1 hour, between about 5% to about 45% at 2 hours, between about 10% to about 60% at 4 hours, between about 15% to about 70% at 6 hours, between about 25% to about 80% at 9 hours, and greater than about 50% at 12 hours; or (29) between 0% to about 20% at 1 hour, between about 2% to about 35% at 2 hours, between about 5% to about 50% at 4 hours, between about 10% to about 60% at 6 hours, between about 15% to about 70% at 9 hours, and greater than about 40% at 12 hours; or (30) between 0% to about 10% at 1 hour, between about 1% to about 30% at 2 hours, between about 5% to about 40% at 4 hours, between about 10% to about 60% at 6 hours, between about 15% to about 70% at 9 hours, and greater than about 40% at 12 hours; or (31) between 0% to about 5% at 1 hour, between about 0% to about 10% at 2 hours, between about 2% to about 20% at 4 hours, between about 5% to about 30% at 6 hours, between about 10% to about 40% at 9 hours, and greater than about 30% at 12 hours; or (32) between 0% to about 50% at 1 hour, between about 15% to about 70% at 2 hours, between about 20% to about 75% at 4 hours, between about 30% to about 80% at 6 hours, between about 30% to about 90% at 9 hours, and greater than about 70% at 12 hours; or (33) between 0% to about 60% at 1 hour, between about 15% to about 80% at 2 hours, between about 25% to about 85% at 4 hours, between about 35% to about 90% at 6 hours, between about 40% to about 90% at 9 hours, and greater than about 80% at 12 hours; (34) between 0% to about 70% at 1 hour, between about 20% to about 80% at 2 hours, between about 25% to about 80% at 4 hours, between about 35% to about 80% at 6 hours, between about 40% to about 80% at 9 hours, and greater than about 60% at 12 hours; or (35) between 0% to about 75% at 1 hour, between about 30% to about 80% at 2 hours, between about 35% to about 90% at 4 hours, between about 50% to about 90% at 6 hours, between about 55% to about 95% at 9 hours, and greater than about 70% at 12 hours; or (36) between about 5% and about 50% at 1 hour, between about 10% and about 75% at 2 hours, between about 20% and about 95% at 4 hours, between about 40% and about 100% at 8 hours, greater than about 50% at 12 hours, greater than about 70% at 18 hours, and greater than about 80% at 24 hours; or (37) between 2% and about 50% at 1 hour, between about 5% and about 75% at 2 hours, between about 15% and about 75% at 4 hours, between about 30% and about 90% at 8 hours, greater than about 40% at 12 hours, greater than about 60% at 18 hours, and greater than about 70% at 24 hours; or (38) between 1% and about 40% at 1 hour, between about 2% and about 60% at 2 hours, between about 10% and about 65% at 4 hours, between about 20% and about 80% at 8 hours, greater than about 30% at 12 hours, greater than about 40% at 18 hours, and greater than about 60% at 24 hours; or (39) between 5% and about 60% at 1 hour, between about 15% and about 80% at 2 hours, between about 25% and about 95% at 4 hours, between about 45% and about 100% at 8 hours, greater than about 60% at 12 hours, greater than about 80% at 18 hours, and greater than about 90% at 24 hours; or (40) between 10% and about 65% at 1 hour, between about 20% and about 85% at 2 hours, between about 30% and about 100% at 4 hours, between about 60% and about 100% at 8 hours, greater than about 70% at 12 hours, greater than about 90% at 18 hours, and greater than about 95% at 24 hours; or (41) between 0% to about 30% at 1 hour, between about 10% to about 65% at 4 hours, between about 20% to about 70% at 8 hours, between about 25% to about 80% at 12 hours, between about 35% to about 95% at 18 hours, and greater than about 65% at 24 hours; or (42) between 0% to about 20% at 1 hour, between about 5% to about 50% at 4 hours, between about 10% to about 60% at 8 hours, between about 15% to about 70% at 12 hours, between about 25% to about 90% at 18 hours, and greater than about 55% at 24 hours; or (43) between 0% to about 10% at 1 hour, between about 5% to about 40% at 4 hours, between about 8% to about 50% at 8 hours, between about 10% to about 60% at 12 hours, between about 22% to about 80% at 18 hours, and greater than about 45% at 24 hours; or (44) between 0% to about 35% at 1 hour, between about 15% to about 70% at 4 hours, between about 25% to about 75% at 8 hours, between about 30% to about 85% at 12 hours, between about 40% to about 100% at 18 hours, and greater than about 75% at 24 hours; or (45) between 0% to about 40% at 1 hour, between about 20% to about 70% at 4 hours, between about 30% to about 80% at 8 hours, between about 35% to about 90% at 12 hours, between about 45% to about 100% at 18 hours, and greater than about 80% at 24 hours; or (46) between 0% to about 45% at 1 hour, between about 25% to about 75% at 4 hours, between about 35% to about 85% at 8 hours, between about 40% to about 90% at 12 hours, between about 50% to about 100% at 18 hours, and greater than about 90% at 24 hours; or (47) between 0% to about 50% at 1 hour, between about 30% to about 80% at 4 hours, between about 40% to about 90% at 8 hours, between about 45% to about 95% at 12 hours, between about 60% to about 100% at 18 hours, and greater than about 95% at 24 hours; or (48) between 0% to about 60% at 1 hour, between about 40% to about 80% at 4 hours, between about 45% to about 90% at 8 hours, between about 50% to about 100% at 12 hours, between about 70% to about 100% at 18 hours, and greater than about 80% at 24 hours; or (49) between 0% and about 50% at 1 hour, between about 0% and about 75% at 2 hours, between about 3% and about 95% at 4 hours, between about 10% and about 100% at 8 hours, between about 25% and about 100% at 12 hours, between about 30% and about 100% at 16 hours, between about 50% and about 100% at 24 hours, and greater than about 80% at 36 hours; or (50) between 0% and about 40% at 1 hour, between about 0% and about 65% at 2 hours, between about 2% and about 85% at 4 hours, between about 8% and about 90% at 8 hours, between about 20% and about 95% at 12 hours, between about 25% and about 95% at 16 hours, between about 40% and about 90% at 24 hours, and greater than about 70% at 36 hours; or (51) between 0% and about 30% at 1 hour, between about 0% and about 50% at 2 hours, between about 1% and about 75% at 4 hours, between about 5% and about 80% at 8 hours, between about 10% and about 85% at 12 hours, between about 15% and about 90% at 16 hours, between about 30% and about 80% at 24 hours, and greater than about 70% at 36 hours; or (52) between 0% and about 60% at 1 hour, between about 0% and about 80% at 2 hours, between about 5% and about 100% at 4 hours, between about 15% and about 100% at 8 hours, between about 35% and about 100% at 12 hours, between about 40% and about 100% at 16 hours, between about 60% and about 100% at 24 hours, and greater than about 85% at 36 hours; or (53) between 0% and about 65% at 1 hour, between about 0% and about 85% at 2 hours, between about 10% and about 100% at 4 hours, between about 20% and about 100% at 8 hours, between about 40% and about 100% at 12 hours, between about 50% and about 100% at 16 hours, between about 70% and about 100% at 24 hours, and greater than about 90% at 36 hours; or (54) between 0% and about 70% at 1 hour, between about 0% and about 90% at 2 hours, between about 20% and about 100% at 4 hours, between about 30% and about 100% at 8 hours, between about 50% and about 100% at 12 hours, between about 60% and about 100% at 16 hours, between about 80% and about 100% at 24 hours, and greater than about 95% at 36 hours; or (55) between 20% and about 50% at 1 hour, between about 40% and about 75% at 2 hours, between about 60% and about 95% at 4 hours, between about 80% and about 100% at 8 hours and between about 90% and about 100% at 12 hours; or (56) between 15% and about 45% at 1 hour, between about 35% and about 70% at 2 hours, between about 55% and about 90% at 4 hours, between about 75% and about 90% at 8 hours and between about 80% and about 95% at 12 hours; or (57) between 10% and about 40% at 1 hour, between about 30% and about 65% at 2 hours, between about 50% and about 85% at 4 hours, between about 70% and about 85% at 8 hours and between about 75% and about 90% at 12 hours; or (58) between 5% and about 35% at 1 hour, between about 25% and about 60% at 2 hours, between about 45% and about 80% at 4 hours, between about 65% and about 80% at 8 hours and between about 70% and about 85% at 12 hours; or (59) between 25% and about 55% at 1 hour, between about 45% and about 80% at 2 hours, between about 65% and about 95% at 4 hours, between about 85% and about 100% at 8 hours and between about 95% and about 100% at 12 hours; or (60) between 30% and about 60% at 1 hour, between about 50% and about 80% at 2 hours, between about 70% and about 95% at 4 hours, between about 90% and about 100% at 8 hours and between about 95% and about 100% at 12 hours; or (61) between 35% and about 60% at 1 hour, between about 50% and about 80% at 2 hours, between about 80% and about 95% at 4 hours, between about 90% and about 100% at 8 hours and between about 95% and about 100% at 12 hours; or (62) between 20% and about 40% at 1 hour, between about 40% and about 65% at 2 hours, between about 60% and about 85% at 4 hours, between about 70% and about 90% at 8 hours and between about 80% and about 100% at 12 hours; or (63) between 0% and about 50% at 1 hour, between about 0% and about 75% at 2 hours, between about 10% and about 95% at 4 hours, between about 35% and about 100% at 8 hours, between about 55% and about 100% at 12 hours, between about 70% to about 100% at 16 hours, and greater than about 90% at 24 hours; or (64) between 0% and about 40% at 1 hour, between about 0% and about 65% at 2 hours, between about 8% and about 85% at 4 hours, between about 30% and about 90% at 8 hours, between about 45% and about 100% at 12 hours, between about 60% to about 100% at 16 hours, and greater than about 80% at 24 hours; or (66) between 0% and about 30% at 1 hour, between about 0% and about 55% at 2 hours, between about 5% and about 75% at 4 hours, between about 20% and about 80% at 8 hours, between about 35% and about 100% at 12 hours, between about 50% to about 100% at 16 hours, and greater than about 70% at 24 hours; or (67) between 0% and about 20% at 1 hour, between about 0% and about 45% at 2 hours, between about 5% and about 65% at 4 hours, between about 10% and about 70% at 8 hours, between about 25% and about 80% at 12 hours, between about 40% to about 100% at 16 hours, and greater than about 60% at 24 hours; or (68) between 0% and about 60% at 1 hour, between about 0% and about 80% at 2 hours, between about 15% and about 95% at 4 hours, between about 40% and about 100% at 8 hours, between about 60% and about 100% at 12 hours, between about 75% to about 100% at 16 hours, and greater than about 90% at 24 hours; or (69) between 0% and about 65% at 1 hour, between about 0% and about 85% at 2 hours, between about 20% and about 90% at 4 hours, between about 45% and about 100% at 8 hours, between about 65% and about 100% at 12 hours, between about 80% to about 100% at 16 hours, and greater than about 90% at 24 hours; or (70) between 0% and about 40% at 1 hour, between about 0% and about 50% at 2 hours, between about 10% and about 80% at 4 hours, between about 25% and about 70% at 8 hours, between about 40% and about 80% at 12 hours, between about 60% to about 100% at 16 hours, and greater than about 90% at 24 hours; or (71) between 0% and about 30% at 1 hour, between about 0% and about 45% at 2 hours, between about 3% and about 55% at 4 hours, between about 10% and about 65% at 8 hours, between about 20% and about 75% at 12 hours, between about 30% to about 88% at 16 hours, between about 50% and about 100% hours at 24 hours and greater than 80% at 36 hours; or (72) between 0% and about 25% at 1 hour, between about 0% and about 40% at 2 hours, between about 2% and about 50% at 4 hours, between about 8% and about 60% at 8 hours, between about 10% and about 70% at 12 hours, between about 25% to about 80% at 16 hours, between about 45% and about 100% hours at 24 hours and greater than 75% at 36 hours; or (73) between 0% and about 20% at 1 hour, between about 0% and about 35% at 2 hours, between about 1% and about 45% at 4 hours, between about 5% and about 55% at 8 hours, between about 8% and about 65% at 12 hours, between about 20% to about 75% at 16 hours, between about 40% and about 100% hours at 24 hours and greater than 70% at 36 hours; or (74) between 0% and about 15% at 1 hour, between about 0% and about 30% at 2 hours, between about 0% and about 40% at 4 hours, between about 5% and about 50% at 8 hours, between about 8% and about 60% at 12 hours, between about 15% to about 70% at 16 hours, between about 35% and about 100% hours at 24 hours and greater than 60% at 36 hours; or (75) between 0% and about 10% at 1 hour, between about 0% and about 25% at 2 hours, between about 0% and about 35% at 4 hours, between about 5% and about 45% at 8 hours, between about 10% and about 50% at 12 hours, between about 10% to about 60% at 16 hours, between about 30% and about 90% hours at 24 hours and greater than 70% at 36 hours; or (76) between 0% and about 35% at 1 hour, between about 0% and about 50% at 2 hours, between about 5% and about 60% at 4 hours, between about 15% and about 70% at 8 hours, between about 25% and about 80% at 12 hours, between about 35% to about 90% at 16 hours, between about 55% and about 100% hours at 24 hours and greater than 85% at 36 hours; or (77) between 0% and about 40% at 1 hour, between about 0% and about 55% at 2 hours, between about 10% and about 65% at 4 hours, between about 20% and about 75% at 8 hours, between about 30% and about 85% at 12 hours, between about 40% to about 100% at 16 hours, between about 55% and about 100% hours at 24 hours and greater than 90% at 36 hours; or (78) between 0% and about 45% at 1 hour, between about 0% and about 60% at 2 hours, between about 15% and about 70% at 4 hours, between about 25% and about 80% at 8 hours, between about 35% and about 90% at 12 hours, between about 45% to about 100% at 16 hours, between about 60% and about 100% hours at 24 hours and greater than 60% at 36 hours; or (79) between 0% and about 50% at 1 hour, between about 5% and about 65% at 2 hours, between about 20% and about 75% at 4 hours, between about 30% and about 85% at 8 hours, between about 40% and about 95% at 12 hours, between about 50% to about 100% at 16 hours, between about 70% and about 100% hours at 24 hours and greater than 70% at 36 hours; or (80) between 0% and about 30% at 1 hour, between about 5% and about 40% at 2 hours, between about 10% and about 60% at 4 hours, between about 20% and about 70% at 8 hours, between about 30% and about 100% at 12 hours, between about 40% to about 100% at 16 hours, between about 60% and about 100% hours at 24 hours and greater than 90% at 36 hours; or (81) between 0% and about 30% at 1 hour, between about 0% and about 30% at 2 hours, between about 0% and about 30% at 4 hours, between about 5% and about 70% at 8 hours, between about 10% and about 80% at 12 hours, between about 20% to about 100% at 16 hours, between about 40% and about 100% hours at 24 hours and greater than 50% at 36 hours; or (82) between 0% and about 20% at 1 hour, between about 0% and about 20% at 2 hours, between about 0% and about 20% at 4 hours, between about 0% and about 20% at 8 hours, between about 5% and about 40% at 12 hours, between about 10% to about 80% at 16 hours, between about 40% and about 100% hours at 24 hours and greater than 60% at 36 hours; or (83) between 0% and about 10% at 1 hour, between about 0% and about 20% at 2 hours, between about 0% and about 40% at 4 hours, between about 5% and about 60% at 8 hours, between about 10% and about 80% at 12 hours, between about 20% to about 100% at 16 hours, between about 40% and about 100% hours at 24 hours and greater than 50% at 36 hours; or (84) between 0% and about 50% at 1 hour, between about 0% and about 75% at 2 hours, between about 3% and about 95% at 4 hours, between about 10% and about 100% at 8 hours, between about 20% and about 100% at 12 hours, between about 30% to about 100% at 16 hours, between about 50% and about 100% hours at 24 hours and greater than 80% at 36 hours; or (85) between 0% and about 45% at 1 hour, between about 0% and about 70% at 2 hours, between about 3% and about 90% at 4 hours, between about 8% and about 100% at 8 hours, between about 15% and about 100% at 12 hours, between about 25% to about 100% at 16 hours, between about 45% and about 100% hours at 24 hours and greater than 80% at 36 hours; or (86) between 0% and about 40% at 1 hour, between about 0% and about 65% at 2 hours, between about 0% and about 80% at 4 hours, between about 5% and about 80% at 8 hours, between about 10% and about 90% at 12 hours, between about 20% to about 100% at 16 hours, between about 40% and about 100% hours at 24 hours and greater than 70% at 36 hours; or (87) between 0% and about 35% at 1 hour, between about 0% and about 60% at 2 hours, between about 0% and about 70% at 4 hours, between about 3% and about 70% at 8 hours, between about 5% and about 80% at 12 hours, between about 15% to about 100% at 16 hours, between about 30% and about 100% hours at 24 hours and greater than 40% at 36 hours; or (88) between 0% and about 60% at 1 hour, between about 0% and about 80% at 2 hours, between about 5% and about 100% at 4 hours, between about 15% and about 100% at 8 hours, between about 30% and about 100% at 12 hours, between about 40% to about 100% at 16 hours, between about 60% and about 100% hours at 24 hours and greater than 70% at 36 hours; or (89) between 0% and about 50% at 1 hour, between about 0% and about 75% at 2 hours, between about 5% and about 95% at 4 hours, between about 25% and about 80% at 8 hours, between about 30% and about 100% at 12 hours, between about 40% to about 100% at 16 hours, between about 60% and about 100% hours at 24 hours and greater than 60% at 36 hours; or (90) between 0% and about 60% at 1 hour, between about 0% and about 85% at 2 hours, between about 5% and about 100% at 4 hours, between about 10% and about 100% at 8 hours, between about 20% and about 100% at 12 hours, between about 30% to about 100% at 16 hours, between about 50% and about 100% hours at 24 hours and greater than 80% at 36 hours; or (91) between 15% and about 25% at 1 hour, between about 25% and about 35% at 2 hours, between about 30% and about 45% at 4 hours, between about 40% and about 60% at 8 hours, between about 55% and about 70% at 12 hours and between about 60% to about 75% at 16 hours; or (92) between 10% and about 20% at 1 hour, between about 20% and about 30% at 2 hours, between about 25% and about 40% at 4 hours, between about 30% and about 50% at 8 hours, between about 50% and about 65% at 12 hours and between about 55% to about 65% at 16 hours; or (93) between 5% and about 15% at 1 hour, between about 15% and about 25% at 2 hours, between about 20% and about 35% at 4 hours, between about 25% and about 45% at 8 hours, between about 45% and about 60% at 12 hours and between about 50% to about 60% at 16 hours; or (94) between 15% and about 30% at 1 hour, between about 20% and about 40% at 2 hours, between about 20% and about 50% at 4 hours, between about 30% and about 70% at 8 hours, between about 60% and about 80% at 12 hours and between about 70% to about 90% at 16 hours; or (95) between 0% and about 50% at 1 hour, between about 5% and about 50% at 2 hours, between about 5% and about 70% at 4 hours, between about 10% and about 80% at 8 hours, between about 20% and about 100% at 12 hours and between about 40% to about 100% at 16 hours; or (96) between 15% and about 40% at 1 hour, between about 15% and about 45% at 2 hours, between about 20% and about 60% at 4 hours, between about 20% and about 80% at 8 hours, between about 30% and about 90% at 12 hours and between about 40% to about 100% at 16 hours; or (97) between 0% to about 80% at 0.5 hours, and greater than about 40% at 1 hour; or (98) between 0% to about 40% at 0.5 hours, and greater than about 60% at 1 hour; or (98a) between 0% to about 20% at 0.5 hours, and greater than about 40% at 1 hour; or (99) between 0% to about 20% at 0.5 hours, and greater than about 20% at 1 hour; or (100) between 0% to about 90% at 0.5 hours, and greater than about 60% at 1 hour; or (101) between 0% to about 100% at 0.5 hours, and greater than about 60% at 1 hour; or (102) between 0% to about 90% at 1 hour, and greater than about 40% at 2 hours; or (103) between 0% to about 100% at 1 hour, and greater than about 60% at 2 hours; or (104) between 0% to about 60% at 1 hour, and greater than about 40% at 2 hours; or (105) between 0% to about 40% at 1 hour, and greater than about 30% at 2 hours; or (106) between 0% to about 50% at 1 hour, and greater than about 40% at 2 hours; or (107) between 0% to about 30% at 1 hour, and greater than about 20% at 2 hours; or (108) between 0% and about 50% at 1 hour, between about 0% and about 80% at 2 hours, between about 5% and about 100% at 4 hours and between about 10% and about 100% at 8 hours; or (109) between 10% and about 60% at 1 hour, between about 15% and about 75% at 2 hours, between about 20% and about 95% at 4 hours and between about 30% and about 100% at 8 hours; or (110) between 0% to about 80% at 0.25 hours, and greater than about 90% at 1 hour; or (111) between 0% to about 100% at 0.25 hours, and greater than about 60% at 1 hour. In some preferred embodiments, the foregoing in-vitro release rate of buprenorphine is achieved when measured by the USP Basket or Paddle Method at 100 rpm in 900 mL distilled water (time=0 hour begins here) at 37° C. at any pH between 4.5 and 8 at 37° C., said release rate measured following dissolution (pretreatment) with USP Basket or Paddle Method at 100 rpm in 900 mL of 0.1N HCl for two hours at 37° C., and then following further dissolution (pretreatment) with USP Basket or Paddle Method at 100 rpm in 900 mL distilled water at any pH of between 2 and 4 for a further time of up to two hours at 37° C. In some preferred embodiments, the foregoing dosage forms in (1) to (111) also incorporate a controlled release material and/or provide duodenal delivery, jejunal delivery, ileal delivery, ileo-colonic delivery or colonic delivery. In some preferred embodiments, the foregoing in-vitro release rate of buprenorphine in (1) to (111) are achieved when measured by the USP Basket or Paddle Method at 100 rpm in 900 mL distilled water (time=0 hour begins here) at 37° C. at a pH of between 4.5 and 8 at 37° C., said release rate measured following dissolution (pretreatment) with USP Basket or Paddle Method at 100 rpm in 900 mL of 0.1N HCl for about 1, 1.5 or 2 hours at 37° C. In some preferred embodiments, the foregoing in-vitro release rate of buprenorphine in (1) to (111) are achieved when measured by the USP Basket or Paddle Method at 100 rpm in 900 mL distilled water (time=0 hour begins here) at 37° C. at a pH of between 4.5 and 8 at 37° C., said release rate measured following dissolution (pretreatment) with USP Basket or Paddle Method at 100 rpm in 900 mL of 0.1N HCl for about 1, 1.5 or 2 hours at 37° C., and then following further dissolution (pretreatment) with USP Basket or Paddle Method at 100 rpm in 900 mL distilled water at a pH of between 2 and 4 for a further time of up to two hours at 37° C. In some preferred embodiments, the foregoing in-vitro release rate of buprenorphine in (1) to (111) are achieved when measured by the USP Basket or Paddle Method at 100 rpm in 900 mL distilled water at 37° C. at a pH of between 4.5 and 8 at 37° C.

The term “optionally a controlled release material” means that the dosage may or may not be require one or more controlled release material to achieve some, most, substantially all or all of the objectives, specifications or claims of the invention.

In some preferred embodiments, the dosage form provides an oral pharmaceutical composition for the treatment of a buprenorphine responsive medical condition comprising a therapeutically effective amount of buprenorphine or pharmaceutically acceptable salts thereof or mixture thereof, and optionally a controlled release material, said buprenorphine given alone or in combination with another drug in the same dosage form or in a different dosage form to treat the same or a different condition or to treat side effects of buprenorphine or to deter abuse of the buprenorphine.

In some preferred embodiments, the dosage form provides a pharmaceutical dosage form for the treatment of a buprenorphine responsive medical condition comprising a therapeutically effective amount of buprenorphine or pharmaceutically acceptable salts thereof or mixture thereof, said dosage form resistant or substantially resistant to dissolution and/or absorption in the stomach, and/or in the duodenum, and/or in the jejunum, and/or in the ileum, or in the small intestine, or in the stomach and duodenum, or in the stomach, duodenum and jejunum, or in the stomach, duodenum, jejunum and terminal ileum, or in the stomach and small intestine, or before it reaches the ileo-cecal junction, or until it crosses the ileo-cecal junction, or until it reaches the colon; said buprenorphine in the dosage form released rapidly or slowly upon reaching a the desired anatomic region of the GI tract (e.g., ileum or colon) or upon reaching the desired gastrointestinal conditions conducive to release from the dosage form (e.g., osmotic pressure, pH, time after ingestion, microbial flora); said dosage form in some embodiments providing immediate release of buprenorphine following the expected lag time; said dosage form in some other embodiments providing sustained release of buprenorphine following the expected lag time.

In some embodiments, the oral buprenorphine dosage form is substantially non-releasable in the stomach and small intestine, or substantially non-releasable in the stomach and duodenum, or substantially non-releasable in the stomach, duodenum and jejunum, or substantially non-releasable in the stomach, duodenum, jejunum and terminal ileum, or substantially non-releasable until it reaches the ileum, or substantially non-releasable until it reaches the colon.

In some embodiments, the oral buprenorphine dosage form is substantially non-releasable until up to about 1, or 1.5, 2, or 2.25, or 2.5, or 2.75, or 3, or 3.25, or 3.5, or 3.75, or 4, or 4.25, or 4.5, or 4.75, or 5, or 5.25, or 5.5, or 5.75, or 6, or 6.25, or 6.5, or 7.75, or 7, or 7.25, or 7.5, or 7.75, or 8, or 8.25, or 8.5, or 8.75, or 9, or 9.25, or 9.5, or 9.75, or 10, or 10.25, or 10.5, or 10.75, or 11, or 11.25, or 1.5, or 11.75, or 12, or 14, or 16, or 18, or 20 hours after oral ingestion of the oral dosage form. In some particularly preferred embodiments, said dosage form is substantially non-releasable until up to about 2.5, or 2.75, or 3, or 3.25, or 3.5, or 3.75, or 4, or 4.25, or 4.5, or 4.75, or 5, or 5.25, or 5.5, or 5.75, or 6, or 6.25, or 6.5, or 7.75, or 7 hours after oral ingestion of the oral dosage form.

In some preferred embodiments, the oral buprenorphine dosage form includes a coated capsule or tablet wherein the coating comprises material which dissolves at a pH≧5, or ≧5.5, or ≧5.7, or ≧6, or ≧6.2, or ≧6.4, or ≧6.6, or ≧6.8, ≧7, or ≧7.2. In some other preferred embodiments, the oral buprenorphine dosage form includes material incorporated in dosage form, wherein the material substantially resists dissolution for at least about 1, or 1.5, or 2, or 2.5, or 3, or 3.25, or 3.5, or 3.75, or 4, or 4.25, or 4.5, or 4.75, or 5, or 5.25, or 5.5, or 5.75, or 6, or 6.25, or 6.5, or 7.75, or 7 31 hours at a pH of about ≦5, or ≦5.5, or ≦5.7, or ≦6, or ≦6.2, or ≦6.4, or ≦6.6, or ≦6.8, ≦7, or ≦7.2.

In some embodiments, the oral buprenorphine dosage form is coated with or includes incorporated one or more of the following: (i) cellulose acetate trimellitiate (CAT); (ii) hydroxypropylmethyl cellulose phthalate (HPMCP); (iii) polyvinyl acetate phthalate (PVAP); (iv) shellac; (v) a copolymer of methacrylic acid and methylmethacrylate; (vi) a material which is redox-sensitive; (vii) an azopolymer or a disulphide polymer; (viii) a material which is degraded by enzymes or bacteria present in the colon; (ix) a copolymer of methacrylic acid and methylmethacrylate to which has been added during polymerization the monomer methyl acrylate; (x) a cellulose ester; (xi) polyvinyl acetate phthalate.

In some embodiments, the dosage form consists of a coated capsule wherein the coating is applied separately to empty capsule body and cap. In some embodiments, the dosage form consists of a coated capsule filled with a caplet or tablet.

In some embodiments, the dosage form is coated with a film or incorporates material which makes the dosage form: (i) is non-dissolving at pH <3 to 4 and dissolving at pH>5; or (ii) non-dissolving at pH <3 to 4 and dissolving at pH>5.5; or (iii) non-dissolving at pH <3 to 4 and dissolving at pH>6; or (iv) non-dissolving at pH <3 to 4.5 and dissolving at pH>6; or (v) non-dissolving at pH <3 to 4 and dissolving at pH>6.5; or (vi) non-dissolving at pH <3 to 4.5, and dissolving at pH>6.5; or (vii) non-dissolving at pH <3 to 4 and dissolving at pH>7; or (viii) non-dissolving at pH <3 to 4.5 and dissolving at pH>7; or (ix) is non-dissolving at pH <3 to 5 and dissolving at pH>7; or (x) non-dissolving at pH <3 to 5.5 and dissolving at pH>7.

In some embodiments, the oral buprenorphine dosage form is non-dissolving or substantially non-dissolving at pH <5.5, or at pH <6.0, or at pH <6.2, or at pH <6.5, or at pH <6.8, or at pH <7.0, when measured by USP Basket Method or USP Paddle Method at 100 rpm in 900 mL of water at 37° C. (adjusted to the required pH with hydrochloric acid or sodium hydroxide) for up to about 2, 2.5, 3, 3.5, 4, 4.5, or 5.

In some embodiments, the oral buprenorphine dosage form is non-releasing or substantially non-releasing at pH <5.5, or at pH <6.0, or at pH <6.2, or at pH <6.5, or at pH <6.8, or at pH <7.0, when measured by USP Basket Method or USP Paddle Method at 100 rpm in 900 mL of water at 37° C. (adjusted to the required pH with hydrochloric acid or sodium hydroxide) for up to about 2, 2.5, 3, 3.5, 4, 4.5, or 5.

In some embodiments, the oral buprenorphine dosage form is non-releasing or substantially non-releasing for up to about 2, 2.5, 3, 3.5, 4, 4.5, or 5 hours following ingestion.

In some embodiments, the oral buprenorphine dosage form comprises material which is non-dissolving or substantially non-dissolving at a particular pH or range of pH and is dissolving or substantially dissolving at another pH or another range of pH, said material commingled with the buprenorphine API or with the granulation containing buprenorphine API.

In some embodiments, the oral buprenorphine dosage form comprises material which is non-dissolving or substantially non-dissolving at a particular pH or range of pH and is dissolving or substantially dissolving at another pH or another range of pH, said material commingled with the buprenorphine API or with the granulation containing buprenorphine API, in addition to being coated on the dosage form.

In some embodiments, the specifications regarding coating of the dosage form of the invention with controlled release material or pH sensitive material are also applicable to dosage forms where said material is commingled with the buprenorphine API or with the granulation containing buprenorphine API, instead of or in addition to coating the dosage form.

In some embodiments, the specifications regarding coating of the dosage form of the invention with controlled release material or pH sensitive material are also applicable to dosage forms where is the coating is applied to multiparticulate matrices or to subunits of the dosage form e.g., beads incorporating drug), instead of or in addition to coating the dosage form.

In some embodiments, the dosage form consists of a coated capsule wherein the coating is applied to capsules having a sealing on the gap between capsule body and cap.

In some embodiments, the dosage form consists of a coated capsule containing a buprenorphine, wherein the capsule is coated with a material selected from the group comprising cellulose acetate trimellitiate, hydroxypropylmethyl cellulose phthalate, polyvinyl acetate phthalate, shellac, and a copolymer of methacrylic acid and ethyl acrylate, azopolymers, disulphide polymers and amylose.

In some preferred embodiments, the oral buprenorphine dosage form has a buprenorphine Tmax that exceeds its dosing frequency.

In some preferred embodiments, the buprenorphine Tmax ratio of the oral buprenorphine dosage form of the invention to buprenorphine given orally as a conventional solution, suspension, immediate release tablet or capsule or given sublingually or bucally is ≧1.25, or ≧1.5, or ≧1.75, or ≧2, or ≧2.5, or ≧3, or ≧3.5, or ≧4, or ≧4.5, or ≧5, or ≧5.5, or ≧6, or ≧6.5, or ≧7, or ≧7.5, or ≧8, or ≧8.5, or ≧9, or ≧9.5, or ≧10, or ≧10.5, or ≧12, or ≧14, or ≧16, or ≧18, or ≧20.

In some preferred embodiments, the norbuprenorphine Tₘₐₓ ratio of the oral buprenorphine dosage form of the invention to buprenorphine given orally as a conventional solution, suspension, immediate release tablet or capsule or given sublingually or bucally is ≧1.25, or ≧1.5, or ≧1.75, or ≧2, or ≧2.5, or ≧3, or ≧3.5, or ≧4, or ≧4.5, or ≧5, or ≧5.5, or ≧6, or ≧6.5, or ≧7, or ≧7.5, or ≧8, or ≧8.5, or ≧9, or ≧9.5, or ≧10, or ≧10.5, or ≧12, or ≧14, or ≧16, or ≧18, or ≧20.

In some preferred embodiments, the buprenorphine Cmax ratio after buprenorphine given orally as a conventional solution, suspension, immediate release tablet or capsule, or given sublingually or bucally, to the oral buprenorphine dosage form of the invention given orally is ≧1.1, or ≧1.2, or ≧1.3, or ≧1.5, or ≧1.5, or ≧1.6, or ≧1.7, or ≧1.8, or ≧1.9, or ≧2, or ≧2.2, or ≧2.5, or ≧3, or ≧3.5, or ≧4, or ≧4.5, or ≧5, or ≧5.5, or ≧6, or ≧6.5, or >7, or ≧7.5, or ≧8, or ≧8.5, or ≧9, or ≧9.5, or ≧10, or ≧10.5, or ≧12, or ≧14, or ≧16, or ≧18, or ≧20.

In some preferred embodiments, the norbuprenorphine Cmax ratio of buprenorphine given orally as a conventional solution, suspension, immediate release tablet or capsule, or given sublingually or bucally, to the oral buprenorphine dosage form of the invention given orally is ≧1.1, or ≧1.2, or ≧1.3, or ≧1.5, or ≧1.5, or ≧1.6, or ≧1.7, or ≧1.8, or ≧1.9, or ≧2, or ≧2.2, or ≧2.5, or ≧3, or ≧3.5, or ≧4, or ≧4.5, or ≧5, or ≧5.5, or ≧6, or ≧6.5, or ≧7, or ≧7.5, or ≧8, or ≧8.5, or ≧9, or ≧9.5, or ≧10, or ≧10.5, or ≧12, or ≧14, or ≧16, or ≧18, or ≧20.

In some preferred embodiments, the buprenorphine AUC₀₋₂₄ ratio of the oral buprenorphine dosage form of the invention to buprenorphine given orally as a conventional solution, suspension, immediate release tablet or capsule is ≧1.1, or ≧1.2, or ≧1.3, or ≧1.5, or ≧1.5, or ≧1.6, or ≧1.7, or ≧1.8, or ≧1.9, or ≧2, or ≧2.2, or ≧2.5, or ≧3.

In some preferred embodiments, the buprenorphine AUC_{0-inf} ratio of the oral buprenorphine dosage form of the invention to buprenorphine given orally as a conventional solution, suspension, immediate release tablet or capsule is ≧1.1, or ≧1.2, or ≧1.3, or ≧1.5, or ≧1.5, or ≧1.6, or ≧1.7, or ≧1.8, or ≧1.9, or ≧2, or ≧2.2, or ≧2.5, or ≧3.

In some preferred embodiments, the norbuprenorphine AUC₀₋₂₄ ratio of the oral buprenorphine dosage form of the invention to buprenorphine given orally as a conventional solution, suspension, immediate release tablet or capsule is ≧1.1, or ≧1.2, or ≧1.3, or ≧1.5, or ≧1.5, or ≧1.6, or ≧1.7, or ≧1.8, or ≧1.9, or ≧2, or ≧2.2, or ≧2.5, or ≧3.

In some preferred embodiments, the norbuprenorphine AUC_{0-inf} ratio of the oral buprenorphine dosage form of the invention to buprenorphine given orally as a conventional solution, suspension, immediate release tablet or capsule is ≧1.1, or ≧1.2, or ≧1.3, or ≧1.5, or ≧1.5, or ≧1.6, or ≧1.7, or ≧1.8, or ≧1.9, or ≧2, or ≧2.2, or ≧2.5, or ≧3.

In some preferred embodiments, the apparent oral clearance ratio of buprenorphine given orally as a conventional solution, suspension, immediate release tablet or capsule, to the oral buprenorphine dosage form of the invention given orally is ≧1.1, or ≧1.2, or ≧1.3, or ≧1.4, or ≧1.5.

In some embodiments, the oral modified release buprenorphine dosage form releases less than about 0.1%, or 0.5%, or 1%, or 1.5%, or 2%, or 2.5%, or 3%, or 3.5%, or 4%, or 4.5%, or 5%, or 6%, or 7%, or 8%, or 9%, or 10%, or 12%, or 14%, or 15%, or 16%, or 17%, or 18%, or 20% of buprenorphine in vitro from the dosage form when measured at about 1, 1.5, 2, 2.5, 3, 3.25, 3.5, 3.75, 4, 4.25, 4.5, 4.75, 5, 5.5, 6, 6.5, 7, 7.5, 8, 8.5, 9, 9.5, 10, 10.5, 10.5, 11, 11.5, or 12 hours, said in vitro release measured by the USP Basket or Paddle Method at 100 rpm in 100 to 900 mL in one or more of the following: (a) water at 37° C. at a pH of 4.5, adjusted with HCl; (b) water at 37° C. at a pH of 5, adjusted with HCl; (c) water at 37° C. at a pH of 5.5, adjusted with HCl; (d) simulated gastric fluid at 37° C.; (e) simulated intestinal fluid at 37° C.; (f) simulated gastric fluid at 37° C. for one hour followed by a switch to simulated intestinal fluid; (g) Phosphate buffer 0.067M (pH 7.0) at 37° C.; and (h) Phosphate buffer 0.067M (pH 7.0) containing Tween 80 at 37° C. In some embodiments, said in vitro dissolution is measured by the USP Apparatus III (Reciprocating Cylinder) Method instead of the Basket or Paddle Method. In some embodiments, most preferably, the dosage form releases less than about 0.1%, or 0.5%, or 1%, or 1.5%, or 2%, or 2.5%, or 3%, or 3.5%, or 4%, or 4.5%, or 5%, or 6%, or 7%, or 8%, or 9%, or 10 of buprenorphine in vitro from the dosage form when measured at about 1.5, 2, 2.5, 3, 3.5, 4, 4.5, 5, 5.5, 6, 6.5, or 7 hours.

In some embodiments, the oral modified release buprenorphine dosage form releases 0%, or less than about 0.1%, or 0.5%, or 1%, or 1.5%, or 2%, or 2.5%, or 3%, or 3.5%, or 4%, or 4.5%, or 5%, or 6%, or 7%, or 8%, or 9%, or 10%, or 12%, or 14%, or 15%, or 16%, or 17%, or 18%, or 20% or 25% or 28% or 30% or 35% of buprenorphine in vivo from the dosage form prior to reaching the duodenum, or jejunum, or ileum, or terminal ileum, or ileo-cecal junction, or ascending colon, or transverse colon, or descending colon, or colon

In some embodiments, the oral modified release buprenorphine dosage form releases 0%, or less than about 0.1%, or 0.5%, or 1%, or 1.5%, or 2%, or 2.5%, or 3%, or 3.5%, or 4%, or 4.5%, or 5%, or 6%, or 7%, or 8%, or 9%, or 10%, or 12%, or 14%, or 15%, or 16%, or 17%, or 18%, or 20% or 25% or 28% or 30% or 35% of buprenorphine in vivo from the dosage form for at least about 1 hour, or at least about 1.5 hours, or at least about 2 hours, or at least about 2.5 hours, or at least about 3 hours, or at least about 3.25 hours, or at least about 3.5 hours, or at least about 3.75 hours, or at least about 4 hours, or at least about 4.25 hours, or at least about 4.5 hours, or at least about 4.75 hours, or at least about 5 hours, or at least about 5.25 hours, or at least about 5.5 hours, or at least about 5.75 hours, or at least about 6 hours, or at least about 6.25 hours, or at least about 6.5 hours, or at least about 6.75 hours, or at least about 7 hours, or at least about 7.25 hours, or at least about 7.5 hours, or at least about 7.75 hours, or at least about 8 hours, or at least about 8.25 hours, or at least about 8.5 hours, or at least about 8.75 hours, or at least about 9 hours, or at least about 9.25 hours, or at least about 9.5 hours, or at least about 9.75 hours, or at least about 10 hours, or at least about 10.25 hours, or at least about 10.5 hours, or at least about 10.75 hours, or at least about 11 hours, or at least about 11.5 hours, or at least about 12 hours after oral ingestion, said in vivo release from the dosage form measured by appearance of buprenorphine in plasma, using AUC₀₋ₙ/AUC_{0-inf}, or AUC₀₋ₙ/AUC_{0-τ}, where “n” is the time after oral ingestion. Most preferably, the time after oral ingestion is at least about 2 hours, or at least about 2.5 hours, or at least about 3 hours, or at least about 3.5 hours, or at least about 4 hours, or at least about 4.5 hours, or at least about 5 hours, or at least about 5.5 hours, or at least about 6 hours, or at least about 6.5 hours, or at least about 7 hours.

In some embodiments, the oral modified release buprenorphine dosage form releases 0%, or less than about 0.1%, or 0.5%, or 1%, or 1.5%, or 2%, or 2.5%, or 3%, or 3.5%, or 4%, or 4.5%, or 5%, or 6%, or 7%, or 8%, or 9%, or 10%, or 12%, or 14%, or 15%, or 16%, or 17%, or 18%, or 20% or 25% or 28% or 30% or 35% of buprenorphine in vivo from the dosage form when the average measured or expected gastrointestinal pH is less than about 3.5, or is less than about 4, or is less than about 4.5, or is less than about 5, or less than about 5.2, or less than about 5.4, or less than about 5.6, or less than about 5.8, or less than about 6, or less than about 6.2, or less than about 6.4, or less than about 6.5, or less than about 6.6, or less than about 6.7, or less than about 6.8, or less than about 6.9, or less than about 7, or less than about 7.1, or less than about 7.2, or less than about 7.3, or less than about 7.4, or less than about 7.5, or less than about 7.6, or less than about 7.7, or less than about 7.8, or less than about 7.9, or less than about 8, or less than about 8.1, or less than about 8.2, or less than about 8.3, or less than about 8.4, or less than about 8.5, or less than about 8.6, or less than about 8.7, or less than about 8.8, or less than about 9, when measured up to about 1 hour, or up to about 1.5 hours, or up to about 2 hours, or up to about 2.5 hours, or up to about 2.75 hours, or up to about 3 hours, or up to about 3.25 hours, or up to about 3.5 hours, or up to about 3.75 hours, or up to about 4 hours, or up to about 4.25 hours, or up to about 4.5 hours, or up to about 4.75 hours, or up to about 5 hours, or up to about 5.25 hours, or up to about 5.5 hour, or up to about 5.75 hours, or up to about 6 hours, or up to about 6.5 hours, or up to about 6.75 hours, or up to about 7 hours, or up to about 7.25 hours, or up to about 7.5 hours, or up to about 7.5 hours, or up to about 7.75 hours, or up to about 8 hours, or up to about 8.25 hours, or up to about 8.5 hours, or up to about 8.75 hours, or up to about 9 hours, or up to about 9.25 hours, or up to about 9.5 hours, or up to about 9.75 hours, or up to about 10 hours, or up to about 10.5 hours, or up to about 11 hours, or up to about 12 hours, or up to about 12.5 hours after oral ingestion. Most preferably, the dosage form releases 0%, or less than about 0.1%, or 0.5%, or 1%, or 1.5%, or 2%, or 2.5%, or 3%, or 3.5%, or 4%, or 4.5%, or 5%, or 6%, or 7%, or 8%, or 9%, or 10%, or 12%, or 14%, or 15%, or 16%, or 17%, or 18%, or 20% or 25% or 28% or 30% or 35% of buprenorphine in vivo when the measured or expected gastrointestinal pH is less than about 5, or less than about 5.5, or less than about 6, or less than about 6.5, or less than about 6.8, or less than about 7, or less than about 7.2, or less than about 7.5. Most preferably, said release is measured at about 2 hours, or about 2.5 hours, or about 3 hours, or 4 hours or 4.5 hours or 5 hours, or 5.5 hours or 6 hours or 6.5 hours or 7 hours.

In some embodiments, the targeted gastrointestinal delivery of the buprenorphine from the oral modified release buprenorphine dosage form into the lower segments of the gastrointestinal tract can be achieved through a variety of approaches, including but limited to incorporation of material or processes to achieve one or more of the following: time-controlled, pH-controlled, pressure-controlled, enzyme-controlled and hydration-controlled. Since the gastrointestinal tract is a complex, variable and highly dynamic environment and further complicated by the volume, content and location of food and beverages, in some embodiments, incorporation of material to achieve more than one of the above approaches is preferred.

In some embodiments, the targeted gastrointestinal delivery of the buprenorphine from the oral modified release buprenorphine dosage form into the lower segments of the gastrointestinal tract can be achieved through encapsulation of the buprenorphine, preferably with excipients or functional excipients, said capsule incorporating, coated with or overcoated with material or processes to achieve targeted gastrointestinal delivery.

In some embodiments, the oral modified release buprenorphine dosage form is coated with a material or incorporates material which is non-dissolving or substantially resistant to dissolution, each when measured by USP Basket Method or USP Paddle Method at 100 rpm in 900 mL of water at 37° C. (adjusted to the required pH with hydrochloric acid or sodium hydroxide) at about pH 2, pH 2.2, pH 2.4, pH 2.6, pH 2.8, pH 3, pH 3.2, pH 3.4, pH 3.6, pH 3.8, pH 4, pH 4.2, pH 4.4, pH 4.6, pH 4.8, pH 5, pH 5.2, pH 5.4, pH 5.6, pH 5.8, pH 6, pH 6.2, pH 6.4, pH 6.6, pH 6.8, pH 7, pH 7.2, pH 7.4, for up to about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 14, 16, 18, or 20 hours.

In some embodiments, the oral modified release buprenorphine dosage form is coated with a material or incorporates material which is non-releasing or substantially non-releasing, each when measured by USP Basket Method or USP Paddle Method at 100 rpm in 900 mL of water at 37° C. (adjusted to the required pH with hydrochloric acid or sodium hydroxide) at about pH 2, pH 2.2, pH 2.4, pH 2.6, pH 2.8, pH 3, pH 3.2, pH 3.4, pH 3.6, pH 3.8, pH 4, pH 4.2, pH 4.4, pH 4.6, pH 4.8, pH 5, pH 5.2, pH 5.4, pH 5.6, pH 5.8, pH 6, pH 6.2, pH 6.4, pH 6.6, pH 6.8, pH 7, pH 7.2, pH 7.4, for up to about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 14, 16, 18, or 20 hours.

In some embodiments, the oral modified release buprenorphine dosage form is coated with a material or incorporates material which is non-dissolving or substantially non-dissolving at one pH but dissolving or substantially dissolving at another pH for up to about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 14, 16, 18, or 20 hours; said one pH being≦2, or ≦2.1, or ≦2.2, or ≦2.3, or ≦2.4, or ≦2.5, or ≦2.6, or ≦2.7, or ≦2.8, or ≦2.9, or ≦3, or ≦3.1, or ≦3.2, or ≦3.3, or ≦3.4, or ≦3.5, or ≦3.6, or ≦3.7, or ≦3.8, or ≦3.9, or ≦4, or ≦4.1, or ≦4.2, or ≦4.3, or ≦4.4, or ≦4.5, or ≦4.6, or ≦4.7, or ≦4.8, or ≦4.9, or ≦5; said another pH being ≧5.2, or ≧5.3, or ≧5.4, or ≧5.5, or ≧5.6, or ≧5.7, or ≧5.8, or ≧5.9, or ≧6, or ≧6.1, or ≧6.2, or ≧6.3, or ≧6.4, or ≧6.5, or ≧6.6, or ≧6.7, or ≧6.8, or ≧6.9, or ≧7, or ≧7.1, or ≧7.2, or ≧7.3, or ≧7.4, or ≧7.5, or ≧7.6, or ≧7.7, or ≧7.8, or ≧7.9, or ≧8.0, or ≧8.1, or ≧8.2, or ≧8.3, or ≧8.4, or ≧8.5, or ≧8.6, or ≧8.7, or ≧8.8, or ≧8.9, or ≧9.0, each when measured when by USP Basket Method or USP Paddle Method at 100 rpm in 900 mL of water at 37° C. (adjusted to the required pH with hydrochloric acid or sodium hydroxide).

In some embodiments, the oral modified release buprenorphine dosage form is coated with a material or incorporates material which is non-releasing or substantially non-releasing at one pH but releasing or substantially releasing at another pH for up to about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 14, 16, 18, or hours; said one pH being≦2, or ≦2.1, or ≦2.2, or ≦2.3, or ≦2.4, or ≦2.5, or ≦2.6, or ≦2.7, or ≦2.8, or ≦2.9, or ≦3, or ≦3.1, or ≦3.2, or ≦3.3, or ≦3.4, or ≦3.5, or ≦3.6, or ≦3.7, or ≦3.8, or ≦3.9, or ≦4, or ≦4.1, or ≦4.2, or ≦4.3, or ≦4.4, or ≦4.5, or ≦4.6, or ≦4.7, or ≦4.8, or ≦4.9, or ≦5; said another pH being ≧5.2, or ≧5.3, or ≧5.4, or ≧5.5, or ≧5.6, or ≧5.7, or ≧5.8, or ≧5.9, or ≧6, or ≧6.1, or ≧6.2, or ≧6.3, or ≧6.4, or ≧6.5, or ≧6.6, or ≧6.7, or ≧6.8, or ≧6.9, or ≧7, or ≧7.1, or ≧7.2, or ≧7.3, or ≧7.4, or ≧7.5, or ≧7.6, or ≧7.7, or ≧7.8, or ≧7.9, or ≧8.0, or ≧8.1, or ≧8.2, or ≧8.3, or ≧8.4, or ≧8.5, or ≧8.6, or ≧8.7, or ≧8.8, or ≧8.9, or ≧9.0, each when measured when by USP Basket Method or USP Paddle Method at 100 rpm in 900 mL of water at 37° C. (adjusted to the required pH with hydrochloric acid or sodium hydroxide).

In some embodiments, the oral modified release buprenorphine dosage form is non-bioavailable or substantially non-bioavailable for up to about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 14, 16, 18, or 20 hours after oral ingestion (e.g., the dosage form releases 0%, or less than about 0.1%, or 0.5%, or 1%, or 1.5%, or 2%, or 2.5%, or 3%, or 3.5%, or 4%, or 4.5%, or 5%, or 6%, or 7%, or 8%, or 9%, or 10%, or 12%, or 14%, or 15%, or 16%, or 17%, or 18%, or 20% or 25% or 28% or 30% or 35% of buprenorphine in vivo when assessed up to the specified time). In some embodiments, the oral modified release buprenorphine dosage form is coated with a material or incorporates material which renders the dosage form non-bioavailable or substantially non-bioavailable for up to about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 14, 16, 18, or 20 hours. Most preferably, said dosage form is non-bioavailable or substantially non-bioavailable for up to about 2 hours, or about 2.5 hours, or about 3 hours, or 4 hours or 4.5 hours or 5 hours, or 5.5 hours or 6 hours or 6.5 hours or 7 hours.

It is an object of certain preferred embodiments of the present invention to provide formulations which provide a substantially improved dose proportional extent of absorption compared to immediate release formulations.

It is an object of certain preferred embodiments of the present invention to provide formulations which provide a substantially reduced euphoric, psychic or pleasurable effect compared to immediate release formulations.

It is an object of certain preferred embodiments of the present invention to provide bioavailable formulations for oral administration suitable for up to once-a-day administration or up to once every 2 or every 3 days.

It is an object of certain preferred embodiments of the present invention to provide formulations for oral administration suitable for up to once-a-day administration which provide a delayed onset and sustained duration of therapeutic effect.

It is an object of certain preferred embodiments of the invention to provide an oral formulation which provides a sustained duration of therapeutic effect.

It is an object of certain preferred embodiments of the present invention to provide oral formulations which provide therapeutic effects for up to about 30 minutes. In other preferred embodiments, the oral formulations provide therapeutic effects for up to about 1 hour, or up to about 2 hours, or up to about 4 hours, or up to about 6 hours, or up to about 8 hours, or up to about 10 hours, or up to about 12 hours, or up to about 16 hours, or up to about 18 hours, or up to about 24 hours, or up to about every 30 hours, or up to about every 36 hours, or up to about 48 hours, or up to about 60 hours, or up to about 72 hours, or up to about 96 hours, or up to about every 168 hours.

Some or all of the above objects and others are achieved by embodiments of the present invention, which is directed in part to a dosage form of orally administered buprenorphine.

In some preferred embodiments, the invention comprises an oral pharmaceutical composition for the treatment of diseases and disorders comprising a therapeutically effective amount of a buprenorphine or a pharmaceutically acceptable salt thereof or a mixture thereof.

It is an object of certain embodiments of the present invention to provide oral pharmaceutical compositions of buprenorphine for the treatment of diseases and disorders, said composition in modified release form.

It is an object of certain embodiments of the present invention to provide oral pharmaceutical compositions of buprenorphine for the treatment of diseases and disorders, said composition in sustained release form.

It is an object of certain embodiments of the present invention to provide oral pharmaceutical compositions of buprenorphine for the treatment of diseases and disorders, said composition in controlled release form.

It is an object of certain embodiments of the present invention to provide oral pharmaceutical compositions of buprenorphine for the treatment of diseases and disorders, said composition in extended release form.

It is an object of certain embodiments of the present invention to provide oral pharmaceutical compositions of buprenorphine for the treatment of diseases and disorders, said compositions in modified release form, said dosage form releasing 0%, or less than about 0.1%, or 0.5%, or 1%, or 1.5%, or 2%, or 2.5%, or 3%, or 3.5%, or 4%, or 4.5%, or 5%, or 6%, or 7%, or 8%, or 9%, or 10%, or 12%, or 14%, or 15%, or 16%, or 17%, or 18%, or 20% or 25% or 28% or 30% or 35% of buprenorphine in vivo from the dosage form prior to reaching the duodenum, or jejunum, or ileum, or terminal ileum, or ileo-cecal junction, or ascending colon, or transverse colon, or descending colon, or colon.

It is an object of certain embodiments of the present invention to provide oral pharmaceutical compositions of buprenorphine for the treatment of diseases and disorders, said compositions in modified release form, said dosage form releasing most, substantially all or all of the releasable buprenorphine in the lower segment of the gastrointestinal tract (e.g., distal to the duodenum, or jejunum, or ileum, or terminal ileum, or ileo-cecal junction, ascending colon, or transverse colon), said release occurring over about 4, 5, 6, 7, 8, 9, 10, 12, 14, 15, 16, 17, 18, 19, 20, 21, 22, 23, 24, 25, 26, 30, 32, 36 or 40 hours.

It is an object of certain embodiments of the present invention to provide oral pharmaceutical compositions of buprenorphine for the treatment of diseases and disorders, said compositions in modified release form, said dosage form releasing most, substantially all or all of the releasable buprenorphine in the lower segment of the gastrointestinal tract (e.g., distal to the duodenum, or jejunum, or ileum, or terminal ileum, or ileo-cecal junction, ascending colon, or transverse colon), said release occurring over about 0.25, 0.5, 0.75, 1, 1.5, 2, 2.5, 3, 3.5 or 4 hours.

It is an object of certain embodiments of the present invention to provide oral pharmaceutical compositions of buprenorphine for the treatment of diseases and disorders, said composition in modified, controlled, sustained or extended release form, said dosage form releasing 0%, or less than about 0.1%, or 0.5%, or 1%, or 1.5%, or 2%, or 2.5%, or 3%, or 3.5%, or 4%, or 4.5%, or 5%, or 6%, or 7%, or 8%, or 9%, or 10%, or 12%, or 14%, or 15%, or 16%, or 17%, or 18%, or 20% or 25% or 28% or 30% or 35% of buprenorphine proximal to the duodenum, or jejunum, or ileum, or terminal ileum, or ileo-cecal junction.

It is an object of certain embodiments of the present invention to provide oral pharmaceutical compositions of buprenorphine for the treatment of diseases and disorders, said composition in modified, controlled, sustained or extended release form, or for delivery distal to the duodenum, or jejunum, or ileum, or terminal ileum, or ileo-cecal junction, said dosage form containing up to about 50%, or up to about 45%, or up to about 40%, or up to about 35%, or up to about 30%, or up to about 25%, or up to about 20%, or up to about 15%, or up to about 10%, or up to about 5% of the buprenorphine dose in immediate release form, said immediate release form released and/or available for absorption in the stomach, duodenum, jejunum and/or ileum, and said immediate release form released and/or available for absorption at a pH of less than about 1, or less than about 1.5, or less than about 2, or less than about 2.5, or less than about 3, or less than about 3.5, or less than about 4, or less than about 4.5, or less than about 5, or less than about 5.5, or less than about 6, or less than about 7.

It is an object of certain preferred embodiments of the present invention to provide oral extended release pharmaceutical compositions of buprenorphine that have greater bioavailability (AUC) than oral immediate release formulations.

It is an object of certain preferred embodiments of the present invention to provide oral immediate release and oral extended release pharmaceutical compositions of buprenorphine that provide a greater plasma AUC ratio of norbuprenorphine (an active metabolite of buprenorphine) to buprenorphine than is attained by sublingually administered dosage forms.

It is an object of certain preferred embodiments of the present invention to provide oral immediate release and oral extended release pharmaceutical compositions of buprenorphine that provide a greater plasma AUC ratio of norbuprenorphine to buprenorphine than is attained by transdermal dosage forms.

It is an object of certain preferred embodiments of the present invention to provide oral immediate release and oral extended release pharmaceutical compositions of buprenorphine that provide a greater plasma AUC ratio of norbuprenorphine to buprenorphine than is attained by intranasally administered dosage forms.

It is an object of certain preferred embodiments of the present invention to substantially improve the efficiency and quality of pain management in human patients experiencing moderate or severe pain.

It is an object of certain preferred embodiments of the present invention to substantially improve the efficiency and quality of pain management in human patients experiencing mild to moderate pain.

It is an object of certain preferred embodiments of the present invention to substantially improve the efficiency and quality of pain management in human patients experiencing moderate or severe pain.

It is an object of certain preferred embodiments of the present invention to provide bioavailable oral buprenorphine formulations suitable for up to once-daily administration which substantially improve the efficiency and quality of pain management.

It is an object of some embodiments of the invention to provide oral pharmaceutical compositions of buprenorphine and methods for the treatment of pain, addiction disorders and other buprenorphine responsive disorders, wherein the dosage form is administered at a prespecified dosing regimen. In some embodiments, said dosing regimen associated with reduced side effects, improved tolerability, improved efficiency of therapeutic response, reduced breakthrough symptoms (e.g., breakthrough pain) and reduced treatment discontinuation due to side effects.

It is an object of certain preferred embodiments of the present invention to substantially improve the efficiency and quality of addiction disorder management, particularly opioid addiction disorders and poly-substance abuse disorders in human patients.

It is an object of certain preferred embodiments of the present invention to treat pain and addiction disorders in patients who have a high risk of opioid abuse, continued opioid abuse and relapse from an illicit opioid free state.

It is an object of certain preferred embodiments of the present invention to treat pain and addiction disorders in patients who have a suboptimal efficacy or safety response to sublingual buprenorphine.

It is an object of certain preferred embodiments of the present invention to treat pain and addiction disorders in patients who have a suboptimal efficacy or safety response to transdermal buprenorphine.

It is an object of certain preferred embodiments of the present invention to treat pain and addiction disorders in patients who require a prolonged duration of effect that is provided by sublingual buprenorphine.

It is an object of certain preferred embodiments of the present invention to treat addiction disorders in patients who have a suboptimal efficacy or safety response to methadone.

It is an object of certain preferred embodiments of the present invention to treat pain in patients who have a suboptimal efficacy or safety response with other orally approved opioids (e.g., opioids described for oral administration in the FDA's Orange Book. Goodman & Gilman's The Pharmacological Basis of Therapeutics (Brunton, Lazo and Parker, eds, 11th ed., McGraw Hill (2005); Principles of Analgesic Use in the Treatment of Acute Pain and Cancer Pain, Fifth Ed., American, Pain Society (2003); Evidence Based Report of the U.S. Agency for Healthcare Research and Quality (AHRQ) on the Management of Cancer Pain, Report No. 35, AHRQ Publication No. 02-E002, October 2001; Can et al. J Nat Cancer Inst Monograph 2004; 32:23-31; Agency for Health Care Policy and Research Clinical Practice Guidelines for Cancer Pain Management, Guideline No. 9, AHCPR Publication No. 94-0592, March 1994; Agency for Health Care Policy and Research Clinical Practice Guideline for Acute Pain Management, Guideline No. 1, AHCPR Publication No. 92-0032, February, 1992; Guideline for the Management of Cancer Pain in Adults, American Pain Society, 2005; Guideline for the Management of Pain in Osteoarthritis, Rheumatoid Arthritis, and Juvenile Chronic Arthritis, 2^{nd} Ed., American Pain Society, 2002), e.g., morphine, codeine, oxycodone, oxymorphone, hydromorphone, methadone, hydrocodone).

It is an object of certain preferred embodiments of the present invention to treat pain in patients who have a suboptimal efficacy or safety response with other orally approved extended release opioids (e.g., MS Contin®, Kadian®, Avinza®, Ultram® ER, Opana® ER, Palladone®, Jurnista®.

There is a need for oral formulations of buprenorphine that are therapeutically effective for the treatment of various medical conditions, including but not limited to pain, cough, dyspnea and opioid addiction disorders.

To the applicant's knowledge, there are no data demonstrating the efficacy of any oral pharmaceutical compositions of buprenorphine for the treatment of any medical disorder including cancer pain, neuropathic pain, cough, dyspnea, restless leg syndrome, acute herpes zoster, visceral pain and breakthrough pain.

To the applicant's knowledge, there are no data demonstrating the efficacy of any oral extended release pharmaceutical compositions of buprenorphine for the treatment of any pain state, opioid dependence, addiction disorders and other buprenorphine responsive medical conditions.

In some embodiments, the present invention is directed at oral pharmaceutical composition for the treatment of pain, opioid dependence, addiction disorders, cough, dyspnea, restless leg syndrome and other buprenorphine responsive medical conditions comprising a therapeutically effective amount of buprenorphine or a pharmaceutically acceptable salt of buprenorphine, or a mixture thereof.

The present invention relates to oral buprenorphine pharmaceutical compositions and methods for the treatment of pain, including acute pain, chronic pain, cancer pain, neuropathic pain, cough, dyspnea, acute herpes zoster, visceral pain and breakthrough pain.

The present invention relates to oral buprenorphine pharmaceutical compositions and methods for the treatment of acute and chronic cough, including iatrogenic cough, post-infectious cough, cough secondary to asthma, COPD, lung cancer, gastroesophageal reflux disease, respiratory bacterial and viral infections, and upper airway cough syndrome.

The present invention relates to oral buprenorphine pharmaceutical compositions and methods for the treatment of urinary incontinence.

The present invention relates to oral buprenorphine pharmaceutical compositions and methods for the treatment of restless leg syndrome.

The present invention also relates to oral buprenorphine pharmaceutical compositions and methods for the treatment of addiction disorders.

The present invention relates to oral buprenorphine pharmaceutical compositions and methods for the treatment of conditions other than pain and addiction disorders amenable to treatment with buprenorphine.

It is an object of certain preferred embodiments of the present invention to substantially improve the efficiency and quality of pain management in human patients experiencing pain which is unresponsive or suboptimally responsive to opioids classified as Schedule II (C-II) opioids under the United States Controlled Substance Act and regulations (as amended).

It is an object of some preferred embodiments to provide an oral pharmaceutical composition comprising a therapeutically effective amount of buprenorphine which produces less moderate to severe sedation or drowsiness than after an equal amount or dose of commercially available immediate release sublingual formulation of buprenorphine listed in FDA's Orange Book or an immediate release dosage form (e.g., solution, suspension, tablet or capsule) given orally.

It is an object of some preferred embodiments to provide an oral pharmaceutical composition comprising a therapeutically effective amount of buprenorphine which produces less moderate to severe nausea than after an equal amount or dose of commercially available immediate release sublingual formulation of buprenorphine listed in FDA's Orange Book or an immediate release dosage form (e.g., solution, suspension, tablet or capsule) given orally.

It is an object of some preferred embodiments to provide an oral pharmaceutical composition comprising a therapeutically effective amount of buprenorphine which produces less dizziness than after an equal amount or dose of commercially available immediate release sublingual formulation of buprenorphine listed in FDA's Orange Book or an immediate release dosage form (e.g., solution, suspension, tablet or capsule) given orally.

It is an object of some preferred embodiments to provide an oral pharmaceutical composition comprising a therapeutically effective amount of buprenorphine which produces less dry mouth than after an equal amount or dose of commercially available immediate release sublingual formulation of buprenorphine listed in FDA's Orange Book or an immediate release dosage form (e.g., solution, suspension, tablet or capsule) given orally.

It is an object of some preferred embodiments to provide an oral pharmaceutical composition comprising a therapeutically effective amount of buprenorphine which has less abuse potential than an equal amount or dose of commercially available immediate release sublingual formulation of buprenorphine listed in FDA's Orange Book or an immediate release dosage form (e.g., solution, suspension, tablet or capsule) given orally (e.g., produces lower abuse scores for “drug effects”, “drug liking”, “coasting”, “take again”, as defined herein).

It is an object of some preferred embodiments to provide an oral pharmaceutical composition comprising a therapeutically effective amount of buprenorphine which produces less neurologic, cognitive, motor and psychomotor impairment than an equal amount or dose of commercially available immediate release sublingual formulation of buprenorphine listed in FDA's Orange Book or an immediate release dosage form (e.g., solution, suspension, tablet or capsule) given orally (e.g., produces lower impairment scores for “critical tracking task”, “stop signal task” and “Tower of London” (TOL), as defined herein).

It is an object of certain preferred embodiments of the present invention to provide bioavailable oral buprenorphine formulations which provide a substantially increased duration of effect as compared to immediate release buprenorphine formulations.

It is an object of certain preferred embodiments of the present invention to provide bioavailable formulations for oral administration suitable for up to every 1, 2, 4, 6, 8, 12, 24, 36, 48 hour and 72 hour administration.

It is an object of certain preferred embodiments of the present invention to provide bioavailable formulations of buprenorphine for oral administration suitable for up to every 1, 2, 4, 6, 8, 12, 24, 36, 48 hour and 72 hour administration which provide an early onset and sustained duration of therapeutic effect.

It is an object of certain preferred embodiments of the present invention to provide bioavailable formulations of buprenorphine for oral administration suitable for up to every 1, 2, 4, 6, 8, 12, 24, 36, 48 hour and 72 hour administration which provide an early onset and sustained duration of therapeutic effect, without the need for a loading dose.

It is an object of certain preferred embodiments of the present invention to provide oral buprenorphine formulations.

It is an object of certain preferred embodiments of the present invention to provide oral buprenorphine formulations which provide a therapeutic effect for up to about 0.5, 1, 2, 4, 6, 8, 12, 24, 36, 48 hour and 72 hours.

It is an object of certain preferred embodiments of the invention to provide an oral buprenorphine formulation which provides a sustained duration of therapeutic effect.

It is an object of certain preferred embodiments of the invention to provide an oral buprenorphine formulation which provides an early onset and sustained duration of therapeutic effect.

It is an object of certain preferred embodiments of the invention to provide a method and formulations of oral buprenorphine for the treatment of pain.

It is an object of certain preferred embodiments of the invention to provide a method and formulations of oral buprenorphine for the treatment of opioid addiction disorders.

It is an object of certain preferred embodiments of the invention to provide a method and formulations of oral buprenorphine for the treatment of opioid dependence.

It is an object of certain preferred embodiments of the invention to provide a method and formulations of oral buprenorphine intended as therapeutic substitutes for unprescribed, illicit, or medically unsanctioned pharmaceutical grade or street grade opioids.

It is an object of certain preferred embodiments of the invention to provide a method and formulations of oral buprenorphine intended as part of an opioid substitution or opioid maintenance therapy in patients with an opioid addiction disorder or a polysubstance abuse disorder.

It is an object of certain preferred embodiments of the invention to provide a method and formulations of oral buprenorphine for the treatment of conditions other than pain and addiction disorders.

It is an object of certain preferred embodiments of the invention to provide a method and formulations of oral buprenorphine, said formulations and methods not having a propensity of substantial drug accumulation.

It is an object of certain preferred embodiments of the invention to provide a method and formulations of oral buprenorphine, said formulations having a reduced potential for drug abuse and drug diversion.

It is an object of certain preferred embodiments of the invention to provide a method and formulations of oral buprenorphine for the treatment of cough, dyspnea, restless leg syndrome, acute herpes zoster, visceral pain, breakthrough pain, opioid dependence and urinary incontinence.

In some preferred embodiments one or more or all of the specifications, embodiments and claims of the invention applicable to a human subject are also applicable to other mammals.

It is an object of certain preferred embodiments of the invention to provide a method and formulations of oral buprenorphine, said formulations having a reduced intrasubject pharmacokinetic variability.

It is an object of certain preferred embodiments of the invention to provide a method and formulations of oral buprenorphine, said formulations having a reduced intersubject pharmacokinetic variability.

It is an object of certain preferred embodiments of the invention to provide a method and formulations of oral buprenorphine, said formulations having a reduced peak to trough fluctuation.

It is an object of certain preferred embodiments of the invention to provide a method and formulations of oral buprenorphine, said formulations having a shorter time to therapeutic concentrations or having a shorter time to steady-state.

It is an object of certain preferred embodiments of the invention to provide a method and formulations of oral buprenorphine for the treatment of pain, said formulations suitable for use in acute pain, including acute postsurgical pain.

It is an object of certain preferred embodiments of the invention to provide a method and formulations of oral buprenorphine for the treatment of pain, said formulations suitable for use in chronic pain.

It is an object of certain preferred embodiments of the invention to provide a method and formulations of oral buprenorphine for the treatment of pain, said formulations suitable for use in cancer pain.

It is an object of certain preferred embodiments of the invention to provide a method and formulations of oral buprenorphine for the treatment of pain, said formulations suitable for use in neuropathic pain.

It is an object of certain preferred embodiments of the invention to provide a method and formulations of oral buprenorphine for the treatment of pain, said formulations suitable for use in somatic, visceral and idiopathic pain.

It is an object of certain preferred embodiments of the invention to provide a method and formulations of oral buprenorphine for the treatment of pain, said formulations suitable for use in breakthrough pain or various etiologies, including cancer, chronic pain and neuropathic pain.

In some preferred embodiments, the invention comprises pharmaceutical composition comprising a therapeutically effective amount of buprenorphine or a pharmaceutically acceptable salt of buprenorphine or a mixture thereof wherein all the specifications of the invention applicable to the treatment of pain and addiction disorders are also applicable to the treatment of medical conditions other than pain and addiction disorders.

In some preferred embodiments, the invention provides oral buprenorphine formulations which when evaluated versus other buprenorphine dosage forms in fasted healthy subjects provide a relative mean Cₘₐₓ whose 90% confidence interval is outside the 80.00% to 125.00, under single-dose fasted test conditions in healthy subjects.

In some preferred embodiments, the invention provides oral buprenorphine formulations which when evaluated versus other buprenorphine dosage forms in fasted healthy subjects provide a relative mean AUC_{0-τ} whose 90% confidence interval is outside the 80.00% to 125.00, under single-dose fasted test conditions in healthy subjects.

In some preferred embodiments, the invention provides oral buprenorphine formulations which when evaluated versus other buprenorphine dosage forms in fasted healthy subjects provide a relative mean AUC_{0-inf} whose 90% confidence interval is outside the 80.00% to 125.00, under single-dose fasted test conditions in healthy subjects.

Specifically excluded from this invention are buprenorphine dosage forms which are administered by the lingual, sublingual, oro-mucosal, transmucosal and buccal routes. Lingual, sublingual, oro-mucosal, transmucosal and buccal routes are intended to provide absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach). Such formulations and their method of administration are well known in the art and include lozenges, transmucosal films, buccal products, mucoretentive products, orally disintegrating tablets, fast dissolving tablets, fast dispersing tablets, fast disintegrating dosage forms, provided they are administered for absorption or substantial absorption of the drug in the oral cavity (i.e., the mouth) through rapid or slow dissolution in the oral cavity and/or through longer residence in the oral cavity (i.e., oral cavity residence beyond the usual time associated with oral ingestion of drug intended to be deposited into the stomach).

Some or all of the above objects and others are achieved by embodiments of the present invention, which is directed in part to a dosage form of oral buprenorphine.

In some preferred embodiments, the dosage form of oral buprenorphine releases substantially more buprenorphine into systemic circulation during the first half of the intended dosing frequency than during the second half of the intended dosing frequency.

In some preferred embodiments, the dosage form of oral buprenorphine releases at least as much buprenorphine into systemic circulation during the first one-third of the intended dosing frequency as during the remainder of the intended dosing frequency.

In some preferred embodiments, the dosage form of oral buprenorphine releases substantially more buprenorphine into systemic circulation during the first one-third of the intended dosing frequency than during the remainder of the intended dosing frequency.

In some preferred embodiments, the invention comprises an oral pharmaceutical composition for the prevention or treatment of pain comprising a therapeutically effective amount of buprenorphine or a pharmaceutically acceptable salt of buprenorphine or a mixture thereof.

It is an object of certain preferred embodiments of the invention to provide a method and formulations of oral buprenorphine, said formulations not having a propensity for toxicity.

It is an object of certain embodiments of the present invention to provide oral buprenorphine formulations in immediate release form.

It is an object of certain embodiments of the present invention to provide oral buprenorphine formulations in controlled release form.

It is an object of certain embodiments of the present invention to provide oral buprenorphine formulations with both immediate release and controlled release forms.

It is an object of certain embodiments of the present invention to provide oral buprenorphine formulations in pulsatile release form.

It is an object of certain embodiments of the present invention to provide oral buprenorphine wherein the buprenorphine is dispersed within a matrix.

In certain preferred embodiments the oral dosage form of the present invention comprises a matrix which includes a sustained release material and buprenorphine or a pharmaceutically acceptable salt thereof. In certain preferred embodiments, the matrix is compressed into a tablet and may be optionally overcoated with a coating that in addition to the sustained release material of the matrix may control the release of the buprenorphine or pharmaceutically acceptable salt thereof from the formulation, such that blood levels of active ingredient are maintained within the therapeutic range over an extended period of time. In certain alternate embodiments, the matrix is encapsulated.

In certain preferred embodiments, the sustained release oral dosage form of the present invention comprises a plurality of pharmaceutically acceptable sustained release matrices comprising buprenorphine, the dosage form maintaining the blood plasma levels of buprenorphine within the therapeutic range over an extended period of time when administered to patients.

In some preferred embodiments, the dosage form of the invention comprises oral buprenorphine formulated to release the buprenorphine from the dosage form or to initiate the release of the buprenorphine from the dosage form after a certain specific amount of time post-oral ingestion, or at an approximately specific anatomic location in the gastrointestinal tract, or when the dosage form is in contact with specific gastrointestinal conditions (e.g., pH range, osmolarity, electrolyte content, food content, pressure, time since first ingestion, osmotic pressure in the dosage form, osmotic pressure in the gastrointestinal tract, hydration, etc), said dosage form suitable for providing an orally effective therapeutic for a short, intermediate or extended duration of effect, said dosage form providing a rapid or delayed onset of clinical effect.

In certain preferred embodiments the sustained release oral dosage form of the present invention is an osmotic dosage form which comprises a single layer or bilayer core comprising buprenorphine; an expandable polymer; a semipermeable membrane surrounding the core; and a passageway disposed in the semipermeable membrane for sustained release of the buprenorphine or pharmaceutically acceptable salt thereof, such that blood levels of active ingredient are maintained within the therapeutic range over an extended period of time when administered to patients.

Other oral osmotic delivery systems may be used for the oral administration of buprenorphine.

In some preferred embodiments of the invention, the oral buprenorphine is interdispersed and are not isolated from each other in two distinct layers.

In some preferred embodiments of the invention, the oral buprenorphine is in the form of multiparticulates.

In some preferred embodiments of the invention, the oral buprenorphine is dispersed in a matrix

In some preferred embodiments of the invention, the oral buprenorphine is in the form of multiparticulates can be dispersed in a matrix or contained in a capsule.

In some preferred embodiments of the invention, the oral buprenorphine is in the form of multiparticulates can be dispersed in a matrix and compressed into a tablet.

In some preferred embodiments of the invention, the oral buprenorphine is in a matrix that is in the form of pellets.

In some preferred embodiments of the invention, the oral buprenorphine is in coated beads.

In some preferred embodiments, the dosage form of the invention comprises a compressed tablet, compressed capsule or uncompressed capsule. In other embodiments, the dosage form comprises a liquid fill capsule.

In some preferred embodiments, the oral dosage in immediate or sustained release form provides therapeutic effects that persist despite the low or undectable buprenorphine concentrations.

In some preferred embodiments, the dosage form of the invention comprises an oral formulation (e.g., tablet or capsule) which is coated to prevent substantial direct contact of buprenorphine with oral cavity (e.g. tongue, oral mucosa), oropharyngeal mucosal surface, esophagus or stomach.

In some preferred embodiments, the dosage form of the invention comprises an oral formulation which is coated with a film or polymer. In some preferred embodiments, the dosage form of the invention comprises buprenorphine in an enteric coating.

In some preferred embodiments, the dosage form provides an oral pharmaceutical composition comprising a therapeutically effective amount of buprenorphine; said therapeutically effective amount in a reservoir comprising: (i) buprenorphine or a pharmaceutically acceptable salt of buprenorphine, or a mixture thereof; (ii) a membrane layer, said membrane being substantially permeable to buprenorphine; wherein the dosage form substantially releases the buprenorphine from the dosage form to render said dosage form suitable for extended release to a human patient.

In some preferred embodiments, the dosage form provides an oral pharmaceutical composition comprising a plurality of pharmaceutically acceptable beads coated with a therapeutically effective amount of buprenorphine or a pharmaceutically acceptable salt of buprenorphine, or a mixture thereof; and overcoated with controlled release material to render said dosage form suitable for extended release oral administration to a human patient.

In some preferred embodiments, the dosage form provides an oral pharmaceutical composition comprising (i) a drug layer comprising a therapeutically effective amount of buprenorphine; and (ii) a displacement layer comprising an osmopolymer; and (b) a semipermeable wall surrounding the bilayer core having a passageway disposed therein for the release of said buprenorphine or a pharmaceutically acceptable salt thereof; said dosage form suitable for extended release oral administration to a human patient.

In some preferred embodiments, the oral dosage form is a controlled release material suitable for extended release in a human patient of the dosage form comprises a matrix. In some preferred embodiments, the said matrix is a plurality of multiparticulate matrices. In some preferred embodiments, the multiparticulates are compressed into a tablet. In some preferred embodiments, the multiparticulates are disposed in a pharmaceutically acceptable capsule.

In some preferred embodiments, the controlled release material of the oral dosage form of the invention is selected from the group consisting of hydrophobic polymers, hydrophilic polymers, gums, protein derived materials, waxes, shellac, oils, fats and mixtures thereof.

In some preferred embodiments, the controlled release material of the oral dosage form of the invention is selected from the group consisting of polyethylene oxide, polyvinyl alcohol, hydroxypropyl methyl cellulose, a carbomer and mixtures thereof.

In some preferred embodiments, the controlled release material of the oral dosage form of the invention is selected from the group consisting of hydrogenated Type I or Type II vegetable oils, polyoxyethylene stearates and distearates, glycerol monostearate, and non-polymeric, non-water soluble liquids carbohydrate-based substances or poorly water soluble, high melting point (mp=40 to 100° C.) waxes and mixtures thereof.

In some preferred embodiments, the oral dosage form comprises a plurality of pharmaceutically acceptable beads coated with drug and overcoated with controlled release material.

In some preferred embodiments, the oral dosage form comprises (i) a drug layer; and (ii) a displacement layer comprising an osmopolymer; and (b) a semipermeable wall surrounding the bilayer core having a passageway disposed therein for the release of said drug.

In some preferred embodiments, the oral dosage form comprises a compressed tablet, compressed capsule or uncompressed capsule. In some preferred embodiments, the oral dosage form comprises a liquid fill capsule.

In some preferred embodiments, the in vivo pharmacokinetic parameters of the specifications, embodiments and claims are derived or determined under fed conditions. In other preferred embodiments, the in vivo pharmacokinetic parameters are derived or determined under fasted conditions.

In some preferred embodiments, the present invention excludes immediate release dosage forms.

In some preferred embodiments, the present invention excludes dosage forms devoid or material to render the dosage form as modified release, delayed release, extended release or controlled release.

In some embodiments of the invention, the oral dosage forms of buprenorphine exclude pharmaceutical compositions buprenorphine which when coated or encapsulated with any excipient where the coated or encapsulated particle is less than about 130 μm, or 200 μm, or 250 μm, or 400 μm, or 1000 μm, or 2000 μm, or 3000 μm, or 3500 μm. In some embodiments of the invention, the oral dosage forms of buprenorphine exclude oral pharmaceutical compositions of buprenorphine which contain polyvinyl alcohol, or polyvinylpyrrolidone, or block copolymers of propylene oxide or ethylene oxide, or polyethylene glycol or tetrafunctional block copolymers derived from sequential addition of propylene oxide and ethylene oxide to ethylenediamine. In some embodiments of the invention, the oral dosage forms of buprenorphine exclude oral pharmaceutical compositions of buprenorphine where the buprenorphine is encapsulated by polyvinyl alcohol, or polyvinylpyrrolidone, or block copolymers of propylene oxide or ethylene oxide, or polyethylene glycol or tetrafunctional block copolymers derived from sequential addition of propylene oxide and ethylene oxide to ethylenediamine. In some embodiments of the invention, the oral dosage forms of buprenorphine exclude oral pharmaceutical compositions of buprenorphine where the buprenorphine is encapsulated by polyvinyl alcohol, or polyvinylpyrrolidone, or block copolymers of propylene oxide or ethylene oxide, or polyethylene glycol or tetrafunctional block copolymers derived from sequential addition of propylene oxide and ethylene oxide to ethylenediamine, said encapsulated particles having a particle of less than about 3000 μm, or 3500 μm. In some embodiments of the invention, the oral dosage forms of buprenorphine exclude pharmaceutical compositions buprenorphine which are encapsulated with polyethylene glycol. In some embodiments of the invention, the oral dosage forms of buprenorphine exclude pharmaceutical compositions buprenorphine which are encapsulated with polyethylene glycol, where the buprenorphine content of the composition is between about 2 parts in 1000 to about 4 parts in 100, or between about 1.5 parts in 1000 to about 4.5 parts or 5 parts in 100.

In some embodiments of the invention, the oral dosage form of the invention exclude oral immediate release forms of buprenorphine. In some embodiments of the invention, the oral dosage form of the invention excludes oral immediate release dosage forms of buprenorphine which have been modified to enhance the solubility or bioavailability of the buprenorphine in the upper GI tract (e.g., by use of certain excipients or by physical manipulation of the buprenorphine or granulation), using methods well know in the art, including complexation (e.g., with cyclodextrins), or particle size reduction (e.g., micronization), or lipid suspensions, solutions, emulsions, microemulsions, or mixed micelles, or self-emulsifying drug delivery systems (SEDDS), or self-microemulsifying drug delivery systems (SMEDDS), or thixotropic vehicles, or surfactants, or solid dispersions, or liposomes, or co-solvents, or solvation.

In some embodiments of the invention, the oral dosage form of the invention excludes oral dosage forms of buprenorphine which have been modified to enhance the bioavailability of the buprenorphine in the upper GI tract.

In some embodiments of the invention, the oral dosage forms of buprenorphine are limited to controlled release buprenorphine. In some embodiments of the invention, the oral dosage forms of buprenorphine are limited to controlled release buprenorphine which are matrix or monolithic matrix formulations. In some embodiments of the invention, the oral dosage forms of buprenorphine exclude multiparticulate matrix controlled release buprenorphine formulations.

In some embodiments of the invention, the oral dosage forms of buprenorphine excludes controlled release buprenorphine formulations where the buprenorphine is in controlled release multiparticulates or controlled release microparticulates, where the buprenorphine is not entirely coated with polymer. In some embodiments of the invention, the oral dosage forms of buprenorphine excludes controlled release buprenorphine formulations where the controlled release buprenorphine multiparticulates or microparticulates are less than about 130 μm, or 140 μm, or 150 μm, or 160 μm, or 180 μm, or 200 μm, or 250 μm, or 300 μm, or 350 μm, or 400 μm, or 450 μm, or 500 μm, or 550 μm, or 600 μm, or 650 μm.

In some embodiments of the invention, the oral dosage forms of buprenorphine are limited to controlled release buprenorphine which provide first release or first therapeutically beneficial release of buprenorphine not until about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion. In some embodiments of the invention, the oral dosage forms of buprenorphine are limited to controlled release buprenorphine which are overcoated with a pH sensitive material which dissolve at a pH <4, or pH <4.5, or pH, <5, or pH <5.5, or pH<6, or pH <6.5, or pH <6.8, or pH <7, or pH <7.2.

In some embodiments of the invention, the oral dosage form of the invention excludes oral controlled release dosage forms of buprenorphine which have been modified to enhance the solubility or bioavailability of the buprenorphine in the upper GI tract through complexation (e.g., with cyclodextrins), or particle size reduction (e.g., micronization), or lipid suspensions, solutions, emulsions, microemulsions, mixed micelles, or self-emulsifying drug delivery systems (SEDDS), or self-microemulsifying drug delivery systems (SMEDDS), or thixotropic vehicles, or surfactants, or solid dispersions, or liposomes, or co-solvents, or solvation. In some embodiments of the invention, the oral dosage forms of buprenorphine are limited to modified release buprenorphine which provides an onset of therapeutic effect not before about 1, 1, 5, 2, 2.5 3, 3.5, 4, 4.5, 5, 5.5 or 6 hours following first ingestion.

In some embodiments of the invention, the oral dosage forms of buprenorphine are limited to modified release buprenorphine which provides quantifiable or therapeutic or substantial plasma concentrations not before about 1, 1, 5, 2, 2.5 3, 3.5, 4, 4.5, 5, 5.5 or 6 hours following first ingestion. In some embodiments of the invention, the oral dosage forms of buprenorphine are limited to modified release buprenorphine which provide quantifiable, or substantial plasma concentrations or therapeutic plasma concentrations not before about 1, 1, 5, 2, 2.5 3, 3.5, 4, 4.5, 5, 5.5 or 6 hours following first ingestion. In some embodiments of the invention, the oral dosage forms of buprenorphine are limited to modified release buprenorphine which are osmotic delivery dosage forms (e.g., push pull osmotic pumps, monolithic osmotic delivery systems and controlled porosity osmotic pumps).

In some embodiments of the invention, the oral dosage forms of buprenorphine are limited to modified release buprenorphine which are osmotic delivery dosage forms, overcoated with a pH sensitive material which dissolve at a pH <4, or pH <4.5, or pH, <5, or pH <5.5, or pH <6, or pH <6.5, or pH <6.8, or pH <7, or pH <7.2. In some embodiments of the invention, the oral dosage forms of buprenorphine are limited to modified release buprenorphine which provide release of all, substantially all, or most of the buprenorphine only at pH>4, or pH>4.5, or pH, >5, or pH>5.5, or pH>6, or pH>6.5, or pH>7, or pH >7.5, or pH>7.8 upon dissolution using the USP Paddle Method for 2 hours at 37° C. at 100 rpm in 900 mL distilled water adjusted for pH.

In some embodiments of the invention, the oral dosage forms of buprenorphine are limited to modified release buprenorphine which provide release of all, substantially all, or most of the buprenorphine distal to the stomach, distal to the duodenum, distal to the jejunum, distal to the ileum, or distal to the ileo-cecal junction. In some embodiments of the invention, the oral dosage forms of buprenorphine are limited to modified release buprenorphine which provide release of all, substantially all, or most of the buprenorphine at least about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion. In some embodiments of the invention, the oral dosage forms of buprenorphine are limited to modified release buprenorphine which begin the release of all, substantially all, most, or some of the buprenorphine at least about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion.

In some embodiments of the invention, the oral dosage forms of buprenorphine are limited to modified release buprenorphine which provide the intended therapeutic effect not until about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion. In some embodiments of the invention, the oral dosage forms of buprenorphine are limited to modified release buprenorphine which provide all, substantially all, most, or some of the intended therapeutic effect not until about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion. In some embodiments of the invention, the oral dosage forms of buprenorphine are limited to modified release buprenorphine which begin to provide all, substantially all, most, or some of the intended therapeutic effect not until about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion.

In some embodiments of the invention, the oral dosage forms of buprenorphine are limited to modified release buprenorphine which provide a psychic effect in recreational drug or opioid users and drug or opioid abusers not until about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion. In some embodiments of the invention, the oral dosage forms of buprenorphine are limited to modified release buprenorphine provide a psychic effect in recreational drug or opioid users and drug or opioid abusers not until about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion. In some embodiments of the invention, the oral dosage forms of buprenorphine are limited to modified release buprenorphine which begin to provide a psychic effect in recreational drug or opioid users and drug or opioid abusers not until about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion.

In some embodiments of the invention, the oral dosage forms of buprenorphine are limited to modified release buprenorphine which result in little or no nausea, or little or no sedation until about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion. In some embodiments of the invention, the oral dosage forms of buprenorphine are limited to modified release buprenorphine which result in little or no nausea, or little or no sedation until about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion. In some embodiments of the invention, the oral dosage forms of buprenorphine are limited to modified release buprenorphine which result in little or no nausea, or little or no sedation until about 1, 1.5, 2, 2.5, 3, 3.5, 4, 4.5, 5, 5.5, 6, 6.5, 7, 7.5, or 8 hours after first oral ingestion, preferably at least about 2, 2.5, 3, 3.5, or 4 hours after first oral ingestion.

In some embodiments of the invention, the oral dosage forms of buprenorphine are limited to modified release buprenorphine which provide all, substantially all, most, or some of the release of buprenorphine from the dosage form distal to the stomach, or distal to the duodenum, or distal to the jejunum, or distal to the ileum, or distal to the ileo-cecal junction, or in the colon. In some embodiments of the invention, the oral dosage forms of buprenorphine are limited to modified release buprenorphine which begins to provide all, substantially all, most, or some of the release of buprenorphine from the dosage form distal to the stomach, or distal to the duodenum, or distal to the jejunum, or distal to the ileum, or distal to the ileo-cecal junction, or in the colon. In some embodiments of the invention, the oral dosage forms of buprenorphine are limited to modified release buprenorphine which provide release of buprenorphine from the dosage form in the duodenum and jejunum, or in the duodenum, jejunum and ileum, or in duodenum, jejunum, ileum and colon. In some embodiments of the invention, the oral dosage forms of buprenorphine are limited to modified release buprenorphine which provide release of buprenorphine from the dosage form in the ileum and colon. In some embodiments of the invention, the oral dosage forms of buprenorphine are limited to modified release buprenorphine which provide release of buprenorphine from the dosage form in the ileo-colonic region. In some embodiments of the invention, the oral dosage forms of buprenorphine are limited to modified release buprenorphine which provide release of buprenorphine from the dosage form in the colon.

In some embodiments of the invention, the oral dosage forms of buprenorphine are limited to buprenorphine without an additional therapeutic agent (e.g., acetaminophen, COX-2 inhibitor, NSAID, methadone) incorporated in the dosage form or given concurrently with the dosage form of the invention. In some embodiments of the invention, the oral dosage forms of buprenorphine are limited to buprenorphine without an additional therapeutic agent to treat the same medical condition or to enhance the effect of the buprenorphine which is incorporated in the dosage form or given concurrently with the dosage form of the invention.

In some embodiments, the invention excludes oral buprenorphine pharmaceutical compositions which provide a more rapid onset of action of buprenorphine compared with sublingual buprenorphine, or orally ingested buprenorphine active pharmaceutical ingredient (API), or oral buprenorphine made using conventional excipients. In some embodiments, the invention excludes oral controlled-release formulations buprenorphine pharmaceutical compositions which a more rapid onset of action compared with sublingual buprenorphine, buccal buprenorphine, or oral buprenorphine API or oral immediate release buprenorphine.

In some embodiments, the invention excludes oral buprenorphine pharmaceutical compositions for the treatment of acute pain. In some embodiments, the invention excludes oral buprenorphine pharmaceutical compositions for the treatment of addiction disorders.

In some embodiments of the invention, the invention excludes oral buprenorphine pharmaceutical compositions which provide more rapid (or “improved”) dissolution of the buprenorphine when compared with sublingual buprenorphine, or orally ingested buprenorphine active pharmaceutical ingredient (e.g., uncompressed powder in a capsule), or oral immediate release buprenorphine made using conventional excipients. In some embodiments, the invention excludes oral buprenorphine pharmaceutical compositions which provide greater than 65%, or greater than 75%, or greater than 85% release of buprenorphine form the dosage form when measured by USP Paddle Method at 50 or 100 rpm in 900 mL of 0.1 N HCl buffer (at any pH between 1.6 and 3) at 37° C. after 45 minutes. In some embodiments, the invention excludes oral buprenorphine pharmaceutical compositions which provide greater than about 50%, or about 60% or about 70%, or about 80% release of buprenorphine form the dosage form when measured by USP Paddle Method at 50 or 100 rpm in 900 mL of pH 6.8 buffer at 37° C. after 60 minutes.

In some embodiments of the invention, the oral dosage form of buprenorphine contains an antioxidant in a molar ratio between antioxidant and buprenorphine greater than 3:1. In some embodiments of the invention, the oral dosage forms of buprenorphine exclude oral pharmaceutical compositions buprenorphine which contain an antioxidant in any amount. In some embodiments of the invention, the oral dosage form contains an antioxidant which is sequestered or substantially sequestered in the push layer of the dosage form. In some embodiments of the invention, the oral dosage form contains no antioxidant in the buprenorphine containing pull layer or reservoir but does contain an antioxidant in the push layer of the dosage form. In some embodiments of the invention, the oral dosage form contains an antioxidant which is not commingled or interspersed with the buprenorphine. In some embodiments of the invention, the oral dosage form contains an antioxidant which is not in contact or substantial contact with the buprenorphine. In some embodiments of the invention, the oral dosage form contains an antioxidant which does not provide anti-oxidant properties to the buprenorphine or protect the buprenorphine from oxidation by virtue of being in a separate compartment (e.g., push layer of an osmotic delivery system), said anti-oxidant protecting other excipients in the separate compartment (e.g., push layer) from oxidation.

In some embodiments of the invention, the oral dosage forms of buprenorphine is devoid of antioxidants selected from the group comprising ascorbic acid, or vitamin E, or tocopherol, or sodium metabisulfite, or butylated hydroxyanisole, or butylated hydroxytoluene, or alpha-lipoic acid, and mixtures thereof. In some embodiments of the invention, the oral dosage forms of buprenorphine is devoid of antioxidants selected from the group comprising ascorbic acid, or ascorbyl palmitate, or butylated hydroxyanisole, or butylated hydroxytoluene, or hypophosphorous acid, or monothioglycerol, or potassium metabisulfite, or propyl gallate, or sodium bisulfate, or sodium formaldehyde sulfoxylate, or sodium metabisulfite, or sodium sulfite, or sodium thiosulfate, or sulfur dioxide, or tocopherol, or tocopherols excipient and mixtures thereof. In some embodiments of the invention, the oral dosage forms of buprenorphine are devoid of antioxidants. In some embodiments of the invention, the oral dosage forms of buprenorphine do not contain antioxidants in a molar ratio between antioxidant and buprenorphine from 1:1 to 3:1 or in a molar ratio of antioxidant and buprenorphine ranging which is less than 1:1. In some embodiments, the foregoing limitation in amount or any use of antioxidant refers to ascorbic acid, or vitamin E, or tocopherol, or sodium metabisulfite, or butylated hydroxyanisole, or butylated hydroxytoluene, or alpha-lipoic acid, and mixtures thereof.

In some embodiments of the invention, the oral dosage forms of buprenorphine include magnesium.

In some embodiments of the invention, the oral dosage forms of buprenorphine exclude chelating agents. In some embodiments, the foregoing limitation in any use of chelating agent refers to is limited, said chelating agent is edetic acid, or malic acid, and mixtures thereof.

Some or all of the objects and others are achieved by embodiments of the present invention, which is directed in part to a dosage form of oral buprenorphine.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of chronic pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of neuropathic pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of cancer pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain, excluding acute pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain, excluding acute postsurgical pain.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain which is unresponsive to other oral formulations of opioid analgesics, particularly full or pure mu opioid agonists.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain which is unresponsive to other oral formulations of pure or full mu-opioid receptor agonists.

In some preferred embodiments, the dosage form provides an oral pharmaceutical composition comprising a therapeutically effective amount of buprenorphine or a pharmaceutically acceptable salt of buprenorphine or a mixture thereof; said dosage form intended for the treatment of pain which is unresponsive to sublingual buprenorphine.

In some preferred embodiments, the dosage form provides an oral pharmaceutical composition comprising a therapeutically effective amount of buprenorphine or a pharmaceutically acceptable salt of buprenorphine or a mixture thereof; said dosage form intended for the treatment of pain which is unresponsive to transdermal buprenorphine.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of pain in patients with an addiction disorder or at significant risk of an addiction disorder.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of addiction disorders.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of opioid addiction disorders.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment of addiction disorders unresponsive to methadone.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for treatment unresponsive to sublingual, lingual, or buccal buprenorphine.

In some preferred embodiments, the dosage form provides an oral extended release pharmaceutical composition comprising a therapeutically effective amount of buprenorphine or a pharmaceutically acceptable salt of buprenorphine or a mixture thereof; said dosage form intended solely for treatment unresponsive to oral immediate release buprenorphine.

In some preferred embodiments, the dosage form provides an oral pharmaceutical composition comprising a therapeutically effective amount of buprenorphine or a pharmaceutically acceptable salt of buprenorphine or a mixture thereof; said dosage form intended solely for the treatment unresponsive to buprenorphine dosage forms intended to be significantly absorbed through the oral cavity oral mucosa (e.g., lozenges, orally disintegrating tablets, fast dissolving tablets, effervescent tablets, buccal dosage forms, sublingual dosage forms, lingual dosage forms or muco-retentive dosage forms),

The invention is also directed to kits of the dosage forms, including kits for titration disclosed herein.

In another aspect, the invention relates to a method for prevention or treatment of pain, cough, dyspnea, opioid addiction disorders, restless leg syndrome, acute herpes zoster, visceral pain, breakthrough pain, opioid dependence and urinary incontinence, comprising oral administration of a dosage form containing buprenorphine or a pharmaceutically acceptable salt of buprenorphine or a mixture thereof.

In another aspect, the invention relates to a method for prevention or treatment of buprenorphine responsive or opioid responsive medical conditions, comprising oral administration of a dosage form containing buprenorphine or a pharmaceutically acceptable salt of buprenorphine or a mixture thereof.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said dosing regimen associated with reduced side effects, improved tolerability, improved efficiency of therapeutic response, reduced breakthrough symptoms (e.g., breakthrough pain) and reduced treatment discontinuation due to side effects.

In some preferred embodiments, the dosage form provides an oral pharmaceutical composition comprising a therapeutically effective amount of buprenorphine; optionally, an oral controlled release material to render said dosage form suitable for extended release, or delayed onset, extended release or delayed onset, rapid release or delayed onset, pulsatile release human patient; said dosage form administered at a prespecified dosing regimen; said dosing regimen associated with reduced side effects, improved tolerability, improved efficiency of therapeutic response, reduced breakthrough symptoms (e.g., breakthrough pain) and reduced treatment discontinuation due to side effects.

In some preferred embodiments, the dosage form provides an oral pharmaceutical composition comprising a therapeutically effective amount of buprenorphine; said dosage form suitable for oral immediate release in a human patient; said dosage form administered at a prespecified dosing regimen; said dosing regimen associated with reduced side effects, improved tolerability, improved efficiency of therapeutic response, reduced breakthrough symptoms (e.g., breakthrough pain) and reduced treatment discontinuation due to side effects.

In some preferred embodiments, the dosage form provides an oral pharmaceutical composition comprising a therapeutically effective amount of buprenorphine; optionally; said dosage form intended to treat pediatric patients; said dosage form administered at a prespecified dosing regimen; said dosing regimen as provided herein, except that the dose or total daily dose (as applicable) is multiplied by the ratio obtained from the child's weight in kilograms divided by 70 kilograms.

As used herein, “plasma T_{lag}” refers to a time period from first administration (or first dosing) of buprenorphine to the occurrence of first of two consecutive plasma buprenorphine concentrations of not less than 50 pg/mL per mg of administered buprenorphine base, provided that the second consecutive buprenorphine plasma concentration is obtained not more than 10 minutes and not less than 30 minutes after the first plasma buprenorphine concentration.

In some embodiments, the plasma T_{lag} of the oral buprenorphine dosage form is more than about 0.25, 0.5, 0.75, 1, 1.25, 1.5, 1.75, 2, 2.25, 2.5, 2.75, 3, 3.5, 4, 4.5, 5, 5.5, 6, 6.5, 7, 7.5, 8, 8.5, 9, 9.5, 10, 10.5, 11, 11.5 or 12 hours.

In some embodiments, where the oral dosage form of the invention provides duodenal delivery, jejunal delivery, ileal delivery, ileo-colonic delivery, or colonic delivery, the plasma T_{lag} of the oral buprenorphine dosage form is more than about 1.5, 2, 2.25, 2.5, 2.75, 3, 3.5, 4, 4.5, 5, 5.5, 6, 6.5, 7, 7.5, 8, 8.5, 9, 9.5, 10, 10.5, 11, 11.5 or 12 hours, more preferably, more than about 2, 2.25, 2.5, 2.75, 3, 3.5, 4, 4.5, 5, 5.5, or 6 hours.

In some embodiments, where the oral dosage form of the invention incorporates controlled release material, the plasma T_{lag} of the oral buprenorphine dosage form is more than about 1.5, 2, 2.25, 2.5, 2.75, 3, 3.5, 4, 4.5, 5, 5.5, 6, 6.5, 7, 7.5, 8, 8.5, 9, 9.5, 10, 10.5, 11, 11.5 or 12 hours, more preferably, more than about 2, 2.25, 2.5, 2.75, 3, 3.5, 4, 4.5, 5, 5.5, or 6 hours.

As used herein, “T_{lag(x)}” refers to the time from the start of dissolution testing to the first attainment of an in-vitro release of about 5% by weight of the active drug from the dosage form rate when measured by the USP Basket or Paddle Method at 100 rpm in 900 mL of dissolution media at 37° C., where “₍ₓ₎” is the pH of the dissolution media.

In some embodiments, the T_{lag(5.0)}, T_{lag(5.5)}, T_{lag(6.8)} T_{lag(7.0)}, and T_{lag(7.2)} of the oral buprenorphine dosage form is more than about 0.25, 0.5, 0.75, 1, 1.25, 1.5, 1.75, 2, 2.25, 2.5, 2.75, 3, 3.5, or 4 hours, preferably more than about 2, 2.25, 2.5, 2.75, or 3 hours.

In some embodiments, where the oral dosage form of the invention incorporates controlled release material, the T_{lag(5.0)}, T_{lag(5.5)}, T_{lag(6.8)} T_{lag(7.0)}, and T_{lag(7.2)} of the oral buprenorphine dosage form is more than about 0.25, 0.5, 0.75, 1, 1.25, 1.5, 1.75, 2, 2.25, 2.5, 2.75, 3, 3.5, or 4 hours, preferably more than about 2, 2.25, 2.5, 2.75, or 3 hours.

In some embodiments, where the oral dosage form of the invention incorporates controlled release material, the T_{lag(7.2)} of the oral buprenorphine dosage form is more than about 0.25, 0.5, 0.75, 1, 1.25, 1.5, 1.75, 2, 2.25, 2.5, 2.75, 3, 3.5, or 4 hours, preferably more than about 2, 2.25, 2.5, 2.75, or 3 hours.

In some preferred embodiments, the dosage form provides an oral pharmaceutical composition comprising a therapeutically effective amount of buprenorphine, said dosage form releasing or delivering buprenorphine at a controlled rate of release of about 0.05 mg/hr, or 0.1 mg/hr, or 0.2 mg/hr, or 0.3 mg/hr, or 0.4 mg/hr, or 0.5 mg/hr, or 0.6 mg/hr, or 0.7 mg/hr, or 0.8 mg/hr, or 0.9 mg/hr, or 1 mg/hr, or 1.1 mg/hr, or 1.2 mg/hr, or 1.3 mg/hr, or 1.4 mg/hr, or 1.5 mg/hr, or 1.6 mg/hr, or 1.7 mg/hr, or 1.8 mg/hr, or 2 mg/hr, or 2.2 mg/hr, or 2.3 mg/hr, or 2.4 mg/hr, or 2.5 mg/hr, or 2.6 mg/hr, or 2.7 mg/hr, or 2.8 mg/hr, or 2.9 mg/hr, or 3 mg/hr, or 3.4 mg/hr, or 3.5 mg/hr, or 3.6 mg/hr, or 3.7 mg/hr, or 3.8 mg/hr, or 3.9 mg/hr, or 4 mg/hr, or 4.2 mg/hr, or 4.4 mg/hr, or 4.6 mg/hr, or 4.8 mg/hr, or 5 mg/hr, or 5.5 mg/hr, or 6 mg/hr, or 6.5 mg/hr, or 7 mg/hr, or 7.5 mg/hr, or 8 mg/hr, or 8.5 mg/hr, or 9 mg/hr, or 9.5 mg/hr, or 10 mg/hr, or 11 mg/hr, or 12 mg/hr, or 14 mg/hr, or 16 mg/hr, or 18 mg/hr, or 20 mg/hr, or 25 mg/hr, or 30 mg/hr, or 35 mg/hr, or 40 mg/hr, or 50 mg/hr.

In some preferred embodiments, the dosage form provides an oral pharmaceutical composition comprising a therapeutically effective amount of buprenorphine and controlled release material to render said dosage form suitable for extended release in a human patient, said dosage form releasing or delivering buprenorphine at a controlled rate of release of about 0.1 mg/hr, or 0.2 mg/hr, or 0.3 mg/hr, or 0.4 mg/hr, or 0.5 mg/hr, or 0.6 mg/hr, or 0.7 mg/hr, or 0.8 mg/hr, or 0.9 mg/hr, or 1 mg/hr, or 1.1 mg/hr, or 1.2 mg/hr, or 1.3 mg/hr, or 1.4 mg/hr, or 1.5 mg/hr, or 1.6 mg/hr, or 1.7 mg/hr, or 1.8 mg/hr, or 2 mg/hr, or 2.2 mg/hr, or 2.3 mg/hr, or 2.4 mg/hr, or 2.5 mg/hr, or 2.6 mg/hr, or 2.7 mg/hr, or 2.8 mg/hr, or 2.9 mg/hr, or 3 mg/hr, or 3.4 mg/hr, or 3.5 mg/hr, or 3.6 mg/hr, or 3.7 mg/hr, or 3.8 mg/hr, or 3.9 mg/hr, or 4 mg/hr, or 4.2 mg/hr, or 4.4 mg/hr, or 4.6 mg/hr, or 4.8 mg/hr, or 5 mg/hr, or 6 mg/hr, or 7 mg/hr, or 8 mg/hr, or 9 mg/hr, or 10 mg/hr, more preferably at a rate of about 0.2 mg/hr, or 0.3 mg/hr, or 0.4 mg/hr, or 0.5 mg/hr, or 0.6 mg/hr, or 0.7 mg/hr, or 0.8 mg/hr, or 0.9 mg/hr, or 1 mg/hr, or 1.1 mg/hr, or 1.2 mg/hr, or 1.3 mg/hr, or 1.4 mg/hr, or 1.5 mg/hr, or 1.6 mg/hr, or 1.7 mg/hr, or 1.8 mg/hr, or 2 mg/hr, or 2.2 mg/hr, or 2.3 mg/hr, or 2.4 mg/hr, or 2.5 mg/hr, or 2.6 mg/hr, or 2.7 mg/hr, or 2.8 mg/hr, or 2.9 mg/hr, or 3 mg/hr.

In some preferred embodiments where the oral buprenorphine dosage form incorporates a controlled release material to render said dosage form suitable for extended release in a human patient, said dosage form releases or delivers buprenorphine at a controlled rate of release of 0.22 mg/hr to 2.5 mg/hr, more preferably, 0.22 mg/hr to 1.8 mg/hr, or 0.22 mg/hr to 1.7 mg/hr.

In some preferred embodiments, the dosage form provides an oral pharmaceutical composition comprising a therapeutically effective amount of buprenorphine, said dosage form releasing or delivering buprenorphine at a controlled rate of release of about 0.1 mg/hr to about 8 mg/hr, or about 0.2 mg/hr to about 8 mg/hr, or about 0.3 mg/hr to about 8 mg/hr, or about 0.4 mg/hr to about 8 mg/hr, or 0.5 mg/hr to about 8 mg/hr, or 0.6 mg/hr to about 8 mg/hr, or about 0.7 mg/hr to about 8 mg/hr, or about 0.8 mg/hr to about 8 mg/hr, or about 0.9 mg/hr to about 8 mg/hr, or about 1 mg/hr to about 8 mg/hr, or about 1.2 mg/hr to about 8 mg/hr, or about 1.4 mg/hr to about 8 mg/hr, or 1.5 mg/hr to about 8 mg/hr, or 1.6 mg/hr to about 8 mg/hr, or about 1.8 mg/hr to about 8 mg/hr, or about 2 mg/hr to about 8 mg/hr.

In some preferred embodiments, the dosage form provides an oral pharmaceutical composition comprising a therapeutically effective amount of buprenorphine and controlled release material to render said dosage form suitable for extended release in a human patient, said dosage form releasing or delivering buprenorphine at a controlled rate of release of about 0.1 mg/hr to about 8 mg/hr, or about 0.2 mg/hr to about 8 mg/hr, or about 0.3 mg/hr to about 8 mg/hr, or about 0.4 mg/hr to about 8 mg/hr, or 0.5 mg/hr to about 8 mg/hr, or 0.6 mg/hr to about 8 mg/hr, or about 0.7 mg/hr to about 8 mg/hr, or about 0.8 mg/hr to about 8 mg/hr, or about 0.9 mg/hr to about 8 mg/hr, or about 1 mg/hr to about 8 mg/hr, or about 1.2 mg/hr to about 8 mg/hr, or about 1.4 mg/hr to about 8 mg/hr, or 1.5 mg/hr to about 8 mg/hr, or 1.6 mg/hr to about 8 mg/hr, or about 1.8 mg/hr to about 8 mg/hr, or about 2 mg/hr to about 8 mg/hr.

In some preferred embodiments, the dosage form provides an oral pharmaceutical composition comprising a therapeutically effective amount of buprenorphine and controlled release material to render said dosage form suitable for extended release in a human patient, said dosage form releasing or delivering buprenorphine at a controlled rate of release of about 0.1 mg/hr to about 6 mg/hr, or about 0.2 mg/hr to about 6 mg/hr, or about 0.3 mg/hr to about 6 mg/hr, or about 0.4 mg/hr to about 6 mg/hr, or 0.5 mg/hr to about 6 mg/hr, or 0.6 mg/hr to about 6 mg/hr, or about 0.7 mg/hr to about 6 mg/hr, or about 0.8 mg/hr to about 6 mg/hr, or about 0.9 mg/hr to about 6 mg/hr, or about 1 mg/hr to about 6 mg/hr, or about 1.2 mg/hr to about 6 mg/hr, or about 1.4 mg/hr to about 6 mg/hr, or 1.5 mg/hr to about 6 mg/hr, or 1.6 mg/hr to about 6 mg/hr, or about 1.8 mg/hr to about 6 mg/hr, or about 2 mg/hr to about 6 mg/hr.

In some preferred embodiments, the dosage form provides an oral pharmaceutical composition comprising a therapeutically effective amount of buprenorphine and controlled release material to render said dosage form suitable for extended release in a human patient, said dosage form releasing or delivering buprenorphine at a controlled rate of release of about 0.1 mg/hr to about 4 mg/hr, or about 0.2 mg/hr to about 4 mg/hr, or about 0.3 mg/hr to about 4 mg/hr, or about 0.4 mg/hr to about 4 mg/hr, or 0.5 mg/hr to about 4 mg/hr, or 0.6 mg/hr to about 4 mg/hr, or about 0.7 mg/hr to about 4 mg/hr, or about 0.8 mg/hr to about 4 mg/hr, or about 0.9 mg/hr to about 4 mg/hr, or about 1 mg/hr to about 4 mg/hr, or about 1.2 mg/hr to about 4 mg/hr, or about 1.4 mg/hr to about 4 mg/hr, or 1.5 mg/hr to about 4 mg/hr, or 1.6 mg/hr to about 4 mg/hr, or about 1.8 mg/hr to about 4 mg/hr, or about 2 mg/hr to about 4 mg/hr.

In some preferred embodiments, the dosage form provides an oral pharmaceutical composition comprising a therapeutically effective amount of buprenorphine and controlled release material to render said dosage form suitable for extended release in a human patient, said dosage form releasing or delivering buprenorphine at a controlled rate of release of about 0.1 mg/hr to about 3 mg/hr, or about 0.2 mg/hr to about 3 mg/hr, or about 0.3 mg/hr to about 3 mg/hr, or about 0.4 mg/hr to about 3 mg/hr, or 0.5 mg/hr to about 3 mg/hr, or 0.6 mg/hr to about 3 mg/hr, or about 0.7 mg/hr to about 3 mg/hr, or about 0.8 mg/hr to about 3 mg/hr, or about 0.9 mg/hr to about 3 mg/hr, or about 1 mg/hr to about 3 mg/hr, or about 1.2 mg/hr to about 3 mg/hr, or about 1.4 mg/hr to about 3 mg/hr, or 1.5 mg/hr to about 3 mg/hr, or 1.6 mg/hr to about 3 mg/hr, or about 1.8 mg/hr to about 3 mg/hr, or about 2 mg/hr to about 3 mg/hr.

In some preferred embodiments, the dosage form provides an oral pharmaceutical composition comprising a therapeutically effective amount of buprenorphine and controlled release material to render said dosage form suitable for extended release in a human patient, said dosage form releasing or delivering buprenorphine at a controlled rate of release of about 0.1 mg/hr to about 4 mg/hr, or about 0.2 mg/hr to about 4 mg/hr, or about 0.3 mg/hr to about 4 mg/hr, or about 0.4 mg/hr to about 4 mg/hr, or 0.5 mg/hr to about 4 mg/hr, or 0.6 mg/hr to about 4 mg/hr, or about 0.7 mg/hr to about 4 mg/hr, or about 0.8 mg/hr to about 4 mg/hr, or about 0.9 mg/hr to about 4 mg/hr, or about 1 mg/hr to about 4 mg/hr, or about 1.2 mg/hr to about 4 mg/hr, or about 1.4 mg/hr to about 4 mg/hr, or 1.5 mg/hr to about 4 mg/hr, or 1.6 mg/hr to about 4 mg/hr, or about 1.8 mg/hr to about 4 mg/hr, or about 2 mg/hr to about 4 mg/hr.

In some preferred embodiments where the oral buprenorphine dosage form incorporates a controlled release material to render it as extended release suitable for dosing every 6 hours, said dosage form releases or delivers buprenorphine at a controlled rate of release for a period of about 2, 3, 4, 5, 6, 7, 8, 9, or 10 hours.

In some preferred embodiments where the oral buprenorphine dosage form incorporates a controlled release material to render it as extended release suitable for dosing every 8 hours, said dosage form releases or delivers buprenorphine at a controlled rate of release for a period of about 4, 5, 6, 7, 8, 9, 10 or 12 hours.

In some preferred embodiments where the oral buprenorphine dosage form incorporates a controlled release material to render it as extended release suitable for dosing every 12 hours, said dosage form releases or delivers buprenorphine at a controlled rate of release for a period of about 7, 8, 9, 10, 11, 12, 14, 16 or 18 hours.

In some preferred embodiments where the oral buprenorphine dosage form incorporates a controlled release material to render it as extended release suitable for dosing every 24 hours, said dosage form releases or delivers buprenorphine at a controlled rate of release for a period of about 12, 14, 16, 18, 20, 22, 24, 26 or 30 hours.

In some preferred embodiments where the oral buprenorphine dosage form incorporates a controlled release material to render it delayed onset (e.g., release or delivery of drug distal to the stomach, duodenum, or ileum) and where said delayed onset dosage form is intended to provide rapid release or burst release upon reaching the target GI anatomic location or GI environment, or at a desired time after oral ingestion (e.g., 2 to 6 hours), the dosage form releases or delivers buprenorphine in less than about 15, 30, 60, 90, 120, 160, 180 or 240 minutes, preferably in less than about 15, 30, 60, 90, or 120 minutes.

As used herein, “controlled rate of release” refers to the release or delivery of the active drug from the oral dosage form of the invention at rate per unit time over an extended period of time, or any time period over thirty-minutes up to thirty hours.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose of about 10 mg to about 20 mg for about 4 to about 10 days, then about 22 mg to about 40 mg for about 4 to about 10 days, and then about 50 mg to 600 mg for at least 1 day and optionally thereafter; or a dose of about 5 mg to about 20 mg for about 2 to about 7 days, then about 22 mg to about 60 mg for at least 1 day and optionally thereafter; or a dose of about 2 mg to about 20 mg for about 1 to about 7 days, then about 5 mg to about 40 mg for at least 1 day and optionally thereafter; or a dose of about 10 mg to about 20 mg for about 2 to about 7 days, then about 22 mg to about 40 mg for about 2 to about 7 days, and then about 50 mg to 600 mg for at least 1 day and optionally thereafter; or a dose of about 10 mg to about 15 mg for about 4 to about 10 days, then about 18 mg to about 40 mg for about 4 to about 10 days, and then about 50 mg to 600 mg for at least 1 day and optionally thereafter; or a dose of about 10 mg to about 15 mg for about 2 to about 7 days, then about 18 mg to about 40 mg for about 2 to about 7 days, and then about 50 mg to 600 mg for at least 1 day and optionally thereafter; or a dose of about 5 mg to about 8 mg for about 4 to about 10 days, then about 10 mg to about 20 mg for about 4 to about 10 days, and then about 25 mg to 600 mg for at least 1 day and optionally thereafter; or a dose of about 5 mg to about 8 mg for about 2 to about 7 days, then about 10 mg to about 20 mg for about 2 to about 7 days, and then about 25 mg to 600 mg for at least 1 day and optionally thereafter; or a dose of about 5 mg to about 8 mg for about 2 to about 7 days, then about 15 mg to about 20 mg for about 2 to about 7 days, and then about 25 mg to 600 mg for at least 1 day and optionally thereafter; or a dose of about 5 mg to about 10 mg for about 2 to about 7 days, then about 12 mg to about 20 mg for about 2 to about 7 days, and then about 25 mg to 600 mg for at least 1 day and optionally thereafter; or a dose of about 5 mg to about 10 mg for about 4 to about 10 days, then about 12 mg to about 20 mg for about 4 to about 10 days, and then about 25 mg to 600 mg for at least 1 day and optionally thereafter; or a dose of about 5 mg to about 10 mg for about 4 to about 10 days, then about 15 mg to about 20 mg for about 4 to about 10 days, and then about 25 mg to 600 mg for at least 1 day and optionally thereafter; or a dose of about 5 mg for about 4 to about 10 days, then about 10 mg for about 4 to about 10 days, and then about 15 mg to 600 mg for at least 1 day and optionally thereafter; or a dose of about 10 mg for about 4 to about 10 days, then about 15 mg for about 4 to about 10 days, and then about 20 mg to 600 mg for at least 1 day and optionally thereafter; or a dose of about 10 mg for about 4 to about 10 days, then about 20 mg for about 4 to about 10 days, and then about 25 mg to 600 mg for at least 1 day and optionally thereafter; or a dose of about 5 mg to about 10 mg for about 2 to about 7 days, then about 12 mg to about 20 mg for about 2 to about 7 days, and then about 25 mg to 600 mg for at least 1 day and optionally thereafter; or a dose of about 5 mg to about 10 mg for about 2 to about 7 days, then about 15 mg to about 20 mg for about 2 to about 7 days, and then about 25 mg to 600 mg for at least 1 day and optionally thereafter; or a dose of about 5 mg for about 2 to about 7 days, then about 10 mg for about 2 to about 7 days, and then about 15 mg to 600 mg for at least 1 day and optionally thereafter; or a dose of about 10 mg for about 2 to about 7 days, then about 15 mg for about 2 to about 7 days, and then about 20 mg to 600 mg for at least 1 day and optionally thereafter; or a dose of about 10 mg for about 2 to about 7 days, then about 20 mg for about 2 to about 7 days, and then about 25 mg to 600 mg for at least 1 day and optionally thereafter; or a dose of about 5 mg for about 4 to about 10 days, and then about 10 mg to 600 mg for at least 1 day and optionally thereafter; or a dose of about 10 mg for about 4 to about 10 days, and then about 15 mg to 600 mg for at least 1 day and optionally thereafter; or a dose of about 15 mg for about 4 to about 10 days, and then about 20 mg to 600 mg for at least 1 day and optionally thereafter; or a dose of about 20 mg for about 4 to about 10 days, and then about 25 mg to 600 mg for at least 1 day and optionally thereafter; or a dose of about 5 mg for about 2 to about 7 days, and then about 10 mg to 600 mg for at least 1 day and optionally thereafter; or a dose of about 10 mg for about 2 to about 7 days, and then about 15 mg to 600 mg for at least 1 day and optionally thereafter; or a dose of about 15 mg for about 2 to about 7 days, and then about 20 mg to 600 mg for at least 1 day and optionally thereafter; or a dose of about 20 mg for about 2 to about 7 days, and then about 25 mg to 600 mg for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said regimen comprising administering a dose of about 1 mg to about 5 mg for about 4 to about 10 days, then about 6 mg to about 10 mg for about 4 to about 10 days, and then about 12 mg to 200 for at least 1 day and optionally thereafter; or a dose of about 5 mg to about 20 mg for about 2 to about 7 days, then about 22 mg to about 60 mg for at least 1 day and optionally thereafter; or a dose of about 2 mg to about 20 mg for about 1 to about 7 days, then about 5 mg to about 40 mg for at least 1 day and optionally thereafter; or a dose of about 2 mg to about 8 mg for about 2 to about 7 days, then about 10 mg to about 15 mg for about 2 to about 7 days, and then about 16 mg to 200 for at least 1 day and optionally thereafter; or a dose of about 2 mg to about 5 mg for about 2 to about 7 days, then about 6 mg to about 10 mg for about 2 to about 7 days, and then about 12 mg to 200 for at least 1 day and optionally thereafter; or about 5 mg to about 8 mg for about 4 to about 10 days, then about 10 mg to about 20 mg for about 4 to about 10 days, and then about 25 mg to 200 for at least 1 day and optionally thereafter; or a dose of about 5 mg to about 8 mg for about 2 to about 7 days, then about 10 mg to about 20 mg for about 2 to about 7 days, and then about 25 mg to 200 for at least 1 day and optionally thereafter; or a dose of about 5 mg to about 8 mg for about 2 to about 7 days, then about 15 mg to about 20 mg for about 2 to about 7 days, and then about 25 mg to 200 for at least 1 day and optionally thereafter; or a dose of about 5 mg to about 10 mg for about 2 to about 7 days, then about 12 mg to about 20 mg for about 2 to about 7 days, and then about 25 mg to 200 for at least 1 day and optionally thereafter; or a dose of about 5 mg to about 10 mg for about 4 to about 10 days, then about 12 mg to about 20 mg for about 4 to about 10 days, and then about 25 mg to 200 for at least 1 day and optionally thereafter; or a dose of about 5 mg to about 10 mg for about 4 to about 10 days, then about 15 mg to about 20 mg for about 4 to about 10 days, and then about 25 mg to 200 for at least 1 day and optionally thereafter; or a dose of about 5 mg for about 4 to about 10 days, then about 10 mg for about 4 to about 10 days, and then about 15 mg to 200 for at least 1 day and optionally thereafter; or a dose of about 10 mg for about 4 to about 10 days, then about 15 mg for about 4 to about 10 days, and then about 20 mg to 200 for at least 1 day and optionally thereafter; or a dose of about 10 mg for about 4 to about 10 days, then about 20 mg for about 4 to about 10 days, and then about 25 mg to 200 for at least 1 day and optionally thereafter; or a dose of about 5 mg to about 10 mg for about 2 to about 7 days, then about 12 mg to about 20 mg for about 2 to about 7 days, and then about 25 mg to 200 for at least 1 day and optionally thereafter; or a dose of about 5 mg to about 10 mg for about 2 to about 7 days, then about 15 mg to about 20 mg for about 2 to about 7 days, and then about 25 mg to 200 for at least 1 day and optionally thereafter; or a dose of about 5 mg for about 2 to about 7 days, then about 10 mg for about 2 to about 7 days, and then about 15 mg to 200 for at least 1 day and optionally thereafter; or a dose of about 10 mg for about 2 to about 7 days, then about 15 mg for about 2 to about 7 days, and then about 20 mg to 200 for at least 1 day and optionally thereafter; or a dose of about 10 mg for about 2 to about 7 days, then about 20 mg for about 2 to about 7 days, and then about 25 mg to 200 for at least 1 day and optionally thereafter; or a dose of about 5 mg for about 4 to about 10 days, and then about 10 mg to 200 for at least 1 day and optionally thereafter; or a dose of about 10 mg for about 4 to about 10 days, and then about 15 mg to 200 for at least 1 day and optionally thereafter; or a dose of about 15 mg for about 4 to about 10 days, and then about 20 mg to 200 for at least 1 day and optionally thereafter; or a dose of about 20 mg for about 4 to about 10 days, and then about 25 mg to 200 for at least 1 day and optionally thereafter; or a dose of about 5 mg for about 2 to about 7 days, and then about 10 mg to 200 for at least 1 day and optionally thereafter; or a dose of about 10 mg for about 2 to about 7 days, and then about 15 mg to 200 for at least 1 day and optionally thereafter; or a dose of about 15 mg for about 2 to about 7 days, and then about 20 mg to 200 for at least 1 day and optionally thereafter; or a dose of about 20 mg for about 2 to about 7 days, and then about 25 mg to 200 for at least 1 day and optionally thereafter.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said regimen comprising administering a dose of about 1 mg to about 5 mg for about 2 to about 4 days, then about 6 mg to about 10 mg for about 2 to about 4 days, and then about 12 mg to 200 for at least 1 day and optionally thereafter; or a dose of about 2 mg to about 8 mg for about 1 to about 4 days, then about 10 mg to about 15 mg for about 1 to about 4 days, and then about 16 mg to 200 for at least 1 day and optionally thereafter; or a dose of about 2 mg to about 5 mg for about 1 to about 4 days, then about 6 mg to about 10 mg for about 1 to about 4 days, and then about 12 mg to 200 for at least 1 day and optionally thereafter; or about 5 mg to about 8 mg for about 2 to about 4 days, then about 10 mg to about 20 mg for about 2 to about 4 days, and then about 25 mg to 200 for at least 1 day and optionally thereafter; or a dose of about 5 mg to about 8 mg for about 1 to about 4 days, then about 10 mg to about 20 mg for about 1 to about 4 days, and then about 25 mg to 200 for at least 1 day and optionally thereafter; or a dose of about 5 mg to about 8 mg for about 1 to about 4 days, then about 15 mg to about 20 mg for about 1 to about 4 days, and then about 25 mg to 200 for at least 1 day and optionally thereafter; or a dose of about 5 mg to about 10 mg for about 1 to about 4 days, then about 12 mg to about 20 mg for about 1 to about 4 days, and then about 25 mg to 200 for at least 1 day and optionally thereafter; or a dose of about 5 mg to about 10 mg for about 2 to about 4 days, then about 12 mg to about 20 mg for about 2 to about 4 days, and then about 25 mg to 200 for at least 1 day and optionally thereafter; or a dose of about 5 mg to about 10 mg for about 2 to about 4 days, then about 15 mg to about 20 mg for about 2 to about 4 days, and then about 25 mg to 200 for at least 1 day and optionally thereafter; or a dose of about 5 mg for about 2 to about 4 days, then about 10 mg for about 2 to about 4 days, and then about 15 mg to 200 for at least 1 day and optionally thereafter; or a dose of about 10 mg for about 2 to about 4 days, then about 15 mg for about 2 to about 4 days, and then about 20 mg to 200 for at least 1 day and optionally thereafter; or a dose of about 10 mg for about 2 to about 4 days, then about 20 mg for about 2 to about 4 days, and then about 25 mg to 200 for at least 1 day and optionally thereafter; or a dose of about 5 mg to about 10 mg for about 1 to about 4 days, then about 12 mg to about 20 mg for about 1 to about 4 days, and then about 25 mg to 200 for at least 1 day and optionally thereafter; or a dose of about 5 mg to about 10 mg for about 1 to about 4 days, then about 15 mg to about 20 mg for about 1 to about 4 days, and then about 25 mg to 200 for at least 1 day and optionally thereafter; or a dose of about 5 mg for about 1 to about 4 days, then about 10 mg for about 1 to about 4 days, and then about 15 mg to 200 for at least 1 day and optionally thereafter; or a dose of about 10 mg for about 1 to about 4 days, then about 15 mg for about 1 to about 4 days, and then about 20 mg to 200 for at least 1 day and optionally thereafter; or a dose of about 10 mg for about 1 to about 4 days, then about 20 mg for about 1 to about 4 days, and then about 25 mg to 200 for at least 1 day and optionally thereafter; or a dose of about 5 mg for about 2 to about 4 days, and then about 10 mg to 200 for at least 1 day and optionally thereafter; or a dose of about 10 mg for about 2 to about 4 days, and then about 15 mg to 200 for at least 1 day and optionally thereafter; or a dose of about 15 mg for about 2 to about 4 days, and then about 20 mg to 200 for at least 1 day and optionally thereafter; or a dose of about 20 mg for about 2 to about 4 days, and then about 25 mg to 200 for at least 1 day and optionally thereafter; or a dose of about 5 mg for about 1 to about 4 days, and then about 10 mg to 200 for at least 1 day and optionally thereafter; or a dose of about 10 mg for about 1 to about 4 days, and then about 15 mg to 200 for at least 1 day and optionally thereafter; or a dose of about 15 mg for about 1 to about 4 days, and then about 20 mg to 200 for at least 1 day and optionally thereafter; or a dose of about 20 mg for about 1 to about 4 days, and then about 25 mg to 200 for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a total daily dose of a dose of about 10 mg to about 20 mg for about 4 to about 10 days, then about 22 mg to about 40 mg for about 4 to about 10 days, and then about 50 mg to 600 mg for at least 1 day and optionally thereafter; or a dose of about 5 mg to about 20 mg for about 2 to about 7 days, then about 22 mg to about 60 mg for at least 1 day and optionally thereafter; or a dose of about 10 mg to about 20 mg for about 2 to about 7 days, then about 22 mg to about 40 mg for about 2 to about 7 days, and then about 50 mg to 600 mg for at least 1 day and optionally thereafter; or a dose of about 10 mg to about 15 mg for about 4 to about 10 days, then about 18 mg to about 40 mg for about 4 to about 10 days, and then about 50 mg to 600 mg for at least 1 day and optionally thereafter; or a dose of about 10 mg to about 15 mg for about 2 to about 7 days, then about 18 mg to about 40 mg for about 2 to about 7 days, and then about 50 mg to 600 mg for at least 1 day and optionally thereafter; or about 5 mg to about 8 mg for about 4 to about 10 days, then about 10 mg to about 20 mg for about 4 to about 10 days, and then about 25 mg to 600 mg for at least 1 day and optionally thereafter; or a dose of about 5 mg to about 8 mg for about 2 to about 7 days, then about 10 mg to about 20 mg for about 2 to about 7 days, and then about 25 mg to 600 mg for at least 1 day and optionally thereafter; or a dose of about 5 mg to about 8 mg for about 2 to about 7 days, then about 15 mg to about 20 mg for about 2 to about 7 days, and then about 25 mg to 600 mg for at least 1 day and optionally thereafter; or a dose of about 5 mg to about 10 mg for about 2 to about 7 days, then about 12 mg to about 20 mg for about 2 to about 7 days, and then about 25 mg to 600 mg for at least 1 day and optionally thereafter; or a dose of about 5 mg to about 10 mg for about 4 to about 10 days, then about 12 mg to about 20 mg for about 4 to about 10 days, and then about 25 mg to 600 mg for at least 1 day and optionally thereafter; or a dose of about 5 mg to about 10 mg for about 4 to about 10 days, then about 15 mg to about 20 mg for about 4 to about 10 days, and then about 25 mg to 600 mg for at least 1 day and optionally thereafter; or a dose of about 5 mg for about 4 to about 10 days, then about 10 mg for about 4 to about 10 days, and then about 15 mg to 600 mg for at least 1 day and optionally thereafter; or a dose of about 10 mg for about 4 to about 10 days, then about 15 mg for about 4 to about 10 days, and then about 20 mg to 600 mg for at least 1 day and optionally thereafter; or a dose of about 10 mg for about 4 to about 10 days, then about 20 mg for about 4 to about 10 days, and then about 25 mg to 600 mg for at least 1 day and optionally thereafter; or a dose of about 5 mg to about 10 mg for about 2 to about 7 days, then about 12 mg to about 20 mg for about 2 to about 7 days, and then about 25 mg to 600 mg for at least 1 day and optionally thereafter; or a dose of about 5 mg to about 10 mg for about 2 to about 7 days, then about 15 mg to about 20 mg for about 2 to about 7 days, and then about 25 mg to 600 mg for at least 1 day and optionally thereafter; or a dose of about 5 mg for about 2 to about 7 days, then about 10 mg for about 2 to about 7 days, and then about 15 mg to 600 mg for at least 1 day and optionally thereafter; or a dose of about 10 mg for about 2 to about 7 days, then about 15 mg for about 2 to about 7 days, and then about 20 mg to 600 mg for at least 1 day and optionally thereafter; or a dose of about 10 mg for about 2 to about 7 days, then about 20 mg for about 2 to about 7 days, and then about 25 mg to 600 mg for at least 1 day and optionally thereafter; or a dose of about 5 mg for about 4 to about 10 days, and then about 10 mg to 600 mg for at least 1 day and optionally thereafter; or a dose of about 10 mg for about 4 to about 10 days, and then about 15 mg to 600 mg for at least 1 day and optionally thereafter; or a dose of about 15 mg for about 4 to about 10 days, and then about 20 mg to 600 mg for at least 1 day and optionally thereafter; or a dose of about 20 mg for about 4 to about 10 days, and then about 25 mg to 600 mg for at least 1 day and optionally thereafter; or a dose of about 5 mg for about 2 to about 7 days, and then about 10 mg to 600 mg for at least 1 day and optionally thereafter; or a dose of about 10 mg for about 2 to about 7 days, and then about 15 mg to 600 mg for at least 1 day and optionally thereafter; or a dose of about 15 mg for about 2 to about 7 days, and then about 20 mg to 600 mg for at least 1 day and optionally thereafter; or a dose of about 20 mg for about 2 to about 7 days, and then about 25 mg to 600 mg for at least 1 day and optionally thereafter; said total daily dose administered anywhere from anywhere from about six times-a-day (Q4H or Q4H PRN) to once-a-day (QD or QD PRN). In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said regimen comprising administering a total daily dose of about 1 mg to about 5 mg for about 4 to about 10 days, then about 6 mg to about 10 mg for about 4 to about 10 days, and then about 12 mg to 200 for at least 1 day and optionally thereafter; or a dose of about 5 mg to about 20 mg for about 2 to about 7 days, then about 22 mg to about 60 mg for at least 1 day and optionally thereafter; or a dose of about 2 mg to about 8 mg for about 2 to about 7 days, then about 10 mg to about 15 mg for about 2 to about 7 days, and then about 16 mg to 200 for at least 1 day and optionally thereafter; or a dose of about 2 mg to about 5 mg for about 2 to about 7 days, then about 6 mg to about 10 mg for about 2 to about 7 days, and then about 12 mg to 200 for at least 1 day and optionally thereafter; or about 5 mg to about 8 mg for about 4 to about 10 days, then about 10 mg to about 20 mg for about 4 to about 10 days, and then about 25 mg to 200 for at least 1 day and optionally thereafter; or a dose of about 5 mg to about 8 mg for about 2 to about 7 days, then about 10 mg to about 20 mg for about 2 to about 7 days, and then about 25 mg to 200 for at least 1 day and optionally thereafter; or a dose of about 5 mg to about 8 mg for about 2 to about 7 days, then about 15 mg to about 20 mg for about 2 to about 7 days, and then about 25 mg to 200 for at least 1 day and optionally thereafter; or a dose of about 5 mg to about 10 mg for about 2 to about 7 days, then about 12 mg to about 20 mg for about 2 to about 7 days, and then about 25 mg to 200 for at least 1 day and optionally thereafter; or a dose of about 5 mg to about 10 mg for about 4 to about 10 days, then about 12 mg to about 20 mg for about 4 to about 10 days, and then about 25 mg to 200 for at least 1 day and optionally thereafter; or a dose of about 5 mg to about 10 mg for about 4 to about 10 days, then about 15 mg to about 20 mg for about 4 to about 10 days, and then about 25 mg to 200 for at least 1 day and optionally thereafter; or a dose of about 5 mg for about 4 to about 10 days, then about 10 mg for about 4 to about 10 days, and then about 15 mg to 200 for at least 1 day and optionally thereafter; or a dose of about 10 mg for about 4 to about 10 days, then about 15 mg for about 4 to about 10 days, and then about 20 mg to 200 for at least 1 day and optionally thereafter; or a dose of about 10 mg for about 4 to about 10 days, then about 20 mg for about 4 to about 10 days, and then about 25 mg to 200 for at least 1 day and optionally thereafter; or a dose of about 5 mg to about 10 mg for about 2 to about 7 days, then about 12 mg to about 20 mg for about 2 to about 7 days, and then about 25 mg to 200 for at least 1 day and optionally thereafter; or a dose of about 5 mg to about 10 mg for about 2 to about 7 days, then about 15 mg to about 20 mg for about 2 to about 7 days, and then about 25 mg to 200 for at least 1 day and optionally thereafter; or a dose of about 5 mg for about 2 to about 7 days, then about 10 mg for about 2 to about 7 days, and then about 15 mg to 200 for at least 1 day and optionally thereafter; or a dose of about 10 mg for about 2 to about 7 days, then about 15 mg for about 2 to about 7 days, and then about 20 mg to 200 for at least 1 day and optionally thereafter; or a dose of about 10 mg for about 2 to about 7 days, then about 20 mg for about 2 to about 7 days, and then about 25 mg to 200 for at least 1 day and optionally thereafter; or a dose of about 5 mg for about 4 to about 10 days, and then about 10 mg to 200 for at least 1 day and optionally thereafter; or a dose of about 10 mg for about 4 to about 10 days, and then about 15 mg to 200 for at least 1 day and optionally thereafter; or a dose of about 15 mg for about 4 to about 10 days, and then about 20 mg to 200 for at least 1 day and optionally thereafter; or a dose of about 20 mg for about 4 to about 10 days, and then about 25 mg to 200 for at least 1 day and optionally thereafter; or a dose of about 5 mg for about 2 to about 7 days, and then about 10 mg to 200 for at least 1 day and optionally thereafter; or a dose of about 10 mg for about 2 to about 7 days, and then about 15 mg to 200 for at least 1 day and optionally thereafter; or a dose of about 15 mg for about 2 to about 7 days, and then about 20 mg to 200 for at least 1 day and optionally thereafter; or a dose of about 20 mg for about 2 to about 7 days, and then about 25 mg to 200 for at least 1 day and optionally thereafter; said total daily dose administered anywhere from about six times-a-day (Q4H or Q4H PRN) to once-a-day (QD or QD PRN).

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said regimen comprising administering a total daily dose of about 1 mg to about 5 mg for about 1 to about 4 days, then about 6 mg to about 10 mg for about 1 to about 4 days, and then about 12 mg to 200 for at least 1 day and optionally thereafter; or a dose of about 2 mg to about 8 mg for about 1 to about 4 days, then about 10 mg to about 15 mg for about 1 to about 4 days, and then about 16 mg to 200 for at least 1 day and optionally thereafter; or a dose of about 2 mg to about 5 mg for about 1 to about 4 days, then about 6 mg to about 10 mg for about 1 to about 4 days, and then about 12 mg to 200 for at least 1 day and optionally thereafter; or about 5 mg to about 8 mg for about 1 to about 4 days, then about 10 mg to about 20 mg for about 1 to about 4 days, and then about 25 mg to 200 for at least 1 day and optionally thereafter; or a dose of about 5 mg to about 8 mg for about 1 to about 4 days, then about 10 mg to about 20 mg for about 1 to about 4 days, and then about 25 mg to 200 for at least 1 day and optionally thereafter; or a dose of about 5 mg to about 8 mg for about 1 to about 4 days, then about 15 mg to about 20 mg for about 1 to about 4 days, and then about 25 mg to 200 for at least 1 day and optionally thereafter; or a dose of about 5 mg to about 10 mg for about 1 to about 4 days, then about 12 mg to about 20 mg for about 1 to about 4 days, and then about 25 mg to 200 for at least 1 day and optionally thereafter; or a dose of about 5 mg to about 10 mg for about 1 to about 4 days, then about 12 mg to about 20 mg for about 1 to about 4 days, and then about 25 mg to 200 for at least 1 day and optionally thereafter; or a dose of about 5 mg to about 10 mg for about 1 to about 4 days, then about 15 mg to about 20 mg for about 1 to about 4 days, and then about 25 mg to 200 for at least 1 day and optionally thereafter; or a dose of about 5 mg for about 1 to about 4 days, then about 10 mg for about 1 to about 4 days, and then about 15 mg to 200 for at least 1 day and optionally thereafter; or a dose of about 10 mg for about 1 to about 4 days, then about 15 mg for about 1 to about 4 days, and then about 20 mg to 200 for at least 1 day and optionally thereafter; or a dose of about 10 mg for about 1 to about 4 days, then about 20 mg for about 1 to about 4 days, and then about 25 mg to 200 for at least 1 day and optionally thereafter; or a dose of about 5 mg to about 10 mg for about 1 to about 4 days, then about 12 mg to about 20 mg for about 1 to about 4 days, and then about 25 mg to 200 for at least 1 day and optionally thereafter; or a dose of about 5 mg to about 10 mg for about 1 to about 4 days, then about 15 mg to about 20 mg for about 1 to about 4 days, and then about 25 mg to 200 for at least 1 day and optionally thereafter; or a dose of about 5 mg for about 1 to about 4 days, then about 10 mg for about 1 to about 4 days, and then about 15 mg to 200 for at least 1 day and optionally thereafter; or a dose of about 10 mg for about 1 to about 4 days, then about 15 mg for about 1 to about 4 days, and then about 20 mg to 200 for at least 1 day and optionally thereafter; or a dose of about 10 mg for about 1 to about 4 days, then about 20 mg for about 1 to about 4 days, and then about 25 mg to 200 for at least 1 day and optionally thereafter; or a dose of about 5 mg for about 1 to about 4 days, and then about 10 mg to 200 for at least 1 day and optionally thereafter; or a dose of about 10 mg for about 1 to about 4 days, and then about 15 mg to 200 for at least 1 day and optionally thereafter; or a dose of about 15 mg for about 1 to about 4 days, and then about 20 mg to 200 for at least 1 day and optionally thereafter; or a dose of about 20 mg for about 1 to about 4 days, and then about 25 mg to 200 for at least 1 day and optionally thereafter; or a dose of about 5 mg for about 1 to about 4 days, and then about 10 mg to 200 for at least 1 day and optionally thereafter; or a dose of about 10 mg for about 1 to about 4 days, and then about 15 mg to 200 for at least 1 day and optionally thereafter; or a dose of about 15 mg for about 1 to about 4 days, and then about 20 mg to 200 for at least 1 day and optionally thereafter; or a dose of about 20 mg for about 1 to about 4 days, and then about 25 mg to 200 for at least 1 day and optionally thereafter; said total daily dose administered anywhere from about six times-a-day (Q4H or Q4H PRN) to once-a-day (QD or QD PRN).

In some preferred embodiments, the dosage form provides an oral pharmaceutical composition comprising a therapeutically effective amount of buprenorphine; optionally; said dosage form intended to treat pediatric patients; said dosage form administered at a prespecified dosing regimen; said dosing regimen providing a mean buprenorphine and norbuprenorphine area under the plasma concentration time curve (AUC) as provided herein, except that the AUC is multiplied by the ratio obtained from the child's weight in kilograms divided by 70 kilograms.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to 24 hours post-dose (AUC₀₋₂₄) of about 1000 pg·hr/mL to about 15000 pg·hr/mL for about 1 to about 10 days, then about 5000 pg·hr/mL to about 40000 pg·hr/mL for at least 1 day and optionally thereafter; or about 5000 pg·hr/mL to about 20000 pg·hr/mL for about 1 to about 10 days, then about 7500 pg·hr/mL to about 80000 pg·hr/mL for at least 1 day and optionally thereafter; or about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to 24 hours post-dose (AUC₀₋₂₄) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC₀₋₂₄ of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC₀₋₂₄ of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC₀₋₂₄ of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC₀₋₂₄ of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC₀₋₂₄ of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean buprenorphine area under the plasma concentration time curve to infinity (AUC_{0-inf}) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 4 to about 10 days, then about 2500 pg·hr/mL to about 7500 pg·hr/mL for about 4 to about 10 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 4 to about 10 days, then about 5000 pg·hr/mL to about 12500 pg·hr/mL for about 4 to about 10 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 4 to about 10 days, then about 7500 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 15000 pg·hr/mL for about 4 to about 10 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 4 to about 10 days, then about 10000 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 4 to about 10 days, then about 7500 pg·hr/mL to about 20000 pg·hr/mL for about 4 to about 10 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 4 to about 10 days, then about 10000 pg·hr/mL to about 40000 pg·hr/mL for about 4 to about 10 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 4 to about 10 days, then about 2500 pg·hr/mL to about 40000 pg·hr/mL for about 4 to about 10 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 2 to about 7 days, then about 2500 pg·hr/mL to about 7500 pg·hr/mL for about 2 to about 7 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 2 to about 7 days, then about 5000 pg·hr/mL to about 12500 pg·hr/mL for about 2 to about 7 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 2 to about 7 days, then about 7500 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 15000 pg·hr/mL for about 2 to about 7 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 2 to about 7 days, then about 10000 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 2 to about 7 days, then about 7500 pg·hr/mL to about 20000 pg·hr/mL for about 2 to about 7 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 2 to about 7 days, then about 10000 pg·hr/mL to about 40000 pg·hr/mL for about 2 to about 7 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 2 to about 7 days, then about 2500 pg·hr/mL to about 40000 pg·hr/mL for about 2 to about 7 days, and then about 10000 pg·hr/mL to about 40000 pg·hr/mL for at least 1 day and optionally thereafter.

In some preferred embodiments, the dosage form provides an oral pharmaceutical composition comprising a therapeutically effective amount of buprenorphine; an oral controlled release material to render said dosage form suitable for extended release, or delayed onset, extended release or delayed onset, rapid release or delayed onset, pulsatile release human patient; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter. In some embodiments, the oral controlled release material is optional.

In some preferred embodiments, the dosage form provides an oral pharmaceutical composition comprising a therapeutically effective amount of buprenorphine; said dosage form suitable for immediate release in a human patient; said dosage form administered at a prespecified dosing regimen; said dosage form administered at a prespecified dosing regimen; said regimen comprising administering a dose which provides a mean norbuprenorphine area under the plasma concentration time curve to infinity (AUC_{0-inf}) of about 1000 pg·hr/mL to about 5000 pg·hr/mL for about 1 to about 4 days, then about 2500 pg·hr/mL to about 7500 pg·hr/mL for about 1 to about 4 days, and then about 5000 pg·hr/mL to about 500,000 pg·hr/mL for at least 1 day and optionally thereafter; or an AUC_{0-inf} of about 2500 pg·hr/mL to about 7500 pg·hr/mL for about 1 to about 4 days, then about 5000 pg·hr/mL to about 12500 pg·hr/mL for about 1 to about 4 days, and then about 7500 pg·hr/mL to about 500,000 pg·hr/mL for at least 1 day and optionally thereafter; or an AUC_{0-inf} of about 5000 pg·hr/mL to about 10000 pg·hr/mL for about 1 to about 4 days, then about 7500 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 15000 pg·hr/mL for about 1 to about 4 days, and then about 10000 pg·hr/mL to about 500,000 pg·hr/mL for at least 1 day and optionally thereafter; or an AUC_{0-inf} of about 5000 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 7500 pg·hr/mL to about 15000 pg·hr/mL for about 1 to about 4 days, then about 10000 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 20000 pg·hr/mL for about 1 to about 4 days, then about 7500 pg·hr/mL to about 20000 pg·hr/mL for about 1 to about 4 days, and then about 15000 pg·hr/mL to about 500,000 pg·hr/mL for at least 1 day and optionally thereafter; or an AUC_{0-inf} of about 5000 pg·hr/mL to about 30000 pg·hr/mL for about 1 to about 4 days, then about 10000 pg·hr/mL to about 40000 pg·hr/mL for about 1 to about 4 days, and then about 15000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 500,000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 300000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 200000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 175000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 150000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 125000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 100000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 80000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 60000 pg·hr/mL for at least 1 day and optionally thereafter; or an AUC_{0-inf} of about 1000 pg·hr/mL to about 20000 pg·hr/mL for about 1 to about 4 days, then about 2500 pg·hr/mL to about 40000 pg·hr/mL for about 1 to about 4 days, and then about 10000 pg·hr/mL to about 40000 pg·hr/mL for at least 1 day and optionally thereafter.

In a preferred embodiment, the dosage form containing buprenorphine or a pharmaceutically acceptable salt of buprenorphine or a mixture thereof is an extended release form. Oral, extended release buprenorphine has several distinct advantages over oral immediate release opioids and over sublingual, lingual and buccal dosage forms, including fewer interruptions in sleep, reduced dependence on caregivers, improved compliance, enhanced quality of life outcomes, and increased control over the management of their malady (e.g., pain). In addition, such formulations can provide more constant plasma concentrations and clinical effects, less frequent peak to trough fluctuations and fewer side effects.

In some preferred embodiments, the dosage form provides an oral pharmaceutical composition comprising a therapeutically effective amount of buprenorphine or a pharmaceutically acceptable salt of buprenorphine or a mixture thereof; optionally, a controlled release material to render said dosage form suitable for up to every 24 hour (once-a-day) administration to a human patient; said dosage form providing at least 10% of the steady state concentration of buprenorphine and norbuprenorphine after administration of one dose at its intended dosing frequency. In other preferred embodiments, the dosage form provides at least about 15%, or at least about 20%, or at least about 30%, or at least about 40%, or at least about 50%, or at least about 60%, or at least about 70%, or at least about 75%, or at least about 80%, or at least about 85%, or at least about 90% of the steady state therapeutic concentration of buprenorphine after administration of one dose or two doses at their intended dosing frequency.

In some preferred embodiments, the invention provides a method of providing relief in a human patient suffering from pain comprising a therapeutically effective amount of oral buprenorphine or a pharmaceutically acceptable salt of buprenorphine or a mixture thereof.

In some preferred embodiments, the invention provides a method of providing relief in a human patient suffering from cough, dyspnea, opioid addiction disorders, restless leg syndrome, acute herpes zoster, visceral pain, breakthrough pain, opioid dependence and urinary incontinence comprising a therapeutically effective amount of oral buprenorphine or a pharmaceutically acceptable salt of buprenorphine or a mixture thereof.

In some preferred embodiments, the invention provides a method of providing relief in a human patient suffering from a buprenorphine responsive medical condition comprising a therapeutically effective amount of oral buprenorphine or a pharmaceutically acceptable salt of buprenorphine or a mixture thereof.

In some preferred embodiments, the invention comprises an oral pharmaceutical composition comprising a therapeutically effective amount of buprenorphine and controlled release material to render said dosage form suitable for three times a day administration (or about every eight hours administration).

In some preferred embodiments, the oral pharmaceutical composition provides a therapeutic effect for about 1, 2, 3, 4, 5, or 6 hours.

In some preferred embodiments, the oral pharmaceutical composition is used on a time contingent basis.

In some preferred embodiments, the oral pharmaceutical composition is used on a pain contingent basis.

In some preferred embodiments, the QID or Q6H oral pharmaceutical composition provides a therapeutic effect for about 6 hours.

In some preferred embodiments, the TID or Q8H oral pharmaceutical composition provides a therapeutic effect for about 8 hours.

In some preferred embodiments, the Q4H or Q4H PRN oral pharmaceutical composition provides a Cₘₐₓ of buprenorphine and norbuprenorphine at about 1 to about 4 hours.

In some preferred embodiments, the Q4H or Q4H PRN oral pharmaceutical composition provides a Cₘᵢₙ of buprenorphine and norbuprenorphine at about 3 to 6 hours.

In some preferred embodiments, the Q4H or Q4H PRN oral pharmaceutical composition provides a mean buprenorphine AUC_{0-inf} after first administration or AUC₀₋₄ at steady state of about 50 pg·hr/mL to about 20,000 pg·hr/mL.

In some preferred embodiments, the Q4H or Q4H PRN oral pharmaceutical composition provides a mean of buprenorphine and norbuprenorphine C₄/Cmax ratio of 0.05 to about 1.25.

In some preferred embodiments, the Q4H or Q4H PRN oral pharmaceutical composition provides a buprenorphine and norbuprenorphine percent fluctuation of less than 400%.

In some preferred embodiments, the Q4H or Q4H PRN oral pharmaceutical composition provides of buprenorphine and norbuprenorphine W₅₀ of 0.5 to about 3.5 hours.

In some preferred embodiments, the Q4H or Q4H PRN oral pharmaceutical composition provides a HVD of buprenorphine and norbuprenorphine of 0.75 to about 3.75 hours.

In some preferred embodiments, the Q4H or Q4H PRN oral pharmaceutical composition provides an AI of buprenorphine and norbuprenorphine of not more than 4.0.

In some preferred embodiments, the Q6H or Q6H PRN oral pharmaceutical composition provides a Cₘₐₓ of buprenorphine and norbuprenorphine at about 1 to about 6 hours.

In some preferred embodiments, the Q6H or Q6H PRN oral pharmaceutical composition provides a Cₘᵢₙ of buprenorphine and norbuprenorphine at about 3 to 8 hours.

In some preferred embodiments, the Q6H or Q6H PRN oral pharmaceutical composition provides a mean buprenorphine AUC_{0-inf} after first administration or AUC₀₋₄ at steady state of about 50 pg·hr/mL to about 20,000 pg·hr/mL.

In some preferred embodiments, the Q6H or Q6H PRN oral pharmaceutical composition provides a mean of buprenorphine and norbuprenorphine C₄/Cmax ratio of 0.05 to about 1.25.

In some preferred embodiments, the Q6H or Q6H PRN oral pharmaceutical composition provides a buprenorphine and norbuprenorphine percent fluctuation of less than 400%.

In some preferred embodiments, the Q6H or Q6H PRN oral pharmaceutical composition provides of buprenorphine and norbuprenorphine W₅₀ of 1.0 to about 5 hours.

In some preferred embodiments, the Q6H or Q6H PRN oral pharmaceutical composition provides a HVD of buprenorphine and norbuprenorphine of 1.25 to about 5.5 hours.

In some preferred embodiments, the Q6H or Q6H PRN oral pharmaceutical composition provides an AI of buprenorphine and norbuprenorphine of not more than 4.0.

In some preferred embodiments, the TID or Q8H oral pharmaceutical composition provides a Cₘₐₓ of buprenorphine and norbuprenorphine at about 1 to about 6 hours.

In some preferred embodiments, the TID or Q8H oral pharmaceutical composition provides a Cₘᵢₙ of buprenorphine and norbuprenorphine at about 6 to 10 hours.

In some preferred embodiments, the TID or Q8H oral pharmaceutical composition provides a mean buprenorphine AUC_{0-inf} after first administration or AUC₀₋₈ at steady state of about 125 pg·hr/mL to about 150,000 pg·hr/mL, or about 125 pg·hr/mL to about 100,000 pg·hr/mL, or about 125 pg·hr/mL to about 75,000 pg·hr/mL, or about 125 pg·hr/mL to about 50,000 pg·hr/mL, or about 125 pg·hr/mL to about 30,000 pg·hr/mL, or about 125 pg·hr/mL to about 20,000 pg·hr/mL, or

In some preferred embodiments, the TID or Q8H oral pharmaceutical composition provides a mean of buprenorphine and norbuprenorphine C₈/Cmax ratio of 0.05 to about 1.25.

In some preferred embodiments, the TID or Q8H oral pharmaceutical composition provides an buprenorphine and norbuprenorphine percent fluctuation of less than 400%.

In some preferred embodiments, the TID or Q8H oral pharmaceutical composition provides of buprenorphine and norbuprenorphine W₅₀ of 1.5 to about 6.5 hours.

In some preferred embodiments, the TID or Q8H oral pharmaceutical composition provides a HVD of buprenorphine and norbuprenorphine of 2 to about 7 hours.

In some preferred embodiments, the TID or Q8H oral pharmaceutical composition provides an AI of buprenorphine and norbuprenorphine of not more than 4.0.

In some preferred embodiments, the invention comprises an oral pharmaceutical composition comprising a therapeutically effective amount of buprenorphine; said dosage from providing a Cₘₐₓ of buprenorphine and norbuprenorphine from about 200 pg/mL to about 40,000 pg/mL. In other preferred embodiments, the dosage form provides a Cₘₐₓ of buprenorphine and norbuprenorphine of about 400 pg/mL to about 40,000 pg/mL, or about 600 pg/mL to about 40,000 pg/mL or about 800 pg/mL to about 40,000 pg/mL, or about 1000 pg/mL to about 40,000 pg/mL or about 2000 pg/mL to about 40,000 pg/mL, or about 3000 pg/mL to about 40,000 pg/mL or about 4000 pg/mL to about 40,000 pg/mL or about 5000 pg/mL to about 40,000 pg/mL, or about 6000 pg/mL to about 40,000 pg/mL or about 7000 pg/mL to about 40,000 pg/mL, or about 8000 pg/mL to about 40,000 pg/mL or about 200 pg/mL to about 35,000 pg/mL or about 200 pg/mL to about 30,000 pg/mL, or about 200 pg/mL to about 25,000 pg/mL or about 200 pg/mL to about 20,000 pg/mL, or about 200 pg/mL to about 15,000 pg/mL or about 200 pg/mL to about 10,000 pg/mL or about 200 pg/mL to about 8,000 pg/mL, or about 200 pg/mL to about 7,000 pg/mL or about 200 pg/mL to about 6,000 pg/mL, or about 200 pg/mL to about 5,000 pg/mL.

In some preferred embodiments, the invention comprises an oral pharmaceutical composition comprising a therapeutically effective amount of buprenorphine; said dosage from providing a Cₘₐₓ of buprenorphine and/or norbuprenorphine from about 20 pg/mL to about 6000 pg/mL. In other preferred embodiments, the dosage form provides a Cₘₐₓ of buprenorphine and norbuprenorphine of about 40 pg/mL to about 6000 pg/mL, or about 50 pg/mL to about 6000 pg/mL, or about 75 pg/mL to about 6000 pg/mL, or about 100 pg/mL to about 6000 pg/mL, or about 125 pg/mL to about 6000 pg/mL, or about 150 pg/mL to about 6000 pg/mL, or about 175 pg/mL to about 6000 pg/mL, or about 200 pg/mL to about 6000 pg/mL, or about 225 pg/mL to about 6000 pg/mL, or about 250 pg/mL to about 6000 pg/mL, or about 300 pg/mL to about 6000 pg/mL, or about 350 pg/mL to about 6000 pg/mL, or about 400 pg/mL to about 6000 pg/mL, or about 450 pg/mL to about 6000 pg/mL, or about 500 pg/mL to about 6000 pg/mL, or about 600 pg/mL to about 6000 pg/mL, or about 700 pg/mL to about 6000 pg/mL, or about 800 pg/mL to about 6000 pg/mL, or about 1000 pg/mL to about 6000 pg/mL, or about 1200 pg/mL to about 6000 pg/mL, or about 50 pg/mL to about 5000 pg/mL, or about 100 pg/mL to about 5000 pg/mL, or about 50 pg/mL to about 4000 pg/mL, or about 100 pg/mL to about 4000 pg/mL, or about 200 pg/mL to about 4000 pg/mL, or about 50 pg/mL to about 3000 pg/mL, or about 100 pg/mL to about 3000 pg/mL, or about 50 pg/mL to about 2000 pg/mL, or about 100 pg/mL to about 2000 pg/mL, or about 200 pg/mL to about 2000 pg/mL, or about 50 pg/mL to about 1500 pg/mL, or about 100 pg/mL to about 1500 pg/mL, or about 50 pg/mL to about 1000 pg/mL, or about 100 pg/mL to about 1000 pg/mL, or about 50 pg/mL to about 800 pg/mL, or about 100 pg/mL to about 800 pg/mL, or about 20 pg/mL to about 5000 pg/mL, or about 20 pg/mL to about 4500 pg/mL, or about 20 pg/mL to about 4000 pg/mL, or about 20 pg/mL to about 3500 pg/mL, or about 20 pg/mL to about 3000 pg/mL, or about 20 pg/mL to about 2500 pg/mL, or about 20 pg/mL to about 2000 pg/mL, or about 20 pg/mL to about 1800 pg/mL, or about 20 pg/mL to about 1600 pg/mL, or about 20 pg/mL to about 1500 pg/mL, or about 20 pg/mL to about 1400 pg/mL, or about 20 pg/mL to about 1200 pg/mL, or about 20 pg/mL to about 1100 pg/mL, or about 20 pg/mL to about 1000 pg/mL, or about 20 pg/mL to about 900 pg/mL, or about 20 pg/mL to about 800 pg/mL, or about 20 pg/mL to about 700 pg/mL, or about 20 pg/mL to about 600 pg/mL, or about 20 pg/mL to about 500 pg/mL, or about 100 pg/mL to about 2000 pg/mL, or about 100 pg/mL to about 1800 pg/mL, or about 100 pg/mL to about 1600 pg/mL, or about 100 pg/mL to about 1500 pg/mL, or about 100 pg/mL to about 1400 pg/mL, or about 100 pg/mL to about 1200 pg/mL, or about 100 pg/mL to about 1100 pg/mL, or about 100 pg/mL to about 100 pg/mL, or about 1000 pg/mL to about 900 pg/mL, or about 100 pg/mL to about 800 pg/mL, or about 100 pg/mL to about 700 pg/mL, or about 100 pg/mL to about 600 pg/mL, or about 100 pg/mL to about 500 pg/mL, or about 200 pg/mL to about 1600 pg/mL, or about 200 pg/mL to about 1500 pg/mL, or about 200 pg/mL to about 1400 pg/mL, or about 200 pg/mL to about 1200 pg/mL, or about 200 pg/mL to about 1100 pg/mL, or about 200 pg/mL to about 1000 pg/mL, or about 200 pg/mL to about 900 pg/mL, or about 200 pg/mL to about 800 pg/mL, or about 200 pg/mL to about 700 pg/mL, or about 200 pg/mL to about 600 pg/mL, or about 200 pg/mL to about 500 pg/mL. In some preferred embodiments, the aforementioned Cₘₐₓ being achieved with oral pharmaceutical compositions comprising a controlled release material to render said dosage form extended release, or delayed onset, extended release, or delayed onset, rapid release, or delayed onset, pulsatile release.

In some preferred embodiments, the invention comprises an oral pharmaceutical composition comprising a therapeutically effective amount of buprenorphine; said dosage from providing a Cₘₐₓ of buprenorphine and norbuprenorphine occurring from a mean of about 0.25 to about 30 hours. In other preferred embodiments, the dosage form provides a Cₘₐₓ of buprenorphine and norbuprenorphine occurring from a mean of about 0.5 to about 30 hours, or from a mean of about 1 to about 30 hours, or about 1 to about 26 hours, or about 1 to about 24 hours, or about 1 to about 20 hours, or about 1 to about 18 hours, or about 1 to about 16 hours, or about 1 to about 14 hours, or about 1 to about 12 hours, or about 1 to about 10 hours, or about 1 to about 8 hours, or about 1 to about 6 hours, or about 1 to about 4 hours, or about 1 to about 3 hours, or about 2 to about 30 hours, or about 4 to about 30 hours, or about 4 to about 24 hours, or about 6 to about 24 hours, or about 8 to about 24 hours, or about 10 to about 20 hours, or about 12 to about 24 hours, or about 18 to about 24 hours, or about 2 to about 12 hours, or about 3 to about 12 hours, or about 3 to about 8 hours, or about 4 to about 10 hours, or about 4 to about 12 hours, or about 4 to about 9 hours, or about 5 to about 8 hours. In some preferred embodiments, the aforementioned time to Cₘₐₓ being achieved with oral pharmaceutical compositions comprising a controlled release material to render said dosage form extended release, or delayed onset, extended release, or delayed onset, rapid release, or delayed onset, pulsatile release.

In some preferred embodiments, the invention comprises an oral pharmaceutical composition comprising a therapeutically effective amount of buprenorphine; a controlled release material to render said dosage form suitable for extended release in a human patient.

In some preferred embodiments, the invention comprises an oral pharmaceutical composition comprising a therapeutically effective amount of buprenorphine; said dosage from providing a Cₘᵢₙ of buprenorphine and norbuprenorphine of about 0 pg/mL to about 20,000 pg/mL or 1 pg/mL to about 20,000 pg/mL. In other preferred embodiments, the dosage form provides a Cₘᵢₙ of buprenorphine and norbuprenorphine of less than about 20,000 pg/mL, or less than about 18,000 pg/mL, or less than about 16,000 pg/mL, or less than about 15,000 pg/mL, or less than about 14,000 pg/mL, or less than about 12,000 pg/mL, or less than about 10,000 pg/mL, or less than about 9,000 pg/mL, or less than about 8,000 pg/mL, or less than about 7,000 pg/mL, or less than about 6,000 pg/mL, or less than about 5,000 pg/mL, or less than about 4,000 pg/mL, or less than about 3,000 pg/mL, or less than about 2,000 pg/mL, or less than about 1,000 pg/mL,

In some preferred embodiments, the invention comprises an oral pharmaceutical composition comprising a therapeutically effective amount of buprenorphine; said dosage from providing a Cₘᵢₙ of buprenorphine and norbuprenorphine of about 0 pg/mL to about 3000 pg/mL or 1 pg/mL to about 3000 pg/mL. In other preferred embodiments, the dosage form provides a Cₘᵢₙ of buprenorphine and norbuprenorphine of less than about 2500 pg/mL, or less than about 2000 pg/mL, or less than about 1800 pg/mL, or less than about 1600 pg/mL, or less than about 1800 pg/mL, or less than about 1500 pg/mL, or less than about 1400 pg/mL, or less than about 1200 pg/mL, or less than about 1100 pg/mL, or less than about 1000 pg/mL, or less than about 900 pg/mL, or less than about 800 pg/mL, or less than about 700 pg/mL, or less than about 600 pg/mL, or less than about 500 pg/mL, or less than about 400 pg/mL, or less than about 300 pg/mL, or less than about 250 pg/mL, or less than about 200 pg/mL, or less than about 175 pg/mL, or less than about 150 pg/mL, or less than about 120 pg/mL, or less than about 100 pg/mL, or less than about 90 pg/mL, or less than about 80 pg/mL, or less than about 70 pg/mL, or less than about 60 pg/mL, or less than about 50 pg/mL, or less than about 40 pg/mL, or less than about 30 pg/mL, or less than about 20 pg/mL, or less than about 15 pg/mL, or less than about 10 pg/mL, or less than about 5 pg/mL, or less than about 2 pg/mL, or up to about 3000 pg/mL. In some preferred embodiments, the aforementioned Cₘᵢₙ being achieved with oral pharmaceutical compositions comprising a controlled release material to render said dosage form extended release, or delayed onset, extended release, or delayed onset, rapid release, or delayed onset, pulsatile release.

In some preferred embodiments, the invention comprises an oral pharmaceutical composition comprising a therapeutically effective amount of buprenorphine; said dosage from providing a Cₘᵢₙ of buprenorphine and norbuprenorphine measured from a mean of about 0.5 to about 30 hours. In other preferred embodiments, the dosage form provides a Cₘᵢₙ of buprenorphine and norbuprenorphine measured from a mean of about 0.5 to about 28 hours, or about 1 to about 28 hours, or about 1 to 24 hours, or about 1 to about 20 hours, or about 1 to about 18 hours, or about 1 to about 16 hours, or about 1 to about 12 hours, or about 1 to 10 hours, or about 1 to about 8 hours, or about 1 to about 6 hours, or about 1 to about 4 hours, about 2 to about 24 hours, or about 3 to 24 hours, or about 4 to about 24 hours, or about 6 to about 24 hours, or about 8 to about 24 hours, about 2 to about 12 hours, or about 3 to 10 hours, or about 3 to about 8 hours, or about 4 to about 8 hours, or about 6 to about 10 hours. In some preferred embodiments, the aforementioned Cₘᵢₙ being achieved with oral pharmaceutical compositions comprising a controlled release material to render said dosage form extended release, or delayed onset, extended release, or delayed onset, rapid release, or delayed onset, pulsatile release.

In some preferred embodiments, the invention comprises an oral pharmaceutical composition comprising a therapeutically effective amount of buprenorphine; said dosage form providing a systemic exposure as assessed by the mean of buprenorphine and norbuprenorphine area under the plasma concentration time curves to 24 hours post-dose (AUC₀₋₂₄) of about 500 pg·hr/mL to about 500,000 pg·hr/mL. In other preferred embodiments, the dosage form provides an AUC₀₋₂₄ of buprenorphine and norbuprenorphine of about 50 pg·hr/mL to about 475000 pg·hr/mL, or about 500 pg·hr/mL to about 450000 pg·hr/mL, or about 500 pg·hr/mL to about 425000 pg·hr/mL, or about 500 pg·hr/mL to about 400000 pg·hr/mL, or about 500 pg·hr/mL to about 375000 pg·hr/mL, or about 500 pg·hr/mL to about 350000 pg·hr/mL, or about 500 pg·hr/mL to about 325000 pg·hr/mL, or about 500 pg·hr/mL to about 300000 pg·hr/mL, or about 500 pg·hr/mL to about 275000 pg·hr/mL, or about 500 pg·hr/mL to about 250000 pg·hr/mL, or about 500 pg·hr/mL to about 225000 pg·hr/mL, or about 500 pg·hr/mL to about 200000 pg·hr/mL, or about 500 pg·hr/mL to about 175000 pg·hr/mL, or about 500 pg·hr/mL to about 150000 pg·hr/mL, or about 500 pg·hr/mL to about 125000 pg·hr/mL, or about 500 pg·hr/mL to about 100000 pg·hr/mL, or about 500 pg·hr/mL to about 75000 pg·hr/mL, or about 500 pg·hr/mL to about 50000 pg·hr/mL, or about 1000 pg·hr/mL to about 500000 pg·hr/mL, or about 1500 pg·hr/mL to about 500000 pg·hr/mL, or about 2000 pg·hr/mL to about 500000 pg·hr/mL, or about 3500 pg·hr/mL to about 500000 pg·hr/mL, or about 4000 pg·hr/mL to about 500000 pg·hr/mL, or about 5000 pg·hr/mL to about 500000 pg·hr/mL, or about 6000 pg·hr/mL to about 500000 pg·hr/mL, or about 8000 pg·hr/mL to about 500000 pg·hr/mL, or about 9000 pg·hr/mL to about 500000 pg·hr/mL, or about 10000 pg·hr/mL to about 500000 pg·hr/mL, or about 1000 pg·hr/mL to about 300000 pg·hr/mL, or about 3000 pg·hr/mL to about 300000 pg·hr/mL.

In some preferred embodiments, the invention comprises an oral pharmaceutical composition comprising a therapeutically effective amount of buprenorphine; said dosage form providing a systemic exposure as assessed by the mean of buprenorphine and norbuprenorphine area under the plasma concentration time curves to infinity (AUC_{0-inf}) of about 500 pg·hr/mL to about 500,000 pg·hr/mL. In other preferred embodiments, the dosage form provides an AUC_{0-inf} of buprenorphine and norbuprenorphine of about 50 pg·hr/mL to about 475000 pg·hr/mL, or about 500 pg·hr/mL to about 450000 pg·hr/mL, or about 500 pg·hr/mL to about 425000 pg·hr/mL, or about 500 pg·hr/mL to about 400000 pg·hr/mL, or about 500 pg·hr/mL to about 375000 pg·hr/mL, or about 500 pg·hr/mL to about 350000 pg·hr/mL, or about 500 pg·hr/mL to about 325000 pg·hr/mL, or about 500 pg·hr/mL to about 300000 pg·hr/mL, or about 500 pg·hr/mL to about 275000 pg·hr/mL, or about 500 pg·hr/mL to about 250000 pg·hr/mL, or about 500 pg·hr/mL to about 225000 pg·hr/mL, or about 500 pg·hr/mL to about 200000 pg·hr/mL, or about 500 pg·hr/mL to about 175000 pg·hr/mL, or about 500 pg·hr/mL to about 150000 pg·hr/mL, or about 500 pg·hr/mL to about 125000 pg·hr/mL, or about 500 pg·hr/mL to about 100000 pg·hr/mL, or about 500 pg·hr/mL to about 75000 pg·hr/mL, or about 500 pg·hr/mL to about 50000 pg·hr/mL, or about 1000 pg·hr/mL to about 500000 pg·hr/mL, or about 1500 pg·hr/mL to about 500000 pg·hr/mL, or about 2000 pg·hr/mL to about 500000 pg·hr/mL, or about 3500 pg·hr/mL to about 500000 pg·hr/mL, or about 4000 pg·hr/mL to about 500000 pg·hr/mL, or about 5000 pg·hr/mL to about 500000 pg·hr/mL, or about 6000 pg·hr/mL to about 500000 pg·hr/mL, or about 8000 pg·hr/mL to about 500000 pg·hr/mL, or about 9000 pg·hr/mL to about 500000 pg·hr/mL, or about 10000 pg·hr/mL to about 500000 pg·hr/mL, or about 1000 pg·hr/mL to about 300000 pg·hr/mL, or about 3000 pg·hr/mL to about 300000 pg·hr/mL.

In some preferred embodiments, the invention comprises an oral pharmaceutical composition comprising a therapeutically effective amount of buprenorphine; said dosage form providing a systemic exposure as assessed by the mean of buprenorphine and norbuprenorphine area under the plasma concentration time curves to 24 hours post-dose (AUC₀₋₂₄) of about 50 pg·hr/mL to about 80000 pg·hr/mL. In other preferred embodiments, the dosage form provides an AUC₀₋₂₄ of buprenorphine and norbuprenorphine of about 50 pg·hr/mL to about 70000 pg·hr/mL, or about 50 pg·hr/mL to about 60000 pg·hr/mL, or about 50 pg·hr/mL to about 50000 pg·hr/mL, or about 50 pg·hr/mL to about 40000 pg·hr/mL, or about 50 pg·hr/mL to about 30000 pg·hr/mL, or about 50 pg·hr/mL to about 25000 pg·hr/mL, or about 50 pg·hr/mL to about 20000 pg·hr/mL, or about 50 pg·hr/mL to about 18000 pg·hr/mL, or about 50 pg·hr/mL to about 15000 pg·hr/mL, or about 50 pg·hr/mL to about 14000 pg·hr/mL, or about 50 pg·hr/mL to about 12000 pg·hr/mL, or about 50 pg·hr/mL to about 11000 pg·hr/mL, or about 50 pg·hr/mL to about 10000 pg·hr/mL, or about 50 pg·hr/mL to about 9000 pg·hr/mL, 50 pg·hr/mL to about 8500 pg·hr/mL, or about 50 pg·hr/mL to about 7000 pg·hr/mL, or about 50 pg·hr/mL to about 5000 pg·hr/mL, or about 50 pg·hr/mL to about 3500 pg·hr/mL, or about 50 pg·hr/mL to about 2500 pg·hr/mL, 50 pg·hr/mL to about 1700 pg·hr/mL, or about 50 pg·hr/mL to about 1200 pg·hr/mL, or about 100 pg·hr/mL to about 40000 pg·hr/mL, or about 500 pg·hr/mL to about 20000 pg·hr/mL, or about 500 pg·hr/mL to about 10000 pg·hr/mL, or about 1000 pg·hr/mL to about 20000 pg·hr/mL, or about 1000 pg·hr/mL to about 10000 pg·hr/mL, or about 1000 pg·hr/mL to about 8000 pg·hr/mL, 2000 pg·hr/mL to about 20000 pg·hr/mL, or about 2000 pg·hr/mL to about 15000 pg·hr/mL, or about 2000 pg·hr/mL to about 10000 pg·hr/mL, or about 2000 pg·hr/mL to about 8000 pg·hr/mL, or about 3000 pg·hr/mL to about 40000 pg·hr/mL, or about 3000 pg·hr/mL to about 30000 pg·hr/mL, or about 3000 pg·hr/mL to about 20000 pg·hr/mL, or about 3000 pg·hr/mL to about 15000 pg·hr/mL, or about 4000 pg·hr/mL to about 40000 pg·hr/mL, or about 4000 pg·hr/mL to about 30000 pg·hr/mL, or about 4000 pg·hr/mL to about 20000 pg·hr/mL, or about 5000 pg·hr/mL to about 30000 pg·hr/mL, or about 5000 pg·hr/mL to about 20000 pg·hr/mL, or about 5000 pg·hr/mL to about 15000 pg·hr/mL, or about 6000 pg·hr/mL to about 80000 pg·hr/mL, or about 6000 pg·hr/mL to about 40000 pg·hr/mL, or about 6000 pg·hr/mL to about 30000 pg·hr/mL, or about 100 pg·hr/mL to about 80000 pg·hr/mL, about 200 pg·hr/mL to about 80000 pg·hr/mL, or about 300 pg·hr/mL to about 80000 pg·hr/mL, or about 500 pg·hr/mL to about 80000 pg·hr/mL, or about 700 pg·hr/mL to about 80000 pg·hr/mL, or about 800 pg·hr/mL to about 80000 pg·hr/mL, or about 1000 pg·hr/mL to about 80000 pg·hr/mL, or about 1200 pg·hr/mL to about 80000 pg·hr/mL, or about 1500 pg·hr/mL to about 80000 pg·hr/mL, or about 2000 pg·hr/mL to about 80000 pg·hr/mL, or about 2500 pg·hr/mL to about 80000 pg·hr/mL, or about 3000 pg·hr/mL to about 80000 pg·hr/mL, or about 3500 pg·hr/mL to about 80000 pg·hr/mL, or about 4000 pg·hr/mL to about 80000 pg·hr/mL, or about 5000 pg·hr/mL to about 80000 pg·hr/mL, 6000 pg·hr/mL to about 80000 pg·hr/mL, or about 8000 pg·hr/mL to about 80000 pg·hr/mL, or about 10000 pg·hr/mL to about 80000 pg·hr/mL, or about 12000 pg·hr/mL to about 80000 pg·hr/mL, or about 15000 pg·hr/mL to about 80000 pg·hr/mL, or about 100 pg·hr/mL to about 40000 pg·hr/mL, about 200 pg·hr/mL to about 40000 pg·hr/mL, or about 300 pg·hr/mL to about 40000 pg·hr/mL, or about 500 pg·hr/mL to about 40000 pg·hr/mL, or about 700 pg·hr/mL to about 40000 pg·hr/mL, or about 800 pg·hr/mL to about 40000 pg·hr/mL, or about 1000 pg·hr/mL to about 40000 pg·hr/mL, or about 1200 pg·hr/mL to about 40000 pg·hr/mL, or about 1500 pg·hr/mL to about 40000 pg·hr/mL, or about 2000 pg·hr/mL to about 40000 pg·hr/mL, or about 2500 pg·hr/mL to about 40000 pg·hr/mL, or about 3000 pg·hr/mL to about 40000 pg·hr/mL, or about 3500 pg·hr/mL to about 40000 pg·hr/mL, or about 4000 pg·hr/mL to about 40000 pg·hr/mL, or about 5000 pg·hr/mL to about 40000 pg·hr/mL, 6000 pg·hr/mL to about 40000 pg·hr/mL, or about 8000 pg·hr/mL to about 40000 pg·hr/mL, or about 10000 pg·hr/mL to about 40000 pg·hr/mL, or about 12000 pg·hr/mL to about 40000 pg·hr/mL, or about 15000 pg·hr/mL to about 40000 pg·hr/mL. In some preferred embodiments, the aforementioned AUC₀₋₂₄ being achieved with oral pharmaceutical compositions comprising a controlled release material to render said dosage form extended release, or delayed onset, extended release, or delayed onset, rapid release, or delayed onset, pulsatile release.

In some preferred embodiments, the invention comprises an oral pharmaceutical composition comprising a therapeutically effective amount of buprenorphine; said dosage form providing a systemic exposure as assessed by the mean buprenorphine and norbuprenorphine area under the plasma concentration time curve to infinity (AUC_{0-inf}) of about 50 pg·hr/mL to about 80000 pg·hr/mL. In other preferred embodiments, the dosage form provides an AUC_{0-inf} of buprenorphine and norbuprenorphine of about 50 pg·hr/mL to about 70000 pg·hr/mL, or about 50 pg·hr/mL to about 60000 pg·hr/mL, or about 50 pg·hr/mL to about 50000 pg·hr/mL, or about 50 pg·hr/mL to about 40000 pg·hr/mL, or about 50 pg·hr/mL to about 30000 pg·hr/mL, or about 50 pg·hr/mL to about 25000 pg·hr/mL, or about 50 pg·hr/mL to about 20000 pg·hr/mL, or about 50 pg·hr/mL to about 18000 pg·hr/mL, or about 50 pg·hr/mL to about 15000 pg·hr/mL, or about 50 pg·hr/mL to about 14000 pg·hr/mL, or about 50 pg·hr/mL to about 12000 pg·hr/mL, or about 50 pg·hr/mL to about 11000 pg·hr/mL, or about 50 pg·hr/mL to about 10000 pg·hr/mL, or about 50 pg·hr/mL to about 9000 pg·hr/mL, 50 pg·hr/mL to about 8500 pg·hr/mL, or about 50 pg·hr/mL to about 7000 pg·hr/mL, or about 50 pg·hr/mL to about 5000 pg·hr/mL, or about 50 pg·hr/mL to about 3500 pg·hr/mL, or about 50 pg·hr/mL to about 2500 pg·hr/mL, 50 pg·hr/mL to about 1700 pg·hr/mL, or about 50 pg·hr/mL to about 1200 pg·hr/mL, or about 100 pg·hr/mL to about 40000 pg·hr/mL, or about 500 pg·hr/mL to about 20000 pg·hr/mL, or about 500 pg·hr/mL to about 10000 pg·hr/mL, or about 1000 pg·hr/mL to about 20000 pg·hr/mL, or about 1000 pg·hr/mL to about 10000 pg·hr/mL, or about 1000 pg·hr/mL to about 8000 pg·hr/mL, 2000 pg·hr/mL to about 20000 pg·hr/mL, or about 2000 pg·hr/mL to about 15000 pg·hr/mL, or about 2000 pg·hr/mL to about 10000 pg·hr/mL, or about 2000 pg·hr/mL to about 8000 pg·hr/mL, or about 3000 pg·hr/mL to about 40000 pg·hr/mL, or about 3000 pg·hr/mL to about 30000 pg·hr/mL, or about 3000 pg·hr/mL to about 20000 pg·hr/mL, or about 3000 pg·hr/mL to about 15000 pg·hr/mL, or about 4000 pg·hr/mL to about 40000 pg·hr/mL, or about 4000 pg·hr/mL to about 30000 pg·hr/mL, or about 4000 pg·hr/mL to about 20000 pg·hr/mL, or about 5000 pg·hr/mL to about 30000 pg·hr/mL, or about 5000 pg·hr/mL to about 20000 pg·hr/mL, or about 5000 pg·hr/mL to about 15000 pg·hr/mL, or about 6000 pg·hr/mL to about 80000 pg·hr/mL, or about 6000 pg·hr/mL to about 40000 pg·hr/mL, or about 6000 pg·hr/mL to about 30000 pg·hr/mL, or about 100 pg·hr/mL to about 80000 pg·hr/mL, about 200 pg·hr/mL to about 80000 pg·hr/mL, or about 300 pg·hr/mL to about 80000 pg·hr/mL, or about 500 pg·hr/mL to about 80000 pg·hr/mL, or about 700 pg·hr/mL to about 80000 pg·hr/mL, or about 800 pg·hr/mL to about 80000 pg·hr/mL, or about 1000 pg·hr/mL to about 80000 pg·hr/mL, or about 1200 pg·hr/mL to about 80000 pg·hr/mL, or about 1500 pg·hr/mL to about 80000 pg·hr/mL, or about 2000 pg·hr/mL to about 80000 pg·hr/mL, or about 2500 pg·hr/mL to about 80000 pg·hr/mL, or about 3000 pg·hr/mL to about 80000 pg·hr/mL, or about 3500 pg·hr/mL to about 80000 pg·hr/mL, or about 4000 pg·hr/mL to about 80000 pg·hr/mL, or about 5000 pg·hr/mL to about 80000 pg·hr/mL, 6000 pg·hr/mL to about 80000 pg·hr/mL, or about 8000 pg·hr/mL to about 80000 pg·hr/mL, or about 10000 pg·hr/mL to about 80000 pg·hr/mL, or about 12000 pg·hr/mL to about 80000 pg·hr/mL, or about 15000 pg·hr/mL to about 80000 pg·hr/mL, or about 100 pg·hr/mL to about 40000 pg·hr/mL, about 200 pg·hr/mL to about 40000 pg·hr/mL, or about 300 pg·hr/mL to about 40000 pg·hr/mL, or about 500 pg·hr/mL to about 40000 pg·hr/mL, or about 700 pg·hr/mL to about 40000 pg·hr/mL, or about 800 pg·hr/mL to about 40000 pg·hr/mL, or about 1000 pg·hr/mL to about 40000 pg·hr/mL, or about 1200 pg·hr/mL to about 40000 pg·hr/mL, or about 1500 pg·hr/mL to about 40000 pg·hr/mL, or about 2000 pg·hr/mL to about 40000 pg·hr/mL, or about 2500 pg·hr/mL to about 40000 pg·hr/mL, or about 3000 pg·hr/mL to about 40000 pg·hr/mL, or about 3500 pg·hr/mL to about 40000 pg·hr/mL, or about 4000 pg·hr/mL to about 40000 pg·hr/mL, or about 5000 pg·hr/mL to about 40000 pg·hr/mL, 6000 pg·hr/mL to about 40000 pg·hr/mL, or about 8000 pg·hr/mL to about 40000 pg·hr/mL, or about 10000 pg·hr/mL to about 40000 pg·hr/mL, or about 12000 pg·hr/mL to about 40000 pg·hr/mL, or about 15000 pg·hr/mL to about 40000 pg·hr/mL. In some preferred embodiments, the aforementioned AUC_{0-inf} being achieved with oral pharmaceutical compositions comprising a controlled release material to render said dosage form extended release, or delayed onset, extended release, or delayed onset, rapid release, or delayed onset, pulsatile release.

In some preferred embodiments, the dosage form provides an oral pharmaceutical composition comprising a therapeutically effective amount of buprenorphine; said dosage form providing a systemic exposure as assessed by the mean buprenorphine and norbuprenorphine area under the plasma concentration time curve (AUC₀₋₂₄) of up to 80000 pg·hr/mL, or up to 70000 pg·hr/mL, or up to 60000 pg·hr/mL, or up to 50000 pg·hr/mL, or up to 40000 pg·hr/mL, or up to 38000 pg·hr/mL, or up to 35000 pg·hr/mL, or up to 33000 pg·hr/mL, or up to 31000 pg·hr/mL, or up to 30000 pg·hr/mL, or up to 29000 pg·hr/mL, or up to 28000 pg·hr/mL, or up to 26000 pg·hr/mL, or up to 25000 pg·hr/mL, up to 23000 pg·hr/mL, or up to 22000 pg·hr/mL, or up to 21000 pg·hr/mL, or up to 20000 pg·hr/mL, or up to 18000 pg·hr/mL, or up to 17000 pg·hr/mL, or up to 15000 pg·hr/mL, or up to 14000 pg·hr/mL, or up to 12000 pg·hr/mL, or up to 10000 pg·hr/mL.

In some preferred embodiments, the dosage form provides an oral pharmaceutical composition comprising a therapeutically effective amount of buprenorphine; said dosage form providing at least 80% of the steady state therapeutic concentration of buprenorphine and norbuprenorphine after administration of ≦three doses at their intended dosing frequency.

In some preferred embodiments, the dosage form provides an oral pharmaceutical composition comprising a therapeutically effective amount of buprenorphine; said dosage form after administration to a human patient providing a Cₘᵢₙ/Cₘₐₓ ratio of buprenorphine and norbuprenorphine of 0.1 to about 1.25. In other preferred embodiments, the dosage form provides a Cₘᵢₙ/Cₘₐₓ ratio of buprenorphine and norbuprenorphine of about 0.1 to about 1.15, about 0.1 to about 1.19, about 0.1 to about 1, about 0.1 to about 0.9, about 0.1 to about 0.85, or about 0.1 to about 0.8, or about 0.1 to about 0.7, or about 0.1 to about 0.6, or about 0.1 to about 0.5, or about 0.1 to about 0.4, or about 0.1 to about 0.3, or about 0.2 to about 1.0, or about 0.25 to about 1.25, or about 0.25 to about 1.25, or about 0.4 to about 1.25, or about 0.5 to about 1.25, or about 0.65 to about 1.25, or about 0.75 to about 1.25, or about 0.2 to about 0.9, or about 0.3 to about 0.9, or about 0.3 to about 0.8, or about 0.4 to about 0.8, or about 0.4 to about 0.7, or about 0.4 to about 0.6.

In some preferred embodiments, the dosage form provides an oral pharmaceutical composition comprising a therapeutically effective amount of buprenorphine; said dosage form after administration to a human patient providing a percent fluctuation of buprenorphine and norbuprenorphine of less than 400%. In other preferred embodiments, the dosage form provides a percent fluctuation of buprenorphine and norbuprenorphine of less than 350%, or less than 300%, or less than 250%, or less than 200%, or less than 150%, or less than 100%, or less than 75%, or less than 50%, or less than 25%.

In some preferred embodiments, the dosage form provides an oral pharmaceutical composition comprising a therapeutically effective amount of buprenorphine; said dosage form after administration to a human patient providing a W₅₀ of buprenorphine and norbuprenorphine of about 1 to about 6 hours for each 6 hour time period of intended dosing frequency and intended duration of action. In other preferred embodiments, the dosage form provides a W₅₀ of buprenorphine and norbuprenorphine for each 6 hour time period of intended dosing frequency and intended duration of action of about 1 to about 5 hours, or about 1 to about 4 hours, or about 1 to about 3 hours, or about 1 to about 2 hours, or 2 to about 6 hours, or about 3 to about 6 hours, or about 4 to about 6 hours, or about 2 to about 4 hours.

In some preferred embodiments, the dosage form provides an oral pharmaceutical composition comprising a therapeutically effective amount of buprenorphine; said dosage form after administration to a human patient providing a HVD of buprenorphine and norbuprenorphine of about 1.5 to about 6 hours for each 6 hour time period of intended dosing frequency and intended duration of action. In other preferred embodiments, the dosage form provides a HVD of buprenorphine and norbuprenorphine for each 6 hour time period of intended dosing frequency and intended duration of action of about 1.5 to about 5 hours, or about 1.5 to about 4 hours, or about 1.5 to about 3 hours, or about 1.5 to about 2 hours, or 2 to about 6 hours, or about 3 to about 6 hours, or about 4 to about 6 hours, or about 2 to about 4 hours.

In some preferred embodiments, the dosage form provides an oral pharmaceutical composition comprising a therapeutically effective amount of buprenorphine; said dosage form after administration to a human patient providing an AI of buprenorphine and norbuprenorphine of not more than 4.0. In other preferred embodiments, the dosage form provides an AI of buprenorphine and norbuprenorphine of not more than about 3.5, or not more than about 3.0, or not more than about 2.5, or not more than about 2, or not more than about 1.75, or not more than about 1.5, or not more than about 1.25, or not more than about 1, or not more than about 0.75, or not more than about 0.5, or not more than about 0.25.

In some preferred embodiments, the in vivo pharmacokinetic parameters of the specifications, embodiments and claims are achieved with oral pharmaceutical compositions comprising a controlled release material to render said dosage form suitable for extended release.

In some preferred embodiments, the specifications and claims are achieved with oral pharmaceutical compositions comprising a controlled release material to render said dosage form suitable for extended release.

In some preferred embodiments, the dosage form provides an oral pharmaceutical composition comprising a therapeutically effective amount of buprenorphine; and a controlled release material with gastroretentive properties to render said dosage form suitable for extended release oral administration to a human patient.

In some preferred embodiments, the dosage form provides an oral pharmaceutical composition comprising a therapeutically effective amount of buprenorphine; and a controlled release material with osmotic release to render said dosage form suitable for extended release oral administration to a human patient.

In some preferred embodiments, the dosage form provides an oral pharmaceutical composition comprising a therapeutically effective amount of buprenorphine; and a controlled release material with zero-order or pseudo-zero-order release to render said dosage form suitable for extended release oral administration to a human patient.

In some preferred embodiments, the dosage form provides an oral pharmaceutical composition comprising a therapeutically effective amount of buprenorphine and a controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a percent fluctuation of buprenorphine and norbuprenorphine of less than 400%. In other preferred embodiments, the dosage form provides a percent fluctuation of buprenorphine and norbuprenorphine of less than about 375%, or less than about 350%, or less than about 325%, or less than about 300%, or less than about 275%, or less than about 250%, or less than about 225%, or less than about 200%, or less than about 175%, or less than about 150%, or less than about 125%, or less than about 100%, or less than about 75%, or less than about 50%, or less than about 25%. In some preferred embodiments, the aforementioned percent fluctuation being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient, providing an AI of buprenorphine and norbuprenorphine of not more than 4.0. In other preferred embodiments, the dosage form provides an AI of buprenorphine and norbuprenorphine of not more than about 3.75, or not more than about 3.5, or not more than about 3.25, or not more than about 3, or not more than about 2.75, or not more than about 2.5, or not more than about 2, or not more than about 1.5, not more than about 1.25, or not more than about 1, or not more than about 0.75. In some preferred embodiments, the aforementioned AI ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a mean buprenorphine and norbuprenorphine Cₘₐₓ of about 20 pg/mL to about 2000 pg/mL. In other preferred embodiments, the dosage form provides a mean buprenorphine and norbuprenorphine Cₘₐₓ of about 20 pg/mL to 1800 pg/mL, or about 20 pg/mL to 1600 pg/mL, or about 20 pg/mL to 1500 pg/mL, or about 20 pg/mL to 1400 pg/mL, or about 20 pg/mL to 1200 pg/mL, or about 20 pg/mL to 1100 pg/mL, or about 20 pg/mL to 1000 pg/mL, or about 20 pg/mL to 900 pg/mL, or about 20 pg/mL to 800 pg/mL, or about 20 pg/mL to 700 pg/mL, or about 20 pg/mL to 600 pg/mL, or about 20 pg/mL to 500 pg/mL, or about 20 pg/mL to 400 pg/mL, or about 20 pg/mL to 300 pg/mL, or about 20 pg/mL to 200 pg/mL, or about 50 pg/mL to 1800 pg/mL, or about 50 pg/mL to 1600 pg/mL, or about 50 pg/mL to 1500 pg/mL, or about 50 pg/mL to 1400 pg/mL, or about 50 pg/mL to 1500 pg/mL, or about 50 pg/mL to 1100 pg/mL, or about 50 pg/mL to 1000 pg/mL, or about 50 pg/mL to 900 pg/mL, or about 50 pg/mL to 800 pg/mL, or about 50 pg/mL to 700 pg/mL, or about 50 pg/mL to 600 pg/mL, or about 50 pg/mL to 500 pg/mL, or about 50 pg/mL to 400 pg/mL, or about 50 pg/mL to 300 pg/mL, or about 100 pg/mL to 1800 pg/mL, or about 100 pg/mL to 1600 pg/mL, or about 100 pg/mL to 11000 pg/mL, or about 100 pg/mL to 1400 pg/mL, or about 100 pg/mL to 1200 pg/mL, or about 100 pg/mL to 1100 pg/mL, or about 100 pg/mL to 1000 pg/mL, or about 100 pg/mL to 900 pg/mL, or about 100 pg/mL to 800 pg/mL, or about 100 pg/mL to 700 pg/mL, or about 100 pg/mL to 600 pg/mL, or about 100 pg/mL to 1000 pg/mL, or about 100 pg/mL to 400 pg/mL, or about 100 pg/mL to 300 pg/mL, or about 100 pg/mL to 1000 pg/mL, or about 200 pg/mL to 1600 pg/mL, or about 200 pg/mL to 1600 pg/mL, or about 200 pg/mL to 12000 pg/mL, or about 200 pg/mL to 1400 pg/mL, or about 200 pg/mL to 12000 pg/mL, or about 200 pg/mL to 1200 pg/mL, or about 200 pg/mL to 2000 pg/mL, or about 200 pg/mL to 900 pg/mL, or about 200 pg/mL to 800 pg/mL, or about 200 pg/mL to 700 pg/mL, or about 200 pg/mL to 600 pg/mL, or about 200 pg/mL to 2000 pg/mL, or about 200 pg/mL to 400 pg/mL, or about 200 pg/mL to 300 pg/mL, or about 200 pg/mL to 2000 pg/mL. In some preferred embodiments, the aforementioned Cₘₐₓ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a mean buprenorphine and norbuprenorphine Cmax of less than about 4000 pg/mL, or less than about 3000 pg/mL, or less than about 2000 pg/mL. In other preferred embodiments, the dosage form provides a mean buprenorphine and norbuprenorphine Cₘₐₓ of less than about 1800 pg/mL, or less than about 1700 pg/mL, or less than about 1600 pg/mL, or less than about 1500 pg/mL, or less than about 1400 pg/mL, or less than about 1200 pg/mL, or less than about 100 pg/mL, or less than about 900 pg/mL, or less than about 800 pg/mL, or less than about 700 pg/mL, or less than about 600 pg/mL, or less than about 500 pg/mL, or less than about 400 pg/mL, or less than about 300 pg/mL, or less than about 200 pg/mL, or less than about 100 pg/mL, or less than about 50 pg/mL. In some preferred embodiments, the aforementioned Cₘₐₓ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a mean buprenorphine and norbuprenorphine Cₘᵢₙ of about 1 pg/mL to about 1000 pg/mL. In other preferred embodiments, the dosage form provides a mean buprenorphine and norbuprenorphine Cₘᵢₙ of about 1 pg/mL to 950 pg/mL, or about 1 pg/mL to 900 pg/mL, or about 1 pg/mL to 850 pg/mL, or about 1 pg/mL to 800 pg/mL, or about 1 pg/mL to 750 pg/mL, or about 1 pg/mL to 700 pg/mL, or about 1 pg/mL to 650 pg/mL, or about 1 pg/mL to 600 pg/mL, or about 1 pg/mL to 550 pg/mL, or about 1 pg/mL to 500 pg/mL, or about 1 pg/mL to 450 pg/mL, or about 1 pg/mL to 400 pg/mL, or about 1 pg/mL to 350 pg/mL, or about 1 pg/mL to 300 pg/mL, or about 1 pg/mL to 250 pg/mL, or about 1 pg/mL to 200 pg/mL, or about 1 pg/mL to 150 pg/mL, or about 1 pg/mL to 100 pg/mL, or about 1 pg/mL to 90 pg/mL, or about 1 pg/mL to 80 pg/mL, or about 1 pg/mL to 70 pg/mL, or about 1 pg/mL to 60 pg/mL, or about 1 pg/mL to 50 pg/mL, or about 1 pg/mL to 40 pg/mL, or about 1 pg/mL to 30 pg/mL, or about 1 pg/mL to 20 pg/mL, or about 1 pg/mL to 10 pg/mL, or about 5 pg/mL to 1000 pg/mL, or about 5 pg/mL to 800 pg/mL, or about 5 pg/mL to 600 pg/mL, or about 5 pg/mL to 500 pg/mL, or about 10 pg/mL to 1000 pg/mL, or about 10 pg/mL to 500 pg/mL, or about 10 pg/mL to 100 pg/mL, or about 20 pg/mL to 1000 pg/mL, or about 20 pg/mL to 500 pg/mL, or about 20 pg/mL to 200 pg/mL, or about 50 pg/mL to 1000 pg/mL, or about 50 pg/mL to 500 pg/mL, or about 50 pg/mL to 300 pg/mL, or about 100 pg/mL to 1000 pg/mL, or about 100 pg/mL to 500 pg/mL, or greater than 1 pg/mL, or greater than 5 pg/mL, or greater than 10 pg/mL, or greater than 20 pg/mL, or greater than 30 pg/mL, or greater than 40 pg/mL, or greater than 50 pg/mL, or greater than 70 pg/mL, or greater than 100 pg/mL, or greater than 200 pg/mL, or greater than 300 pg/mL. In some preferred embodiments, the aforementioned Cₘᵢₙ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a mean buprenorphine and norbuprenorphine Cₘᵢₙ of less than about 1000 pg/mL. In other preferred embodiments, the dosage form provides a mean buprenorphine and norbuprenorphine Cₘᵢₙ of less than about 900 pg/mL, or less than about 800 pg/mL, or less than about 700 pg/mL, or less than about 600 pg/mL, or less than about 500 pg/mL, or less than about 400 pg/mL, or less than about 300 pg/mL, or less than about 200 pg/mL, or less than about 100 pg/mL, or less than about 90 pg/mL, or less than about 80 pg/mL, or less than about 70 pg/mL, or less than about 60 pg/mL, or less than about 50 pg/mL, or less than about 40 pg/mL, or less than about 30 pg/mL, or less than about 20 pg/mL, or less than about 10 pg/mL, or less than about 5 pg/mL, or less than about 1 pg/mL. In some preferred embodiments, the aforementioned Cₘᵢₙ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a mean buprenorphine and norbuprenorphine Cₘᵢₙ of greater than about 600 pg/mL, or greater than about 500 pg/mL, or greater than about 400 pg/mL, or greater than about 300 pg/mL, or greater than about 200 pg/mL, or greater than about 100 pg/mL, or greater than about 90 pg/mL, or greater than about 80 pg/mL, or greater than about 70 pg/mL, or greater than about 60 pg/mL, or greater than about 50 pg/mL, or greater than about 40 pg/mL, or greater than about 30 pg/mL, or greater than about 20 pg/mL, or greater than about 10 pg/mL, or greater than about 5 pg/mL, or greater than about 1 pg/mL.

In some preferred embodiments, the dosage form provides an oral pharmaceutical composition comprising a therapeutically effective amount of buprenorphine and, optionally, controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a greater than about 60%, 65%, 70%, 75%, 80%, 85%, 90%, 95%, or 100% of the total absorbed dose into systemic circulation (as measured by bioavailability) during the first half of the intended dosing frequency.

In some preferred embodiments, the dosage form provides an oral pharmaceutical composition comprising a therapeutically effective amount of buprenorphine and, optionally, controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a greater than about 60%, 65%, 70%, 75%, 80%, 85%, 90%, 95%, or 100% of the total absorbed dose into systemic circulation (as measured by bioavailability) during the second half of the intended dosing frequency.

In some preferred embodiments, the dosage form provides an oral pharmaceutical composition comprising a therapeutically effective amount of buprenorphine and, optionally, controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a greater than about 50%, 55%, 60%, 65%, 70%, 75%, 80%, 85%, 90%, 95%, or 100% of the total absorbed dose into systemic circulation (as measured by bioavailability) during the first one-third of the intended dosing frequency.

In some preferred embodiments, the dosage form provides an oral pharmaceutical composition comprising a therapeutically effective amount of buprenorphine and, optionally, controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a greater than about 50%, 55%, 60%, 65%, 70%, 75%, 80%, 85%, 90%, 95%, or 100% of the total absorbed dose into systemic circulation (as measured by bioavailability) during the last one-third of the intended dosing frequency.

In some preferred embodiments, the dosage form provides an oral pharmaceutical composition comprising a therapeutically effective amount of buprenorphine and, optionally, controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a greater than about 50%, 55%, 60%, 65%, 70%, 75%, 80%, 85%, 90%, 95%, or 100% of the total absorbed dose into systemic circulation (as measured by bioavailability) during the first one-quarter of the intended dosing frequency.

In some preferred embodiments, the dosage form provides an oral pharmaceutical composition comprising a therapeutically effective amount of buprenorphine and, optionally, controlled release material to render said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form after administration to a human patient providing a greater than about 50%, 55%, 60%, 65%, 70%, 75%, 80%, 85%, 90%, 95%, or 100% of the total absorbed dose into systemic circulation (as measured by bioavailability) during the last one-quarter of the intended dosing frequency.

In some preferred embodiments, in order to attain the specifications and claims of the invention, it is necessary or critical to incorporate a controlled release material in the dosage form.

In some preferred embodiments, the aforementioned embodiments which provide a greater amount of the total absorbed dose into systemic circulation (as measured by bioavailability) during the first half, first one-third or first one quarter of the intended dosing frequency result in reduced frequency or duration of buprenorphine related side effects.

In some preferred embodiments, the aforementioned embodiments which provide a greater amount of the total absorbed dose into systemic circulation (as measured by bioavailability) during the second half, last one-third or last one quarter of the intended dosing frequency result in reduced frequency or duration of buprenorphine related side effects.

In some preferred embodiments, the dosage form comprises a therapeutically effective amount of oral buprenorphine or a pharmaceutically acceptable salt of buprenorphine, or a mixture thereof, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the in vivo specifications, including pharmacokinetic specifications achieved with oral pharmaceutical compositions which are devoid of a controlled release material to render said dosage form extended release.

In some preferred embodiments, the dosage form comprises a therapeutically effective amount of oral buprenorphine or a pharmaceutically acceptable salt of buprenorphine, or a mixture thereof, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the in vitro specifications, including dissolution rate specifications achieved with oral pharmaceutical compositions which are devoid of a controlled release material to render said dosage form extended release.

Pharmacokinetic parameters in the specifications (e.g., AUC, Cmax) referring to “buprenorphine and norbuprenorphine” mean buprenorphine alone and norbuprenorphine alone and not a summation of the two.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN), once-a-day (QD, Q24H or Q24H PRN), once every two days (Q2D, Q48H or Q48H PRN), once every three days (Q3D, Q72H or Q72H PRN), once every four days, once every five days, and up to once every week (QWeekly, Q168H or Q168H PRN) administration to a human patient.

In some preferred embodiments, the specifications of the invention, including in vitro specifications (e.g., dissolution rates) and in vivo specifications (e.g., pharmacodynamic measures) are applicable to dosage forms having dosing frequency (scheduled or PRN) that exceeds 24 hours for up to once-a-week (e.g., the dosage form is suitable once every two days (Q2D, Q48H or Q48H PRN), once every three days (Q3D, Q72H or Q72H PRN), once every four days, once every five days, and up to once every week (QWeekly, Q168H or Q168H PRN) administration to a human patient).

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN), once-a-day (QD, Q24H or Q24H PRN), once every two days (Q2D, Q48H or Q48H PRN), once every three days (Q3D, Q72H or Q72H PRN), once every four days, once every five days, and up to once every week (QWeekly, Q168H or Q168H PRN) administration to a human patient, wherein the in-vitro release rate is substantially independent of pH in that a difference, at any given time, between an amount of buprenorphine released at one pH and an amount released at any other pH, when measured in-vitro using the USP Basket or Paddle Method of USP Drug Release test of U.S. Pharmacopeia (2003) at 100 rpm in 900 ml aqueous buffer, is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the buprenorphine and norbuprenorphine Cₘₐₓ is substantially independent of food intake in that a difference, at any given time, between the Cₘₐₓ of buprenorphine administered in fasted state and the Cₘₐₓ of buprenorphine and norbuprenorphine administered in fed state (using a standardized meal) is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the buprenorphine and norbuprenorphine Cₘₐₓ is substantially independent of food intake in that a difference, at any given time, between the Cₘₐₓ of buprenorphine and norbuprenorphine administered in fasted state and the Cₘₐₓ of buprenorphine administered after a standardized high fat meal is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the buprenorphine and norbuprenorphine Cₘₐₓ is substantially independent of alcohol intake in that a difference, at any given time, between the Cₘₐₓ of buprenorphine and norbuprenorphine administered with about 30 to about 24 mL of a 40% ethanol solution and the Cₘₐₓ of buprenorphine administered without concurrent alcohol (i.e., in an alcohol free state) is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the AUC₀₋₁₂, AUC₀₋₂₄ and AUC_{0-inf} after single-dose administration are substantially independent of food intake in that a difference, at any given time, between the said AUC of buprenorphine and norbuprenorphine when the dosage form administered in fasted state and the said AUC of buprenorphine and norbuprenorphine when the dosage form is administered in fed state (using a standardized meal) is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the buprenorphine and norbuprenorphine AUC₀₋₁₂, AUC₀₋₂₄ and AUC_{0-inf} is substantially independent of food intake in that a difference, at any given time, between the said AUC when administered in fasted state and the AUC when administered after a standardized high fat meal is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, wherein the buprenorphine and norbuprenorphine AUC₀₋₁, AUC₀₋₂, and AUC₀₋₄ is substantially independent of alcohol intake in that a difference, at any given time, between the said AUC when administered with about 30 to about 24 mL of a 40% ethanol solution and the said AUC when administered without concurrent alcohol (i.e., in an alcohol free state) is no greater than about 30%. In other preferred embodiments, said difference is no greater than about 25%, or 20%, or 18%, or 15%, or 12%, or 10%, or 8%, or 5%.

In some preferred embodiments, the dosage form provides an oral pharmaceutical composition comprising a therapeutically effective amount of buprenorphine and, optionally, controlled release material to render said dosage form suitable for three times-a-day (Q8H or Q8H PRN) administration to a human patient, said dosage form after administration to a human patient providing a Cₘₐₓ of buprenorphine and norbuprenorphine at 0.75 to about 7 hours; and said dosage form providing a therapeutic effect for at least about 8 hours. In other preferred embodiments, the dosage form provides a Cₘₐₓ of buprenorphine and norbuprenorphine at about 0.75 to about 6.5 hours or about 0.75 to about 6 hours, or about 0.75 to about 5 hours, or about 0.75 to about 4 hours, or about 0.75 to about 3.5 hours, or about 0.75 to about 3 hours, or 0.75 to about 2.5 hours, or about 0.75 to about 2 hours, or about 0.75 to about 1.5 hours, or about 1 to about 7 hours, or about 1.5 to about 7 hours, or about 2 to about 7 hours, or about 2.5 to about 7 hours, or 3 to about 7 hours, or about 3.5 to about 7 hours, or about 4 to about 7 hours, or about 4.5 to about 7 hours, or about 5 to about 6 hours, or about 5.5 to about 7 hours, or about 2 to about 6, or about 2.5 to about 5.5 hours, or about 3 to about 5 hours, or about 3 to about 6. In some preferred embodiments, the aforementioned Cₘₐₓ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and, optionally, controlled release material to render said dosage form suitable for three times-a-day (Q8H or Q8H PRN) administration to a human patient, said dosage form after administration to a human patient providing a Cₘᵢₙ of buprenorphine and norbuprenorphine at about 6 to about 10 hours; and said dosage form providing a therapeutic effect for at least about 8 hours. In other preferred embodiments, the dosage form provides a Cₘᵢₙ of buprenorphine and norbuprenorphine at about 6 to about 9 hours, or about 6 to about 8.5 hours, or about 6 to about 8 hours, or about 6 to about 7.5 hours, or about 6 to about 7 hours, or about 6.5 to about 10 hours, or about 7 to about 10 hours, or about 8 to about 10 hours, or about 8 hours. In some preferred embodiments, the aforementioned Cₘᵢₙ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and, optionally, controlled release material to render said dosage form suitable for three times-a-day (Q8H or Q8H PRN) administration to a human patient, said dosage form after administration to a human patient providing a mean buprenorphine and norbuprenorphine AUC_{0-inf} after first administration or AUC₀₋₈ at steady state of about 125 pg·hr/mL to about 40000 pg·hr/mL; and said dosage form providing a therapeutic effect for at least about 8 hours. In other preferred embodiments, the dosage form provides a mean buprenorphine and norbuprenorphine AUC_{0-inf} after first administration or AUC₀₋₈ at steady state of about 125 pg·hr/mL to about 35000 pg·hr/mL, or about 125 pg·hr/mL to about 30000 pg·hr/mL, or about 125 pg·hr/mL to about 28000 pg·hr/mL, or about 125 pg·hr/mL to about 25000 pg·hr/mL, or about 125 pg·hr/mL to about 22000 pg·hr/mL, or about 125 pg·hr/mL to about 20000 pg·hr/mL, or about 125 pg·hr/mL to about 18000 pg·hr/mL, or about 125 pg·hr/mL to about 15000 pg·hr/mL, or about 125 pg·hr/mL to about 12500 pg·hr/mL, or about 125 pg·hr/mL to about 10000 pg·hr/mL, or about 125 pg·hr/mL to about 8000 pg·hr/mL, or about 125 pg·hr/mL to about 7500 pg·hr/mL, or about 125 pg·hr/mL to about 7000 pg·hr/mL, or about 125 pg·hr/mL to about 6000 pg·hr/mL, or about 125 pg·hr/mL to about 5500 pg·hr/mL, or about 125 pg·hr/mL to about 5000 pg·hr/mL, or about 125 pg·hr/mL to about 4000 pg·hr/mL, or about 125 pg·hr/mL to about 3500 pg·hr/mL, or about 125 pg·hr/mL to about 3000 pg·hr/mL, or about 125 pg·hr/mL to about 25000 pg·hr/mL, or about 125 pg·hr/mL to about 2000 pg·hr/mL, or about 125 pg·hr/mL to about 1800 pg·hr/mL, or about 125 pg·hr/mL to about 1500 pg·hr/mL, or about 125 pg·hr/mL to about 1000 pg·hr/mL, or about 125 pg·hr/mL to about 800 pg·hr/mL, or about 125 pg·hr/mL to about 700 pg·hr/mL, or about 125 pg·hr/mL to about 6000 pg·hr/mL, or about 125 pg·hr/mL to about 500 pg·hr/mL, or about 125 pg·hr/mL to about 400 pg·hr/mL, or about 175 pg·hr/mL to about 40000 pg·hr/mL, or about 200 pg·hr/mL to about 40000 pg·hr/mL, or about 300 pg·hr/mL to about 40000 pg·hr/mL, or about 400 pg·hr/mL to about 40000 pg·hr/mL, or about 500 pg·hr/mL to about 40000 pg·hr/mL, or about 600 pg·hr/mL to about 40000 pg·hr/mL, or about 700 pg·hr/mL to about 40000 pg·hr/mL, or about 800 pg·hr/mL to about 40000 pg·hr/mL, or about 1000 pg·hr/mL to about 40000 pg·hr/mL, or about 1200 pg·hr/mL to about 40000 pg·hr/mL, or about 1500 pg·hr/mL to about 40000 pg·hr/mL, or about 2000 pg·hr/mL to about 40000 pg·hr/mL, or about 3000 pg·hr/mL to about 4000 pg·hr/mL, or about 4000 pg·hr/mL to about 40000 pg·hr/mL, or about 5000 pg·hr/mL to about 40000 pg·hr/mL, or about 6000 pg·hr/mL to about 40000 pg·hr/mL, or about 8000 pg·hr/mL to about 40000 pg·hr/mL, or about 10000 pg·hr/mL to about 40000 pg·hr/mL, or about 300 pg·hr/mL to about 30000 pg·hr/mL, or about 300 pg·hr/mL to about 20000 pg·hr/mL, or about 300 pg·hr/mL to about 15000 pg·hr/mL, or about 300 pg·hr/mL to about 10000 pg·hr/mL, or about 300 pg·hr/mL to about 8000 pg·hr/mL, or about 300 pg·hr/mL to about 6000 pg·hr/mL, or about 300 pg·hr/mL to about 5000 pg·hr/mL, or about 300 pg·hr/mL to about 4000 pg·hr/mL, or about 300 pg·hr/mL to about 3000 pg·hr/mL, or about 300 pg·hr/mL to about 2500 pg·hr/mL, or about 300 pg·hr/mL to about 2000 pg·hr/mL, or about 300 pg·hr/mL to about 1500 pg·hr/mL, or about 300 pg·hr/mL to about 1000 pg·hr/mL, or about 600 pg·hr/mL to about 30000 pg·hr/mL, or about 600 pg·hr/mL to about 20000 pg·hr/mL, or about 600 pg·hr/mL to about 15000 pg·hr/mL, or about 600 pg·hr/mL to about 10000 pg·hr/mL, or about 600 pg·hr/mL to about 8000 pg·hr/mL, or about 600 pg·hr/mL to about 6000 pg·hr/mL, or about 600 pg·hr/mL to about 5000 pg·hr/mL, or about 600 pg·hr/mL to about 4000 pg·hr/mL, or about 600 pg·hr/mL to about 3000 pg·hr/mL, or about 600 pg·hr/mL to about 2500 pg·hr/mL, or about 600 pg·hr/mL to about 2000 pg·hr/mL, or about 600 pg·hr/mL to about 1500 pg·hr/mL, or about 600 pg·hr/mL to about 1000 pg·hr/mL, or about 1000 pg·hr/mL to about 30000 pg·hr/mL, or about 1000 pg·hr/mL to about 20000 pg·hr/mL, or about 1000 pg·hr/mL to about 15000 pg·hr/mL, or about 1000 pg·hr/mL to about 10000 pg·hr/mL, or about 1000 pg·hr/mL to about 8000 pg·hr/mL, or about 1000 pg·hr/mL to about 10000 pg·hr/mL, or about 1000 pg·hr/mL to about 5000 pg·hr/mL, or about 1000 pg·hr/mL to about 4000 pg·hr/mL, or about 1000 pg·hr/mL to about 3000 pg·hr/mL, or about 1000 pg·hr/mL to about 2500 pg·hr/mL, or about 1000 pg·hr/mL to about 2000 pg·hr/mL, or about 1000 pg·hr/mL to about 1500 pg·hr/mL, or about 2000 pg·hr/mL to about 30000 pg·hr/mL, or about 2000 pg·hr/mL to about 20000 pg·hr/mL, or about 2000 pg·hr/mL to about 15000 pg·hr/mL, or about 2000 pg·hr/mL to about 20000 pg·hr/mL, or about 2000 pg·hr/mL to about 8000 pg·hr/mL, or about 2000 pg·hr/mL to about 20000 pg·hr/mL, or about 2000 pg·hr/mL to about 5000 pg·hr/mL, or about 2000 pg·hr/mL to about 4000 pg·hr/mL, or about 2000 pg·hr/mL to about 3000 pg·hr/mL, or about 2000 pg·hr/mL to about 2500 pg·hr/mL, or about 2000 pg·hr/mL to about 2000 pg·hr/mL, or about 2000 pg·hr/mL to about 1500 pg·hr/mL, or about 4000 pg·hr/mL to about 30000 pg·hr/mL, or about 4000 pg·hr/mL to about 20000 pg·hr/mL, or about 6000 pg·hr/mL to about 15000 pg·hr/mL, or about 6000 pg·hr/mL to about 15000 pg·hr/mL, or about 100 pg·hr/mL to about 4000 pg·hr/mL, or about 100 pg·hr/mL to about 3000 pg·hr/mL. In some preferred embodiments, the aforementioned AUC being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for three times-a-day (Q8H or Q8H PRN) administration to a human patient, said dosage form after administration to a human patient providing a C₈/Cₘₐₓ ratio of buprenorphine and norbuprenorphine 0.1 to about 1.25; and said dosage form providing a therapeutic effect for at least about 8 hours. In other preferred embodiments, the dosage form provides a C₈/Cₘₐₓ ratio of buprenorphine and norbuprenorphine of about 0.1 to about 1, or about 0.1 to about 0.8, or about 0.1 to about 0.75, or about 0.1 to about 0.6, or 0.1 to about 0.5, or about 0.1 to about 0.4, or about 0.1 to about 0.35, or about 0.25 to about 0.95, or about 0.4 to about 0.95, or about 0.5 to about 0.95, or about 0.65 to about 0.95, or about 0.75 to about 0.95, or about 0.3 to about 0.8, or about 0.4 to about 0.75, or about 0.5 to about 0.75. In some preferred embodiments, the aforementioned C₈/Cₘₐₓ ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for three times-a-day (Q8H or Q8H PRN) administration to a human patient, said dosage form after administration to a human patient, providing a W₅₀ of buprenorphine and norbuprenorphine of 1 to about 7 hours; and said dosage form providing a therapeutic effect for at least about 8 hours. In other preferred embodiments, the dosage form provides a W₅₀ of buprenorphine and norbuprenorphine of about 1 to about 6 hours, or about 1 to about 5 hours, or about 1 to about 5.5 hours, or about 1 to about 5 hours, or 1 to about 4.5 hours, or about 1 to about 4 hours, or about 1 to about 3.5 hours, or about 1 to about 3 hours, or about 1 to about 2.5 hours, or about 1 to about 2 hours, or about 1.5 to about 7 hours, or about 2 to about 6 hours, or 2 to about 5.5 hours, or about 2 to about 5 hours, or about 2 to about 4.5 hours, or about 2 to about 4 hours, or about 2 to about 3.5 hours, or about 2.5 to about 6.5 hours, or about 2.5 to about 6 hours, or about 2.5 to about 5 hours, or about 2.5 to about 4.5 hours, or 3 to about 6.5 hours, or about 3 to about 6 hours, or about 3 to about 5 hours, or about 3 to about 6.5 hours. In some preferred embodiments, the aforementioned W₅₀ ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for three times-a-day (Q8H or Q8H PRN) administration to a human patient, said dosage form after administration to a human patient, providing a HVD of buprenorphine and norbuprenorphine of 1.5 to about 7 hours; and said dosage form providing a therapeutic effect for at least about 8 hours. In other preferred embodiments, the dosage form provides a HVD of buprenorphine and norbuprenorphine of about 1.5 to about 6 hours, or about 1.5 to about 5 hours, or about 1.5 to about 5.5 hours, or about 1.5 to about 5 hours, or 1.5 to about 4.5 hours, or about 1.5 to about 4 hours, or about 1.5 to about 3.5 hours, or about 1.5 to about 3 hours, or about 1.5 to about 2.5 hours, or about 1.5 to about 2 hours, or about 1.5 to about 7 hours, or about 2 to about 6 hours, or 2 to about 5.5 hours, or about 2 to about 5 hours, or about 2 to about 4.5 hours, or about 2 to about 4 hours, or about 2 to about 3.5 hours, or about 2.5 to about 6.5 hours, or about 2.5 to about 6 hours, or about 2.5 to about 5 hours, or about 2.5 to about 4.5 hours, or 3 to about 6.5 hours, or about 3 to about 6 hours, or about 3 to about 5 hours, or about 3 to about 6.5 hours. In some preferred embodiments, the aforementioned HVD ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine, and, optionally, controlled release material to render said dosage form suitable for three times-a-day (Q8H or Q8H PRN) administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 47.5% at 1 hour, from about 10% to about 65% at 2 hours, from about 10% to about 90% at 4 hours, from about 15% to about 95% at 6 hours, and greater than about 65% at 8 hours. In other preferred embodiments, the dosage form provides said an in-vitro release rate of from 0% to about 40% at 1 hour, from about 5% to about 60% at 2 hours, from about 10% to about 70% at 4 hours, from about 15% to about 90% at 6 hours, and greater than about 50% at 8 hours.

In some embodiments, some of the dissolution rate specifications of buprenorphine referred to herein are obtained following pretreatment of the dosage form using the USP Basket or Paddle Method at 100 rpm in 900 mL distilled water at a pH of ≦4.5, ≦5, or ≦5.5 for two hours at 37° C. before a switch to an aqueous buffer at 37° C. at a pH ≧6, or ≧6.8 or a pH ≧7 (instead of the aqueous buffer at a pH of between 1.6 and 7.2), whereupon the clock is reset to time equal to 0.

In some preferred embodiments, the dosage form provides an oral pharmaceutical composition comprising a therapeutically effective amount of buprenorphine, and, optionally, controlled release material to render said dosage form suitable for three times-a-day (Q8H or Q8H PRN) administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 80% at 6 hours, and greater than about 65% at 8 hours; said in-vitro release rate being substantially independent of pH in that a difference, at any given time, between an amount of buprenorphine released at one pH and an amount released at any other pH, when measured in-vitro using the USP Basket or Paddle Method of USP Drug Release test of U.S. Pharmacopeia (2003) at 100 rpm in 900 ml aqueous buffer, is no greater than 30%. In other preferred embodiments, said pH independent in-vitro release rate is from 0% to about 47.5% at 1 hour, from about 10% to about 65% at 2 hours, from about 10% to about 90% at 4 hours, from about 15% to about 95% at 6 hours, and greater than about 65% at 8 hours or from 0% to about 40% at 1 hour, from about 5% to about 60% at 2 hours, from about 10% to about 70% at 4 hours, from about 15% to about 90% at 6 hours, and greater than about 50% at 8 hours.

In some preferred embodiments, the dosage form provides an oral pharmaceutical composition comprising a therapeutically effective amount of buprenorphine and controlled release material to render said dosage form suitable for twice-a-day administration to a human patient, said dosage form after administration to a human patient providing a Cₘₐₓ of buprenorphine and norbuprenorphine at 2 to about 10 hours; and said dosage form providing a therapeutic effect for at least about 12 hours. In other preferred embodiments, the dosage form provides a Cₘₐₓ of buprenorphine and norbuprenorphine at about 2 to about 8 hour or about 2 to about 6 hours, or about 2 to about 5 hours, or about 2 to about 7 hours, or about 2 to about 4.5 hours, or about 2 to about 4 hours, or 2 to about 3.5 hours, or about 2 to about 3 hours, or about 3 to about 10 hours, or about 3.5 to about 10 hours, or about 4 to about 10 hours, or about 4.5 to about 10 hours, or about 5 to about 10 hours, or 5 to about 10 hours, or about 6 to about 10 hours, or about 3 to about 8 hours, or about 3 to about 7 hours, or about 3 to about 6 hours, or about 4 to about 8 hours, or about 4 to about 6. In some preferred embodiments, the aforementioned Cₘₐₓ being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for twice-a-day administration to a human patient, said dosage form after administration to a human patient providing a Cₘᵢₙ of buprenorphine and norbuprenorphine at about 10 to about 14 hours; and said dosage form providing a therapeutic effect for at least about 12 hours. In other preferred embodiments, the dosage form provides a Cₘᵢₙ of buprenorphine and norbuprenorphine at about 10 to about 13 hours, or about 10 to about 12.5 hours, or about 10 to about 12 hours, or about 10 to about 11.5 hours, or about 10 to about 11 hours, or about 10.5 to about 14 hours, or about 11 to about 14 hours, or about 12 to about 14 hours. In some preferred embodiments, the aforementioned Cₘᵢₙ, being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for twice-a-day administration to a human patient, said dosage form after administration to a human patient providing a mean buprenorphine and norbuprenorphine AUC_{0-inf} after first administration or AUC₀₋₁₂ at steady state of about 125 pg·hr/mL to about 250000 pg·hr/mL; and said dosage form providing a therapeutic effect for at least about 12 hours. In other preferred embodiments, the dosage form provides a mean buprenorphine and norbuprenorphine AUC_{0-inf} after first administration or AUC₀₋₁₂ at steady state of about 125 pg·hr/mL to about 200000 pg·hr/mL, or about 125 pg·hr/mL to about 150000 pg·hr/mL, or about 125 pg·hr/mL to about 100000 pg·hr/mL, or about 125 pg·hr/mL to about 50000 pg·hr/mL, or about 125 pg·hr/mL to about 22000 pg·hr/mL, or about 125 pg·hr/mL to about 20000 pg·hr/mL, or about 125 pg·hr/mL to about 18000 pg·hr/mL, or about 125 pg·hr/mL to about 15000 pg·hr/mL, or about 125 pg·hr/mL to about 12500 pg·hr/mL, or about 125 pg·hr/mL to about 10000 pg·hr/mL, or about 125 pg·hr/mL to about 8000 pg·hr/mL, or about 125 pg·hr/mL to about 7500 pg·hr/mL, or about 125 pg·hr/mL to about 7000 pg·hr/mL, or about 125 pg·hr/mL to about 6000 pg·hr/mL, or about 125 pg·hr/mL to about 5500 pg·hr/mL, or about 125 pg·hr/mL to about 5000 pg·hr/mL, or about 125 pg·hr/mL to about 4000 pg·hr/mL, or about 125 pg·hr/mL to about 3500 pg·hr/mL, or about 125 pg·hr/mL to about 3000 pg·hr/mL, or about 125 pg·hr/mL to about 25000 pg·hr/mL, or about 125 pg·hr/mL to about 2000 pg·hr/mL, or about 125 pg·hr/mL to about 1800 pg·hr/mL, or about 125 pg·hr/mL to about 1500 pg·hr/mL, or about 125 pg·hr/mL to about 1000 pg·hr/mL, or about 125 pg·hr/mL to about 800 pg·hr/mL, or about 125 pg·hr/mL to about 700 pg·hr/mL, or about 125 pg·hr/mL to about 6000 pg·hr/mL, or about 125 pg·hr/mL to about 500 pg·hr/mL, or about 125 pg·hr/mL to about 400 pg·hr/mL, or about 175 pg·hr/mL to about 40000 pg·hr/mL, or about 200 pg·hr/mL to about 40000 pg·hr/mL, or about 300 pg·hr/mL to about 40000 pg·hr/mL, or about 400 pg·hr/mL to about 40000 pg·hr/mL, or about 500 pg·hr/mL to about 40000 pg·hr/mL, or about 600 pg·hr/mL to about 40000 pg·hr/mL, or about 700 pg·hr/mL to about 40000 pg·hr/mL, or about 800 pg·hr/mL to about 40000 pg·hr/mL, or about 1000 pg·hr/mL to about 40000 pg·hr/mL, or about 1200 pg·hr/mL to about 40000 pg·hr/mL, or about 1500 pg·hr/mL to about 40000 pg·hr/mL, or about 2000 pg·hr/mL to about 40000 pg·hr/mL, or about 3000 pg·hr/mL to about 4000 pg·hr/mL, or about 4000 pg·hr/mL to about 40000 pg·hr/mL, or about 5000 pg·hr/mL to about 40000 pg·hr/mL, or about 6000 pg·hr/mL to about 40000 pg·hr/mL, or about 8000 pg·hr/mL to about 40000 pg·hr/mL, or about 10000 pg·hr/mL to about 40000 pg·hr/mL, or about 300 pg·hr/mL to about 30000 pg·hr/mL, or about 300 pg·hr/mL to about 20000 pg·hr/mL, or about 300 pg·hr/mL to about 15000 pg·hr/mL, or about 300 pg·hr/mL to about 10000 pg·hr/mL, or about 300 pg·hr/mL to about 8000 pg·hr/mL, or about 300 pg·hr/mL to about 6000 pg·hr/mL, or about 300 pg·hr/mL to about 5000 pg·hr/mL, or about 300 pg·hr/mL to about 4000 pg·hr/mL, or about 300 pg·hr/mL to about 3000 pg·hr/mL, or about 300 pg·hr/mL to about 2500 pg·hr/mL, or about 300 pg·hr/mL to about 2000 pg·hr/mL, or about 300 pg·hr/mL to about 1500 pg·hr/mL, or about 300 pg·hr/mL to about 1000 pg·hr/mL, or about 600 pg·hr/mL to about 30000 pg·hr/mL, or about 600 pg·hr/mL to about 20000 pg·hr/mL, or about 600 pg·hr/mL to about 15000 pg·hr/mL, or about 600 pg·hr/mL to about 10000 pg·hr/mL, or about 600 pg·hr/mL to about 8000 pg·hr/mL, or about 600 pg·hr/mL to about 6000 pg·hr/mL, or about 600 pg·hr/mL to about 5000 pg·hr/mL, or about 600 pg·hr/mL to about 4000 pg·hr/mL, or about 600 pg·hr/mL to about 3000 pg·hr/mL, or about 600 pg·hr/mL to about 2500 pg·hr/mL, or about 600 pg·hr/mL to about 2000 pg·hr/mL, or about 600 pg·hr/mL to about 1500 pg·hr/mL, or about 600 pg·hr/mL to about 1000 pg·hr/mL, or about 1000 pg·hr/mL to about 30000 pg·hr/mL, or about 1000 pg·hr/mL to about 20000 pg·hr/mL, or about 1000 pg·hr/mL to about 15000 pg·hr/mL, or about 1000 pg·hr/mL to about 10000 pg·hr/mL, or about 1000 pg·hr/mL to about 8000 pg·hr/mL, or about 1000 pg·hr/mL to about 10000 pg·hr/mL, or about 1000 pg·hr/mL to about 5000 pg·hr/mL, or about 1000 pg·hr/mL to about 4000 pg·hr/mL, or about 1000 pg·hr/mL to about 3000 pg·hr/mL, or about 1000 pg·hr/mL to about 2500 pg·hr/mL, or about 1000 pg·hr/mL to about 2000 pg·hr/mL, or about 1000 pg·hr/mL to about 1500 pg·hr/mL, or about 2000 pg·hr/mL to about 30000 pg·hr/mL, or about 2000 pg·hr/mL to about 20000 pg·hr/mL, or about 2000 pg·hr/mL to about 15000 pg·hr/mL, or about 2000 pg·hr/mL to about 20000 pg·hr/mL, or about 2000 pg·hr/mL to about 8000 pg·hr/mL, or about 2000 pg·hr/mL to about 20000 pg·hr/mL, or about 2000 pg·hr/mL to about 5000 pg·hr/mL, or about 2000 pg·hr/mL to about 4000 pg·hr/mL, or about 2000 pg·hr/mL to about 3000 pg·hr/mL, or about 2000 pg·hr/mL to about 2500 pg·hr/mL, or about 2000 pg·hr/mL to about 2000 pg·hr/mL, or about 2000 pg·hr/mL to about 1500 pg·hr/mL, or about 4000 pg·hr/mL to about 30000 pg·hr/mL, or about 4000 pg·hr/mL to about 20000 pg·hr/mL, or about 6000 pg·hr/mL to about 15000 pg·hr/mL, or about 6000 pg·hr/mL to about 15000 pg·hr/mL, or about 100 pg·hr/mL to about 4000 pg·hr/mL, or about 100 pg·hr/mL to about 3000 pg·hr/mL. In some preferred embodiments, the aforementioned AUC being achieved with oral pharmaceutical compositions devoid of a controlled release material to render said dosage form extended release.

In some preferred embodiments, the dosage form provides an oral pharmaceutical composition comprising a therapeutically effective amount of buprenorphine and controlled release material to render said dosage form suitable for twice-a-day administration to a human patient, said dosage form after administration to a human patient providing a C₁₂/Cₘₐₓ ratio of buprenorphine and norbuprenorphine 0.1 to about 1.25; and said dosage form providing a therapeutic effect for at least about 12 hours. In other preferred embodiments, the dosage form provides a C₁₂/Cₘₐₓ ratio of buprenorphine and norbuprenorphine of about 0.1 to about 1, or about 0.1 to about 0.8, or about 0.1 to about 0.75, or about 0.1 to about 0.6, or 0.1 to about 0.5, or about 0.1 to about 0.4, or about 0.1 to about 0.35, or about 0.25 to about 0.95, or about 0.4 to about 0.95, or about 0.5 to about 0.95, or about 0.65 to about 0.95, or about 0.75 to about 0.95, or about 0.3 to about 0.8, or about 0.4 to about 0.75, or about 0.5 to about 0.75. In some preferred embodiments, the aforementioned C₁₂/Cₘₐₓ ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine and, optionally, controlled release material to render said dosage form suitable for twice-a-day administration to a human patient, said dosage form after administration to a human patient, providing a W₅₀ of buprenorphine and norbuprenorphine of 2 to about 11 hours; and said dosage form providing a therapeutic effect for at least about 12 hours. In other preferred embodiments, the dosage form provides a W₅₀ of buprenorphine and norbuprenorphine of about 2 to about 10 hours, or about 2 to about 9 hours, or about 2 to about 9 hours, or about 2 to about 8 hours, or 2 to about 7 hours, or about 2 to about 6 hours, or about 2 to about 5 hours, or about 2 to about 4 hours, or about 3 to about 10 hours, or about 4 to about 10 hours, or about 5 to about 10 hours, or about 6 to about 10 hours, or 7 to about 10 hours, or about 3 to about 8 hours, or about 4 to about 8 hours, or about 4 to about 7 hours, or about 3 to about 6 hours.

In some preferred embodiments, the dosage form provides an oral pharmaceutical composition comprising a therapeutically effective amount of buprenorphine and, optionally, controlled release material to render said dosage form suitable for twice-a-day administration to a human patient, said dosage form after administration to a human patient, providing a HVD of buprenorphine and norbuprenorphine of 1.5 to about 10 hours; and said dosage form providing a therapeutic effect for at least about 12 hours. In other preferred embodiments, the dosage form provides an HVD of buprenorphine and norbuprenorphine of about 1.5 to about 9 hours, or about 1.5 to 8 hours, or about 1.5 to about 7 hours, or about 1.5 to 6 hours, or about 1.5 to about 5 hours, or about 1.5 to about 4 hours, or about 2 to about 10 hours, or about 3 to 10 hours, or about 4 to about 10 hours, or about 5 to 10 hours, or about 6 to about 10 hours, or about 8 to 10 hours, about 3 to about 8 hours, or about 4 to 8 hours, or about 5 to about 7 hours, or about 3 to 6 hours, or about 3 to about 8 hours, or about 5 to about 8 hours.

In some preferred embodiments, the dosage form provides an oral pharmaceutical composition comprising a therapeutically effective amount of buprenorphine, and, optionally, controlled release material to render said dosage form suitable for twice-a-day administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours. In other preferred embodiments, the dosage form provides said an in-vitro release rate of from 0% to about 40% at 1 hour, from about 5% to about 55% at 2 hours, from about 10% to about 60% at 4 hours, from about 15% to about 70% at 6 hours, from about 25% to about 80% at 9 hours, and greater than about 50% at 12 hours

In some preferred embodiments, the dosage form provides an oral pharmaceutical composition comprising a therapeutically effective amount of buprenorphine, and, optionally, controlled release material to render said dosage form suitable for twice-a-day administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours.

In some preferred embodiments, the dosage form provides an oral pharmaceutical composition comprising a therapeutically effective amount of buprenorphine, and, optionally, controlled release material to render said dosage form suitable for twice-a-day administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours; said in-vitro release rate being substantially independent of pH in that a difference, at any given time, between an amount of buprenorphine released at one pH and an amount released at any other pH, when measured in-vitro using the USP Basket or Paddle Method of USP Drug Release test of U.S. Pharmacopeia (2003) at 100 rpm in 900 ml aqueous buffer, is no greater than 30%.

In some preferred embodiments, the dosage form provides an oral pharmaceutical composition comprising a therapeutically effective amount of buprenorphine and, optionally, controlled release material to render said dosage form suitable for once-a-day administration to a human patient, said dosage form after administration to a human patient providing a Cₘₐₓ of buprenorphine and norbuprenorphine at about 3 to about 20 hours; and said dosage form providing a therapeutic effect for at least about 24 hours. In some preferred embodiments, the dosage form provides a Cₘₐₓ at about 3 to about 18 hours, or about 3 to about 15 hours, or about 3 to about 12 hours, or at about 3 to about 10 hours, or at about 3 to about 8 hours, or at about 3 to about 7 hours, or at about 3 to about 7 hours, or about 4 to about 20 hours, or about 5 to about 20 hours, or about 6 to about 20 hours, or at about 8 to about 20 hours, or at about 10 to about 20 hours, or at about 12 to about 20 hours, or at about 14 to about 20 hours, or about 18 to about 20 hours, or about 4 to about 18 hours, or about 4 to about 16 hours, or at about 4 to about 12 hours, or at about 4 to about 8 hours, or at about 4 to about 10 hours, or at about 3 to about 6 hours.

In some preferred embodiments, the dosage form provides an oral pharmaceutical composition comprising a therapeutically effective amount of buprenorphine and, optionally, controlled release material to render said dosage form suitable for once-a-day administration to a human patient, said dosage form after administration to a human patient providing a Cₘᵢₙ of buprenorphine and norbuprenorphine at about 20 to about 28 hours; and said dosage form providing a therapeutic effect for at least about 24 hours. In some preferred embodiments, the dosage form provides a Cₘᵢₙ at about 20 to about 26 hours, or about 20 to about 27 hours, or about 20 to about 25 hours, or about 20 to about 24 hours, or about 20 to about 23 hours, or about 21 to about 28 hours, or about 22 to about 28 hours, or about 23 to about 28 hours, or about 23.5 to about 28 hours, or about 22 to 26 hours.

In some preferred embodiments, the dosage form provides an oral pharmaceutical composition comprising a therapeutically effective amount of buprenorphine; said dosage form providing a therapeutic effect longer than would be expected based on the prevailing plasma concentrations. For example, under normal circumstances, many drugs provide duration of effect that is at least partly correlated with or dependent on the prevailing plasma concentrations of drug. In some preferred embodiments of the invention, the dosage form provides persistent therapeutic effects despite short lived, low or negligible prevailing plasma concentrations.

In some preferred embodiments of the invention, the dosage form provides sustained therapeutic effects of up to about 4, or about 6, or about 8, or about 12, or about 18 or about 20 or about 24 hours despite being administered in immediate release form.

In some preferred embodiments, the dosage form provides an oral pharmaceutical composition comprising a therapeutically effective amount of buprenorphine; said dosage from providing a Cmax of buprenorphine and norbuprenorphine from about 0.25 hours to about 30 hours.

In some preferred embodiments, the dosage form provides an oral pharmaceutical composition comprising a therapeutically effective amount of buprenorphine; said dosage from providing a Cₘᵢₙ of buprenorphine and norbuprenorphine from about 1 hour to about 30 hours.

In some preferred embodiments, the dosage form provides an oral pharmaceutical composition comprising a therapeutically effective amount of buprenorphine and controlled release material to render said dosage form suitable for once-a-day administration to a human patient, said dosage form after administration to a human patient providing a buprenorphine and norbuprenorphine mean AUC_{0-inf} after first administration or AUC₀₋₂₄ at steady state of about 2250 pg·hr/mL to about 500000 pg·hr/mL; and said dosage form providing a therapeutic effect for at least about 24 hours. In other preferred embodiments, the dosage form provides a mean buprenorphine and norbuprenorphine AUC_{0-inf} after first administration or AUC₀₋₂₄ at steady state of about 250 pg·hr/mL to about 350000 pg·hr/mL, or about 250 pg·hr/mL to about 250000 pg·hr/mL, or about 250 pg·hr/mL to about 150000 pg·hr/mL, or about 250 pg·hr/mL to about 100000 pg·hr/mL, or about 250 pg·hr/mL to about 50000 pg·hr/mL, or about 250 pg·hr/mL to about 25000 pg·hr/mL, or about 250 pg·hr/mL to about 20000 pg·hr/mL, or about 250 pg·hr/mL to about 18000 pg·hr/mL, or about 250 pg·hr/mL to about 15000 pg·hr/mL, or about 250 pg·hr/mL to about 14000 pg·hr/mL, or about 250 pg·hr/mL to about 12000 pg·hr/mL, or about 250 pg·hr/mL to about 11000 pg·hr/mL, or about 250 pg·hr/mL to about 10000 pg·hr/mL, or about 250 pg·hr/mL to about 9000 pg·hr/mL, 250 pg·hr/mL to about 8500 pg·hr/mL, or about 250 pg·hr/mL to about 7000 pg·hr/mL, or about 250 pg·hr/mL to about 5000 pg·hr/mL, or about 250 pg·hr/mL to about 4000 pg·hr/mL, or about 250 pg·hr/mL to about 3000 pg·hr/mL, or about 500 pg·hr/mL to about 70000 pg·hr/mL, or about 500 pg·hr/mL to about 60000 pg·hr/mL, or about 500 pg·hr/mL to about 50000 pg·hr/mL, or about 500 pg·hr/mL to about 40000 pg·hr/mL, or about 500 pg·hr/mL to about 30000 pg·hr/mL, or about 500 pg·hr/mL to about 25000 pg·hr/mL, or about 500 pg·hr/mL to about 20000 pg·hr/mL, or about 500 pg·hr/mL to about 18000 pg·hr/mL, or about 500 pg·hr/mL to about 15000 pg·hr/mL, or about 500 pg·hr/mL to about 14000 pg·hr/mL, or about 500 pg·hr/mL to about 12000 pg·hr/mL, or about 500 pg·hr/mL to about 11000 pg·hr/mL, or about 500 pg·hr/mL to about 10000 pg·hr/mL, or about 500 pg·hr/mL to about 9000 pg·hr/mL, 500 pg·hr/mL to about 8500 pg·hr/mL, or about 500 pg·hr/mL to about 7000 pg·hr/mL, or about 500 pg·hr/mL to about 5000 pg·hr/mL, or about 500 pg·hr/mL to about 4000 pg·hr/mL, or about 500 pg·hr/mL to about 3000 pg·hr/mL, or about 1000 pg·hr/mL to about 70000 pg·hr/mL, or about 1000 pg·hr/mL to about 60000 pg·hr/mL, or about 1000 pg·hr/mL to about 50000 pg·hr/mL, or about 1000 pg·hr/mL to about 40000 pg·hr/mL, or about 1000 pg·hr/mL to about 30000 pg·hr/mL, or about 1000 pg·hr/mL to about 25000 pg·hr/mL, or about 1000 pg·hr/mL to about 20000 pg·hr/mL, or about 1000 pg·hr/mL to about 18000 pg·hr/mL, or about 1000 pg·hr/mL to about 15000 pg·hr/mL, or about 1000 pg·hr/mL to about 14000 pg·hr/mL, or about 1000 pg·hr/mL to about 12000 pg·hr/mL, or about 1000 pg·hr/mL to about 11000 pg·hr/mL, or about 1000 pg·hr/mL to about 10000 pg·hr/mL, or about 1000 pg·hr/mL to about 9000 pg·hr/mL, 1000 pg·hr/mL to about 81000 pg·hr/mL, or about 1000 pg·hr/mL to about 7000 pg·hr/mL, or about 1000 pg·hr/mL to about 5000 pg·hr/mL, or about 1000 pg·hr/mL to about 4000 pg·hr/mL, or about 1000 pg·hr/mL to about 3000 pg·hr/mL, or about 2000 pg·hr/mL to about 70000 pg·hr/mL, or about 2000 pg·hr/mL to about 60000 pg·hr/mL, or about 2000 pg·hr/mL to about 50000 pg·hr/mL, or about 2000 pg·hr/mL to about 40000 pg·hr/mL, or about 2000 pg·hr/mL to about 30000 pg·hr/mL, or about 2000 pg·hr/mL to about 25000 pg·hr/mL, or about 2000 pg·hr/mL to about 20000 pg·hr/mL, or about 2000 pg·hr/mL to about 18000 pg·hr/mL, or about 2000 pg·hr/mL to about 15000 pg·hr/mL, or about 2000 pg·hr/mL to about 14000 pg·hr/mL, or about 2000 pg·hr/mL to about 12000 pg·hr/mL, or about 2000 pg·hr/mL to about 12000 pg·hr/mL, or about 2000 pg·hr/mL to about 10000 pg·hr/mL, or about 2000 pg·hr/mL to about 9000 pg·hr/mL, 2000 pg·hr/mL to about 82000 pg·hr/mL, or about 2000 pg·hr/mL to about 7000 pg·hr/mL, or about 2000 pg·hr/mL to about 5000 pg·hr/mL, or about 2000 pg·hr/mL to about 4000 pg·hr/mL, or about 2000 pg·hr/mL to about 3000 pg·hr/mL, or about 1500 pg·hr/mL to about 8000 pg·hr/mL, or about 1500 pg·hr/mL to about 6000 pg·hr/mL, 3000 pg·hr/mL to about 10000 pg·hr/mL, or about 3000 pg·hr/mL to about 9000 pg·hr/mL, or about 1200 pg·hr/mL to about 12000 pg·hr/mL, or about 4000 pg·hr/mL to about 9000 pg·hr/mL, or about 4000 pg·hr/mL to about 12000 pg·hr/mL. In some preferred embodiments, the aforementioned AUC_{0-inf} being achieved with oral pharmaceutical compositions comprising a controlled release material to render said dosage form extended release, or delayed onset, extended release, or delayed onset, rapid release, or delayed onset, pulsatile release.

In some preferred embodiments, the dosage form provides an oral pharmaceutical composition comprising a therapeutically effective amount of buprenorphine and controlled release material to render said dosage form suitable for once-a-day administration to a human patient, said dosage form after administration to a human patient providing a buprenorphine and norbuprenorphine mean AUC_{0-inf} after first administration or AUC₀₋₂₄ at steady state of not more than about 80000 pg·hr/mL; and said dosage form providing a therapeutic effect for at least about 24 hours. In other preferred embodiments, the dosage form provides a mean buprenorphine and norbuprenorphine AUC_{0-inf} after first administration or AUC₀₋₂₄ at steady state of not more than about 70000 pg·hr/mL, or not more than about 60000 pg·hr/mL, or not more than about 50000 pg·hr/mL, or not more than about 40000 pg·hr/mL, or not more than about 30000 pg·hr/mL, or not more than about 25000 pg·hr/mL, or not more than about 20000 pg·hr/mL, or not more than about 18000 pg·hr/mL, or not more than about 15000 pg·hr/mL, or not more than about 14000 pg·hr/mL, or not more than about 12000 pg·hr/mL, or not more than about 11000 pg·hr/mL, or not more than about 10000 pg·hr/mL, or not more than about 9000 pg·hr/mL, or not more than about 8500 pg·hr/mL, or not more than about 7000 pg·hr/mL, or not more than about 5000 pg·hr/mL, or not more than about 4000 pg·hr/mL, or not more than about 3000 pg·hr/mL, or not more than about 2500 pg·hr/mL, or not more than about 2000 pg·hr/mL, or not more than about 1500 pg·hr/mL. In some preferred embodiments, the aforementioned AUC_{0-inf} being achieved with oral pharmaceutical compositions comprising a controlled release material to render said dosage form extended release, or delayed onset, extended release, or delayed onset, rapid release, or delayed onset, pulsatile release.

In some preferred embodiments, the dosage form provides an oral pharmaceutical composition comprising a therapeutically effective amount of buprenorphine and controlled release material to render said dosage form suitable for once-a-day administration to a human patient, said dosage form after administration to a human patient providing a C₁₆/C₂₄ ratio of buprenorphine and norbuprenorphine 0.1 to about 1.25; and said dosage form providing a therapeutic effect for at least about 24 hours. In other preferred embodiments, the dosage form provides a C₁₆/C₂₄ ratio of buprenorphine and norbuprenorphine of about 0.1 to about 1, or about 0.1 to about 0.8, or about 0.1 to about 0.75, or about 0.1 to about 0.6, or 0.1 to about 0.5, or about 0.1 to about 0.4, or about 0.1 to about 0.35, or about 0.25 to about 0.95, or about 0.4 to about 0.95, or about 0.5 to about 0.95, or about 0.65 to about 0.95, or about 0.75 to about 0.95, or about 0.3 to about 0.8, or about 0.4 to about 0.75, or about 0.5 to about 0.75. In some preferred embodiments, the aforementioned C₁₆/C₂₄ ratio being achieved with oral pharmaceutical compositions devoid of a controlled release material to render said dosage form extended release.

In some preferred embodiments, the dosage form provides an oral pharmaceutical composition comprising a therapeutically effective amount of buprenorphine and, optionally, controlled release material to render said dosage form suitable for once-a-day administration to a human patient, said dosage form after administration to a human patient, providing a W₅₀ of buprenorphine and norbuprenorphine of 4 to about 22 hours; and said dosage form providing a therapeutic effect for at least about 24 hours. In other preferred embodiments, the dosage form provides a W₅₀ of buprenorphine and norbuprenorphine of about 4 to about 20 hours, or about 4 to about 19 hours, or about 4 to about 18 hours, or 4 to about 16 hours, or 4 to about 14 hours, or about 4 to about 12 hours, or about 4 to about 10 hours, or about 4 to about 8 hours, or about 6 to about 20 hours, or about 8 to about 20 hours, or about 10 to about 20 hours, or about 12 to about 20 hours, or 14 to about 20 hours, or about 6 to about 16 hours, or about 8 to about 16 hours, or about 8 to about 14 hours, or about 6 to about 12 hours.

In some preferred embodiments, the dosage form provides an oral pharmaceutical composition comprising a therapeutically effective amount of buprenorphine and, optionally, controlled release material to render said dosage form suitable for once-a-day administration to a human patient, said dosage form after administration to a human patient, providing a HVD of buprenorphine and norbuprenorphine of 3 to about 20 hours; and said dosage form providing a therapeutic effect for at least about 24 hours. In other preferred embodiments, the dosage form provides an HVD of buprenorphine and norbuprenorphine of about 3 to about 18 hours, or about 3 to 16 hours, or about 3 to about 14 hours, or about 3 to 12 hours, or about 3 to about 10 hours, or about 3 to about 8 hours, or about 4 to about 20 hours, or about 6 to 20 hours, or about 8 to about 20 hours, or about 10 to 20 hours, or about 12 to about 20 hours, or about 16 to 20 hours, about 6 to about 16 hours, or about 8 to 16 hours, or about 10 to about 14 hours, or about 6 to 12 hours, or about 6 to about 16 hours, or about 10 to about 16 hours.

In some preferred embodiments, the dosage form provides an oral pharmaceutical composition comprising a therapeutically effective amount of buprenorphine, and, optionally, controlled release material to render said dosage form suitable for once-a-day administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours.

In some preferred embodiments, the dosage form provides an oral pharmaceutical composition comprising a therapeutically effective amount of buprenorphine, and, optionally, controlled release material to render said dosage form suitable for once-a-day administration to a human patient; said dosage form providing an in-vitro release rate by weight of buprenorphine, when measured by the USP Basket or Paddle Method at 100 rpm in 900 mL aqueous buffer at a pH of between 1.6 and 7.2 at 37° C. of from 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours; said in-vitro release rate being substantially independent of pH in that a difference, at any given time, between an amount of buprenorphine released at one pH and an amount released at any other pH, when measured in-vitro using the USP Basket or Paddle Method of USP Drug Release test of U.S. Pharmacopeia (2003) at 100 rpm in 900 ml aqueous buffer, is no greater than 30%.

In some preferred embodiments, the oral dosage form of the invention provides an in-vitro release of from about 2% to about 50% by weight of the buprenorphine or a pharmaceutically acceptable salt of buprenorphine from the dosage form at one hour when measured by the USP Basket Method at 100 rpm in 700 ml of Simulated Gastric Fluid (SGF) at 37° C. In other preferred embodiments, under the same dissolution conditions, said dosage form provides an in-vitro release rate by weight of the buprenorphine or a pharmaceutically acceptable salt of buprenorphine from the dosage form at one hour from about 2% to about 45%, or from about 2% to about 60%, or from about 5% to about 40%, or from about 5% to about 60%, or from about 10% to about 70%, or from about 10% to about 80%, or from about 15% to about 90%, or from about 60 to about 100%, or from about 80 to about 100%, or greater than about 1%, or greater than about 5%, or greater than about 15%, or greater than about 40%, or greater than about 60%, or greater than about 80%, or greater than about 90%, or greater than about 95%.

In some preferred embodiments, the oral dosage form of the invention provides at least about 5%, or 10%, or 15%, or 20%, or 30% or 40%, or 50% or 60%, or 70%, or 80%, or 100% lower variability in buprenorphine Cₘₐₓ (as defined by the coefficient of variation or C.V.) than after the sublingual dosage form of buprenorphine, each given according to its intended route and method of administration.

In some preferred embodiments, the oral dosage form of the invention provides at least about 5%, or 10%, or 15%, or 20%, or 30% or 40%, or 50% or 60%, or 70%, or 80%, or 100% lower variability in buprenorphine Tₘₐₓ (as defined by the coefficient of variation or C.V.) than after the sublingual dosage form of buprenorphine, each given according to its intended route and method of administration.

In some preferred embodiments, the oral dosage form of the invention provides at least about 5%, or 10%, or 15%, or 20%, or 30% or 40%, or 50% or 60%, or 70%, or 80%, or 100% lower variability in the buprenorphine AUC₀₋₂₄, or AUC₀₋₃₆, or AUC₀₋₄₈, or AUC₀₋₇₂ (as defined by the coefficient of variation or C.V.) than after the sublingual dosage form of buprenorphine, each given according to its intended route and method of administration.

In some preferred embodiments, the oral dosage form of the invention provides a ratio of mean AUC₀₋₄₈ of norbuprenorphine to buprenorphine after the first dose which is at least about 5%, or 10%, or 15%, or 20%, or 30% or 40%, or 50% or 60%, or 70%, or 80%, or 100%, or 120%, or 140%, or 150%, or 170%, or 200%, or 230%, or 250%, or 270%, or 300% greater than said ratio after the same amount of sublingual dosage form of buprenorphine, each given according to its intended route and method of administration.

In some preferred embodiments, the oral dosage form of the invention provides a time to 75% mean Cₘₐₓ of buprenorphine after the first dose which is at least about 5%, or 10%, or 15%, or 20%, or 30% or 40%, or 50% or 60%, or 70%, or 80%, or 100%, or 120%, or 140%, or 150%, or 170%, or 200%, or 230%, or 250%, or 270%, or 300% or 400%, or 500%, or 600%, or 700%, or 1000% longer than said time to mean Cₘₐₓ after the same amount of sublingual dosage form of buprenorphine, each given according to its intended route and method of administration.

In some preferred embodiments, the oral dosage form of the invention provides a mean Tₘₐₓ of buprenorphine after the first dose which is at least about 5%, or 10%, or 15%, or 20%, or 30% or 40%, or 50% or 60%, or 70%, or 80%, or 100%, or 120%, or 140%, or 150%, or 170%, or 200%, or 230%, or 250%, or 270%, or 300% or 400%, or 500%, or 600%, or 700%, or 1000% longer than said Tₘₐₓ after the same amount of sublingual dosage form of buprenorphine, each given according to its intended route and method of administration.

In some preferred embodiments, the oral dosage form of the invention provides a mean Cₘₐₓ of buprenorphine which is at least about 5%, or 10%, or 15%, or 20%, or 30% or 40%, or 50% or 60%, or 70%, or 80%, or 100%, or 120%, or 140%, or 150%, or 170%, or 200%, or 230%, or 250%, or 270%, or 300% less than said Cₘₐₓ after the same amount of sublingual dosage form of buprenorphine, each given according to its intended route and method of administration.

In some preferred embodiments, the oral dosage form of the invention provides a mean Cₘₐₓ ratio of norbuprenorphine to buprenorphine which is at least about 5%, or 10%, or 15%, or 20%, or 30% or 40%, or 50% or 60%, or 70%, or 80%, or 100%, or 120%, or 140%, or 150%, or 170%, or 200%, or 230%, or 250%, or 270%, or 300% greater than said Cₘₐₓ ratio after the same amount of sublingual dosage form of buprenorphine, each given according to its intended route and method of administration.

In some preferred embodiments, the oral dosage form of the invention provides a time to 75% mean Cₘₐₓ of buprenorphine which is about 100% to about 2000% of the time to 75% mean Cₘₐₓ after the same amount of a commercially available immediate release sublingual formulation of buprenorphine listed in FDA's Orange Book or an immediate release dosage form (e.g., solution, suspension, tablet or capsule) given orally.

In some preferred embodiments, a controlled release dosage form of the invention provides a time to mean Cₘₐₓ of buprenorphine which is at least about 1.25, 1.5, 2, 3, 4, 5, 6, 7, 8, 9, 10 or 12 fold greater than the time to mean Cₘₐₓ after the same amount of an oral immediate release buprenorphine solution.

In some preferred embodiments, the oral dosage form of the invention provides a time to mean Cₘₐₓ of buprenorphine which is at least about 1.25, 1.5, 2, 3, 4, 5, 6, 7, 8, 9, 10 or 12 fold greater than the time to mean Cₘₐₓ after the same amount of a commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean Cₘₐₓ of buprenorphine which is at least about 5%, 10%, 15%, 20%, 30%, 40%, 50%, 60%, 70%, 80%, 90%, 100%, 120%, 140%, 150%, 180%, 200%, 230%, 260%, or 300% lower than the mean Cₘₐₓ after the same amount of a commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a ratio of mean AUC₀₋₁₄ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the same amount of a commercially available immediate release sublingual formulation of buprenorphine listed in FDA's Orange Book, each given according to its intended route of administration.

In some preferred embodiments, the oral dosage form of the invention provides a ratio of mean AUC₀₋₂₄ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the same amount of a commercially available immediate release sublingual formulation of buprenorphine listed in FDA's Orange Book, each given according to its intended route of administration.

In some preferred embodiments, the oral dosage form of the invention provides a ratio of mean AUC₀₋₃₆ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the same amount of a commercially available immediate release sublingual formulation of buprenorphine listed in FDA's Orange Book, each given according to its intended route of administration.

In some preferred embodiments, the oral dosage form of the invention provides a ratio of mean AUC₀₋₄₈ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the same amount of a commercially available immediate release sublingual formulation of buprenorphine listed in FDA's Orange Book, each given according to its intended route of administration.

In some preferred embodiments, the oral dosage form of the invention provides a ratio of mean AUC₀₋₆₀ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the same amount of a commercially available immediate release sublingual formulation of buprenorphine listed in FDA's Orange Book, each given according to its intended route of administration.

In some preferred embodiments, the oral immediate release and oral extended release dosage forms provide a ratio of mean AUC₀₋₂₄, or AUC₀₋₃₆, or AUC₀₋₄₈, or AUC₀₋₇₂ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after the transdermal buprenorphine, each given according to its intended route and method of administration.

In some preferred embodiments, the oral immediate release and oral extended release dosage forms provide a ratio of mean AUC₀₋₁₂, or AUC₀₋₂₄, or AUC₀₋₄₈ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after intranasal buprenorphine, each given according to its intended route and method of administration.

In some preferred embodiments, the oral immediate release and oral extended release dosage forms provides a ratio of mean AUC₀₋₂₄, or AUC₀₋₃₆, or AUC₀₋₄₈, or AUC₀₋₇₂ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% greater than the said ratio after a sublingual formulation of buprenorphine, each given according to its intended route and method of administration.

In some preferred embodiments, the oral extended release dosage form provides a ratio of mean AUC₀₋₂₄, or AUC₀₋₃₆, or AUC₀₋₄₈, or AUC₀₋₇₂ of norbuprenorphine to buprenorphine after the first dose which is at least about 5%, 10%, 15%, 20%, 30%, 40%, 50%, 60%, 70%, 80%, 90%, 100%, 120%, 140%, 150%, 180%, 200%, 230%, 260%, or 300% less than the said ratio after the same amount of an oral immediate release formulation of buprenorphine.

In some preferred embodiments, the oral extended release dosage form provides a mean extent of absorption (as measured by AUC₀₋₂₄, or AUC₀₋₃₆, or AUC₀₋₄₈, or AUC₀₋₇₂) of buprenorphine after the first dose which is at least about 5%, 10%, 15%, 20%, 30%, or 40% greater than the said ratio after the same amount of an oral immediate release formulation of buprenorphine.

In some preferred embodiments, the ratio of the mean ratio of the extent of absorption (as measured by AUC₀₋₂₄, or AUC₀₋₃₆, or AUC₀₋₄₈, or AUC₀₋₇₂) of norbuprenorphine to buprenorphine for oral extended release buprenorphine after the first dose is at least about 5%, 10%, 15%, 20%, 30%, 40%, 50%, 60%, 70%, 80%, 90%, 100%, 120%, 140%, 150%, 180%, 200%, 230%, 260%, or 300% less than the said ratio after the first dose of the same amount of an oral immediate release formulation of buprenorphine.

In some preferred embodiments, the oral dosage form of the invention provides a mean drowsiness score in buprenorphine and opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than the mean drowsiness score after the same amount of a commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean nausea score which is at least about 5%, 10%, 15%, 20%, 30%, 40%, 50%, 60%, 70%, 80%, 90%, 100%, 120%, 140%, 150%, 180%, 200%, 230%, 260%, or 300% lower than the mean nausea score after the same amount of a commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean driving simulation impairment score in buprenorphine and opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than the mean driving simulation impairment score after the same amount of a commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean number needed to harm (NNH) due to moderate to severe sedation or drowsiness in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean number needed to harm (NNH) due to moderate or severe nausea in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean number needed to harm (NNH) due to dizziness in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean number needed to harm (NNH) due to dry mouth in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean “drug effects” score in opioid abusers or recreational opioid users which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean “drug liking” score in opioid abusers or recreational opioid users which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean “take again” score in opioid abusers or recreational opioid users which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean “coasting” score in opioid abusers or recreational opioid users which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean “critical tracking task” impairment score in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean “stop signal task” impairment score in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean “Tower of London” (TOL) impairment score in opioid naïve subjects which is at least about 5%, 10%, 15%, 20%, 30%, 40%, 50%, 60%, 70%, 80%, 90%, 100%, 120%, 140%, 150%, 180%, 200%, 230%, 260%, or 300% lower than said score after an equal amount or dose of commercially available immediate release sublingual formulation of buprenorphine listed in FDA's Orange Book or an immediate release dosage form (e.g., solution, suspension, tablet or capsule) given orally.

In some preferred embodiments, the oral dosage form of the invention provides a mean ratio of street price at about 1, or 2, or 3, or 4 or 5, or 6 hours post-dose, after administration of an equal amount or dose of commercially available immediate release sublingual formulation of buprenorphine listed in FDA's Orange Book or an immediate release dosage form (e.g., solution, suspension, tablet or capsule) given orally to the dosage form of the invention is ≧1.10, ≧1.15, or ≧1.25, or ≧1.5, or ≧1.75, or ≧2, or ≧2.5, or ≧3, or ≧3.5, or ≧4, or ≧4.5, or ≧5, or ≧5.5, or ≧6, or ≧6.5, or ≧7, or ≧7.5, or ≧8, or ≧8.5, or ≧9, or ≧9.5, or ≧10, where “street price” is based the price recreational drug users or drug addicts would be prepared to pay after consuming said buprenorphine by the intended method of use or by any method of use.

In some preferred embodiments, the oral dosage form of the invention provides a mean ratio of street price at about 1, or 2, or 3, or 4 or 5, or 6 hours post-dose, after administration of an equal amount or dose of commercially available immediate release sublingual formulation of buprenorphine listed in FDA's Orange Book or an immediate release dosage form (e.g., solution, suspension, tablet or capsule) given orally to the dosage form of the invention is ≧1.10, ≧1.15, or ≧1.25, or ≧1.5, or ≧1.75, or ≧2, or ≧2.5, or ≧3, or ≧3.5, or ≧4, or ≧4.5, or ≧5, or ≧5.5, or ≧6, or ≧6.5, or ≧7, or ≧7.5, or ≧8, or ≧8.5, or ≧9, or ≧9.5, or ≧10, where “street price” is based the price recreational drug users or drug addicts would be prepared to pay after consuming said buprenorphine by the intended method of use or by any method of use, and where said buprenorphine use is followed about 0.5 to 1.5 hours later by alcohol (ethanol) administration sufficient to maintain a blood alcohol concentration of 0.04% to 0.08%.

In some preferred embodiments, the oral dosage form of the invention provides a relative mean Cₘₐₓ whose 90% confidence interval is outside the 80.00% to 125.00, under single-dose fasted test conditions in healthy subjects, when compared with immediate release sublingual formulation of buprenorphine listed in FDA's Orange Book, each given to according to its intended route of administration.

In some preferred embodiments, the oral dosage form of the invention provides a relative mean Cₘₐₓ whose 90% confidence interval is outside the 80.00% to 125.00, under single-dose fasted test conditions in healthy subjects, when compared with other available buprenorphine dosage forms.

In some preferred embodiments, the oral dosage form of the invention provides a relative mean Cₘₐₓ under single-dose fasted test conditions in healthy subjects which is statistically significantly different, when compared with immediate release sublingual formulation of buprenorphine listed in FDA's Orange Book, each given to according to its intended route of administration.

In some preferred embodiments, the oral dosage form of the invention provides a relative mean Cₘₐₓ under single-dose fasted test conditions in healthy subjects which is statistically significantly different, when compared with oral ingestion of a conventional solution, suspension, immediate release tablet or capsule.

In some preferred embodiments, the oral dosage form of the invention provides a relative mean Cₘₐₓ whose 90% confidence interval is outside the 80.00% to 125.00, under single-dose fasted test conditions in healthy subjects, when compared with intranasal dosage forms of buprenorphine dosage forms.

In some preferred embodiments, the oral dosage form of the invention provides a relative mean Cₘₐₓ whose 90% confidence interval is outside the 80.00% to 125.00, under single-dose fasted test conditions in healthy subjects, when compared with transdermal dosage forms of buprenorphine dosage forms.

In some preferred embodiments, the oral dosage form of the invention provides a relative mean Cₘₐₓ whose 90% confidence interval is outside the 80.00% to 125.00, under single-dose fasted test conditions in healthy subjects, when compared with buccal dosage forms of buprenorphine dosage forms.

In some preferred embodiments, the oral dosage form of the invention provides a relative mean Cₘₐₓ whose 90% confidence interval is outside the 80.00% to 125.00, under single-dose fasted test conditions in healthy subjects, when compared with immediate release oral buprenorphine.

In some preferred embodiments, the oral dosage form of the invention provides a relative mean Cₘₐₓ whose 90% confidence interval is outside the 80.00% to 125.00, under single-dose fasted test conditions in healthy subjects, when compared with immediate release oral buprenorphine solution.

In some preferred embodiments, the oral dosage form of the invention provides a relative mean AUC₀₋₆, or AUC₀₋₈, or AUC₀₋₁₂, or AUC₀₋₂₄, or AUC_{0-τ}, or AUC_{0-inf}, under single-dose fasted test conditions in healthy subjects which is statistically significantly different, when compared with immediate release sublingual formulation of buprenorphine listed in FDA's Orange Book, each given to according to its intended route of administration.

In some preferred embodiments, the oral dosage form of the invention provides a relative mean AUC₀₋₆, or AUC₀₋₈, or AUC₀₋₁₂, or AUC₀₋₂₄, or AUC_{0-τ}, or AUC_{0-inf}, under single-dose fasted test conditions in healthy subjects which is statistically significantly different, when compared with oral ingestion of a conventional solution, suspension, immediate release tablet or capsule.

In some preferred embodiments, the oral dosage form of the invention provides a relative mean AUC_{0-τ} whose 90% confidence interval is outside the 80.00% to 125.00, under single-dose fasted test conditions in healthy subjects, when compared with immediate release sublingual formulation of buprenorphine listed in FDA's Orange Book.

In some preferred embodiments, the oral dosage form of the invention provides a relative mean AUC_{0-τ} whose 90% confidence interval is outside the 80.00% to 125.00, under single-dose fasted test conditions in healthy subjects, when compared with immediate release sublingual formulation of buprenorphine listed in FDA's Orange Book, each given to according to its intended route of administration.

In some preferred embodiments, the oral dosage form of the invention provides a relative mean AUC_{0-τ} whose 90% confidence interval is outside the 80.00% to 125.00, under single-dose fasted test conditions in healthy subjects, when compared with other available buprenorphine dosage forms.

In some preferred embodiments, the oral dosage form of the invention provides a relative mean AUC_{0-τ} whose 90% confidence interval is outside the 80.00% to 125.00, under single-dose fasted test conditions in healthy subjects, when compared with intranasal dosage forms of buprenorphine dosage forms.

In some preferred embodiments, the oral dosage form of the invention provides a relative mean AUC_{0-τ} whose 90% confidence interval is outside the 80.00% to 125.00, under single-dose fasted test conditions in healthy subjects, when compared with transdermal dosage forms of buprenorphine dosage forms.

In some preferred embodiments, the oral dosage form of the invention provides a relative mean AUC_{0-τ} whose 90% confidence interval is outside the 80.00% to 125.00, under single-dose fasted test conditions in healthy subjects, when compared with buccal dosage forms of buprenorphine dosage forms.

In some preferred embodiments, the oral dosage form of the invention provides a relative mean AUC_{0-τ} whose 90% confidence interval is outside the 80.00% to 125.00, under single-dose fasted test conditions in healthy subjects, when compared with immediate release oral buprenorphine.

In some preferred embodiments, the oral dosage form of the invention provides a relative mean AUC_{0-τ} whose 90% confidence interval is outside the 80.00% to 125.00, under single-dose fasted test conditions in healthy subjects, when compared with immediate release oral buprenorphine solution.

In some preferred embodiments, the oral dosage form of the invention provides a relative mean AUC_{0-τ} whose 90% confidence interval is outside the 80.00% to 125.00, under single-dose fasted test conditions in healthy subjects, when compared with immediate release sublingual formulation of buprenorphine listed in FDA's Orange Book.

In some preferred embodiments, the oral dosage form of the invention provides a relative mean AUC_{0-inf} whose 90% confidence interval is outside the 80.00% to 125.00, under single-dose fasted test conditions in healthy subjects, when compared with immediate release sublingual formulation of buprenorphine listed in FDA's Orange Book, each given to according to its intended route of administration.

In some preferred embodiments, the oral dosage form of the invention provides a relative mean AUC_{0-inf} whose 90% confidence interval is outside the 80.00% to 125.00, under single-dose fasted test conditions in healthy subjects, when compared with other available buprenorphine dosage forms.

In some preferred embodiments, the oral dosage form of the invention provides a relative mean AUC_{0-inf} whose 90% confidence interval is outside the 80.00% to 125.00, under single-dose fasted test conditions in healthy subjects, when compared with intranasal dosage forms of buprenorphine dosage forms.

In some preferred embodiments, the oral dosage form of the invention provides a relative mean AUC_{0-inf} whose 90% confidence interval is outside the 80.00% to 125.00, under single-dose fasted test conditions in healthy subjects, when compared with transdermal dosage forms of buprenorphine dosage forms.

In some preferred embodiments, the oral dosage form of the invention provides a relative mean AUC_{0-inf} whose 90% confidence interval is outside the 80.00% to 125.00, under single-dose fasted test conditions in healthy subjects, when compared with buccal dosage forms of buprenorphine dosage forms.

In some preferred embodiments, the oral dosage form of the invention provides a relative mean AUC_{0-inf} whose 90% confidence interval is outside the 80.00% to 125.00, under single-dose fasted test conditions in healthy subjects, when compared with immediate release oral buprenorphine.

In some preferred embodiments, the oral dosage form of the invention provides a relative mean AUC_{0-inf} whose 90% confidence interval is outside the 80.00% to 125.00, under single-dose fasted test conditions in healthy subjects, when compared with immediate release oral buprenorphine solution.

In some preferred embodiments, the dosage from maintains a plasma buprenorphine and norbuprenorphine concentration within 50% of Cₘₐₓ for about 1 to about 9 hours, or about 2 to about 9 hours, or about 3 to about 9 hours, or about 4 to about 9 hours, or about 5 to about 9 hours, or about 6 to about 9 hours, or about 1 to about 8 hours, or about 2 to about 8 hours, or about 3 to about 8 hours, or about 4 to about 8 hours, or about 5 to about 8 hours, or about 6 to about 8 hours, or about 2 to about 7 hours, or about 2 to about 6 hours, or about 2 to about 5 hours, or about 2 to about 4 hours, or about 1 to about 4 hours, or about 1 to about 3 hours, or about 2 to about 6 hours, or about 3 to about 5 hours.

In some preferred embodiments, the dosage from maintains a plasma buprenorphine and norbuprenorphine concentration within 50% of Cₘₐₓ for about 1 to about 9 hours, or about 2 to about 9 hours, or about 3 to about 9 hours, or about 4 to about 9 hours, or about 5 to about 9 hours, or about 6 to about 9 hours, or about 1 to about 8 hours, or about 2 to about 8 hours, or about 3 to about 8 hours, or about 4 to about 8 hours, or about 5 to about 8 hours, or about 6 to about 8 hours, or about 2 to about 7 hours, or about 2 to about 6 hours, or about 2 to about 5 hours, or about 2 to about 4 hours, or about 1 to about 4 hours, or about 1 to about 3 hours, or about 2 to about 6 hours, or about 3 to about 5 hours during a 12 hour dosing interval.

In some preferred embodiments, the dosage from maintains a plasma buprenorphine and norbuprenorphine concentration within 50% of Cₘₐₓ for about 1 to about 20 hours, or about 1 to about 18 hours or about 1 to about 16 hours, or about 1 to about 14 hours or about 1 to about 10 hours, or about 1 to about 8 hours or about 1 to about 6 hours, or about 1 to about 5 hours or about 2 to about 20 hours, or about 4 to about 20 hours or about 4 to about 18 hours, or about 5 to about 18 hours or about 6 to about 18 hours, or about 7 to about 18 hours or about 8 to about 18 hours, or about 10 to about 18 hours or about 12 to about 18 hours, or about 14 to about 18 hours or about 4 to about 16 hours, or about 4 to about 12 hours or about 4 to about 10 hours, or about 4 to about 8 hours or about 5 to about 15 hours, or about 5 to about 10 hours or about 6 to about 18 hours, or about 6 to about 12 hours or about 6 to about 10 hours, or about 8 to about 18 hours or about 8 to about 16 hours, or about 10 to about 18 hours.

In some preferred embodiments, the dosage from maintains a plasma buprenorphine and norbuprenorphine concentration within 50% of Cₘₐₓ for about 1 to about 20 hours, or about 1 to about 18 hours or about 1 to about 16 hours, or about 1 to about 14 hours or about 1 to about 10 hours, or about 1 to about 8 hours or about 1 to about 6 hours, or about 1 to about 5 hours or about 2 to about 20 hours, or about 4 to about 20 hours or about 4 to about 18 hours, or about 5 to about 18 hours or about 6 to about 18 hours, or about 7 to about 18 hours or about 8 to about 18 hours, or about 10 to about 18 hours or about 12 to about 18 hours, or about 14 to about 18 hours or about 4 to about 16 hours, or about 4 to about 12 hours or about 4 to about 10 hours, or about 4 to about 8 hours or about 5 to about 15 hours, or about 5 to about 10 hours or about 6 to about 18 hours, or about 6 to about 12 hours or about 6 to about 10 hours, or about 8 to about 18 hours or about 8 to about 16 hours, or about 10 to about 18 hours, during a 24 hour dosing interval.

In some preferred embodiments, the oral pharmaceutical composition of buprenorphine or a pharmaceutically dosage from provides a buprenorphine Tₘₐₓ greater than about 0.25 hours, or greater than about 0.5 hours, or greater than about 0.75 hours, or greater than about 1 hour, or greater than about 1.5 hours, or greater than about 2 hours, or greater than about 2.5 hours, or greater than about 3 hours, or greater than about 3.5 hours, or greater than about 4 hours, or greater than about 4.5 hours, or greater than about 5 hours, or greater than about 6 hours, or greater than about 8 hours, or greater than about 10 hours, or greater than about 12 hours, or greater than about 14 hours, or greater than about 16 hours, or greater than about 17 hours, or greater than about 18 hours, or greater than about 20 hours, or greater than about 22 hours, or greater than about 24 hours.

In some preferred embodiments, the oral pharmaceutical composition of buprenorphine or a pharmaceutically dosage from provides a buprenorphine Tₘₐₓ of about 0.25 to about 8 hours, about 0.5 to about 30 hours, or about 0.5 to about 26 hours, or about 0.5 to about 22 hours, or about 0.5 to about 20 hours, or about 0.5 to about 18 hours, or about 0.5 to about 16 hours, or about 0.5 to about 14 hours, or about 0.5 to about 12 hours, or about 0.5 to about 10 hours, or about 0.5 to about 9 hours, or about 0.5 to about 8 hours, or about 0.5 to about 7 hours, or about 0.5 to about 6 hours, or about 0.5 to about 5 hours, or about 0.5 to about 4 hours, or about 0.5 to about 3 hours, or about 0.5 to about 2 hours, or about 0.5 to about 1 hour, or about 1 to about 30 hours, or about 2 to about 30 hours, or about 3 to about 30 hours, or about 4 to about 30 hours, or about 5 to about 30 hours, or about 6 to about 30 hours, or about 1 to about 24 hours, or about 2 to about 24 hours, or about 3 to about 24 hours, or about 4 to about 24 hours, or about 5 to about 24 hours, or about 6 to about 24 hours, or about 8 to about 24 hours, or about 10 to about 24 hours, or about 12 to about 24 hours, or about 14 to about 24 hours, or about 1.5 to about 16 hours, or about 2 to about 16 hours, or about 2.5 to about 16 hours, or about 3 to about 16 hours, or about 3.5 to about 16 hours, or about 4 to about 16 hours, or about 5 to about 16 hours, or about 6 to about 16 hours, or about 7 to about 16 hours, or about 8 to about 16 hours, or about 10 to about 16 hours, or about 12 to about 16 hours, or about 2 to about 10 hours, or about 2.5 to about 10 hours, or about 3 to about 10 hours, or about 3.5 to about 10 hours, or about 4 to about 10 hours, or about 4.5 to about 10 hours, or about 5 to about 10 hours, or about 6 to about 10 hours, or about 7 to about 10 hours, or about 7.5 to about 10 hours.

In some preferred embodiments, the oral pharmaceutical composition of buprenorphine or a pharmaceutically dosage from provides a norbuprenorphine Tₘₐₓ greater than about 0.25 hours, or greater than about 0.5 hours, or greater than about 0.75 hours, or greater than about 1 hour, or greater than about 1.5 hours, or greater than about 2 hours, or greater than about 2.5 hours, or greater than about 3 hours, or greater than about 3.5 hours, or greater than about 4 hours, or greater than about 4.5 hours, or greater than about 5 hours, or greater than about 6 hours, or greater than about 8 hours, or greater than about 10 hours, or greater than about 12 hours, or greater than about 14 hours, or greater than about 16 hours, or greater than about 17 hours, or greater than about 18 hours, or greater than about 20 hours, or greater than about 22 hours, or greater than about 24 hours.

In some preferred embodiments, the oral pharmaceutical composition of buprenorphine or a pharmaceutically dosage from provides a norbuprenorphine Tₘₐₓ of about 0.25 to about 8 hours, about 0.5 to about 30 hours, or about 0.5 to about 26 hours, or about 0.5 to about 22 hours, or about 0.5 to about 20 hours, or about 0.5 to about 18 hours, or about 0.5 to about 16 hours, or about 0.5 to about 14 hours, or about 0.5 to about 12 hours, or about 0.5 to about 10 hours, or about 0.5 to about 9 hours, or about 0.5 to about 8 hours, or about 0.5 to about 7 hours, or about 0.5 to about 6 hours, or about 0.5 to about 5 hours, or about 0.5 to about 4 hours, or about 0.5 to about 3 hours, or about 0.5 to about 2 hours, or about 0.5 to about 1 hour, or about 1 to about 30 hours, or about 2 to about 30 hours, or about 3 to about 30 hours, or about 4 to about 30 hours, or about 5 to about 30 hours, or about 6 to about 30 hours, or about 1 to about 24 hours, or about 2 to about 24 hours, or about 3 to about 24 hours, or about 4 to about 24 hours, or about 5 to about 24 hours, or about 6 to about 24 hours, or about 8 to about 24 hours, or about 10 to about 24 hours, or about 12 to about 24 hours, or about 14 to about 24 hours, or about 1.5 to about 16 hours, or about 2 to about 16 hours, or about 2.5 to about 16 hours, or about 3 to about 16 hours, or about 3.5 to about 16 hours, or about 4 to about 16 hours, or about 5 to about 16 hours, or about 6 to about 16 hours, or about 7 to about 16 hours, or about 8 to about 16 hours, or about 10 to about 16 hours, or about 12 to about 16 hours, or about 2 to about 10 hours, or about 2.5 to about 10 hours, or about 3 to about 10 hours, or about 3.5 to about 10 hours, or about 4 to about 10 hours, or about 4.5 to about 10 hours, or about 5 to about 10 hours, or about 6 to about 10 hours, or about 7 to about 10 hours, or about 7.5 to about 10 hours.

In some preferred embodiments, the oral dosage form of the invention provides a mean in vivo extent of absorption of buprenorphine from 0 to 4 hours which is at least 20% of the mean in vivo extent of absorption from to 0 to 12 hours, wherein the mean in vivo extent of absorption is the area under the plasma or serum buprenorphine concentration time curve from the time of drug administration to the specified time point.

In some preferred embodiments, the oral dosage form of the invention provides a mean in vivo extent of absorption of buprenorphine from 0 to 8 hours which is at least 20% of the mean in vivo extent of absorption from to 0 to 24 hours, wherein the mean in vivo extent of absorption is the area under the plasma or serum buprenorphine concentration time curve from the time of drug administration to the specified time point.

In some preferred embodiments, the oral dosage form of the invention provides a mean in vivo extent of absorption of buprenorphine and norbuprenorphine over the dosing interval (e.g., from 0 to 12 hours or from 0 to 24 hours) which is at least 40% of the mean in vivo extent of absorption from to 0 to infinity, wherein the mean in vivo extent of absorption is the area under the plasma or serum buprenorphine and norbuprenorphine concentration time curves (AUC) from the time of drug administration to the specified time point and where AUC infinity is the sum of AUC from time “0” to time “t” (the last quantifiable time point which has been sampled) plus the extrapolated AUC from the last quantifiable sampling time point to infinity.

In some preferred embodiments, the oral buprenorphine dosage form provides a mean in vivo extent of absorption of buprenorphine or norbuprenorphine from about 0 to about 2 hours, or about 0 to about 3 hours, or about 0 to about 4 hours, or about 0 to about 5 hours, or about 0 to about 6 hours, which is ≦ about 1% of the mean in vivo extent of absorption from to 0 to 12 hours, wherein the mean in vivo extent of absorption is the area under the plasma or serum buprenorphine or norbuprenorphine concentration time curve from the time of drug administration to the specified time point. In other embodiments, said in vivo extent of absorption from about 0 to about 2 hours, or about 0 to about 3 hours, or about 0 to about 4 hours, or about 0 to about 5 hours, or about 0 to about 6 hours is ≦ about 2%, or ≦ about 3%, or ≦ about 4%, or ≦ about 5%, or ≦ about 6%, or ≦ about 7%, or ≦ about 8%, or ≦ about 9%, or ≦ about 10%, or ≦ about 12%, or ≦ about 14%, or ≦ about 15%, or ≦ about 16%, or ≦ about 18%, or ≦ about 20%, or ≦ about 25%, or ≦ about 30%, or ≦ about 35% of the mean in vivo extent of absorption from to 0 to 12 hours.

In some preferred embodiments, the oral buprenorphine dosage form provides a mean in vivo extent of absorption of buprenorphine or norbuprenorphine from about 0 to about 2 hours, or about 0 to about 3 hours, or about 0 to about 4 hours, or about 0 to about 5 hours, or about 0 to about 6 hours, which is ≦ about 1% of the mean in vivo extent of absorption from to 0 to 24 hours, wherein the mean in vivo extent of absorption is the area under the plasma or serum buprenorphine or norbuprenorphine concentration time curve from the time of drug administration to the specified time point. In other embodiments, said in vivo extent of absorption from about 0 to about 2 hours, or about 0 to about 3 hours, or about 0 to about 4 hours, or about 0 to about 5 hours, or about 0 to about 6 hours is ≦ about 2%, or ≦ about 3%, or ≦ about 4%, or ≦ about 5%, or ≦ about 6%, or ≦ about 7%, or ≦ about 8%, or ≦ about 9%, or ≦ about 10%, or ≦ about 12%, or ≦ about 14%, or ≦ about 15%, or ≦ about 16%, or ≦ about 18%, or ≦ about 20%, or ≦ about 25%, or ≦ about 30%, or ≦ about 35% of the mean in vivo extent of absorption from to 0 to 24 hours.

In some preferred embodiments, the oral buprenorphine dosage form provides a mean in vivo extent of absorption of buprenorphine or norbuprenorphine from about 0 to about 7 hours, or about 0 to about 8 hours, or about 0 to about 9 hours, or about 0 to about 10 hours, which is ≦ about 1% of the mean in vivo extent of absorption from to 0 to 24 hours, wherein the mean in vivo extent of absorption is the area under the plasma or serum buprenorphine or norbuprenorphine concentration time curve from the time of drug administration to the specified time point. In other embodiments, said in vivo extent of absorption from about 0 to about 7 hours, or about 0 to about 8 hours, or about 0 to about 9 hours, or about 0 to about 10 hours is ≦ about 2%, or ≦ about 3%, or ≦ about 4%, or ≦ about 5%, or ≦ about 6%, or ≦ about 7%, or ≦ about 8%, or ≦ about 9%, or ≦ about 10%, or ≦ about 12%, or ≦ about 14%, or ≦ about 15%, or ≦ about 16%, or ≦ about 18%, or ≦ about 20%, or ≦ about 25% or ≦ about 30% or ≦ about 35%, or ≦ about 40%, or ≦ about 45%, or ≦ about 50% of the mean in vivo extent of absorption from to 0 to 24 hours.

In some preferred embodiments, the oral buprenorphine dosage form provides a mean in vivo extent of absorption of buprenorphine or norbuprenorphine from about 0 to about 2 hours, or about 0 to about 3 hours, or about 0 to about 4 hours, or about 0 to about 5 hours, or about 0 to about 6 hours, or about 0 to about 7 hours, or about 0 to about 8 hours, or about 0 to about 9 hours, or about 0 to about 10 hours, which is ≦ about 1% of the mean in vivo extent of absorption from to 0 to 36 hours, wherein the mean in vivo extent of absorption is the area under the plasma or serum buprenorphine or norbuprenorphine concentration time curve from the time of drug administration to the specified time point. In other embodiments, said in vivo extent of absorption from about 0 to about 2 hours, or about 0 to about 3 hours, or about 0 to about 4 hours, or about 0 to about 5 hours, or about 0 to about 6 hours, or about 0 to about 7 hours, or about 0 to about 8 hours, or about 0 to about 9 hours, or about 0 to about 10 hours is ≦ about 2%, or ≦ about 3%, or ≦ about 4%, or ≦ about 5%, or ≦ about 6%, or ≦ about 7%, or ≦ about 8%, or ≦ about 9%, or ≦ about 10%, or ≦ about 12%, or ≦ about 14%, or ≦ about 15%, or ≦ about 16%, or ≦ about 18%, or ≦ about 20%, or ≦ about 25% or ≦ about 30% or ≦ about 35%, or ≦ about 35%, or ≦ about 40% or ≦ about 45%, or ≦ about 50%, or ≦ about 60% of the mean in vivo extent of absorption from to 0 to 36 hours.

In some preferred embodiments, the oral dosage form of the invention provides an in-vitro release rate by weight of buprenorphine of less than 5% at one hour when measured by the USP Basket Method at 100 rpm in 700 ml of Simulated Gastric Fluid (SGF) at 37° C. In other embodiments, said release rate from 2% to about 50% at one hour, or from 5% to about 40% at one hour, or from 5% to about 40% at one hour, or from 5% to about 45% at one hour, or from 10% to about 40% at one hour, or from 20% to about 40% at one hour, or from 1% to about 40% at one hour, or from 1% to about 60% at one hour, or from 1% to about 80% at one hour, or from 1% to about 90% at one hour, or from 1% to about 100% at one hour, or greater than about 1% at one hour, or greater than about 5% at one hour, or greater than about 10% at one hour, or greater than about 20% at one hour, or greater than about 30% at one hour, or greater than about 40% at one hour, or greater than about 50% at one hour, or greater than about 60% at one hour, or greater than about 70% at one hour, or greater than about 80% at one hour, or greater than about 90% at one hour.

In some preferred embodiments, the oral dosage form of the invention provides an in-vitro release of between 0% to about 50% by weight of the buprenorphine or a pharmaceutically acceptable salt of buprenorphine from the dosage form at one hour when measured by the USP Basket or Paddle Method at 100 rpm in 700 ml of Simulated Gastric Fluid (SGF) at 37° C.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of more than about 40% at 10 minutes, more than about 60% at 20 minutes and more than about 80% at 30 minutes

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 80% at 0.5 hours, and greater than about 40% at 1 hour.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 90% at 0.5 hours, and greater than about 60% at 1 hour.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 5% to about 90% at 10 minutes, and greater than about 60% at 20 minutes.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 5% to about 100% at 10 minutes, and greater than about 60% at 30 minutes.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 100% at 0.5 hours, and greater than about 70% at 1 hour.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 90% at 1 hour, and greater than about 40% at 2 hours.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 90% at 1 hour, and greater than about 70% at 2 hours.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 50% at 1 hour, and greater than about 30% at 2 hours.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 30% at 1 hour, and greater than about 25% at 2 hours.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 100% at 0.5 hours, and greater than about 60% at 1 hour.

In some preferred embodiments, the oral dosage form of the invention provides an in-vitro release rate by weight of buprenorphine, when measured by the USP Basket or Paddle Method at 100 rpm in 900 mL aqueous buffer at a pH of between 1.6 and 7.2 at 37° C. of between 0% to about 100% at 1 hour, and greater than about 60% at 2 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing a mean in vitro controlled release rate of buprenorphine of 0.15 mg per hour to 35.0 mg per hour. In some preferred embodiments, when the oral dosage form is extended release and intended to provide a therapeutic effect of about 6, 8, 12 or 24 hours, said release rate is about 0.20 mg per hour to about 8.33 mg per hour, more preferably 0.42 mg per hour to 4.2 mg per hour, and most preferably 0.2 mg per hour to 2.5 mg per hour. In other preferred embodiments, the oral dosage form provides a therapeutic effect for about 6, 8, 12 or 24 hours and provide said release rate of about 0.20 mg per hour to about 8.33 mg per hour, more preferably 0.42 mg per hour to 4.2 mg per hour, and most preferably 0.2 mg per hour to 2.5 mg per hour.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% to about 40% at 1 hour, from about 5% to about 60% at 2 hours, from about 10% to about 75% at 4 hours, from about 20% to about 75% at 6 hours, from about 30% to about 80% at 9 hours, and greater than about 70% at 12 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 1% and about 45% at 1 hour, between about 5% and about 70% at 2 hours, between about 10% and about 90% at 4 hours, between about 20% and about 90% at 8 hours, greater than about 60% at 12 hours, greater than about 80% at 18 hours, and greater than about 85% at 24 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 5% and about 60% at 1 hour, between about 12.5% and about 80% at 2 hours, between about 25% and about 95% at 4 hours, between about 45% and about 100% at 8 hours, greater than about 55% at 12 hours, greater than about 65% at 18 hours, and greater than about 70% at 24 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 40% at 1 hour, between about 0% and about 70% at 2 hours, between about 5% and about 95% at 4 hours, between about 12.5% and about 100% at 8 hours, between about 20% and about 100% at 12 hours, between about 35% and about 100% at 16 hours, between about 55% and about 100% at 24 hours, and greater than about 75% at 36 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 60% at 1 hour, between about 0% and about 75% at 2 hours, between about 5% and about 95% at 4 hours, between about 12.5% and about 100% at 8 hours, between about 15% and about 100% at 12 hours, between about 25% to about 100% at 16 hours, between about 30% and about 100% hours at 24 hours and greater than 60% at 36 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release from the dosage form at one hour when measured by the USP Basket or Paddle Methods at 100 rpm in 700 ml of Simulated Gastric Fluid (SGF) at 37° C. of between 0% to about 50% by weight of buprenorphine. In other preferred embodiments, said release rate is between 0% to about 1%, or 0% to about 3%, or 0% to about 5%, or 0% to about 10%, or 0% to about 15%, or 0% to about 20%, 0% to about 30%, or 0% to about 40%, or 0% to about 60%, or 0% to about 70%, or 0% to about 80%, or 0% to about 90%, 0% to about 100%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release from the dosage form at one hour when measured by the USP Basket or Paddle Methods at 100 rpm in 900 mL aqueous buffer at a pH of between 1.6 and 7.2 at 37° C. of between 0% and about 60% at 1 hour, between about 0% and about 80% at 2 hours, between about 3% and about 95% at 4 hours and between about 10% and about 100% at 8 hours. In other preferred embodiments, said release rate is between 0% and about 10% at 1 hour, between about 0% and about 20% at 2 hours, between about 2% and about 80% at 4 hours and between about 5% and about 100% at 8 hours; or between 0% and about 20% at 1 hour, between about 0% and about 40% at 2 hours, between about 0% and about 80% at 4 hours and between about 2% and about 100% at 8 hours; or between 0% and about 40% at 1 hour, between about 0% and about 60% at 2 hours, between about 5% and about 85% at 4 hours and between about 5% and about 90% at 8 hours and greater than 20% at 12 hours; or between 0% and about 50% at 1 hour, between about 0% and about 50% at 2 hours, between about 10% and about 90% at 4 hours and between about 15% and about 90% at 8 hours and greater than 30% at 12 hours; or between 0% and about 70% at 1 hour, between about 0% and about 70% at 2 hours, between about 10% and about 75% at 4 hours and between about 15% and about 90% at 8 hours and greater than 30% at 12 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release from the dosage form at one hour when measured by the USP Basket or Paddle Methods at 100 rpm in 900 mL aqueous buffer at a pH of between 1.6 and 7.2 at 37° C. of between 10% and about 65% at 1 hour, between about 20% and about 75% at 2 hours, between about 30% and about 95% at 4 hours and between about 40% and about 100% at 8 hours. In other preferred embodiments, said release rate is between 2% and about 70% at 1 hour, between about 5% and about 80% at 2 hours, between about 10% and about 90% at 4 hours and between about 20% and about 100% at 8 hours; or between 5% and about 60% at 1 hour, between about 10% and about 75% at 2 hours, between about 15% and about 85% at 4 hours and between about 30% and about 100% at 8 hours; or between 20% and about 70% at 1 hour, between about 20% and about 75% at 2 hours, between about 20% and about 90% at 4 hours and between about 40% and about 100% at 8 hours; or between 30% and about 80% at 1 hour, between about 40% and about 85% at 2 hours, between about 40% and about 90% at 4 hours and between about 60% and about 100% at 8 hours; or between 1% and about 20% at 1 hour, between about 5% and about 20% at 2 hours, between about 10% and about 40% at 4 hours and between about 20% and about 40% at 8 hours and greater than 40% at 12 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% to about 47.5% at 1 hour, from about 10% to about 65% at 2 hours, from about 15% to about 70% at 4 hours, from about 25% to about 77.5% at 6 hours, from about 35% to about 87.5% at 9 hours, and greater than about 65% at 12 hours. In other preferred embodiments, said release rate is between 0% to about 30% at 1 hour, from about 5% to about 45% at 2 hours, from about 10% to about 60% at 4 hours, from about 15% to about 70% at 6 hours, from about 25% to about 80% at 9 hours, and greater than about 50% at 12 hours; or between 0% to about 20% at 1 hour, from about 2% to about 35% at 2 hours, from about 5% to about 50% at 4 hours, from about 10% to about 60% at 6 hours, from about 15% to about 70% at 9 hours, and greater than about 40% at 12 hours; or between 0% to about 10% at 1 hour, from about 1% to about 30% at 2 hours, from about 5% to about 40% at 4 hours, from about 10% to about 60% at 6 hours, from about 15% to about 70% at 9 hours, and greater than about 40% at 12 hours; or between 0% to about 5% at 1 hour, from about 0% to about 10% at 2 hours, from about 2% to about 20% at 4 hours, from about 5% to about 30% at 6 hours, from about 10% to about 40% at 9 hours, and greater than about 30% at 12 hours; or between 0% to about 50% at 1 hour, from about 15% to about 70% at 2 hours, from about 20% to about 75% at 4 hours, from about 30% to about 80% at 6 hours, from about 30% to about 90% at 9 hours, and greater than about 70% at 12 hours; or between 0% to about 60% at 1 hour, from about 15% to about 80% at 2 hours, from about 25% to about 85% at 4 hours, from about 35% to about 90% at 6 hours, from about 40% to about 90% at 9 hours, and greater than about 80% at 12 hours; or between 0% to about 70% at 1 hour, from about 20% to about 80% at 2 hours, from about 25% to about 80% at 4 hours, from about 35% to about 80% at 6 hours, from about 40% to about 80% at 9 hours, and greater than about 60% at 12 hours; or between 0% to about 75% at 1 hour, from about 30% to about 80% at 2 hours, from about 35% to about 90% at 4 hours, from about 50% to about 90% at 6 hours, from about 55% to about 95% at 9 hours, and greater than about 70% at 12 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 5% and about 50% at 1 hour, between about 10% and about 75% at 2 hours, between about 20% and about 95% at 4 hours, between about 40% and about 100% at 8 hours, greater than about 50% at 12 hours, greater than about 70% at 18 hours, and greater than about 80% at 24 hours. In other preferred embodiments, said release rate is between 2% and about 50% at 1 hour, between about 5% and about 75% at 2 hours, between about 15% and about 75% at 4 hours, between about 30% and about 90% at 8 hours, greater than about 40% at 12 hours, greater than about 60% at 18 hours, and greater than about 70% at 24 hours; or between 1% and about 40% at 1 hour, between about 2% and about 60% at 2 hours, between about 10% and about 65% at 4 hours, between about 20% and about 80% at 8 hours, greater than about 30% at 12 hours, greater than about 40% at 18 hours, and greater than about 60% at 24 hours; or between 5% and about 60% at 1 hour, between about 15% and about 80% at 2 hours, between about 25% and about 95% at 4 hours, between about 45% and about 100% at 8 hours, greater than about 60% at 12 hours, greater than about 80% at 18 hours, and greater than about 90% at 24 hours; or between 10% and about 65% at 1 hour, between about 20% and about 85% at 2 hours, between about 30% and about 100% at 4 hours, between about 60% and about 100% at 8 hours, greater than about 70% at 12 hours, greater than about 90% at 18 hours, and greater than about 95% at 24 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% to about 30% at 1 hour, from about 10% to about 65% at 4 hours, from about 20% to about 70% at 8 hours, from about 25% to about 80% at 12 hours, from about 35% to about 95% at 18 hours, and greater than about 65% at 24 hours. In other preferred embodiments, said release rate is between 0% to about 20% at 1 hour, from about 5% to about 50% at 4 hours, from about 10% to about 60% at 8 hours, from about 15% to about 70% at 12 hours, from about 25% to about 90% at 18 hours, and greater than about 55% at 24 hours; or between 0% to about 10% at 1 hour, from about 5% to about 40% at 4 hours, from about 8% to about 50% at 8 hours, from about 10% to about 60% at 12 hours, from about 22% to about 80% at 18 hours, and greater than about 45% at 24 hours; or between 0% to about 35% at 1 hour, from about 15% to about 70% at 4 hours, from about 25% to about 75% at 8 hours, from about 30% to about 85% at 12 hours, from about 40% to about 100% at 18 hours, and greater than about 75% at 24 hours; or between 0% to about 40% at 1 hour, from about 20% to about 70% at 4 hours, from about 30% to about 80% at 8 hours, from about 35% to about 90% at 12 hours, from about 45% to about 100% at 18 hours, and greater than about 80% at 24 hours; or between 0% to about 45% at 1 hour, from about 25% to about 75% at 4 hours, from about 35% to about 85% at 8 hours, from about 40% to about 90% at 12 hours, from about 50% to about 100% at 18 hours, and greater than about 90% at 24 hours; or between 0% to about 50% at 1 hour, from about 30% to about 80% at 4 hours, from about 40% to about 90% at 8 hours, from about 45% to about 95% at 12 hours, from about 60% to about 100% at 18 hours, and greater than about 95% at 24 hours; or between 0% to about 60% at 1 hour, from about 40% to about 80% at 4 hours, from about 45% to about 90% at 8 hours, from about 50% to about 100% at 12 hours, from about 70% to about 100% at 18 hours, and greater than about 80% at 24 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 50% at 1 hour, between about 0% and about 75% at 2 hours, between about 3% and about 95% at 4 hours, between about 10% and about 100% at 8 hours, between about 25% and about 100% at 12 hours, between about 30% and about 100% at 16 hours, between about 50% and about 100% at 24 hours, and greater than about 80% at 36 hours. In other preferred embodiments, said release rate is between 0% and about 40% at 1 hour, between about 0% and about 65% at 2 hours, between about 2% and about 85% at 4 hours, between about 8% and about 90% at 8 hours, between about 20% and about 95% at 12 hours, between about 25% and about 95% at 16 hours, between about 40% and about 90% at 24 hours, and greater than about 70% at 36 hours; or between 0% and about 30% at 1 hour, between about 0% and about 50% at 2 hours, between about 1% and about 75% at 4 hours, between about 5% and about 80% at 8 hours, between about 10% and about 85% at 12 hours, between about 15% and about 90% at 16 hours, between about 30% and about 80% at 24 hours, and greater than about 70% at 36 hours; or between 0% and about 60% at 1 hour, between about 0% and about 80% at 2 hours, between about 5% and about 100% at 4 hours, between about 15% and about 100% at 8 hours, between about 35% and about 100% at 12 hours, between about 40% and about 100% at 16 hours, between about 60% and about 100% at 24 hours, and greater than about 85% at 36 hours; or between 0% and about 65% at 1 hour, between about 0% and about 85% at 2 hours, between about 10% and about 100% at 4 hours, between about 20% and about 100% at 8 hours, between about 40% and about 100% at 12 hours, between about 50% and about 100% at 16 hours, between about 70% and about 100% at 24 hours, and greater than about 90% at 36 hours; or between 0% and about 70% at 1 hour, between about 0% and about 90% at 2 hours, between about 20% and about 100% at 4 hours, between about 30% and about 100% at 8 hours, between about 50% and about 100% at 12 hours, between about 60% and about 100% at 16 hours, between about 80% and about 100% at 24 hours, and greater than about 95% at 36 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 20% and about 50% at 1 hour, between about 40% and about 75% at 2 hours, between about 60% and about 95% at 4 hours, between about 80% and about 100% at 8 hours and between about 90% and about 100% at 12 hours. In other preferred embodiments, said release rate is between 15% and about 45% at 1 hour, between about 35% and about 70% at 2 hours, between about 55% and about 90% at 4 hours, between about 75% and about 90% at 8 hours and between about 80% and about 95% at 12 hours; or between 10% and about 40% at 1 hour, between about 30% and about 65% at 2 hours, between about 50% and about 85% at 4 hours, between about 70% and about 85% at 8 hours and between about 75% and about 90% at 12 hours; or between 5% and about 35% at 1 hour, between about 25% and about 60% at 2 hours, between about 45% and about 80% at 4 hours, between about 65% and about 80% at 8 hours and between about 70% and about 85% at 12 hours; or between 25% and about 55% at 1 hour, between about 45% and about 80% at 2 hours, between about 65% and about 95% at 4 hours, between about 85% and about 100% at 8 hours and between about 95% and about 100% at 12 hours; or between 30% and about 60% at 1 hour, between about 50% and about 80% at 2 hours, between about 70% and about 95% at 4 hours, between about 90% and about 100% at 8 hours and between about 95% and about 100% at 12 hours; or between 35% and about 60% at 1 hour, between about 50% and about 80% at 2 hours, between about 80% and about 95% at 4 hours, between about 90% and about 100% at 8 hours and between about 95% and about 100% at 12 hours; or between 20% and about 40% at 1 hour, between about 40% and about 65% at 2 hours, between about 60% and about 85% at 4 hours, between about 70% and about 90% at 8 hours and between about 80% and about 100% at 12 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 50% at 1 hour, between about 0% and about 75% at 2 hours, between about 10% and about 95% at 4 hours, between about 35% and about 100% at 8 hours, between about 55% and about 100% at 12 hours, between about 70% to about 100% at 16 hours, and greater than about 90% at 24 hours. In other preferred embodiments, said release rate is between 0% and about 40% at 1 hour, between about 0% and about 65% at 2 hours, between about 8% and about 85% at 4 hours, between about 30% and about 90% at 8 hours, between about 45% and about 100% at 12 hours, between about 60% to about 100% at 16 hours, and greater than about 80% at 24 hours; or between 0% and about 30% at 1 hour, between about 0% and about 55% at 2 hours, between about 5% and about 75% at 4 hours, between about 20% and about 80% at 8 hours, between about 35% and about 100% at 12 hours, between about 50% to about 100% at 16 hours, and greater than about 70% at 24 hours; or between 0% and about 20% at 1 hour, between about 0% and about 45% at 2 hours, between about 5% and about 65% at 4 hours, between about 10% and about 70% at 8 hours, between about 25% and about 80% at 12 hours, between about 40% to about 100% at 16 hours, and greater than about 60% at 24 hours; or between 0% and about 60% at 1 hour, between about 0% and about 80% at 2 hours, between about 15% and about 95% at 4 hours, between about 40% and about 100% at 8 hours, between about 60% and about 100% at 12 hours, between about 75% to about 100% at 16 hours, and greater than about 90% at 24 hours; or between 0% and about 65% at 1 hour, between about 0% and about 85% at 2 hours, between about 20% and about 90% at 4 hours, between about 45% and about 100% at 8 hours, between about 65% and about 100% at 12 hours, between about 80% to about 100% at 16 hours, and greater than about 90% at 24 hours; or between 0% and about 40% at 1 hour, between about 0% and about 50% at 2 hours, between about 10% and about 80% at 4 hours, between about 25% and about 70% at 8 hours, between about 40% and about 80% at 12 hours, between about 60% to about 100% at 16 hours, and greater than about 90% at 24 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 30% at 1 hour, between about 0% and about 45% at 2 hours, between about 3% and about 55% at 4 hours, between about 10% and about 65% at 8 hours, between about 20% and about 75% at 12 hours, between about 30% to about 88% at 16 hours, between about 50% and about 100% hours at 24 hours and greater than 80% at 36 hours. In other preferred embodiments, said release rate is between 0% and about 25% at 1 hour, between about 0% and about 40% at 2 hours, between about 2% and about 50% at 4 hours, between about 8% and about 60% at 8 hours, between about 10% and about 70% at 12 hours, between about 25% to about 80% at 16 hours, between about 45% and about 100% hours at 24 hours and greater than 75% at 36 hours; or between 0% and about 20% at 1 hour, between about 0% and about 35% at 2 hours, between about 1% and about 45% at 4 hours, between about 5% and about 55% at 8 hours, between about 8% and about 65% at 12 hours, between about 20% to about 75% at 16 hours, between about 40% and about 100% hours at 24 hours and greater than 70% at 36 hours; or between 0% and about 15% at 1 hour, between about 0% and about 30% at 2 hours, between about 0% and about 40% at 4 hours, between about 5% and about 50% at 8 hours, between about 8% and about 60% at 12 hours, between about 15% to about 70% at 16 hours, between about 35% and about 100% hours at 24 hours and greater than 60% at 36 hours; or between 0% and about 10% at 1 hour, between about 0% and about 25% at 2 hours, between about 0% and about 35% at 4 hours, between about 5% and about 45% at 8 hours, between about 10% and about 50% at 12 hours, between about 10% to about 60% at 16 hours, between about 30% and about 90% hours at 24 hours and greater than 70% at 36 hours; or between 0% and about 35% at 1 hour, between about 0% and about 50% at 2 hours, between about 5% and about 60% at 4 hours, between about 15% and about 70% at 8 hours, between about 25% and about 80% at 12 hours, between about 35% to about 90% at 16 hours, between about 55% and about 100% hours at 24 hours and greater than 85% at 36 hours; or between 0% and about 40% at 1 hour, between about 0% and about 55% at 2 hours, between about 10% and about 65% at 4 hours, between about 20% and about 75% at 8 hours, between about 30% and about 85% at 12 hours, between about 40% to about 100% at 16 hours, between about 55% and about 100% hours at 24 hours and greater than 90% at 36 hours; or between 0% and about 45% at 1 hour, between about 0% and about 60% at 2 hours, between about 15% and about 70% at 4 hours, between about 25% and about 80% at 8 hours, between about 35% and about 90% at 12 hours, between about 45% to about 100% at 16 hours, between about 60% and about 100% hours at 24 hours and greater than 60% at 36 hours; or between 0% and about 50% at 1 hour, between about 5% and about 65% at 2 hours, between about 20% and about 75% at 4 hours, between about 30% and about 85% at 8 hours, between about 40% and about 95% at 12 hours, between about 50% to about 100% at 16 hours, between about 70% and about 100% hours at 24 hours and greater than 70% at 36 hours; or between 0% and about 30% at 1 hour, between about 5% and about 40% at 2 hours, between about 10% and about 60% at 4 hours, between about 20% and about 70% at 8 hours, between about 30% and about 100% at 12 hours, between about 40% to about 100% at 16 hours, between about 60% and about 100% hours at 24 hours and greater than 90% at 36 hours; or between 0% and about 30% at 1 hour, between about 0% and about 30% at 2 hours, between about 0% and about 30% at 4 hours, between about 5% and about 70% at 8 hours, between about 10% and about 80% at 12 hours, between about 20% to about 100% at 16 hours, between about 40% and about 100% hours at 24 hours and greater than 50% at 36 hours; or between 0% and about 20% at 1 hour, between about 0% and about 20% at 2 hours, between about 0% and about 20% at 4 hours, between about 0% and about 20% at 8 hours, between about 5% and about 40% at 12 hours, between about 10% to about 80% at 16 hours, between about 40% and about 100% hours at 24 hours and greater than 60% at 36 hours; or between 0% and about 10% at 1 hour, between about 0% and about 20% at 2 hours, between about 0% and about 40% at 4 hours, between about 5% and about 60% at 8 hours, between about 10% and about 80% at 12 hours, between about 20% to about 100% at 16 hours, between about 40% and about 100% hours at 24 hours and greater than 50% at 36 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 0% and about 50% at 1 hour, between about 0% and about 75% at 2 hours, between about 3% and about 95% at 4 hours, between about 10% and about 100% at 8 hours, between about 20% and about 100% at 12 hours, between about 30% to about 100% at 16 hours, between about 50% and about 100% hours at 24 hours and greater than 80% at 36 hours. In other preferred embodiments, said release rate is between 0% and about 45% at 1 hour, between about 0% and about 70% at 2 hours, between about 3% and about 90% at 4 hours, between about 8% and about 100% at 8 hours, between about 15% and about 100% at 12 hours, between about 25% to about 100% at 16 hours, between about 45% and about 100% hours at 24 hours and greater than 80% at 36 hours; or between 0% and about 40% at 1 hour, between about 0% and about 65% at 2 hours, between about 0% and about 80% at 4 hours, between about 5% and about 80% at 8 hours, between about 10% and about 90% at 12 hours, between about 20% to about 100% at 16 hours, between about 40% and about 100% hours at 24 hours and greater than 70% at 36 hours; or between 0% and about 35% at 1 hour, between about 0% and about 60% at 2 hours, between about 0% and about 70% at 4 hours, between about 3% and about 70% at 8 hours, between about 5% and about 80% at 12 hours, between about 15% to about 100% at 16 hours, between about 30% and about 100% hours at 24 hours and greater than 40% at 36 hours; or between 0% and about 60% at 1 hour, between about 0% and about 80% at 2 hours, between about 5% and about 100% at 4 hours, between about 15% and about 100% at 8 hours, between about 30% and about 100% at 12 hours, between about 40% to about 100% at 16 hours, between about 60% and about 100% hours at 24 hours and greater than 70% at 36 hours; or between 0% and about 50% at 1 hour, between about 0% and about 75% at 2 hours, between about 5% and about 95% at 4 hours, between about 25% and about 80% at 8 hours, between about 30% and about 100% at 12 hours, between about 40% to about 100% at 16 hours, between about 60% and about 100% hours at 24 hours and greater than 60% at 36 hours; or between 0% and about 60% at 1 hour, between about 0% and about 85% at 2 hours, between about 5% and about 100% at 4 hours, between about 10% and about 100% at 8 hours, between about 20% and about 100% at 12 hours, between about 30% to about 100% at 16 hours, between about 50% and about 100% hours at 24 hours and greater than 80% at 36 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing an in-vitro release rate by weight of buprenorphine, when measured by the USP Basket or Paddle Methods at 100 rpm in 900 mL aqueous buffer at a pH of between 1.6 and 7.2 at 37° C. of between 15% and about 25% at 1 hour, between about 25% and about 35% at 2 hours, between about 30% and about 45% at 4 hours, between about 40% and about 60% at 8 hours, between about 55% and about 70% at 12 hours and between about 60% to about 75% at 16 hours. In other preferred embodiments, said release rate is between 10% and about 20% at 1 hour, between about 20% and about 30% at 2 hours, between about 25% and about 40% at 4 hours, between about 30% and about 50% at 8 hours, between about 50% and about 65% at 12 hours and between about 55% to about 65% at 16 hours; or between 5% and about 15% at 1 hour, between about 15% and about 25% at 2 hours, between about 20% and about 35% at 4 hours, between about 25% and about 45% at 8 hours, between about 45% and about 60% at 12 hours and between about 50% to about 60% at 16 hours; or between 15% and about 30% at 1 hour, between about 20% and about 40% at 2 hours, between about 20% and about 50% at 4 hours, between about 30% and about 70% at 8 hours, between about 60% and about 80% at 12 hours and between about 70% to about 90% at 16 hours; or between 0% and about 50% at 1 hour, between about 5% and about 50% at 2 hours, between about 5% and about 70% at 4 hours, between about 10% and about 80% at 8 hours, between about 20% and about 100% at 12 hours and between about 40% to about 100% at 16 hours; or between 15% and about 40% at 1 hour, between about 15% and about 45% at 2 hours, between about 20% and about 60% at 4 hours, between about 20% and about 80% at 8 hours, between about 30% and about 90% at 12 hours and between about 40% to about 100% at 16 hours.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said in-vitro release rate being substantially independent of pH in that a difference, at any given time, between an amount of said buprenorphine released at one pH and an amount released at any other pH, when measured in-vitro using the USP Basket or Paddle Methods of USP Drug Release test of U.S. Pharmacopeia (2003) at 100 rpm in 900 ml aqueous buffer, is no greater than 30%. In other preferred embodiments, the difference, at any given time, between an amount of buprenorphine released at one pH and an amount released at any other pH using the aforementioned methods is no greater than 50%, or no greater than 40%, or no greater than 35%, or no greater than 25%, or no greater than 20%, or no greater than 15%, or no greater than 10%, or no greater than 5%.

Although dosage forms that provide pH independent in vitro dissolution and in vivo release are frequently sought after and viewed favorably, particularly many controlled release or extended release forms, in some embodiments, pH independent dissolution and release can work against the objectives of the some oral dosage forms of buprenorphine (delayed onset, rapid release or delayed onset, extended release, or delayed onset, pulsatile release) which are intended to provide delivery or release of the dosage form in the proximal to the stomach, duodenum, or ileum (i.e., duodenal release, jejunal release, ileal release, ileo-colonic release or colonic release). Indeed, certain controlled release material used to achieve delayed onset, duodenal release, jejunal release, ileal release, ileo-colonic release or colonic release exploit the pH difference in the GI tract to achieve some or all of its objectives.

Therefore, in some preferred embodiments, where the oral buprenorphine pharmaceutical composition is intended to provide delayed onset, rapid release or delayed onset, extended release, or delayed onset, pulsatile release through use of a pH sensitive controlled release material, the in-vitro release rate is substantially dependent on pH in that the amount of buprenorphine released at an undesirable pH (e.g., pH 1.2) and the amount released at a desirable pH (e.g., depending on the controlled release material and delivery and release objectives, pH 5.5, 6, 6.5, 7, 7.2, 7.4), when measured in-vitro at 1.5 or 2 hours using the USP Basket or Paddle Method of USP Drug Release test of U.S. Pharmacopeia (2003) at 100 rpm in 900 ml aqueous buffer is significantly different. For example, in some preferred embodiments, the in-vitro release rate difference is greater than about 35%, or 40%, or 45%, or 50%, or 55%, or 60% or 70%, or 80%, with the release rate higher at the desirable pH compared with the undesirable pH.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said in-vitro release rate being substantially independent of pH in that a difference, at any given time, between an amount of said buprenorphine released at one pH and an amount released at any other pH, when measured in-vitro using the USP Basket or Paddle Methods of USP Drug Release test of U.S. Pharmacopeia (2003) at 100 rpm in 900 ml aqueous buffer, is no greater than 30%. In other preferred embodiments, the difference, at any given time, between an amount of buprenorphine released at one pH and an amount released at any other pH using the aforementioned methods is no greater than 50%, or no greater than 40%, or no greater than 35%, or no greater than 25%, or no greater than 20%, or no greater than 15%, or no greater than 10%, or no greater than 5%.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage form providing in-vitro release rates by weight of between 0% to about 50% by weight of buprenorphine from the dosage form at one hour when measured by the USP Basket or Paddle Methods at 100 rpm in 700 ml of Simulated Gastric Fluid (SGF) at 37° C. In other preferred embodiments, said release rate at one hour is between 0% to about 10% by weight, or 0% to about 20% by weight, or is between 0% to about 30% by weight, or 0% to about 40% by weight, or between 0% to about 60% by weight, or 0% to about 70% by weight, or 0% to about 80% by weight, or 0% to about 90% by weight, or 10% to about 50% by weight, or 10% to about 60% by weight, or 10% to about 70% by weight, or 10% to about 90% by weight, or 10% to about 100% by weight, or 30% to about 100% by weight, or 50% to about 100% by weight.

In some preferred embodiments, the dosage form provides an oral pharmaceutical composition comprising a therapeutically effective amount of buprenorphine, said dosage form suitable for four times-a-day (Q6H or Q6H PRN), three times-a-day (Q8H or Q8H PRN), twice-a-day (Q12H or Q12H PRN) or once-a-day (QD, Q24H or Q24H PRN) administration to a human patient, said dosage forms providing in-vitro release rates by weight of buprenorphine, when measured by the USP Basket or Paddle Methods at 100 rpm in 900 mL aqueous buffer at a pH of between 1.6 and 7.2 at 37° C. of between 0% to about 80% at 0.5 hours, and greater than about 40% at 1 hour. In other preferred embodiments, said release rate is between 0% to about 40% at 0.5 hours, and greater than about 60% at 1 hour; or between 0% to about 20% at 0.5 hours, and greater than about 40% at 1 hour; or between 0% to about 20% at 0.5 hours, and greater than about 20% at 1 hour; or between 0% to about 90% at 0.5 hours, and greater than about 60% at 1 hour; or between 0% to about 100% at 0.5 hours, and greater than about 60% at 1 hour; or between 0% to about 90% at 1 hour, and greater than about 40% at 2 hours; or between 0% to about 100% at 1 hour, and greater than about 60% at 2 hours; or between 0% to about 60% at 1 hour, and greater than about 40% at 2 hours; or between 0% to about 40% at 1 hour, and greater than about 30% at 2 hours; or between 0% to about 50% at 1 hour, and greater than about 40% at 2 hours; or between 0% to about 30% at 1 hour, and greater than about 20% at 2 hours; or between 0% and about 50% at 1 hour, between about 0% and about 80% at 2 hours, between about 5% and about 100% at 4 hours and between about 10% and about 100% at 8 hours; or between 10% and about 60% at 1 hour, between about 15% and about 75% at 2 hours, between about 20% and about 95% at 4 hours and between about 30% and about 100% at 8 hours.

In certain embodiments, the dosage forms of the invention contains one or more substances in sufficient quantity to render said dosage form controlled release, such that: (i) in-vitro release rate of buprenorphine by weight of any commercially available sublingual formulations of buprenorphine at the time of this invention (e.g., as listed in the FDA's Orange Book, the EMEA website, Martindale: The Complete Drug Reference, 35th Edition, Pharmaceutical Press) compared with in-vitro release rate by weight of buprenorphine from the dosage form of the invention is at least about 10%, or at least about 15%, or at least about 20%, or at least about 30%, or at least about 40%, or at least about 50%, or at least about 60%, or at least about 70%, or at least about 80%, or at least about 90% faster, or at least 100% faster, or at least 200% faster, or at least 300% faster, or at least 400% faster, or at least 500% faster, or at least 600% faster, or at least 700% faster, or at least 800% faster, or at least 1000% faster, at 0.25 hour, or at 0.5 hours, or at 1 hour, or at 2 hours, or at 1 to 2 hours, when measured by the USP Basket or Paddle Methods at 100 rpm in 900 mL aqueous buffer at a pH of between 1.6 and 7.2 at 37° C.; and/or (ii) in-vivo release rate of buprenorphine by weight of any commercially available sublingual formulations of buprenorphine at the time of this invention (e.g., as listed in the FDA's Orange Book, the EMEA website, Martindale: The Complete Drug Reference, 35th Edition, Pharmaceutical Press) compared with the in-vivo release rate of buprenorphine from the dosage form of the invention is at least about 10%, or at least about 15%, or at least about 20%, or at least about 30%, or at least about 40%, or at least about 50%, or at least about 60%, or at least about 70%, or at least about 80%, or at least about 90% faster, or at least 100% faster, or at least 200% faster, or at least 300% faster, or at least 400% faster, or at least 500% faster, or at least 600% faster, or at least 700% faster, or at least 800% faster, or at least 1000% faster, said in-vivo release rate quantified by the Cₘₐₓ of buprenorphine after single dose administration to human subjects, each dosage form administered according to its approved route of administration.

In some preferred embodiments, the oral dosage form is a controlled release material suitable for extended release oral administration to a human patient of the dosage form comprises a matrix. In some preferred embodiments, the said matrix is a plurality of multiparticulate matrices. In some preferred embodiments, the multiparticulates are compressed into a tablet. In some preferred embodiments, the multiparticulates are disposed in a pharmaceutically acceptable capsule.

In some preferred embodiments, the in vivo pharmacokinetic parameters of the specifications and claims are derived or determined from first administration. In other preferred embodiments, the in vivo pharmacokinetic parameters are derived or determined from steady state administration.

In some preferred embodiments, the in vivo pharmacokinetic parameters of the specifications and claims are derived or determined under fed conditions. In other preferred embodiments, the in vivo pharmacokinetic parameters are derived or determined under fasted conditions.

In some preferred embodiments, the in vivo pharmacokinetic parameters of the specifications and claims are derived or determined from an individual subject. In other preferred embodiments, the in vivo pharmacokinetic parameters are derived or determined from a population of subjects.

In some preferred embodiments, the in vivo specifications and claims of the invention are measured, reported, observed or achieved after administration of some or most doses of the invention. In other preferred embodiments, the in vivo specifications and claims of the invention are measured, reported, observed or achieved after administration of substantially all or all doses of the invention.

In some preferred embodiments, the in vivo pharmacokinetic parameters of the specifications and claims are derived or determined in subjects having a Body Mass Index (BMI) between 18 and 26 kg/^{m2}, inclusive (BMI=[weight in kg/height in ^{m2}]×10,000). In some preferred embodiments, the in vivo pharmacokinetic parameters of the specifications and claims are derived or determined in subjects having a Body Mass Index (BMI)≧38 kg/^{m2}.

Also disclosed are methods for the targeted release of buprenorphine from the dosage form into the duodenum, jejunum, ileum and colon to provide a therapeutic effect comprising administering a therapeutically effective amount of oral buprenorphine or a pharmaceutically acceptable salt of buprenorphine or a mixture thereof.

Also disclosed are methods for the treatment of opioid dependence or addiction disorders in a human patient suffering comprising administering a therapeutically effective amount of oral buprenorphine or a pharmaceutically acceptable salt of buprenorphine or a mixture thereof. Preferably, the addiction disorder is an opioid addiction disorder or a poly-substance abuse disorder.

Also disclosed are methods for resisting, deterring, minimizing or preventing drug abuse, drug diversion and drug addiction in a human patient comprising administering a therapeutically effective amount of oral buprenorphine or a pharmaceutically acceptable salt of buprenorphine or a mixture thereof.

Also disclosed are methods for the treatment of medical conditions amenable to treatment with buprenorphine in patients who are at higher risk for nausea, vomiting, sedation or other opioid agonist side effects or who have a prior history of said side effects on other opioids comprising administering a therapeutically effective amount of oral buprenorphine or a pharmaceutically acceptable salt of buprenorphine or a mixture thereof.

Also disclosed are methods for the treatment of medical conditions amenable to treatment with buprenorphine in patients who are at higher risk for nausea, vomiting, sedation or other side effects with sublingual buprenorphine or oral immediate release buprenorphine comprising administering a therapeutically effective amount of oral buprenorphine or a pharmaceutically acceptable salt of buprenorphine or a mixture thereof.

Also disclosed are methods for the treatment of dyspnea, cough and COPD comprising administering a therapeutically effective amount of oral buprenorphine or a pharmaceutically acceptable salt of buprenorphine or a mixture thereof.

Also disclosed are methods for the treatment of medical conditions amenable to treatment with buprenorphine or opioid agonists in a human patient who also suffers from an addiction disorder, who is at risk or increased risk for addiction or who may be prone to drug diversion into illicit channels comprising administering a therapeutically effective amount of oral buprenorphine or a pharmaceutically acceptable salt of buprenorphine or a mixture thereof.

Also disclosed are methods for the treatment of medical conditions amenable to treatment with buprenorphine or opioid agonists in a human patient suffering comprising administering a therapeutically effective amount of oral buprenorphine or a pharmaceutically acceptable salt of buprenorphine or a mixture thereof.

Also disclosed are methods for preventing and treating pain in a human patient comprising administering a therapeutically effective amount of oral buprenorphine or a pharmaceutically acceptable salt of buprenorphine or a mixture thereof.

All pain states are contemplated by this invention, regardless of etiology, mechanisms, duration, prior treatment response and anatomic location, including acute pain, inflammatory pain, chronic pain, cancer pain, visceral pain and neuropathic pain.

Also disclosed are methods of providing relief in a human patient suffering from neuropathic and chronic pain comprising a therapeutically effective amount of oral buprenorphine or a pharmaceutically acceptable salt buprenorphine or a mixture thereof. In some preferred embodiments, the dosage form of the invention is intended for the treatment of neuropathic pain, peripheral neuropathic pain, central neuropathic pain, chronic pain, osteoarthritis, back pain, cancer pain, and chronic inflammatory pain.

Also disclosed are methods of providing relief in a human patient suffering from acute pain comprising a therapeutically effective amount of oral buprenorphine or a pharmaceutically acceptable salt buprenorphine or a mixture thereof.

Also disclosed are methods of providing relief in a human patient suffering from an addiction disorder comprising a therapeutically effective amount of oral buprenorphine or a pharmaceutically acceptable salt buprenorphine or a mixture thereof.

All kinds of kits of the present invention are contemplated. In some preferred embodiments, also provided are kits for use in treating or preventing the pain with the oral administration of buprenorphine or a pharmaceutically acceptable salt of buprenorphine, or a mixture thereof for a subject in need of such treatment, comprising: (i) a dosage form of the invention; (ii) a container for the dosage form; and optionally, any of (iii) to (vi): (iii) a container for individual units of the dosage form (e.g., individual tablets or capsules in blisters); (iv) educational instructions in any media about various medical conditions, their etiology, pathophysiology, consequences and treatment, including information on the potential for abuse and diversion and methods for prevention of same and information on the proper use and disposal of the medication; (v) containers or bags for the safe disposal of any used or remaining unused dosage form, preferably child proof and flushable; (vi) tamper evident and child proof packaging for the kit and its contents.

The amount of buprenorphine in the oral dosage form will vary depending on variety of physiologic, pharmacologic, pharmacokinetic, pharmaceutical and physicochemical factors, including: (i) the choice of buprenorphine as the base, pharmaceutically acceptable salt or mixtures thereof; (ii) the nature of the oral dosage form (e.g, immediate release or extended release); (iii) the anatomical location of the pain relieving target; (iv) the intensity and intractability of the pain; (v) the contribution of different mechanism to the initiation, propagation, summation and maintenance of the pain; (vi) the absorption, metabolism, distribution and excretion of orally administered buprenorphine in healthy subjects and in patients with various diseases and disorders, including renal and hepatic impairment; (vii) the presence of comorbid pathology; (viii) the patient's risk of iatrogenic side effects; (ix) the tolerability of the dose, including the patient's propensity for buprenorphine associated CNS and gastrointestinal side effects; (x) use of concurrent analgesics; (xi) the efficiency of the dosage form.

In certain embodiments, the amount of buprenorphine in the dosage form is about 0.001 mg to 1500 mg. In other embodiments, the amount of buprenorphine in the dosage form is about 0.1 mg to 1000 mg. In other embodiments, the amount of buprenorphine in the dosage form is about 0.5 mg to about 500 mg or about 1 mg to about 200 mg, or 2 mg to about 100 mg or 1 mg to about 60 mg. In certain embodiments, the maximum dose of oral buprenorphine exceeds the maximum approved dose of sublingual buprenorphine by at least about 5%, or 10%, or 15%, or 20%, or 30%, or 40%, or 50%, or 60%, or 70%, or 80%, or 90%, or 100%, or 120%, or 140%, or 160%, or 180%, or 200%, or 220%, or 240%, or 260%, or 280%, or 300%, or 320%, or 340%, or 360%, or 380%, or 400%, or 450%, or 500%, or 550%, or 600%, or 650%, or 700%. In certain embodiments, the minimum dose of oral buprenorphine exceeds the minimum approved dose of sublingual buprenorphine by at least about 5%, or 10%, or 15%, or 20%, or 30%, or 40%, or 50%, or 60%, or 70%, or 80%, or 90%, or 100%, or 120%, or 140%, or 160%, or 180%, or 200%, or 220%, or 240%, or 260%, or 280%, or 300%, or 320%, or 340%, or 360%, or 380%, or 400%, or 450%, or 500%, or 550%, or 600%, or 650%, or 700%. In certain embodiments, the average dose of oral buprenorphine exceeds the average dose of sublingual buprenorphine by at least about 5%, or 10%, or 15%, or 20%, or 30%, or 40%, or 50%, or 60%, or 70%, or 80%, or 90%, or 100%, or 120%, or 140%, or 160%, or 180%, or 200%, or 220%, or 240%, or 260%, or 280%, or 300%, or 320%, or 340%, or 360%, or 380%, or 400%, or 450%, or 500%, or 550%, or 600%, or 650%, or 700%. In certain embodiments, the induction (where appropriate) or maintenance dose of oral buprenorphine exceeds the approved induction (where appropriate) or maintenance dose of sublingual buprenorphine by at least about 5%, or 10%, or 15%, or 20%, or 30%, or 40%, or 50%, or 60%, or 70%, or 80%, or 90%, or 100%, or 120%, or 140%, or 160%, or 180%, or 200%, or 220%, or 240%, or 260%, or 280%, or 300%, or 320%, or 340%, or 360%, or 380%, or 400%, or 450%, or 500%, or 550%, or 600%, or 650%, or 700%. In certain embodiments, the induction (where appropriate) dose, maintenance dose, minimum dose, average doe and/or maximum dose of oral buprenorphine exceeds the corresponding dose sublingual buprenorphine by at least about 5%, or 10%, or 15%, or 20%, or 30%, or 40%, or 50%, or 60%, or 70%, or 80%, or 90%, or 100%, or 120%, or 140%, or 160%, or 180%, or 200%, or 220%, or 240%, or 260%, or 280%, or 300%, or 320%, or 340%, or 360%, or 380%, or 400%, or 450%, or 500%, or 550%, or 600%, or 650%, or 700%. In certain embodiments, the dose of oral buprenorphine is at least about 2 mg, or 3 mg, or 4 mg, or 5 mg, or 6 mg, or 7 mg, or 8 mg, or 9 mg, or 10 mg or 11 mg, or 12 mg, or 13 mg, or 14 mg, or 15 mg, or 16 mg, or 17 mg, or 18 mg, or 19 mg, or 20 mg, or 21 mg, or 22 mg, or 23 mg, or 24 mg, or 25 mg, or 26 mg, or 27 mg, or 28 mg, or 29 mg, or 30 mg, or 32 mg, or 34 mg, or 35 mg, or 36 mg, or 38 mg, or 40 mg, or 45 mg, or 50 mg, or 55 mg, or 60 mg, or 65 mg, or 70 mg. In certain embodiments, the amount of buprenorphine base in the dosage form is not less than 10 mg, or not less than about 10 mg, or 11 mg, or 12 mg, or 13 mg or 14 mg, or 15 mg, or 16 mg, or 17 mg, or 18 mg, or 19 mg, or 20 mg, or 21 mg, or 22 mg, or 23 mg, or 24 mg, or 25 mg, or 26 mg, or 27 mg, or 28 mg, or 29 mg, or 30 mg. In certain embodiments, the amount of buprenorphine hydrochloride in the dosage form is not less than 10 mg, or not less than about 10 mg, or 11 mg, or 12 mg, or 13 mg or 14 mg, or 15 mg, or 16 mg, or 17 mg, or 18 mg, or 19 mg, or 20 mg, or 21 mg, or 22 mg, or 23 mg, or 24 mg, or 25 mg, or 26 mg, or 27 mg, or 28 mg, or 29 mg, or 30 mg. In certain embodiments, the amount of any pharmaceutically acceptable salt of buprenorphine in the dosage form is not less than 10 mg, or not less than about 10 mg, or 11 mg, or 12 mg, or 13 mg or 14 mg, or 15 mg, or 16 mg, or 17 mg, or 18 mg, or 19 mg, or 20 mg, or 21 mg, or 22 mg, or 23 mg, or 24 mg, or 25 mg, or 26 mg, or 27 mg, or 28 mg, or 29 mg, or 30 mg. In certain embodiments, the daily dose of buprenorphine or its pharmaceutically acceptable salt in the dosage form is not less than 40 mg, or 50 mg or 60 mg or 65 mg.

The invention is also directed to methods of preparing the dosage forms disclosed herein.

The invention is also directed to a process for the preparation and manufacture of the dosage form.

The invention is also directed to methods of treating buprenorphine responsive medical conditions comprising administering a therapeutically effective amount of oral buprenorphine in a dosage form of the invention, or pharmaceutically acceptable salts thereof or mixtures thereof.

The invention is also directed to methods of treating pain, chronic pain, neuropathic pain, opioid dependence, dyspnea, cough and addiction disorders comprising administering a therapeutically effective amount of oral buprenorphine in a dosage form of the invention, or pharmaceutically acceptable salts thereof or mixtures thereof.

The invention is also directed to methods of treating buprenorphine responsive medical conditions with reduced risk of drug abuse, drug misuse, and drug diversion comprising administering a therapeutically effective amount of oral buprenorphine in a dosage form of the invention, or pharmaceutically acceptable salts thereof or mixtures thereof.

The invention is also directed to methods of improving treatment compliance and deter episodic, occasional, intermittent, periodic, as needed, or PRN use of the dosage form when treating buprenorphine responsive medical conditions requiring more than few days of therapy to more than a few weeks of therapy or chronic therapy comprising administering a therapeutically effective amount of oral buprenorphine in a dosage form of the invention, or pharmaceutically acceptable salts thereof or mixtures thereof.

In certain preferred embodiments, the buprenorphine in the dosage form is combined with one or more other drugs for the treatment of the same medical condition as the buprenorphine or for the treatment of a different medical condition. All modes of co-administration are contemplated, including via an oral, subcutaneous, direct intravenous, slow intravenous infusion, continuous intravenous infusion, intravenous or epidural patient controlled analgesia (PCA and PCEA), intramuscular, intrathecal, epidural, intracisternal, intramuscular, intraperitoneal, transdermal, topical, transmucosal, buccal, sublingual, inhalation, intranasal, epidural, intra-atricular, intranasal, rectal or ocular routes.

The term “first administration” means administration of a dose of the present invention at the initiation of therapy to an individual patient or a patient population.

The term “steady state” means that the amount of the drug reaching the system is approximately the same as the amount of the drug leaving the system. Thus, at “steady-state”, the patient's body eliminates the drug at approximately the same rate that the drug becomes available to the patient's system through absorption into the blood stream.

As used herein the terms: (i) “AUC₀₋ₜ”, and “AUC_{0-τ” (or “AUC}_{0-Tau}”) mean the area under the plasma drug concentration-time curve from time zero to the intended dosing frequency of the dosage form after a single administration (e.g., 8 hours, 12 hours or 24 hours) and to the end of the dosing interval after repeated dosing or at steady-state, respectively; (ii) “AUC_{0-inf}” (means area under the plasma drug concentration-time curve from time zero to infinity; (iii) “AUC₀₋₆” means area under the plasma drug concentration-time curve from time zero to 6 hours after dosing; (iv) “AUC₀₋₈” means area under the plasma drug concentration-time curve from time zero to 8 hours after dosing; (v) “AUC₀₋₁₂” means area under the plasma drug concentration-time curve from time zero to 12 hours after dosing; (vi) “AUC₀₋₂₄” means area under the plasma drug concentration-time curve from time zero to 24 hours after dosing; (vii) “Cₘₐₓ” means the maximum observed plasma drug concentration; (viii) “C₆” means the plasma drug concentration at 6 hours after dosing; (ix) “C₈” means the plasma drug concentration at 8 hours after dosing; (x) “C₁₂” means the plasma drug concentration at 12 hours after dosing; (xi) “C₂₄” means the plasma drug concentration at 24 hours after dosing; (xii) “tₘₐₓ” or “Tₘₐₓ” means the time of the observed maximum drug concentration (also known as time to achieve Cₘₐₓ); (xiii) “Cₘᵢₙ” means the minimum observed drug concentration following the maximum plasma concentration or the concentration at the end of the intended dosing interval; (xiv) “half value duration” or “HVD” means the duration over the dosing interval during which plasma concentration of drug are greater than or equal to one-half of Cₘₐₓ, obtained by calculating the time interval beginning when the interpolated concentration first equals or exceeds one-half of Cₘₐₓ and ending at the first time point for which the interpolated concentration falls below one-half of Cₘₐₓ; (xv) “W₅₀” for purposes of the present invention means the width of the plasma concentration time curve at 50% of the height of the Cₘₐₓ over the dosing interval; (xvi) “steady state” is a state of equilibrium wherein the amount of the drug reaching the system is approximately the same as the amount of the drug leaving the system or put another way, the patient's body eliminates the drug at approximately the same rate that the drug becomes available to the patient's system through absorption into the blood stream, said “time to steady state” measured by calculating the Cₘᵢₙ after each sequential dosing of drug administered at the intended dosing frequency until two consecutive Cₘᵢₙ's are not statistically different at a 10% significance level (p=0.10); (xvii) “percent fluctuation” means the variation in plasma concentrations of the drug computed as: (a) (Cₘₐₓ−Cₘᵢₙ)/Cₘᵢₙ×100 (for an individual patient) and (mean Cₘₐₓ−mean Cₘᵢₙ)/mean Cₘᵢₙ×100 (for a population); or (b) (Cₘₐₓ−Cₘᵢₙ)/Cₐᵥ×100 (for an individual patient) and (mean Cₘₐₓ−mean Cₘᵢₙ)/mean Cₐᵥ×100 (for a population); (xviii) “accumulation index” or “AI” means the ratio of the plasma concentration of the drug at the end of the intended dosing interval (i.e., 8 hours for a Q8H dosage form, 12 hours for a Q12H dosage form, and 24 hours for a Q24H dosage form) after administration, determined at steady-state (Cₛₛₘᵢₙ) to the plasma concentration of the drug at the end of the intended dosing interval determined at first administration (i.e., after the first dose); (xix) “AUC₀₋ₙ” means the area under the plasma drug concentration-time curve from time zero to the specified time point (“n).

Pharmacokinetic parameters of the invention are be computed from single dose (i.e., first administration) and steady state pharmacokinetic studies conducted in an individual subject or in a population of subjects in the fasted or fed states. The AI and percent of steady state computations requires both single dose (i.e., first administration) and steady state pharmacokinetic assessment.

In certain preferred embodiments of the present invention, an effective amount of buprenorphine in immediate release form is included in the controlled release unit dose buprenorphine formulation to be administered. The immediate release form of the buprenorphine is preferably included in an amount which is effective to shorten the time to Cₘₐₓ of the buprenorphine in the blood (e.g., plasma). In such embodiments, an effective amount of the buprenorphine in immediate release form may be coated onto the substrates of the present invention. For example, where the extended release buprenorphine from the formulation is due to a controlled release coating, the immediate release layer would be overcoated on top of the controlled release coating. On the other hand, the immediate release layer maybe coated onto the surface of substrates wherein the buprenorphine is incorporated in a controlled release matrix. Where a plurality of the sustained release substrates comprising an effective unit dose of the buprenorphine (e.g., multiparticulate systems including pellets, spheres, beads and the like) are incorporated into a hard gelatin capsule, the immediate release portion of the buprenorphine dose may be incorporated into the gelatin capsule via inclusion of the sufficient amount of immediate release buprenorphine as a powder or granulate within the capsule. Alternatively, the gelatin capsule itself may be coated with an immediate release layer of the buprenorphine. One skilled in the art would recognize still other alternative manners of incorporating the immediate release buprenorphine into the unit dose. Such alternatives are deemed to be encompassed by the appended claims. By including such an effective amount of immediate release buprenorphine in the unit dose, the experience of relatively higher levels of pain in patients may be significantly reduced.

For purposes of the invention, the term “a patient” in reference to pharmacokinetic parameters means that the discussion (or claim) is directed to the pharmacokinetic parameters of an individual patient or subject.

The term “population of patients” or “patient population” means that the discussion (or claim) is directed to the mean pharmacokinetic parameters of at least two patients or subjects.

In certain preferred embodiments, any one or all of the above in-vivo parameters are achieved after a first administration (often referred to as “single dose administration”) of the dosage form to a human patient or a population of human patients.

In certain alternative embodiments, any one or all of the above in-vivo parameters are achieved after steady state administration of the dosage form to a human patient or a population of human patients.

The term “USP Paddle or Basket Method” is the Paddle and Basket Method described, e.g., in specified in the United States Pharmacopeia, USP-28 NF-23 (2005), published by the United States Pharmacopeial Convention, Inc, herein incorporated by reference.

The term “pH-dependent” for purposes of the present invention is defined as having characteristics (e.g., dissolution) which vary according to environmental pH.

The term “pH-independent” for purposes of the present invention is defined as having characteristics (e.g., dissolution) which are substantially unaffected by pH.

The term “pH-dependent” for purposes of the present invention is defined as having characteristics (e.g., dissolution) which are substantially affected by pH.

The term “bioavailability” is defined for purposes of the present invention as the extent to which the drug (e.g., buprenorphine) is absorbed from the unit dosage forms.

All oral pharmaceutical dosage forms of the invention are contemplated, including oral suspensions, tablets, capsules, effervescent tablets, effervescent powders, powders, solutions, powders for reconstitution, oral gastroretentive tablets and capsules, administered as immediate release, modified release, enteric coated, sustained release, controlled release, pulsatile release and extended release dosage form.

In some preferred embodiments of the invention, the dosage form comprises one or more of the following: modified release or enteric coated or sustained release or controlled release or pulsatile release or extended release. In some preferred embodiments of the invention, the dosage form comprises only one of the following: modified release or enteric coated or sustained release or controlled release or pulsatile release or extended release. In some preferred embodiments of the invention, the dosage form specifically excludes one or more of the following: modified release or enteric coated or sustained release or controlled release or pulsatile release or extended release.

In some embodiments, the invention specifically excludes oral immediate release dosage forms.

In some preferred embodiments of the invention, the dosage form of the invention is controlled release, extended release, sustained release, modified release, or delayed onset.

In some preferred embodiments of the invention, the dosage form of the invention is delayed onset, rapid release or delayed onset, extended release, or delayed onset, pulsatile release.

In some preferred embodiments of the invention, the dosage form of the invention is delayed onset, duodenal delivery, jejunal delivery, ileal delivery, ileo-colonic delivery, colonic delivery

In some embodiments, the controlled release material of the dosage form may function to provide duodenal delivery, jejunal delivery, ileal delivery, ileo-colonic delivery or colonic delivery.

In some embodiments, the controlled release material of the dosage form may function to provide delayed onset, rapid release or delayed onset, extended release, or delayed onset, pulsatile release

In some preferred dosage forms of the invention, the dosage form does not substantially disintegrate or dissolve in the oral cavity upon residence for up to about 0.5, 1, 2, 3, 4, 5, 7 or 10 minutes.

In some preferred dosage forms of the invention, the dosage form does not substantially adhere to the buccal mucosa upon contact for up to about 0.5, 1, 2, 3, 4, 5, 7 or 10 minutes.

In some preferred dosage forms of the invention, the dosage form excludes pharmaceutical excipients and ingredients which are intended to provide taste masking properties to minimize the bitter taste of buprenorphine.

In some preferred dosage forms of the invention, the dosage form upon prolonged residence in the oral cavity or prolonged oromucosal or buccal contact (e.g., for up to about 0.5, 1, 2, 3, 4, 5, 7 or 10 minutes) is not absorbed or substantially absorbed into the systemic circulation.

In some preferred dosage forms of the invention, the dosage form includes taste aversive agents (e.g., bittering agents) in sequestered or unsequestered form to deter sublingual, oromucosal or buccal use of the dosage form. In some embodiments, the taste aversive agents (bittering agent) is coated on the oral dosage form and then overcoated with material which prevents or minimizes the bitter sensation upon normal oral ingestion but which does not protect against an aversive taste upon prolonged residence in the oral cavity (e.g., upon sublingual, oromucosal or buccal use). In this manner the taste aversive agent is not sequestered in the sense that it is readily released in the GI tract upon oral ingestion, where it is devoid of taste aversive effects. A wide variety of pharmaceutical excipients known in the art may be used to provide the desired outer coating to the dosage form. In some embodiments, the taste aversive agents (e.g., bittering agent) is incorporated in the oral dosage form which prevents or minimizes the bitter sensation upon normal oral ingestion but which does not protect against an aversive taste upon prolonged residence in the oral cavity (e.g., upon sublingual, oromucosal or buccal use). In this manner the taste aversive agent may be sequestered or unsequestered. In some embodiments, the taste aversive agents (e.g., bittering agent) is incorporated into the inside walls of the capsule shell which prevents or minimizes the bitter sensation upon normal oral ingestion but which does not protect against an aversive taste upon prolonged residence in the oral cavity (e.g., upon sublingual, oromucosal or buccal use).

In some preferred dosage forms of the invention, the dosage form excludes sugar, or other natural or artificial sweeteners.

In some preferred dosage forms of the invention, the dosage form excludes pharmaceutical excipients and ingredients known in the art or intended to produce or enhance disintegration or dissolution in the oral cavity.

In some preferred dosage forms of the invention, the dosage form excludes pharmaceutical excipients and ingredients known in the art or intended to produce effervescence within the oral cavity.

In some preferred dosage forms of the invention, the dosage form excludes pharmaceutical excipients and ingredients known in the art or intended to enhance the transmucosal, lingual or sublingual absorption of buprenorphine.

In some preferred embodiments, the dosage form is non-releasable or substantially non-releasable until (i) after a particular time following oral ingestion, when the dosage form can be anticipated to have reached the duodenum, jejum, ileum, ileo-cecal junction, cecum, or colon; (ii) the dosage form has come in contact or substantial contact or sustained contact with a desired gastrointestinal pH environment (e.g., pH>3, or pH>3.5, or pH>4, or pH>4.5, or pH, >5, or pH>5.5, or pH>6, or pH>7, or pH>7.5, or pH>7.8); (iii) the dosage form has come in contact with desired microbial flora (e.g., colonic microbial flora).

In certain situations involving pharmacokinetic evaluations, it may not be possible to provide the same amount of drug by different routes of administration due to the lack of commercially available dosage strengths or because such administration would require testing outside the approved method of administration (e.g., simultaneous sublingual administration o five tablets). Under such circumstances, the term “after the same amount of an oral immediate release formulation of buprenorphine” may be waived and different amounts of drug may be evaluated, provided the data are dose normalized using pharmacokinetic approaches well known in the art.

The term “agonist” means a ligand that binds to a receptor and alters the receptor state resulting in a biological response. Conventional agonists increase receptor activity, whereas inverse agonists reduce it (See Neubig et al, IUPHAR Committee on Receptor Nomenclature and Classification, Pharmacol Rev, 2003; Howlett et al., Mol Pharmacol, 1988).

The term “opioid agonist” means a molecule that causes a specific physiologic, pathophysiologic or pharmacologic effect after binding to an opioid receptor.

An “antagonist” is a drug or ligand that reduces the action of another drug or ligand, generally an agonist. Many antagonists act at the same receptor macromolecule as the agonist. (See Neubig et al, IUPHAR Committee on Receptor Nomenclature and Classification, Pharmacol Rev, 2003; Howlett et al., Mol Pharmacol, 1988).

The term “receptor” means a molecule within a cell, on a cell surface, on a membrane, in tissue, in fluid or otherwise found in humans that serve as a recognition or binding site to cause specific physiologic, pathophysiologic or pharmacologic effects. The term “receptor” also means a cellular macromolecule, or an assembly of macromolecules, that is concerned directly and specifically in chemical signaling between and within cells. Combination of a hormone, neurotransmitter, drug, ligand, or intracellular messenger with its receptor(s) initiates a change in cell function (Neubig et al, IUPHAR Committee on Receptor Nomenclature and Classification, Pharmacol Rev, 2003).

The term “opioid receptor” includes mu (μ), delta (δ) and kappa (κ) opioid receptors, their subtypes and splice variants such as mu₁, mu₂, delta₁, delta₂, kappa₁, kappa₂ and kappa₃, etc.

Opioid antagonists are known or readily determined by individuals who practice the art. Preferably, the opioid antagonists useful for the present invention may be selected from the group consisting of naltrexone, methylnaltrexone, nalbuphine, naloxone, nalmefene, cyclazocine, cyclorphan, oxilorphan nalorphine, nalorphine dinicotinate, nalmefene, nadide and levallorphan.

In certain preferred embodiments of the present invention, the invention allows for the use of lower doses of buprenorphine by virtue of the inclusion or co-administration of an additional drug for the prevention or treatment of pain. By using lower amounts of either or both drugs, the side effects associated with treatment in humans are reduced.

The term “buprenorphine” means buprenorphine base, as well as their pharmaceutically acceptable salts, prodrugs, esters, analogs, derivatives, solvates, complexes, polymorphs, and hydrates, as racemates or an individual diastereoisomers or enantiomeric isomers thereof or mixtures thereof. In some preferred embodiments, the dosage form comprises buprenorphine base or their pharmaceutically acceptable salts, or mixtures thereof. In some even more preferred embodiments, the dosage form comprises buprenorphine base or buprenorphine HCl, or mixtures thereof.

The phrase “comprising a therapeutically effective amount of buprenorphine” means “comprising a therapeutically effective amount of buprenorphine or a pharmaceutically acceptable salt of buprenorphine, or prodrugs, esters, analogs, derivatives, solvates, complexes, polymorphs and hydrates thereof, as racemates or an individual diastereoisomers or enantiomeric isomers thereof or mixtures thereof.

When the dosage form includes a pharmaceutically acceptable salt, any salt may be use. Preferably, the salt is the hydrochloride salt of buprenorphine.

The singular forms “a”, “an” and “the” include plural referents unless the context clearly dictates otherwise. Thus, for example, reference to “a polymer” includes a single polymer as well as a mixture of two or more different polymers, reference to “a permeation enhancer” includes a single permeation enhancer as well as two or more different permeation enhancer in combination, and the like.

Some of the drugs disclosed herein may contain one or more asymmetric centers and may thus give rise to enantiomers, diastereomers, and other stereoisomeric forms. The present invention is also meant to encompass all such possible forms as well as their racemic and resolved forms and mixtures thereof. When the compounds described herein contain olefinic double bonds or other centers of geometric asymmetry, and unless specified otherwise, it is intended to include both E and Z geometric isomers. All tautomers are intended to be encompassed by the present invention as well.

As used herein, the term “stereoisomers” is a general term for all isomers of individual molecules that differ only in the orientation of their atoms is space. It includes enantiomers and isomers of compounds with more than one chiral center that are not mirror images of one another (diastereomers).

The term “chiral center” refers to a carbon atom to which four different groups are attached.

The term “enantiomer” or “enantiomeric” refers to a molecule that is nonsuperimposeable on its mirror image and hence optically active wherein the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule.

The “NNH” or “number needed to harm” is a measure that indicates how many patients would require a specific treatment to cause harm in one patient. As used herein, the “NNH or “number needed to harm” is a measure that includes: (i) how many opioid naive healthy subjects would require treatment to cause moderate or severe sedation (or drowsiness) in one subject, where moderate to severe sedation or drowsiness is defined as a VAS score of ≧50 mm on a 100 mm scale bounded on the left by “no sedation or drowsiness” and on the right by “extreme sedation or drowsiness”; (ii) how many opioid naive healthy subjects would require treatment to cause moderate or severe nausea in one subject, where moderate to severe nausea is defined as a VAS score of ≧50 mm on a 100 mm scale bounded on the left by “no nausea” and on the right by “extreme nausea”; (iii) how many opioid naive healthy subjects would require treatment to cause dizziness in one subject, where dizziness is defined as unsteadiness, imbalance, lightheadedness, spinning sensation or sensation that one is falling; (iv) how many opioid naive healthy subjects would require treatment to cause a sensation of dry mouth in one subject, where dry mouth is defined as abnormal dryness of the mouth associated with decreased secretion of saliva.

The “drug effects” questionnaire assesses the extent to which subjects currently felt a drug effect, on a scale of 1 to 5 (1=“I feel no effect from it at all”; 2=“I think I feel a mild effect, but I'm not sure”; 3=“I feel an effect, but it is not real strong”; 4=“I feel a strong effect”; 5=“I feel a very strong effect”). This questionnaire can be used to examine the overall drug effects, preferably in drug abusers and recreational drug users.

The “drug liking” questionnaire assesses the extent to which subjects currently like the effects of the drug on a 100-mm VAS, bounded on the left by “0=dislike a lot”, bounded on the right by “100=like a lot”. This questionnaire can be used to examine the overall drug liking of, preferably in drug abusers and recreational drug users.

The “take again” questionnaire assesses whether subjects would take the drug again if given the opportunity. The patient is asked “If given an opportunity, would you take this drug again? (circle one: YES or NO). This questionnaire can be used to examine the overall desirability of the drug experience, preferably in drug abusers and recreational drug users.

On the “coasting” questionnaire the patient is asked to put a mark on a horizontal line that best describes their response to the question: “Do you feel like you are coasting or spaced out? The horizontal line is a visual analog scale (VAS) bounded on the left by “not at all” and on the right by “extremely”. This questionnaire can be used to examine the degree to which subjects feel like they are coasting or spaced out, preferably in drug abusers and recreational drug users.

Three performance tasks may be employed for measuring skills related to driving.

The “critical tracking task” measures the patient's ability to control a displayed error signal in a first-order compensatory tracking task. The error is displayed as a horizontal deviation of a cursor from the midpoint on a horizontal, linear scale. Compensatory joystick movements correct the error by returning the cursor to the midpoint. The frequency at which the patient loses the control is the critical frequency. The critical tracking task measures the psychomotor control during a closed loop operation. It is a laboratory analog to on-the-road tracking performance.

The “stop signal task” measures motor impulsivity, which is defined as the inability to inhibit an activated or pre-cued response leading to errors of commission. The task requires patients to make quick key responses to visual go signals, i.e. the letters ABCD presented one at a time in the middle of the screen, and to inhibit any response when a visual stop signal, i.e. “*” in one of the four corners of the screen, is presented at predefined delays. The main dependent variable is the stop reaction time on stop signal trials that represents the estimated mean time required to inhibit a response.

The “Tower of London” (TOL) is a decision-making task that measures executive function and planning. The task consists of computer generated images of begin- and end-arrangements of three colored balls on three sticks. The subject's task is to determine as quickly as possible, whether the end-arrangement can be accomplished by “moving” the balls in two to five steps from the beginning arrangement by pushing the corresponding number coded button. The total number of correct decisions is the main performance measure.

In some embodiments, the dosage form of the invention, one or more or all of the specifications and claims applicable to the prevention and treatment of pain or addiction disorders is also applicable to the prevention or treatment of any other disease or disorder that responds to opioid agonists or to buprenorphine.

The prevention and treatment of all diseases and disorders is contemplated by the use of this invention, including without limitation, (i) pain; (ii) addiction disorders; (iii) opioid substitution and opioid maintenance therapy; (iv) restless leg syndrome; (v) cough; (vi) urinary incontinence; (vii) cough; (viii) pain associated with sickle cell disease, including vaso-occlusive crisis; (ix) peripheral and central neuropathic pain; (x) cancer pain; (xi) breakthrough pain; (xii) visceral pain; (xiii) dyspnea and respiratory distress; (xiv) infectious, immunologic, cardiovascular, pulmonary, gastrointestinal, hepatic, biliary, nutritional, metabolic, endocrine, hematologic, oncologic, musculoskeletal, rheumatic, neurologic, psychiatric, genitourinary, gynecologic, obstetric, pediatric, otolaryngogologic, ophthalmic, dermatologic, dental, oral, and genetic disorders, diseases and maladies and signs and symptoms thereof; (xv) depression, schizophrenia, influenza, common colds, anxiety, panic attacks, agoraphobia, ADHD, insomnia, sleep disorders, nasal congestion, headaches, migraine, urinary incontinence, constipation, allergies, cough, pneumonia, COPD, asthma, fluid retention, acid reflux, peptic ulcers, hypertension, cardiac arrhythmias, hypercholesterolemia, CHF, fever, diarrhea, back pain, myofascial pain, osteoarthritis, neuropathic pain, cancer pain, acute pain, diabetes, muscle spasms, and rheumatoid arthritis, and signs and symptoms thereof; and (xvi) disorders, diseases and maladies, and signs and symptoms thereof referred to in Harrison's Principles of Internal Medicine, 16th Edition, 2004, Kasper D L, Braunwald W, Fauci A, Hauser S, Longo D, and Jameson J L (eds)], which is hereby incorporated in its entirety by reference; said disorders, diseases and maladies, and signs and symptoms thereof comprising buprenorphine responsive medical conditions.

In some preferred embodiments, the oral pharmaceutical dosage forms of buprenorphine are used to treat pain, cough, dyspnea, opioid addiction disorders, restless leg syndrome, acute herpes zoster, visceral pain, breakthrough pain, opioid dependence and urinary incontinence.

As used herein, the term “pain” includes: (i) peripheral neuropathic pain, e.g., acute and chronic inflammatory demeyelinating polyradiculopathy, alcoholic polyneuropathy, chemotherapy-induced polyneuropathy, complex regional pain syndrome (CRPS) Type I and Type II, entrapment neuropathies (e.g., carpal tunnel syndrome), HIV sensory neuropathy, iatrogenic neuralgias (e.g., postthoracotomy pain, postmastectomy pain), idiopathic sensory neuropathy, painful diabetic neuropathy, phantom limb pain, postherpetic neuralgia, trigeminal neuralgia, radiculopathy (e.g., cervical thoracic, lumbosacral), sciatica, acute herpes zoster pain, temporomandibular joint disorder pain and postradiation plexopathy; and (ii) central neuropathic pain, e.g., compressive myelopathy from spinal stenosis, HIV myelopathy, multiple sclerosis pain, Parkinson's disease pain, postischemic myelopathy, post postradiation myelopathy, poststroke pain, posttraumatic spinal cord injury and syringomyelia; and (iii) cancer associated neuropathic pain, e.g., chemotherapy induced polyneuropathy, neuropathy secondary to tumor infiltration or nerve compression, phantom breast pain, postmastectomy pain, postradiation plexopathy and myelopathy; (iv) chronic pain, e.g., back pain, rheumatoid arthritis, osteoarthritis, inflammatory pain, non-inflammatory pain, myofascial pain, cancer pain, visceral pain, somatic pain, pelvic pain, musculoskeletal pain, post-traumatic pain, bone pain and idiopathic pain; (v) acute pain, e.g, acute postsurgical pain (including laparoscopic, laparatomy, gynecologic, urologic, cardiothoracic, arthroscopic, gastrointestinal, neurologic, orthopedic, oncologic, maxillofacial, ophthalmic, otolaryngologic, soft tissue, plastic, cosmetic, vascular and podiatric surgery, including abdominal surgery, abdominoplasty, adenoidectomy, amputation, angioplasty, appendectomy, arthrodesis, arthroplasty, arthroscopy, bilateral cingulotomy, biopsy, brain surgery, breast biopsy, cauterization, cesarean section, cholecystectomy, circumcision, commissurotomy, cordotomy, corneal transplantation, cricothoracotomy, discectomy, diverticulectomy, episiotomy, endarterectomy, endoscopic thoracic sympathectomy, foreskin restoration, fistulotomy, frenectomy, frontalis lift, fundectomy, gastrectomy, grafting, heart transplantation, hemicorporectomy, hemorrhoidectomy, hepatectomy, hernia repair, hypnosurgery, hysterectomy, kidney transplantation, laminectomy, laparoscopy, laparotomy, laryngectomy, lithotripsy, lobotomy, lumpectomy, lung transplantation, mammectomy, mammoplasty, mastectomy, mastoidectomy, mentoplasty, myotomy, mryingotomy, nephrectomy, nissen fundoplication, oophorectomy, orchidectomy, parathyroidectomy, penectomy, phalloplasty, pneumotomy, pneumonectomy, prostatectomy, psychosurgery, radiosurgery, ritidoplasty, rotationplasty, sigmoidostomy, sphincterotomy, splenectomy, stapedectomy, thoracotomy, thrombectomy, thymectomy, thyroidectomy, tonsillectomy, tracheotomy, tracheostomy, tubal ligation, ulnar collateral ligament reconstruction, ureterosigmoidostomy, vaginectomy, vasectomy, vulvectomy; renal colic; incisional pain; inflammatory incisional pain; nociceptive incisional pain; acute neuropathic incisional pain following surgery), renal colic, trauma, acute back pain, burn pain, burn dressing change pain, migraine pain, tension headache pain, acute musculoskeletal pain, acute exacerbation or flare of chronic back pain, acute exacerbation or flare of osteoarthritis, acute exacerbation or flare of chronic pain, breakthrough chronic non-cancer pain, breakthrough cancer pain, acute exacerbation or flare of rheumatoid arthritis, acute exacerbation or flare of myofacsial pain, acute exacerbation or flare of chronic idiopathic pain, acute exacerbation or flare of neuropathic pain, procedure related pain (e.g., arthroscopy, laparoscopy, endoscopy, intubation, bone marrow biopsy, soft tissue biopsy, catheterization), and other self-limiting pain states.

As used herein, the term “acute pain” refers to self-limiting pain that subsides over time and usually lasting less that about 30 days and more preferably lasting less than about 21 days. Acute pain does not include chronic conditions such as chronic neuropathy, chronic neuropathic pain and chronic cancer and non-cancer pain.

As used herein, “neuropathic pain” is pain initiated or caused by a primary lesion or dysfunction of the nervous system and includes (i) peripheral neuropathic pain and (ii) central neuropathic pain.

As used herein, the term “chronic pain” includes all non-neuropathic pain usually lasting more than 30 days, including inflammatory pain, non-inflammatory pain, muscle pain, joint pain, fascia pain, visceral pain, bone pain and idiopathic pain.

The term “analgesic effectiveness” is defined for purposes of the present invention as a satisfactory prevention, reduction in or elimination of pain, along with a tolerable level of side effects, as determined by the human patient.

According to the American Academy of Pain Medicine, the American Pain Society and the American Society of Addiction Medicine “addiction” and “addiction disorder” is a primary, chronic, neurobiologic disease, with genetic, psychosocial, and environmental factors influencing its development and manifestations. It is characterized by behaviors that include one or more of the following: impaired control over medication use, compulsive use, continued use despite harm, and craving (Sloan and Babul, Expert Opinion on Drug Delivery 2006; 3:489-97). The pharmaceutical composition of the present invention is in some embodiments intended to treat addiction disorder, particularly opioid addiction disorder and poly-substance abuse involving opioids. In some embodiments, the dosage form of the invention is intended to reduce or eliminate the craving or desire for opioids and the antisocial, medically harmful and potentially criminal behavior of the patient with the addiction disorder. The use of sublingual buprenorphine for the treatment of addiction disorder has been well established in the literature.

The term “therapeutic effectiveness” is defined for purposes of the present invention as a satisfactory prevention, reduction in or elimination of signs and symptoms of the medical disorder, disease or syndrome (e.g., pain, addiction disorder), along with a tolerable level of side effects, as determined by the human patient.

As used herein, the “Orange Book” as it is commonly known is the database of Approved Drug Products with Therapeutic Equivalence Evaluations maintained by or on behalf of the US Food and Drug Administration, (http://www.fda.gov/cder/ob/default.htm, accessed Feb. 15, 2008), the content of which is hereby incorporated by reference.

“Drug”, “drug substance”, “substance”, “therapeutic”, “therapeutic agent”, “pharmacological agent”, “pharmaceutical agent”, “active agent”, “active ingredient”, “agent” “active pharmaceutical ingredient” or “API” are used interchangeably and are intended to have their broadest interpretation as to any therapeutically active substance which is delivered to a living organism to furnish pharmacological activity or other direct effect in the diagnosis, cure, mitigation, treatment, or prevention of a disease, or to affect the structure or any function of the human body. In general, this includes therapeutic agents in all of the major therapeutic areas.

The term “subject” for purposes of treatment is used interchangeably with “patient”, “male”, “female”, and includes any human subject or mammal. It will be obvious to any practitioner of the art that some of the embodiments, specifications and claims of the invention are not applicable to non-human mammals or need to be modified. For example, the dose of invention may vary depending on the species, its weight and metabolism. Similarly, certain behaviors (e.g., drug abuse, drug diversion) and pharmacodynamic effects (e.g., assessment of mood altering effects or cognitive impairment) are not usually directly assessed in or applicable to many non-human species. Preferred non-human mammals are mammals whose medical care is provided by veterinarians, veterinary technicians, paraveterenarians, animal agriculture industry personnel, and game and wildlife personnel. Preferred non-human mammals include domesticated farm animals, pets, livestock and wild game.

“Pharmaceutically or therapeutically acceptable excipient or carrier” or “excipient” refers to a substance which does not interfere with the effectiveness or the biological activity of the active ingredients and which is not toxic to the subject. In some embodiments of the present invention, pharmaceutically or therapeutically acceptable excipients or carriers may play a role in imparting or optimizing the rate and extent of absorption or buprenorphine or additional drugs in the pharmaceutical composition. In some embodiments of the present invention, pharmaceutically or therapeutically acceptable excipients or carriers may play a role in stabilizing the buprenorphine or additional drugs in the pharmaceutical composition. Excipients are widely known in the art (see, for example, FDA EAFUS database (http://vm.cfsan.fda.gov/˜dms/eafus.html); FDA Food Additives Status List (http://www.cfsan.fda.gov/˜dms/opa-appa.html); FDA GRAS list and database; FDA Color Additive Status List (http://www.cfsan.fda.gov/˜dms/opa-appc.html); FDA Inactive Ingredients Database (http://www.accessdata.fda.gov/scripts/cder/iig/index.cfm); Rowe, Sheskey and Owen, Handbook of Pharmaceutical Excipients, APhA Publications; 5th edition (2006); Rowe, Sheskey and Quinn, Handbook of Pharmaceutical Excipients, 6 edition, Pharmaceutical Press; APhA Publications; 2009; Goodman & Gilman's The Pharmacological Basis of Therapeutics (Brunton, Lazo and Parker, eds, 11 th ed., McGraw Hill (2005); Remington: The Science and Practice of Pharmacy, 21st ed, Lippincott Williams & Wilkins (2005); Martindale: The Complete Drug Reference, 35th Edition, Pharmaceutical Press (2007); United States Pharmacopeia—National Formulary (USP-NF), (USP-NF 25, 2007), the International Programme on Chemical Safety (http://www.inchem.org/) and Health Canada's List of Acceptable Non-medicinal Ingredients (http://www.hc-sc.gc.ca/dhp-mps/prodnatur/legislation/docs/nmi-imn_list1_e.html), all hereby incorporated by reference in their entirety).

In certain preferred embodiments of the present invention, an effective amount of buprenorphine in immediate release form is included in the controlled release unit dose buprenorphine formulation to be administered. The immediate release form of the buprenorphine is preferably included in an amount which is effective to shorten the time to Cₘₐₓ of the buprenorphine in the blood (e.g., plasma). One skilled in the art would recognize various means of incorporating the immediate release buprenorphine into the unit dose. By including such an effective amount of immediate release buprenorphine in the unit dose, patients may experience superior relief of pain and neuropathy symptoms.

In certain preferred embodiments of the present invention, the dosage form may include, in addition to the buprenorphine, substances, process or technologies that impart abuse deterrent, abuse resistant or tamper resistant properties to the dosage form, including aversive agents; said dosage form reducing or preventing opioid abuse, drug abuse, drug misuse, recreational drug use, drug diversion and toxicity from intentional or accidental overdose.

As used herein, the term “aversive”, “aversive agents”, “aversion producing agents” and “aversive compounds” means to compounds contained within the dosage form that produce an aversive, undesirable, repugnant, distasteful, unpleasant, unacceptable physiologic effect, unacceptable psychic effect, or that pharmacologically block or reduce physiologic effects sought by recreational drug users, addicts and drug abusers, including one or more of the following effects: mood alterations; euphoria, pleasure; a feeling of high; a feeling of drug liking; anxiolysis; sedation; calmness; a state of relaxation; psychotomimesis; hallucinations; alterations in perception, cognition and mental focus; drowsiness; and psychological reinforcement.

In certain preferred embodiments of the present invention, the dosage form may include, in addition to buprenorphine or a pharmaceutically acceptable salt thereof, abuse deterrent or abuse resistant substances, process or technologies known in the art, including one or more aversive agents. All kinds of aversive agents are contemplated, including, without limitation, antagonists of abusable drugs, laxatives, cutaneous vasodilators, headache producing agents, emetics, emetogenic compound, nausea producing compounds, bittering agents, drugs that cause burning on irritation when in contact with tissue or mucous membranes (e.g., naso-mucosal irritants, oro-mucosal irritants, respiratory irritants), tissue irritants, gastrointestinal irritants, drugs that precipitate withdrawal effects, tissue dyes, lakes and colorants, beverage dyes, lakes and colorants, non-tissue staining beverage dyes, lakes and colorants (i.e., that do not stain or discolor the skin upon ingestion), fecal discolorants, urine discolorants, malodorous agents, opioid antagonists, benzodiazepine antagonists (e.g., flumazenil), cannabinoid antagonists and pharmacologic antagonists to co-abused drugs not contained in the dosage form. Such aversive agents may be in the dosage form in a releasable, partially releasable or a non-releasable form (i.e., sequestered), the latter being released on tampering the dosage form (e.g., mechanical, thermal, chemical, solvent tampering, ingestion in ways not recommended, and the like). Further, in some embodiments, such aversive agents may be in the dosage form in an amount that does not produce an aversive effect or aversion in any, many or substantially all patients when taken in accordance with the prescribing information or the manufacturer's instructions (for example, in small quantities), but which produce an aversive effect when taken in excess (e.g., higher dose or more frequently). In other embodiments, said aversive agent pharmacologically blocks the effects of the buprenorphine and/or the effects of a co-abused drug, said co-abused drug in the same dosage form or in a different dosage form or not an approved or conventional pharmaceutical product.

The term “tampering” or “tamper” means any manipulation by mechanical, thermal, chemical and/or pharmacologic means which changes the physical or chemical properties of the dosage form, e.g., to liberate the abusable drugs for immediate release if it is in sustained release form, or to make the abusable drugs available for inappropriate use such as administration by an alternate route, e.g., parenterally. The tampering can be, e.g., by means of crushing, shearing, grinding, mechanical extraction, solvent extraction, solvent immersion, combustion, heating or any combination thereof.

The term “abuse”, “drug abuse”, “opioid abuse”, “recreational drug use” and “drug misuse” in the context of the present invention means, use: (i) in quantities or by methods and routes of administration that do not conform to standard medical practice; (ii) outside the scope of specific instructions for use provided by a qualified medical professional; (iii) outside the supervision of a qualified medical professional; (iv) outside the approved instructions on proper use provided by the drug's legal manufacturer; (v) which is not in specifically approved dosage forms for medical use as pharmaceutical agents; (vi) where there is an intense desire for and efforts to procure same; (vii) compulsive use; (viii) through acquisition by manipulation of the medical system, including falsification of medical history, symptom intensity, disease severity, patient identity, doctor shopping, prescription forgeries; (ix) where there is impaired control over use; (x) despite harm; (xi) by procurement from non-medical sources; (xii) by others through sale or diversion by the individual into the non-medical supply chain; (xiii) for medically unapproved or mood altering purposes.

The term “mood altering” is defined for purposes of the present invention to mean that the “high”, “liking”, pleasurable, euphoric, alerting, calming, anxiolytic, auditory and visual perception altering, relaxing, psychotomimetic, rewarding and reinforcing, of the abusable drug.

The term “abuse resistant”, “abuse deterrent”, “tamper resistant”, “deter abuse” and “resist abuse” (as well of the words “resist” or “deter” when applied to abusable drugs of the invention) are used interchangeably in the context of the present invention and include pharmaceutical compositions and methods that resist, deter, discourage, diminish, delay and/or frustrate: (i) the physical, chemical, thermal or pharmacologic manipulation or tampering of the dosage form (e.g., crushing, shearing, grinding, chewing, dissolving, melting, needle aspiration, inhalation, insufflation, extraction by mechanical, thermal and chemical means, and/or filtration); (ii) use or misuse of the dosage form outside the scope of specific instructions for use provided by a qualified medical professional; (iii) use outside the supervision of a qualified medical professional; (iv) use outside the approved instructions on proper use provided by the drug's legal manufacturer (e.g., intravenous use, intranasal use, inhalational use and oral ingestion to provide high peak concentrations, use in excess quantities, etc.); (v) the conversion of an extended release dosage form of the invention into a more immediate release form; (vi) the intentional and iatrogenic increase in physical and psychic effects sought by recreational drug users, addicts, and patients with pain who have an addiction disorder; (vii) attempts to procure the dosage form by manipulation of the medical system and from non-medical sources; (viii) the sale or diversion of the dosage form into the non-medical supply chain and for medically unapproved or unintended mood altering purposes; (ix) the intentional, unintentional or accidental attempts at otherwise changing the physical, pharmaceutical, pharmacological and/or medical properties of the dosage form from what was intended by the manufacturer; (x) the psychic, pleasurable, reinforcing or rewarding effects of the dosage form when used as directed or when used outside the approved instructions on proper use provided by the drug's legal manufacturer.

All kinds of abuse deterrent agents, excipients, dosage forms and technologies are contemplated, including, without limitation, excipients that deter or resist extraction of drug with the application of mechanical, chemical, or thermal energy, use of solvents, use of sequestered or unsequestered (releasable) antagonists to the drug or to a co-abused drug, use of sequestered or unsequestered (releasable) aversive agents, and use covalently bound moieties that modulate release of the buprenorphine in vitro, in the GI tract and in the liver.

In some embodiments, one or more aversive agents may be added to the formulation in an amount of less than about 80% by weight, preferably less than about 60% by weight, more preferably less than about 40% by weight of the dosage form, even more preferably less than about 20% by weight of the dosage form, and most preferably less than about 10 by weight of the dosage form (e.g., 0.000000000000001% to 1%, or 0.000000001% to 3%, or 0.0001% to 10%, or 0.001% to 5%, or 1% to 10%, or 0.001% to 2%, or 1% or 10%, or 2% to 7%) depending on the particular aversive agent used.

In some embodiments, the aversive agent in the dosage form may be about 0.00000000001 mg to about 2000 mg, or about 0.0000001 mg to about 1500 mg, or about 0.000001 mg to about 1000 mg, or about 0.0001 mg to about 1000 mg, or about 0.001 mg to about 1000 mg, or about 0.01 mg to about 1000 mg, or about 0.1 mg to about 1500 mg, or 1 mg to about 800 mg, or about 1 mg to about 500 mg, or about 1 mg to about 300 mg, or about 1 mg to about 150 mg, or about 5 mg to about 400 mg, or about 5 mg to about 200 mg, or about 0.00000000001 mg to about 200 mg, or about 0.00000000001 mg to about 100 mg, or about 0.00000000001 mg to about 50 mg, or about 0.0000001 mg to about 200 mg, or about 0.0000001 mg to about 100 mg, or about 0.00001 mg to about 400 mg, or about 0.0001 mg to about 300 mg.

As described above, the present invention can include one or more aversive agents, selected from the group including, without limitation antagonists of abusable drugs, laxatives, cutaneous vasodilators headache producing agents, emetics, emetogenic compound, nausea producing compounds, bittering agents, drugs that cause burning on irritation when in contact with tissue or mucous membranes (e.g., naso-mucosal irritants, oro-mucosal irritants, respiratory irritants), tissue irritants, gastrointestinal irritants, drugs that precipitate withdrawal effects, tissue dyes, lakes and colorants, beverage dyes, lakes and colorants, non-tissue staining beverage dyes, lakes and colorants, fecal discolorants, urine discolorants, malodorous agents, opioid antagonists, benzodiazepine antagonists, cannabinoid antagonists, and pharmacologic antagonists to co-abused drugs not contained in the dosage form. Preferably, the aversive agent is a pharmaceutically acceptable agent that produces an aversive effect only when the dosage form of the invention containing the aversive agent is abused, for example, when taken in excess of medically approved doses, taken in excess of approved doses in the manufacturer's prescribing information, taken after tampering of the dosage form (e.g., mechanical, thermal, chemical, solvent tampering), ingestion in ways not medically recommended, administration by routes not approved for the dosage form (e.g., intranasal, inhalation, intravenous) or in a manner inconsistent with the manufacturer's prescribing information.

In some embodiments, the amount of aversive agent in the dosage form of the present invention can be a fixed ratio in relation to the amount of abusable drug in the dosage form. By appropriately selecting the quantity of the aversive agent in the dosage form, aversive effects can be avoided under conditions of proper medical use (e.g., manufacturers prescribing directions). However, under some conditions of abuse, for example excessive intake of the dosage form of the invention, the quantity of aversive agent consumed will exceed the “no effect” or “minimum effect” threshold, thereby producing one or more aversive effects, for example, e.g., nausea, emesis, diarrhea, laxation, cutaneous vasodilation, headache, bitter taste, naso-mucosal irritation, oro-mucosal irritation, precipitation of abstinence from the abusable drug of the dosage form, precipitation of abstinence from a co-abused drug which is not part of the dosage form, reduction of the pleasurable, mood altering, rewarding, reinforcing, stimulant, depressant or other psychic and physiologic effects of the abusable drug or a co-abused drug, etc.).

In some embodiments, the “no effect” or “minimum effect” threshold amount of aversive agent can be exceeded when the dosage form of the invention is taken in excess of the manufacturer's recommendation by a factor of about 1.5, or about 2, or about 2.5, or about 3, or about 4, or about 5, or about 6, or about 7, or about 8, or about 10, or more than 10. In some embodiments, the production of an aversive effect can reduce or stop further abuse of the dosage form, thereby reducing the harm or toxicity of the drug in the subject who is tampering, misusing or abusing the dosage form, e.g., addicts, drug abusers and recreational drug users.

Various bittering agents can be employed including, for example and without limitation, T2R or TAS2R receptor agonists, phenylthiourea (phenylthiocarbamide), natural, artificial and synthetic flavor oils and flavoring aromatics and/or oils, oleoresins and extracts derived from plants, leaves, flowers, fruits, and so forth, and combinations thereof. Nonlimiting representative flavor oils include spearmint oil, peppermint oil, eucalyptus oil, oil of nutmeg, allspice, mace, oil of bitter almonds, menthol and the like. Also useful bittering agents are artificial, natural and synthetic fruit flavors such as citrus oils including lemon, orange, lime, grapefruit, and fruit essences and so forth. Additional bittering agents include sucrose derivatives (e.g., sucrose octaacetate), chlorosucrose derivatives, quinine and quinine salts, quinidine and quinidine salts and the like. The preferred bittering agent for use in the present invention is denatonium, denatonium benzoate and denatonium saccharide. A dosage form including a bittering agent preferably discourages improper usage of the tampered dosage form by imparting a disagreeable taste to the tampered dosage form.

In some embodiments, the aversive agent in the dosage form may be denatonium, denatonium saccharide or denatonium benzoate, in a quantity expressed as mg of denatonium, of about 0.00000001 mg to about 100 mg, or about 0.000001 mg to about 100 mg, or about 0.0001 mg to about 100 mg, or about 0.0001 mg to about 20 mg, or about 0.0001 mg to about 10 mg, or about 0.0001 mg to about 5 mg, or about 0.0001 mg to about 2 mg, or about 0.0001 mg to about 1 mg, about 0.0001 mg to about 50 mg, or about 0.00000001 mg to about 50 mg, or about 0.00000001 mg to about 20 mg, or about 0.01 mg to about 20 mg, or about 0.01 mg to about 10 mg, or about 0.01 mg to about 5 mg, or about 0.01 mg to about 2 mg, or about 0.01 mg to about 1 mg, or about 0.01 mg to about 1 mg, or about 0.01 mg to about 0.5 mg, or about 0.1 mg to about 20 mg, or about 0.1 mg to about 10 mg, or about 0.1 mg to about 7 mg, or about 0.1 mg to about 5 mg, or about 0.1 mg to about 3 mg, or about 0.1 mg to about 2 mg.

In some embodiments, the aversive agent in the dosage form may be quinine or a pharmaceutically acceptable salt of quinine, in a quantity expressed as mg of quinine, of about 0.00001 mg to about 300 mg, or about 0.00001 mg to about 200 mg, or about 0.00001 mg to about 100 mg, or about 0.00001 mg to about 75 mg, or about 0.00001 mg to about 50 mg, or about 0.00001 mg to about 25 mg, or about 0.00001 mg to about 20 mg, or about 0.00001 mg to about 10 mg, or about 0.00001 mg to about 5 mg, or about 0.00001 mg to about 2.5 mg, or about 0.00001 mg to about 1 mg, or about 0.001 mg to about 300 mg, or about 0.001 mg to about 200 mg, or about 0.001 mg to about 100 mg, or about 0.001 mg to about 75 mg, or about 0.001 mg to about 50 mg, or about 0.001 mg to about 25 mg, or about 0.001 mg to about 20 mg, or about 0.001 mg to about 10 mg, or about 0.001 mg to about 5 mg, or about 0.001 mg to about 2.5 mg, or about 0.001 mg to about 1 mg, or about 1 mg to about 300 mg, or about 1 mg to about 200 mg, or about 1 mg to about 100 mg, or about 1 mg to about 75 mg, or about 1 mg to about 50 mg, or about 1 mg to about 25 mg, or about 1 mg to about 20 mg, or about 1 mg to about 10 mg, or about 1 mg to about 5 mg, or about 1 mg to about 2.5 mg.

Various emetic agents can be employed including, for example and without limitation, zinc and pharmaceutically acceptable salts thereof (e.g., zinc oxide, zinc gluconate, zinc acetate, zinc sulfate, zinc carbonate), dopamine agonists, apomorphine, ipecac, ipecacuanha, emetine, emetine (methylcephaeline), cephaeline, psychotrine, O-methylpsychotrine, ammonium chloride, potassium chloride, magnesium sulfate, ferrous gluconate, ferrous sulfate, aloin, algarot or antimonious oxychloride, antimony trichloride, folate, folic acid, niacin (niacin) and nicotinamide.

In some embodiments, the aversive agent in the dosage form may be zinc in the form of elemental zinc or a pharmaceutically acceptable salt of zinc, in a quantity expressed as mg of elemental zinc, of about 1 mg to about 400 mg, or about 1 mg to about 300 mg, or about 1 mg to about 200 mg, or about 1 mg to about 150 mg, or about 1 mg to about 100 mg, or about 1 mg to about 90 mg, or about 1 mg to about 80 mg, or about 1 mg to about 70 mg, or about 1 mg to about 60 mg, or about 1 mg to about 50 mg, or about 1 mg to about 45 mg, or about 1 mg to about 40 mg, or about 1 mg to about 40 mg, or about 1 mg to about 35 mg, or about 1 mg to about 30 mg, or about 1 mg to about 25 mg, or about 1 mg to about 20 mg, or about 1 mg to about 10 mg, or about 1 mg to about 5 mg, or about 5 mg to about 400 mg, or about 5 mg to about 300 mg, or about 5 mg to about 200 mg, or about 5 mg to about 150 mg, or about 5 mg to about 100 mg, or about 10 mg to about 150 mg, or about 10 mg to about 100 mg, or about 5 mg to about 80 mg, or about 5 mg to about 60 mg, or about 5 mg to about 50 mg, or about 5 mg to about 45 mg, or about 5 mg to about 40 mg, or about 5 mg to about 40 mg, or about 5 mg to about 35 mg, or about 5 mg to about 30 mg, or about 5 mg to about 25 mg, or about 5 mg to about 20 mg, or about 5 mg to about 10 mg, or about 10 mg to about 90 mg, or about 10 mg to about 80 mg, or about 10 mg to about 60 mg, or about 10 mg to about 50 mg, or about 10 mg to about 45 mg, or about 10 mg to about 40 mg, or about 10 mg to about 40 mg, or about 10 mg to about 35 mg, or about 10 mg to about 30 mg, or about 10 mg to about 25 mg, or about 10 mg to about 20 mg, or about 20 mg to about 100 mg, or about 20 mg to about 90 mg, or about 20 mg to about 80 mg, or about 20 mg to about 60 mg, or about 20 mg to about 50 mg, or about 20 mg to about 45 mg, or about 20 mg to about 40 mg, or about 20 mg to about 35 mg, or about 20 mg to about 30 mg, or about 15 mg to about 50 mg, or about 15 mg to about 40 mg, or about 15 mg to about 35 mg, or a quantity sufficient to be produce an aversive effect vasodilation when abused but not under conditions of medically appropriate use.

Various irritants can be employed including, for example and without limitation transient receptor potential vanilloid 1 (TRPV1 or VR1) agonists (including resiniferanoids, capsaicinoids, phorboid vanilloids, and terpenoid 1,4-unsaturated dialdehydes, capsaicin, capsaicin analogs and derivatives, resiniferatoxin, olvanil, piperine, zingerone, anandamide, 12- and 15-(S)-hydroperoxy-eicosatetraenoic acids, 5 and 15-(S)-hydroxyeicosatetraenoic acids, phorbol 12-phenylacetate 13-acetate 20-homovanillate, 2 phorbol 12,13-didecanoate 20-homovanillate, leukotriene B(4), tinyatoxin, heptanoylisobutylamide, N-(3-acyloxy-2-benzylpropyl)-N′-dihydroxytetrahydrobenzazepine, tetrahydroisoquinoline thiourea analogs, heptanoyl guaiacylamide, other isobutylamides or guaiacylamides, dihydrocapsaicin, homovanillyl octylester and nonanoyl vanillylamide), acids such as acids with one or more carboxyl moieties (e.g., formic acid, acetic acid, propionic acidy, butyric acid, valeric acid, caproic acid, caprillic acid, capric acid, oxalic acid, malonic acid, succicnic acid, glutaric acid, adipic acid, maleic acid, fumaric acid, and citric acid), sodium lauryl sulfate, poloxamer, sorbitan monoesters, glyceryl monooleates, niacin, mustard, allyl isothiocyaanate and p-hydroxybenzyl isothiocyanate, acetylsalicylic acid.

Various cutaneous vasodilators can be employed including, for example and without limitation, niacin acid, nicotinuric acid, beta-hydroxybutyrate and nicotinic receptor (e.g., HM74A or GPR109A) agonists.

In some embodiments, the aversive agent in the dosage form may be niacin, in a quantity of about 1 mg to about 400 mg, or about 1 mg to about 300 mg, or about 1 mg to about 200 mg, or about 1 mg to about 150 mg, or about 1 mg to about 100 mg, or about 1 mg to about 90 mg, or about 1 mg to about 80 mg, or about 1 mg to about 70 mg, or about 1 mg to about 60 mg, or about 1 mg to about 50 mg, or about 1 mg to about 45 mg, or about 1 mg to about 40 mg, or about 1 mg to about 40 mg, or about 1 mg to about 35 mg, or about 1 mg to about 30 mg, or about 1 mg to about 25 mg, or about 1 mg to about 20 mg, or about 1 mg to about 10 mg, or about 1 mg to about 5 mg, or about 5 mg to about 400 mg, or about 5 mg to about 300 mg, or about 5 mg to about 200 mg, or about 5 mg to about 150 mg, or about 5 mg to about 100 mg, or about 10 mg to about 150 mg, or about 10 mg to about 100 mg, or about 5 mg to about 80 mg, or about 5 mg to about 60 mg, or about 5 mg to about 50 mg, or about 5 mg to about 45 mg, or about 5 mg to about 40 mg, or about 5 mg to about 40 mg, or about 5 mg to about 35 mg, or about 5 mg to about 30 mg, or about 5 mg to about 25 mg, or about 5 mg to about 20 mg, or about 5 mg to about 10 mg, or about 10 mg to about 90 mg, or about 10 mg to about 80 mg, or about 10 mg to about 60 mg, or about 10 mg to about 50 mg, or about 10 mg to about 45 mg, or about 10 mg to about 40 mg, or about 10 mg to about 40 mg, or about 10 mg to about 35 mg, or about 10 mg to about 30 mg, or about 10 mg to about 25 mg, or about 10 mg to about 20 mg, or about 20 mg to about 100 mg, or about 20 mg to about 90 mg, or about 20 mg to about 80 mg, or about 20 mg to about 60 mg, or about 20 mg to about 50 mg, or about 20 mg to about 45 mg, or about 20 mg to about 40 mg, or about 20 mg to about 35 mg, or about 20 mg to about 30 mg, or about 15 mg to about 50 mg, or about 15 mg to about 40 mg, or about 15 mg to about 35 mg, or a quantity sufficient to be produce an aversive effect when abused but not under conditions of medically appropriate use.

Various tissue dyes, lakes and colorants, beverage dyes, lakes and colorants, non-tissue staining beverage dyes, lakes and colorants, fecal discolorants, urine discolorants can be employed as aversive agents including, for example and without limitation, Curcumin, Riboflavin, Tartrazine, Quinoline yellow, Sunset yellow FCF, Carmine, Carmoisine, Amaranth, Ponceau 4R, Erythrosine, Allura red AC, Patent blue V, Indigo carmine, Brilliant blue FCF, Chlorophylls, Copper complexes of chlorophylls and chlorophyllins, Green S, Caramel, Brilliant black BN, Vegetable carbon, Carotenoids, Alpha-, beta-, gamma-carotene, Capsanthin, Capsorubin, Lycopene, Beta-apo-8′ carotenal, Ethyl ester of beta-apo-8′ carotenoic acid, Xanthophylls, Lutein, Canthaxanthin, Beetroot red, Anthocyanins, Cyanidin, Delphidin, Malvidin, Pelargonidin, Peonidin, Petunidin, Calcium carbonate, Titanium dioxide, Iron oxides and hydroxides, Aluminum, Brilliant blue FCF, Indigotine, Alphazurine FG, Indanthrene blue, Fast green FCF, Alizarin cyanine green F, Quinizarine green SS, Pyranine concentrated, Orange II, Dibromofluorescein, Diiodofluorescein, Erythrosine yellowish Na, Erythrosine, Ponceau SX, Lithol rubin B, Lithol rubin B Ca, Toney red, Tetrabromofluorescein, Eosine, Tetrachlorotetrabromofluorescein, Phloxine B, Helindone pink CN, Brilliant lake red R, Acid fuchsine, Lake bordeaux B, Flaming red, Alba red, Allura red AC, Allura Red AC, Alizurol purple SS, Tartrazine, Sunset yellow, FCF, Fluorescein, Naphthol yellow S, Uranine, Quinoline yellow WS, Quinoline yellow SS, Brilliant blue FCF, Indigotine, Alphazurine FG, Alizurol purple SS, Sunset yellow FCF, Alumina, Aluminum powder, Annatto extract, Beta-carotene, Bismuth oxychloride, Bronze powder, Calcium carbonate, Canthaxanthin, Caramel, Chromium-cobalt-aluminum oxide, Chromium hydroxide green, Chromium oxide green, Cochineal extract, carmine, Copper powder, Dihydroxyacetone, Ferric ammonium citrate, Ferric ammonium ferrocyanide, Ferric ferrocyanide, Guanine, Iron oxides synthetic, Logwood extract, Mica, Potassium sodium copper chlorophyllin, Pyrogallol, Pyrophyllite, Talc, Titanium dioxide, Zinc oxide, FD&C blue #1, FD&C blue #2, D&C blue #4, D&C blue #9, FD&C green #3, D&C green #5, D&C green #6, D&C green #8, D&C orange #4, D&C orange #5, D&C orange #10, D&C orange #11, FD&C red #3, FD&C red #4, D&C red #6, D&C red #7, D&C red #17, D&C red #21, D&C red #22, D&C red #27, D&C red #28, D&C red #30, D&C red #31, D&C red #33, D&C red #34, D&C red #36, D&C red #39, FD&C red #40, FD&C red #40 lake, D&C violet #2, FD&C yellow #5, FD&C yellow #6, D&C yellow #7, Ext. D&C yellow #7, D&C yellow #8, D&C yellow #10, D&C yellow #11, FD&C lakes, D&C lakes, Ext. D&C lakes, FD&C blue #1 lake, FD&C blue #2 lake, D&C blue #4 lake, FD&C green #3 lake, D&C green #5 lake, D&C green #6 lake, D&C orange #4 lake, D&C orange #5 lake, D&C orange #10 lake, D&C orange #11 lake, FD&C red #4 lake, D&C red #6 lake, D&C red #7 lake, D&C red #17 lake, D&C red #21 lake, D&C red #22 lake, D&C red #27 lake, D&C red #28 lake, D&C red #30 lake, D&C red #31 lake, D&C red #33 lake, D&C red #34 lake, D&C red #36 lake, D&C violet #2 lake, FD&C yellow #5 lake, FD&C yellow #6 lake, D&C yellow #7 lake, Ext. D&C yellow #7 lake, D&C yellow #8 lake, D&C yellow #10 lake, Turmeric, Lactoflavin, Cochineal, carminic acid, Indigotine, Magnesium chlorophyll, Brilliant green BS, Black PN, Carbo medicinalis vegetabilis, Paprika oleoresin, Paprika oleoresin, Betanin, Beta-carotene, indigo carmine, iron oxides, sunset yellow FCF, titanium dioxide, E100, E101, E102, E104, E110, E120, E122, E123, E124, E127, E129, E131, E132, E133, E140, E141, E142, E150, E151, E153, E160, E161, E162, E163, E170, E171, E172, E173 and phenazopyridine.

As used herein, “dyes”, “lakes”, “colorants” and “discolorants” are used interchangeably and refer to one or more pharmaceutically acceptable dyes, lakes or colorants which may be: (i) tissue staining; (ii) non-tissue staining; (iii) beverage staining; (iv) urine discolorant; and/or (v) fecal discolorant.

Various laxatives can be employed as aversive agents including, for example and without limitation, Bis(p-hydroxyphenyl)pyridyl-2-methane, Bisacodyl, bisoxatin, anthraquinone, anthraquinone analogs and derivatives (e.g., buckthorn, casanthranol, cascara, hydroxyanthracene, glucofrangulin), dantron, danthron, docusate (e.g., docusate sodium, docusate calcium, docusate potassium), gastrointestinal chloride channel activators (e.g., chloride channel subtype 2 activators), lubiprostone, magenesium salts (e.g., magnesium citrate, magnesium hydroxide, magnesium oxide), mannitol, oxyphenisatine, polyethylene glycol, poly(ethylene oxide) [PEO-1500], sodium phosphate, phenolphthalein, senna, senna constituents and derivatives (e.g., sennoside A, sennoside B) and sodium picosulfate.

In some embodiments, the aversive agent in the dosage form may be a laxative in the amount of about 0.001 mg to about 300 mg, or about 0.001 mg to about 200 mg, or about 0.001 mg to about 100 mg, or about 0.001 mg to about 75 mg, or about 0.001 mg to about 50 mg, or about 0.001 mg to about 25 mg, or about 0.001 mg to about 20 mg, or about 0.001 mg to about 10 mg, or about 0.001 mg to about 5 mg, or about 0.001 mg to about 2.5 mg, or about 0.001 mg to about 1 mg, or about 1 mg to about 300 mg, or about 1 mg to about 200 mg, or about 1 mg to about 100 mg, or about 1 mg to about 75 mg, or about 1 mg to about 50 mg, or about 1 mg to about 25 mg, or about 1 mg to about 20 mg, or about 1 mg to about 10 mg, or about 1 mg to about 5 mg, or about 1 mg to about 2.5 mg.

In some embodiments, the aversive agent in the dosage form may be an opioid antagonist. Opioid antagonists are well known in the art and include naltrexone, methylnaltrexone, naloxone, nalmefene, cyclazocine, cyclorphan, oxilorphan nalorphine and levallorphan or pharmaceutically acceptable salt thereof or mixture thereof. In a preferred embodiment, said antagonist is naltrexone or naloxone. In a most preferred embodiment, said antagonist is naloxone. In some embodiments, the aversive agent in the dosage form may be an opioid antagonist in the amount of about 0.00001 mg to about 800 mg, or about 0.001 mg to about 400 mg, or about 0.01 mg to about 200 mg, or about 0.2 mg to about 100 mg, or about 0.2 mg to about 50 mg, or 0.2 to 8 mg.

In some embodiments, the dosage form comprises buprenorphine, optionally material to render said dosage form controlled release and one or more opioid antagonists, preferably selected from the group comprising naloxone, naltrexone and nalmefene; said opioid antagonist having an in vitro release rate provided herein. In some embodiments, the buprenorphine and the opioid antagonist share the same in vitro release rate (dissolution rate) specifications. In other embodiments, the buprenorphine and the opioid antagonist have different in vitro release rate (dissolution rate) specifications referred to herein. In yet other embodiments, the in vitro release rate (dissolution rate) specifications referred to herein are applicable only to the buprenorphine.

In some embodiments, the dosage form comprises buprenorphine, optionally material to render said dosage form controlled release and one or more aversive agents; said aversive agent having an in vitro release rate provided herein. In some embodiments, buprenorphine and the aversive agent share the same in vitro release rate (dissolution rate) specifications. In other embodiments, buprenorphine and the aversive agent have different in vitro release rate (dissolution rate) specifications referred to herein. In yet other embodiments, the in vitro release rate (dissolution rate) specifications referred to herein are applicable only to the buprenorphine.

Aversive agents may include compounds found on the FDA EAFUS database (http://vm.cfsan.fda.gov/˜dms/eafus.html); FDA Food Additives Status List (http://www.cfsan.fda.gov/˜dms/opa-appa.html); FDA GRAS list and database; FDA Color Additive Status List (http://www.cfsan.fda.gov/˜dms/opa-appc.html); FDA Inactive Ingredients Database (http://www.accessdata.fda.gov/scripts/cder/iig/index.cfm); Rowe, Sheskey and Owen, Handbook of Pharmaceutical Excipients, APhA Publications; 5th edition (2006); Rowe, Sheskey and Quinn, Handbook of Pharmaceutical Excipients, 6 edition, Pharmaceutical Press; APhA Publications; 2009; Goodman & Gilman's The Pharmacological Basis of Therapeutics (Brunton, Lazo and Parker, eds, 11 th ed., McGraw Hill (2005); Remington: The Science and Practice of Pharmacy, 21st ed, Lippincott Williams & Wilkins (2005); Martindale: The Complete Drug Reference, 35th Edition, Pharmaceutical Press (2007); United States Pharmacopeia—National Formulary (USP-NF), (USP 30-NF 25, 2007), the International Programme on Chemical Safety (http://www.inchem.org/) and Health Canada's List of Acceptable Non-medicinal Ingredients (http://www.hc-sc.gc.ca/dhp-mps/prodnatur/legislation/docs/nmi-imn_list1_e.html), all hereby incorporated by reference in their entirety.

It should be noted that the above mentioned aversive agents may, in some embodiments be used in the dosage form of the invention for purposes other than as aversive agents, or for both aversive and non-aversive purposes. Such non-aversive uses can include, without limitation, pharmaceutical purposes and pharmacologic purposes. For example, in some embodiments, the laxative agent may be used to counteract the constipating effects of the abusable dosage form of the invention. In some embodiments, zinc and pharmaceutically acceptable salts of zinc and niacin may be used for pharmaceutical purposes (e.g., pharmaceutical optimization, drug release and drug stability).

In some embodiments, an aversive agent incorporated into the oral dosage form shares one, or more, or all the dissolution rate specifications, GI delivery and release specifications and pharmacokinetic parameter specifications (limited to Tₘₐₓ, HVD and W₅₀) as the oral buprenorphine in the dosage form.

In some embodiments, an aversive agent incorporated into the oral dosage form has different dissolution rate specifications, GI delivery and release specifications and pharmacokinetic parameter specifications from the oral buprenorphine in the dosage form, one or more or all said different specifications contained herein.

In some embodiments, an aversive agent incorporated into the oral dosage form has different dissolution rate specifications, GI delivery and release specifications and pharmacokinetic parameter specifications from the oral buprenorphine in the dosage form.

In one embodiment of the invention, the dosage form includes both an immediate release and extended release component.

In one embodiment of the invention, the dosage form includes a capsule within a capsule, each capsule containing a different drug or the same drug intended for treating the same or a different malady. In some preferred embodiments, the outer capsule may be an enteric coated capsule or a capsule containing an immediate release formulation to provide rapid plasma concentrations or a rapid onset of effect or a loading dose and the inner capsule contains an extended release formulation. In some preferred embodiments, up to 3 capsules within a capsule are contemplated as part of the invention. In one embodiment of the invention, the dosage form involves one or more tablets within a capsule, wherein the buprenorphine is either in the tablet and/or in one of the capsules.

In one embodiment of the invention, the formulation is ingested orally as a tablet or capsule, preferably as a capsule. In another embodiment of the invention, the formulation is administered bucally. In yet another embodiment of the invention, the formulation is administered sublingually.

“Therapeutically effective amount” or “therapeutically-effective” refers to the amount of an active agent sufficient to induce a desired biological result. That result may be alleviation of the signs, symptoms, or causes of a disease, or any other desired alteration of a biological system.

“Therapeutically effective amount of buprenorphine” refers to the amount of oral buprenorphine sufficient to prevent, to cure, or at least partially arrest a medical disorder, disease, sign or symptom for which the buprenorphine has been prescribed to a subject.

The term “effective amount” means the quantity of a compound according to the invention necessary to prevent, to cure, or at least partially arrest a medical disorder, disease, sign or symptom for which the buprenorphine has been prescribed to a subject.

The term “pharmaceutically acceptable salt” as used herein refers to a salt which is toxicologically safe for human and animal administration. Nonlimiting examples of salts include hydrochlorides, hydrobromides, hydroiodides, sulfates, bisulfates, nitrates, citrates, tartrates, bitartrates, phosphates, malates, maleates, napsylates, fumarates, succinates, acetates, terephlhalates, pamoates and pectinates. In some embodiments, the pharmaceutical composition is a salt or complex of inorganic cation salts, organic salts such primary, secondary, tertiary and quaternary amines include substituted amines In some embodiments, examples of suitable pharmaceutically acceptable salts of buprenorphine include any of the inorganic cation salts such as sodium, potassium, lithium, magnesium, calcium, cesium, ammonia, ferrous, zinc, manganous, aluminum, ferric, and manganic; organic salts with primary, secondary, tertiary and quaternary amines, or mixtures thereof. Examples of such primary, secondary, tertiary and quaternary amines include substituted amines including but not limited to naturally occurring substituted amines, cyclic amines, basic ion exchange resins, and mixtures thereof. More specifically, suitable amines include but are not limited to tromethamine, triethylamine, tripropylamine, dropopizine, 2-dimethylaminoethanol, 2-diethylaminoethanol, lysine, arginine, ornithine, histidine, caffeine, procaine, N-ethylpiperidine, hydrabamine, choline, betaine, ethylenediamine, glucosamine, tris-(hydroxymethyl)aminomethane, N-methylglucamine, methylglycamine, theobromine, piperazine, piperidine, polyamine resins and the like, and mixtures thereof. In some embodiments, examples of suitable pharmaceutically acceptable salts of buprenorphine include aminoalcohols chosen from the group consisting of ethanolamine, 3-amino-1-propanol, (R)-1-amino-2-propanol, (S)-1-amino-2-propanol, 2-amino-1,3-propandiol, N-(2-hydroxyethyl)pyrrolidine, D-glucamine and L-prolinol, D-glucosamine, and N-methylglucosamine. In some embodiments, examples of suitable pharmaceutically acceptable salts of buprenorphine include alkali and alkaline earth metals and salts of an organic nature, such as the salts of basic amino acids.

In some embodiments of the invention, the term “controlled release” is interchangeable with “extended release”, “sustained release”, “modified release”, “delayed release” and the like.

It is contemplated that the present invention may be used alone or in combination with other drugs to provide additive, complementary, or synergistic therapeutic effects or for the treatment of entirely different medical conditions.

Other pharmaceutically active ingredients from various therapeutic classes may also be used in combination with the present invention. In some embodiment, co-administered may be used to provide additive, complementary, superadditive or synergistic therapeutic effects. In some embodiment, co-administered may be used to provide a different therapeutic effects from the present invention or to treat the side effects of the present invention. They include, but are not limited to decongestants, analgesics, analgesic adjuvants, antihistamines, expectorants, antitussives, diuretics, anti-inflammatory agents, antipyretics, antirheumatics, antioxidants, laxatives, proton pump inhibitors, motility modifying agents, vasodilators, inotropes, beta blockers, beta adrenergic agonists, drugs to treat asthma and COPD, antiinfectives, antihypertensives, antianginal agents, anticoagulants, lipid and cholesterol lowering drugs, anti-diabetic drugs, hormones, smooth muscle relaxants, skeletal muscle relaxants, bronchodilators, vitamins, trace minerals, amino acids, biological peptides, and drugs to treat disorders, diseases and maladies, and signs and symptoms thereof referred to in Harrison's Principles of Internal Medicine, 16th Edition, 2004, Kasper D L, Braunwald W, Fauci A, Hauser S, Longo D, and Jameson J L (eds)], which is hereby incorporated in its entirety by reference The drug being used in combination therapy with the present invention can be administered by any route, including parenterally, orally, topically, transdermally, sublingually, and the like.

The terms “medical condition”, “malady”, “disease”, “disorder” and “pathological states” are used interchangeably and are intended to have their broadest interpretation to refer to any physiologic, pathologic or pathophysiologic state in a human or other mammal that can be prevented, treated, managed or altered to produce a desired, usually beneficial effect.

In some embodiments, the oral buprenorphine is intended to prevent or treat pain. A co-administered drug (in the same or different dosage form, by any route of administration) may be used to provide additive, complementary, superadditive or synergistic therapeutic analgesic effects, including other NSAIDs, NO-NSAIDs, COX-2 selective inhibitors, acetaminophen, tramadol, local anesthetics, antidepressants, beta adrenergic agonists, alpha-2 agonists, selective prostanoid receptor antagonists, cannabinoid agonists, other opioid receptor agonists, NMDA receptor antagonists, gabapentin, pregabalin, gabapentinoids, neuronal nicotinic receptor agonists, calcium channel antagonists, sodium channel blockers, superoxide dismutase mimetics, p38 MAP kinase inhibitors, TRPV1 agonists, dextromethorphan, dextrorphan, ketamine, glycine receptor antagonists, antiepileptics, and any other drugs that can be shown by a person proficient in the art to prevent or treat pain.

In other embodiments, particularly preferred combinations include buprenorphine with acetaminophen.

In other embodiments, particularly preferred combinations include buprenorphine with an NSAID. Nonsteroidal anti-inflammatory drugs typically have analgesic, anti-inflammatory, and antipyretic properties. Their mode of action appears to involve inhibition of cyclooxygenases (COX-1 and COX-2), leukotriene biosynthesis, and antibradykinin activity. NSAIDs may be non-selective (inhibit COX-1 and COX-2 isozymes) or COX-2 selective (preferentially inhibit the COX-2 isozymes). Non-limiting examples of NSAIDs or COX-2 selective inhibitor include ibuprofen, tiaprofenic acid, diclofenac, piroxicam, loxoprofen, fenoprofen, indoprofen, oxaprozin, tenoxicam, lornoxicam, acetylsalicylic acid, mefenamic acid, naproxen, flurbiprofen, flubufen, ketoprofen, indoprofen, carprofen, pramoprofen, muroprofen, trioxaprofen, aminoprofen, tiaprofenic acid, fluprofen, niflumic acid, tolfenamic acid, diflunisal, etodolac, fenbufen, indomethacin, isoxicam, sudoxicam, pirprofen, sulindac, tolmetin, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflunisal, flufenisal, meloxicam and nabumetone, celecoxib, valdecoxib, etoricoxib, rofecoxib, and lumiracoxib, and as well as their pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In other embodiments, particularly preferred combinations include buprenorphine with NMDA antagonists.

The expression “N-methyl-D-aspartate receptor” shall be understood to include all of the binding site subcategories associated with the NMDA receptor, e.g., the glycine-binding site, the phenylcyclidine (PCP)-binding site, etc., as well as the NMDA channel. Thus, the invention herein contemplates the use of nontoxic substances that block an NMDA receptor binding site. NMDA receptor antagonists are substances known to those skilled in the art that block the N-methyl-D-aspartate receptor that block a major intracellular consequence of NMDA receptor activation, see U.S. Pat. Nos. 5,321,012; 5,654,281 and 5,869,498, all of which are hereby incorporated by reference in their entireties, and particularly for the purpose of describing NMDA receptor antagonists and their uses. The NMDA receptor antagonist may be a mixture. Non-limiting examples of preferred NMDA receptor antagonists include dextromethorphan ((+)-3-hydroxy-N-methylmorphinan), its metabolite dextrorphan ((+)-3-hydroxy-N-methylmorphinan), amantadine (1-amino adamantine), memantine (3,5dimethylaminoadamantone), amitriptyline, D,L-2-amino-5-phosphono valeric acid, ketamine, methadone, racemorphan, levorphanol, and as well as their pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In other embodiments, particularly preferred combinations include buprenorphine with antiepileptics.

Non-limiting examples of anti-epileptic compounds include gabapentin, pregabalin, carbamazepine, oxcarbazepine, lamotrigine, phenyloin, fosphenyloin, valproate, valproic acid, tiagabine, topiramate, divalproex, harkoseride, and levetiracetam, in unsalified form or as pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In some preferred embodiment of the present invention, anti-epileptic compounds having pain alleviating properties include those that have the following Formula I

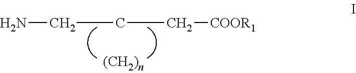

wherein R₁ is hydrogen or a lower alkyl, n is an integer of from 4 to 6; and the cyclic ring is optionally substituted, and the pharmaceutically acceptable salts thereof. The term lower alkyl includes straight or branched chain alkyl groups of up to eight carbon atoms. An especially preferred embodiment utilizes a compound of Formula I where R₁ is hydrogen and n is 5, which compound is 1-(aminomethyl)cyclohexane acetic acid, known generically as gabapentin.

Other preferred compounds of Formula I above include, but are not limited to: ethyl 1-aminomethyl-1-cyclohexaneacetate; 1-amino methyl-1-cycloheptane-acetic acid; 1-aminomethyl-1-cyclopentane-acetic acid; methyl-laminomelhyl-1-cyclohexane-acetate; n-butyl 1-aminomethyl-1-cyclohexaneacetate; methyl 1-aminomethyl-1-cycloheptaneacetate; n-butyl 1-aminomethyl-1-cycloheptane-acetate toluene sulfonate; 1-aminomethyl-1-cyclopentaneacetate benzene-sulfonate; and n-butyl 1-aminomethyl-1-cyclopentane-acetate.

Other preferred compounds of Formula I above, wherein the cyclic ring is substituted for example with alkyl such as methyl or ethyl, include, but are not limited to (1-aminomethyl-3-methylcyclohexyl)acetic acid, (1-aminomethyl-3-methylcyclopentyl)acetic acid, and (1-aminomethyl-3,4-dimethylcyclopentyl)acetic acid.

In another preferred embodiment of the present invention, anti-epileptic compounds having pain alleviating properties include those that are included in Formula II:

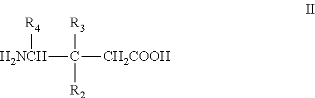

wherein R₂ is a straight or branched alkyl of from 1 to 6 carbon atoms, phenyl, or cycloalkyl having from 3 to 6 carbon atoms, R₃ is hydrogen or methyl, and R₄ is hydrogen, methyl, or carboxyl, or an individual diastereomeric or enantiomeric isomer thereof or a pharmaceutically acceptable salt thereof.

The most preferred compound of Formula II is where R₃ and R₄ are both hydrogen and R₂ is —(CH₂)ₘ-iC₄H₉ as an (R), (S) or (R,S) isomer, wherein m is 0 to 2. A more preferred embodiment of the invention utilizes 3-aminomethyl-5-methyl-hexanoic acid, and especially (S)-3-(aminomethyl)-5-methylhexanoic acid, now known generically as pregabalin. Another preferred compound is 3-(1-aminoethyl)-5-methylhexanoic acid.

In other embodiments, particularly preferred combinations include buprenorphine with antidepressants.

Antidepressants are well known in the art. Non-limiting examples of antidepressants include drugs from the following classes: tricyclic antidepressants, tetaracyclic antidepressants, SRI's, SSRI's, SNRI's and NSRI's. Non-limiting examples of specific antidepressants include amitriptyline, bupropion, citalopram, protriptyline, nortriptyline, desipramine, doxepin, imipramine, clomipramine, fluoxetine, paroxetine, sertraline, venlafaxine, duloxetine, trazodone, nefazodone, maprotiline and mirtazpine in unsalified form or as pharmaceutically acceptable salts, prodrugs, esters, analogs, derivatives, solvates, complexes, polymorphs, hydrates and metabolites, as racemates or an individual diastereoisomers or enantiomeric isomers thereof or mixture thereof.

In other embodiments, particularly preferred combinations include buprenorphine with calcium channel blockers.

Calcium channel blockers are well or can be readily determined by established methods. Included calcium channel blockers are those disclosed or referenced in U.S. Pat. Nos. 7,064,128, 6,989,448, 6,951,862, 6,951,860, 6,949,554, 6,946,475, 6,852,742, 6,818,200, 6,617,322, 6,541,479, 6,495,715, 6,492,375, 6,469,038, 6,423,689, 6,387,897, 6,334,997, 6,310,059, 6,294,533, 6,267,945, 6,251,919, 6,251,918, 6,221,335, 6,191,151, 6,166,052, 6,124,280, 6,117,841, 6,106,856, 6,090,631, 6,011,035, 5,981,539, 5,932,573, 5,886,012, 5,767,129, 5,698,549, 5,623,051, 5,492,904, 5,484,789, 5,457,132, 5,360,809, 5,354,765, 5,350,771, 5,314,903, 5,160,734, 5,132,119, 5,070,088, 4,963,671, 4,880,824, 4,833,162, 4,808,718 and 4,791,117 and in United States patent application No. 20060084660, 20060063775, 20050227999, 20050197351, 20050191245, 20050165065, 20050159455, 20050153953, 20050014748, 20040259866, 20040253300, 20040209872, 20040204404, 20040192703, 20040147529, 20040028734, 20040006110, 20030199523, 20030060632, 20030060419, 20030045530, 20020160995, 20020115655, 20020094995, 20010029258 and 20010023249, all of which are hereby incorporated by reference in their entirety.

In other embodiments, particularly preferred combinations include buprenorphine with sodium channel modulators.

Sodium channel modulators are well or can be readily determined by established methods. Included sodium channel modulators are those disclosed or referenced in U.S. Pat. Nos. 7,078,515, 7,067,629, 7,063,953, 7,041,704, 7,022,714, 6,994,993, 6,872,741, 6,828,311, 6,770,484, 6,756,400, 6,646,012, 6,559,154, 6,479,498, 6,479,259, 6,420,354, 6,335,172, 6,221,887, 6,184,349, 6,172,085, 6,030,810, 5,776,859, and 5,437,982 and in United States patent application No. 20060223117, 20060205738, 20060183897, 20060183785, 20060142581, 20060142306, 20060063780, 20060052395, 20060052394, 20060040954, 20060020006, 20060008541, 20050260576, 20050234072, 20050233957, 20050228182, 20050228033, 20050227974, 20050227270, 20050203190, 20050186627, 20050165072, 20050130966, 20050113390, 20050113389, 20050113388, 20050112633, 20050095225, 20050090547, 20050089559, 20050080092, 20050080091, 20050059676, 20050037442, 20050020564, 20040265788, 20040248240, 20040248207, 20040229884, 20040220170, 20040213842, 20040204460, 20040204425, 20040204424, 20040198749, 20040198748, 20040198747, 20040198746, 20040198745 and 20040198744 all of which are hereby incorporated by reference in their entirety.

In other embodiments, particularly preferred combinations include buprenorphine with cannabinoid agonists.

The term “cannabinoid agonist” means a substance that binds to one or more cannabinoid receptor to exert an agonist or partial agonist effect.

A number of assays are available to determine whether a drug is a cannabinoid agonist, using in vivo and in vitro bioassay systems (Howlett et al., Mol Pharmacol, 1988; International Union of Pharmacology [IUPHAR], http://www.iuphar.org/index.html; Subcommittees on Cannabinoid Receptors The International Committee of Pharmacology Committee on Receptor Nomenclature and Classification [NC-IUPHAR], http://www.iuphar.org/nciuphar.html)

Cannabinoid agonists are known or readily determined by individuals who practice the art. Preferably, the cannabinoid agonist useful for the present invention may be selected from the group consisting of THC, THC analogs and derivatives, cannabidiol, 9-THC propyl analog, cannabidiol, cannabidiol propyl analog, cannabinol, cannabichromene, cannabichromene propyl analog, cannabigerol, cannabinoid terpenoids, cannabinoid flavonoids, endocannabinoids, anandamide and 2-arachidonoylglycerol, THC-like ABC tricyclic cannabinoid analogues, exemplified by HU210 and desacetyllevonantradol; synthetic AC bicyclic and ACD tricyclic cannabinoid analogues, exemplified by CP55940, and CP55244 and aminoalkylindole compounds, exemplified by WIN55212-2 (Little et al., Pharmacol. Biochem. Behav, 1989; Howlett et al., Neuropharmacology, 1990; Johnson et al, In: Cannabinoids as Therapeutic Agents (Mechoulam, R., ed.), CRC Press, 1986; Howlett et al., Mol Pharmacol, 1988; D'Ambra et al., J Med Chem, 1992; Pacheco et al., J Pharmacol Exp Ther, 1991; Compton et al, J Pharmacol Exp Ther, 1992; Howlett et al, Pharmacol Rev, 2002).

Preferably, the cannabinoid agonist may be selected from compounds disclosed or referenced in U.S. Pat. Nos. 7,071,213, 7,067,539, 7,057,051, 7,037,910, 6,995,187, 6,977,266, 6,949,582, 6,943,266, 6,930,122, 6,916,838, 6,914,072, 6,903,137, 6,864,291, 6,864,285, 6,790,365, 6,653,304, 6,642,258, 6,563,009, 6,525,087, 6,509,367, 6,475,478, 6,448,288, 6,403,126, 6,344,474, 6,328,992, 6,284,788, 6,113,940, 6,100,259, 5,990,170, 5,948,777, 5,939,429, 5,925,768, 5,872,148, 5,817,766, 5,747,524, 5,605,906, 5,596,106, 5,532,237, 5,440,052, 4,816,415 and in U.S. patent Application No. 20060172019, 20060160850, 20060160776, 20060155126, 20060154958, 20060154956, 20060154955, 20060128738, 20060128673, 20060122230, 20060115816, 20060100228, 20060100208, 20060094774, 20060094123, 20060089378, 20060089356, 20060084659, 20060079556, 20060052411, 20060030563, 20060009528, 20050282861, 20050267161, 20050250769, 20050239828, 20050239133, 20050228034, 20050192278, 20050187253, 20050171110, 20050165118, 20050143381, 20050137197, 20050137173, 20050096379, 20050095674, 20050080087, 20050065216, 20050065189, 20050054659, 20050026986, 20050020544, 20050014786, 20050009903, 20050009870, 20040259887, 20040248956, 20040248881 and 20040242593.

The term “cannabinoid receptor” means a molecule that causes a specific physiologic, pathophysiologic or pharmacologic effect after binding to CB₁, CB₂, non-CB₁/CB₂ cannabinoid sites, TRPV₁ receptors, as well as other G protein-coupled receptors (GPCRs) that form part of the endocannabinoid system (Wiley and Martin, Chemistry Physics of Lipids, 2002; Begg et al., Pharmacol Ther, 2005; Howlett et al., Neuropharmacol, 2004; Pertwee, AAPS Journal, 2005; International Union of Pharmacology (IUPHAR) Receptor Database; Howlett et al., Mol Pharmacol, 1988; International Union of Pharmacology [IUPHAR], http://www.iuphar.org/index.html; Subcommittees on Cannabinoid Receptors The International Committee of Pharmacology Committee on Receptor Nomenclature and Classification [NC-IUPHAR], http://www.iuphar.org/nciuphar.html).

Notwithstanding the above definitions, for the purposes of the present invention, drugs that enhance the effect of cannabinoid agonists by inhibiting their metabolism or reuptake (for example, anandamide amidase inhibitors) are also considered to be cannabinoid agonists.

In other embodiments, particularly preferred combinations include buprenorphine with other opioids.

Opioid agonists include alfentanil, allylprodine, alphaprodine, anileridine, apomorphine, apocodeine, benzylmorphine, bezitramide, buprenorphine, butorphanol, carfentanil, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, nociceptin/orphanin FQ (N/OFQ), normorphine, norpipanone, ohmefentanyl, opium, oxycodone, oxymorphone, papavereturn, pentazocine, phenadoxone, phenomorphan, phenazocine, phenoperidine, pholcodine, piminodine, piritramide, propheptazine, promedol, profadol, properidine, propiram, propoxyphene, racemorphan, remifentanil, sufentanil, tapentadol, tramadol, tilidine, methylnaltrexone, naloxone methiodide, naloxonazine, nalmexone, nalbuphine, nalorphine dinicotinate, naltrindole (NTI), naltrindole isothiocyanate, (NTII), naltriben (NTB), nor-binaltorphimine (nor-BNI), beta-funaltrexamine (b-FNA), BNTX, cyprodime, ICI-174,864, LY117413, MR2266, etorphine, DAMGO, CTOP, diprenorphine, naloxone benzoylhydrazone, bremazocine, ethylketocyclazocine, U50,488, U69, 593, spiradoline, DPDPE, [D-Ala2,Glu4]deltorphin, DSLET, Met-enkephalin, Leu-enkephalin, (3-endorphin, dynorphin A, dynorphin B, a-neoendorphin, or an opioid having the same pentacyclic nucleus as nalmefene, naltrexone, buprenorphine, levorphanol, meptazinol, pentazocine, dezocine, or their pharmaceutically acceptable salts, prodrugs, esters, analogs, derivatives, solvates, complexes, polymorphs, hydrates and metabolites, as racemates or an individual

In other embodiments, particularly preferred combinations include buprenorphine with muscle relaxants, including cyclobezaprine and drugs selected from the class of benzodiazepine agonists (e.g, diazepam, lorazepam, tamazepam, nitrazepam, flurazepam, etc).

In certain preferred embodiments of the present invention, an effective amount of another drug to treat the buprenorphine responsive condition, a buprenorphine related side effect (e.g., laxative, CNS stimulant or anti-emetic) or a co-existing medical condition may be incorporated into the dosage form. Such a coadmistered drug may be in any form, including immediate release, controlled release and delayed release. The co-adminstered drug may be incorporated at a therapeutic dose or a subtherapeutic dose. If included in immediate release form, it may be coated onto the substrates of the present invention. For example, where the extended release buprenorphine from the formulation is due to a controlled release coating, the immediate release layer of another drug may be overcoated on top of the controlled release coating. On the other hand, the immediate release layer maybe coated onto the surface of substrates wherein the buprenorphine is incorporated in a controlled release matrix. Where a plurality of the sustained release substrates comprising an effective unit dose of the buprenorphine (e.g., multiparticulate systems including pellets, spheres, beads and the like) are incorporated into a capsule, the immediate release drug may be incorporated into the capsule via inclusion of the sufficient amount of immediate release drug as a powder or granulate within the capsule. Alternatively, the capsule itself may be coated with an immediate release layer of the drug. One skilled in the art would recognize still other alternative manners of incorporating the immediate release buprenorphine into the unit dose. Such alternatives are deemed to be encompassed by the appended claims. By including such an effective amount of immediate release drug to treat the same condition as the buprenorphine, it may be possible to reduce the dose of buprenorphine in the dosage form.

In certain preferred embodiments of the present invention where the dosage form is delayed onset (e.g, delayed onset, rapid release; delayed onset, extended release; or delayed onset, pulsatile release), an effective amount of another drug to treat the buprenorphine responsive condition in immediate release form may be particularly advantageous. In certain preferred embodiments, an NSAID, acetaminophen or a COX-2 inhibitor in immediate release form may be advantageously incorporated into the dosage form.

In some embodiments, another drug to treat the same condition as the oral buprenorphine or to treat a different condition may be incorporated into the oral dosage form, where the other drug shares one, or more, or all the dissolution rate specifications, GI delivery and release specifications and pharmacokinetic parameter specifications (limited to Tₘₐₓ, HVD and W₅₀) as the oral buprenorphine in the dosage form.

In some embodiments, another drug to treat the same condition as the oral buprenorphine or to treat a different condition may be incorporated into the oral dosage form, where the other drug has different dissolution rate specifications, GI delivery and release specifications and pharmacokinetic parameter specifications from the oral buprenorphine in the dosage form, one or more or all said different specifications contained herein.

In some embodiments, another drug to treat the same condition as the oral buprenorphine or to treat a different condition may be incorporated into the oral dosage form, where the other drug has different dissolution rate specifications, GI delivery and release specifications and pharmacokinetic parameter specifications from the oral buprenorphine in the dosage form.

### BRIEF DESCRIPTION OF THE DRAWINGS/FIGURES

The included drawings are illustrative but not limiting of the methods and composition of the present invention. Other suitable modifications and adaptations of the variety of conditions and parameters normally encountered and obvious to those skilled in the art are within the spirit and scope of the invention.

FIG. 1: Time-effect curves depicting antinociceptive effect of orally administered buprenorphine in the tail flick test. The percent maximum possible effect (% MPE) is plotted versus time.

FIG. 2: Dose-response curves depicting the antinociceptive effect of buprenorphine administered orally in the tail flick test. The percent area under (analgesic effect) curve (AUC) from the time-effect curves is plotted versus log [dose].

FIG. 3: Time-effect curves depicting antinociceptive effect of orally administered buprenorphine in the hotplate test. The percent maximum possible effect (% MPE) is plotted versus time.

FIG. 4: Dose-response curves depicting the antinociceptive effect of buprenorphine administered orally in the hot plate test. The percent area under (analgesic effect) curve (AUC) from the time-effect curves is plotted versus log [dose].

FIG. 5: Illustrates the gastrointestinal tract of a human subject, including the stomach, the small intestine (duodenum, jejunum and ileum), and the colon.

FIG. 6: Provides an expanded view of the anatomy of the colon.

FIG. 7: Illustrates the average pH of various segments of the gastrointestinal tract.

FIG. 8: Illustrates the average pH of for dissolution of many pH sensitive polymers in the various segments of the gastrointestinal tract.

FIG. 9: Illustrates the average gastrointestinal transit times for various segments of the gastrointestinal tract.

FIG. 10: Illustrates the in vivo release of a delayed onset, rapid release dosage form of oral buprenorphine containing an ileo-colonic pH sensitive polymer. The dosage form resists release of the buprenorphine at pH less than 5 for a prolonged period of time and releases the contents of the dosage form at a pH greater than 6.5.

FIG. 11: Illustrates the in vivo release of a delayed onset, rapid release dosage form of oral buprenorphine containing an ileo-colonic pH sensitive polymer. The dosage form resists release of the buprenorphine at pH less than 5.5 for a prolonged period of time and releases the contents of the dosage form at a pH greater than 7.

FIG. 12: Illustrates the in vivo release of a delayed onset, extended release dosage form of oral buprenorphine upon reaching or traversing the ileo-cecal junction and transiting into the colon.

FIG. 13: Illustrates the in vivo release of a delayed onset, extended release dosage form of oral buprenorphine upon reaching or traversing the ileo-cecal junction and transiting into the colon.

FIG. 14: Illustrates a dosage form for colonic release: (1) the outer layer which dissolves at a pH of about 7; (2) a sustained release polymer coating; (3) the buprenorphine which has been coated onto a nonpareil core or bead (4).

FIG. 15: Illustrates a delayed onset, rapid release dosage form for ileo-colonic or colonic release. Following a lag period during which no buprenorphine or very little no buprenorphine is released in vivo, the dosage form upon reaching a certain GI environment (e.g., desired pH, pressure, enzymes, microbial flora) or time, or a combination of variables, releases the dosage form in a rapid (albeit) pulse or burst.

FIG. 16: Illustrates a delayed onset, extended release dosage form for ileo-colonic or colonic release. Following a lag period during which no buprenorphine or very little buprenorphine is released in vivo, the dosage form upon reaching a certain GI environment (e.g., desired pH, pressure, enzymes, microbial flora) or time, or a combination of variables, releases the dosage form in an extended release form.

FIG. 17: Time-effect curves depicting antinociceptive effect of intra-gastric, intra-ileal and intra-colonic buprenorphine in the tail flick test. The percent maximum possible effect (% MPE) is plotted versus time. Top curve (intra-colonic, triangle), second from top curve (intra-ileal, large square) and bottom curve (intra-gastric, small diamond).

FIG. 18: Time-effect curves depicting antinociceptive effect of intra-gastric, intra-ileal and intra-colonic buprenorphine in the tail flick test. The percent maximum possible effect (% MPE) is plotted versus time. Top curve (intra-colonic, triangle), second from top curve (intra-ileal, large square) and bottom curve (intra-gastric, small diamond).

FIG. 19: Time-effect curves depicting the analgesic effect of the orally administered buprenorphine in the von Frey hair test of mechanical threshold of pain in rats with vincristine induced peripheral neuropathy. The mechanical threshold of pain (g) are plotted versus time]

FIG. 20: Time-effect curves depicting the analgesic effect of the orally administered buprenorphine in the acetone drop test of thermal allodynia as assessed in rats with vincristine induced peripheral neuropathy. The percent maximum possible effect (% MPE) is plotted versus time.

FIG. 21: Time-effect curves depicting the analgesic effect of the orally administered buprenorphine in the von Frey hair test of mechanical threshold of pain in rats with paclitaxel induced peripheral neuropathy. The mechanical threshold of pain (g) are plotted versus time]

FIG. 22: Time-effect curves depicting the analgesic effect of the orally administered buprenorphine in the acetone drop test of thermal allodynia as assessed in rats with paclitaxel induced peripheral neuropathy. The percent maximum possible effect (% MPE) is plotted versus time.

FIG. 23: Time-effect curves depicting the analgesic effect of the orally administered buprenorphine in the von Frey hair test of mechanical threshold of pain in rats with spinal nerve ligation induced peripheral neuropathy. The mechanical threshold of pain (g) are plotted versus time]

FIG. 24: Time-effect curves depicting the analgesic effect of the orally administered buprenorphine in the acetone drop test of thermal allodynia as assessed in rats with spinal nerve ligation induced neuropathy. The percent maximum possible effect (% MPE) is plotted versus time.

FIG. 25: Time-effect curves depicting the analgesic effect of the orally administered buprenorphine in the von Frey hair test of mechanical threshold of pain in rats with streptozotocin (STZ) induced diabetes neuropathy. The mechanical threshold of pain (g) are plotted versus time]

FIG. 26: Time-effect curves depicting the analgesic effect of the orally administered buprenorphine in the acetone drop test of thermal allodynia as assessed in rats with streptozotocin (STZ) induced diabetes neuropathy. The percent maximum possible effect (% MPE) is plotted versus time.

FIG. 27: Frequency of flinching response in the formalin model of persistent pain after intra-gastric, intra-ileal and intra-colonic buprenorphine, compared with untreated controls.

### METHODS OF CARRYING OUT THE INVENTION

### Dosage Forms

Pharmaceutical composition and methods of the present invention contain buprenorphine base or pharmaceutically acceptable salts in racemic or enantiomeric form, or mixtures thereof intended for oral administration.

All oral pharmaceutical dosage forms of the invention are contemplated, including oral suspensions, tablets, chewable tablets, capsules, lozenges, effervescent tablets, effervescent powders, powders, solutions, powders for reconstitution, gastroretentive tablets and capsules, orally disintegrating tablets, oral fast dissolving tablets, oral fast dispersing tablets, oral fast disintegrating dosage forms, each administered as immediate release, modified release, enteric coated, sustained release, controlled release, pulsatile release or extended release dosage form.

The formulation may optionally comprise excipients, including release controlling excipients and non-release controlling excipient. Non-limiting examples of these auxiliary materials (or pharmaceutically acceptable excipients) are (i) Binders such as acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like; (ii) Disintegrants such as starches, pregelatinized corn starch, pregelatinized starch, celluloses, cross-linked carboxymethylcellulose, crospovidone, cross-linked polyvinylpyrrolidone, a calcium or a sodium alginate complex, clays, alginates, gums, or sodium starch glycolate, and any disintegration agents used in tablet preparations; (iii) Filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like; (iv) Stabilizers such as any antioxidation agents, buffers, or acids, and the like; (v) Lubricants such as magnesium stearate, calcium hydroxide, talc, colloidal silicon dioxide, sodium stearyl fumarate, hydrogenated vegetable oil, stearic acid, glyceryl behenate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like; (vi) Wetting agents such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like; (vii) Diluents such lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like; (viii) Anti-adherents or glidants such as talc, corn starch, DL-leucine, sodium lauryl sulfate, and magnesium, calcium, or sodium stearates, and the like; (ix) Pharmaceutically compatible carriers such as acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, or pregelatinized starch, and the like; and (x) excipients referred to herein.

### Targeted Gastrointestinal Delivery

Targeted delivery of the buprenorphine dosage form of the present invention for release and subsequent absorption can be achieved to provide delayed onset, rapid release dosage forms; delayed onset, pulsatile release dosage forms; delayed onset, extended release dosage forms; and other modified release dosage forms. In addition, conventional extended release products which release the active drug rapidly on ingestion may be coated or embedded with further controlled release material designed to provide a lag time before release of drug upon ingestion.

A wide variety of methods for the preparation of delayed onset dosage form are known in the art. These methods may be employed for the preparation of delayed onset dosage forms of the invention, including but not limited to: (i) Prodrug Approach: in some embodiments such products control the rate of release of active drug by azo-bond conjugates, glycoside conjugates, glucuronide conjugates, cyclodextrin conjugates, dextran conjugates, polypeptide conjugates; (ii) Polymeric Coating: in some embodiments, such products control the release of active drug by coating with pH sensitive polymers and coating with biodegradable polymers; (iii) Embedding in Matrices: in some embodiments, such products control the release of active drug by embedding in pH sensitive matrices, embedding in biodegradable hydrogels and matrices (e.g., amylose, chondroitin sulfate, chitosan, inulin, dextran, guar gum, pectin). Other approaches include the use of time dependent systems, Pulsincap®, CODDES® and intestinal pressure controlled delivery systems.

In some embodiments, the need for a rapid initial dose may require that a portion of the dose (e.g., up to about 1%, or 3%, or 5%, or 7%, or 10%, or 12%, or 15%, or 17%, or 20%, or 22%, or 25%, or 30%) is provided without delay as an immediate release dosage form (e.g., without limitation, a capsule within a capsule, a tablet within a capsule, an immediate release overcoat on a tablet or a capsule) in order to achieve, for example, immediate symptom relief

An immediate or controlled release tablet or capsule formulation may be overcoated with one or more polymers to provide buprenorphine release in the appropriate gastrointestinal environment (defined, in some embodiments by location in the GI tract, pH at the point of release, osmotic pressure at the point of release, hydration, microbial flora, and/or the time after ingestion at the point of release).

In some embodiments, the dosage form of the invention is in the form of a compressed tablet, or a capsule, said tablet or capsule coated with a polymer to retard or delay its release to achieve the objectives of the invention, said polymers including polymethacrylates (copolymerisate of methacrylic acid and either methylmethacrylate or ethyl acrylate (Eudragit®), cellulose based polymers e.g. cellulose acetate phthalate (Aquateric®) or polyvinyl derivatives e.g. polyvinyl acetate phthalate (Coateric®).

In some embodiments, the dosage form of the invention is in the form of a compressed tablet or a capsule, said tablet or capsule coated with one or more anionic polymers with methacrylic acid as a functional group (Eudragit™ polymer, Evonik Degussa, Darmstadt, Germany) to retard or delay its release to achieve the objectives of the invention, said polymers including Eudragit™ L 30 D-55 or Eudragit™ L 100-55 which dissolve in the duodenum or at about pH>5.5, or Eudragit™ L 100 which dissolves in the jejunum or at a pH of about 6, or Eudragit™ S100, which dissolves in the ileum or at a pH o>7.0, or Eudragit™ FS 30D, which dissolves in the colon or at a pH of about 6, which dissolve at a pH>7.0.

In some embodiments, the dosage form of the invention can provide delayed and subsequently ileo-colonic or colonic release over an extended period of time (an extended release) by use of multiple polymers. In one embodiment, at the center of the dosage form is a core containing the buprenorphine. The buprenorphine is then coated with one or more layers of polymers that permit the drug to pass through the stomach, duodenum and jejunum (and optionally the ileum) without substantial absorption. As the dosage form reaches the alkaline pH of the ileum and colon (or optionally, upon arrival near or in the colon, for example upon traversing the ileo-cecal junction), the polymer allows permeability to water and thereby allows drug to diffuse from the dosage form and be available for systemic absorption in the terminal ileum and/or in the colon.

In some embodiments, the dosage form of the invention can provide delayed and subsequently rapid ileo-colonic or colonic release (an immediate pulsed release) by use of materials such as polymers. In this manner, after a defined lag time, rapid (pulsed) drug release is provided. For example, a layer composed of drug and organic acid is applied to the dosage form core. The core is then coated with material such a poly(meth)acrylate with basic groups such as quaternary ammonium groups, which become permeable in the presence of water. During gastrointestinal transit, water penetrates the coating into the core and dissolves the organic acid. Following the desired lag time, the drug is released as an immediate liberation or pulsed release.

In some embodiments, the dosage form of the invention is in the form of a capsule, said capsule made from materials known in the art, including gelatin, plasticizers, starch, hydroxypropylmethyl cellulose (HPMC), pullulan capsule.

In some embodiments of the invention, the buprenorphine if formulated as an immediate release or controlled release tablet or capsule formulation. If used as prepared, the dosage form would usually release some of the buprenorphine from the dosage form in the stomach, duodenum, jejunum and ileum. Some suitable coatings include U.S. Pat. Nos. 4,311,833; 4,377,568; 4,385,078; 4,457,907; 4,462,839; 4,518,433; 4,556,552; 4,606,909; 4,615,885; 4,670,287; 5,536,507; 5,567,423; 5,591,433; 5,597,564; 5,609,871; 5,614,222; 5,626,875; 5,629,001; and 6,608,075, all of which are incorporated herein in their entirety by reference.

In some embodiments of the invention, preferred coating compositions include alkyl and hydroxyalkyl celluloses and their aliphatic esters, e.g., methylcellulose, ethylcellulose, hydroxyethylcellulose, hydroxypropylcellulose, hydroxybutylcellulose, hydroxyethylethylcellulose, hydroxyprophymethylcellulose, hydroxybutylmethylcellulose, hydroxypropylcellulose phthalate, hydroxypropylmethylcellulose phthalate and hydroxypropylmethylcellulose acetate succinate; carboxyalkylcelluloses and their salts, e.g., carboxymethylethylcellulose; cellulose acetate phthalate; cellulose acetate trimellitate, polycarboxymethylene and its salts and derivatives; polyvinyl alcohol and its esters: polyvinyl acetate phthalate; polycarboxymethylene copolymer with sodium formaldehyde carboxylate; acrylic polymers and copolymers, e.g., methacrylic acid-methyl methacrylic acid copolymer and methacrylic acid-methyl acrylate copolymer; edible oils such as peanut oil, palm oil, olive oil and hydrogenated vegetable oils; polyvinylpyrrolidone; polyethylene glycol and its esters: natural products such as shellac, and zein.

In some embodiments of the invention, other preferred coatings include polyvinylacetate esters, e.g., polyvinyl acetate phthalate; alkyleneglycolether esters of copolymers such as partial ethylene glycol monomethylether ester of ethylacrylate-maleic anhydride copolymer or diethyleneglycol monomethylether ester of methylacrylate-maleic anhydride copolymer, N-butylacrylate-maleic anhydride copolymer, isobutylacrylate-maleic anhydride copolymer or ethylacrylate-maleic anhydride copolymer; and polypeptides resistant to degradation in the gastric environment, e.g., polyarginine and polylysine. Other suitable coatings and methods to make and use delayed onset, rapid release and delayed onset, extended release, and delayed onset, pulsatile release, and controlled release, and modified release, and extended release, and pulsatile release, and slow release, and duodenal release, and jejunal release, and ileal release, and ileo colonic release and colonic release pharmaceutical compositions and dosage forms of oral buprenorphine are well known to those skilled in the art, including, Colonic Drug Delivery (page 287-294), Wilson C G, In: Modified-Release Drug Delivery Technology, Second Edition, Vol. 1, Rathbone M J, Hadgraft J, Roberts M S, Lane M E (eds), Informa Heakthcare USA Inc. 2008; Biopolymers and Colonic Delivery, Wilson C G, Mukherji G, Shah H K (pages 295-309), In: Modified-Release Drug Delivery Technology, Second Edition, Vol. 1, Rathbone M J, Hadgraft J, Roberts M S, Lane M E (eds), Informa Heakthcare USA Inc. 2008; Enteric Coating for Colonic Delivery, Shah H K, Mukherji G, Brogmann B, Wilson C G (pages 311-324), In: Modified-Release Drug Delivery Technology, Second Edition, Vol. 1, Rathbone M J, Hadgraft J, Roberts M S, Lane M E (eds), Informa Heakthcare USA Inc. 2008; Programmed Drug Delivery Systems and the Colon, Wilson C G, Shah H K, Lee W W, Brogmann B, Mukherji G (pages 325-335), In: Modified-Release Drug Delivery Technology, Second Edition, Vol. 1, Rathbone M J, Hadgraft J, Roberts M S, Lane M E (eds), Informa Heakthcare USA Inc. 2008; Targeting the Colon Using COLAL™: A Novel Bacteria-Sensitive Drug Delivery System, McConnell E L, Basit A W (pages 343-348), In: Modified-Release Drug Delivery Technology, Second Edition, Vol. 1, Rathbone M J, Hadgraft J, Roberts M S, Lane M E (eds), Informa Heakthcare USA Inc. 2008; Remington: the science of Pharmacy Practice, 21^{st} Edition, 2006, Lippincott, Williams & Wilkins, Baltimore, Md.; Pharmaceutical Preformulation and Formulation: A Practical Guide from Candidate Drug Selection to Commercial Dosage Form. Gibson, M (ed). CRC Press, 2001; Niazi, S. Handbook of Pharmaceutical Manufacturing Formulations: Compressed Solid Products (Volume 1 of 6), CRC Press, 2004; Niazi, S. Mollet, H, Grubenmann A, Payne H. Formulation Technology: Emulsions, Suspensions, Solid Forms, Wiley-VCH, 2001; FDA list and database; FDA Color Additive Status List; FDA Inactive Ingredients Database; Rowe, Sheskey and Owen, Handbook of Pharmaceutical Excipients, APhA Publications; 5th edition (2006); Rowe, Sheskey and Quinn, Handbook of Pharmaceutical Excipients, 6 edition, Pharmaceutical Press; APhA Publications; 2009; Pharmaceutical Additives Electronic Handbook, Third Edition, Michael Ash (compiler), Synapse Information Resources, Inc.; 3 Cdr edition (Feb. 19, 2007); and Health Canada's List of Acceptable Non-medicinal Ingredients; Patel et al. Therapeutic Opportunities in Colon-Specific Drug-Delivery System, Therapeutic Drug carrier Systems, 24(2), 147-202 (2007); Van den Mooter, Colon Drug Delivery, Expert Opin Drug Deliv (2006) 3(1):111-125; Singh. Modified-Release Solid Formulation for Colonic Delivery, Drug Deliv & Formulation 2007, 1, 53-56; Kumar et al. Colon Targeted Drug System—An Overview, Current Drug Deliv, 2008, 5, 186-198; Jain et al. Target-Specific Drug Release to the Colon, Expert Opin Drug Deliv (2008) 5(5): 483-498; Wei et al. Selective Drug Delivery to the Colon Using Pectin-Coated Pellets, PDA J Pharm Sci Tech Vol. 62, No. 4, 2008; Schellekens et al. Pulsatile Drug Delivery to Ileo-Colonic Segments by Structured Incorporation of Disintegrants in pH-Responsive Polymer Coatings, J Controlled Release 132 (2008) 91-98; Coviello et al. Polysaccharide Hydrogels for Modified Release Formulations, J Controlled Release 119 (2007) 5-24; McConnell et al. An in Vivo Comparison of Intestinal pH & Bacteria as Physiological Trigger Mechanisms for Colonic Targeting in Man, J Controlled Release 130 (2008) 154-160; Wei et al. Chitosan/Kollicoat SR 30D film-coated pellets of aminosalicylates for colonic drug delivery J Pharm Sci, 2009 Aug. 4; Yassin et al. New targeted-colon delivery system: in vitro and in vivo evaluation using X-ray imaging. J Drug Target, 2009 Aug. 5; Fan et al. Studies of chitosan/Kollicoat SR 30D film-coated tablets for colonic drug delivery. Int J Pharm, 2009 Jun. 22; 375 (1-2):8-15; Yehia et al. Optimization of budesonide compression-coated tablets for colonic delivery. AAPS PharmSciTech, 2009; 10(1):147-57; Chandran et al. Microspheres with pH modulated release: Design and characterization of formulation variables for colonic delivery J Microencapsul, 2008 Sep. 22:1-11; Gohel et al. Design of a potential colonic drug delivery system of mesalamine. Pharm Dev Technol 2008; 13(5):447-56; Maestrelli et al. Microspheres for colonic delivery of ketoprofen-hydroxypropyl-beta-cyclodextrin complex. Eur J Pharm Sci 2008 May 10; 34(1):1-11; Maestrelli et al. Development of enteric-coated calcium pectinate microspheres intended for colonic drug delivery. Eur J Pharm Biopharm 2008 June; 69(2):508-18; Fude et al. Preparation and in vitro evaluation of pH, time-based and enzyme-degradable pellets for colonic drug delivery. Drug Dev Ind Pharm 2007 September; 33(9):999-1007; Oosegi et al. Novel preparation of enteric-coated chitosan-prednisolone conjugate microspheres and in vitro evaluation of their potential as a colonic delivery system. Eur J Pharm Biopharm, 2008 February; 68(2):260-6; Nunthanid et al. Development of time-, pH-, and enzyme-controlled colonic drug delivery using spray-dried chitosan acetate and hydroxypropyl methylcellulose. Eur J Pharm Biopharm, 2008 February; 68(2):253-9; Singh Characterization and relevance of physicochemical interactions among components of a novel multiparticulate formulation for colonic delivery. Int J Pharm, 2007 Aug. 16; 341 (1-2):143-51; Gazzaniga et al. Oral Colon Delivery: Rationale & Time-based Drug Design Strategy. Discov Med, 2006 December; 6(36):223-8; Singh Modified-release solid formulations for colonic delivery. Recent Pat Drug Deliv Formula, 2007; 1(1):53-63; Akhgari et al. Combination of time-dependent & pH-dependent polymethacrylates as a single coating formulation for colonic delivery of indomethacin pellets. Int J Pharm, 2006 Aug. 31; 320 (1-2):137-42; Ibekwe et al. A comparative in vitro assessment of the drug release performance of pH-responsive polymers for ileo-colonic delivery. Int J. Pharm. 2006 Feb. 3; 308 (1-2):52-60; Al-Saidan et al. In vitro and in vivo evaluation of guar gum-based matrix tablets of rofecoxib for colonic drug delivery. Curr Drug Deliv, 2005 April; 2(2):155-63; Basit. Advances in colonic drug delivery. Drugs. 2005; 65(14):1991-2007; Bott et al. In vivo evaluation of a novel pH- & time-based multiunit colonic drug delivery system. Aliment Pharmacol Ther. 2004 Aug. 1; 20(3):347-53; Qi et al. A novel pH- and time-dependent system for colonic drug delivery. Drug Dev Ind Pharm. 2003 July; 29(6):661-7; 21: Shareef et al. Colonic drug delivery: an updated review. AAPS PharmSci. 2003; 5 (2):E17. Review. PubMed PMID: 12866944; Tuleu et al. Colonic delivery of sodium butyrate via oral route: acrylic coating design of pellets and in vivo evaluation in rats. Methods Find Exp Clin Pharmacol. 2001 June; 23(5):245-53; Gupta et al. A novel pH- & time-based multi-unit potential colonic drug delivery system. II. Optimization of multiple response variables. Int J. Pharm. 2001 Feb. 1; 213 (1-2):93-102; Gupta et al. A novel pH- and time-based multi-unit potential colonic drug delivery system. I. Development. Int J. Pharm. 2001 Feb. 1; 213 (1-2):83-91; Muraoka et al. Evaluation of intestinal pressure-controlled colon delivery capsule containing caffeine as a model drug in human volunteers. J Control Release. 1998 Mar. 2; 52 (1-2):119-29; Niwa et al. Preparation & evaluation of a time-controlled release capsule made of ethylcellulose for colon delivery of drugs. J Drug Target. 1995; 3(2):83-9. Erratum in: J Drug Target 96; 3(6):478; Ashford et al. Targeting drugs to the colon: delivery systems for oral administration. J Drug Target. 1994; 2(3):241-57, all hereby incorporated by reference in their entirety.

In some embodiments of the invention, the coating material may be mixed with various excipients including plasticizers such as triethyl citrate, acetyl triethyl citrate, diethyl phthalate, dibutyl phthalate, dibutyl subacute, dibutyl tartrate, dibutyl maleate, dibutyl succinate and diethyl succinate and inert fillers such as chalk or pigments.

The composition and thickness of the coating may be selected to dissolve immediately upon contact with the digestive juice of the intestine. Alternatively, the composition and thickness of the external coating may be selected to be a time-release coating which dissolves over a selected period of time, as is well known in the art.

The amount of enteric coating depends on the particular coating composition used and is preferably sufficient to substantially prevent the absorption of in the stomach, duodenum, jejunum and, in some embodiments, the ileum as well.

In some embodiments of the invention, hydroxyalkyl celluloses and their aliphatic esters, carboxyalkyl celluloses and their salts, polycarboxymethylene and its salts and derivatives, polyvinyl alcohol and its esters, polycarboxymethylene copolymer with sodium formaldehyde carboxylates, poly-vinylpyrrolidone, and polyethylene glycol and its esters can be applied as coatings by first dissolving the compound in a minimum amount of water. Alcohol is then added to the point of incipient cloudiness. The mixture can then be applied by conventional techniques.

In some embodiments, application of cellulose acetate phthalate may be accomplished by simply dissolving the cellulose acetate phthalate in a minimum amount of alcohol and then applying by conventional techniques. Hydrogenated vegetable oils may be applied by first dissolving the oil in a minimal amount of a non-polymer solvent, such as methylene chloride, chloroform or carbon tetrachloride, then adding alcohol to the point of incipient cloudiness and then applying by conventional techniques.

In some embodiments, the dosage form of is a capsule or tablet, said dosage form pre-coated with an excipient, prior to coating with a polymer.

In some embodiments, the capsule dosage form is sealed after filling in the overlapping region of capsule body and cap by commonly known sealing techniques like banding or applying a sealing liquid and/or heat to the gap between capsule body and cap. Preferred is a sealing process, in which a sealing liquid which may include a solvent applied individually and uniformly to the external edge of the gap of a capsule to be sealed to form a liquid ring around the circumference of the capsule, removing excess sealing liquid from the exterior of the capsule and drying the capsule by applying thermal energy from outside. Such a sealing before coating can prevent problems e.g. with non-uniformity of the coating at the gap or development of fissures during storage under stressing conditions, which can lead to early leaking of the capsule content into the stomach. In some embodiments, the banding is achieved through the application of a ring of liquid gelatin or hypromellose on the external surface of the capsule. In some embodiments, a double-band sealing technique is used to ensure ensures that if an air bubble or unevenness occurs in the first band, it will be eliminated by the second application. Capsule banding can provide tamper evidence, deter counterfeiting, improve mechanical strength, provide color brand differentiation, improve product stability and reduce oxygen diffusion.

For rapid release of the drug from the dosage form in some embodiments, the composition of the coating will usually provide substantial or complete disintegration of the coating in the preferred anatomic location and/or at the preferred time after oral ingestion and/or in the preferred gastrointestinal environment, including preferred pH, preferred osmotic pressure in the lumen, preferred osmotic pressure in the dosage form, preferred hydration level, preferred microbial environment, preferred level of GI peristalsis or agitation.

For controlled or slow release of the drug from the dosage form in some embodiments, the composition of the pH sensitive coating will dissolve, but other controlled release mechanisms will provide for slow release of the drug in the preferred anatomic location and/or at the preferred time after oral ingestion and/or in the preferred gastrointestinal environment, including preferred pH, preferred osmotic pressure in the lumen, preferred osmotic pressure in the dosage form, preferred hydration level, preferred microbial environment, preferred level of GI peristalsis or agitation.

In some embodiments, for rapid release in the small intestine any coating can be used which ensures that the dosage form does not disintegrate until it is emptied from the stomach. The coating may be one which is pH sensitive and which completely dissolves in the small intestine. Typical coating thicknesses will be in the range 2 to 30 mg polymer per cm² of capsule surface, preferably 5 to 15 mg polymer per cm² of capsule surface, but this will vary greatly depending on the choice of coating. For a capsule of size 1 with a surface area of approximately 4 cm² this represents a weight gain of 20 mg to 60 mg per capsule (50 to 150 μm).

In some embodiments, preferred coating materials are those which dissolve at a pH between 5 and 7.6, for example, > about 5, or > about 5.2, or > about 5.5, or > about 5.7, or > about 6, or > about 6.1, or > about 6.2, or > about 6.3, or > about 6.4, or > about 6.5, or > about 6.6, or > about 6.7, or > about 6.8, or > about 6.9, or > about 7, or > about 7.2, or > about 7.4, or > about 7.6.

In some embodiments, preferred coating materials include polymers such as cellulose acetate trimellitiate (CAT), hydroxypropylmethyl cellulose phthalate (HPMCP), polyvinyl acetate phthalate (PVAP), cellulose acetate phthalate (CAP) and shellac. In some embodiments, especially preferred materials for aqueous film coating are copolymers of methacrylic acid and ethyl acrylate, Eudragit® L30D-55 (Roehm GmbH, Darmstadt, Germany).

In some embodiments, for release in the terminal ileum or colon any coating can be used which ensures that the dosage form does not disintegrate until it is reaches the desired location. In some embodiments, the coating may be one which is pH-sensitive, redox-sensitive or sensitive to particular enzymes or bacteria, such that the coating only dissolves or finishes dissolving in the colon. Thus the capsules will not release the drug until it is in the terminal ileum or colon.

In some embodiments, preferred coating materials are those which dissolve at a pH of 7 or above. Generally, such coatings only start to dissolve when they have left the stomach and passed through the duodenum and in cases the jejunum and/or terminal ileum. Generally, by the time the dosage form has reached the terminal ileum or colon the coating will have completely dissolved. Such a coating can be made from a variety of polymers including, without limitation, cellulose acetate trimellitiate (CAT) hydroxypropylmethyl cellulose phthalate (HPMCP), polyvinyl acetate phthalate (PVAP), cellulose acetate phthalate (CAP), shellac and copolymers of methacrylic acid and ethyl acrylate. In some embodiments, especially preferred materials for aqueous film coating are copolymers of methacrylic acid and ethyl acrylate to which a monomer of methylacrylate has been added during polymerization. (Eudragit™ FS 30 D, Roehm GmbH, Darmstadt, Germany). Due to the free carboxylic acid group the polymer dissolves at pH 7 or above making it particularly suitable for delivery into the colon.

It should be noted that when delayed but (subsequently) sustained release of buprenorphine is desired, upon dissolution or disintegration of the pH sensitive coating or material, a variety of mechanisms can provide extended release of the drug, including diffusion from matrix, membranes or pores, osmotic pressure, hydration, etc)

Using preparation Eudragit™ FS 30D a coating thickness of 5 to 15 mg polymer per cm² of capsule surface is preferred in some embodiments.

The colonic region is rich in microbial anaerobic organisms providing reducing conditions. Thus the coating may suitably comprise a material which is redox-sensitive. Such coatings may comprise azopolymers which can for example consist of a random copolymer of styrene and hydroxyethyl methacrylate, cross-linked with divinylazobenzene synthesized by free radical polymerization, the azopolymer being broken down enzymatically and specifically in the colon or may consist of disulphide polymers.

Other materials providing release in the colon are amylose, for example a coating composition can be prepared by mixing amylose-butan-1-ol complex (glassy amylose) with an aqueous dispersion of Ethocel or a coating formulation comprising an inner coating of glassy amylose and an outer coating of cellulose or acrylic polymer material, calcium pectinate, pectin, a polysaccharide which is totally degraded by colonic bacterial enzymes, chondroitin sulfate and resistant starches, dextran hydrogels, modified guar gum such as borax modified guar gum, cyclodextrins, beta.-cyclodextrin, saccharide containing polymers, which can include a polymeric construct comprising a synthetic oligosaccharides-containing biopolymer including methacrylic polymers covalently couples to oligosaccharides such as cellobiose, lactalose, raffinose, and stachyose, or saccharide-containing natural polymers including modified mucopolysaccharides such as cross-linked chondroitin sulfate and metal pectin salts, for example calcium pectate, methacrylate-galactomannan and pH sensitive hydrogels.

Pharmaceutical compositions of the present invention can be prepared using methods described in the art. There is a wide body of literature and other prior art on the delivery, release and absorption of drug from oral dosage forms wherein said delivery, release and absorption is “targeted”, i.e., where said delivery, release and absorption is: (i) achieved at the desired anatomic location of the GI tract; (ii) substantially avoided at certain anatomic locations of the GI tract; (iii) achieved after a particular amount of time has elapsed post-ingestion; (iv) achieved when the GI environment meets certain conditions (e.g., pH, electrolyte concentration, enzymes, hydration, bacterial flora, and the like).

Pharmaceutical compositions of the present invention can be prepared using methods described, referenced or disclosed in U.S. Pat. Nos. 7,196,059, 7,189,414, 7,163,696, 7,157,444, 7,119,079, 7,112,578, 7,109,239, 7,094,425, 7,041,651, 7,030,082, 7,022,683, 6,930,093, 6,919,348, 6,916,791, 6,897,205, 6,893,662, 6,867,183, 6,852,693, 6,824,790, 6,777,000, 6,770,625, 6,761,901, 6,747,014, 6,743,445, 6,734,170, 6,727,287, 6,699,848, 6,692,766, 6,677,321, 6,669,951, 6,632,454, 6,632,451, 6,630,453, 6,555,136, 6,552,072, 6,531,152, 6,525,078, 6,432,967, 6,428,968, 6,346,547, 6,340,476, 6,326,364, 6,277,411, 6,238,689, 6,231,888, 6,228,396, 6,217,904, 6,200,605, 6,200,602, 6,197,763, 6,166,044, 6,166,024, 6,106,864, 6,074,689, 6,063,402, 6,039,975, 5,948,407, 5,914,132, 5,905,081, 5,889,028, 5,866,619, 5,849,327, 5,846,983, 5,843,479, 5,840,332, 5,814,336, 5,811,388, 5,744,166, 5,691,343, 5,686,106, 5,686,105, 5,681,584, 5,672,359, 5,670,158, 5,656,294, 5,656,290, 5,651,983, 5,631,022, 5,554,388, 5,525,634, 5,514,663, 5,482,718, 5,183,802, 5,122,376, 4,904,474, 4,705,515 and 4,627,851, and in US Patent Application No. 20070167416, 20070112075, 20070087939, 20070072828, 20070071820, 20070071806, 20070060580, 20070059366, 20070054945, 20070026067, 20070020254, 20070020197, 20070003626, 20060280795, 20060251720, 20060223787, 20060189635, 20060177507, 20060121091, 20060105045, 20060099243, 20060083718, 20060045865, 20060041109, 20060003995, 20050287276, 20050281781, 20050260262, 20050249716, 20050222040, 20050220861, 20050209271, 20050208132, 20050186267, 20050182134, 20050181053, 20050169996, 20050158408, 20050153908, 20050153907, 20050152978, 20050118326, 20050112201, 20050107334, 20050101611, 20050090473, 20050090451, 20050069550, 20050058701, 20050043298, 20050009848, 20050009768, 20050009767, 20050009766, 20050008702, 20050008688, 20040267240, 20040258754, 20040253304, 20040241173, 20040229831, 20040224898, 20040219216, 20040186045, 20040185107, 20040176319, 20040162263, 20040162259, 20040161459, 20040147445, 20040142880, 20040126422, 20040121967, 20040110837, 20040109894, 20040062778, 20040052846, 20040038866, 20040017387, 20040013687, 20030207851, 20030194439, 20030181380, 20030170181, 20030162717, 20030152617, 20030133978, 20030083232, 20030077326, 20030069170, 20030040497, 20030022843, 20030008914, 20020147156, 20020127198, 20020110593, 20020110590, 20020098235, 20020058061, 20020035139, 20020015729, 20010052137, 20010039262, 20010036473, 20010031748 and 20010026807.

Pharmaceutical compositions of the present invention can be prepared to provide targeted delivery of buprenorphine, wherein the targeted delivery of the immediate or controlled release dosage forms can advantageously provide, among other things, (i) improved efficacy; (ii) improved safety; (iii) reduced appeal to drug addicts, drug abusers and recreational drug users; (iv) reduced nausea; (v) reduced drowsiness; (vi) reduced psychic effects desired by drug addicts, drug abusers and recreational drug users; (vii) reduced desirability for co-abuse with alcohol or other drugs of abuse; and (viii) reduced risk of diversion.

Pharmaceutical compositions of the present invention can be prepared using methods described in the art achieve delivery, release and absorption of drug from oral dosage forms, wherein said delivery, release and absorption is “targeted”, e.g., by way of non-limiting examples, where said delivery, release and absorption is: (i) achieved at the desired anatomic location of the GI tract (e.g., upon arrival in the duodenum, or jejum, or ileum, or ileo-cecal junction, or cecum, or ascending colon, or transverse colon, or descending colon); (ii) substantially avoided at certain anatomic locations of the GI tract (e.g., stomach, or stomach and duodenum, or stomach, duodenum and jejunum, or stomach, duodenum, jejunum and ileum); (iii) achieved after a particular amount of time has elapsed post-ingestion (e.g., ≧1.5 hours or ≧2 hours, or ≧2.5 hours, or ≧3 hours, or ≧3.5 hours, or ≧4 hours, or ≧4.5 hours, or ≧5 hours, or ≧5.5 hours, or ≧6 hours, or ≧6.5 hours, or ≧7 hours, or ≧7.5 hours); (iv) achieved when the dosage form has come in contact or substantial contact or sustained contact with a desired gastrointestinal pH environment (e.g., pH>3, or pH>3.5, or pH>4, or pH >4.5, or pH, >5, or pH>5.5, or pH>6, or pH>7, or pH>7.5, or pH>7.8); (v) achieved when the dosage form has come in contact with desired microbial flora (e.g., colonic microbial flora); (vi) achieved when the GI environment meets certain other conditions (e.g., electrolyte concentration, enzymes, hydration, and the like); (vii) a combination of two or more of the foregoing.

Pharmaceutical compositions of the present invention can be prepared using methods described, referenced or disclosed in U.S. Pat. Nos. 7,196,059, 7,189,414, 7,163,696, 7,157,444, 7,119,079, 7,112,578, 7,109,239, 7,094,425, 7,041,651, 7,030,082, 7,022,683, 6,930,093, 6,919,348, 6,916,791, 6,897,205, 6,893,662, 6,867,183, 6,852,693, 6,824,790, 6,777,000, 6,770,625, 6,761,901, 6,747,014, 6,743,445, 6,734,170, 6,727,287, 6,699,848, 6,692,766, 6,677,321, 6,669,951, 6,632,454, 6,632,451, 6,630,453, 6,555,136, 6,552,072, 6,531,152, 6,525,078, 6,432,967, 6,428,968, 6,346,547, 6,340,476, 6,326,364, 6,277,411, 6,238,689, 6,231,888, 6,228,396, 6,217,904, 6,200,605, 6,200,602, 6,197,763, 6,166,044, 6,166,024, 6,106,864, 6,074,689, 6,063,402, 6,039,975, 5,948,407, 5,914,132, 5,905,081, 5,889,028, 5,866,619, 5,849,327, 5,846,983, 5,843,479, 5,840,332, 5,814,336, 5,811,388, 5,744,166, 5,691,343, 5,686,106, 5,686,105, 5,681,584, 5,672,359, 5,670,158, 5,656,294, 5,656,290, 5,651,983, 5,631,022, 5,554,388, 5,525,634, 5,514,663, 5,482,718, 5,183,802, 5,122,376, 4,904,474, 4,705,515 and 4,627,851, and in US Patent Application No. 20070167416, 20070112075, 20070087939, 20070072828, 20070071820, 20070071806, 20070060580, 20070059366, 20070054945, 20070026067, 20070020254, 20070020197, 20070003626, 20060280795, 20060251720, 20060223787, 20060189635, 20060177507, 20060121091, 20060105045, 20060099243, 20060083718, 20060045865, 20060041109, 20060003995, 20050287276, 20050281781, 20050260262, 20050249716, 20050222040, 20050220861, 20050209271, 20050208132, 20050186267, 20050182134, 20050181053, 20050169996, 20050158408, 20050153908, 20050153907, 20050152978, 20050118326, 20050112201, 20050107334, 20050101611, 20050090473, 20050090451, 20050069550, 20050058701, 20050043298, 20050009848, 20050009768, 20050009767, 20050009766, 20050008702, 20050008688, 20040267240, 20040258754, 20040253304, 20040241173, 20040229831, 20040224898, 20040219216, 20040186045, 20040185107, 20040176319, 20040162263, 20040162259, 20040161459, 20040147445, 20040142880, 20040126422, 20040121967, 20040110837, 20040109894, 20040062778, 20040052846, 20040038866, 20040017387, 20040013687, 20030207851, 20030194439, 20030181380, 20030170181, 20030162717, 20030152617, 20030133978, 20030083232, 20030077326, 20030069170, 20030040497, 20030022843, 20030008914, 20020147156, 20020127198, 20020110593, 20020110590, 20020098235, 20020058061, 20020035139, 20020015729, 20010052137, 20010039262, 20010036473, 20010031748 and 20010026807, which are hereby fully incorporated by reference herein in their entirety.

Pharmaceutical compositions of the present invention can be prepared using methods described, referenced or disclosed in Singh and Kim, Int J. Pharm. 2007; 341:143-51; Jain et al., Crit. Rev Ther Drug Carrier Syst. 2006; 23:349-400; Ugurlu et al., Eur J Pharm Biopharm. 2007; 67:202-10; Sinha et al., J Pharm Pharmacol. 2007; 59:359-65; Gazzaniga et al., Discov Med. 2006; 6:223-8; Rhaman et al., AAPS PharmSciTech. 2006; 7:E47; Akhgari et al., Int J. Pharm. 2006; 320:137-42; Bourgeois et al., J Drug Target. 2005; 13:277-84; Curini et al., Bioorg Med Chem. Lett. 2005; 15:5049-52; Lamprecht et al., J Control Release. 2005; 104:337-46; Verbeke et al., Aliment Pharmacol Ther. 2005; 21:187-94; Bruce et al., Eur J Pharm Biopharm. 2005; 59:85-97; Lamprecht et al., Eur J Pharm Biopharm. 2004; 58:37-43; Mura et al., J Drug Target. 2003; 11:365-71; Lamprecht et al., J Control Release. 2003; 90:313-22; Wiwattanapatapee et al., J Control Release. 2003; 88:1-9; Waterman et al., J Control Release. 2003; 86:293-304; Park et al., Arch Pharm Res. 2002; 25:964-8; Tuleu et al., Aliment Pharmacol Ther. 2002; 16:1771-9; Gupta et al., Drug Dev Ind Pharm. 2002; 28:207-15; Turkoglu et al., Eur J Pharm Biopharm. 2002; 53:65-73; Tuleu et al., Methods Find Exp Clin Pharmacol. 2001; 23:245-53; Stubbe et al., J Control Release. 2001; 75:103-14; Shibata et al., J Pharm Pharmacol. 2001; 53:441-7; Jeong et al., J Control Release. 2001; 71:175-82; Hu et al., Pharm Pharmacol. 2000; 52:1187-93; Hu et al., Pharm Res. 2000; 17:160-7; Ahrabi et al., Eur Pharm Sci. 2000; 10:43-52; Yoshikawa et al., J Pharm Pharmacol. 1999; 51:979-89; Ahrabi et al., Drug Dev Ind Pharm. 1999; 25:453-62; Hu et al., J Control Release. 1998; 56:293-302; Kakoulides et al., J Control Release. 1998; 52:291-300; Kakoulides et al., J Control Release. 1998; 54:95-109; Muraoka et al., J Control Release. 1998; 52:119-29; Takaya et al., J Control Release. 1998; 50:111-22; Muraoka et al., Nippon Rinsho. 1998; 56:788-94; Kenyon et al, Aliment Pharmacol Ther. 1997; 11:205-13; Takaya et al., J Drug Target. 1997; 4:271-6; Matsuda et al., J Drug Target. 1996; 4:59-67; Jones S P, J Drug Target. 1996; 3:477-8; Fedorak et al., Gastroenterology. 1995; 108:1688-99; Takaya et al., J Pharm Pharmacol. 1995; 47:474-8; Niwa et al., J Drug Target. 1995; 3:83-9; Ashford and Fell, J Drug Target. 1994; 2:241-57, Colonic Drug Delivery (page 287-294), Wilson C G, In: Modified-Release Drug Delivery Technology, Second Edition, Vol. 1, Rathbone M J, Hadgraft J, Roberts M S, Lane M E (eds), Informa Heakthcare USA Inc. 2008; Biopolymers and Colonic Delivery, Wilson C G, Mukherji G, Shah H K (pages 295-309), In: Modified-Release Drug Delivery Technology, Second Edition, Vol. 1, Rathbone M J, Hadgraft J, Roberts M S, Lane M E (eds), Informa Heakthcare USA Inc. 2008; Enteric Coating for Colonic Delivery, Shah H K, Mukherji G, Brogmann B, Wilson C G (pages 311-324), In: Modified-Release Drug Delivery Technology, Second Edition, Vol. 1, Rathbone M J, Hadgraft J, Roberts M S, Lane M E (eds), Informa Heakthcare USA Inc. 2008; Programmed Drug Delivery Systems and the Colon, Wilson C G, Shah H K, Lee W W, Brogmann B, Mukherji G (pages 325-335), In: Modified-Release Drug Delivery Technology, Second Edition, Vol. 1, Rathbone M J, Hadgraft J, Roberts M S, Lane M E (eds), Informa Heakthcare USA Inc. 2008; Targeting the Colon Using COLAL™: A Novel Bacteria-Sensitive Drug Delivery System, McConnell E L, Basit A W (pages 343-348), In: Modified-Release Drug Delivery Technology, Second Edition, Vol. 1, Rathbone M J, Hadgraft J, Roberts M S, Lane M E (eds), Informa Heakthcare USA Inc. 2008, Niazi, S. Handbook of Pharmaceutical Manufacturing Formulations: Compressed Solid Products (Volume 1 of 6), CRC Press, 2004, which are hereby fully incorporated by reference herein in their entirety.

### Oral Immediate Release Dosage Forms

Pharmaceutical composition and methods of the present invention contain buprenorphine base or pharmaceutically acceptable salts in racemic or enantiomeric form, or mixtures thereof in and they are intended for oral administration.

All oral immediate release pharmaceutical dosage forms of the invention are contemplated. The preparation of oral immediate release dosage forms has been described in the art—see Remington: the science of Pharmacy Practice, 21^{st} Edition, 2006, Lippincott, Williams & Wilkins, Baltimore, Md.; Pharmaceutical Preformulation and Formulation: A Practical Guide from Candidate Drug Selection to Commercial Dosage Form. Gibson, M (ed). CRC Press, 2001; Niazi, S. Handbook of Pharmaceutical Manufacturing Formulations: Uncompressed Solid Products (Volume 2 of 6), CRC Press, 2004; Niazi, S. Handbook of Pharmaceutical Manufacturing Formulations: Compressed Solid Products (Volume 1 of 6), CRC Press, 2004; Mollet, H, Grubenmann A, Payne H. Formulation Technology: Emulsions, Suspensions, Solid Forms, Wiley-VCH, 2001; Niazi S and Niazi SK (all of which are hereby incorporated by reference). A majority of oral dosage forms commercially available world-wide are formulated as immediate release products.

### Controlled-Release Dosage Forms

All oral extended release pharmaceutical dosage forms of the invention are contemplated. The preparation of oral extended release pharmaceutical dosage forms has been described in the art—see—see for example, (i) Remington: the science of Pharmacy Practice, 21^{st} Edition, 2006, Lippincott, Williams & Wilkins, Baltimore, Md.; and (ii) Pharmaceutical Preformulation and Formulation: A Practical Guide from Candidate Drug Selection to Commercial Dosage Form. Gibson, M (ed). CRC Press, 2001; and (iii) Niazi, S. Handbook of Pharmaceutical Manufacturing Formulations: Compressed Solid Products (Volume 1 of 6), CRC Press, 2004; and (iv) Mollet, H, Grubenmann A, Payne H. Formulation Technology: Emulsions, Suspensions, Solid Forms, Wiley-VCH, 2001; (v) Donald Wise, Handbook of Pharmaceutical Controlled Release Technology, CRC; 1st edition (Aug. 15, 2000); (vi) Cherng-ju Kim, Controlled Release Dosage Form Design, Informa Healthcare (Oct. 25, 1999); (vii) Xiaoling Li, Design of Controlled Release Drug Delivery Systems, McGraw-Hill Professional; 1 edition (Nov. 3, 2005); (viii) Jean-Maurice Vergnaud, Controlled Drug Release Of Oral Dosage Forms, CRC (Jul. 31, 1993); (ix) L. T. Fan and S. K. Singh. Controlled Release: A Quantitative Treatment, Springer-Verlag (July 1989); (x) Tapash K. Ghosh and Bhaskara R. Jasti (eds). Theory and Practice of Contemporary Pharmaceutics, CRC; 2Rev Ed edition (Nov. 23, 2004); (xi) Xiaoling Li and Bhaskara R. Jasti (eds). Design of Controlled release Drug Delivery Systems; (xii) Anya M. Hillery, Andrew W. Lloyd and James Swarbrick (eds). Drug Delivery and Targeting: For Pharmacists and Pharmaceutical Scientists, CRC (Sep. 27, 2001); (xiii) Ram I. Mahato. Pharmaceutical Dosage Forms and Drug Delivery, CRC; 1 edition (Jun. 7, 2007); (xiv) Vasant V. Ranade and Mannfred A. Hollinger. Drug Delivery Systems, Second Edition, CRC; 2 edition (Aug. 26, 2003); (xv) Ashok Katdare and Mahesh Chaubal (eds). Excipient Development for Pharmaceutical, Biotechnology, and Drug Delivery Systems, Informa Healthcare; 1 edition (Jul. 28, 2006); and (xvi) Binghe Wang, Teruna J. Siahaan and Richard A. Soltero. Drug Delivery: Principles and Applications, Wiley-Interscience (Mar. 28, 2005), all of which are hereby incorporated in their entirety by reference.

A wide variety of methods for the preparation of controlled release dosage form are known in the art. These methods may be employed for the preparation of controlled release dosage forms of the invention, including but not limited to: (i) Diffusion-controlled Products: in some embodiments such products employ a water-insoluble polymer to control the flow of water and the subsequent egress of dissolved drug from the dosage form. Both diffusion and dissolution processes are involved. In “reservoir” systems, a core of drug is coated with the polymer and, in “matrix” systems, the drug is dispensed throughout the matrix. Cellulose derivatives are commonly used in the reservoir systems, while the matrix systems may use methylacrylate-methyl methacrylate, polyvinyl chloride, hydrophilic polymers such as cellulose derivatives or fatty compounds including carnauba wax; (ii) Dissolution-Controlled Products: in some embodiments such products control the rate of dissolution of the drug (and therefore absorption) by slowly soluble polymers or by microencapsulation. Once the coating is dissolved, the active drug becomes available for dissolution. By varying the thicknesses or amount of coating and its composition, the rate of active drug release can be controlled. Some dosage forms contain a portion of the total dose as in immediate release form to provide an early “pulse dose”. Spheroid (pellet) dosage forms of diffusion or dissolution-controlled products can be encapsulated or prepared as a tablet. One potential advantage of encapsulated spheroid products is that the onset of absorption is less sensitive to gastric emptying, since the entry of the spheroids into the duodenum tends to be more uniform than with non-disintegrating extended-release tablet formulations; (iii) Erosion Products: in some embodiments, such products control the release of active drug by the erosion rate of a carrier matrix. The release rate is determined by the rate of erosion; (iv) Osmotic Pump Systems: in some embodiments such products control the rate of release of active drug by the constant inflow of water across a semipermeable membrane into a reservoir which contains an osmotic agent. The drug is either mixed with the agent or is located in a reservoir. The dosage form contains one or more small passageways, or through pores within a membrane through which drug in suspension or solution is pumped at a rate determined by the rate of entry of water due through osmotic pressure. The rate of release is can be kept relatively constant; (v) Ion Exchange Resins: in some embodiments, such products control the release of active drug bound to an ion exchange resin by release of drug in the ionic environment within the GI tract.

The preparation of oral extended release pharmaceutical dosage forms has also been described in the art. Nonlimiting examples are provided in U.S. Pat. Nos. 7,427,414; 7,422,758; 7,413,750; 7,413,749; 7,387,793; 7,316,821; 7,229,642; 7,198,803; 7,189,414; 7,125,567; 7,074,430; 7,070,806; 7,052,706; 6,979,463; 6,936,275; 6,932,981; 6,905,709; 6902742; 6793936; 6733783; 6730325; 6726931; 6716449; 6709677; 6699508; 6699506; 6692769; 6692766; 6682759; 6667060; 6645527; 6599529; 6579536; 6517868; 6440458; 6387404; 6344215; 6342250; 6326027; 6319520; 6306438; 6274599; 6254887; 6245356; 6245351; 6228398; 6221399; 6210714; 6162463; 6159501; 6156342; 6153623; 6143353; 6143322; 6132772; 6103261; 6074674; 6048548; 6039980; 6034085; 6030642; 6024982; 5952005; 5885616; 5869100; 5858408; 5795882; 5773025; 5681585; 5674533; 5656295; 5656291; 5639476; 5614218; 5591452; 5589190; 5582837; 5580578; 5549912; 5520931; 5512293; 5508042; 5500227; 5484607; 5472712; 5451409; 5399358; 5378474; 5334392; 5330766; 5314697; 5281415; 5262164; 5242910; 5229135; 5202128; 5198220; 5196202; 5186930; 5173299; 5128144; 5114718; 5084267; 5077051; 5047248; and 5043165, and in U.S. Patent application No. 20090060994; 20090017127; 20090017126; 20080318910; 20080312309; 20080305160; 20080299196; 20080299189; 20080274181; 20080274177; 20080268057; 20080234352; 20080226713; 20080221174; 20080206335; 20080138411; 20080124399; 20080124398; 20080118556; 20080113025; 20080102121; 20080095853; 20080075785; 20080075781; 20080070972; 20080069888; 20080069873; 20080063711; 20080057123; 20080026052; 20080020039; 20080003281; 20070298101; 20070275065; 20070275062; 20070264325; 20070219175; 20070207214; 20070196500; 20070196396; 20070185218; 20070166375; 20070160663; 20070149590; 20070148153; 20070128276; 20070122481; 20070077297; 20070071819; 20070048377; 20070003617; 20060269603; 20060269601; 20060251721; 20060240105; 20060233880; 20060228413; 20060210631; 20060210630; 20060204578; 20060193912; 20060193911; 20060165808; 20060165807; 20060165792; 20060165791; 20060147527; 20060128806; 20060110463; 20060099262; 20060099261; 20060073204; 20060068009; 20060057203; 20060024366; 20050244498; 20050106247; 20040213844; 20040197405; 20040132826; 20040122104; 20040076665; 20040052851; 20030170304; 20030129237 and 20020054907, all of which are hereby incorporated in their entirety by reference.

Matrix-Based Dosage Forms

In some embodiments, the modified release formulations of the present invention are provided as matrix-based dosage forms. Matrix formulations according to the invention may include hydrophilic, e.g., water-soluble, and/or hydrophobic, e.g., water-insoluble, polymers. The matrix formulations of the present invention may optionally be prepared with functional coatings, which may be enteric, e.g., exhibiting a pH-dependent solubility, or non-enteric, e.g., exhibiting a pH-independent solubility.

Matrix formulations of the present invention may be prepared by using, for example, direct compression or wet granulation. A functional coating, as noted above, may then be applied in accordance with the invention. Additionally, a barrier or sealant coat may be applied over a matrix tablet core prior to application of a functional coating. The barrier or sealant coat may serve the purpose of separating an active ingredient from a functional coating, which may interact with the active ingredient, or it may prevent moisture from contacting the active ingredient.

In a matrix-based dosage form in accordance with the present invention, the buprenorphine and optional pharmaceutically acceptable excipient(s) are dispersed within a polymeric matrix, which typically comprises one or more water-soluble polymers and/or one or more water-insoluble polymers. The drug may be released from the dosage form by diffusion and/or erosion.

In one embodiment, a matrix-based dosage form comprises buprenorphine, a filler such as starch, lactose, or microcrystalline cellulose; a controlled-release polymer, such as hydroxypropyl methylcellulose or polyvinyl pyrrolidone; a disintegrant, such crospovidone, or starch; a lubricant, such as magnesium stearate or stearic acid; a surfactant, such as sodium lauryl sulfate or polysorbates; and a glidant, such as colloidal silicon dioxide or talc. The amounts and types of polymers, and the ratio of water-soluble polymers to water-insoluble polymers in the inventive formulations are generally selected to achieve a desired release profile of buprenorphine. For example, by increasing the amount of water insoluble-polymer relative to the amount of water soluble-polymer, the release of the drug may, in some embodiments, be delayed or slowed. This is due, in part, to an increased impermeability of the polymeric matrix, and, in some cases, to a decreased rate of erosion during transit through the GI tract.

The controlled-release dosage form may optionally include a controlled release material which is incorporated into a matrix along with the buprenorphine, or which is applied as a sustained release coating over a substrate comprising the drug (the term “substrate” encompassing beads, pellets, spheroids, tablets, tablet cores, etc). The controlled release material may be hydrophobic or hydrophilic as desired. The oral dosage form according to the invention may be provided as, for example, granules, spheroids, pellets or other multiparticulate formulations. An amount of the multiparticulates which is effective to provide the desired dose of buprenorphine over time may be placed in a capsule or may be incorporated in any other suitable oral solid form, e.g., compressed into a tablet. On the other hand, the oral dosage form according to the present invention may be prepared as a tablet core coated with a controlled-release coating, or as a tablet comprising a matrix of drug and controlled release material, and optionally other pharmaceutically desirable ingredients (e.g., diluents, binders, colorants, lubricants, etc.). The controlled release dosage form of the present invention may also be prepared as a bead formulation or an osmotic dosage formulation.

In certain preferred embodiments of the present invention, the controlled-release formulation is achieved via a matrix (e.g. a matrix tablet) which includes a controlled-release material as set forth below. A dosage form including a controlled-release matrix provides in-vitro dissolution rates of buprenorphine within the preferred ranges and that releases the buprenorphine in a pH-dependent or pH-independent manner. The materials suitable for inclusion in a controlled-release matrix will depend on the method used to form the matrix. The oral dosage form may contain between 1% and 99% (by weight) of at least one hydrophilic or hydrophobic controlled release material.

A non-limiting list of suitable controlled-release materials which may be included in a controlled-release matrix according to the invention include hydrophilic and/or hydrophobic materials, such as gums, cellulose ethers, acrylic resins, protein derived materials, waxes, shellac, and oils such as hydrogenated castor oil, hydrogenated vegetable oil hydrogenated Type I or Type II vegetable oils, polyoxyethylene stearates and distearates, glycerol monostearate, and non-polymeric, non-water soluble liquids carbohydrate-based substances or poorly water soluble, high melting point (mp=40 to 100° C.) waxes and mixtures thereof.

Hydrogenated vegetable oils of the present invention may include hydrogenated cottonseed oil (e.g., Akofine®; Lubritab®; Sterotex® NF), hydrogenated palm oil (Dynasan® P60; Softisan® 154), hydrogenated soybean oil (Hydrocote®; Lipovol HS-K°; Sterotex® HM) and hydrogenated palm kernel oil (e.g., Hydrokote® 112).

Polyoxyethylene stearates and distearates of the present invention include Polyoxyl 2, 4, 6, 8, 12, 20, 30, 40, 50, 100 and 150 stearates (e.g., Hodag® DGS; PEG-2 stearate; Acconon® 200-MS; Hodag® 20-S; PEG-4 stearate; Cerasynt® 616; Kessco® PEG 300 Monostearate; Acconon® 400-MS; Cerasynt® 660; Cithrol® 4MS; Hodag® 60-S; Kessco® PEG 600 Monostearate; Cerasynt® 840; Hodag 100-S; Myrj® 51; PEG-30 stearate; polyoxyethylene (30) stearate; Crodet® S40; E431; Emerest® 2672; Atlas G-2153; Crodet® S50) and polyoxyl 4, 8, 12, 32 and 150 distearates (e.g, Lipo-PEG® 100-S; Myrj® 59; Hodag® 600-S; Ritox® 59; Hodag® 22-S; PEG-4 distearate; Hodag® 42-S; Kessco® PEG 400 DS; Hodag® 62-S; Kessco® PEG 600 Distearate; Hodag® 154-S; Kessco® PEG 1540 Distearate; Lipo-PEG® 6000-DS; Protamate® 6000-DS).

In one embodiment of the present invention, the buprenorphine is combined with beeswax, hydroxypropyl methyl cellulose (e.g, HPMC K15M), silicon dioxide (alone or in combination with Al₂O₃; e.g, Aerosil®, Aerosil® 200, Aerosil® COK84).

In one embodiment of the present invention, the buprenorphine is combined with hydrogenated cottonseed oil (e.g., Sterotex® NF), hydroxypropyl methyl cellulose (e.g, HPMC K15M), coconut oil and silicon dioxide (alone or in combination with Al₂O₃; e.g, Aerosil®, Aerosil® 200, Aerosil® COK84).

In another embodiment of the present invention, the buprenorphine is combined with glycerol monostearate (e.g., Cithrol® GMS), hydroxypropyl methyl cellulose (e.g, HPMC K100M) and silicon dioxide (alone or in combination with Al₂O₃; e.g, Aerosil®, Aerosil® 200, Aerosil® COK84).

In yet another embodiment of the present invention, the buprenorphine is combined with hydrogenated palm kernel oil (e.g., Hydrokote® 112), hydroxypropyl methyl cellulose (e.g, HPMC K15M) and silicon dioxide (alone or in combination with Al₂O₃; e.g, Aerosil®, Aerosil® 200, Aerosil® COK84).

In one embodiment of the present invention, release rate modifiers, including hydroxypropyl methyl cellulose (e.g, HPMC K15M) may incorporated. Release rate modifiers can also have additional useful properties that optimize the formulation.

A variety of agents may be incorporated into the invention as thixotropes (e.g., fumed silicon dioxides, Aerosil®, Aerosil® COK84, Aerosil® 200, etc.). Thixotropes enhance the pharmaceutical formulations of the invention by increasing the viscosity of solutions complementing the action of HPMCs.

Any pharmaceutically acceptable hydrophobic or hydrophilic controlled-release material which is capable of imparting controlled-release of the buprenorphine may be used in accordance with the present invention. Preferred controlled-release polymers include alkylcelluloses such as ethylcellulose, acrylic and methacrylic acid polymers and copolymers, and cellulose ethers, especially hydroxyalkylcelluloses (e.g., hydroxypropylmethylcellulose) and carboxyalkylcelluloses. Preferred acrylic and methacrylic acid polymers and copolymers include methyl methacrylate, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamine copolymer, poly(methyl methacrylate), poly(methacrylic acid) (anhydride), polymethacrylate, polyacrylamide, poly(methacrylic acid anhydride), glycidyl methacrylate copolymers, ethylcellulose, cellulose acetate cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly (ethylene terephthalate), poly(vinyl isobutyl ether), poly(vinyl acetate), poly(vinyl chloride) or polyurethane, and/or mixtures thereof. Certain preferred embodiments utilize mixtures of any of the foregoing controlled-release materials in the matrices of the invention.

The matrix also may include a binder. In such embodiments, the binder preferably contributes to the controlled-release of the buprenorphine from the controlled-release matrix.

Preferred hydrophobic binder materials are water-insoluble with more or less pronounced hydrophilic and/or hydrophobic trends. Preferred hydrophobic binder materials which may be used in accordance with the present invention include digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils, natural and synthetic waxes and polyalkylene glycols. Preferably, the hydrophobic binder materials useful in the invention have a melting point from about 30 to about 200° C., preferably from about 45 to about 90° C. When the hydrophobic material is a hydrocarbon, the hydrocarbon preferably has a melting point of between 25 and 90° C. Of the long chain (C₈-C₅₀) hydrocarbon materials, fatty (aliphatic) alcohols are preferred. The oral dosage form may contain up to 98% (by weight) of at least one digestible, long chain hydrocarbon.

The oral dosage form contains up to 98% (by weight) of at least one polyalkylene glycol. The hydrophobic binder material may comprise natural or synthetic waxes, fatty alcohols (such as lauryl, myristyl, stearyl, cetyl or preferably cetostearyl alcohol), fatty acids, including but not limited to fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones. Suitable waxes include, for example, beeswax, glycowax, castor wax and carnauba wax. For purposes of the present invention, a wax-like substance is defined as any material which is normally solid at room temperature and has a melting point of from about 30 to about 100° C.

In certain preferred embodiments, a combination of two or more hydrophobic binder materials are included in the matrix formulations. If an additional hydrophobic binder material is included, it is preferably selected from natural and synthetic waxes, fatty acids, fatty alcohols, and mixtures of the same. Examples include beeswax, carnauba wax, stearic acid and stearyl alcohol. This list is not meant to be exclusive.

One particular suitable controlled-release matrix comprises at least one water soluble hydroxyalkyl cellulose, at least one C₁₂-C₃₆, preferably C₁₄-C₂₂, aliphatic alcohol and, optionally, at least one polyalkylene glycol. The hydroxyalkyl cellulose is preferably a hydroxy (C₁ to C₆) alkyl cellulose, such as hydroxypropylcellulose, hydroxypropylmethylcellulose and, especially, hydroxyethyl cellulose. The amount of the at least one hydroxyalkyl cellulose in the present oral dosage form will be determined, inter alia, by the precise rate of the buprenorphine release required. The aliphatic alcohol may be, for example, lauryl alcohol, myristyl alcohol or stearyl alcohol. In particularly preferred embodiments of the present oral dosage form, however, the at least one aliphatic alcohol is cetyl alcohol or cetostearyl alcohol. The amount of aliphatic alcohol in the present oral dosage form will be determined, as above, by the precise rate of the buprenorphine release required. It will also depend on whether at least one polyalkylene glycol is present in or absent from the oral dosage form. In the absence of at least one polyalkylene glycol, the oral dosage form preferably contains between 20% and 50% (by wt) of the aliphatic alcohol. When a polyalkylene glycol is present in the oral dosage form, then the combined weight of the aliphatic alcohol and the polyalkylene glycol preferably constitutes between 20% and 50% (by wt) of the total dosage.

In one preferred embodiment, the ratio of, e.g., the at least one hydroxyalkyl cellulose or acrylic resin to the at least one aliphatic alcohol/polyalkylene glycol determines, to a considerable extent, the release rate of the buprenorphine from the formulation. A ratio of the hydroxyalkyl cellulose to the aliphatic alcohol/polyalkylene glycol of between 1:2 and 1:4 is preferred, with a ratio of between 1:3 and 1:4 being particularly preferred.

The polyalkylene glycol maybe, for example, polypropylene glycol or, which is preferred, polyethylene glycol. The number average molecular weight of the at least one polyalkylene glycol is preferred between 1,000 and 15,000 especially between 1,500 and 12,000.

Another suitable controlled-release matrix comprises an alkylcellulose (especially ethylcellulose), a C₁₂ to C₃₆ aliphatic alcohol and, optionally, a polyalkylene glycol.

Another method of producing the dosage form of the invention involves liquid fill compositions, including hydrogenated Type I or Type II vegetable oils (e.g., Hydrokote® 112), polyoxyethylene stearates and distearates, glycerol monostearate (e.g., Cithrol® GMS), non-polymeric, non-water soluble liquids carbohydrate-based substances, poorly water soluble, high melting point (mp=40 to 100° C.) waxes.

Hydrogenated vegetable oils may include hydrogenated cottonseed oil (e.g., Akofine®; Lubritab®; Sterotex® NF), hydrogenated palm oil (Dynasan® P60; Softisan® 154), hydrogenated soybean oil (Hydrocote®; Lipovol HS-K®; Sterotex® HM) and hydrogenated palm kernel oil (e.g., Hydrokote® 112).

Polyoxyethylene stearates and distearates may include Polyoxyl 2, 4, 6, 8, 12, 20, 30, 40, 50, 100 and 150 stearates (e.g., Hodag® DGS; PEG-2 stearate; Acconon® 200-MS; Hodag® 20-S; PEG-4 stearate; Cerasynt® 616; Kessco® PEG 300 Monostearate; Acconon® 400-MS; Cerasynt® 660; Cithrol® 4MS; Hodag® 60-S; Kessco® PEG 600 Monostearate; Cerasynt® 840; Hodag 100-S; Myrj® 51; PEG-30 stearate; polyoxyethylene (30) stearate; Crodet® S40; E431; Emerest® 2672; Atlas G-2153; Crodet® S50) and polyoxyl 4, 8, 12, 32 and 150 distearates (e.g, Lipo-PEG® 100-S; Myrj® 59; Hodag® 600-S; Ritox® 59; Hodag® 22-S; PEG-4 distearate; Hodag® 42-S; Kessco® PEG 400 DS; Hodag® 62-S; Kessco® PEG 600 Distearate; Hodag® 154-S; Kessco® PEG 1540 Distearate; Lipo-PEG® 6000-DS; Protamate® 6000-DS).

In one embodiment of the present invention, release rate modifiers, including hydroxypropyl methyl cellulose (e.g, HPMC K15M) may be incorporated. Release rate modifiers can also have additional useful properties that optimize the formulation.

A variety of agents may be incorporated into the invention as thixotropes (e.g., fumed silicon dioxides, Aerosil®, Aerosil® COK84, Aerosil® 200, etc.). Thixotropes enhance the pharmaceutical formulations of the invention by increasing the viscosity of solutions during attempted extraction, complementing the action of HPMCs. They may also provide a tamper resistance by helping to retain the structure of dosage units that have been heated to temperatures greater than the melting point of the base excipient (Aerosils are unaffected by heat).

In addition to the above ingredients, a controlled-release matrix may also contain suitable quantities of other materials, e.g., diluents, lubricants, binders, granulating aids, colorants, flavorants and glidants that are conventional in the pharmaceutical art.

In order to facilitate the preparation of a solid, controlled-release oral dosage form according to the invention there is provided, in a further aspect of the present invention, a process for the preparation of a solid, controlled-release oral dosage form according to the present invention comprising incorporating the buprenorphine or a salt thereof in a controlled-release matrix. Incorporation in the matrix may be effected, for example, by (a) forming granules comprising at least one hydrophobic and/or hydrophilic material as set forth above (e.g., a water soluble hydroxyalkyl cellulose) together with the buprenorphine; (b) mixing the at least one hydrophobic and/or hydrophilic material-containing granules with at least one C₁₂-C₃₆ aliphatic alcohol, and (c) optionally, compressing and shaping the granules.

The granules may be formed by any of the procedures well-known to those skilled in the art of pharmaceutical formulation. For example, in one preferred method, the granules may be formed by wet granulating hydroxyalkyl cellulose/buprenorphine with water. In a particular preferred embodiment of this process, the amount of water added during the wet granulation step is preferably between 1.5 and 5 times, especially between 1.75 and 3.5 times, the dry weight of the buprenorphine.

In certain embodiments, the dosage form comprises a plurality of matrices described above.

The matrices of the present invention may also be prepared via a melt pellitization technique. In such circumstance, the buprenorphine in finely divided form is combined with a binder (also in particulate form) and other optional inert ingredients, and thereafter the mixture is pelletized, e.g., by mechanically working the mixture in a high shear mixer to form the pellets (granules, spheres). Thereafter, the pellets (granules, spheres) may be sieved in order to obtain pellets of the requisite size. The binder material is preferably in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like.

Controlled-release matrices can also be prepared by, e.g., melt-granulation or melt-extrusion techniques. Generally, melt-granulation techniques involve melting a normally solid hydrophobic binder material, e.g. a wax, and incorporating a powdered drug therein. To obtain a controlled release dosage form, it may be necessary to incorporate a hydrophobic controlled release material, e.g. ethylcellulose or a water-insoluble acrylic polymer, into the molten wax hydrophobic binder material.

The hydrophobic binder material may comprise one or more water-insoluble wax-like thermoplastic substances possibly mixed with one or more wax-like thermoplastic substances being less hydrophobic than said one or more water-insoluble wax-like substances. In order to achieve controlled release, the individual wax-like substances in the formulation should be substantially non-degradable and insoluble in gastrointestinal fluids during the initial release phases. Useful water-insoluble wax-like binder substances may be those with a water-solubility that is lower than about 1:5,000 (w/w).

In addition to the above ingredients, a controlled release matrix may also contain suitable quantities of other materials, e.g., diluents, lubricants, binders, granulating aids, colorants, flavorants and glidants that are conventional in the pharmaceutical art in amounts up to about 50% by weight of the particulate if desired. The quantities of these additional materials will be sufficient to provide the desired effect to the desired formulation.

The preparation of a suitable melt-extruded matrix according to the present invention may, for example, include the steps of blending the buprenorphine, together with a controlled release material and preferably a binder material to obtain a homogeneous mixture. The homogeneous mixture is then heated to a temperature sufficient to at least soften the mixture sufficiently to extrude the same. The resulting homogeneous mixture is then extruded, e.g., using a twin-screw extruder, to form strands. The extrudate is preferably cooled and cut into multiparticulates by any means known in the art. The strands are cooled and cut into multiparticulates. The multiparticulates are then divided into unit doses. The extrudate preferably has a diameter of from about 0.1 to about 5 mm and provides controlled release of the therapeutically active agent for a time period of from about 6 to at least about 24 hours.

An optional process for preparing the melt extrusioned formulations of the present invention includes directly metering into an extruder a hydrophobic controlled release material, a therapeutically active agent, and an optional binder material; heating the homogenous mixture; extruding the homogenous mixture to thereby form strands; cooling the strands containing the homogeneous mixture; cutting the strands into particles having a size from about 0.1 mm to about 12 mm; and dividing said particles into unit doses. In this aspect of the invention, a relatively continuous manufacturing procedure is realized.

Plasticizers, such as those described herein, may be included in melt-extruded matrices. The plasticizer is preferably included as from about 0.1 to about 30% by weight of the matrix. Other pharmaceutical excipients, e.g., talc, mono or poly saccharides, colorants, flavorants, lubricants and the like may be included in the controlled release matrices of the present invention as desired. The amounts included will depend upon the desired characteristic to be achieved.

The diameter of the extruder aperture or exit port can be adjusted to vary the thickness of the extruded strands. Furthermore, the exit part of the extruder need not be round; it can be oblong, rectangular, etc. The exiting strands can be reduced to particles using a hot wire cutter, guillotine, etc.

A melt extruded multiparticulate system can be, for example, in the form of granules, spheroids or pellets depending upon the extruder exit orifice. For purposes of the present invention, the terms “melt-extruded multiparticulate(s)” and “melt-extruded multiparticulate system(s)” and “melt-extruded particles” shall refer to a plurality of units, preferably within a range of similar size and/or shape and containing one or more active agents and one or more excipients, preferably including a hydrophobic controlled release material as described herein. Preferably the melt-extruded multiparticulates will be of a range of from about 0.1 to about 12 mm in length and have a diameter of from about 0.1 to about 5 mm. In addition, it is to be understood that the melt-extruded multiparticulates can be any geometrical shape within this size range. Alternatively, the extrudate may simply be cut into desired lengths and divided into unit doses of the therapeutically active agent without the need of a spheronization step.

In one preferred embodiment, oral dosage forms are prepared that include an effective amount of melt-extruded multiparticulates within a capsule. For example, a plurality of the melt-extruded multiparticulates may be placed in a gelatin capsule in an amount sufficient to provide an effective controlled release dose when ingested and contacted by gastric fluid.

In another preferred embodiment, a suitable amount of the multiparticulate extrudate is compressed into an oral tablet using conventional tableting equipment using standard techniques. Techniques and compositions for making tablets (compressed and molded), capsules (hard and soft gelatin) and pills are also described in Remington's Pharmaceutical Sciences, 21^{st} ed., 2005 incorporated by reference herein.

In yet another preferred embodiment, the extrudate can be shaped into tablets as set forth in U.S. Pat. No. 4,957,681, hereby incorporated by reference.

Optionally, the controlled-release matrix multiparticulate systems or tablets can be coated, or the gelatin capsule can be further coated, with a controlled release coating such as the controlled release coatings described above. Such coatings preferably include a sufficient amount of hydrophobic and/or hydrophilic controlled-release material to obtain a weight gain level from about 2 to about 25 percent, although the overcoat may be greater depending upon, e.g., the physical properties of the drug and the desired release rate, among other things.

The dosage forms of the present invention may further include combinations of melt-extruded multiparticulates containing one or more drugs. Furthermore, the dosage forms can also include an amount of an immediate release therapeutically active agent for prompt therapeutic effect. The immediate release therapeutically active agent may be incorporated, e.g., as separate pellets within a gelatin capsule, or may be coated on the surface of, e.g., melt extruded multiparticulates. The unit dosage forms of the present invention may also contain a combination of, e.g., controlled release beads and matrix multiparticulates to achieve a desired effect.

The controlled-release formulations of the present invention preferably slowly release the therapeutically active agent, e.g., when ingested and exposed to gastric fluids, and then to intestinal fluids. The controlled-release profile of the melt-extruded formulations of the invention can be altered, for example, by varying the amount of controlled-release material, by varying the amount of plasticizer relative to other matrix constituents, hydrophobic material, by the inclusion of additional ingredients or excipients, by altering the method of manufacture, etc.

In other embodiments of the invention, melt-extruded formulations are prepared without the inclusion of the therapeutically active agent, which is added thereafter to the extrudate. Such formulations typically will have the therapeutically active agent blended together with the extruded matrix material, and then the mixture would be tableted in order to provide a slow release formulation. Such formulations may be advantageous, for example, when the therapeutically active agent included in the formulation is sensitive to temperatures needed for softening the hydrophobic material and/or the retardant material.

Typical melt-extrusion production systems suitable for use in accordance with the present invention include a suitable extruder drive motor having variable speed and constant torque control, start-stop controls, and ammeter. In addition, the production system will include a temperature control console which includes temperature sensors, cooling means and temperature indicators throughout the length of the extruder. In addition, the production system will include an extruder such as twin-screw extruder which consists of two counter-rotating intermeshing screws enclosed within a cylinder or barrel having an aperture or die at the exit thereof. The feed materials enter through a feed hopper and are moved through the barrel by the screws and are forced through the die into strands which are thereafter conveyed such as by a continuous movable belt to allow for cooling and being directed to a pelletizer or other suitable device to render the extruded ropes into the multiparticulate system. The pelletizer can consist of rollers, fixed knife, rotating cutter and the like. Suitable instruments and systems will be apparent to those of ordinary skill in the art.

A further aspect of the invention is related to the preparation of melt-extruded multiparticulates as set forth above in a manner which controls the amount of air included in the extruded product. By controlling the amount of air included in the extrudate, the release rate of the therapeutically active agent from the, e.g., multiparticulate extrudate, can be altered significantly. In certain embodiments, the pH dependency of the extruded product can be altered as well.

Thus, in a further aspect of the invention, the melt-extruded product is prepared in a manner which substantially excludes air during the extrusion phase of the process. This may be accomplished, for example, by using a Leistritz extruder having a vacuum attachment. In certain preferred embodiments the extruded multiparticulates prepared according to the invention using the Leistritz extruder under vacuum provides a melt-extruded product having different physical characteristics. In particular, the extrudate is substantially non-porous when magnified, e.g., using a scanning electron microscope which provides an SEM (scanning electron micrograph). Such substantially non-porous formulations provide a faster release of the therapeutically active agent, relative to the same formulation prepared without vacuum. SEMs of the multiparticulates prepared using an extruder under vacuum appear very smooth, and the multiparticulates tend to be more robust than those multiparticulates prepared without vacuum. In certain formulations, the use of extrusion under vacuum provides an extruded multiparticulate product which is more pH-dependent than its counterpart formulation prepared without vacuum. Alternatively, the melt-extruded product is prepared using a Werner-Pfleiderer twin screw extruder.

In certain embodiments, a spheronizing agent is added to a granulate or multiparticulates of the present invention and then spheronized to produce controlled release spheroids. The spheroids are then optionally overcoated with a controlled release coating by methods such as those described herein.

Spheronizing agents which may be used to prepare the multiparticulate formulations of the present invention include any art-known spheronizing agent. Cellulose derivatives are preferred, and microcrystalline cellulose is especially preferred. A suitable microcrystalline cellulose is, for example, the material sold as Avicel® PH 101. The spheronizing agent is preferably included as about 1 to about 99% of the multiparticulate by weight.

In addition to the active ingredient and spheronizing agent, the spheroids may also contain a binder. Suitable binders, such as low viscosity, water soluble polymers, will be well known to those skilled in the pharmaceutical art. However, water soluble hydroxy lower alkylcellulose, such as hydroxypropylcellulose, are preferred.

In addition to the buprenorphine and spheronizing agent, the multiparticulate formulations of the present invention may include a controlled release material such as those described hereinabove. Preferred controlled-release materials for inclusion in the multiparticulate formulations include acrylic and methacrylic acid polymers or copolymers, and ethylcellulose. When present in the formulation, the controlled-release material will be included in amounts of from about 1 to about 80% of the multiparticulate, by weight. The controlled-release material is preferably included in the multiparticulate formulation in an amount effective to provide controlled release of the buprenorphine from the multiparticulate.

Pharmaceutical processing aids such as binders, diluents, and the like may be included in the multiparticulate formulations. Amounts of these agents included in the formulations will vary with the desired effect to be exhibited by the formulation.

Specific examples of pharmaceutically acceptable carriers and excipients that may be used to formulate oral dosage forms of the present invention are described in the Handbook of Pharmaceutical Excipients, APhA Publications; 5 edition (Jan. 5, 2006) incorporated by reference herein.

The multiparticulates may be overcoated with a controlled-release coating including a controlled-release material such as those described hereinabove. The controlled-release coating is applied to a weight gain of from about 5 to about 30%. The amount of the controlled-release coating to be applied will vary according to a variety of factors, e.g., the composition of the multiparticulate and the chemical and/or physical properties of the drug.

Matrix multiparticulates may also be prepared by granulating the spheronizing agent together with the buprenorphine, e.g. by wet granulation. The granulate is then spheronized to produce the matrix multiparticulates. The matrix multiparticulates are then optionally overcoated with the controlled release coating by methods such as those described hereinabove.

Another method for preparing matrix multiparticulates, for example, by (a) forming granules comprising at least one water soluble hydroxyalkyl cellulose and the buprenorphine or the buprenorphine salt; (b) mixing the hydroxyalkyl cellulose containing granules with at least one C₁₂-C₃₆ aliphatic alcohol; and (c) optionally, compressing and shaping the granules. Preferably, the granules are formed by wet granulating the hydroxyalkyl cellulose/buprenorphine with water. In a particularly preferred embodiment of this process, the amount of water added during the wet granulation step is preferably between 1.5 and 5 times, especially between 1.75 and 3.5 times, the dry weight of the buprenorphine.

In yet other alternative embodiments, a spheronizing agent, together with the active ingredient can be spheronized to form spheroids. Microcrystalline cellulose is preferred. A suitable microcrystalline cellulose is, for example, the material sold as Avicel® PH 101. In such embodiments, in addition to the active ingredient and spheronizing agent, the spheroids may also contain a binder. Suitable binders, such as low viscosity, water soluble polymers, will be well known to those skilled in the pharmaceutical art. However, water soluble hydroxy lower alkyl cellulose, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate co-polymer, or ethyl cellulose. In such embodiments, the sustained-release coating will generally include a water insoluble material such as (a) a wax, either alone or in admixture with a fatty alcohol; or (b) shellac or zein.

Spheroids of the present invention comprise a matrix formulation as described above or bead formulation as described hereinafter having a diameter of between 0.1 mm and 2.5 mm, especially between 0.5 mm and 2 mm.

The spheroids are preferably film coated with a controlled release material that permits release of the buprenorphine (or salt) at a controlled rate in an aqueous medium. The film coat is chosen so as to achieve, in combination with the other stated properties, the in-vitro release rate outlined above (e.g., at least about 12.5% released after 1 hour). The controlled-release coating formulations of the present invention preferably produce a strong, continuous film that is smooth and elegant, capable of supporting pigments and other coating additives, non-toxic, inert, and tack-free.

Preparation of Coated Bead Formulations

In certain preferred embodiments of the present invention the oral solid controlled release dosage form of the present invention comprises a plurality of coated substrates, e.g., inert pharmaceutical beads such as nu pariel 18/20 beads. An aqueous dispersion of hydrophobic material is used to coat the beads to provide for the controlled release of the buprenorphine. In certain preferred embodiments a plurality of the resultant stabilized solid controlled-release beads may be placed in a gelatin capsule in an amount sufficient to provide an effective controlled-release dose when ingested and contacted by an environmental fluid, e.g., gastric fluid or dissolution media.

The stabilized controlled-release bead formulations of the present invention slowly release the buprenorphine, e.g., when ingested and exposed to gastric fluids, and then to intestinal fluids. The controlled-release profile of the formulations of the invention can be altered, for example, by varying the amount of overcoating with the aqueous dispersion of hydrophobic controlled release material, altering the manner in which the plasticizer is added to the aqueous dispersion of hydrophobic controlled release material, by varying the amount of plasticizer relative to hydrophobic controlled release material, by the inclusion of additional ingredients or excipients, by altering the method of manufacture, etc. The dissolution profile of the ultimate product may also be modified, for example, by increasing or decreasing the thickness of the controlled release coating.

Substrates coated with a therapeutically active agent are prepared, e.g. by dissolving the therapeutically active agent in water and then spraying the solution onto a substrate, for example, nu pariel 18/20 beads, using a Wuster insert. Optionally, additional ingredients are also added prior to coating the beads in order to assist the binding of the buprenorphine to the beads, and/or to color the solution, etc. For example, a product which includes hydroxypropyl methylcellulose, etc. with or without colorant (e.g., Opadry®) may be added to the solution and the solution mixed (e.g., for about 1 hour) prior to application of the same onto the substrate. The resultant coated substrate may then be optionally overcoated with a barrier agent, to separate the therapeutically active agent from the hydrophobic controlled-release coating.

An example of a suitable barrier agent is one which comprises hydroxypropyl methylcellulose. However, any film-former known in the art may be used. It is preferred that the barrier agent does not affect the dissolution rate of the final product.

The substrates may then be overcoated with an aqueous dispersion of the hydrophobic controlled release material as described herein. The aqueous dispersion of hydrophobic controlled release material preferably further includes an effective amount of plasticizer, e.g. tri-ethyl citrate. Pre-formulated aqueous dispersions of ethylcellulose, such as Aquacoat® or Surelease®, may be used. If Surelease® is used, it is not necessary to separately add a plasticizer. Alternatively, pre-formulated aqueous dispersions of acrylic polymers such as Eudragit® can be used.

The coating solutions of the present invention preferably contain, in addition to the film-former, plasticizer, and solvent system (i.e., water), a colorant to provide elegance and product distinction. Color may be added to the solution of the therapeutically active agent instead, or in addition to the aqueous dispersion of hydrophobic material. For example, color can be added to Aquacoat® via the use of alcohol or propylene glycol based color dispersions, milled aluminum lakes and opacifiers such as titanium dioxide by adding color with shear to water soluble polymer solution and then using low shear to the plasticized Aquacoat®. Alternatively, any suitable method of providing color to dioxide and color pigments, such as iron oxide pigments. The incorporation of pigments, may, however, increase the retard effect of the coating.

The plasticized aqueous dispersion of hydrophobic controlled release material may be applied onto the substrate comprising the therapeutically active agent by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the aqueous dispersion of hydrophobic material to obtain a predetermined controlled-release of said therapeutically active agent when said coated substrate is exposed to aqueous solutions, e.g. gastric fluid, is preferably applied, taking into account the physical characteristics of the therapeutically active agent, the manner of incorporation of the plasticizer, etc. After coating with the hydrophobic controlled release material, a further overcoat of a film-former, such as Opadry®, is optionally applied to the beads. This overcoat is provided, if at all, in order to substantially reduce agglomeration of the beads.

Another method of producing controlled release bead formulations suitable for about 24-hour administration is via powder layering. The powder-layered beads are prepared by spraying an aqueous binder solution onto inert beads to provide a tacky surface, and subsequently spraying a powder that is a homogenous mixture of the buprenorphine and hydrous lactose impalpable onto the tacky beads. The beads are then dried and coated with a hydrophobic material such as those described hereinabove to obtain the desired release of drug when the final formulation is exposed to environmental fluids. An appropriate amount of the controlled release beads are then, e.g. encapsulated to provide a final dosage form which provides effective plasma concentrations for the intended duration of effect or dosing frequency.

Controlled Release Osmotic Dosage

In another embodiment, the modified release formulations of the present invention are provided as osmotic pump dosage forms. In an osmotic pump dosage form, a core containing the buprenorphine and optionally one or more osmotic excipients is typically encased by a selectively permeable membrane having at least one orifice. The selectively permeable membrane is generally permeable to water, but impermeable to the drug. When the system is exposed to GI fluids, water penetrates through the selectively permeable membrane into the core containing the drug and optional osmotic excipients. The osmotic pressure increases within the dosage form. Consequently, the drug is released through the orifice in an attempt to equalize the osmotic pressure across the selectively permeable membrane. In more complex pumps, the dosage form may contain two internal compartments in the core. The first compartment contains the drug and the second compartment may contain a polymer, which swells on contact with aqueous fluid. After ingestion, this polymer swells into the drug-containing compartment, diminishing the volume occupied by the drug, thereby delivering the drug from the device at a controlled rate over an extended period of time. Such dosage forms are often used when a zero order release profile is desired.

Osmotic pumps are well known in the art. The osmotic pumps useful in accordance with the present invention may be formed by compressing a tablet of an osmotically active drug, or an osmotically inactive drug in combination with an osmotically active agent, and then coating the tablet with a selectively permeable membrane which is permeable to an exterior aqueous-based fluid but impermeable to the drug and/or osmotic agent.

One or more delivery orifices may be drilled through the selectively permeable membrane wall. Alternatively, one or more orifices in the wall may be formed by incorporating leachable pore-forming materials in the wall. In operation, the exterior aqueous-based fluid is imbibed through the selectively permeable membrane wall and contacts the drug to form a solution or suspension of the drug. The drug solution or suspension is then pumped out through the orifice as fresh fluid is imbibed through the selectively permeable membrane.

Typical materials for the selectively permeable membrane include selectively permeable polymers known in the art to be useful in osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanlate, cellulose acetate valerate, cellulose acetate succinate, cellulose propionate succinate, methyl cellulose, cellulose acetate p-toluene sulfonate, cellulose acetate butyrate, lightly cross-linked polystyrene derivatives, cross-linked poly(sodium styrene sulfonate), poly(vinylbenzyltrimethyl ammonium chloride), cellulose acetate, cellulose diacetate, cellulose triacetate, and/or mixtures thereof.

The osmotic agents that can be used in the pump are typically soluble in the fluid that enters the device following administration, resulting in an osmotic pressure gradient across the selectively permeable wall against the exterior fluid. Suitable osmotic agents include, but are not limited to, magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, hydrophilic polymers such as cellulose polymers, and/or mixtures thereof.

The osmotic pump dosage form may contain a second compartment containing a swellable polymer. Suitable swellable polymers typically interact with water and/or aqueous biological fluids, which causes them to swell or expand to an equilibrium state. Acceptable polymers exhibit the ability to swell in water and/or aqueous biological fluids, retaining a significant portion of such imbibed fluids within their polymeric structure, so as into increase the hydrostatic pressure within the dosage form. The polymers may swell or expand to a very high degree, usually exhibiting a 2- to 40-fold volume increase. The polymers can be non-cross-linked or cross-linked. In one embodiment, the swellable polymers are hydrophilic polymers. Suitable polymers include, but are not limited to, poly(hydroxy alkyl methacrylate) having a molecular weight of from 30,000 to 5,000,000; kappa-carrageenan; polyvinylpyrrolidone having a molecular weight of from 10,000 to 360,000; anionic and cationic hydrogels; polyelectrolyte complexes; poly(vinyl alcohol) having low amounts of acetate, cross-linked with glyoxal, formaldehyde, or glutaraldehyde, and having a degree of polymerization from 200 to 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; and/or mixtures of any of the foregoing.

The term “orifice” as used herein comprises means and methods suitable for releasing the drug from the dosage form. The expression includes one or more apertures or orifices that have been bored through the selectively permeable membrane by mechanical procedures. Alternatively, an orifice may be formed by incorporating an erodible element, such as a gelatin plug, in the selectively permeable membrane. In such cases, the pores of the selectively permeable membrane form a “passageway” for the passage of the drug. Such passageway formulations are described in the art.

The osmotic pumps useful in accordance with this invention may be manufactured by techniques known in the art. For example, the drug and other ingredients may be milled together and pressed into a solid having the desired dimensions (e.g., corresponding to the first compartment). The swellable polymer is then formed, placed in contact with the drug, and both are surrounded with the selectively permeable agent. If desired, the drug component and polymer component may be pressed together before applying the selectively permeable membrane. The selectively permeable membrane may be applied by any suitable method, for example, by molding, spraying, or dipping.

Controlled release dosage forms according to the present invention may also be prepared as osmotic dosage formulations. The osmotic dosage forms preferably include a bilayer core comprising a drug layer and a delivery or push layer, wherein the bilayer core is surrounded by a semipermeable wall and optionally having at least one passageway disposed therein. In certain embodiments, the bilayer core comprises a drug layer with the buprenorphine or a salt thereof and a displacement or push layer. In certain preferred embodiments the drug layer may also comprise at least one polymer hydrogel. The polymer hydrogel may have an average molecular weight of between about 500 and about 6,000,000. Examples of polymer hydrogels include but are not limited to a maltodextrin polymer comprising the formula (C₆H₁₂O₅)ₙ. H2O, wherein n is 3 to 7,500, and the maltodextrin polymer comprises a 500 to 1,250,000 number-average molecular weight; a poly(alkylene oxide) represented by, e.g., a poly(ethylene oxide) and a poly(propylene oxide) having a 50,000 to 750,000 weight-average molecular weight, and more specifically represented by a poly(ethylene oxide) of at least one of 100,000, 200,000, 300,000 or 400,000 weight-average molecular weights; an alkali carboxyalkylcellulose, wherein the alkali is sodium or potassium, the alkyl is methyl, ethyl, propyl, or butyl of 10,000 to 175,000 weight-average molecular weight; and a copolymer of ethylene-acrylic acid, including methacrylic and ethacrylic acid of 10,000 to 500,000 number-average molecular weight.

In certain preferred embodiments of the present invention, the delivery or push layer comprises an osmopolymer. Examples of an osmopolymer include but are not limited to a member selected from the group consisting of a polyalkylene oxide and a carboxyalkylcellulose. The polyalkylene oxide possesses a 1,000,000 to 10,000,000 weight-average molecular weight. The polyalkylene oxide may be a member selected from the group consisting of polymethylene oxide, polyethylene oxide, polypropylene oxide, polyethylene oxide having a 1,000,000 average molecular weight, polyethylene oxide comprising a 5,000,000 average molecular weight, polyethylene oxide comprising a 7,000,000 average molecular weight, cross-linked polymethylene oxide possessing a 1,000,000 average molecular weight, and polypropylene oxide of 1,200,000 average molecular weight. Typical osmopolymer carboxyalkylcellulose comprises a member selected from the group consisting of alkali carboxyalkylcellulose, sodium carboxymethylcellulose, potassium carboxymethylcellulose, sodium carboxyethylcellulose, lithium carboxymethylcellulose, sodium carboxyethylcellulose, carboxyalkylhydroxyalkylcellulose, carboxymethylhydroxyethyl cellulose, carboxyethylhydroxyethylcellulose and carboxymethylhydroxypropylcellulose. The osmopolymers used for the displacement layer exhibit an osmotic pressure gradient across the semipermeable wall. The osmopolymers imbibe fluid into dosage form, thereby swelling and expanding as an osmotic hydrogel (also known as osmogel), whereby they push the buprenorphine or pharmaceutically acceptable salt thereof from the osmotic dosage form.

The push layer may also include one or more osmotically effective compounds also known as osmagents and as osmotically effective solutes. They imbibe an environmental fluid, for example, from the gastrointestinal tract, into dosage form and contribute to the delivery kinetics of the displacement layer. Examples of osmotically active compounds comprise a member selected from the group consisting of osmotic salts and osmotic carbohydrates. Examples of specific osmagents include but are not limited to sodium chloride, potassium chloride, magnesium sulfate, lithium phosphate, lithium chloride, sodium phosphate, potassium sulfate, sodium sulfate, potassium phosphate, glucose, fructose and maltose.

The push layer may optionally include a hydroxypropylalkylcellulose represented by a member selected from the group consisting of hydroxypropylmethylcellulose, hydroxypropylethylcellulose, hydroxypropyl isopropylcellulose, hydroxypropylbutylcellulose, and hydroxypropyl pentylcellulose.

The push layer optionally may comprise a nontoxic colorant or dye. Examples of colorants or dyes include but are not limited to Food and Drug Administration Colorant (FD&C), such as FD&C No. 1 blue dye, FD&C No. 4 red dye, red ferric oxide, yellow ferric oxide, titanium dioxide, carbon black, and indigo.

The push layer which is devoid of buprenorphine may also optionally comprise an antioxidant to inhibit the oxidation of the excipients of the push layer. Some examples of antioxidants include but are not limited to a member selected from the group consisting of ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, and propylgallate.

In certain alternative embodiments, the dosage form comprises an homogenous core comprising the buprenorphine or a pharmaceutically acceptable salt thereof, a pharmaceutically acceptable polymer (e.g., polyethylene oxide), optionally a disintegrant (e.g., polyvinylpyrrolidone), optionally an absorption enhancer (e.g., a fatty acid, a surfactant, a chelating agent, a bile salt, etc.). The homogenous core is surrounded by a semipermeable wall having a passageway (as defined above) for the release of the buprenorphine or pharmaceutically acceptable salt thereof.

In certain embodiments, the semipermeable wall comprises a member selected from the group consisting of a cellulose ester polymer, a cellulose ether polymer and a cellulose ester-ether polymer. Representative wall polymers comprise a member selected from the group consisting of cellulose acylate, cellulose diacylate, cellulose triacylate, cellulose acetate, cellulose diacetate, cellulose triacetate, mono-, di- and tricellulose alkenylates, and mono-, di- and tricellulose alkinylates. The poly(cellulose) used for the present invention comprises a number-average molecular weight of 20,000 to 7,500,000.

Additional semipermeable polymers for the purpose of this invention comprise acetaldehyde dimethycellulose acetate, cellulose acetate ethylcarbamate, cellulose acetate methylcarbamate, cellulose diacetate, propylcarbamate, cellulose acetate diethylaminoacetate; semipermeable polyamide; semipermeable polyurethane; semipermeable sulfonated polystyrene; semipermeable cross-linked polymer formed by the coprecipitation of a polyanion and a polycation as disclosed in U.S. Pat. Nos. 3,173,876; 3,276,586; 3,541,005; 3,541,006 and 3,546,876; semipermeable polymers as disclosed by Loeb and Sourirajan in U.S. Pat. No. 3,133,132; semipermeable crosslinked polystyrenes; semipermeable cross-linked poly(sodium styrene sulfonate); semipermeable crosslinked poly(vinylbenzyltrimethyl ammonium chloride); and semipermeable polymers possessing a fluid permeability of 2.5×10⁻⁸ to 2.5×10⁻² (cm²/hr·atm) expressed per atmosphere of hydrostatic or osmotic pressure difference across the semipermeable wall. Other polymers useful in the present invention are known in the art in U.S. Pat. Nos. 3,845,770; 3,916,899 and 4,160,020; and in Handbook of Common Polymers, Scott, J. R. and W. J. Roff, 1971, CRC Press, Cleveland, Ohio.

In certain embodiments, preferably the semipermeable wall is nontoxic, inert, and it maintains its physical and chemical integrity during the dispensing life of the drug. In certain embodiments, the dosage form comprises a binder as described above.

In certain embodiments, the dosage form comprises a lubricant, which may be used during the manufacture of the dosage form to prevent sticking to die wall or punch faces. Examples of lubricants include but are not limited to magnesium stearate, sodium stearate, stearic acid, calcium stearate, magnesium oleate, oleic acid, potassium oleate, caprylic acid, sodium stearyl fumarate, and magnesium palmitate.

Coatings

The dosage forms of the present invention may optionally be coated with one or more coatings suitable for the regulation of release or for the protection of the formulation. In one embodiment, coatings are provided to permit either pH-dependent or pH-independent release, e.g., when exposed to gastrointestinal fluid. When a pH-independent coating is desired, the coating is designed to achieve optimal release regardless of pH-changes in the environmental fluid, e.g., the GI tract. Other preferred embodiments include a pH-dependent coating that releases the buprenorphine in desired areas of the gastrointestinal (GI) tract, e.g., the stomach or small intestine, such that an absorption profile is provided which is capable of providing at least about twelve hour and preferably up to twenty-four hour analgesia to a patient. It is also possible to formulate compositions which release a portion of the dose in one desired area of the GI tract, e.g., the stomach, and release the remainder of the dose in another area of the GI tract, e.g., the small intestine.

Formulations according to the invention that utilize pH-dependent coatings may also impart a repeat-action effect whereby unprotected drug is coated over an enteric coat and is released in the stomach, while the remainder, being protected by the enteric coating, is released further down the gastrointestinal tract. Coatings which are pH-dependent may be used in accordance with the present invention include a controlled release material such as, e.g., shellac, cellulose acetate phthalate (CAP), polyvinyl acetate phthalate (PVAP), hydroxypropyl methylcellulose phthalate, and methacrylic acid ester copolymers, zein, and the like.

In another preferred embodiment, the present invention is related to a stabilized solid controlled dosage form comprising the buprenorphine coated with a hydrophobic controlled release material selected from (i) an alkylcellulose; (ii) an acrylic polymer; or (iii) mixtures thereof. The coating may be applied in the form of an organic or aqueous solution or dispersion.

In certain preferred embodiments, the controlled release coating is derived from an aqueous dispersion of the hydrophobic controlled release material. The coated substrate containing the buprenorphine (e.g., a tablet core or inert pharmaceutical beads or spheroids) is then cured until an endpoint is reached at which the substrate provides a stable dissolution. The curing endpoint may be determined by comparing the dissolution profile (curve) of the dosage form immediately after curing to the dissolution profile (curve) of the dosage form after exposure to accelerated storage conditions of, e.g., at least one month at a temperature of 40° C. and a relative humidity of 75%.

In preferred embodiments, the controlled release coatings include a plasticizer such as those described herein.

In certain embodiments, it is necessary to overcoat the substrate comprising the buprenorphine with a sufficient amount of the aqueous dispersion of e.g., alkylcellulose or acrylic polymer, to obtain a weight gain level from about 2 to about 50%, e.g., about 2 to about 25% in order to obtain a controlled-release formulation. The overcoat may be lesser or greater depending upon the physical properties of the therapeutically active agent and the desired release rate, the inclusion of plasticizer in the aqueous dispersion and the manner of incorporation of the same, for example.

Alkylcellulose Polymers

Cellulosic materials and polymers, including alkylcelluloses are controlled release materials well suited for coating the substrates, e.g., beads, tablets, etc. according to the invention. Simply by way of example, one preferred alkylcellulosic polymer is ethylcellulose, although the artisan will appreciate that other cellulose and/or alkylcellulose polymers may be readily employed, singly or on any combination, as all or part of a hydrophobic coating according to the invention.

One commercially-available aqueous dispersion of ethylcellulose is Aquacoat®. Aquacoat® is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the Aquacoat® with a suitable plasticizer prior to use.

Another aqueous dispersion of ethylcellulose is commercially available as Surelease®. This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly onto substrates.

Acrylic Polymers

In other preferred embodiments of the present invention, the controlled release material comprising the controlled-release coating is a pharmaceutically acceptable acrylic polymer, including but not limited to acrylic acid and methacrylic acid copolymers, methyl methacrylate copolymers, ethoxyethyl methacrylates, cynaoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In certain preferred embodiments, the acrylic polymer is comprised of one or more ammonio methacrylate copolymers Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit®. There are several different types of Eudragit®. For example, Eudragit® E is an example of a methacrylic acid copolymer which swells and dissolves in acidic media. Eudragit® L is a methacrylic acid copolymer which does not swell at about pH <5.7 and is soluble at about pH>6. Eudragit® S does not swell at about pH <6.5 and is soluble at about pH>7. Eudragit® RL and Eudragit® RS are water swellable, and the amount of water absorbed by these polymers is pH-dependent, however, dosage forms coated with Eudragit® RL and RS are pH-independent.

In certain preferred embodiments, the acrylic coating comprises a mixture of two acrylic resin lacquers commercially available as Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids.

The Eudragit® RL/RS dispersions of the present invention may be mixed together in any desired ratio in order to ultimately obtain a controlled-release formulation having a desirable dissolution profile. Desirable controlled-release formulations may be obtained, for instance, from a retardant coating derived from 100% Eudragit® RL, 50% Eudragit® RL and 50% Eudragit® RS, and 10% Eudragit® RL:Eudragit® 90% RS. Of course, one skilled in the art will recognize that other acrylic polymers may also be used, such as, for example, Eudragit® L.

Plasticizers

In embodiments of the present invention where the coating comprises an aqueous dispersion of a hydrophobic controlled release material, the inclusion of an effective amount of a plasticizer in the aqueous dispersion of hydrophobic material will further improve the physical properties of the controlled-release coating. For example, because ethylcellulose has a relatively high glass transition temperature and does not form flexible films under normal coating conditions, it is preferable to incorporate a plasticizer into an ethylcellulose coating containing controlled-release coating before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tibutyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

In certain embodiments, the addition of a small amount of talc to the controlled release coating reduces the tendency of the aqueous dispersion to stick during processing, and acts as a polishing agent.

The release of the therapeutically active agent from the controlled-release formulation of the present invention can be further influenced, i.e., adjusted to a desired rate, by the addition of one or more release-modifying agents, or by providing one or more passageways through the coating. The ratio of hydrophobic controlled release material to water soluble material is determined by, among other factors, the release rate required and the solubility characteristics of the materials selected.

The release-modifying agents which function as pore-formers may be organic or inorganic, and include materials that can be dissolved, extracted or leached from the coating in the environment of use. The pore-formers may comprise one or more hydrophilic materials such as hydroxypropylmethylcellulose.

The controlled-release coatings of the present invention can also include erosion-promoting agents such as starch and gums.

The controlled-release coatings of the present invention can also include materials useful for making microporous lamina in the environment of use, such as polycarbonates comprised of linear polyesters of carbonic acid in which carbonate groups reoccur in the polymer chain.

The release-modifying agent may also comprise a semi-permeable polymer. In certain preferred embodiments, the release-modifying agent is selected from hydroxypropylmethylcellulose, lactose, metal stearates, and mixtures of any of the foregoing.

The controlled-release coatings of the present invention may also include an exit means comprising at least one passageway, orifice, or the like. The passageway may be formed by such methods as those disclosed in U.S. Pat. Nos. 3,845,770; 3,916,889; 4,063,064; and 4,088,864, all of which are hereby incorporated by reference. The passageway can have any shape such as round, triangular, square, elliptical, irregular, etc.

These and other embodiments of the present invention will readily occur to those of ordinary skill in the art in view of the disclosure herein.

A wide variety of materials can be used for preparing the dosage form according to this invention. This invention therefore contemplates the use of materials other than those specifically disclosed herein, including those which may hereafter become known to the art to be capable of performing the necessary functions.

### DETAILED DESCRIPTION OF THE PREFERRED EMBODIMENTS

The following examples illustrate various aspects of the present invention. They are not to be construed to limit the claims in any manner whatsoever. A wide variety of methods known in the art for the preparation of oral immediate release and oral controlled release dosage forms may be incorporated into the invention. Other suitable dosage forms may also be prepared by modification of the examples herein and by use of material other than those specifically disclosed herein, including those which may hereafter become known to the art to be capable of performing the necessary functions. Other suitable modifications and adaptations of the variety of conditions and parameters normally encountered and obvious to those skilled in the art are within the spirit and scope of the invention.

The percent loading of the buprenorphine onto the dosage form may be varied depending on the physiochemical and pharmaceutical properties of immediate release and controlled release material, excipients, the selected salt of buprenorphine and the desired release profile and duration of actions.

The ingredients used for the preparation of the buprenorphine dosage form agent may be modified depending on the selection, dose and desired duration of effect. In some embodiments, a change in the dose or amount buprenorphine will not require a significant change in amount of other ingredients. In other embodiments, a proportional change in the amount of other ingredients is required to maintain the desired properties. In yet other embodiments, a change in the dose or amount buprenorphine necessitates a change in the nature and/or amount of ingredients to provide the required characteristics of the buprenorphine (e.g., duration of effect, rate and extent of absorption, therapeutic concentrations and effect, etc.).

### EXAMPLES

### Example 1

Tablet Composition of Extended Release Buprenorphine HCl

| Ingredients | Qty./Unit |
|---|---|
| 1. Buprenorphine HCl | 20 mg |
| 2. HPMC 2208, USP | 150 mg |
| 3. Carnauba wax | 30 mg |
| 4. HPMC 2910, USP | 15 mg |
| 5. Magnesium Stearate | 2 mg |
| 6. Stearic acid | 8 mg |
| 7. Talc | 3 mg |

Place the ingredients 1, 2 and 3 in the granulator and mix for 15 minutes. Dissolve ingredient 4 in water (mix in hot water, then cool down) and spray into the fluidized mixture. Dry to approximately 5% moisture. Sequentially add ingredient 5, 6 and 7, with mixing steps between each addition. Compress using capsule shaped tooling.

### Example 2

Tablet Composition of Extended Release Buprenorphine HCl

| Ingredients | Qty./Unit |
|---|---|
| 1. Buprenorphine HCl | 100 mg |
| 2. HPMC 2208, USP | 250 mg |
| 3. Carnauba wax | 50 mg |
| 4. HPMC 2910, USP | 25 mg |
| 5. Magnesium Stearate | 4 mg |
| 6. Stearic acid | 14 mg |
| 7. Talc | 5 mg |

Place the ingredients 1, 2 and 3 in the granulator and mix for 15 minutes. Dissolve ingredient 4 in water (mix in hot water, then cool down) and spray into the fluidized mixture. Dry to approximately 5% moisture. Sequentially add ingredient 5, 6 and 7, with mixing steps between each addition. Compress using capsule shaped tooling.

### Example 3

Tablet Composition of Extended Release Buprenorphine HCl

| Ingredients | Qty./Unit |
|---|---|
| 1. Buprenorphine HCl | 500 mg |
| 2. HPMC 2208, USP | 250 mg |
| 3. Carnauba wax | 50 mg |
| 4. HPMC 2910, USP | 25 mg |
| 5. Magnesium Stearate | 4 mg |
| 6. Stearic acid | 14 mg |
| 7. Talc | 5 mg |

Place the ingredients 1, 2 and 3 in the granulator and mix for 15 minutes. Dissolve ingredient 4 in water (mix in hot water, then cool down) and spray into the fluidized mixture. Dry to approximately 5% moisture. Sequentially add ingredient 5, 6 and 7, with mixing steps between each addition. Compress using capsule shaped tooling.

### Example 4

Tablet Composition of Extended Release
Buprenorphine Base

| Ingredients | Amt/Unit (mg) |
|---|---|
| Buprenorphine Base | 20 |
| Spray Dried Lactose | 60 |
| Povidone | 5 |
| Eudragit RS30D (solids) | 10 |
| Triacetin | 2 |
| Stearyl Alcohol | 25 |
| Talc | 2.5 |
| Magnesium Stearate | 1.25 |
| Opadry Pink Y-S-14518A | 4.0 |

1. Granulation: Spray the Eudragit/Triacetin dispersion onto the Buprenorphine, Spray Dried Lactose and Povidone using a fluid bed granulator. 2. Milling: Discharge the granulation and pass through a mill. 3. Waxing: Melt the stearyl alcohol and add to the milled granulation using a mixer. Allow to cool. 4. Milling: Pass the cooled granulation through a mill. 5. Lubrication: Lubricate the granulation with talc and magnesium stearate using a mixer. 6. Compression: Compress the granulation into tablets using a tablet press. 7. Film coating: Apply an aqueous film coat to the tablets.

### Example 5

Tablet Composition of Extended Release
Buprenorphine HCl

| Ingredients | Amt/Unit (mg) |
|---|---|
| Buprenorphine HCl | 30 |
| Spray Dried Lactose | 60 |
| Povidone | 5 |
| Eudragit RS30D (solids) | 10 |
| Triacetin | 2 |
| Stearyl Alcohol | 25 |
| Talc | 2.5 |
| Magnesium Stearate | 1.25 |
| Opadry Pink Y-S-14518A | 4.0 |

1. Granulation: Spray the Eudragit/Triacetin dispersion onto the Buprenorphine, Spray Dried Lactose and Povidone using a fluid bed granulator. 2. Milling: Discharge the granulation and pass through a mill. 3. Waxing: Melt the stearyl alcohol and add to the milled granulation using a mixer. Allow to cool. 4. Milling: Pass the cooled granulation through a mill. 5. Lubrication: Lubricate the granulation with talc and magnesium stearate using a mixer. 6. Compression: Compress the granulation into tablets using a tablet press. 7. Film coating: Apply an aqueous film coat to the tablets.

### Example 6

Capsule Composition of Extended Release
Buprenorphine Base

| Ingredients | Amt/Unit (mg) |
|---|---|
| Buprenorphine Base | 20 |
| Eudragit RSPO | 76 |
| Eudragit RLPO | 4 |
| Stearyl Alcohol | 25 |

1. Blend milled Stearyl Alcohol, Eudragit RLPO, Buprenorphine, and Eudragit RSPO using a Hobart Mixer. 2. Extrude the granulation using a Powder Feeder, Melt Extruder (equipped with the 6×1 mm die head), Conveyor, Lasermike, and Pelletizer. Powder feed rate-40 g/min; vacuum—about 980 mBar; Conveyor, such that diameter of extrudate is 1 mm, Pelletizer, such that pellets are cut to 1 mm in length. Screen pellets using #16 mesh and #20 mesh screens. Collect material that passes through the #16 mesh screen and is retained on the #20 mesh screen. 4. Fill capsules with the pellets.

### Example 7

Capsule Composition of Extended Release
Buprenorphine Base

| Ingredients | Amt/Unit (mg) |
|---|---|
| Buprenorphine Base | 200 |
| Eudragit RSPO | 150 |
| Eudragit RLPO | 10 |
| Stearyl Alcohol | 40 |

1. Blend milled Stearyl Alcohol, Eudragit RLPO, Buprenorphine, and Eudragit RSPO using a Hobart Mixer. 2. Extrude the granulation using a Powder Feeder, Melt Extruder (equipped with the 6×1 mm die head), Conveyor, Lasermike, and Pelletizer. Powder feed rate-40 g/min; vacuum—about 980 mBar; Conveyor, such that diameter of extrudate is 1 mm, Pelletizer, such that pellets are cut to 1 mm in length. Screen pellets using #16 mesh and #20 mesh screens. Collect material that passes through the #16 mesh screen and is retained on the #20 mesh screen. 4. Fill capsules with the pellets.

### Example 8

Capsule Composition of Extended Release
Buprenorphine Base

| Ingredients | Amt/Unit (mg) |
|---|---|
| Buprenorphine Base | 15 |
| Eudragit RSPO | 77 |
| Ethocel | 4.5 |
| Stearic acid | 27 |

Blend milled Stearic acid, Ethocel, Buprenorphine Base, and Eudragit RSPO using a V-blender. 2. Extrude the mixture using a Powder Feeder, Melt Extruder (equipped with the 6×1 mm die head), Conveyor, Lasermike, and Pelletizer. Powder feed rate, 1.2 kg/hr; vacuum, about 980 mBar; Conveyor, such that diameter of extrudate is 1 mm; Pelletizer, such that pellets are cut to 1 mm in length. 3. Screen pellets using #16 mesh and #20 mesh screens. Collect material that passes through the #16 mesh screen and is retained on the #20 mesh screen. Fill pellets in capsules.

### Example 9

Capsule Composition of Extended Release Buprenorphine HCl

| Steps | Ingredients | Amt/unit (mg) |
|---|---|---|
| 1 | Buprenorphine HCl | 12 |
| | Non-pareil beads (30/35 mesh) | 45 |
| | Opadry Clear | 2.5 |
| 2 | Eudragit RS3-D (dry) | 7.2 |
| | Eudragit RL30D (dry) | 0.4 |
| | Triethyl citrate | 1.5 |
| | Cabosil | 0.4 |
| 3 | Opadry Clear (HPMC) | 1.9 |
| | Cabosil | 0.28 |

1. Dissolve Buprenorphine HCl and Opadry (HPMC) in water. Spray the drug solution onto nonpareil beads in a fluid bed coater with Wurster insert. 2. Disperse Eudragit RS, Eudragit RL, triethyl citrate, and Cabosil in water. Spray the dispersion onto the beads in the fluid bed coater. 3. Dissolve Opadry in water. Spray the solution onto the beads in the fluid bed coater. 4. Cure the beads at 60° C. for 24 hours.

### Example 10

Tablet Composition of Extended Release
Buprenorphine HCl

| Ingredient | Amt/unit (mg) |
|---|---|
| Buprenorphine HCl | 75 |
| Anhydrous Dicalcium | 60 |
| Phosphate (Powdered) | 80 |
| Microcrystalline Cellulose | 80 |
| Glyceryl Behenate | 40 |
| Magnesium Stearate | 4 |
| Opadry Red | 17 |
| Purified Water | 900* |

*Remains in product as residual moisture only.

1. Pass the Stearyl Alcohol flakes through an oscillating mill. 2. Mix the Buprenorphine HCl, milled Stearyl Alcohol, Anhydrous Dicalcium Phosphate, Microcrystalline Cellulose, and Glyceryl Behenate in a twin shell blender. 3. Continuously feed the blended material into a twin screw extruder and collect the resultant heated material on a conveyor. 4. Allow the extrudate to cool on the conveyor. 5. Mill the cooled extrudate using an oscillating mill. 6. Blend the milled extrudate and Magnesium Stearate. 7. Compress the resultant granulation using a tablet press, preferably into a caplet. 8. Prepare a film coating solution by dispersing the Opadry in Purified Water and applying it to the tablet.

### Example 11

Capsule Composition of Extended Release
Buprenorphine HCl

| Ingredient | Amt/unit (mg) |
|---|---|
| Buprenorphine HCl | 20 |
| Eudragit RSPO | 76.5 |
| Ethylcellulose | 4.5 |
| Stearyl Alcohol | 27 |

1. Pass Stearyl Alcohol flakes through an impact mill. 2. Mix the Buprenorphine HCl, Eudragit, Ethylcellulose and milled Stearyl Alcohol in a twin shell blender. 3. Continuously feed the blended material into a twin screw extruder and collect the resultant strands on a conveyor. 4. Allow the strands to cool on the conveyor. 5. Cut the cooled strands into pellets using a Pelletizer. 6. Screen the pellets and collect desired sieve portion. 7. Fill the extruded pellets into capsules.

Example 12 to 23 may be prepared as follows: (i) Dispense the specified hydrophobic controlled release material (e.g., hydrogenated Type I vegetable oil, hydrogenated Type II vegetable oil, polyoxyethylene stearates, polyoxyethylene distearates, glycerol monostearate, poorly water soluble, or high melting point waxes) into a mixer; (ii) Heat until fully melted; (iii) dispense the hydroxypropyl methyl cellulose (HPMC) into the mixer; (iv) Mix until dispersed; (v) Dispense the Aerosil into the same vessel; (vi) Mix until dispersed; (vii) Dispense the buprenorphine into the same vessel; (viii) Stir thoroughly with a high shear mixer; (ix) Transfer the mix into a liquid filling machine; (x) Fill into hard gelatin (or HPMC) capsule; (xi) Optionally, transfer the capsules to a banding machine and band the capsules.

### Example 12

Capsule Composition of Extended Release Buprenorphine HCl

| Ingredients | Quantity (mg)/Dose |
|---|---|
| Sterotex ® NF | 200 |
| Fractionated coconut oil | 70 |
| Methocel ® K 15M | 81 |
| Aerosil ® COK 84 | 4 |
| Buprenorphine HCl | 50 |

### Example 13

Capsule Composition of Extended Release Buprenorphine HCl

| Ingredients | Quantity (mg)/Dose |
|---|---|
| Beeswax | 200 |
| HPMC, K15M | 80 |
| Aerosil COK 84 | 8 |
| Buprenorphine HCl | 40 |

### Example 14

Capsule Composition of Extended Release Buprenorphine Base

| Ingredients | Quantity (mg)/Dose |
|---|---|
| Sterotex NF | 150 |
| HPMC, K15M | 75 |
| Coconut oil | 75 |
| Aerosil COK 84 | 5 |
| Buprenorphine | 100 |

### Example 15

Capsule Composition of Extended Release Buprenorphine Base

| Ingredients | Quantity (mg)/Dose |
|---|---|
| Cithrol GMS | 275 |
| HPMC, K100M | 40 |
| Aerosil COK 84 | 10 |
| Buprenorphine | 75 |

### Example 16

Capsule Composition of Extended Release Buprenorphine HCl

| Ingredients | Quantity (mg)/Dose |
|---|---|
| Hydrokote 112 | 250 |
| HPMC, K15M | 60 |
| Aerosil COK 84 | 10 |
| Buprenorphine HCl | 150 |

### Example 17

Capsule Composition of Extended Release Buprenorphine Base

| Ingredients | Quantity (mg)/Dose |
|---|---|
| Beeswax | 200 |
| HPMC, Pharmacoat 606 | 62.5 |
| Aerosil COK 84 | 7.5 |
| Buprenorphine Base | 200 |

### Example 18

Capsule Composition of Extended Release Buprenorphine Base

| Ingredients | Quantity (mg)/Dose |
|---|---|
| Gelucire 50/02 | 190 |
| Methocel K 100M | 35 |
| Aerosil COK 84 | 10 |
| Buprenorphine Base | 250 |

### Example 19

Capsule Composition of Extended Release Buprenorphine Base

| Ingredients | Quantity (mg)/Dose |
|---|---|
| Cetyl alcohol | 280 |
| Methocel K 100M | 50 |
| Aerosil COK 84 | 10 |
| Buprenorphine Base | 100 |

### Example 20

Capsule Composition of Extended Release Buprenorphine

| Ingredients | Quantity (mg)/Dose |
|---|---|
| Sterotex NF | 320 |
| Methocel K 15M | 60 |
| Aerosil COK 84 | 10 |
| Buprenorphine HCl | 300 |

### Example 21

Capsule Composition of Extended Release Buprenorphine HCl

| Ingredients | Quantity (mg)/Dose |
|---|---|
| Cithrol GMS | 320 |
| Methocel K 100M | 55 |
| Aerosil COK 84 | 15 |
| Buprenorphine HCl | 150 |

### Example 22

Capsule Composition of Extended Release Buprenorphine HCl

| Ingredients | Quantity (mg)/Dose |
|---|---|
| Sterotex ® NF | 100 |
| Fractionated coconut oil | 70 |
| Beeswax | 100 |
| Methocel ® K 15M | 81 |
| Aerosil ® COK 84 | 4 |
| Buprenorphine HCl | 200 |

### Example 23

Capsule Composition of Extended Release Buprenorphine HCl

| Ingredients | Quantity (mg)/Dose |
|---|---|
| Glyceryl behenate | 135 |
| Fractionated coconut oil | 50 |
| Methocel ® K 15M | 60 |
| Aerosil ® COK 84 | 3 |
| Buprenorphine HCl | 50 |

### Example 24

Tablet Composition of Extended Release Buprenorphine HCl

| Ingredients | Quantity (mg)/Dose |
|---|---|
| Lactose (spray dried) | 230 |
| Eudragit ® RS PM | 60 |
| Purified water | q.s.* |
| Stearyl Alcohol | 90 |
| Talc | 8 |
| Magnesium Stearate | 4 |
| Buprenorphine HCl | 300 |

*Remains in product as residual moisture only.

In Example 24, the required quantities of buprenorphine HCl, spray-dried lactose, and Eudragit® RS PM are transferred into an appropriate-size mixer, and mixed for approximately 5 minutes. While the powders are mixing, the mixture is granulated with enough water to produce a moist granular mass. The granules are then dried in a fluid bed dryer at 60° C., and then passed through an 8-mesh screen. Thereafter, the granules are re-dried and pushed through a 12-mesh screen. The required quantity of stearyl alcohol is melted at approximately 60 to 70° C., and while the granules are mixing, the melted stearyl alcohol is added. The warm granules are returned to the mixer. The coated granules are removed from the mixer and allowed to cool. The granules are then passed through a 12-mesh screen. The granulate is then lubricated by mixing the required quantity of talc and magnesium stearate in a suitable blender. Tablets are compressed on a suitable tableting machine.

In some embodiments, oral immediate release compressed tablets of buprenorphine can be formulated using conventional wet granulation procedures and equipment.

### Example 25

Immediate Release Tablet Composition of Buprenorphine HCl

| Ingredients | Qty./Unit |
|---|---|
| 1. Buprenorphine HCl | 100 mg |
| 2. Polyvinylpyrrolidine | 7.5 mg |
| 3. Lactose | 30 mg |
| 4. Alcohol SD3A-2 proof | 3 mL mg |
| 5. Stearic acid | 5 mg |
| 6. Talc | 7.5 mg |
| 7. Cornstarch | 20 mg |

### Example 26

Immediate Release Tablet Composition of Buprenorphine HCl

| Ingredients | Qty./Unit |
|---|---|
| 1. Buprenorphine HCl | 50 mg |
| 2. Polyvinylpyrrolidine | 7.5 mg |
| 3. Lactose | 30 mg |
| 4. Alcohol SD3A-2 proof | 3 mL mg |
| 5. Stearic acid | 5 mg |
| 6. Talc | 7.5 mg |
| 7. Cornstarch | 20 mg |

### Example 27

Immediate Release Tablet Composition
of Buprenorphine HCl

| Ingredients | Qty./Unit | |
|---|---|---|
| 1. Buprenorphine HCl | 75 | mg |
| 2. Polyvinylpyrrolidine | 7.5 | mg |
| 3. Lactose | 30 | mg |
| 4. Alcohol SD3A-2 proof | 3 | mL mg |
| 5. Stearic acid | 5 | mg |
| 6. Talc | 7.5 | mg |
| 7. Cornstarch | 20 | mg |

In Example 25 to 27, blend 1, 2 and 3 together; pass through a 40-mesh screen. Add 4 slowly and knead well. Screen wet mass through a 4-mesh screen. Dry the granulation at 50° C. overnight. Screen the dried granulation through a 20-mesh screen. Bolt 5, 6 and 7 through a 60-mesh screen prior to mixing by tumbling with granulation. Compress using a concave punch.

In some embodiments, oral immediate release compressed tablets of buprenorphine can be formulated using conventional dry granulation procedures and equipment.

### Example 28

Tablet Composition of Buprenorphine HCl

| Ingredients | Qty./Unit | |
|---|---|---|
| 1 Buprenorphine HCl | 20 | mg |
| 2 Lactose (granular, 12-mesh) | 25 | mg |
| 3 Starch | 20 | mg |
| 4 Talc | 20 | mg |
| 5 Magnesium Stearate | 0.3 | mg |

In Example 28, mix ingredients 1 to 5 thoroughly. Compress into slugs. Grind and screen to 14- to 16-mesh granules. Recompress into tablets using a concave punch.

In some embodiments, oral sustained release compressed tablets of buprenorphine can be formulated using conventional fluid-bed granulation procedures and equipment.

### Example 29

Tablet Composition of Buprenorphine HCl

| Ingredients | Qty./Unit |
|---|---|
| 1. Buprenorphine HCl | 50 mg |
| 2. HPMC 2208, USP | 150 mg |
| 3. Carnauba wax | 30 mg |
| 4. HPMC 2910, USP | 15 mg |
| 5. Magnesium Stearate | 2 mg |
| 6. Stearic acid | 8 mg |
| 7. Talc | 3 mg |

In Example 29, place the ingredients 1, 2 and 3 in the granulator and mix for 15 minutes. Dissolve ingredient 4 in water (mix in how water, then cool down) and spay into the fluidized mixture. Dry to approximately 5% moisture. Sequentially add ingredient 5, 6 and 7, with mixing steps between each addition. Compress using capsule shaped tooling.

In some embodiments, oral buprenorphine for duodenal delivery, jejunal delivery, ileal delivery, ileo-colonic delivery or colonic delivery formulated as modified release dosage forms that provide: (i) rapid release of buprenorphine from the dosage form in the appropriate GI environment; or (ii) extended or sustained release of buprenorphine from the dosage form in the appropriate gastrointestinal environment. In some embodiments, the appropriate environment is defined by the anatomic location within the GI tract, pH at the point of release, osmotic pressure in the dosage form, osmotic pressure in the GI lumen at the point of release, level of hydration and/or the time after ingestion.

In some embodiments, an (otherwise) immediate release or a controlled release tablet dosage form may be overcoated with one or more polymers to provide buprenorphine release in the appropriate gastrointestinal environment.

In some embodiments, oral modified release capsules of buprenorphine for ileal delivery, ileal release, ileo-colonic delivery, ileo-colonic release and/or colonic delivery and colonic release for rapid release in the appropriate GI environment can be formulated using conventional wet granulation technique.

Examples 30 to 31 are capsule formulations of oral modified release capsules of buprenorphine for rapid release in the ileum or ileo-colonic region. They are formulated using conventional wet granulation procedures and equipment

### Example 30

Rapid Release Buprenorphine HCl Capsules
for Ileal or Ileo-colonic Delivery

| Ingredients | Qty./Unit | |
|---|---|---|
| 8. Buprenorphine HCl | 4 | mg |
| 9. Polyvinylpyrrolidine | 7.5 | mg |
| 10. Lactose | 30 | mg |
| 11. Alcohol SD3A-2 proof | 3 | mL mg |
| 12. Stearic acid | 5 | mg |
| 13. Talc | 7.5 | mg |
| 14. Cornstarch | 20 | mg |

### Example 31

Rapid Release Buprenorphine HCl Capsules
for Ileal or Ileo-colonic Delivery

| Ingredients | Qty./Unit | |
|---|---|---|
| 8. Buprenorphine HCl | 10 | mg |
| 9. Polyvinylpyrrolidine | 7.5 | mg |
| 10. Lactose | 30 | mg |
| 11. Alcohol SD3A-2 proof | 3 | mL mg |
| 12. Stearic acid | 5 | mg |
| 13. Talc | 7.5 | mg |
| 14. Cornstarch | 20 | mg |

### Example 32

Rapid Release Buprenorphine Base Capsules
for Ileal or Ileo-colonic Delivery

| Ingredients | Qty./Unit | |
|---|---|---|
| 8. Buprenorphine Base | 5 | mg |
| 9. Polyvinylpyrrolidine | 7.5 | mg |
| 10. Lactose | 35 | mg |
| 11. Alcohol SD3A-2 proof | 3 | mL mg |
| 12. Stearic acid | 5 | mg |
| 13. Talc | 7.5 | mg |
| 14. Cornstarch | 20 | g |

In Example 30 to 32, blend 1, 2 and 3 together; pass through a 40-mesh screen. Add 4 slowly and knead well. Screen wet mass through a 4-mesh screen. Dry the granulation at 50° C. overnight. Screen the dried granulation through a 20-mesh screen. Bolt 5, 6 and 7 through a 60-mesh screen prior to mixing by tumbling with granulation. Encapsulate in an HPMC capsule.

Prepare a composition of aqueous Eudragit L30D-55 dispersion to coat 1.3 kg HPMC capsules: Eudragit L30D-55 1509 g (solids, 453 g), Triethyl citrate 91 g (solids 91 g), Tween 80 (33%) 20 g (solids 7 g) and Distilled Water 1130 g. Spray the dispersion onto the HPMC capsules using an Accela-Cota 10. The temperature of the capsule bed during the coating process is maintained between 26 and 32° C. The mean amounts of polymer applied ranges from 5 mg/cm² to 20 mg/cm², preferably 5 mg/cm² to 10 mg/cm².

The in vitro performance of the capsules can be tested using methods know in the art and described herein. When the capsules are tested using the USP Paddle Method in 0.1N HCl (pH 1.2) for two hours, the capsules will remain intact and substantially non-releasing. Following a switch to phosphate buffer pH 6.8, the capsule dissolution is rapid and complete.

### Examples 33 to 35 are capsule formulations of oral modified release capsules of buprenorphine for rapid release in the ileo-colonic or colonic region. They are formulated using conventional wet granulation procedures and equipment

### Example 33

Rapid Release Buprenorphine HCl Capsules
for Ileo-colonic or Colonic Delivery

| Ingredients | Qty./Unit | |
|---|---|---|
| 1. Buprenorphine HCl | 2 | mg |
| 2. Polyvinylpyrrolidine | 7.5 | mg |
| 3. Lactose | 30 | mg |
| 4. Alcohol SD3A-2 proof | 3 | mL mg |
| 5. Stearic acid | 5 | mg |
| 6. Talc | 7.5 | mg |
| 7. Cornstarch | 20 | mg |

### Example 34

Rapid Release Buprenorphine HCl Capsules for Ileo-colonic or Colonic Delivery

| Ingredients | Qty./Unit | |
|---|---|---|
| 1. Buprenorphine HCl | 10 | mg |
| 2. Polyvinylpyrrolidine | 7.5 | mg |
| 3. Lactose | 30 | mg |
| 4. Alcohol SD3A-2 proof | 3 | mL mg |
| 5. Stearic acid | 5 | mg |
| 6. Talc | 7.5 | mg |
| 7. Cornstarch | 20 | mg |

### Example 35

Rapid Release Buprenorphine HCl Capsules
for Ileo-colonic or Colonic Delivery

| Ingredients | Qty./Unit | |
|---|---|---|
| 1. Buprenorphine HCl | 6 | mg |
| 2. Polyvinylpyrrolidine | 7.5 | mg |
| 3. Lactose | 30 | mg |
| 4. Alcohol SD3A-2 proof | 3 | mL mg |
| 5. Stearic acid | 5 | mg |
| 6. Talc | 7.5 | mg |
| 7. Cornstarch | 20 | g |

In Example 33 to 35, blend 1, 2 and 3 together; pass through a 40-mesh screen. Add 4 slowly and knead well. Screen wet mass through a 4-mesh screen. Dry the granulation at 50° C. overnight. Screen the dried granulation through a 20-mesh screen. Bolt 5, 6 and 7 through a 60-mesh screen prior to mixing by tumbling with granulation. Encapsulate in an HPMC capsule.

Prepare a composition of aqueous Eudragit® FS30D dispersion to coat 1.3 kg HPMC capsules: Eudragit FS30D 1207 g (solids, 362 g), Triethyl citrate 18 g (solids 18 g), Glyceryl monostearate 11 g (solids 11 g), Tween 80 (33%) 13 g (solids 4 g) and Distilled Water 728 g. Spray the dispersion onto the HPMC capsules using an Accela-Cota 10. The temperature of the capsule bed during the coating process is maintained between 26 and 32° C. The mean amounts of polymer applied ranges from 5 mg/cm² to 20 mg/cm², preferably 6 mg/cm² to 10 mg/cm².

The in vitro performance of the capsules can be tested using methods know in the art and described herein. When the capsules are tested using the USP Paddle Method in 0.1N HCl (pH 1.2) for two hours, followed by a switch to phosphate buffer pH 6.8 for one to two hours and then again, a switch to phosphate buffer pH 7.4, the capsules will remain intact and substantially non-releasing at pH 1.2 and pH 6.8 and will provide rapid and complete dissolution upon switch to pH 7.4.

In some embodiments, oral modified release capsules of buprenorphine for ileal release, ileo-colonic release and/or colonic release can be formulated using conventional dry granulation procedures and equipment.

Example 36 is a capsule formulation of oral modified release buprenorphine for rapid release in the ileo-colonic or colonic region. It is formulated using conventional dry granulation procedures and equipment.

### Example 36

Rapid Release Buprenorphine HCl Capsules
for Ileo-colonic or Colonic Delivery

| Ingredients | Qty./Unit | |
|---|---|---|
| 6 Buprenorphine HCl | 5 | mg |
| 7 Lactose (granular, 12-mesh) | 25 | mg |
| 8 Starch | 20 | mg |
| 9 Talc | 20 | mg |
| 10 Magnesium Stearate | 0.3 | mg |

In Example 36, mix ingredients 1 to 5 thoroughly. (Optionally, compress into slugs and then grind and screen to 14- to 16-mesh granules). Fill into HPMC capsules to provide the desired dose.

Prepare a composition of aqueous Eudragit® FS30D dispersion to coat 1.3 kg HPMC capsules: Eudragit FS30D 1207 g (solids, 362 g), Triethyl citrate 18 g (solids 18 g), Glyceryl monostearate 11 g (solids 11 g), Tween 80 (33%) 13 g (solids 4 g) and Distilled Water 728 g. Spray the dispersion onto the HPMC capsules using an Accela-Cota 10. The temperature of the capsule bed during the coating process is maintained between 26 and 32° C. The mean amounts of polymer applied ranges from 5 mg/cm² to 20 mg/cm², preferably 6 mg/cm² to 10 mg/cm².

The in vitro performance of the capsules can be tested using methods know in the art and described herein. When the capsules are tested using the USP Paddle Method in 0.1N HCl (pH 1.2) for two hours, followed by a switch to phosphate buffer pH 6.8 for one to two hours and then again, a switch to phosphate buffer pH 7.4, the capsules will remain intact and substantially non-releasing at pH 1.2 and pH 6.8 and provide rapid and complete dissolution at pH 7.4.

In some embodiments, oral modified release tablets or capsule of buprenorphine for ileal release, ileo-colonic release and/or colonic release can be formulated using conventional fluid-bed granulation procedures and equipment.

Example 37 is a capsule (or optionally tablet) formulation of oral buprenorphine for ileo-colonic and colonic delivery.

### Example 37

Modified Release Buprenorphine HCl Capsules
for Ileo-colonic or Colonic Delivery

| Ingredients | Qty./Unit |
|---|---|
| 8. Buprenorphine HCl | 12 mg |
| 9. HPMC 2208, USP | 150 mg |
| 10. Carnauba wax | 30 mg |
| 11. HPMC 2910, USP | 15 mg |
| 12. Magnesium Stearate | 2 mg |
| 13. Stearic acid | 8 mg |
| 14. Talc | 3 mg |

In Example 37, place the ingredients 1, 2 and 3 in the granulator and mix for 15 minutes. Dissolve ingredient 4 in water (mix in how water, then cool down) and spray into the fluidized mixture. Dry to approximately 5% moisture. Sequentially add ingredient 5, 6 and 7, with mixing steps between each addition. Encapsulate the dosage form in HPMC or other suitable capsules or compress into a tablet.

For the capsule formulation, prepare a composition of aqueous Eudragit® FS30D dispersion to coat 1.3 kg HPMC capsules: Eudragit FS30D 1207 g (solids, 362 g), Triethyl citrate 18 g (solids 18 g), Glyceryl monostearate 11 g (solids 11 g), Tween 80 (33%) 13 g (solids 4 g) and Distilled Water 728 g. Spray the dispersion onto the HPMC capsules using an Accela-Cota 10. The temperature of the capsule bed during the coating process is maintained between 26 and 32° C. The mean amounts of polymer applied ranges from 5 mg/cm² to 20 mg/cm², preferably 6 mg/cm² to 10 mg/cm².

For the tablet formulation, the composition of aqueous Eudragit® FS30D dispersion described above may be applied with a typical coating thicknesses of 10 to 30 mg polymer per cm² of tablet surface, preferably, 10 to 18 mg polymer per cm² capsule surface.

The in vitro performance of the dosage form can be tested using methods know in the art and described herein. When the capsules are tested using the USP Paddle Method in 0.1N HCl (pH 1.2) for two hours, followed by a switch to phosphate buffer pH 6.8 for one to two hours and then again, a switch to phosphate buffer pH 7.4, the capsules will remain intact and substantially non-releasing at pH 1.2 and pH 6.8 and provide substantial release at pH 7.4.

In some embodiments, oral modified release capsule of buprenorphine for ileo-colonic and colonic delivery in sustained release form can be made, as shown in Example 9 to 21.

Example 38 to 51 may be prepared as follows: (i) Dispense the specified hydrophobic controlled release material (e.g., hydrogenated Type I vegetable oil, hydrogenated Type II vegetable oil, polyoxyethylene stearates, polyoxyethylene distearates, glycerol monostearate, poorly water soluble, or high melting point waxes) into a mixer; (ii) Heat until fully melted; (iii) dispense the hydroxypropyl methyl cellulose (HPMC) into the mixer; (iv) Mix until dispersed; (v) Dispense the Aerosil into the same vessel; (vi) Mix until dispersed; (vii) Dispense the buprenorphine into the same vessel; (viii) Stir thoroughly with a high shear mixer; (ix) Transfer the mix into a liquid filling machine; (x) Fill into hard gelatin (or HPMC) capsule; (xi) Optionally, transfer the capsules to a banding machine and band the capsules. Optionally, cure the capsules by setting them at room temperature for a period of 1 to 7 days.

For Example 38 to 51, prepare a composition of aqueous Eudragit® FS30D dispersion to coat 1.3 kg HPMC capsules: Eudragit FS30D 1207 g (solids, 362 g), Triethyl citrate 18 g (solids 18 g), Glyceryl monostearate 11 g (solids 11 g), Tween 80 (33%) 13 g (solids 4 g) and Distilled Water 728 g. Spray the dispersion onto the HPMC capsules using an Accela-Cota 10. The temperature of the capsule bed during the coating process is maintained between 26 and 32° C. The mean amounts of polymer applied ranges from 5 mg/cm² to 20 mg/cm², preferably 6 mg/cm² to 15 mg/cm².

The in vitro performance of the dosage form can be tested using methods know in the art and described herein. When the capsules are tested using the USP Paddle Method in 0.1N HCl (pH 1.2) for two hours, followed by a switch to phosphate buffer pH 6.8 for one to two hours and then again, a switch to phosphate buffer pH 7.4, the capsules will remain intact and substantially non-releasing at pH 1.2 and pH 6.8, and they will release the buprenorphine at pH 7.4 over a prolonged period (e.g., over 12 to 48 hours).

### Example 38

Modified Release Buprenorphine HCl Capsules
for Slow Ileo-colonic or Colonic Release

| Ingredients | Quantity (mg)/Dose |
|---|---|
| Sterotex ® NF | 200 |
| Fractionated coconut oil | 70 |
| Methocel ® K 15M | 81 |
| Aerosil ® COK 84 | 4 |
| Buprenorphine HCl | 20 |

### Example 39

Modified Release Buprenorphine HCl Capsules
for Slow Ileo-colonic or Colonic Release

| Ingredients | Quantity (mg)/Dose |
|---|---|
| Beeswax | 200 |
| HPMC, K15M | 80 |
| Aerosil COK 84 | 8 |
| Buprenorphine HCl | 10 |

### Example 40

Modified Release Buprenorphine HCl Capsules
for Slow Ileo-colonic or Colonic Release

| Ingredients | Quantity (mg)/Dose |
|---|---|
| Sterotex NF | 150 |
| HPMC, K15M | 75 |
| Coconut oil | 75 |
| Aerosil COK 84 | 5 |
| Buprenorphine HCl | 12 |

### Example 41

Modified Release Buprenorphine HCl Capsules
for Slow Ileo-colonic or Colonic Release

| Ingredients | Quantity (mg)/Dose |
|---|---|
| Cithrol GMS | 275 |
| HPMC, K100M | 40 |
| Aerosil COK 84 | 10 |
| Buprenorphine HCl | 20 |

### Example 42

Modified Release Buprenorphine HCl Capsules
for Slow Ileo-colonic or Colonic Release

| Ingredients | Quantity (mg)/Dose |
|---|---|
| Hydrokote 112 | 250 |
| HPMC, K15M | 60 |
| Aerosil COK 84 | 10 |
| Buprenorphine HCl | 5 |

### Example 43

Modified Release Buprenorphine HCl Capsules
for Slow Ileo-colonic or Colonic Release

| Ingredients | Quantity (mg)/Dose |
|---|---|
| Beeswax | 200 |
| HPMC, Pharmacoat | 62.5 |
| 606 | |
| Aerosil COK 84 | 7.5 |
| Buprenorphine HCl | 25 |

### Example 44

Modified Release Buprenorphine HCl Capsules
for Slow Ileo-colonic or Colonic Release

| Ingredients | Quantity (mg)/Dose |
|---|---|
| Gelucire 50/02 | 190 |
| Methocel K 100M | 35 |
| Aerosil COK 84 | 10 |
| Buprenorphine HCl | 10 |

### Example 45

Modified Release Buprenorphine HCl Capsules
for Slow Ileo-colonic or Colonic Release

| Ingredients | Quantity (mg)/Dose |
|---|---|
| Cetyl alcohol | 280 |
| Methocel K 100M | 50 |
| Aerosil COK 84 | 10 |
| Buprenorphine HCl | 40 |

### Example 46

Modified Release Buprenorphine HCl Capsules
for Slow Ileo-colonic or Colonic Release

| Ingredients | Quantity (mg)/Dose |
|---|---|
| Sterotex NF | 320 |
| Methocel K 15M | 60 |
| Aerosil COK 84 | 10 |
| Buprenorphine HCl | 15 |

### Example 47

Modified Release Buprenorphine HCl Capsules
for Slow Ileo-colonic or Colonic Release

| Ingredients | Quantity (mg)/Dose |
|---|---|
| Cithrol GMS | 320 |
| Methocel K 100M | 55 |
| Aerosil COK 84 | 15 |
| Buprenorphine HCl | 15 |

### Example 48

Modified Release Buprenorphine HCl Capsules
for Slow Ileo-colonic or Colonic Release

| Ingredients | Quantity (mg)/Dose |
|---|---|
| Sterotex ® NF | 100 |
| Fractionated coconut oil | 70 |
| Beeswax | 100 |
| Methocel ® K 15M | 81 |
| Aerosil ® COK 84 | 4 |
| Buprenorphine HCl | 30 |

### Example 49

Modified Release Buprenorphine HCl Capsules
for Slow Ileo-colonic or Colonic Release

| Ingredients | Quantity (mg)/Dose |
|---|---|
| Cetyl alcohol | 270 |
| Methocel K 100M | 50 |
| Aerosil COK 84 | 10 |
| Buprenorphine HCl | 40 |

### Example 50

Modified Release Buprenorphine HCl Capsules
for Slow Ileo-colonic or Colonic Release

| Ingredients | Quantity (mg)/Dose |
|---|---|
| Sterotex NF | 293 |
| Methocel K 15M | 45 |
| Aerosil COK 84 | 10 |
| Buprenorphine HCl | 10 |

### Example 51

Modified Release Buprenorphine HCl Capsules
for Slow Ileo-colonic or Colonic Release

| Ingredients | Quantity (mg)/Dose |
|---|---|
| Cithrol GMS | 325 |
| Methocel K 100M | 55 |
| Aerosil COK 84 | 15 |
| Buprenorphine HCl | 20 |

Examples 52 and 53 are oral modified release tablets of buprenorphine for ileo-colonic and colonic delivery in sustained release.

The core tablet of buprenorphine is prepared using the composition shown in the Tables for Examples 52 and 53 using the following procedure: (i) Mix 1, 2, (and 3), and 5 and pass through #20 mesh; (ii) Dissolve 4 in 7; (iii) Granulate the powder from (i) using a fluid bed granulator and then dry; (iv) Pass the dry granules through #20 mesh; (v) Mix the granules with 6; (vi) Compress to tablet using plain/plain tooling.

Prepare a coating solution comprising: (a) Ethylcellulose (Ethocel PR100), 9.0 to 15 mg per tablet; (b) Polyvinylpyrrolidone (Kollidon 90F), 3.0 to 7.0 mg per tablet; (c) Dibutyl Sebacate, 2.0 to 4.0 mg per tablet; (d) Denatured Alcohol 150 to 300 mg per tablet (evaporates during process). Spray coating solution using coating pan until desired in vitro release is achieved as described herein. Adjust coating solution composition or coating weight/thickness as required. The coating weight is usually approximately 5 to 40% w/w.

For the initial trial, prepare one coating comprising: (a) Ethylcellulose (Ethocel PR100), 9.2 mg per tablet; (b) Polyvinylpyrrolidone (Kollidon 90F), 4.15 mg per tablet; (c) Dibutyl Sebacate, 2.70 mg per tablet; (d) Denatured Alcohol 170 mg per tablet (evaporates during process). Prepare another coating comprising: (a) Ethylcellulose (Ethocel PR100), 14.15 mg per tablet; (b) Polyvinylpyrrolidone (Kollidon 90F), 5.1 mg per tablet; (c) Dibutyl Sebacate, 3.85 mg per tablet; (d) Denatured Alcohol 245 mg per tablet (evaporates during process).

### Example 52

Modified Release Buprenorphine HCl Tablets
for Slow Ileo-colonic or Colonic Release

| | Quantity |
|---|---|
| Ingredients | (mg) |
| 1. Buprenorphine HCl | 40.00 |
| 2. Microcrystalline | 90.00 |
| Cellulose | |
| 3. HPMC (low viscosity | 46.00 |
| grade) | |
| 4. Polyvinyl Alcohol | 2.00 |
| 5. Colloidal Silicon | 1.00 |
| Dioxide | |
| 6. Sodium Stearyl | 1.00 |
| Fumarate | |
| 7. Purified Water * | As needed to form granules |

* Evaporated during process

### Example 53

Modified Release Buprenorphine HCl Tablets
for Slow Ileo-colonic or Colonic Release

| | Quantity |
|---|---|
| Ingredients | (mg) |
| 1. Buprenorphine HCl | 10.00 |
| 2. Microcrystalline | 90.00 |
| Cellulose | |
| 3. HPMC (low viscosity | — |
| grade) | |
| 4. Polyvinyl Alcohol | 2.00 |
| 5. Colloidal Silicon | 1.00 |
| Dioxide | |
| 6. Sodium Stearyl | 1.00 |
| Fumarate | |
| 7. Purified Water * | 41.60 |
| | (to form granules) |

* Evaporated during process

Alternatively, Examples 522 and 53 may be coated with a suitable amount of Eudragit® S100, or Eudragit® S 12.5, or Eudragit® FS 30D

### Example 54

Example 54 provides a method for manually sealing the overlapping region of the HPMC capsule body and cap using a process called banding. The banding solution is prepared as follows: (i) prepare the banding solution with 18 g of 4.5 cps HPMC along with 33 g of water and heated to 80° C. in a beaker; (ii) stir the mixture until the HPMC is well dispersed in the water; (iii) to completely solubilize the HPMC, add 50 mL of absolute alcohol and sonicate for 10 minutes; (iv) seal capsules using equipment such as the Lab band sealing machine (LBS 100) or the Qualicaps® Lab-Top Capsule Band-Sealer (S-1); (v) apply banding solution uniformly to the external edge of the gap of the capsule to be sealed to form a liquid ring around the circumference of the capsule and remove excess sealing liquid from the exterior of the capsule; (vi) dry the capsules by applying thermal energy, such as hot air and a controlled temperature oven. For larger scale banding, automated equipment such as the Qualicaps S-40 or S-100 capsule band-sealing machine or the Hermetica® is a capsule banding machine may be used to seal filled, two-piece capsules with a single or double band of substances such as gelatin or hypromellose at the joined portion of the cap and body.

### Example 55

Example 55 provides an alternative method of coating small batches of capsules to provide ileo-colonic or colonic deliver of the oral buprenorphine dosage form for rapid release or slow release (depending on the dosage form used). This method can be used in place of other described enteric coating methods. The method can be readily modified by those skilled in the art to coat tablet dosage forms. In this example, coating is carried out using 45% suspension of Eudragit® FS30D. Coating solution is prepared by homogenizing talc and triethyl citrate in water for 10 minutes. This suspension is poured into Eudragit FS 30 D dispersion under stirring. This spray suspension is transferred through 0.5 mm sieve. Coating suspension is spread by spray gun on a moving capsule bed. The process is carried out in conventional coating pan (see Table below). The coating solution may be further modified to achieve targeted GI delivery or release in or distal to the duodenum, jejunum, ileum, ileo-cecal junction, ileo-colonic region or colon, using methods known in the art and those described herein.

Composition of Coating Solution

| Ingredients | Enteric coating | |
|---|---|---|
| Eudragit FS 30 D | 150 | g |
| Talc | 22.5 | g |
| Triethyl citrate | 2.5 | g |
| Water | 175 | g |

The coating process parameters like the coating pan, baffles, inlet air temperature, product temperature, exhaust and spray air pressure are shown in Table below.

Coating Process Parameters

| Coating pan | 12 | inch |
|---|---|---|
| Baffles | Present | |
| Silicone tube od/id | 2 | mm |
| Inlet air temperature | 40 °C. | |
| Product Temperature | 30 °C. | |
| Exhaust | ON | |
| Blower | ON | |
| Spray air pressure | 2 | bar |

### Example 56

Example 56 provides an alternative method of coating small batches of capsules to provide ileo-colonic or colonic delivery of the oral buprenorphine dosage form for rapid release or slow release (depending on the dosage form used). This method can be used in place of other described enteric coating methods. The method can be readily modified by those skilled in the art to coat tablet dosage forms. The coating solution may be further modified to achieve targeted GI delivery or release in or distal to the duodenum, jejunum, ileum, ileo-cecal junction, ileo-colonic region or colon, using methods known in the art and those described herein. An initial coating solution is provided in the Table below. This can be modified further to achieve the desired delivery profile:

| S. No. | Ingredients | % w/w |
|---|---|---|
| 1 | Eudragit S 100 | 6.1 |
| 2 | Triethyl citrate | 0.9 |
| 3 | Talc | 3.0 |
| 4 | Isopropyl alcohol | 85.0 |
| 5 | Water | 5.0 |
| | | 100.0 |

The procedure for preparation of the above coating solution is: (a) dissolve Eudragit in 70% of total IPA and 100% of water; (b) homogenize talc and triethyl citrate in 30% of remaining IPA; (c) add talc and triethyl citrate dispersion to Eudragit solution and stir gently. Apply the coat by dip coating using the ProCoater® (Torpac, N.J.), with the method recommended by the manufacturer or preferably described by described by Dodds and Podczeck (Tablets & Capsules, January 2008, CSC Publishing Inc). Once dry, check weight gain. Additional coating may be applied to provide further weight gain. Batches with polymer target weight gains of about 10 mg/cm² to about 20 mg/cm² are prepared (for example, 10 mg/cm² or 15 mg/cm² and 20 mg/cm²). The approximate surface area of Capsule Size 00, 0, 1 and 2 are 6.16 cm², 5.07 cm2, 4.06 cm2, and 3.43 cm2, respectively (see Jones, Tablets & Capsules, January 2009 and April 2009, CSC Publishing Inc). This provides a target weight gain range shown below:

| Capsule | | | | |
|---|---|---|---|---|
| | Surface | Total Capsule Weight Gain | | |
| Size | Area | 10 mg/cm² | 15 mg/cm² | 20 mg/cm² |
| 00 | 6.16 cm² | 60 mg | 90 mg | 120 mg |
| 0 | 5.07 cm² | 50 mg | 75 mg | 100 mg |
| 1 | 4.06 cm² | 40 mg | 60 mg | 80 mg |
| 2 | 3.43 cm² | 35 mg | 50 mg | 70 mg |

To determine if the desired target delivery or release has been achieved, a disintegration tester is used (e.g. manually with the ERWEKA® ZT 120, ZT 120, ZT 220, or ZT 320, or using the automated ZT 850). Alternatively, an orbital shaker or a beaker with an agitator (or magnetic stirrer at moderate or high speed) is used, first in 0.1N HCl for two hours, then with a change in media to water at pH>7 (or other suitable pH). The capsules shell remains intact in 0.1 N HCL (pH 1.2) for two hours and disintegrates or ruptures at the desired pH (e.g., pH 7) usually after 5 to 90 minutes (the higher the weight gain, the longer they stay intact at pH 7). For modified release dosage forms intended to provide targeted GI delivery (e.g., ileo-colonic or colonic delivery) for subsequent slow or sustained release, the capsule shell will rupture, disintegrate, or dissolve but the contents may remain intact (depending on the dosage form used).

A more specific and reliable method to determine coating integrity and attainment of target GI delivery (which does not rely on visual inspection of capsules) for both modified release forms which rapidly release drug and those which slowly release (sustained release) drug in the target GI environment is the use of a dissolution methods where the drug release is quantified. When such the capsules are tested using the USP Paddle Method in 0.1N HCl (pH 1.2) for 2 hours at 37° C., the capsules (or tablets) remain intact and substantially non-releasing. Following a switch to the USP Basket or Paddle Method at 100 rpm in 900 mL distilled water (time=0 hour begins here) at 37° C. at the target pH, the dosage form begins to provide rapid or slow release of the drug usually after 5 to 90 minutes.

The optimal coating solution composition, weight gain and process parameters to achieve the desired target delivery or release is achieved by simultaneously testing the coated dosage form dosage form at various pH using the USP Paddle Method in 0.1N HCl (pH 1.2) for 2 hours at 37° C., followed by a switch to the USP Basket or Paddle Method at 100 rpm in 900 mL distilled water (time=0 hour begins here) at 37° C. at the target pH (e.g., pH 6.5, pH 6.8, pH 7.0, pH 7.5). For modified release dosage forms intended to provide targeted GI delivery for rapid release of buprenorphine, less than 10% of drug should be released at pH 1.2.

Various alternative small and large scale methods of organic and aqueous coating of tablets and capsules have been described or referenced herein, or are known those skilled in the art. Enteric coatings may also be applied over tablet and capsule osmotic delivery dosage forms of oral buprenorphine, including push pull osmotic pumps, monolithic osmotic delivery systems and controlled porosity osmotic pumps to provide targeted delivery in the GI tract.

### Example 57

A push-pull osmotic pump (PPOP) dosage form of oral buprenorphine was prepared using the method described below. Ingredients 1, 2 and 3 of drug layer were sifted (through BSS 22) dry mixed, and granulated with IPA. The granules, after drying and sifting (through BSS 22) were lubricated with ingredients 4 to 6 (passed through 30 BSS) of drug layer blend. Similarly, ingredients 1, 2, 3 and 4 of push layer were granulated with IPA. The granules, after drying and sifting were lubricated with ingredients 5 to 7. Bilayer tablets were compressed on a single punch compression machine using 8 mm punch. First the push layer was filled into die cavity and pressed lightly followed by addition of drug layer and mass is compressed to obtain tablets having minimum hardness of 4.5 kg/cm². A 4% w/v solution containing cellulose acetate and PEG 4000 in ratio of 90:10 was used for the coating of the bilayer tablets. Cellulose acetate and PEG 4000 were dissolved in acetone, stirred and sonicated to obtain clear transparent solution. Coating was done till the weight gain of 20-22% w/w which takes around 2.5 hrs. Coated tablets were dried in the oven for 16 to 20 hours; 55° C. Coated tablets were drilled using 8.0 mm drill at the centre of tablet on the drug layer side to create an appropriate orifice.

Modified Release Osmotic Dosage Form Drug Layer

| | Drug Layer | mg/Tablet | % w/w |
|---|---|---|---|
| 1 | Buprenorphine HCl | 10 | 6.45 |
| 2 | Polyox WSR N 80 | 136 | 87.74 |
| 3 | HPMC E5 | 7 | 4.51 |
| 4 | Magnesium Stearate | 1 | 0.65 |
| 5 | Colloidal Silicon Dioxide | 0.5 | 0.32 |
| 6 | Talc | 0.5 | 0.32 |

Modified Release Osmotic Dosage Form Push Layer

| | Push Layer | mg/Tablet | % w/w |
|---|---|---|---|
| 1 | Polyethylene oxide (Polyox WSR | 63 | 63 |
| | Coagulant) | | |
| 2 | Sodium Chloride | 29 | 29 |
| 3 | HPMC E5 | 5 | 5 |
| 4 | Red Ferric Oxide | 1 | 1 |
| 5 | Magnesium Stearate | 0.8 | 0.8 |
| 6 | Colloidal Silicon Dioxide | 0.7 | 0.7 |
| 7 | Talc | 0.5 | 0.5 |

The tablet dissolution was evaluated in 100 mL of pH 6.8 phosphate buffer using the USP paddle method at 50 rpm. See Table below.

Modified Release Osmotic Dosage Form Dissolution

| Time | Dissolution Rate |
|---|---|
| (hr) | (% w/w) |
| 0 | 0 |
| 1 | 27.0 |
| 2 | 29.0 |
| 4 | 47.5 |
| 6 | 55.6 |
| 9 | 70.0 |
| 12 | 77.0 |
| 14 | 80.1 |
| 16 | 83.2 |
| 18 | 83.5 |
| 20 | 84.7 |
| 24 | 85.1 |
| Crush* | 97.3 |
| Drug Content Assay | 99.57% w/w |

### Example 58

A push-pull osmotic pump (PPOP) dosage form of oral buprenorphine was prepared using the method described below. Ingredients 1, 2 and 3 of drug layer were sifted (through BSS 22) dry mixed, and granulated with IPA. The granules, after drying and sifting (through BSS 22) were lubricated with ingredients 4 to 6 (passed through 30 BSS) of drug layer blend. Similarly, ingredients 1, 2, 3 and 4 of push layer were granulated with IPA. The granules, after drying and sifting were lubricated with ingredients 5 to 7. Bilayer tablets were compressed on a single punch compression machine using 8 mm punch. First the push layer was filled into die cavity and pressed lightly followed by addition of drug layer and mass is compressed to obtain tablets having minimum hardness of 7.5 kg/cm². A 4% w/v solution containing cellulose acetate and PEG 4000 in ratio of 95:5 was used for the coating of the bilayer tablets. Cellulose acetate and PEG 4000 were dissolved in acetone, stirred and sonicated to obtain clear transparent solution. Coating was done till the weight gain of 20-22% w/w which takes around 2.5 hrs. Coated tablets were drilled using 8.0 mm drill at the centre of tablet on the drug layer side to create an appropriate orifice.

Modified Release Osmotic Dosage Form Drug Layer

| | Drug Layer | mg/Tablet | % w/w |
|---|---|---|---|
| 1 | Buprenorphine HCl* | 10 | 6.45 |
| 2 | (Polyox WSR N 80) | 136 | 87.74 |
| 3 | HPMC E5 | 7 | 4.51 |
| 4 | Magnesium stearate | 1 | 0.65 |
| 5 | Colloidal silicon dioxide | 0.5 | 0.32 |
| 6 | Talc | 0.5 | 0.32 |

Modified Release Osmotic Dosage Form Push Layer

| | Push Layer | mg/Tablet | % w/w |
|---|---|---|---|
| 1 | Polyethylene oxide (Polyox WSR | 63 | 63 |
| | Coagulant) | | |
| 2 | Sodium Chloride | 29 | 29 |
| 3 | HPMC E5 | 5 | 5 |
| 4 | Red Ferric Oxide | 1 | 1 |
| 5 | Magnesium Stearate | 0.8 | 0.8 |
| 6 | Colloidal Silicon Dioxide | 0.7 | 0.7 |
| 7 | Talc | 0.5 | 0.5 |

The tablet dissolution was evaluated in 100 mL of pH 6.8 phosphate buffer using the USP paddle method at 50 rpm. See Table below.

Modified Release Osmotic Dosage Form Dissolution

| Time | Dissolution Rate |
|---|---|
| (hr) | (% w/w) |
| 0 | 0 |
| 1 | 0.66 |
| 2 | 3.34 |
| 4 | 8.21 |
| 6 | 13.70 |
| 8 | 17.58 |
| 10 | 25.67 |
| 12 | 41.10 |
| 16 | 60.33 |
| 20 | 80.68 |
| 24 | 95.95 |
| Crush* | 101.4 |
| Drug Content Assay | 101.94% w/w |

### Example 59

A push-pull osmotic pump (PPOP) dosage form of oral buprenorphine was prepared using the method described below. To assess the effects of acid on dissolution, dissolution was assessed at pH 1.2 and pH 6.8. The dosage was showed robust and comparable dissolution at pH 1.2 and pH 6.8. Ingredients 1, 2 and 3 of drug layer were sifted (through BSS 22) dry mixed, and granulated with IPA. The granules, after drying and sifting (through BSS 22) were lubricated with ingredients 4 to 6 (passed through 30 BSS) of drug layer blend. Similarly, ingredients 1, 2, 3 and 4 of push layer were granulated with IPA. The granules, after drying and sifting were lubricated with ingredients 5 to 7. Bilayer tablets were compressed on a single punch compression machine using 8 mm punch. First the push layer was filled into die cavity and pressed lightly followed by addition of drug layer and mass is compressed to obtain tablets having minimum hardness of 6.5 kg/cm². A 4% w/v solution containing cellulose acetate and PEG 4000 in ratio of 90:10 was used for the coating of the bilayer tablets. Cellulose acetate and PEG 4000 were dissolved in acetone, stirred and sonicated to obtain clear transparent solution. Coating was done till the weight gain of 20-22% w/w which takes around 2.5 hrs. Coated tablets were drilled using 8.0 mm drill at the centre of tablet on the drug layer side to create an appropriate orifice.

Modified Release Osmotic Dosage Form Drug Layer

| | Drug Layer | mg/Tablet | % w/w |
|---|---|---|---|
| 1 | Buprenorphine HCl* | 10 | 6.45 |
| 2 | (Polyox WSR N 80) | 136 | 87.74 |
| 3 | HPMC E5 | 7 | 4.51 |
| 4 | Magnesium stearate | 1 | 0.65 |
| 5 | Colloidal silicon dioxide | 0.5 | 0.32 |
| 6 | Talc | 0.5 | 0.32 |

Modified Release Osmotic Dosage Form Push Layer

| | Push Layer | mg/Tablet | % w/w |
|---|---|---|---|
| 1 | Polyethylene oxide (Polyox WSR | 63 | 63 |
| | Coagulant) | | |
| 2 | Sodium Chloride | 29 | 29 |
| 3 | HPMC E5 | 5 | 5 |
| 4 | Red Ferric Oxide | 1 | 1 |
| 5 | Magnesium Stearate | 0.8 | 0.8 |
| 6 | Colloidal Silicon Dioxide | 0.7 | 0.7 |
| 7 | Talc | 0.5 | 0.5 |

The tablet dissolution was evaluated in (i) 100 mL of pH 6.8 phosphate buffer and in and (ii) at pH 1.2 for 2 hrs, followed by pH 6.8 phosphate buffer for the remaining time using the USP paddle method at 50 rpm. See Table below. The dosage form showed pH independent dissolution.

Modified Release Osmotic Dosage Form Dissolution

| Time | Dissolution Rate (i) | Dissolution Rate (ii) |
|---|---|---|
| (hr) | (% w/w) | (% w/w) |
| 0 | 0 | 0 |
| 1 | 1.23 | 2.25 |
| 2 | 8.29 | 8.80 |
| 4 | 18.94 | 18.34 |
| 6 | 31.54 | 39.39 |
| 8 | 63.15 | 58.27 |
| 24 | 91.12 | 93.90 |
| Crush* | 103.10 | 104.02 |
| Drug Content Assay | 102.58 | 102.28 |

### Example 60

A matrix dosage form of oral buprenorphine was prepared using the method described below. 1. Weighed accurately Buprenorphine HCl, HPMC E15 LV, HPMC K15 M CR, MCC, ascorbic acid, citric acid, sodium citrate, EDTA and colloidal silicon dioxide. 2. Sifted ingredients of step 1 using mesh ASTM #40. 3. Blended step 2 ingredients for 10 minutes. 4. Weighed PVP K30 and alpha-tocopherol acetate and put in IPA and prepared binder solution. 5. Granulated step 3 blend using step 4 binder solution. 6. Dried step 5 granules in oven at 50° C. 7. Dried granules were sifted through mesh ASTM #25. 8. Weighed and sifted magnesium stearate through mesh #60. 9. Lubricated step 7 granules using pre sifted magnesium stearate for 5 minutes in a polybag. 10. Compressed the granules using 7.5 mm round standard concave punches.

| Components | mg/Tablet |
|---|---|
| Buprenorphine HCl (=to 10 | 10 |
| mg Buprenorphine Base) | |
| HPMC E 15 LV | 13.3 |
| HPMC K 15 M CR | 26.7 |
| MCC (Avicel ® PH 102) | 56.4 |
| Ascorbic Acid | 2.5 |
| Alpha-tocopherol Acetate | 0.5 |
| Citric Acid Anhydrous | 4 |
| Sodium Citrate Trihydrate | 4 |
| PVP K30 | 3 |
| EDTA | 0.1 |
| Aerosil ® | 3 |
| Magnesium stearate | 1.5 |
| Isopropyl Alcohol | qs |

The tablet dissolution was evaluated in (i) 100 mL of pH 6.8 phosphate buffer and in and (ii) in pH 1.2 (0.1 N HCl) for 24 hours using the USP paddle method at 50 rpm. See Table below. Despite exposure to pH 1.2 for 24 hours (unlike the recommended 2 hours at this pH to evaluate pH effects), the dosage form showed robust and pH independent dissolution.

Modified Release Osmotic Dosage Form Dissolution

| Time | Dissolution Rate (i) | Dissolution Rate (ii) |
|---|---|---|
| (hr) | (% w/w) | (% w/w) |
| 0 | 0.0 | 0.0 |
| 1 | 14.6 | 15.2 |
| 2 | 21.8 | 24.1 |
| 4 | 35.6 | 39.4 |
| 6 | 46.3 | 55.3 |
| 8 | 57.4 | 64.6 |
| 12 | 73.3 | 80.3 |
| 16 | 83.3 | 90.9 |
| 20 | 89.6 | 94.8 |
| 24 | 94.8 | 93.7 |
| Recovery after 24 | 102.0 | 93.3 |
| hrs | | |

Antinociceptive Effects of Oral Buprenorphine

Radiant Heat Tail Flick Test

Background

The tail flick test was first described by D'Amour and Smith (1941), and remains essentially unchanged in application. (See generally D'Amour, F. E. and Smith, D. L., “A method for determining loss of pain sensation”, J. Pharmacol. Exp. Therap., 72:74-79 (1941); Dewey, D. L. and Harris, L. S., The Tail-flick test. In: S. Ehrenpreis and A. Neidle (Eds.), Methods in Narcotic Research, Marcel Dekker, Inc., New York, 1975, pp. 101-109; and Dubner, R. and Ren, K., “Assessing transient and persistent pain in animals.” In: P. D. Wall and R. Melzack (Eds.), Textbook of Pain, Churchill Livingstone, London, 1999, pp. 359-369). Quite simply, the tail of a rat or mouse is exposed to radiant heat, and the latency to withdraw is determined. The basal heat intensity is set so that naïve rats withdraw their tails within 2 to 3 sec. A cut-off latency of 10 sec (i.e., 3 to 4 times basal control value) is commonly employed to prevent tissue damage. An alternative to using radiant heat is to dip the tail into a water bath maintained at a fixed temperature, usually in the moderately noxious range of about 52° C. or 55° C. One advantage of a water bath is that the temperature is kept constant.

The tail-flick test is considered to be very robust in that weak analgesic agents are not detected by this test. In contrast, it is considered highly selective. There is a high degree of correlation between drugs that are identified as antinociceptive in the tail-flick test and clinically active analgesic agents. Importantly, agents that are sedating and may produce a positive response in the writhing test or hot plate test do not show antinociceptive activity in the tail-flick test. It is even possible to perform the tail-flick test in lightly anesthetized animals.

### Example 61

Aim: The aim of this study was to evaluate the analgesic effect of the oral administration of buprenorphine in rats using the tail flick test of nociception. Dose-response relationships of orally administered buprenorphine were established in order to calculate the ED₅₀ value. The primary measurements were made using the tail flick test designed to detect effects of the treatment(s) on nociception in rats.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design: Three groups were employed in the present study with each group comprising of 8 animals. Each rat was administered oral buprenorphine 0.1 mg/kg, 0.3 mg/kg, or 1.0 mg/kg, following which the nociceptive threshold assessment was made. Tail flick test was performed 0, 15, 30, and 60 minutes after administration of the buprenorphine. Area under the curve was calculated using a graph between % MPE and time. Area under the curve was employed as a quantitative measure of the analgesic effect of the test drugs.

Measurement of Effect: Nociceptive threshold was measured with the tail flick test (D'Amour and Smith, 1941). The tail flick latency was considered as the time between exposure of the tail to radiant heat and tail withdrawal. Electrically heated nichrome wire was used as a source of radiant heat in the analgesiometer. The intensity of radiant heat was regulated in order to obtain a pretreatment latency between 2 and 4 sec in the animals. A cut-off latency time was fixed at 10 sec. Tail flick latency is expressed as a percentage of the maximum possible effect (MPE):

$$M P E ( \% ) = \frac{( Post treatment latency - pre treatment latency )}{( Cut \text{-} off time - pre treatment latency )} \times 100$$

Data Analysis: The anti-nociceptive effect at a particular dose level was assessed in terms of area under the curve calculated from a graph having time (relative to dosing time point) on the x-axis and percent maximum possible effect (% MPE) on the y-axis. Then a dose response curve was obtained from a graph having log dose(s) levels tested on the x-axis and their respective area under the curve on the y-axis. Then the dose response curve thus obtained was used to further calculate the ED₅₀ value of the test drug for the respective behavioral assessment method(s).

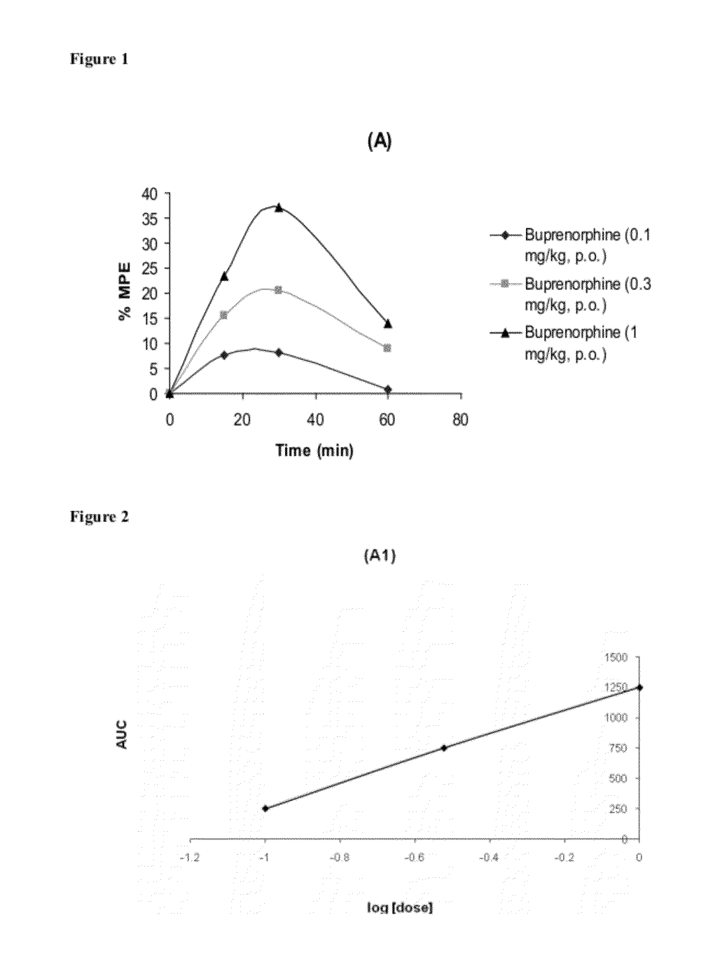

Results: Oral administration of buprenorphine caused a significant increase in the tail flick latency in rats in comparison to the control readings. This effect of buprenorphine was observed to be dependent of the dose. Thus, buprenorphine demonstrated a marked dose dependent analgesic effect in rats as also assessed in terms of its ED₅₀. The ED₅₀ values of orally administered buprenorphine as assessed using the tail flick test was 0.24 mg/kg. The peak time of the analgesic effect of oral buprenorphine was found to be about 30 minutes post-treatment (see FIGS. 1 and 2).

### Example 62

Aim: The aim of this study was to evaluate the analgesic effect of orally administered buprenorphine following administration into different parts of the gastrointestinal tract in transiently ether anaesthetized rats, using the tail flick test of nociception. The primary measurements were made using the tail flick test designed to detect effects of the treatment(s) on nociception in rats.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design: Three groups were employed in the present study with each group comprising of 8 animals. Each animal was subjected to transient ether anesthesia followed by a minor incision in the abdominal cavity through which buprenorphine 0.2 mg/kg was administered using a syringe into the stomach, ileum and colon. Tail flick test was performed 0, 30, 45 and 60 minutes after administration of the buprenorphine. Area under the curve was calculated using a graph between % MPE and time. Area under the curve was employed as a quantitative measure of the analgesic effect of the treatments.

Measurement of Effect: Nociceptive threshold was measured with the tail flick test (D'Amour and Smith, 1941). The tail flick latency was considered as the time between exposure of the tail to radiant heat and tail withdrawal. Electrically heated nichrome wire was used as a source of radiant heat in the analgesiometer. The intensity of radiant heat was regulated in order to obtain a pretreatment latency between 2 and 4 sec in the animals. A cut-off latency time was fixed at 10 sec. Tail flick latency is expressed as a percentage of the maximum possible effect (MPE):

$$M P E ( \% ) = \frac{( Post treatment latency - pre treatment latency )}{( Cut \text{-} off time - pre treatment latency )} \times 100$$

Data Analysis: The anti-nociceptive effect following intragastric, intra-ileal and intra-colonic administration was assessed in terms of area under the curve calculated from a graph having time (relative to dosing time point) on the x-axis and percent maximum possible effect (% MPE) on the y-axis. The results were expressed as mean±standard error of means (S.E.M.). Statistical analysis for was done using one-way ANOVA, followed by Tukey's multiple range tests as post-hoc analysis. A value of P<0.05 was considered to be statistically significant.

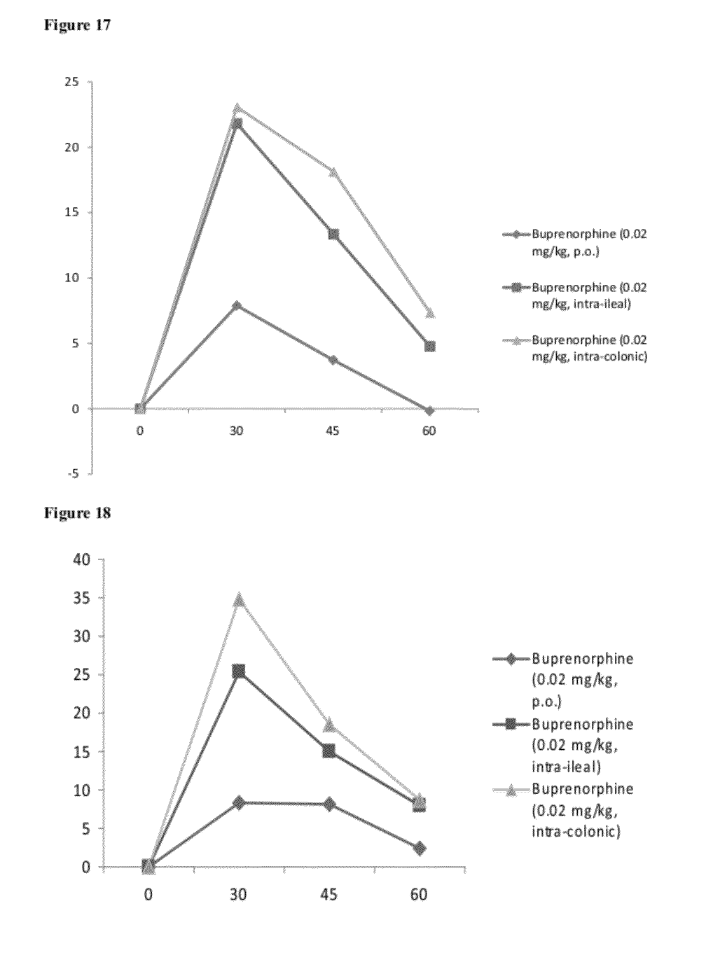

Results: Intra-gastric, intra-ileal, and intra-colonic administration of buprenorphine each caused a significant increase in the tail flick latency in rats. However, intra-gastric administration of buprenorphine produced only a modest analgesic effect, while both intra-ileal administration and intra-colonic administration of buprenorphine produced a robust analgesic response. The analgesic effects after intra-ileal and intra-colonic administration were significantly greater than those following intra-gastric administration of buprenorphine (see FIG. 17).

Hot Plate Test

Background

Heat is often used as a noxious stimulus in models of pain. Typically, the latency of the rat's response to the thermal stimulus is recorded as the dependent variable. To determine whether a test compound has analgesic properties, the latency responses of treated and control rats are evaluated. A significant increase in the latency to respond to the thermal stimulus after a treatment relative to a control treatment is interpreted as an antinociceptive or analgesic response. The latency of response will vary depending on the type of heat stimulus used. In the hot-plate assay, the rat is placed on a heated surface and the latency for the rat to respond to the stimulus, by licking its paw or jumping, is recorded.

### Example 63

Aim: The aim of this study was to evaluate the analgesic effect of the oral administration of buprenorphine in rats using the hot plate test of nociception. Dose-response relationships of orally administered buprenorphine were established in order to calculate the ED₅₀ value. The primary measurements were made using the hot plate test designed to detect effects of the treatment(s) on nociception in rats.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design: Three groups were employed in the present study with each group comprising of 8 animals. Each rat was administered oral buprenorphine 0.1 mg/kg, 0.3 mg/kg, or 1.0 mg/kg, following which the nociceptive threshold assessment was made. The hot plate test was performed 0, 15, 30, and 60 minutes after administration of the buprenorphine. Area under the curve was calculated using a graph between % MPE and time. Area under the curve was employed as a quantitative measure of the analgesic effect of the test drugs.

Measurement of Effect: Nociceptive threshold was measured with the Eddy's hot plate test. The reaction time was considered as the time between placing of the rat on the Eddy's hot plate heated to a temperature of 52.5° C. and the reaction of the animal in the form of a jump or fore paw licking reflex. A cut-off latency time was fixed at 10 sec. Reaction time is expressed as a percentage of the maximum possible effect (MPE):

$$M P E ( \% ) = \frac{( Post treatment latency - pre treatment latency )}{( Cut \text{-} off time - pre treatment latency )} \times 100$$

Data Analysis: The anti-nociceptive effect at a particular dose level was assessed in terms of area under the curve calculated from a graph having time (relative to dosing time point) on the x-axis and percent maximum possible effect (% MPE) on the y-axis. Then a dose response curve was obtained from a graph having log dose(s) levels tested on the x-axis and their respective area under the curve on the y-axis. Then the dose response curve thus obtained was used to further calculate the ED₅₀ value of the test drug for the respective behavioral assessment method(s).

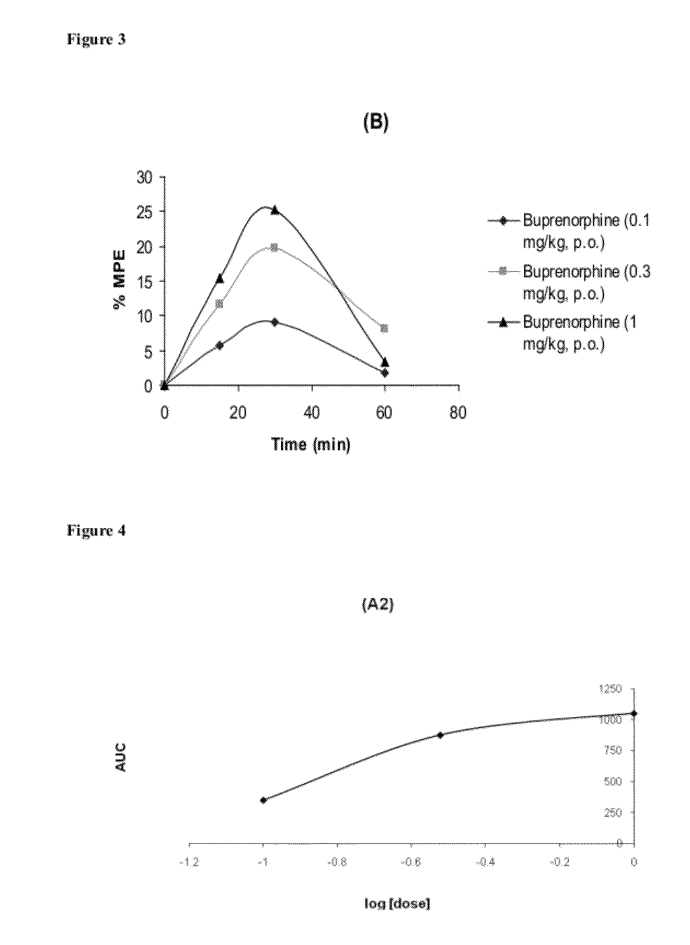

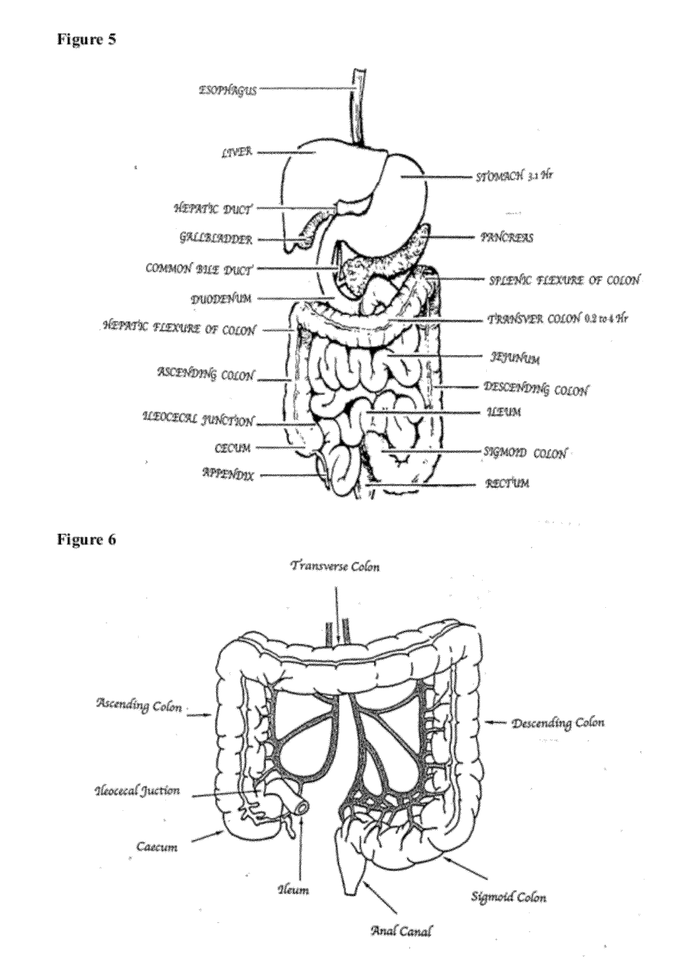

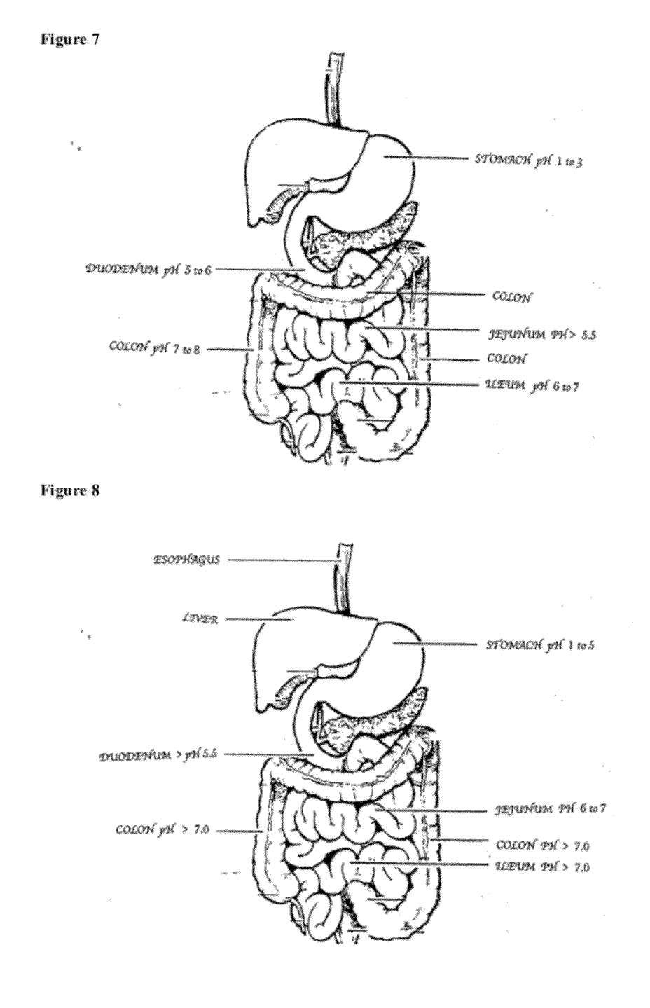

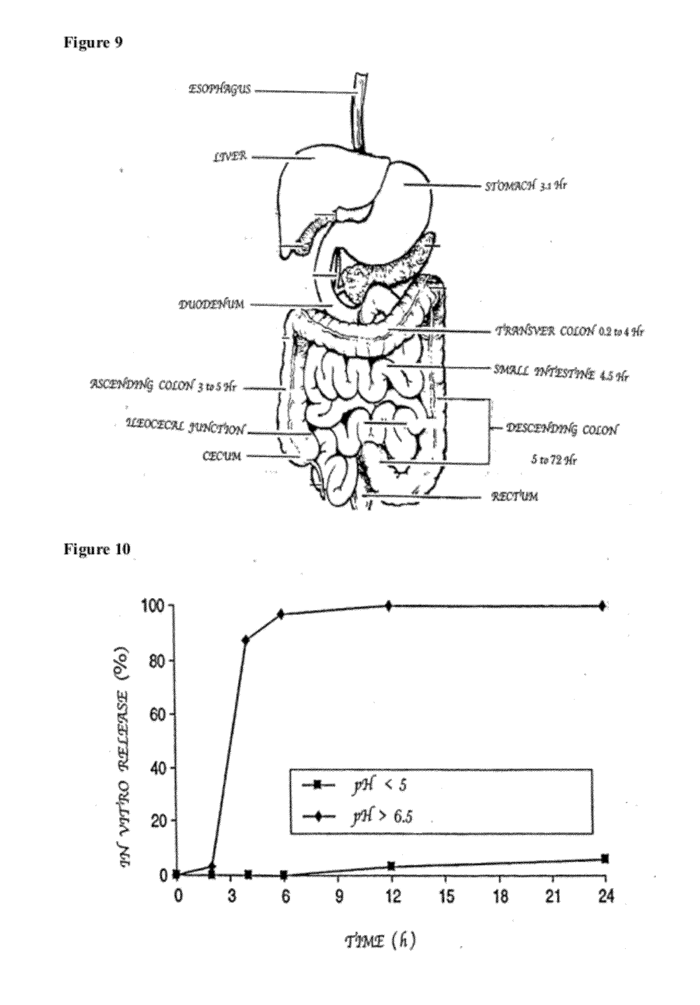

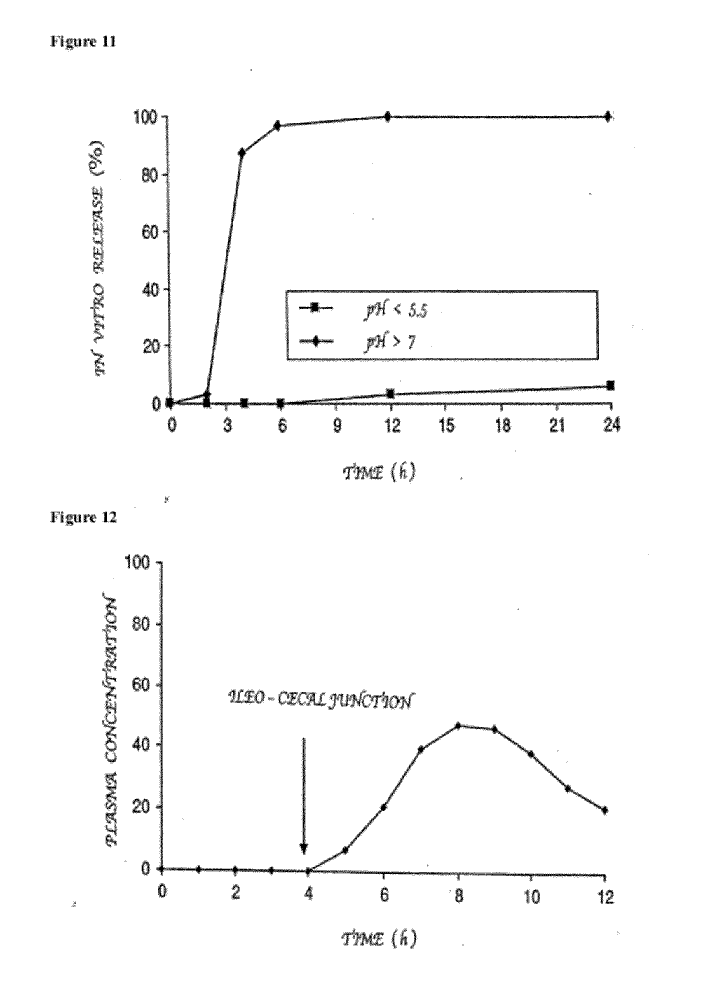

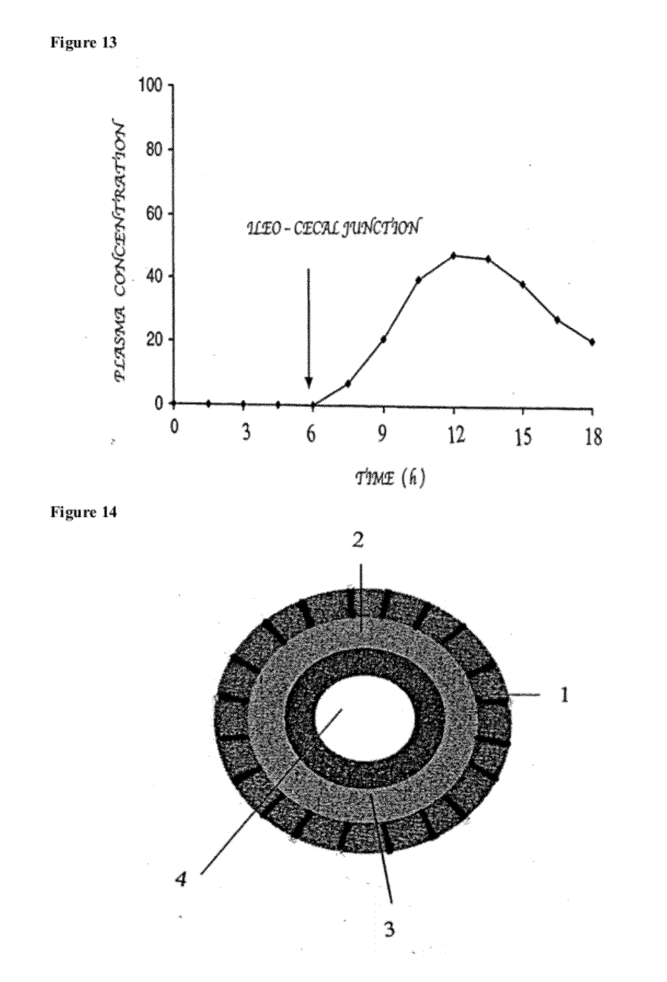

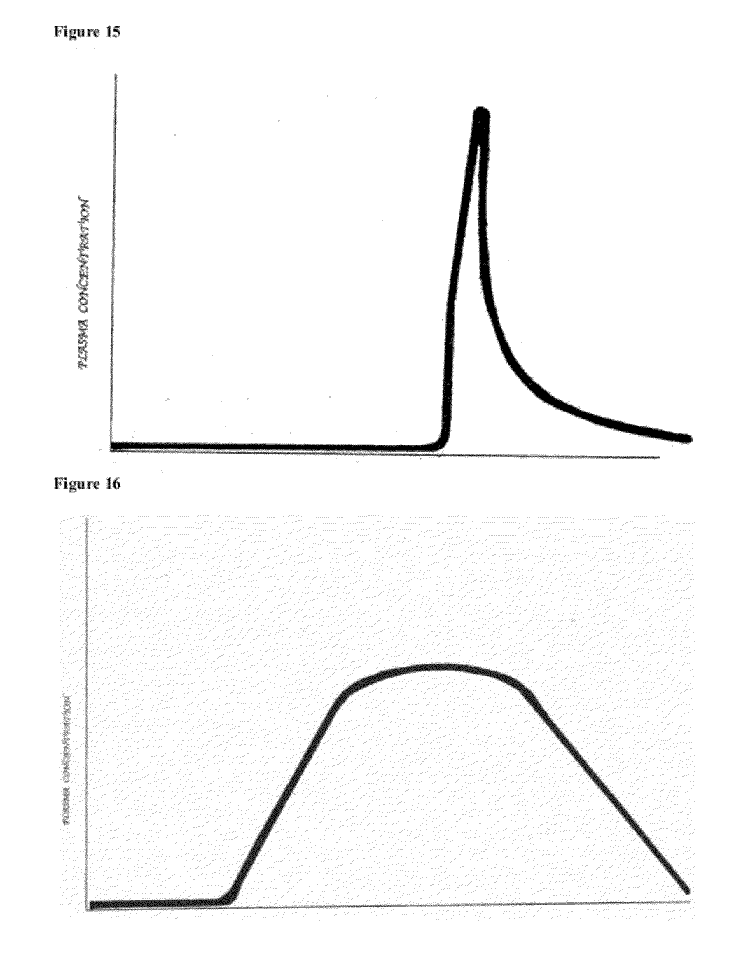

Results: Oral administration of buprenorphine caused a significant increase in the hot plate latency in rats in comparison to the control readings. This effect of buprenorphine was observed to be dependent of the dose. Thus, buprenorphine demonstrated a marked dose dependent analgesic effect in rats as also assessed in terms of its ED₅₀. The ED₅₀ values of orally administered buprenorphine as assessed using the hot plate test was 0.16 mg/kg. The peak time of the analgesic effect of oral buprenorphine was found to be about 30 minutes post-treatment (see FIGS. 3 and 4).

### Example 64

Aim: The aim of this study was to evaluate the analgesic effect of orally administered buprenorphine following administration into different parts of the gastrointestinal tract in transiently ether anaesthetized rats, using the hot plate test of nociception The primary measurements were made using the hot plate test designed to detect effects of the treatment(s) on nociception in rats.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design: Three groups were employed in the present study with each group comprising of 8 animals. Each animal was subjected to transient ether anesthesia followed by a minor incision in the abdominal cavity through which buprenorphine 0.2 mg/kg was administered using a syringe into the stomach, ileum and colon. Hot plate latency was assessed at 0, 30, 45 and 60 minutes after administration of the buprenorphine. Area under the curve was calculated using a graph between % MPE and time. Area under the curve was employed as a quantitative measure of the analgesic effect of the treatments.

Measurement of Effect: Nociceptive threshold was measured with the Eddy's hot plate test. The reaction time was considered as the time between placing of the rat on the Eddy's hot plate heated to a temperature of 52.5° C. and the reaction of the animal in the form of a jump or fore paw licking reflex. A cut-off latency time was fixed at 10 sec. Reaction time is expressed as a percentage of the maximum possible effect (MPE):

$$M P E ( \% ) = \frac{( Post treatment latency - pre treatment latency )}{( Cut \text{-} off time - pre treatment latency )} \times 100$$

Data Analysis: The anti-nociceptive effect following intragastric, intra-ileal and intra-colonic administration was assessed in terms of area under the curve calculated from a graph having time (relative to dosing time point) on the x-axis and percent maximum possible effect (% MPE) on the y-axis. The results were expressed as mean±standard error of means (S.E.M.). Statistical analysis for was done using one-way ANOVA, followed by Tukey's multiple range tests as post-hoc analysis. A value of P<0.05 was considered to be statistically significant.

Results: Intra-gastric, intra-ileal, and intra-colonic administration of buprenorphine each caused a significant increase in the hot plate latency in rats. However, intra-gastric administration of buprenorphine produced only a modest analgesic effect, while both intra-ileal administration and intra-colonic administration of buprenorphine produced a robust analgesic response. The analgesic effects after intra-ileal and intra-colonic administration were significantly greater than those following intra-gastric administration of buprenorphine (see FIG. 18).

### Chemotherapy Induced Peripheral Neuropathy

### Example 65

Aim: The aim of this study was to evaluate the effect of oral administration of buprenorphine in rats with vincristine induced painful neuropathy. The primary measurements were made mechanical and thermal allodynia designed to detect effects of the treatment(s) on neuropathic pain.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design: The vincristine model was adapted from Authier et al (1999). Rats were injected on five alternate days (day 4, 6, 8, 10 & 12) with vincristine (200 μg/kg i.p.) using an injection volume of 2 ml/kg. Thus, the cumulative dose of vincristine was computed to be 1 mg/kg. Tests took place three days after the last injection and continued over the next three weeks. Five groups were employed in the present study with each group comprising of 8 animals: Group I (Vehicle control group): Vehicle (2 ml/kg, i.p.) administration was followed by the pain assessment; Group II (Oral buprenorphine treatment group-I): Buprenorphine (0.01 mg/kg, p.o.) administration was followed by the pain assessment; Group III (Oral buprenorphine treatment group-II): Buprenorphine (0.03 mg/kg, p.o.) administration was followed by the pain assessment; Group IV (Oral buprenorphine treatment group-III): Buprenorphine (0.1 mg/kg, p.o.) administration was followed by the pain assessment; Group V (Oral buprenorphine treatment group-IV): Buprenorphine (0.3 mg/kg, p.o.) administration was followed by the pain assessment.

Measurement of Effect

Assessment of Mechanical Allodynia

Mechano-allodynia was assessed using von Frey filaments. The filaments are nylon monofilaments of different diameters that exert defined levels of force to cause the hair to bend. Once a rat was calm, a series of von Frey filaments with roughly exponential incremental target force (0.008, 0.02, 0.04, 0.07, 0.16, 0.4, 0.6, 1, 1.4, 2, 4, 6, 8, 10, 15, 26, 60, 100, 180, 300 g) were applied to the bottom of the right paw, behind the front pads and lateral to the medial pads of the rat. The filament was presented perpendicular to the rat paw and applied with enough force so as to cause a slight bend. It was held steadily against the paw for a period of 5 s. A response was considered positive if, within the 5-s period, the rat withdrew from the stimulus or briskly withdrew immediately after the filament was removed. A negative response was the one in which the rat did not withdraw within the given time. Testing was initiated using middle filament (2 g). A negative response required the use of next filament with greater bending force. A positive response required the use of next filament with less bending force. Both hind paws were tested for allodynia and the average of the two was recorded as data. Results were expressed in grams and determined before and 30 and 60 min after the administration of the test drugs.

Assessment of Cold Allodynia

For the assessment of cold allodynia, which was stable for 3 weeks, the rats were placed on a wire mesh covered with a plastic dome and were allowed to habituate until the exploratory behavior diminished. Cold allodynia was measured as the number of foot withdrawal responses after application of stimuli to the plantar surface of the paw. A drop of acetone (10 μl) was gently applied to the heel of the animal with a micro-pipette. A brisk foot withdrawal response (shaking, tapping, or licking) after the spread of acetone over the plantar region of the paw was considered a sign of cold allodynia. Acetone was applied two times (once every 5 min) on the left paw, and the number of reactions (shaking, tapping, or licking) was counted during 30 sec. A cut-off number of reactions were fixed at 20 per trial (i.e. 40). The score was expressed as accumulated numbers of reactions over the trials and determined before and 30 and 60 min after administration of the test drugs. Each individual test was expressed as a percentage of maximum possible effect (% MPE):

$$M P E ( \% ) = \frac{( Post treatment f requency - pre treatment frequency )}{( Cut \text{-} off f requency - pre treatment frequency )} \times 100$$

The peak effect was seen at a time point 30 minutes after the drug administration. MPE (%) at 30 min time point was employed as a quantitative measure of the analgesic effect of the test drugs.

Data Analysis: The anti-nociceptive effect was plotted assessed having time (relative to dosing time point) on the x-axis and percent maximum possible effect (% MPE) on the y-axis. The results were expressed as mean±standard error of means (S.E.M.). Statistical analysis for was done using one-way ANOVA, followed by Tukey's multiple range tests as post-hoc analysis. A value of P<0.05 was considered to be statistically significant.

Results

Response to Mechanical Allodynia

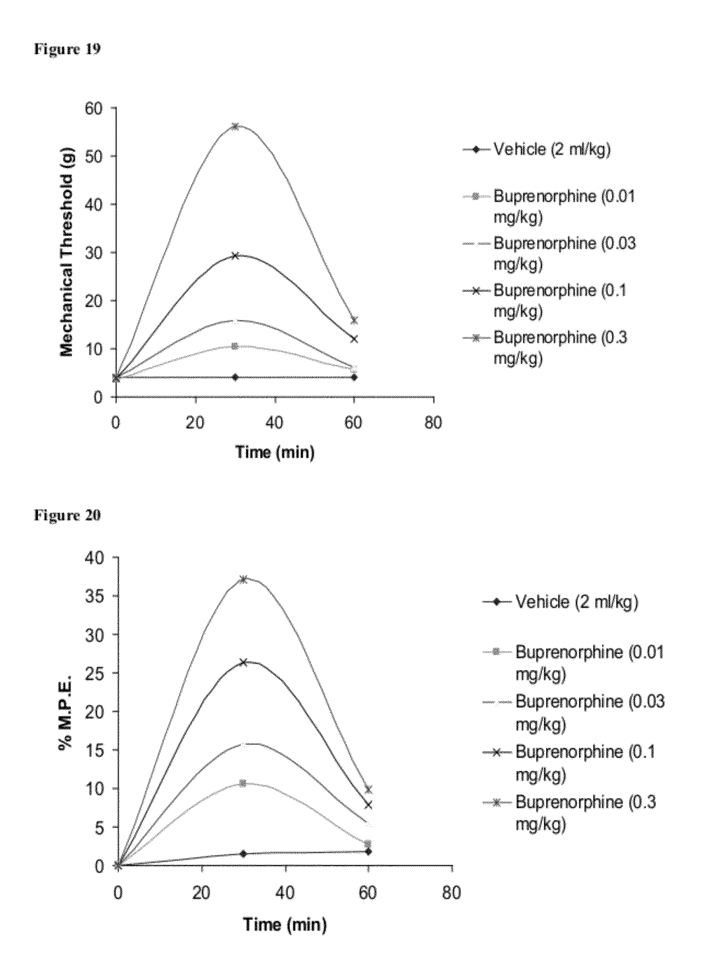

Baseline mean mechanical withdrawal threshold in the vincristine treatment induced neuropathic rat was observed to be 4.11+0.31 g. All animals included in the study had a mean threshold below five, thus conforming to the requirements of being considered neuropathic. Oral administration of buprenorphine demonstrated a dose dependent increase in the mechanical withdrawal threshold that elicits pain in the neuropathic rats. Moreover, the effect of the buprenorphine was dependent on the dose and the peak effect of drug was noted to be 30 min post-administration (FIG. 19).

Response to Cold Allodynia

Baseline of cold allodynia score was determined using the acetone drop test in the vincristine treatment induced neuropathic rats. Moreover, effect of the drug was dependent on the dose and the peak effect of drug was noted to be 30 min post-administration. Furthermore, the oral administration of buprenorphine demonstrated a dose dependent decrease in the cold allodynia score in the neuropathic rats (FIG. 20).

Conclusion: Oral administration of buprenorphine caused a significant increase in the mechanical allodynia withdrawal threshold and cold allodynia withdrawal threshold in rats with neuropathic pain in comparison to the control readings. This effect of buprenorphine was observed to be dependent of the dose. Thus, buprenorphine demonstrated a marked dose dependent effect in rats with vincristine induced neuropathy (see FIGS. 19 and 20).

### Example 66

Aim: The aim of this study was to evaluate the effect of oral administration of buprenorphine in rats with paclitaxel induced painful neuropathy. The primary measurements were made mechanical and thermal allodynia designed to detect effects of the treatment(s) on neuropathic pain.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design: Rats were injected on five alternate days (day 4, 6, 8, 10 & 12) with paclitaxel (2 mg/kg i.p.) using an injection volume of 2 ml/kg. Thus, the cumulative dose of paclitaxel was computed to be 10 mg/kg. Tests took place ten days after the last injection and continued over the next three weeks. Five groups were employed in the present study with each group comprising of 8 animals: Group I (Vehicle control group): Vehicle (2 ml/kg, i.p.) administration was followed by the pain assessment; Group II (Oral buprenorphine treatment group-I): Buprenorphine (0.01 mg/kg, p.o.) administration was followed by the pain assessment; Group III (Oral buprenorphine treatment group-II): Buprenorphine (0.03 mg/kg, p.o.) administration was followed by the pain assessment; Group IV (Oral buprenorphine treatment group-III): Buprenorphine (0.1 mg/kg, p.o.) administration was followed by the pain assessment; Group V (Oral buprenorphine treatment group-IV): Buprenorphine (0.3 mg/kg, p.o.) administration was followed by the pain assessment.

Measurement of Effect

Assessment of Mechanical Allodynia

Mechano-allodynia was assessed using von Frey filaments. The filaments are nylon monofilaments of different diameters that exert defined levels of force to cause the hair to bend. Once a rat was calm, a series of von Frey filaments with roughly exponential incremental target force (0.008, 0.02, 0.04, 0.07, 0.16, 0.4, 0.6, 1, 1.4, 2, 4, 6, 8, 10, 15, 26, 60, 100, 180, 300 g) were applied to the bottom of the right paw, behind the front pads and lateral to the medial pads of the rat. The filament was presented perpendicular to the rat paw and applied with enough force so as to cause a slight bend. It was held steadily against the paw for a period of 5 s. A response was considered positive if, within the 5-s period, the rat withdrew from the stimulus or briskly withdrew immediately after the filament was removed. A negative response was the one in which the rat did not withdraw within the given time. Testing was initiated using middle filament (2 g). A negative response required the use of next filament with greater bending force. A positive response required the use of next filament with less bending force. Both hind paws were tested for allodynia and the average of the two was recorded as data. Results were expressed in grams and determined before and 30 and 60 min after the administration of the test drugs.

Assessment of Cold Allodynia

For the assessment of cold allodynia, which was stable for 3 weeks, the rats were placed on a wire mesh covered with a plastic dome and were allowed to habituate until the exploratory behavior diminished. Cold allodynia was measured as the number of foot withdrawal responses after application of stimuli to the plantar surface of the paw. A drop of acetone (10 up was gently applied to the heel of the animal with a micro-pipette. A brisk foot withdrawal response (shaking, tapping, or licking) after the spread of acetone over the plantar region of the paw was considered a sign of cold allodynia. Acetone was applied two times (once every 5 min) on the left paw, and the number of reactions (shaking, tapping, or licking) was counted during 30 sec. A cut-off number of reactions were fixed at 20 per trial (i.e. 40). The score was expressed as accumulated numbers of reactions over the trials and determined before and 30 and 60 min after administration of the test drugs. Each individual test was expressed as a percentage of maximum possible effect (% MPE):

$$M P E ( \% ) = \frac{( Post treatment f requency - pre treatment frequency )}{( Cut \text{-} off f requency - pre treatment frequency )} \times 100$$

The peak effect was seen at a time point 30 minutes after the drug administration. MPE (%) at 30 min time point was employed as a quantitative measure of the analgesic effect of the test drugs.

Data Analysis: The anti-nociceptive effect was plotted assessed having time (relative to dosing time point) on the x-axis and percent maximum possible effect (% MPE) on the y-axis. The results were expressed as mean±standard error of means (S.E.M.). Statistical analysis for was done using one-way ANOVA, followed by Tukey's multiple range tests as post-hoc analysis. A value of P<0.05 was considered to be statistically significant.

Results

Response to Mechanical Allodynia

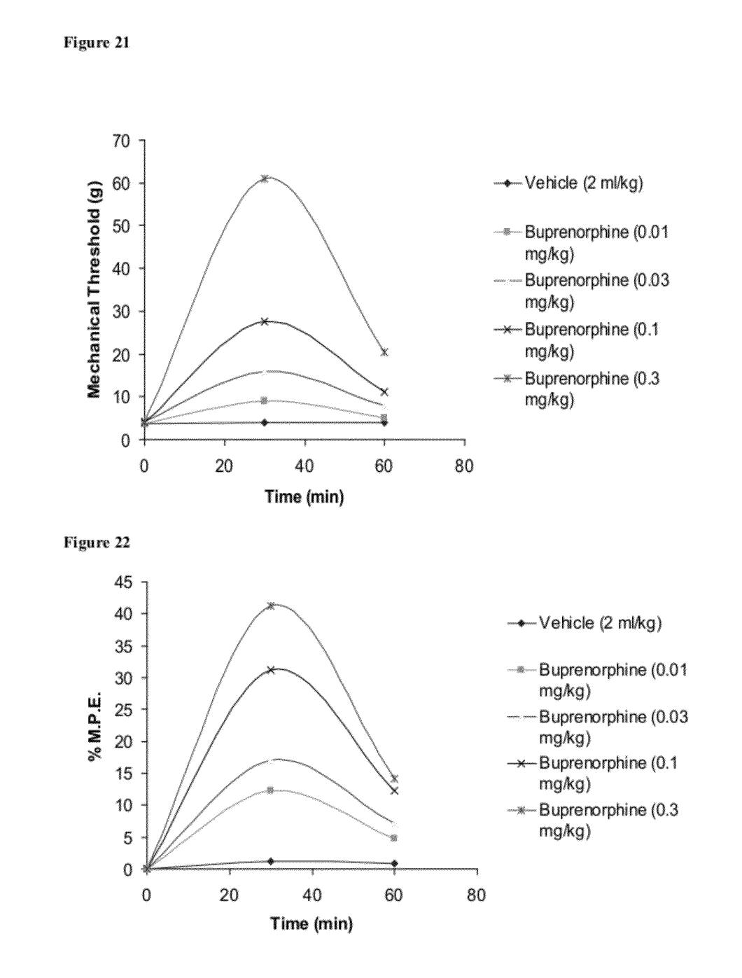

Baseline mean mechanical withdrawal threshold in the paclitaxel treatment induced neuropathic rat was observed to be 3.91+0.12 g. All animals included in the study had a mean threshold below five, thus conforming to the requirements of being considered neuropathic. Oral administration of buprenorphine demonstrated a dose dependent increase in the mechanical withdrawal threshold that elicits pain in the neuropathic rats. Moreover, the effect of the buprenorphine was dependent on the dose and the peak effect of drug was noted to be 30 min post-administration (FIG. 21).

Response to Cold Allodynia

Baseline of cold allodynia score was determined using the acetone drop test in the paclitaxel treatment induced neuropathic rats. Moreover, effect of the drug was dependent on the dose and the peak effect of drug was noted to be 30 min post-administration. Furthermore, the oral administration of buprenorphine demonstrated a dose dependent decrease in the cold allodynia score in the neuropathic rats (FIG. 22).

Conclusion: Oral administration of buprenorphine caused a significant increase in the mechanical allodynia withdrawal threshold and cold allodynia withdrawal threshold in rats with neuropathic pain in comparison to the control readings. This effect of buprenorphine was observed to be dependent of the dose. Thus, buprenorphine demonstrated a marked dose dependent effect in rats with paclitaxel induced neuropathy.

### Spinal Nerve Ligation Induced Peripheral Neuropathy

### Example 67

Aim: The aim of this study was to evaluate the effect of oral administration of buprenorphine in rats with painful peripheral secondary to spinal nerve ligation (SNL). The primary measurements were made mechanical and thermal allodynia designed to detect effects of the treatment(s) on neuropathic pain.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design:

Induction of SNL Neuropathy

The SNL model was adapted from Kim and Chung et al (1992). Rats were anesthetized using thiopental sodium (40 mg/kg, i.p.). The anesthetized animal was fixed on its ventral surface with the hind limbs splayed. A 3 cm longitudinal incision was made on the back around 5 mm lateral to the midline, and above the lower lumbar vertebrae and the rostral sacrum, exposing the paraspinal muscles on the left side. Using a blunt tipped scissors, the paraspinal muscles were isolated and removed from the L4 spinous process to the rostral part of the sacrum, exposing the space ventrolateral to the articular processes, dorsal to the L6 transverse process, and medial to the ileum. The L6 transverse process was removed as completely as possible. Using a piece of 6-0 silk suture, the L5 spinal nerve was tightly ligated. The incision was sutured back in layers. The sutured area was cleaned with 70% ethanol and was sprayed with antiseptic dusting powder. The animals were shifted individually to their home cage and were allowed to recover. Tests took place ten days after the last injection and continued over the next three weeks. Five groups were employed in the present study with each group comprising of 8 animals: Group I (Vehicle control group): Vehicle (2 ml/kg, i.p.) administration was followed by the pain assessment; Group II (Oral buprenorphine treatment group-I): Buprenorphine (0.01 mg/kg, p.o.) administration was followed by the pain assessment; Group III (Oral buprenorphine treatment group-II): Buprenorphine (0.03 mg/kg, p.o.) administration was followed by the pain assessment; Group IV (Oral buprenorphine treatment group-III): Buprenorphine (0.1 mg/kg, p.o.) administration was followed by the pain assessment; Group V (Oral buprenorphine treatment group-IV): Buprenorphine (0.3 mg/kg, p.o.) administration was followed by the pain assessment.

Measurement of Effect

Assessment of Mechanical Allodynia

Mechano-allodynia was assessed using von Frey filaments. The filaments are nylon monofilaments of different diameters that exert defined levels of force to cause the hair to bend. Once a rat was calm, a series of von Frey filaments with roughly exponential incremental target force (0.008, 0.02, 0.04, 0.07, 0.16, 0.4, 0.6, 1, 1.4, 2, 4, 6, 8, 10, 15, 26, 60, 100, 180, 300 g) were applied to the bottom of the right paw, behind the front pads and lateral to the medial pads of the rat. The filament was presented perpendicular to the rat paw and applied with enough force so as to cause a slight bend. It was held steadily against the paw for a period of 5 s. A response was considered positive if, within the 5-s period, the rat withdrew from the stimulus or briskly withdrew immediately after the filament was removed. A negative response was the one in which the rat did not withdraw within the given time. Testing was initiated using middle filament (2 g). A negative response required the use of next filament with greater bending force. A positive response required the use of next filament with less bending force. Results were expressed in grams and determined before and 30 and 60 min after the administration of the test drugs.

Assessment of Cold Allodynia

For the assessment of cold allodynia, which was stable for 3 weeks, the rats were placed on a wire mesh covered with a plastic dome and were allowed to habituate until the exploratory behavior diminished. Cold allodynia was measured as the number of foot withdrawal responses after application of stimuli to the plantar surface of the paw. A drop of acetone (10 μl) was gently applied to the heel of the animal with a micro-pipette. A brisk foot withdrawal response (shaking, tapping, or licking) after the spread of acetone over the plantar region of the paw was considered a sign of cold allodynia. Acetone was applied two times (once every 5 min) on the paw, and the number of reactions (shaking, tapping, or licking) was counted during 30 sec. A cut-off number of reactions were fixed at 20 per trial (i.e. 40). The score was expressed as accumulated numbers of reactions over the trials and determined before and 30 and 60 min after administration of the test drugs. Each individual test was expressed as a percentage of maximum possible effect (% MPE):

$$M P E ( \% ) = \frac{( Post treatment f requency - pre treatment frequency )}{( Cut \text{-} off f requency - pre treatment frequency )} \times 100$$

The peak effect was seen at a time point 30 minutes after the drug administration. MPE (%) at 30 min time point was employed as a quantitative measure of the analgesic effect of the test drugs.

Data Analysis: The anti-nociceptive effect was plotted assessed having time (relative to dosing time point) on the x-axis and percent maximum possible effect (% MPE) on the y-axis. The results were expressed as mean±standard error of means (S.E.M.). Statistical analysis for was done using one-way ANOVA, followed by Tukey's multiple range tests as post-hoc analysis. A value of P<0.05 was considered to be statistically significant.

Results

Response to Mechanical Allodynia

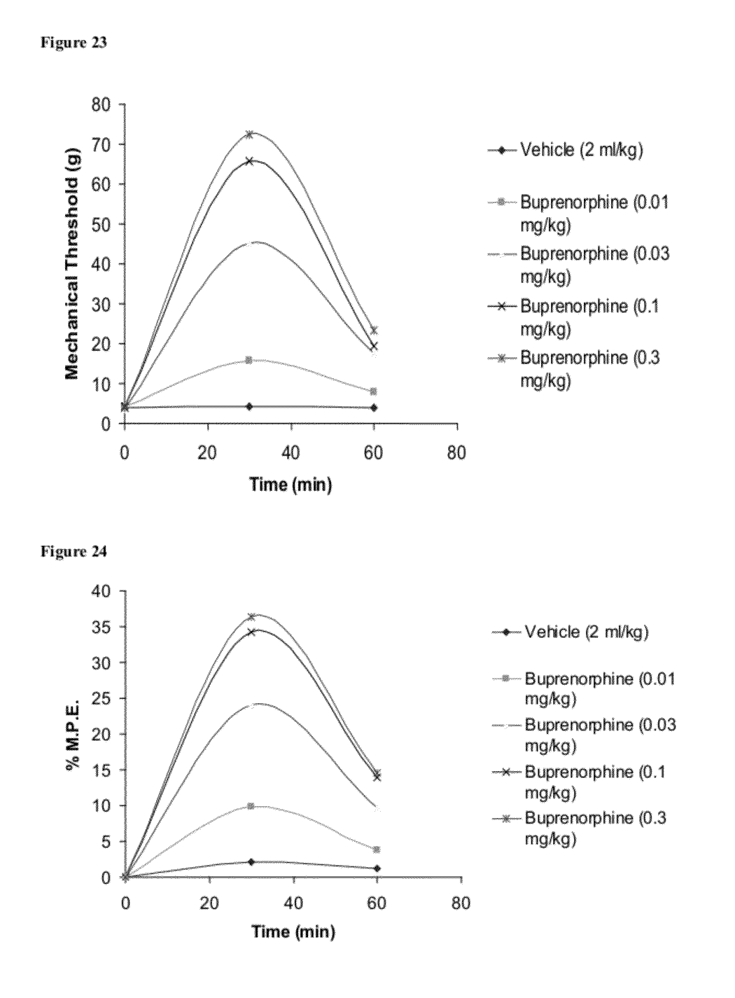

Baseline mean mechanical withdrawal threshold in the SNL induced neuropathy in rats was observed to be 4.01±0.23 g. All animals included in the study had a mean threshold below five, thus conforming to the requirements of being considered neuropathic. Oral administration of buprenorphine demonstrated a dose dependent increase in the mechanical withdrawal threshold that elicits pain in the neuropathic rats. Moreover, the effect of the buprenorphine was dependent on the dose and the peak effect of drug was noted to be 30 min post-administration (FIG. 23).

Response to Cold Allodynia

Baseline of cold allodynia score was determined using the acetone drop test in the SNL induced neuropathic rats. Moreover, effect of the drug was dependent on the dose and the peak effect of drug was noted to be 30 min post-administration. Furthermore, the oral administration of buprenorphine demonstrated a dose dependent decrease in the cold allodynia score in the neuropathic rats (FIG. 24).

Conclusion: Oral administration of buprenorphine caused a significant increase in the mechanical allodynia and cold allodynia withdrawal thresholds in rats with neuropathic pain secondary to spinal nerve ligation, in comparison to the control readings. This effect of buprenorphine was observed to be dependent of the dose. Thus, buprenorphine demonstrated a marked dose dependent effect in rats with vincristine induced neuropathy.

### Streptozotocin Induced Diabetic Neuropathy

### Example 68

Aim: The aim of this study was to evaluate the effect of oral administration of buprenorphine in rats with streptozotocin (STZ) induced diabetes neuropathy. A single injection of streptozotocin (STZ) (40-70 mg/kg, i.p.) was used to induce diabetes in rats. The primary measurements were made mechanical and thermal allodynia designed to detect effects of the treatment(s) on neuropathic pain.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design: The STZ-induced diabetes model was adapted from Arison et al (1967). On the experimental day 1 all rats were fasted 6-8 hrs prior to STZ treatment. Immediately prior to the injection, STZ was dissolved in 50 Mm sodium citrate buffer (pH 4.5) to a final concentration of 10 mg/ml. Using an insulin syringe and a 26 G needle, STZ solution was injected intraperitoneally at a dose level of 40-70 mg/kg. The animals were then shifted individually to their home cage and were provided normal food and 10% sucrose water. On experimental day 2 the sucrose water was switched to regular water. On experimental day 21 all rats were fasted for 6-8 hrs the blood glucose level was tested from a tail vein sample using a spectrophotometeric method in order to check hyperglycemia. Blood glucose levels ranging from 250-600 mg/dL confirmed diabetes in the STZ injected rats. Neuropathy assessment tests were performed after the completion of 8 weeks after STZ treatment. Tests continued over the next four weeks. Five groups were employed in the present study with each group comprising of 8 animals: Group I (Vehicle control group): Vehicle (2 ml/kg, i.p.) administration was followed by the pain assessment; Group II (Oral buprenorphine treatment group-I): Buprenorphine (0.01 mg/kg, p.o.) administration was followed by the pain assessment; Group III (Oral buprenorphine treatment group-II): Buprenorphine (0.03 mg/kg, p.o.) administration was followed by the pain assessment; Group IV (Oral buprenorphine treatment group-III): Buprenorphine (0.1 mg/kg, p.o.) administration was followed by the pain assessment; Group V (Oral buprenorphine treatment group-IV): Buprenorphine (0.3 mg/kg, p.o.) administration was followed by the pain assessment.

Measurement of Effect

Assessment of Mechanical Allodynia

Mechano-allodynia was assessed using von Frey filaments. The filaments are nylon monofilaments of different diameters that exert defined levels of force to cause the hair to bend. Once a rat was calm, a series of von Frey filaments with roughly exponential incremental target force (0.008, 0.02, 0.04, 0.07, 0.16, 0.4, 0.6, 1, 1.4, 2, 4, 6, 8, 10, 15, 26, 60, 100, 180, 300 g) were applied to the bottom of the right paw, behind the front pads and lateral to the medial pads of the rat. The filament was presented perpendicular to the rat paw and applied with enough force so as to cause a slight bend. It was held steadily against the paw for a period of 5 s. A response was considered positive if, within the 5-s period, the rat withdrew from the stimulus or briskly withdrew immediately after the filament was removed. A negative response was the one in which the rat did not withdraw within the given time. Testing was initiated using middle filament (2 g). A negative response required the use of next filament with greater bending force. A positive response required the use of next filament with less bending force. Both hind paws were tested for allodynia and the average of the two was recorded. Results were expressed in grams and determined before and 30 and 60 min after the administration of the test drugs.

Assessment of Cold Allodynia

For the assessment of cold allodynia, which was stable for 3 weeks, the rats were placed on a wire mesh covered with a plastic dome and were allowed to habituate until the exploratory behavior diminished. Cold allodynia was measured as the number of foot withdrawal responses after application of stimuli to the plantar surface of the paw. A drop of acetone (10 μl) was gently applied to the heel of the animal with a micro-pipette. A brisk foot withdrawal response (shaking, tapping, or licking) after the spread of acetone over the plantar region of the paw was considered a sign of cold allodynia. Acetone was applied two times (once every 5 min) on the left paw, and the number of reactions (shaking, tapping, or licking) was counted during 30 sec. A cut-off number of reactions were fixed at 20 per trial (i.e. 40). The score was expressed as accumulated numbers of reactions over the trials and determined before and 30 and 60 min after administration of the test drugs. Each individual test was expressed as a percentage of maximum possible effect (% MPE):

$$M P E ( \% ) = \frac{( Post treatment f requency - pre treatment frequency )}{( Cut \text{-} off f requency - pre treatment frequency )} \times 100$$

The peak effect was seen at a time point 30 minutes after the drug administration. MPE (%) at 30 min time point was employed as a quantitative measure of the analgesic effect of the test drugs.

Data Analysis: The anti-nociceptive effect was plotted assessed having time (relative to dosing time point) on the x-axis and percent maximum possible effect (% MPE) on the y-axis. The results were expressed as mean±standard error of means (S.E.M.). Statistical analysis for was done using one-way ANOVA, followed by Tukey's multiple range tests as post-hoc analysis. A value of P<0.05 was considered to be statistically significant.

Results

Response to Mechanical Allodynia

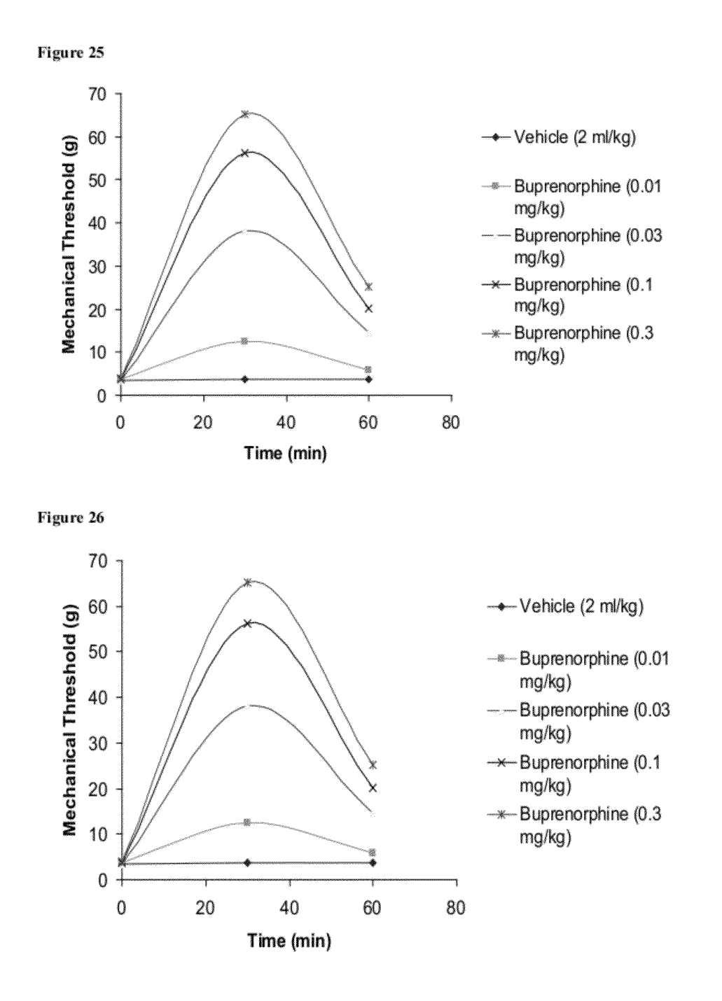

Baseline mean mechanical withdrawal threshold in the diabetes induced neuropathy in rats was observed to be 3.65±0.31 g. All animals included in the study had a mean threshold below five, thus conforming to the requirements of being considered neuropathic. Oral administration of buprenorphine demonstrated a dose dependent increase in the mechanical withdrawal threshold that elicits pain in the neuropathic rats. Moreover, the effect of the buprenorphine was dependent on the dose and the peak effect of drug was noted to be 30 min post-administration (FIG. 25).

Response to Cold Allodynia

Baseline of cold allodynia score was determined using the acetone drop test in the SNL induced neuropathic rats. Moreover, effect of the drug was dependent on the dose and the peak effect of drug was noted to be 30 min post-administration. Furthermore, the oral administration of buprenorphine demonstrated a dose dependent decrease in the cold allodynia score in the neuropathic rats (FIG. 26).

Conclusion: Oral administration of buprenorphine caused a significant increase in the mechanical allodynia and cold allodynia withdrawal thresholds in rats with STZ induced diabetic neuropathic pain in comparison to the control readings. This effect of buprenorphine was observed to be dependent of the dose. Thus, buprenorphine demonstrated a marked dose dependent effect in rats with STZ induced diabetic neuropathic pain.

### Example 69

Aim: The aim of this study was to evaluate the analgesic effect of orally administered buprenorphine following administration into different parts of the gastrointestinal tract in transiently ether anaesthetized rats, using the formalin test of persistent pain. The primary measurements were made using the paw flinching frequency in the formalin test designed to detect effects of the treatment(s) on nociception in rats.

Animals: Wistar rats weighing 250±20 g were maintained on standard laboratory diet and having free access to tap water ad lib were employed in the present study. They were housed in the animal house and were exposed to 12 hr cycle of light and dark. The experiments were conducted in a semi-sound proof laboratory. Care of the animals was in accordance with guidelines for experimentation on animals.

Experimental Design: Four groups were employed in the present study with each group comprising of 8 animals. Each animal was subjected to transient ether anesthesia followed by a minor incision in the abdominal cavity through which buprenorphine 0.2 mg/kg was administered using a syringe into the stomach, ileum and colon. The control group underwent sham surgery. Flinching frequency was observed every 5 minutes for 15 seconds.

Measurement of Effect: Rats were placed in open Plexiglas boxes to acclimate to the environment for 30 min and then were injected subcutaneously with 50 μl of 5% formalin solution at the plantar surface of the right paw, using a 27-gauge needle. After the injection, rats displayed a characteristic nociceptive behavior in the form a flinching response. Two phases of nociceptive behaviors were observed. Phase 1 was initiated within seconds after injection and lasted for about 5-10 min. After several minutes quiescent, a second phase of flinching occurred and peaked at 30-40 min after the formalin injection. The flinching frequency was observed every 5 min each time for an observation period of 15 sec. The time response data are presented as the number of flinches versus time.

Data Analysis: The anti-nociceptive effect following intragastric, intra-ileal and intra-colonic administration was assessed in terms of the number of flinches versus time. The results were expressed as mean±standard error of means (S.E.M.). Statistical analysis for was done using one-way ANOVA, followed by Tukey's multiple range tests as post-hoc analysis. A value of P<0.05 was considered to be statistically significant.

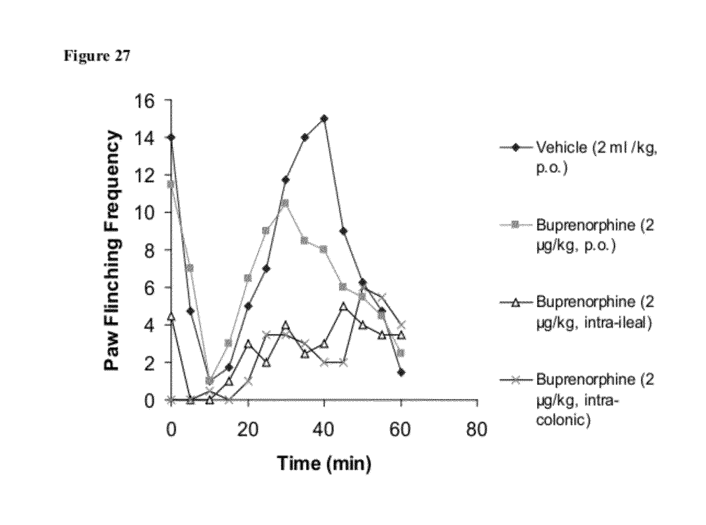

Results: Two phases of flinching behaviours were observed after paw formalin injections in the rats. Phase 1 was initiated immediately after formalin injection and it subsided until about 10 min after injection. Thereafter, a second phase flinching response appeared, peaked at around 30-40 min after injection. Intra-gastric, intra-ileal, and intra-colonic administration of buprenorphine each caused a decrease in flinching responses in both phase 1 and phase 2 in rats, compared with untreated controls. Buprenorphine induced differential reduction in the flinching response in the formalin test. However, intra-gastric administration of buprenorphine produced only a modest analgesic effect, while both intra-ileal administration and intra-colonic administration of buprenorphine produced a robust analgesic response. The analgesic effects after intra-ileal and intra-colonic administration were significantly greater than those following intra-gastric administration of buprenorphine (see FIG. 27).

### Side Effects with Sublingual/Oral Buprenorphine

### Example 70

Twenty opioid naïve subjects with chronic musculoskeletal pain were treated with a single dose of sublingual buprenorphine 0.4 mg (n=10; 8 M; 2 F; age range: 28 to 67 year) or oral buprenorphine 0.4 mg (n=10; 8 m:2 F; age range 22 to 61 years). Drowsiness and nausea were assessed prior to the start of treatment and at 0.5, 1, 2, 3, 4, 5 and 6 hours using a 0 to 5 scale. The mean drowsiness and nausea scores were greater after sublingual buprenorphine than after oral buprenorphine. Oral buprenorphine was much better tolerated than sublingual buprenorphine.

### Pain Relief with Modified Release Buprenorphine

### Example 71

The therapeutic effect of repeated dose oral buprenorphine was evaluated in patients with chronic pain by comparing oral immediate release buprenorphine with oral modified release buprenorphine intended for delayed onset, rapid release buprenorphine. Oral immediate release buprenorphine was prepared by encapsulating the required number of sublingual buprenorphine 0.2 mg tablets. Oral delayed onset, rapid release buprenorphine was similarly prepared. However, following encapsulation, the delayed onset, rapid release dosage form was coated using the coating method generally described in Example 56 to provide release at a pH>7. Ten subjects with chronic low back pain (ages 48 to 61 years) and 10 patients with knee and/or hip osteoarthritis (ages 46 to 64 years), all with moderate to severe pain received oral immediate release buprenorphine or delayed onset, rapid release buprenorphine for one week (5 to 7 days), followed by crossover to the alternate treatment. The buprenorphine dose was fixed at 0.5 mg TID (about every 8 hours). Patients were asked to evaluate their average pain intensity score once a day in the evening and report to the clinic at the end of each treatment week. Both oral immediate release buprenorphine and delayed onset, rapid release buprenorphine provided pain relief. However, the pain relief in the overall treatment group receiving delayed onset, rapid release was greater than after oral immediate release buprenorphine on all days except Day 1. Oral immediate release buprenorphine provided greater pain relief than delayed onset, rapid release on Day 1 of treatment.

Pharmacokinetics of Oral Buprenorphine

The rate and extent of absorption (bioavailability) of oral buprenorphine is evaluated as shown in the prophetic pharmacokinetic study example below comparing sublingual buprenorphine and oral buprenorphine. The pharmacokinetics of a drug can vary considerably from study to study due to both study and patient related variables, most notably, the intra- and inter-patient variability is drug liberation, absorption, distribution, metabolism, and excretion (LADME). This is an issue of particular concern for buprenorphine pharmacokinetics, particularly when given by the sublingual route of administration. Consequently, the results below should be considered illustrative but not limiting of the expected pharmacokinetics of oral, extended release buprenorphine and oral immediate release buprenorphine.

### Example 72

Oral Extended Release Buprenorphine

| Design | Single-dose, fasted, randomized cross-over (2 week | |
|---|---|---|
| | washout) evaluation of oral extended release | |
| | buprenorphine and sublingual buprenorphine under | |
| | naltrexone protection. | |
| Subjects | 8 healthy volunteers | |
| Study Test Drugs | Oral Buprenorphine HCl Extended Release 2 × 10 mg | |
| | (20 mg of buprenorphine HCl) or Sublingual | |
| | Buprenorphine HCl 1 × 8 mg (8 mg of buprenorphine | |
| | base) | |
| Other Study Drugs | Naltrexone HCl 25 mg 12 hours prior to, 2 hours prior | |
| | to and 10 hours after dosing with study test drugs. | |
| Pharmacokinetic | Blood sampling for buprenorphine at pre-dose and at | |
| Sampling | 0.25, 0.5, 0.75, 1, 1.5, 2, 2.5, 3, 4, 5, 6, 7, 8, 10, 12, | |
| | 14, 16, 20, 24, 28, 36, and 48 hours post-dose. | |
| Analytical Method | LCMS/MS | |
| Data Analysis | Plasma concentrations dose normalized to 8 mg | |
| | buprenorphine base. | |
| Pharmacokinetic | Buprenorphine HCl | |
| Parameter | (Extended Release | Buprenorphine HCl |
| (Buprenorphine) | 2 × 10 mg) | (Sublingual 1 × 8 mg) |
| Cmax (pg/mL) | 653 | 2819 |
| Tmax (hours) | 3.71 | 1.23 |
| AUC₀₋₄₈ (pg · hr/mL) | 10129 | 19736 |
| AUC_{0-inf} (pg · hr/mL) | 18472 | 28429 |
| Pharmacokinetic | | |
| Parameter | Buprenorphine HCl | Sublingual |
| (Norbuprenorphine) | Extended Release | Buprenorphine HCl |
| Cmax (pg/mL) | 598 | 801 |
| Tmax (hours) | 4.55 | 5.17 |
| AUC₀₋₄₈ (pg · hr/mL) | 18235 | 16483 |
| AUC_{0-inf} (pg · hr/mL) | 39394 | 69411 |

### Example 73

Delayed Onset, Rapid Release Oral Buprenorphine

| Design | Single-dose, fasted, randomized cross-over (2 week | |
|---|---|---|
| | washout) evaluation of delayed onset, rapid release | |
| | oral buprenorphine and sublingual buprenorphine | |
| | under naltrexone protection. | |
| Subjects | 12 healthy volunteers | |
| Study Test Drugs | Delayed onset, rapid release oral buprenorphine HCl | |
| | 1 × 8 (8 mg of buprenorphine HCl) or sublingual | |
| | buprenorphine HCl 1 × 8 mg (8 mg of buprenorphine | |
| | base) | |
| Other Study Drugs | Naltrexone HCl 25 mg 12 hours prior to, 2 hours prior | |
| | to and 10 hours after dosing with study test drugs. | |
| Pharmacokinetic | Blood sampling for buprenorphine at pre-dose and at | |
| Sampling | 0.25, 0.5, 0.75, 1, 1.5, 2, 2.5, 3, 3.5, 4, 4.5, 5, 5.5, 6, | |
| | 6.5, 7, 8, 10, 12, 14, 16, 20, 24, 28, 36, and 48 hours | |
| | post-dose. | |
| Analytical Method | LCMS/MS | |
| Data Analysis | Plasma concentrations dose normalized to 8 mg | |
| | buprenorphine base. | |
| Pharmacokinetic | Buprenorphine HCl | |
| Parameter | (Delayed Onset, Rapid | Buprenorphine HCl |
| (Buprenorphine) | Release Oral 1 × 8 mg) | (Sublingual 1 × 8 mg) |
| Cmax (pg/mL) | 1573 | 2415 |
| Tmax (hours) | 4.41 | 1.21 |
| AUC₀₋₄₈ (pg · hr/mL) | 12622 | 18839 |
| AUC_{0-inf} (pg · hr/mL) | 20142 | 28429 |

### Example 74

Delayed Onset, Extended Release Oral Buprenorphine

| Design | Single-dose, fasted, randomized cross-over (2 week | |
|---|---|---|
| | washout) evaluation of delayed onset, extended | |
| | release oral buprenorphine and sublingual | |
| | buprenorphine under naltrexone protection. | |
| Subjects | 12 healthy volunteers | |
| Study Test Drugs | Delayed onset, extended release oral buprenorphine | |
| | HCl 2 × 10 (8 mg of buprenorphine HCl) or | |
| | sublingual buprenorphine HCl 1 × 8 mg (8 mg of | |
| | buprenorphine base) | |
| Other Study Drugs | Naltrexone HCl 25 mg 12 hours prior to, 2 hours prior | |
| | to and 10 hours after dosing with study test drugs. | |
| Pharmacokinetic | Blood sampling for buprenorphine at pre-dose and at | |
| Sampling | 0.25, 0.5, 0.75, 1, 1.5, 2, 2.5, 3, 3.5, 4, 4.5, 5, 5.5, 6, | |
| | 6.5, 7, 8, 10, 12, 14, 16, 20, 24, 28, 36, and 48 hours | |
| | post-dose. | |
| Analytical Method | LCMS/MS | |
| Data Analysis | Plasma concentrations dose normalized to 8 mg | |
| | buprenorphine base. | |
| | Buprenorphine HCl | |
| Pharmacokinetic | (Delayed Onset, | |
| Parameter | Extended Release Oral | Buprenorphine HCl |
| (Buprenorphine) | 2 × 10 mg) | (Sublingual 1 × 8 mg) |
| Cmax (pg/mL) | 707 | 2710 |
| Tmax (hours) | 7.12 | 1.31 |
| AUC₀₋₄₈ (pg · hr/mL) | 13601 | 20850 |
| AUC_{0-inf} (pg · hr/mL) | 20694 | 29328 |

These and other embodiments of the present invention will readily occur to those of ordinary skill in the art in view of the disclosure herein.

The included examples are illustrative but not limiting of the methods and composition of the present invention. Other suitable modifications and adaptations of the variety of conditions and parameters normally encountered and obvious to those skilled in the art are within the spirit and scope of the invention.

A wide variety of materials can be used for preparing the dosage form according to this invention. This invention therefore contemplates the use of materials other than those specifically disclosed herein, including those which may hereafter become known to the art to be capable of performing the necessary functions.

In all instances wherein prophetic examples are provided, these compositions are intended to be exemplary and it should be understood that the specific procedures, constituents, amounts thereof and the like can be varied in order to obtain a composition possessing desired properties.

Having now fully described the invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

1. An oral dosage form comprising: (i) a therapeutically effective amount of buprenorphine, a pharmaceutically acceptable salt thereof, or a mixture thereof, and (ii) controlled release material to render said dosage form suitable for modified release, said dosage form providing delayed onset of buprenorphine release.

2-5. (canceled)

6. The oral dosage form of claim 1, being abuse resistant compared with oral dosage forms of buprenorphine suitable for modified release or extended release or controlled release but without delayed release.

7. The dosage form of claim 6, wherein the dose of said modified release dosage form with delayed onset is at least about 10% greater than the comparator dose.

8-18. (canceled)

19. The dosage form of claim 1, wherein the modified release dosage form is a delayed onset, rapid release dosage form.

20. The dosage form of claim 1, wherein the modified release dosage form is a delayed onset, pulsatile release dosage form.

21. The dosage form of claim 1, wherein the modified release dosage form is a delayed onset, extended release dosage form.

22-54. (canceled)

55. The oral dosage form of claim 1, providing first release of buprenorphine from the dosage form at a point distal to the stomach.

56. The oral dosage form of claim 55, wherein said first release is at a point distal to the duodenum.

57. The oral dosage form of claim 55, wherein said first release is at a point distal to the Jejunum.

58. The oral dosage form of claim 55, wherein said first release is at a point distal to the ileum.

59. The oral dosage form of claim 55, wherein said first release is at a point distal to the ileocecal junction.

60. The oral dosage form of claim 1, after a single administration providing first release of buprenorphine from the dosage form not less than about 2 hours after oral ingestion.

61-69. (canceled)

70. The oral dosage form of claim 1, after a single administration providing first release of buprenorphine from the dosage form at a pH of about ≧5.

71-79. (canceled)

80. The oral dosage form of claim 1, upon initiating release providing delayed onset, rapid release of buprenorphine for up to about 3 hours.

81. The oral dosage form of claim 1, said upon initiating release providing delayed onset, extended release of buprenorphine for up to about 24 hours.

82-111. (canceled)

112. The oral dosage form of claim 1, providing an in-vitro buprenorphine release rate, when measured by the USP Basket or Paddle Method at 100 rpm in 900 mL of distilled water at 37° C. which is substantially pH dependent in that a difference, at 2 hours between the amount of buprenorphine released at a pH of ≦5, and an amount released at a pH of ≧6 is more than about 25%.

113-115. (canceled)

116. An oral dosage form comprising a therapeutically effective amount of buprenorphine, a pharmaceutically acceptable salt thereof, or a mixture thereof, and naloxone, a pharmaceutically acceptable salt thereof, or a mixture thereof, said dosage form having a mass ratio of naloxone base relative to buprenorphine base which is at least 50% less than the mass ratio of naloxone base relative to buprenorphine base contained in the commercially marketed sublingual preparation containing buprenorphine and naloxone.

117. The oral dosage form of claim 116, further comprising controlled release material to render said dosage form suitable for modified release.

118-126. (canceled)

127. The oral dosage form of claim 116, wherein the mass ratio of naltrexone base to buprenorphine base is 1:8 or less.

128-210. (canceled)

211. An oral modified release pharmaceutical composition comprising a therapeutically effective amount of buprenorphine, a pharmaceutically acceptable salt thereof or a mixture thereof; and a controlled release material to render said dosage form suitable for extended release, or delayed onset extended release in a mammal.

212-374. (canceled)
